(19) United States
(12) Patent Application Publication
(10) Pub. No.: US 2025/0312376 A1
(43) Pub. Date: Oct. 9, 2025

(54) COMPOSITIONS AND METHODS FOR MODULATING A GENOME IN T CELLS, INDUCED PLURIPOTENT STEM CELLS, AND RESPIRATORY EPITHELIAL CELLS

(71) Applicant: FLAGSHIP PIONEERING INNOVATIONS VI, LLC, Cambridge, MA (US)

(72) Inventors: Barrett Ethan Steinberg, Somerville, MA (US); Cecilia Giovanna Silvia Cotta-Ramusino, Cambridge, MA (US); Jason John Rodriguez, Watertown, MA (US); Alberto De laco, Cambridge, MA (US); Donghui Li, Somerville, MA (US); Kyusik Kim, Worcester, MA (US); Pin-Fang Chen, Boston, MA (US); Anne Helen Bothmer, Cambridge, MA (US); Aamir Mir, Medford, MA (US); Giulia Schiroli, Boston, MA (US)

(21) Appl. No.: 18/860,423

(22) PCT Filed: Apr. 28, 2023
(86) PCT No.: PCT/US2023/066398

§ 371 Date: Oct. 25, 2024

Related U.S. Application Data
(60) Provisional application No. 63/363,806, filed on Apr. 28, 2022.
(60) Provisional application No. 63/366,173, filed on Jun. 10, 2022.
(60) Provisional application No. 63/378,360, filed on Oct. 4, 2022.
(60) Provisional application No. 63/478,930, filed on Jan. 7, 2023.
(60) Provisional application No. 63/491,439, filed on Mar. 21, 2023.

(51) Int. Cl. A61K 35/17; A61K 40/11; A61K 40/31; A61K 40/42; A61P 35/00; C07K 14/705; C07K 14/725; C12N 5/074; C12N 9/12; C12N 15/11; C12N 15/85; C12N 15/90

(52) U.S. Cl. CPC A61K 35/17; A61K 40/11; A61K 40/31; A61K 40/4215; A61P 35/00; C07K 14/7051; C07K 14/70517; C07K 14/70521; C12N 5/0696; C12N 9/1276; C12N 15/111; C12N 15/85; C12N 15/907; A61K 2239/17; A61K 2239/21; A61K 2239/48; C07K 2317/53; C07K 2319/02; C07K 2319/03; C12N 2310/20; C12N 2510/00

(57) ABSTRACT

Methods and compositions for modulating a target genome are disclosed. For instance, gene modifying systems may be used to insert a heterologous object sequence (e.g., encoding a chimeric antigen receptor) into a target cell. The target cell may be, e.g., a T cell, induced pluripotent stem cell, or respiratory epithelial cell.

### CROSS REFERENCE TO RELATED APPLICATIONS

This application claims the benefit of U.S. Provisional Application No. 63/363,806, filed Apr. 28, 2022, U.S. Provisional Application No. 63/366,173, filed Jun. 10, 2022, U.S. Provisional Application No. 63/378,360, filed Oct. 4, 2022, U.S. Provisional Application No. 63/478,930, filed Jan. 7, 2023, and U.S. Provisional Application No. 63/491,439, filed Mar. 21, 2023. The contents of the aforementioned applications are hereby incorporated by reference in their entirety.

### SEQUENCE LISTING

The instant application contains a Sequence Listing which has been submitted electronically in XML format compliant with WIPO Standard ST.26 and is hereby incorporated by reference in its entirety. Said XML copy, created on Apr. 27, 2023, is named V2065-7031WO_SL.xml and is 3,979,000 bytes in size.

### BACKGROUND

Integration of a nucleic acid of interest into a genome occurs at low frequency and with little site specificity, in the absence of a specialized protein to promote the insertion event. Some existing approaches, like CRISPR/Cas9, are more suited for small edits and are less effective at integrating longer sequences. Other existing approaches, like Cre/loxP, require a first step of inserting a loxP site into the genome and then a second step of inserting a sequence of interest into the loxP site. There is a need in the art for improved proteins for inserting sequences of interest into a genome.

### SUMMARY OF THE INVENTION

This disclosure relates to novel compositions, systems and methods for altering a genome at one or more locations in a host cell, tissue or subject, in vivo, in vitro, or ex vivo. In particular, the invention features compositions, systems and methods for the introduction of exogenous genetic elements into a host genome. The disclosure also provides systems for altering a genomic DNA sequence of interest, e.g., by inserting, deleting, or substituting one or more nucleotides into/from the sequence of interest.

Features of the compositions or methods can include one or more of the following enumerated embodiments.
1. A system for modifying DNA comprising:
   (a) a gene modifying polypeptide or a nucleic acid (e.g., DNA or mRNA) encoding the gene modifying polypeptide, and
   (b) a template RNA (or DNA encoding the template RNA) comprising (i) a sequence that binds the polypeptide and (ii) a heterologous object sequence encoding a chimeric antigen receptor (CAR), wherein the CAR comprises an antigen-binding domain, a transmembrane domain, a first intracellular signaling domain, and a second intracellular signaling domain.
2. A system for modifying DNA comprising:
   (a) a gene modifying polypeptide or a nucleic acid (e.g., DNA or mRNA) encoding the gene modifying polypeptide, and
   (b) a template RNA (or DNA encoding the template RNA) comprising (i) a sequence that binds the polypeptide and (ii) a heterologous object sequence encoding a chimeric antigen receptor (CAR), wherein one or more of:
   (i) the CAR comprises an antigen binding domain that binds one or more antigens of a blood cancer (e.g., a leukemia or lymphoma), wherein optionally the antigen is a B cell antigen; (ii) the CAR comprises an antigen binding domain that binds one or more antigens of a solid tumor;
   (iii) the CAR comprises an antigen binding domain of any one of Tables C1-C5 or C9; (iv) the CAR comprises a linker domain of Table L1 (e.g., a linker of SEQ ID NO 15520);
   (v) the CAR comprises a transmembrane domain of Table C6 or C6A;
   (vi) the CAR comprises a hinge domain (e.g., a hinge domain of Table C8);
   (vii) the CAR comprises an intracellular signaling domain of Table C7 or C7A;
   (viii) the CAR comprises a costimulatory domain of Table C7 or C7A;
   (ix) the CAR comprises an antigen binding domain which comprises an scFv, a Fab, a diabody, a D domain binder, a centryin, or one or more single domain antibodies (e.g., VHH domains); or
   (x) the CAR comprises an amino acid sequence of Table C9 or an amino acid sequence according to any one of SEQ ID NOs: 1100, 15490, 15492, 15498, 15500, 15502, 15503, 15505, 15507, 15509 and 15510, 15555, 15557 and 15558, 15559, 15560, 15561, 15515, 15526, 15531, 15536, 15541, or 15548;
   (xi) wherein the CAR comprises a first intracellular signaling domain and a second intracellular signaling domain.
3. The system of embodiment 1 or 2, wherein the first intracellular signaling domain mediates downstream signaling during T-cell activation.
4. The system of any of embodiments 1-3, wherein the second intracellular signaling domain is a costimulatory domain.
5. A population of cells comprising immune effector cells and/or regulatory immune cells, the population comprising:
   a plurality of copies of a gene encoding a CAR (“a CAR gene”), wherein less than or equal to 70%, 65%, 60%, or 55% of copies of the CAR gene in the population are situated within a gene endogenous to a cell of the population.
6. A population of cells comprising immune effector cells and/or regulatory immune cells, the population comprising:
   a plurality of copies of a gene encoding a CAR (“a CAR gene”), wherein less than 10%, 9%, 8%, 7%, 6%, or 5% of copies of the CAR gene in the population are situated within an exon of a gene endogenous to a cell of the population.
7. A population of cells comprising immune effector cells and/or regulatory immune cells, the population comprising:
   a plurality of copies of a gene encoding a CAR (“a CAR gene”), wherein less than 70%, 65%, 60%, 55%, or 50% of copies of the CAR gene in the population are situated within an intron of a gene endogenous to a cell of the population.
8. A population of cells comprising immune effector cells and/or regulatory immune cells, the population comprising:
   a plurality of copies of a gene encoding a CAR (“a CAR gene”), wherein less than 10%, 9%, 8%, or 7% of copies of the CAR gene in the population are situated upstream of a gene (e.g., within 2 kb of a transcriptional start site (TSS)) endogenous to a cell of the population.
9. A population of cells comprising immune effector cells and/or regulatory immune cells, the population comprising:
   a plurality of copies of a gene encoding a CAR (“a CAR gene”), wherein
   at least 20%, 25%, 30%, 35%, or 40% of copies of the CAR gene in the population are situated within an intergenic region endogenous to a cell of the population.
10. The population of cells of any of embodiments 5-9, wherein one or more of:
   less than or equal to 70%, 65%, 60%, or 55% of copies of the CAR gene in the population are situated within a gene endogenous to a cell of the population;
   less than 10%, 9%, 8%, 7%, 6%, or 5% of copies of the CAR gene in the population are situated within an exon of a gene endogenous to a cell of the population;
   less than 70%, 65%, 60%, 55%, or 50% of copies of the CAR gene in the population are situated within an intron of a gene endogenous to a cell of the population;
   less than 10%, 9%, 8%, or 7% of copies of the CAR gene in the population are situated upstream of a gene (e.g., within 2 kb of a transcriptional start site (TSS)) endogenous to a cell of the population; or
   at least 20%, 25%, 30%, 35%, or 40% of copies of the CAR gene in the population are situated within an intergenic region endogenous to a cell of the population.
11. The population of cells of any of embodiments 5-10, wherein at least 50%, 55%, 60%, 65%, 70%, 75%, 80%, 85%, 90%, 95%, 96%, 97%, 98%, 99%, or 100% of cells in the plurality comprises a single copy of the CAR gene.
12. The population of cells of any of embodiments 5-10, wherein each cell in the plurality comprises a single copy of the CAR gene.
13. The population of cells of any of embodiments 5-10, wherein at least 0.1%, 1%, 5%, 10%, 15%, 20%, 25%, 30%, or 35%, or 1%-5%, 5%-10%, 10%-15%, 15%-20%, 20%-25%, 25%-30%, or 30%-35% of cells in the population comprise the CAR gene.
14. The population of cells of any of embodiments 5-13, which comprises one or more of cancer cells, T effector cells, T helper cells, regulatory T cells, monocytes, and NK cells.
15. The population of cells of any of embodiments 5-14, wherein the immune effector cells and/or regulatory immune cells comprise T cells, e.g., primary T cells.
16. The population of cells of any of embodiments 5-15, wherein the immune effector cells and/or regulatory immune cells comprise a leukapheresis sample or an apheresis sample.
17. The population of cells of any of embodiments 5-16, which is substantially free of lentivirus proteins.
18. The population of cells of any of embodiments 5-17, which is substantially free of lentivirus nucleic acids.
19. A method of modifying the genome of a mammalian cell, comprising contacting the cell with a system of any of embodiments 1-4, thereby modifying the genome of the mammalian cell.
20. The method of embodiment 19, wherein the mammalian cell is a T cell, e.g., a primary T cell.
21. A reaction mixture comprising:
   a system of any of embodiments 1-4, and
   a mammalian cell.
22. A method of modifying the genome of a mammalian T cell (e.g., a primary T cell), the method comprising contacting the cell with:
   (a) a gene modifying polypeptide comprising an amino acid sequence of Table R1 or E14 or a sequence at least 70%, 75%, 80%, 85%, 90%, 95%, 96%, 97%, 98%, or 99% identical thereto, or a nucleic acid (e.g., DNA or mRNA) encoding the gene modifying polypeptide, and
   (b) a template RNA (or DNA encoding the template RNA) comprising (i) a sequence that binds the polypeptide and (ii) a heterologous object sequence.
23. A method of modifying the genome of a mammalian T cell (e.g., a primary T cell), the method comprising contacting the cell with:
   (a) a gene modifying polypeptide comprising an amino acid sequence of Table R1 or E14 or a sequence having no more than 10, 20, 30, 40, 50, 60, 70, 80, 90, or 100 amino acid differences thereto, or a nucleic acid (e.g., DNA or mRNA) encoding the gene modifying polypeptide, and
   (b) a template RNA (or DNA encoding the template RNA) comprising (i) a sequence that binds the polypeptide and (ii) a heterologous object sequence.
24. The method of any of embodiments 19, 20, 22, or 23, which is performed ex vivo or in vitro.
25. The method of any of embodiments 19, 20, or 22-24, wherein the formulated with an LNP.
26. The method of any of embodiments 19, 20, or 22-25, which results in insertion of the heterologous object sequence into at least 1%, 5%, 10%, 15%, 20%, 25%, 30%, or 35% or 1%-5%, 5%-10%, 10%-15%, 15%-20%, 20%-25%, 25%-30%, or 30%-35% of T cells.
27. A reaction mixture comprising:
   a gene modifying polypeptide comprising an amino acid sequence of Table R1 or E14 or a sequence at least 70%, 75%, 80%, 85%, 90%, 95%, 96%, 97%, 98%, or 99% identical thereto, or a nucleic acid (e.g., DNA or mRNA) encoding the gene modifying polypeptide,
   optionally, a template RNA (or DNA encoding the template RNA) comprising (i) a sequence that binds the polypeptide and (ii) a heterologous object sequence;
   a mammalian T cell (e.g., a primary T cell).
28. A reaction mixture comprising:
   a gene modifying polypeptide comprising an amino acid sequence of Table R1 or E14 or a sequence no more than 10, 20, 30, 40, 50, 60, 70, 80, 90, or 100 amino acid differences thereto, or a nucleic acid (e.g., DNA or mRNA) encoding the gene modifying polypeptide,
   optionally, a template RNA (or DNA encoding the template RNA) comprising (i) a sequence that binds the polypeptide and (ii) a heterologous object sequence;
   a mammalian T cell (e.g., a primary T cell).
29. A cell or population of cells produced by the method of any of embodiments 19, 20, 22-26.
30. The population of cells of embodiment 29, wherein less than or equal to 70%, 65%, 60%, or 55% of copies of the heterologous object sequence in the population are situated within a gene endogenous to a cell of the population.
31. The population of cells of embodiment 29 or 30, wherein less than 10%, 9%, 8%, 7%, 6%, or 5% of copies of the heterologous object sequence in the population are situated within an exon of a gene endogenous to a cell of the population.
32. The population of cells of any of embodiments 29-31, wherein less than 70%, 65%, 60%, 55%, or 50% of copies of the heterologous object sequence in the population are situated within an intron of a gene endogenous to a cell of the population.
33. The population of cells of any of embodiments 29-32, wherein less than 10%, 9%, 8%, or 7% of copies of the heterologous object sequence in the population are situated upstream of a gene (e.g., within 2 kb of a transcriptional start site (TSS)) endogenous to a cell of the population.
34. The population of cells of any of embodiments 29-33, wherein at least 20%, 25%, 30%, 35%, or 40% of copies of the heterologous object sequence in the population are situated within an intergenic region endogenous to a cell of the population.
35. A method of treating a cancer in a subject in need thereof, the method comprising administering to the subject a cell or population of cells of any of embodiments 5-18 or 29-34.
36. A cell or population of cells of any of embodiments 5-18 or 29-34 or the system of any of embodiments 1-4, for use in treating a cancer.
37. Use of a cell or population of cells of any of embodiments 5-18 or 29-34 or the system of any of embodiments 1-4, in the manufacture of a medicament for treating a cancer.
38. A method of treating a cancer in a subject in need thereof, the method comprising contacting an immune effector cell and/or regulatory immune cell of the subject with a system of any of embodiments 1-4.
39. A gene modifying polypeptide comprising an amino acid sequence of SEQ ID NO: 420, or a sequence having at least 70%, 75%, 80%, 85%, 90%, 95%, 96%, 97%, 98%, or 99% identity thereto, wherein amino acid position 191 is other than D, e.g., is A, or a fragment thereof having reverse transcriptase activity.
40. A gene modifying polypeptide comprising an amino acid sequence of SEQ ID NO: 420, or a sequence having no more than 10, 20, 30, 40, 50, 60, 70, 80, 90, or 100 nucleotide differences thereto, wherein amino acid position 191 is other than D, e.g., is A, or a fragment thereof having reverse transcriptase activity.
41. The gene modifying polypeptide of embodiment 39 or 40, which has an endonuclease activity of less than 20%, 15%, 10%, or 5% of that of a polypeptide of SEQ ID NO: 405 in an assay according to Example 4.
42. A gene modifying polypeptide comprising an amino acid sequence of SEQ ID NO: 421, or a sequence having at least 70%, 75%, 80%, 85%, 90%, 95%, 96%, 97%, 98%, or 99% identity thereto, wherein amino acid position 250 is other than D, e.g., is A, or a fragment thereof having reverse transcriptase activity.
43. A gene modifying polypeptide comprising an amino acid sequence of SEQ ID NO: 421, or a sequence having no more than 10, 20, 30, 40, 50, 60, 70, 80, 90, or 100 nucleotide differences thereto, wherein amino acid position 191 is other than D, e.g., is A, or a fragment thereof having reverse transcriptase activity.
44. The gene modifying polypeptide of embodiment 42 or 43, which has an endonuclease activity of less than 20%, 15%, 10%, or 5% of that of a polypeptide of SEQ ID NO: 406 in an assay according to Example 4.
45. The gene modifying polypeptide of any of embodiments 39-44, which further comprises a heterologous protein domain.
46. The gene modifying polypeptide of embodiment 45, wherein a linker is disposed between the heterologous protein domain and the amino acid sequence having at least 70%, 75%, 80%, 85%, 90%, 95%, 96%, 97%, 98%, or 99% identity to a linker in Table L1, or fragment thereof.
47. A nucleic acid encoding a gene modifying polypeptide of any of embodiments 39-46.
48. A method of modifying the genome of a mammalian induced pluripotent stem cell (iPSC), the method comprising contacting the cell with:
   (a) a gene modifying polypeptide, or a nucleic acid (e.g., DNA or mRNA) encoding the gene modifying polypeptide, and
   (b) a template RNA (or DNA encoding the template RNA) comprising (i) a sequence that binds the polypeptide and (ii) a heterologous object sequence.
49. The method of embodiment 48, which results in insertion of the heterologous object sequence into at least 5%, 10%, or 15% of iPSCs.
50. A reaction mixture comprising:
   a gene modifying polypeptide, or a nucleic acid (e.g., DNA or mRNA) encoding the gene modifying polypeptide,
   optionally, a template RNA (or DNA encoding the template RNA) comprising (i) a sequence that binds the polypeptide and (ii) a heterologous object sequence; and
   an iPSC.
51. A method of modifying the genome of a mammalian respiratory epithelial cell (e.g., a bronchial epithelial cell, e.g., a human bronchial epithelial (hBE) cell), the method comprising contacting the cell with:
   (a) a gene modifying polypeptide, or a nucleic acid (e.g., DNA or mRNA) encoding the gene modifying polypeptide, and
   (b) a template RNA (or DNA encoding the template RNA) comprising (i) a sequence that binds the polypeptide and (ii) a heterologous object sequence.
52. The method of embodiment 51, which results in insertion of the heterologous object sequence into at least 5%, 10%, or 15% of respiratory epithelial cells (e.g., bronchial epithelial cells, e.g., human bronchial epithelial (hBE) cells).
53. A reaction mixture comprising:
   a gene modifying polypeptide, or a nucleic acid (e.g., DNA or mRNA) encoding the gene modifying polypeptide,
   optionally, a template RNA (or DNA encoding the template RNA) comprising (i) a sequence that binds the polypeptide and (ii) a heterologous object sequence; and
   a respiratory epithelial cell (e.g., a bronchial epithelial cell, e.g., a human bronchial epithelial (hBE) cell).
54. A gene modifying polypeptide comprising an amino acid sequence of SEQ ID NO: 403, or a sequence having at least 70%, 75%, 80%, 85%, 90%, 95%, 96%, 97%, 98%, or 99% identity thereto, wherein one or both of:
   i) amino acid position 345 is other than D, e.g., is N, and
   ii) amino acid position 523 is other than T, e.g., is S;
   or a functional fragment thereof (e.g., having one or both of reverse transcriptase activity and endonuclease activity).
55. A gene modifying polypeptide comprising an amino acid sequence of SEQ ID NO: 403, or a sequence having at least 70%, 75%, 80%, 85%, 90%, 95%, 96%, 97%, 98%, or 99% identity thereto, wherein one or both of:
   i) amino acid position 345 is N, and
   ii) amino acid position 523 is S;
   or a functional fragment thereof (e.g., having one or both of reverse transcriptase activity and endonuclease activity).
56. A gene modifying polypeptide comprising an amino acid sequence of SEQ ID NO: 405, or a sequence having at least 70%, 75%, 80%, 85%, 90%, 95%, 96%, 97%, 98%, or 99% identity thereto, wherein one, two, or three of:
   i) amino acid position 444 is other than P, e.g., is A,
   ii) amino acid position 848 is other than D, e.g., is G; and
   iii) amino acid position 875 is other than T, e.g., is A
   or a functional fragment thereof (e.g., having one or both of reverse transcriptase activity and endonuclease activity).
57. A gene modifying polypeptide comprising an amino acid sequence of SEQ ID NO: 405, or a sequence having at least 70%, 75%, 80%, 85%, 90%, 95%, 96%, 97%, 98%, or 99% identity thereto, wherein one, two, or three of:
   i) amino acid position 444 is A,
   ii) amino acid position 848 is G; and
   iii) amino acid position 875 is A
   or a functional fragment thereof (e.g., having one or both of reverse transcriptase activity and endonuclease activity).
58. A gene modifying polypeptide comprising an amino acid sequence of SEQ ID NO: 410, or a sequence having at least 70%, 75%, 80%, 85%, 90%, 95%, 96%, 97%, 98%, or 99% identity thereto, wherein one or both of:
   i) amino acid position 476 is a non-polar residue, e.g., is L, and
   ii) amino acid position 524 is a non-polar residue, e.g., is L;
   or a functional fragment thereof (e.g., having one or both of reverse transcriptase activity and endonuclease activity).
59. A gene modifying polypeptide comprising an amino acid sequence of SEQ ID NO: 410, or a sequence having at least 70%, 75%, 80%, 85%, 90%, 95%, 96%, 97%, 98%, or 99% identity thereto, wherein one or both of:
   i) amino acid position 476 is L, and
   ii) amino acid position 524 is L;
   or a functional fragment thereof (e.g., having one or both of reverse transcriptase activity and endonuclease activity).
60. A gene modifying polypeptide comprising an amino acid sequence of SEQ ID NO: 407, or a sequence having at least 70%, 75%, 80%, 85%, 90%, 95%, 96%, 97%, 98%, or 99% identity thereto, or a functional fragment thereof (e.g., having one or both of reverse transcriptase activity and endonuclease activity).
61. A gene modifying polypeptide comprising an amino acid sequence of SEQ ID NO: 408, or a sequence having at least 70%, 75%, 80%, 85%, 90%, 95%, 96%, 97%, 98%, or 99% identity thereto, or a functional fragment thereof (e.g., having one or both of reverse transcriptase activity and endonuclease activity).
62. A gene modifying polypeptide comprising an amino acid sequence of SEQ ID NO: 409, or a sequence having at least 70%, 75%, 80%, 85%, 90%, 95%, 96%, 97%, 98%, or 99% identity thereto, or a functional fragment thereof (e.g., having one or both of reverse transcriptase activity and endonuclease activity).
63. The method of any of embodiments 19, 20, 22-26, 35, 38, 48 or 49, wherein the DNA damage response (DDR) pathway in the cell (e.g., an iPSC) is not activated, or is activated less than in an otherwise similar cell treated with Cas9, e.g., in an assay according to Example 2.
64. The method of any of embodiments 19, 20, 22-26, 35, 38, 48, 49, or 63, wherein the interferon response is not activated, or is activated less than in an otherwise similar cell treated with a gene modifying system comprising elements from a LINE-1 retrotransposase, e.g., in an assay according to Example 3.
65. The system, method, cell, population of cells, or reaction mixture of any of the preceding embodiments, wherein the sequence that binds the polypeptide comprises a 5′ UTR, e.g., a 5′ UTR have a sequence listed in Table R1 or E14, or a sequence having at least 80%, 85%, 90%, 95%, 96%, 97%, 98%, or 99% identity thereto.
66. The system, method, cell, population of cells, or reaction mixture of any of the preceding embodiments, wherein the sequence that binds the polypeptide comprises a 3′ UTR, e.g., a 3′ UTR have a sequence listed in Table R1 or E14, or a sequence having at least 80%, 85%, 90%, 95%, 96%, 97%, 98%, or 99% identity thereto.
67. The system, method, cell, population of cells, or reaction mixture of any of the preceding embodiments, wherein:
   the sequence that binds the polypeptide comprises a 5′ UTR, e.g., a 5′ UTR have a sequence listed in Table R1 or E14, or a sequence having at least 70%, 75%, 80%, 85%, 90%, 95%, 96%, 97%, 98%, or 99% identity thereto, and
   the template RNA further comprises a 3′ UTR, e.g., a 3′ UTR have a sequence listed in Table R1 or E14, or a sequence having at least 70%, 75%, 80%, 85%, 90%, 95%, 96%, 97%, 98%, or 99% identity thereto.
68. The system, method, cell, population of cells, or reaction mixture of any of the preceding embodiments, wherein the template RNA further comprises one or both of:
   a 5′ target homology domain and
   a 3′ target homology domain.
69. The system, method, cell, population of cells, or reaction mixture of any of the preceding embodiments, wherein the gene modifying polypeptide comprises an amino acid sequence of Table R1 or E14 or a sequence at least 80%, 85%, 90%, 95%, 96%, 97%, 98%, or 99% identical thereto.
70. The system, method, cell, population of cells, or reaction mixture of any of the preceding embodiments, wherein the gene modifying polypeptide comprises an amino acid sequence of SEQ ID NO: 400 or a sequence at least 70%, 75%, 80%, 85%, 90%, 95%, 96%, 97%, 98%, or 99% identical thereto.
71. The system, method, cell, population of cells, or reaction mixture of any of the preceding embodiments, wherein the sequence that binds the polypeptide comprises:
   a 5′ UTR having a sequence of SEQ ID NO: 700 or a sequence at least 80%, 85%, 90%, 95%, 96%, 97%, 98%, or 99% identical thereto; and
   a 3′ UTR having a sequence of SEQ ID NO: 800 or a sequence at least 80%, 85%, 90%, 95%, 96%, 97%, 98%, or 99% identical thereto.
72. The system, method, cell, population of cells, or reaction mixture of any of the preceding embodiments, wherein the gene modifying polypeptide comprises an amino acid sequence of SEQ ID NO: 401 or a sequence at least 70%, 75%, 80%, 85%, 90%, 95%, 96%, 97%, 98%, or 99% identical thereto.
73. The system, method, cell, population of cells, or reaction mixture of embodiment 69, wherein the sequence that binds the polypeptide comprises:
   a 5′ UTR having a sequence of SEQ ID NO: 701 or a sequence at least 80%, 85%, 90%, 95%, 96%, 97%, 98%, or 99% identical thereto; and
   a 3′ UTR having a sequence of SEQ ID NO: 801 or a sequence at least 80%, 85%, 90%, 95%, 96%, 97%, 98%, or 99% identical thereto.
74. The system, method, cell, population of cells, or reaction mixture of any of the preceding embodiments, wherein the gene modifying polypeptide comprises an amino acid sequence of SEQ ID NO: 402 or a sequence at least 70%, 75%, 80%, 85%, 90%, 95%, 96%, 97%, 98%, or 99% identical thereto.
75. The system, method, cell, population of cells, or reaction mixture of embodiment 74, wherein the sequence that binds the polypeptide comprises:
   a 5′ UTR having a sequence of SEQ ID NO: 702 or a sequence at least 80%, 85%, 90%, 95%, 96%, 97%, 98%, or 99% identical thereto; and
   a 3′ UTR having a sequence of SEQ ID NO: 802 or a sequence at least 80%, 85%, 90%, 95%, 96%, 97%, 98%, or 99% identical thereto.
76. The system, method, cell, population of cells, or reaction mixture of any of the preceding embodiments, wherein the gene modifying polypeptide comprises an amino acid sequence of SEQ ID NO: 403 or a sequence at least 70%, 75%, 80%, 85%, 90%, 95%, 96%, 97%, 98%, or 99% identical thereto.
77. The system, method, cell, population of cells, or reaction mixture of embodiment 76, wherein the sequence that binds the polypeptide comprises:
   a 5′ UTR having a sequence of SEQ ID NO: 703 or a sequence at least 80%, 85%, 90%, 95%, 96%, 97%, 98%, or 99% identical thereto; and
   a 3′ UTR having a sequence of SEQ ID NO: 803 or a sequence at least 80%, 85%, 90%, 95%, 96%, 97%, 98%, or 99% identical thereto.
78. The system, method, cell, population of cells, or reaction mixture of any of the preceding embodiments, wherein the gene modifying polypeptide comprises an amino acid sequence of SEQ ID NO:404 or a sequence at least 70%, 75%, 80%, 85%, 90%, 95%, 96%, 97%, 98%, or 99% identical thereto.
79. The system, method, cell, population of cells, or reaction mixture of embodiment 78, wherein the sequence that binds the polypeptide comprises:
   a 5′ UTR having a sequence of SEQ ID NO: 704 or a sequence at least 80%, 85%, 90%, 95%, 96%, 97%, 98%, or 99% identical thereto; and
   a 3′ UTR having a sequence of SEQ ID NO: 804 or a sequence at least 80%, 85%, 90%, 95%, 96%, 97%, 98%, or 99% identical thereto.
80. The system, method, cell, population of cells, or reaction mixture of any of the preceding embodiments, wherein the gene modifying polypeptide comprises an amino acid sequence of SEQ ID NO: 405 or a sequence at least 70%, 75%, 80%, 85%, 90%, 95%, 96%, 97%, 98%, or 99% identical thereto.
81. The system, method, cell, population of cells, or reaction mixture of embodiment 80, wherein the sequence that binds the polypeptide comprises:
   a 5′ UTR having a sequence of SEQ ID NO: 705 or a sequence at least 80%, 85%, 90%, 95%, 96%, 97%, 98%, or 99% identical thereto; and
   a 3′ UTR having a sequence of SEQ ID NO: 805 or a sequence at least 80%, 85%, 90%, 95%, 96%, 97%, 98%, or 99% identical thereto.
82. The system, method, cell, population of cells, or reaction mixture of any of the preceding embodiments, wherein the gene modifying polypeptide comprises an amino acid sequence of SEQ ID NO: 406 or a sequence at least 70%, 75%, 80%, 85%, 90%, 95%, 96%, 97%, 98%, or 99% identical thereto.
83. The system, method, cell, population of cells, or reaction mixture of embodiment 82, wherein the sequence that binds the polypeptide comprises:
   a 5′ UTR having a sequence of SEQ ID NO: 706 or a sequence at least 80%, 85%, 90%, 95%, 96%, 97%, 98%, or 99% identical thereto; and
   a 3′ UTR having a sequence of SEQ ID NO: 806 or a sequence at least 80%, 85%, 90%, 95%, 96%, 97%, 98%, or 99% identical thereto.
84. The system, method, cell, population of cells, or reaction mixture of any of the preceding embodiments, wherein the gene modifying polypeptide comprises an amino acid sequence of SEQ ID NO: 407 or a sequence at least 70%, 75%, 80%, 85%, 90%, 95%, 96%, 97%, 98%, or 99% identical thereto.
85. The system, method, cell, population of cells, or reaction mixture of embodiment 84, wherein the sequence that binds the polypeptide comprises:
   a 5′ UTR having a sequence of SEQ ID NO: 707 or a sequence at least 80%, 85%, 90%, 95%, 96%, 97%, 98%, or 99% identical thereto; and
   a 3′ UTR having a sequence of SEQ ID NO: 807 or a sequence at least 80%, 85%, 90%, 95%, 96%, 97%, 98%, or 99% identical thereto.
86. The system, method, cell, population of cells, or reaction mixture of any of the preceding embodiments, wherein the gene modifying polypeptide comprises an amino acid sequence of SEQ ID NO: 408 or a sequence at least 70%, 75%, 80%, 85%, 90%, 95%, 96%, 97%, 98%, or 99% identical thereto.
87. The system, method, cell, population of cells, or reaction mixture of embodiment 86, wherein the sequence that binds the polypeptide comprises:
   a 5′ UTR having a sequence of SEQ ID NO: 708 or a sequence at least 80%, 85%, 90%, 95%, 96%, 97%, 98%, or 99% identical thereto; and
   a 3′ UTR having a sequence of SEQ ID NO: 808 or a sequence at least 80%, 85%, 90%, 95%, 96%, 97%, 98%, or 99% identical thereto.
88. The system, method, cell, population of cells, or reaction mixture of any of the preceding embodiments, wherein the gene modifying polypeptide comprises an amino acid sequence of SEQ ID NO: 409 or a sequence at least 70%, 75%, 80%, 85%, 90%, 95%, 96%, 97%, 98%, or 99% identical thereto.
89. The system, method, cell, population of cells, or reaction mixture of embodiment 88, wherein the sequence that binds the polypeptide comprises:
   a 5′ UTR having a sequence of SEQ ID NO: 709 or a sequence at least 80%, 85%, 90%, 95%, 96%, 97%, 98%, or 99% identical thereto; and
   a 3′ UTR having a sequence of SEQ ID NO: 809 or a sequence at least 80%, 85%, 90%, 95%, 96%, 97%, 98%, or 99% identical thereto.
90. The system, method, cell, population of cells, or reaction mixture of any of the preceding embodiments, wherein the gene modifying polypeptide comprises an amino acid sequence of SEQ ID NO: 410 or a sequence at least 70%, 75%, 80%, 85%, 90%, 95%, 96%, 97%, 98%, or 99% identical thereto.
91. The system, method, cell, population of cells, or reaction mixture of embodiment 90, wherein the sequence that binds the polypeptide comprises:
   a 5′ UTR having a sequence of SEQ ID NO: 710 or a sequence at least 80%, 85%, 90%, 95%, 96%, 97%, 98%, or 99% identical thereto; and
   a 3′ UTR having a sequence of SEQ ID NO: 810 or a sequence at least 80%, 85%, 90%, 95%, 96%, 97%, 98%, or 99% identical thereto.
92. A pharmaceutical composition comprising the system of any of embodiments 1-4 or 65-91.
93. A lipid nanoparticle (LNP) composition comprising the system of any of embodiments 1-4 or 65-91.
94. The LNP composition of embodiment 93, wherein (a) and (b) are encapsulated in the same LNP.
95. The LNP composition of embodiment 93, wherein (a) and (b) are encapsulated in different LNPs.
96. A method for modifying the genome of a mammalian cell, the method comprising contacting a population of cells with:
   (a) a heterologous gene modifying system comprising:
      (i) a heterologous gene modifying polypeptide or a nucleic acid (e.g., DNA or mRNA) encoding the heterologous gene modifying polypeptide,
      (ii) a first template RNA (or DNA encoding the template RNA) comprising (1) a gRNA spacer, (2) a gRNA scaffold, (3) a heterologous object sequence, and (4) a primer binding site (PBS) sequence, wherein the first template RNA is configured to produce a first mutation; and
      (iii) a second template RNA (or DNA encoding the template RNA) comprising (1) a gRNA spacer, (2) a gRNA scaffold, (3) a heterologous object sequence, and (4) a primer binding site (PBS) sequence, wherein the second template RNA is configured to produce a second mutation; and
   (b) a retrotransposon gene modifying system comprising:
      (i) a retrotransposon gene modifying polypeptide or a nucleic acid (e.g., DNA or mRNA) encoding the retrotransposon gene modifying polypeptide, and
      (ii) a template RNA (or DNA encoding the template RNA) comprising (1) a sequence that binds the polypeptide and (2) a heterologous object sequence encoding a transgene.
97. The method of embodiment 96, which results in installation of the first and second mutations (e.g., insertions) in at least 40%, 50%, 60%, 70%, 75% of said cells.
98. The method of embodiment 96 or 97, which results in installation of the transgene in at least 5%, 10%, 15% of said cells.
99. The method of any of embodiments 96-98, which results in installation of the first mutation, the second mutation, and the transgene in at least 5%, 10%, 15% of said cells.

### Definitions

Antigen binding domain: The term “antigen binding domain” as used herein refers to that portion of antibody or a chimeric antigen receptor which binds an antigen. In some embodiments, an antigen binding domain binds to a cell surface antigen of a cell. In some embodiments an antigen binding domain binds an antigen characteristic of a cancer, e.g., a tumor associated antigen in a neoplastic cell. In some embodiments, an antigen binding domain binds an antigen characteristic of an infectious disease, e.g. a virus associated antigen in a virus infected cell. In some embodiments, an antigen binding domain binds an antigen characteristic of a cell targeted by a subject's immune system in an autoimmune disease, e.g., a self-antigen. In some embodiments, an antigen binding domain is or comprises an antibody or antigen-binding portion thereof. In some embodiments, an antigen binding domain is or comprises an scFv, Fab, diabody, D domain binder, centryin, or one or more single domain antibodies (e.g., VHH domains)

Domain: The term “domain” as used herein refers to a structure of a biomolecule that contributes to a specified function of the biomolecule. A domain may comprise a contiguous region (e.g., a contiguous sequence) or distinct, non-contiguous regions (e.g., non-contiguous sequences) of a biomolecule. Examples of protein domains include, but are not limited to, an endonuclease domain, a DNA binding domain, a reverse transcriptase domain; an example of a domain of a nucleic acid is a regulatory domain, such as a transcription factor binding domain.

Exogenous: As used herein, the term “exogenous,” when used with reference to a biomolecule (such as a nucleic acid sequence or polypeptide) means that the biomolecule was introduced into a host genome, cell, or organism by the hand of man. For example, a nucleic acid that is as added into an existing genome, cell, tissue, or subject using recombinant DNA techniques or other methods is exogenous to the existing nucleic acid sequence, cell, tissue or subject.

Genomic safe harbor site (GSH site): A “genomic safe harbor site” is a site in a host genome that is able to accommodate the integration of new genetic material, e.g., such that the inserted genetic element does not cause significant alterations of the host genome posing a risk to the host cell or organism. A GSH site generally meets 1, 2, 3, 4, 5, 6, 7, 8 or 9 of the following criteria: (i) is located >300 kb from a cancer-related gene; (ii) is >300 kb from a miRNA/other functional small RNA; (iii) is >50 kb from a 5′ gene end; (iv) is >50 kb from a replication origin; (v) is >50 kb away from any ultraconservered element; (vi) has low transcriptional activity (i.e. no mRNA+/−25 kb); (vii) is not in copy number variable region; (viii) is in open chromatin; and/or (ix) is unique, with 1 copy in the human genome. Examples of GSH sites in the human genome that meet some or all of these criteria include (i) the adeno-associated virus site 1 (AAVS1), a naturally occurring site of integration of AAV virus on chromosome 19; (ii) the chemokine (C-C motif) receptor 5 (CCR5) gene, a chemokine receptor gene known as an HIV-1 coreceptor; (iii) the human ortholog of the mouse Rosa26 locus; (iv) the rDNA locus. Additional GSH sites are known and described, e.g., in Pellenz et al. epub Aug. 20, 2018 (doi.org/10.1101/396390).

Heterologous: The term “heterologous”, when used to describe a first element in reference to a second element means that the first element and second element do not exist in nature disposed as described. For example, a heterologous polypeptide, nucleic acid molecule, construct or sequence refers to (a) a polypeptide, nucleic acid molecule or portion of a polypeptide or nucleic acid molecule sequence that is not native to a cell in which it is expressed, (b) a polypeptide or nucleic acid molecule or portion of a polypeptide or nucleic acid molecule that has been altered or mutated relative to its native state, or (c) a polypeptide or nucleic acid molecule with an altered expression as compared to the native expression levels under similar conditions. For example, a heterologous regulatory sequence (e.g., promoter, enhancer) may be used to regulate expression of a gene or a nucleic acid molecule in a way that is different than the gene or a nucleic acid molecule is normally expressed in nature. In another example, a heterologous domain of a polypeptide or nucleic acid sequence (e.g., a DNA binding domain of a polypeptide or nucleic acid encoding a DNA binding domain of a polypeptide) may be disposed relative to other domains or may be a different sequence or from a different source, relative to other domains or portions of a polypeptide or its encoding nucleic acid. In certain embodiments, a heterologous nucleic acid molecule may exist in a native host cell genome, but may have an altered expression level or have a different sequence or both. In other embodiments, heterologous nucleic acid molecules may not be endogenous to a host cell or host genome but instead may have been introduced into a host cell by transformation (e.g., transfection, electroporation), wherein the added molecule may integrate into the host genome or can exist as extra-chromosomal genetic material either transiently (e.g., mRNA) or semi-stably for more than one generation (e.g., episomal viral vector, plasmid or other self-replicating vector). In some embodiments, a domain is heterologous relative to another domain, if the first domain is not naturally comprised in the same polypeptide as the other domain (e.g., a fusion between two domains of different proteins from the same organism).

Mutation or Mutated: The term “mutated” when applied to nucleic acid sequences means that nucleotides in a nucleic acid sequence may be inserted, deleted or changed compared to a reference (e.g., native) nucleic acid sequence. A single alteration may be made at a locus (a point mutation) or multiple nucleotides may be inserted, deleted, or changed at a single locus. In addition, one or more alterations may be made at any number of loci within a nucleic acid sequence. A nucleic acid sequence may be mutated by any method known in the art. In some embodiments a mutation occurs naturally. In some embodiments a desired mutation can be produced by a system described herein.

Nucleic acid molecule: “Nucleic acid molecule” refers to both RNA and DNA molecules including, without limitation, cDNA, genomic DNA and mRNA, and also includes synthetic nucleic acid molecules, such as those that are chemically synthesized or recombinantly produced, such as RNA templates, as described herein. The nucleic acid molecule can be double-stranded or single-stranded, circular or linear. If single-stranded, the nucleic acid molecule can be the sense strand or the antisense strand. Unless otherwise indicated, and as an example for all sequences described herein under the general format “SEQ ID NO:,” “nucleic acid comprising SEQ ID NO:1” refers to a nucleic acid, at least a portion which has either (i) the sequence of SEQ ID NO:1, or (ii) a sequence complimentary to SEQ ID NO:1. The choice between the two is dictated by the context in which SEQ ID NO:1 is used. For instance, if the nucleic acid is used as a probe, the choice between the two is dictated by the requirement that the probe be complimentary to the desired target. Nucleic acid sequences of the present disclosure may be modified chemically or biochemically or may contain non-natural or derivatized nucleotide bases, as will be readily appreciated by those of skill in the art. Such modifications include, for example, labels, methylation, substitution of one or more naturally occurring nucleotides with an analog, inter-nucleotide modifications such as uncharged linkages (for example, methyl phosphonates, phosphotriesters, phosphoramidates, carbamates, etc.), charged linkages (for example, phosphorothioates, phosphorodithioates, etc.), pendant moieties, (for example, polypeptides), intercalators (for example, acridine, psoralen, etc.), chelators, alkylators, and modified linkages (for example, alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of a molecule. Other modifications can include, for example, analogs in which the ribose ring contains a bridging moiety or other structure such as modifications found in “locked” nucleic acids.

Gene expression unit: a “gene expression unit” is a nucleic acid sequence comprising at least one regulatory nucleic acid sequence operably linked to at least one effector sequence. A first nucleic acid sequence is operably linked with a second nucleic acid sequence when the first nucleic acid sequence is placed in a functional relationship with the second nucleic acid sequence. For instance, a promoter or enhancer is operably linked to a coding sequence if the promoter or enhancer affects the transcription or expression of the coding sequence. Operably linked DNA sequences may be contiguous or non-contiguous. Where necessary to join two protein-coding regions, operably linked sequences may be in the same reading frame.

Gene modifying polypeptide: A “gene modifying polypeptide,” and “retrotransposon gene modifying polypeptide” as used herein interchangeably to refer to a polypeptide comprising a retrotransposase reverse transcriptase domain and a retrotransposase endonuclease domain, or a polypeptide comprising an amino acid sequence having at least 75%, 80%, 85%, 90%, 95%, 96%, 97%, 98%, or 99% amino acid sequence identity to said domains, which is capable of integrating a nucleic acid sequence (e.g., a sequence provided on a template nucleic acid) into a target DNA molecule (e.g., in a mammalian host cell, such as a genomic DNA molecule in the host cell). In some embodiments, the endonuclease domain is a catalytically inactive endonuclease domain. In some embodiments, the retrotransposase reverse transcriptase domain and a retrotransposase endonuclease domain are derived from the same retrotransposase. In some embodiments, the gene modifying polypeptide is capable of integrating the sequence substantially without relying on host machinery. In some embodiments, the gene modifying polypeptide integrates a sequence into a random position in a genome, and in some embodiments, the gene modifying polypeptide integrates a sequence into a specific target site. In some embodiments, a gene modifying polypeptide includes one or more domains that, collectively, facilitate 1) binding the template nucleic acid, 2) binding the target DNA molecule, and 3) facilitate integration of the at least a portion of the template nucleic acid into the target DNA. Gene modifying polypeptides include both naturally occurring polypeptides as well as engineered variants of the foregoing, e.g., having one or more amino acid substitutions to the naturally occurring sequence. Gene modifying polypeptides also include heterologous constructs, e.g., where one or more of the domains recited above are heterologous to each other, whether through a heterologous fusion (or other conjugate) of otherwise wild-type domains, as well as fusions of modified domains, e.g., by way of replacement or fusion of a heterologous sub-domain or other substituted domain. Exemplary gene modifying polypeptides, and systems comprising them and methods of using them, that can be used in the methods provided herein are described, e.g., in WO/2021/178717, which is incorporated herein by reference, including Tables 10, 11, X, 3A, 3B, and Z1 therein. In some embodiments, a gene modifying polypeptide integrates a sequence into a gene. In some embodiments, a gene modifying polypeptide integrates a sequence into a sequence outside of a gene. A “gene modifying system,” as used herein, refers to a system comprising a gene modifying polypeptide and a template nucleic acid.

Heterologous gene modifying polypeptide: As used herein, the term “heterologous gene modifying polypeptide” refers to a polypeptide comprising a retroviral reverse transcriptase, or a polypeptide comprising an amino acid sequence having at least 75%, 80%, 85%, 90%, 95%, 96%, 97%, 98%, or 99% amino acid sequence identity to a retroviral reverse transcriptase, which is capable of integrating a nucleic acid sequence (e.g., a sequence provided on a template nucleic acid) into a target DNA molecule (e.g., in a mammalian host cell, such as a genomic DNA molecule in the host cell). In some embodiments, the heterologous gene modifying polypeptide is capable of integrating the sequence substantially without relying on host machinery. In some embodiments, the heterologous gene modifying polypeptide integrates a sequence into a random position in a genome, and in some embodiments, the heterologous gene modifying polypeptide integrates a sequence into a specific target site. In some embodiments, the sequence that is integrated comprises a deletion, substitution, or insertion relative to the target DNA molecule. In some embodiments, a heterologous gene modifying polypeptide includes one or more domains that, collectively, facilitate 1) binding the template nucleic acid, 2) binding the target DNA molecule, and 3) facilitate integration of the at least a portion of the template nucleic acid into the target DNA. Heterologous gene modifying polypeptides include both naturally occurring polypeptides as well as engineered variants of the foregoing, e.g., having one or more amino acid substitutions to the naturally occurring sequence. Heterologous gene modifying polypeptides also include heterologous constructs, e.g., where one or more of the domains recited above are heterologous to each other, whether through a heterologous fusion (or other conjugate) of otherwise wild-type domains, as well as fusions of modified domains, e.g., by way of replacement or fusion of a heterologous sub-domain or other substituted domain. Exemplary heterologous gene modifying polypeptides, and systems comprising them and methods of using them, that can be used in the methods provided herein are described, e.g., in PCT/US2021/020948, which is incorporated herein by reference with respect to heterologous gene modifying polypeptides that comprise a retroviral reverse transcriptase domain. In some embodiments, a heterologous gene modifying polypeptide integrates a sequence into a gene. In some embodiments, a heterologous gene modifying polypeptide integrates a sequence into a sequence outside of a gene.

Host: The terms “host genome” or “host cell,” as used herein, refer to a cell and/or its genome into which protein and/or genetic material has been introduced. It should be understood that such terms are intended to refer not only to the particular subject cell and/or genome, but to the progeny of such a cell and/or the genome of the progeny of such a cell. Because certain modifications may occur in succeeding generations due to either mutation or environmental influences, such progeny may not, in fact, be identical to the parent cell, but are still included within the scope of the term “host cell” as used herein. A host genome or host cell may be an isolated cell or cell line grown in culture, or genomic material isolated from such a cell or cell line, or may be a host cell or host genome which composing living tissue or an organism. In some instances, a host cell may be an animal cell or a plant cell, e.g., as described herein. In certain instances, a host cell may be a bovine cell, horse cell, pig cell, goat cell, sheep cell, chicken cell, or turkey cell. In certain instances, a host cell may be a corn cell, soy cell, wheat cell, or rice cell.

Pseudoknot: A “pseudoknot sequence” sequence, as used herein, refers to a nucleic acid (e.g., RNA) having a sequence with suitable self-complementarity to form a pseudoknot structure, e.g., having: a first segment, a second segment between the first segment and a third segment, wherein the third segment is complementary to the first segment, and a fourth segment, wherein the fourth segment is complementary to the second segment. The pseudoknot may optionally have additional secondary structure, e.g., a stem loop disposed in the second segment, a stem-loop disposed between the second segment and third segment, sequence before the first segment, or sequence after the fourth segment. The pseudoknot may have additional sequence between the first and second segments, between the second and third segments, or between the third and fourth segments. In some embodiments, the segments are arranged, from 5′ to 3′: first, second, third, and fourth. In some embodiments, the first and third segments comprise five base pairs of perfect complementarity. In some embodiments, the second and fourth segments comprise 10 base pairs, optionally with one or more (e.g., two) bulges. In some embodiments, the second segment comprises one or more unpaired nucleotides, e.g., forming a loop. In some embodiments, the third segment comprises one or more unpaired nucleotides, e.g., forming a loop.

Stem-loop sequence: As used herein, a “stem-loop sequence” refers to a nucleic acid sequence (e.g., RNA sequence) with sufficient self-complementarity to form a stem-loop, e.g., having a stem comprising at least two (e.g., 3, 4, 5, 6, 7, 8, 9, or 10) base pairs, and a loop with at least three (e.g., four) base pairs. The stem may comprise mismatches or bulges.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic of a gene modifying system.

FIGS. 2A-2B are a series of charts demonstrating that a gene modifying systems described herein yield a different integration profile than a lentiviral system.

FIGS. 3A-3B are a series of graphs showing integration and expression of a template in primary cells by gene modifying systems delivered as all RNA (FIG. 3A) and a pair of blots demonstrating that a gene modifying system described herein does not activate DNA damage response pathways in primary cells (FIG. 3B).

FIG. 4 is a pair of graphs demonstrating that a gene modifying system described herein do not activate interferon response in primary cells.

FIG. 5 is a pair of graphs demonstrating the effects of a point mutation at the endonuclease active site on retrotransposition activity in human cells. (A) Retrotransposition as measured by a GFPai reporter. (B) Retrotransposition as measured by ddPCR assay.

FIGS. 6A-6C demonstrates that gene editing of a primary human T cells to install a BCMA CAR by a gene modifying system results in CART cells that can kill target tumor cells. FIG. 6A is a diagram showing the elements of the CAR molecule. FIG. 6B is a series of flow cytometry plots showing the percentage of CAR+ T cells. FIG. 6C is a graph showing the % killing of tumor cells when contacted with CART cells produced with retrotransposon-based gene modifying systems and lentiviral systems.

FIG. 7 is a graph showing expression of BCMA-CAR from primary human T cells electroporated with RTE1_MD gene modifying polypeptide encoding nucleic acid and BCMA CAR template. As controls, T cells were electroporated with only RTE1_MD gene modifying polypeptide encoding nucleic acid and only with BCMA-CAR template. Each data point represents one donor.

FIGS. 8A and 8B are graphs showing cytotoxic killing of RTE1_MD gene modifying system-derived BCMA CART cells from two different donors (FIG. 8A) and IFNgamma production and Granzyme B release from supernatants (FIG. 8A) as measured by ELISA (FIG. 8B).

FIG. 9A is a process flow for introducing Vingi-1_Acar gene modifying system to activated human primary T cells.

FIG. 9B is a schematic of BCMA-CAR-T2A-RQR8 template used (top panel) and a graph showing percentage of primary human T cells expressing BCMA-CAR before and after CD34 bead-based enrichment (bottom panel).

FIGS. 10A and 10B are graphs showing percent killing (FIG. 10A) and IFNγ levels (FIG. 10B) following co-culture of BCMA-CAR T cells with BCMA-positive tumor cell lines.

FIGS. 11A-11C are graphs showing individual tumor volume following treatment with vehicle (FIG. 11A), untransduced T cells (FIG. 11B), or BCMA CART (FIG. 11C).

FIG. 12 is a graph showing levels of editing by the second gene modifying system in the bulk T cell population when the cells were transfected with the second gene modifying system, either alone or in combination with the first gene modifying system.

FIG. 13 is a graph showing the levels of editing by the first gene modifying system in the bulk T cell population when the cells were transfected with the first gene modifying system, either alone or in combination with the second gene modifying system.

FIG. 14 is a graph showing shows the levels of editing by the first gene modifying system (measured here as loss of expression of CD3) when the cells were co-transfected with the first gene modifying system specifically breaking out TRAC editing levels in sub-populations of cells that were edited or not edited by the second gene modifying system (that were GFP+ or BCMA CAR+ T cells, or GFP− or BCMA CAR−).

FIG. 15 is a graph showing a phenotypic characterization by flow cytometry of T cells treated with the first gene modifying system, both the first and second gene modifying systems, or a mock treatment lacking both gene modifying systems.

FIG. 16 is a graph showing expression of BCMA-CAR from primary human T cells electroporated with Vingi1-Acar gene modifying polypeptide and a BCMA CAR template or RTE1_MD gene modifying polypeptide and BCMA CAR template.

FIG. 17 is a graph showing the levels of editing by the retrotransposon gene modifying system in the bulk T cell population when the cells were transfected with the retrotransposon gene modifying system, either alone or in combination with the heterologous gene modifying system.

FIG. 18 is a graph showing the levels of editing by the heterologous gene modifying system (containing a template RNA designed to produce an insertion in TRAC and a template RNA designed to produce an insertion in B2M) in the bulk T cell population when the cells were transfected with the heterologous gene modifying system either alone or in combination with the retrotransposon gene modifying system.

FIG. 19 is a graph showing the levels of cells edited by the heterologous gene modifying system (measured here as combined loss of expression of CD3 and B2M by flow cytometry) and also edited by the retrotransposon gene editing system (that were GFP+ or BCMA CAR+ T cells).

FIG. 20 is a graph showing a phenotypic characterization by flow cytometry of T cells treated with the heterologous gene modifying system (containing a template RNA designed to produce an insertion in TRAC and a template RNA designed to produce an insertion in B2M), both the heterologous and retrotransposon gene modifying systems, or a mock treatment lacking both gene modifying systems.

FIG. 21 is a graph showing percent cytokine expressing cells as assessed by flow cytometry of T cells edited with the heterologous gene modifying system (containing a template RNA designed to produce an insertion in TRAC and a template RNA designed to produce an insertion in B2M), edited with both the heterologous and retrotransposon gene modifying systems, or a mock treated cells edited by neither gene modifying systems.

FIG. 22 is a graph showing the percentage of edited activated T cells at the TRAC and B2M loci by a first gene modifying system comprising a WT Cas9-RT fusion polypeptide and second heterologous gene modifying system comprising an exemplary heterologous gene modifying polypeptide.

FIG. 23 is a graph showing percent translocation in T cells following gene editing at the TRAC and B2M loci by a first gene modifying system comprising a WT Cas9-RT fusion polypeptide and second heterologous gene modifying system comprising an exemplary heterologous gene modifying polypeptide.

FIG. 24 is a graph showing integration and expression of a template in primary cells and in iPSCs by gene modifying systems delivered as all RNA.

FIG. 25 is pair of graphs showing BCMA CAR expression in human T cells (left) following transfection with an RTE-1 MD gene modifying system and template encoding a CAR and the % killing of tumor cells (right) when contacted with CART cells.

FIG. 26 is a graph showing CD20 CAR expression in human T cells following transfection with an RTE-1 MD gene modifying polypeptide and CD20 CAR template.

FIG. 27 is a graph showing expression of GFP, a CAR, or both in human T cells following transfection with a Vingi-1_Acar gene modifying polypeptide and a template encoding GFP, a template encoding a CAR, or both templates.

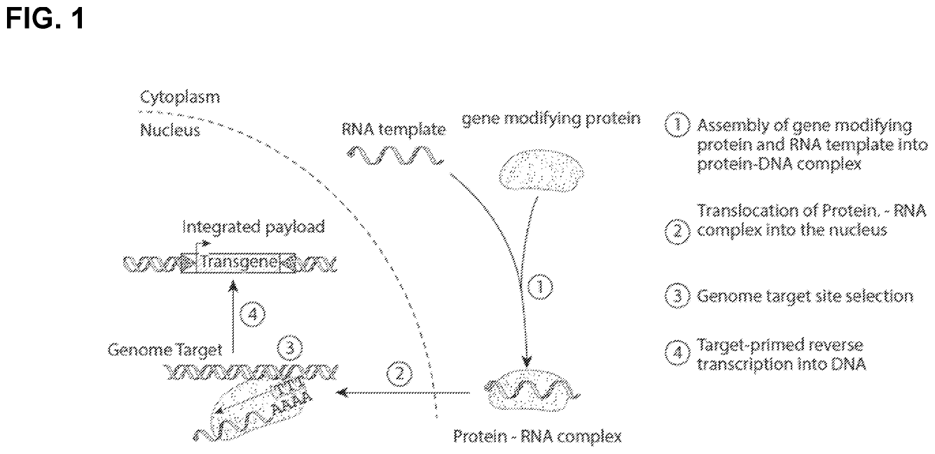

### DETAILED DESCRIPTION

This disclosure relates to compositions, systems and methods for targeting, editing, modifying or manipulating a DNA sequence (e.g., inserting a heterologous object DNA sequence into a target site of a mammalian genome) at one or more locations in a DNA sequence in a cell, tissue or subject, e.g., in vivo, in vitro or ex vivo. The object DNA sequence may include, e.g., a coding sequence, a regulatory sequence, a gene expression unit.

More specifically, the disclosure provides retrotransposon-based systems for inserting a sequence of interest into the genome. Examples of retrotransposon elements are listed, e.g., in Tables 10, 11, X, 3A, 3B, and Z1 of PCT Publication No. WO/2021/178717, incorporated herein by reference in its entirety.

In some embodiments, systems described herein can have a number of advantages relative to various earlier systems. For instance, the disclosure describes retrotransposases capable of inserting long sequences of heterologous nucleic acid into a genome. In addition, retrotransposases described herein can insert heterologous nucleic acid in an endogenous site in the genome, such as the rDNA locus. This is in contrast to Cre/loxP systems, which require a first step of inserting an exogenous loxP site before a second step of inserting a sequence of interest into the loxP site.

### Gene Modifying Polypeptides

Non-long terminal repeat (LTR) retrotransposons are a type of mobile genetic elements that are widespread in eukaryotic genomes. They include, for example, the apurinic/apyrimidinic endonuclease (APE)-type, the restriction enzyme-like endonuclease (RLE)-type, and the Penelope-like element (PLE)-type.

The APE class retrotransposons are comprised of two functional domains: an endonuclease/DNA binding domain, and a reverse transcriptase domain. Examples of APE-class retrotransposons can be found, for example, in Table 1 of PCT Application No. PCT/US2019/048607, incorporated herein by reference in its entirety, including the sequence listing and sequences referred to in Table 1 therein.

The RLE class are comprised of three functional domains: a DNA binding domain, a reverse transcription domain, and an endonuclease domain. Examples of RLE-class retrotransposons can be found, for example, in Table 2 of PCT Application No. PCT/US2019/048607, incorporated herein by reference in its entirety, including the sequence listing and sequences referred to in Table 2 therein.

The reverse transcriptase domain of non-LTR retrotransposon functions by binding an RNA sequence template and reverse transcribing it into the host genome's target DNA. The RNA sequence template has a 3′ untranslated region which is specifically bound to the retrotransposase, and a variable 5′ region generally having Open Reading Frame(s) (“ORF”) encoding retrotransposase proteins. The RNA sequence template may also comprise a 5′ untranslated region which specifically binds the retrotransposase.

Penelope-like elements (PLEs) are distinct from both LTR and non-LTR retrotransposons. PLEs generally comprise a reverse transcriptase domain distinct from that of APE and RLE elements, but similar to that of telomerases and Group II introns, and an optional GIY-YIG endonuclease domain.

Other exemplary classes of retrotransposon include, without limitation, RTE (e.g., RTE-1_MD, RTE-3_BF, and RTE-25_LMi), CR1 (e.g., CR1-1_PH), Crack (e.g., Crack-28_RF), L2 (e.g., L2-2_Dre and L2-5_GA), and Vingi (e.g., Vingi-1_Acar) retrotransposons.

As described herein, the elements of such retrotransposons can be functionally modularized and/or modified to target, edit, modify or manipulate a target DNA sequence, e.g., to insert an object (e.g., heterologous) nucleic acid sequence into a target genome, e.g., a mammalian genome, by reverse transcription. In some embodiments, a gene modifying system comprises: (A) a polypeptide or a nucleic acid encoding a polypeptide, wherein the polypeptide comprises (i) a retrotransposase reverse transcriptase domain, and (ii) a retrotransposase endonuclease domain that contains DNA binding functionality; and (B) a template RNA (or DNA encoding the template RNA) comprising (i) a sequence that binds the polypeptide and (ii) a heterologous object sequence. The RNA template element of a gene modifying system is typically heterologous to the polypeptide element and provides an object sequence to be inserted (reverse transcribed) into the host genome.

In some embodiments, the gene modifying system comprises a retrotransposase sequence of an element listed in any one of Table 10, Table 11, Table X, Table Z1 Table 3A, or 3B of PCT Pub. No.: WO/2021/178717, which are incorporated herein by reference as they relate to domains from retrotransposons.

In some embodiments, an amino acid sequence encoded by an element of Table R1 is an amino acid sequence encoded by the full length sequence of an element listed in Table R1, or a sequence having at least 75%, 80%, 85%, 90%, 95%, 96%, 97%, 98%, or 99% sequence identity thereto. In some embodiments, the full-length sequence of an element listed in Table R1 may comprise one or more (e.g., all of) of a 5′ UTR, polypeptide-encoding sequence, or 3′ UTR of a retrotransposon as described herein. In some embodiments, an amino acid sequence of Table R1 is an amino acid sequence encoded by the full length sequence of an element listed in Table R1, or a sequence having at least 75%, 80%, 85%, 90%, 95%, 96%, 97%, 98%, or 99% sequence identity thereto. In some embodiments, a 5′ UTR of an element of Table R1 comprises a 5′ UTR of the full length sequence of an element listed in Table R1, or a sequence having at least 75%, 80%, 85%, 90%, 95%, 96%, 97%, 98%, or 99% sequence identity thereto. In some embodiments, a 3′ UTR of an element of Table R1 comprises a 3′ UTR of the full length sequence of an element listed in Table R1, or a sequence having at least 75%, 80%, 85%, 90%, 95%, 96%, 97%, 98%, or 99% sequence identity thereto.

Also indicated in Table R1 are the host organisms from which the nucleic acid sequences were obtained and a listing of domains present within the polypeptide encoded by the open reading frame of the nucleic acid sequence.

In some embodiments, the gene modifying polypeptide comprises an amino acid sequence of SEQ ID NO: 400 or a sequence at least 70%, 75%, 80%, 85%, 90%, 95%, 96%, 97%, 98%, or 99% identical thereto, or a functional fragment thereof (e.g., having one or both of reverse transcriptase activity and endonuclease activity). In some embodiments, the gene modifying polypeptide comprises an amino acid sequence of SEQ ID NO: 400. In some embodiments, the sequence that binds the polypeptide comprises:
a 5′ UTR having a sequence of SEQ ID NO: 700 or a sequence at least 80%, 85%, 90%, 95%, 96%, 97%, 98%, or 99% identical thereto; and
a 3′ UTR having a sequence of SEQ ID NO: 800 or a sequence at least 80%, 85%, 90%, 95%, 96%, 97%, 98%, or 99% identical thereto.

In some embodiments, the gene modifying polypeptide comprises an amino acid sequence of SEQ ID NO: 401 or a sequence at least 70%, 75%, 80%, 85%, 90%, 95%, 96%, 97%, 98%, or 99% identical thereto, or a functional fragment thereof (e.g., having one or both of reverse transcriptase activity and endonuclease activity). In some embodiments, the gene modifying polypeptide comprises an amino acid sequence of SEQ ID NO: 401. In some embodiments, the sequence that binds the polypeptide comprises:
a 5′ UTR having a sequence of SEQ ID NO: 701 or a sequence at least 80%, 85%, 90%, 95%, 96%, 97%, 98%, or 99% identical thereto; and
a 3′ UTR having a sequence of SEQ ID NO: 801 or a sequence at least 80%, 85%, 90%, 95%, 96%, 97%, 98%, or 99% identical thereto.

In some embodiments, the gene modifying polypeptide comprises an amino acid sequence of SEQ ID NO: 402 or a sequence at least 70%, 75%, 80%, 85%, 90%, 95%, 96%, 97%, 98%, or 99% identical thereto, or a functional fragment thereof (e.g., having one or both of reverse transcriptase activity and endonuclease activity). In some embodiments, the gene modifying polypeptide comprises an amino acid sequence of SEQ ID NO: 402. In some embodiments, the sequence that binds the polypeptide comprises:
a 5′ UTR having a sequence of SEQ ID NO: 702 or a sequence at least 80%, 85%, 90%, 95%, 96%, 97%, 98%, or 99% identical thereto; and
a 3′ UTR having a sequence of SEQ ID NO: 802 or a sequence at least 80%, 85%, 90%, 95%, 96%, 97%, 98%, or 99% identical thereto.

In some embodiments, the gene modifying polypeptide comprises an amino acid sequence of SEQ ID NO: 403, or a sequence having at least 70%, 75%, 80%, 85%, 90%, 95%, 96%, 97%, 98%, or 99% identity thereto, wherein one or both of:
i) amino acid position 345 is other than D, e.g., is N, and
ii) amino acid position 523 is other than T, e.g., is S;
or a functional fragment thereof (e.g., having one or both of reverse transcriptase activity and endonuclease activity).

In some embodiments, the gene modifying polypeptide comprises an amino acid sequence of SEQ ID NO: 403, or a sequence having at least 70%, 75%, 80%, 85%, 90%, 95%, 96%, 97%, 98%, or 99% identity thereto, wherein one or both of:
i) amino acid position 345 is N, and
ii) amino acid position 523 is S;
or a functional fragment thereof (e.g., having one or both of reverse transcriptase activity and endonuclease activity).

In some embodiments, the gene modifying polypeptide comprises an amino acid sequence of SEQ ID NO: 403 or a sequence at least 70%, 75%, 80%, 85%, 90%, 95%, 96%, 97%, 98%, or 99% identical thereto, or a functional fragment thereof (e.g., having one or both of reverse transcriptase activity and endonuclease activity). In some embodiments, the gene modifying polypeptide comprises an amino acid sequence of SEQ ID NO: 403. In some embodiments, the sequence that binds the polypeptide comprises:
a 5′ UTR having a sequence of SEQ ID NO: 703 or a sequence at least 80%, 85%, 90%, 95%, 96%, 97%, 98%, or 99% identical thereto; and
a 3′ UTR having a sequence of SEQ ID NO: 803 or a sequence at least 80%, 85%, 90%, 95%, 96%, 97%, 98%, or 99% identical thereto.

In some embodiments, the gene modifying polypeptide comprises an amino acid sequence of SEQ ID NO: 404 or a sequence at least 70%, 75%, 80%, 85%, 90%, 95%, 96%, 97%, 98%, or 99% identical thereto, or a functional fragment thereof (e.g., having one or both of reverse transcriptase activity and endonuclease activity). In some embodiments, the gene modifying polypeptide comprises an amino acid sequence of SEQ ID NO: 404. In some embodiments, the sequence that binds the polypeptide comprises:
a 5′ UTR having a sequence of SEQ ID NO: 704 or a sequence at least 80%, 85%, 90%, 95%, 96%, 97%, 98%, or 99% identical thereto; and
a 3′ UTR having a sequence of SEQ ID NO: 804 or a sequence at least 80%, 85%, 90%, 95%, 96%, 97%, 98%, or 99% identical thereto.

In some embodiments, the gene modifying polypeptide comprising an amino acid sequence of SEQ ID NO: 405, or a sequence having at least 70%, 75%, 80%, 85%, 90%, 95%, 96%, 97%, 98%, or 99% identity thereto, wherein one, two, or three of:
i) amino acid position 444 is other than P, e.g., is A,
ii) amino acid position 848 is other than D, e.g., is G; and
iii) amino acid position 875 is other than T, e.g., is A
or a functional fragment thereof (e.g., having one or both of reverse transcriptase activity and endonuclease activity).

In some embodiments, the gene modifying polypeptide comprising an amino acid sequence of SEQ ID NO:405, or a sequence having at least 70%, 75%, 80%, 85%, 90%, 95%, 96%, 97%, 98%, or 99% identity thereto, wherein one, two, or three of:
i) amino acid position 444 is A,
ii) amino acid position 848 is G; and
iii) amino acid position 875 is A
or a functional fragment thereof (e.g., having one or both of reverse transcriptase activity and endonuclease activity).

In some embodiments, the gene modifying polypeptide comprises an amino acid sequence of SEQ ID NO: 405 or a sequence at least 70%, 75%, 80%, 85%, 90%, 95%, 96%, 97%, 98%, or 99% identical thereto, or a functional fragment thereof (e.g., having one or both of reverse transcriptase activity and endonuclease activity). In some embodiments, the gene modifying polypeptide comprises an amino acid sequence of SEQ ID NO: 405. In some embodiments, the sequence that binds the polypeptide comprises:
a 5′ UTR having a sequence of SEQ ID NO: 705 or a sequence at least 80%, 85%, 90%, 95%, 96%, 97%, 98%, or 99% identical thereto; and
a 3′ UTR having a sequence of SEQ ID NO: 805 or a sequence at least 80%, 85%, 90%, 95%, 96%, 97%, 98%, or 99% identical thereto.

In some embodiments, the gene modifying polypeptide comprises an amino acid sequence of SEQ ID NO: 406 or a sequence at least 70%, 75%, 80%, 85%, 90%, 95%, 96%, 97%, 98%, or 99% identical thereto, or a functional fragment thereof (e.g., having one or both of reverse transcriptase activity and endonuclease activity). In some embodiments, the gene modifying polypeptide comprises an amino acid sequence of SEQ ID NO: 406. In some embodiments, the sequence that binds the polypeptide comprises:
a 5′ UTR having a sequence of SEQ ID NO: 706 or a sequence at least 80%, 85%, 90%, 95%, 96%, 97%, 98%, or 99% identical thereto; and
a 3′ UTR having a sequence of SEQ ID NO: 806 or a sequence at least 80%, 85%, 90%, 95%, 96%, 97%, 98%, or 99% identical thereto.

In some embodiments, the gene modifying polypeptide comprises an amino acid sequence of SEQ ID NO: 407 or a sequence at least 70%, 75%, 80%, 85%, 90%, 95%, 96%, 97%, 98%, or 99% identical thereto, or a functional fragment thereof (e.g., having one or both of reverse transcriptase activity and endonuclease activity). In some embodiments, the gene modifying polypeptide comprises an amino acid sequence of SEQ ID NO: 407. In some embodiments, the sequence that binds the polypeptide comprises:
a 5′ UTR having a sequence of SEQ ID NO: 707 or a sequence at least 80%, 85%, 90%, 95%, 96%, 97%, 98%, or 99% identical thereto; and
a 3′ UTR having a sequence of SEQ ID NO: 807 or a sequence at least 80%, 85%, 90%, 95%, 96%, 97%, 98%, or 99% identical thereto.

In some embodiments, the gene modifying polypeptide comprises an amino acid sequence of SEQ ID NO: 408 or a sequence at least 70%, 75%, 80%, 85%, 90%, 95%, 96%, 97%, 98%, or 99% identical thereto, or a functional fragment thereof (e.g., having one or both of reverse transcriptase activity and endonuclease activity). In some embodiments, the gene modifying polypeptide comprises an amino acid sequence of SEQ ID NO: 408. In some embodiments, the sequence that binds the polypeptide comprises:
a 5′ UTR having a sequence of SEQ ID NO: 708 or a sequence at least 80%, 85%, 90%, 95%, 96%, 97%, 98%, or 99% identical thereto; and
a 3′ UTR having a sequence of SEQ ID NO: 808 or a sequence at least 80%, 85%, 90%, 95%, 96%, 97%, 98%, or 99% identical thereto.

In some embodiments, the gene modifying polypeptide comprises an amino acid sequence of SEQ ID NO: 409 or a sequence at least 70%, 75%, 80%, 85%, 90%, 95%, 96%, 97%, 98%, or 99% identical thereto, or a functional fragment thereof (e.g., having one or both of reverse transcriptase activity and endonuclease activity). In some embodiments, the gene modifying polypeptide comprises an amino acid sequence of SEQ ID NO: 409. In some embodiments, the sequence that binds the polypeptide comprises:
a 5′ UTR having a sequence of SEQ ID NO: 709 or a sequence at least 80%, 85%, 90%, 95%, 96%, 97%, 98%, or 99% identical thereto; and
a 3′ UTR having a sequence of SEQ ID NO: 809 or a sequence at least 80%, 85%, 90%, 95%, 96%, 97%, 98%, or 99% identical thereto.

In some embodiments, the gene modifying polypeptide comprising an amino acid sequence of SEQ ID NO: 410, or a sequence having at least 70%, 75%, 80%, 85%, 90%, 95%, 96%, 97%, 98%, or 99% identity thereto, wherein one or both of:
i) amino acid position 476 is a non-polar residue, e.g., is L, and
ii) amino acid position 524 is a non-polar residue, e.g., is L;
or a functional fragment thereof (e.g., having one or both of reverse transcriptase activity and endonuclease activity).

In some embodiments, the gene modifying polypeptide comprising an amino acid sequence of SEQ ID NO: 410, or a sequence having at least 70%, 75%, 80%, 85%, 90%, 95%, 96%, 97%, 98%, or 99% identity thereto, wherein one or both of:
i) amino acid position 476 is L, and
ii) amino acid position 524 is L;
or a functional fragment thereof (e.g., having one or both of reverse transcriptase activity and endonuclease activity).

In some embodiments, the gene modifying polypeptide comprises an amino acid sequence of SEQ ID NO: 410 or a sequence at least 70%, 75%, 80%, 85%, 90%, 95%, 96%, 97%, 98%, or 99% identical thereto, or a functional fragment thereof (e.g., having one or both of reverse transcriptase activity and endonuclease activity). In some embodiments, the gene modifying polypeptide comprises an amino acid sequence of SEQ ID NO: 410. In some embodiments, the sequence that binds the polypeptide comprises:
a 5′ UTR having a sequence of SEQ ID NO: 710 or a sequence at least 80%, 85%, 90%, 95%, 96%, 97%, 98%, or 99% identical thereto; and
a 3′ UTR having a sequence of SEQ ID NO: 810 or a sequence at least 80%, 85%, 90%, 95%, 96%, 97%, 98%, or 99% identical thereto.

In some embodiments, the gene modifying polypeptide comprises an amino acid sequence of SEQ ID NO: 420, or a sequence having at least 70%, 75%, 80%, 85%, 90%, 95%, 96%, 97%, 98%, or 99% identity thereto, wherein amino acid position 191 is other than D, e.g., is A, or a fragment thereof having reverse transcriptase activity. In some embodiments, the gene modifying polypeptide comprises an amino acid sequence of SEQ ID NO: 420, or a sequence having no more than 10, 20, 30, 40, 50, 60, 70, 80, 90, or 100 nucleotide differences thereto, wherein amino acid position 191 is other than D, e.g., is A, or a fragment thereof having reverse transcriptase activity. In some embodiments, the gene modifying polypeptide has an endonuclease activity of less than 20%, 15%, 10%, or 5% of that of a polypeptide of SEQ ID NO: 405 in an assay according to Example 4.

In certain embodiments, the gene modifying polypeptide further comprises a heterologous protein domain. In some embodiments, a linker (e.g., as described in Table L1 herein) is disposed between the heterologous protein domain and the amino acid sequence having at least 70%, 75%, 80%, 85%, 90%, 95%, 96%, 97%, 98%, or 99% identity to SEQ ID NO: 405, or fragment thereof.

In some embodiments, the gene modifying polypeptide comprises an amino acid sequence of SEQ ID NO: 421, or a sequence having at least 70%, 75%, 80%, 85%, 90%, 95%, 96%, 97%, 98%, or 99% identity thereto, wherein amino acid position 191 is other than D, e.g., is A, or a fragment thereof having reverse transcriptase activity. In some embodiments, the gene modifying polypeptide comprises an amino acid sequence of SEQ ID NO: 421, or a sequence having no more than 10, 20, 30, 40, 50, 60, 70, 80, 90, or 100 nucleotide differences thereto, wherein amino acid position 191 is other than D, e.g., is A, or a fragment thereof having reverse transcriptase activity. In some embodiments, the gene modifying polypeptide has an endonuclease activity of less than 20%, 15%, 10%, or 5% of that of a polypeptide of SEQ ID NO: 406 in an assay according to Example 4.

In certain embodiments, the gene modifying polypeptide further comprises a heterologous protein domain. In some embodiments, a linker (e.g., as described in Table L1 herein) is disposed between the heterologous protein domain and the amino acid sequence having at least 70%, 75%, 80%, 85%, 90%, 95%, 96%, 97%, 98%, or 99% identity to SEQ ID NO: 406, or fragment thereof.

Table R1 provides gene modifying polypeptides comprising retrotransposon elements, altered for improved efficiency of integration into the human genome. Retrotransposase polypeptides were improved through consensus mapping to re-derive the optimal amino acid sequence. Template molecules for use with cognate retrotransposase enzymes were mapped back to their host genomes and flanking genomic DNA used to elucidate target site motifs. When detectable, conserved sequence motifs from the flanking genomic DNA of endogenous occurrences of an element were aligned to the human genome, and new sequences were derived from the human genome as 5′ or 3′ “Human Homology Arms.” In some embodiments, a template RNA described herein comprises one or both of a first homology domain comprising a sequence of a 5′ Human Homology Arm of Table R1 (or a sequence having at least 80%, 85%, 90%, 95%, 96%, 97%, 98%, or 99% identity thereto) and a second homology domain comprising a sequence of a 3′ Human Homology Arm of Table R1 (or a sequence having at least 80%, 85%, 90%, 95%, 96%, 97%, 98%, or 99% identity thereto).

TABLE R1
Retrotransposase systems with improved integration activity

| | | | | Consensus | | | | |
|---|---|---|---|---|---|---|---|---|
| | | | | Optimized | 5′ Human | 3′ Human | | |
| | | | Target | Protein | Homology | Homology | | |
| Element | Organism | Domains | Motif | Sequence | Arm | Arm | 5′ UTR | 3′ UTR |
| L2-2_DRe | Danio | RT and | TGAGCA | (SEQ ID NO: 400) | (SEQ ID | a | (SEQ | (SEQ ID |
| (1) | rerio | EN | CGGTAG | MCFLIPVVTNTR | NO: 500) | | ID NO: | NO: |
| | | | CATTAG | KTREVRCRRNP | GTTTCGC | | 700) | 800) |
| | | | TCGa | HNLRSIHVSTIS | GTCGTCC | | GCAG | TAATC |
| | | | (SEQ ID | QLSLSVGLWNC | GCCTCA | | GAGA | TGCAA |
| | | | NO: | QSAVNKADFITS | GTTTCGG | | AGCA | TTGCC |
| | | | 15405) | IATYSDYNLMA | CCCTTGT | | CTTTA | TCTCT |
| | | | | LTETWLRPEDT | TTTGACT | | GCAG | GAATA |
| | | | | ATHATLSANFSF | CGGGAG | | CATC | TCACA |
| | | | | SHTPRQTGRGG | GCGTGTC | | TAGA | CTAAC |
| | | | | GTGLLISKEWKF | CAAACG | | ACAG | TGTAC |
| | | | | TLIPSLPTISSFEF | CCAGGT | | CAGC | CCAAA |
| | | | | HAVTIIHPFYIN | AACCAC | | CTGT | AAAA |
| | | | | VVVIYRPPGKL | TATATAG | | AAGT | AAAA |
| | | | | GHFLDELDVLL | TGAGCA | | ACAT | AAAA |
| | | | | SSFSNFATPLLV | CGGTAG | | TTAA | AAAA |
| | | | | LGDFNIYVDKP | CATTAGT | | GATT | AAAA |
| | | | | QAADFQTLLAS | CG | | TGTTT | AAATA |
| | | | | FDLKRAPTSAT | | | CAGT | AAAA |
| | | | | HKSGNQLDLIY | | | TGTT | WACT |
| | | | | TRHCFTDQTIVT | | | GTGT | ACTAA |
| | | | | PLQISDHFLLSL | | | ATGG | TACTT |
| | | | | NIHITPEPPHTPT | | | TTTG | CCCTT |
| | | | | LVTFRRNLRSLS | | | AGGA | CTTAG |
| | | | | PNRLSTIVSDSL | | | CTTGT | ACTTT |
| | | | | PPSRKLTALDSN | | | TTCC | ACAGA |
| | | | | SATNTLCSTLAS | | | AGCT | CCTGA |
| | | | | CLDRLCPLASRP | | | GTTT | AACTT |
| | | | | ARASPPAPWLS | | | GTGT | GCCTA |
| | | | | DALREHRSKLR | | | AAAG | TAGCA |
| | | | | AAERIWRKTKN | | | TTGT | CTTAT |
| | | | | PAHLLTYQTLLS | | | AGGA | TCATT |
| | | | | SFSAEVTSAKQT | | | CATTT | GTTGC |
| | | | | YYRLKINNATN | | | AAAC | TCTTA |
| | | | | PRLLFKTFSSLL | | | TTGCT | GTTGT |
| | | | | YPPPPPASSTLT | | | TTCA | GTAAA |
| | | | | TDDFATFFCTKT | | | GTTG | TTGCT |
| | | | | AKISAQFAAPTT | | | TGTA | TCCTT |
| | | | | NTQDTTPTPHTL | | | TAAT | GTCCT |
| | | | | TSFSQLSESEVS | | | ACTA | CATTT |
| | | | | KLVLSSHATTCP | | | GAAG | GTAAG |
| | | | | LDPIPSHLLQAIS | | | TTGTT | TCGCT |
| | | | | PAVIPTLTHIINT | | | TAGC | TTGGA |
| | | | | SLDSGLFPTTFK | | | CACT | TAAAA |
| | | | | QARVTPLLKKP | | | GTTTC | GCGTC |
| | | | | NLDHTLLENYR | | | CTTG | TGCTA |
| | | | | PVSLLPFMAKIL | | | GTTA | AATGA |
| | | | | EKVVFNQVLDF | | | CTAT | CTAAA |
| | | | | LTQNNLMDNK | | | AAGA | TGTAA |
| | | | | QSGFKKGHSTE | | | GCTT | ATGTA |
| | | | | TALLSVVEDLR | | | GTGT | AATGT |
| | | | | LAKADSKSSVLI | | | AGCG | |
| | | | | LLDLSAAFDTV | | | AACG | |
| | | | | NHQILLSTLESL | | | CAGA | |
| | | | | GVAGTVIQWFR | | | CGCG | |
| | | | | SYLSDRSFRVS | | | GTTC | |
| | | | | WRGEVSNLQHL | | | GCGT | |
| | | | | NTGVPQGSVLG | | | CGTC | |
| | | | | PLLFSIYTSSLGP | | | CGCC | |
| | | | | VIQRHGFSYHC | | | TCAG | |
| | | | | YADDTQLYLSF | | | TTTCG | |
| | | | | HPDDPSVPARIS | | | GCCC | |
| | | | | ACLLDISHWMK | | | TTGTT | |
| | | | | DHHLQLNLAKT | | | TTGA | |
| | | | | EMLVVSANPTL | | | CTCG | |
| | | | | HHNFSIQMDGA | | | GGAG | |
| | | | | TITASKMVKSL | | | GCGT | |
| | | | | GVTIDDQLNFSD | | | GTCC | |
| | | | | HISRTARSCRFA | | | AAAC | |
| | | | | LYNIRKIRPFLSE | | | GCCA | |
| | | | | HAAQLLVQALV | | | GGTA | |
| | | | | LSKLDYCNSLL | | | ACCA | |
| | | | | AGLPANSIKPLQ | | | CTAT | |
| | | | | LLQNAAARVVF | | | ATAG | |
| | | | | NEPKRAHVTPL | | | TGAG | |
| | | | | LVRLHWLPVAA | | | CACG | |
| | | | | RIKFKTLMFAY | | | GTAG | |
| | | | | KVTSGLAPSYL | | | CATT | |
| | | | | HSLLQIYVPSRN | | | AGTC | |
| | | | | LRSVNERRLVV | | | GGCA | |
| | | | | PSQRGKKSLSRT | | | GGAG | |
| | | | | LTLNLPSWWNE | | | AAGC | |
| | | | | LPNCIRTAESLAI | | | ACTTT | |
| | | | | FKKRLKTQLFSL | | | AGCA | |
| | | | | HFTS | | | GCAT | |
| | | | | | | | CTAG | |
| | | | | | | | AACA | |
| | | | | | | | GCAG | |
| | | | | | | | CCTG | |
| | | | | | | | TAAG | |
| | | | | | | | TACA | |
| | | | | | | | TTTA | |
| | | | | | | | AGAT | |
| | | | | | | | TTGTT | |
| | | | | | | | TCAG | |
| | | | | | | | TTGTT | |
| | | | | | | | GTGT | |
| | | | | | | | ATGG | |
| | | | | | | | TTTG | |
| | | | | | | | AGGA | |
| | | | | | | | CTTGT | |
| | | | | | | | TTCC | |
| | | | | | | | AGCT | |
| | | | | | | | GTTT | |
| | | | | | | | GTGT | |
| | | | | | | | AAAG | |
| | | | | | | | TTGT | |
| | | | | | | | AGGA | |
| | | | | | | | CATTT | |
| | | | | | | | AAAC | |
| | | | | | | | TTGCT | |
| | | | | | | | TTCA | |
| | | | | | | | GTTG | |
| | | | | | | | TGTA | |
| | | | | | | | TAAT | |
| | | | | | | | ACTA | |
| | | | | | | | GAAG | |
| | | | | | | | TTGTT | |
| | | | | | | | TAGC | |
| | | | | | | | CACT | |
| | | | | | | | GTTTC | |
| | | | | | | | CTTG | |
| | | | | | | | GTTA | |
| | | | | | | | CTAT | |
| | | | | | | | AAGA | |
| | | | | | | | GCTT | |
| | | | | | | | GTGT | |
| | | | | | | | AGCG | |
| | | | | | | | AACG | |
| | | | | | | | CAGA | |
| | | | | | | | CGCG | |
| | | | | | | | GTTC | |
| | | | | | | | GCGT | |
| | | | | | | | CGTC | |
| | | | | | | | CGCC | |
| | | | | | | | TCAG | |
| | | | | | | | TTTCG | |
| | | | | | | | GCCC | |
| | | | | | | | TTGTT | |
| | | | | | | | TTGA | |
| | | | | | | | CTCG | |
| | | | | | | | GGAG | |
| | | | | | | | GCGT | |
| | | | | | | | GTCC | |
| | | | | | | | AAAC | |
| | | | | | | | GCCA | |
| | | | | | | | GGTA | |
| | | | | | | | ACCA | |
| | | | | | | | CTAT | |
| | | | | | | | ATAG | |
| | | | | | | | TGAG | |
| | | | | | | | CACG | |
| | | | | | | | GTAG | |
| | | | | | | | CATT | |
| | | | | | | | AGTC | |
| | | | | | | | GGCA | |
| | | | | | | | GGAG | |
| | | | | | | | AAGC | |
| | | | | | | | ACTTT | |
| | | | | | | | AGCA | |
| | | | | | | | GCAT | |
| | | | | | | | CTAG | |
| | | | | | | | AACA | |
| | | | | | | | GCAG | |
| | | | | | | | CCTG | |
| | | | | | | | TAAG | |
| | | | | | | | TACA | |
| | | | | | | | TTTA | |
| | | | | | | | AGAT | |
| | | | | | | | TTGTT | |
| | | | | | | | TCAG | |
| | | | | | | | TTGTT | |
| | | | | | | | GTGT | |
| | | | | | | | ATGG | |
| | | | | | | | TTTG | |
| | | | | | | | AGGA | |
| | | | | | | | CTTGT | |
| | | | | | | | TTCC | |
| | | | | | | | AGCT | |
| | | | | | | | GTTT | |
| | | | | | | | GTGT | |
| | | | | | | | AAAG | |
| | | | | | | | TTGT | |
| | | | | | | | AGGA | |
| | | | | | | | CATTT | |
| | | | | | | | AAAC | |
| | | | | | | | TTGCT | |
| | | | | | | | TTCA | |
| | | | | | | | GTTG | |
| | | | | | | | TGTA | |
| | | | | | | | TAAT | |
| | | | | | | | ACTA | |
| | | | | | | | GAAG | |
| | | | | | | | TTGTT | |
| | | | | | | | TAGC | |
| | | | | | | | CACT | |
| | | | | | | | GTTTC | |
| | | | | | | | CTTG | |
| | | | | | | | GTTA | |
| | | | | | | | CTAT | |
| | | | | | | | AAGA | |
| | | | | | | | GCTT | |
| | | | | | | | GTGT | |
| | | | | | | | AGCG | |
| | | | | | | | AACG | |
| | | | | | | | CAGA | |
| | | | | | | | CGCG | |
| | | | | | | | GTTC | |
| | | | | | | | GCGT | |
| | | | | | | | CGTC | |
| | | | | | | | CGCC | |
| | | | | | | | TCAG | |
| | | | | | | | TTTCG | |
| | | | | | | | GCCC | |
| | | | | | | | TTGTT | |
| | | | | | | | TTGA | |
| | | | | | | | CTCTC | |
| | | | | | | | GAGG | |
| | | | | | | | CGTG | |
| | | | | | | | TCCA | |
| | | | | | | | GCTG | |
| | | | | | | | AATT | |
| | | | | | | | CAAT | |
| | | | | | | | CAGC | |
| | | | | | | | TAGT | |
| | | | | | | | GCTTT | |
| | | | | | | | GGGG | |
| | | | | | | | TTAT | |
| | | | | | | | ATAA | |
| | | | | | | | ACAA | |
| | | | | | | | CTAG | |
| | | | | | | | TTCA | |
| | | | | | | | CCGC | |
| | | | | | | | GGCA | |
| | | | | | | | GCGG | |
| | | | | | | | TCGC | |
| | | | | | | | GGCA | |
| | | | | | | | GCCT | |
| | | | | | | | CGTG | |
| | | | | | | | TGAA | |
| | | | | | | | GACC | |
| | | | | | | | GACG | |
| | | | | | | | AGGG | |
| | | | | | | | TAAA | |
| | | | | | | | GACC | |
| | | | | | | | ATCG | |
| | | | | | | | ACTC | |
| | | | | | | | TACC | |
| | | | | | | | TGCG | |
| | | | | | | | CGAC | |
| | | | | | | | TCCA | |
| | | | | | | | CCGA | |
| | | | | | | | GCAA | |
| | | | | | | | AGAC | |
| | | | | | | | ACCG | |
| | | | | | | | ACAA | |
| | | | | | | | AGCA | |
| | | | | | | | CTTG | |
| | | | | | | | AGTA | |
| | | | | | | | CTTTA | |
| | | | | | | | CTGT | |
| | | | | | | | ATTG | |
| | | | | | | | TTTTA | |
| | | | | | | | CTTTA | |
| | | | | | | | CACT | |
| | | | | | | | TATTT | |
| | | | | | | | TTTGT | |
| | | | | | | | TGTC | |
| | | | | | | | AGTG | |
| | | | | | | | CACT | |
| | | | | | | | TTTAT | |
| | | | | | | | T | |
| L2-2_Dre | Danio | RT and | TGAGCA | (SEQ ID NO: 401) | (SEQ ID | a | (SEQ | (SEQ ID |
| (2) | rerio | EN | CGGTAG | MCFLIPVVTNTR | NO: 500) | | ID NO: | NO: |
| | | | CATTAG | KTREVRCRRNP | GTTTCGC | | 701) | 801) |
| | | | TCGa | HNLRSIHVSTIS | GTCGTCC | | GCAG | TAATC |
| | | | (SEQ ID | QLSLSVGLWNC | GCCTCA | | GAGA | TGCAA |
| | | | NO: | QSAVNKADFITS | GTTTCGG | | AGCA | TTGCC |
| | | | 15405) | IATYSDYNLMA | CCCTTGT | | CTTTA | TCTCT |
| | | | | LTETWLRPEDT | TTTGACT | | GCAG | GAATA |
| | | | | ATHATLSANFSF | CGGGAG | | CATC | TCACA |
| | | | | SHTPRQTGRGG | GCGTGTC | | TAGA | CTAAC |
| | | | | GTGLLISKEWKF | CAAACG | | ACAG | TGTAC |
| | | | | TLIPSLPTISSFEF | CCAGGT | | CAGC | CCAAA |
| | | | | HAVTIIHPFYIN | AACCAC | | CTGT | AAAA |
| | | | | VVVIYRPPGKL | TATATAG | | AAGT | AAAA |
| | | | | GHFLDELDVLL | TGAGCA | | ACAT | AAAA |
| | | | | SSFSNFATPLLV | CGGTAG | | TTAA | AAAA |
| | | | | LGDFNIYVDKP | CATTAGT | | GATT | AAAA |
| | | | | QAADFQTLLAS | CG | | TGTTT | AAATA |
| | | | | FDLKRAPTSAT | | | CAGT | AAAA |
| | | | | HKSGNQLDLIY | | | TGTT | WACT |
| | | | | TRHCFTDQTIVT | | | GTGT | ACTAA |
| | | | | PLQISDHFLLSL | | | ATGG | TACTT |
| | | | | NIHITPEPPHTPT | | | TTTG | CCCTT |
| | | | | LVTFRRNLRSLS | | | AGGA | CTTAG |
| | | | | PNRLSTIVSDSL | | | CTTGT | ACTTT |
| | | | | PPSRKLTALDSN | | | TTCC | ACAGA |
| | | | | SATNTLCSTLAS | | | AGCT | CCTGA |
| | | | | CLDRLCPLASRP | | | GTTT | AACTT |
| | | | | ARASPPAPWLS | | | GTGT | GCCTA |
| | | | | DALREHRSKLR | | | AAAG | TAGCA |
| | | | | AAERIWRKTKN | | | TTGT | CTTAT |
| | | | | PAHLLTYQTLLS | | | AGGA | TCATT |
| | | | | SFSAEVTSAKQT | | | CATTT | GTTGC |
| | | | | YYRLKINNATN | | | AAAC | TCTTA |
| | | | | PRLLFKTFSSLL | | | TTGCT | GTTGT |
| | | | | YPPPPPASSTLT | | | TTCA | GTAAA |
| | | | | TDDFATFFCTKT | | | GTTG | TTGCT |
| | | | | AKISAQFAAPTT | | | TGTA | TCCTT |
| | | | | NTQDTTPTPHTL | | | TAAT | GTCCT |
| | | | | TSFSQLSESEVS | | | ACTA | CATTT |
| | | | | KLVLSSHATTCP | | | GAAG | GTAAG |
| | | | | LDPIPSHLLQAIS | | | TTGTT | TCGCT |
| | | | | PAVIPTLTHIINT | | | TAGC | TTGGA |
| | | | | SLDSGLFPTTFK | | | CACT | TAAAA |
| | | | | QARVTPLLKKP | | | GTTTC | GCGTC |
| | | | | NLDHTLLENYR | | | CTTG | TGCTA |
| | | | | PVSLLPFMAKIL | | | GTTA | AATGA |
| | | | | EKVVFNQVLDF | | | CTAT | CTAAA |
| | | | | LTQNNLMDNK | | | AAGA | TGTAA |
| | | | | QSGFKKGHSTE | | | GCTT | ATGTA |
| | | | | TALLSVVEDLR | | | GTGT | AATGT |
| | | | | LAKADSKSSVLI | | | AGCG | |
| | | | | LLDLSAAFDTV | | | AACG | |
| | | | | NHQILLSTLESL | | | CAGA | |
| | | | | GVAGTVIQWFR | | | CGCG | |
| | | | | SYLSDRSFRVS | | | GTTC | |
| | | | | WRGEVSNLQHL | | | GCGT | |
| | | | | NTGVPQGSVLG | | | CGTC | |
| | | | | PLLFSIYTSSLGP | | | CGCC | |
| | | | | VIQRHGFSYHC | | | TCAG | |
| | | | | YADDTQLYLSF | | | TTTCG | |
| | | | | HPDDPSVPARIS | | | GCCC | |
| | | | | ACLLDISHWMK | | | TTGTT | |
| | | | | DHHLQLNLAKT | | | TTGA | |
| | | | | EMLVVSANPTL | | | CTCG | |
| | | | | HHNFSIQMDGA | | | GGAG | |
| | | | | TITASKMVKSL | | | GCGT | |
| | | | | GVTIDDQLNFSD | | | GTCC | |
| | | | | HISRTARSCRFA | | | AAAC | |
| | | | | LYNIRKIRPFLSE | | | GCCA | |
| | | | | HAAQLLVQALV | | | GGTA | |
| | | | | LSKLDYCNSLL | | | ACCA | |
| | | | | AGLPANSIKPLQ | | | CTAT | |
| | | | | LLQNAAARVVF | | | ATAG | |
| | | | | NEPKRAHVTPL | | | TGAG | |
| | | | | LVRLHWLPVAA | | | CACG | |
| | | | | RIKFKALMFAY | | | GTAG | |
| | | | | KVTSGLAPSYLL | | | CATT | |
| | | | | SLLQIYVPSRNL | | | AGTC | |
| | | | | RSVNERRLVVP | | | GGCA | |
| | | | | SQRGKKSLSRTL | | | GGAG | |
| | | | | TLNLPSWWNEL | | | AAGC | |
| | | | | PNCIRTAESLAIF | | | ACTTT | |
| | | | | KKRLKTQLFSL | | | AGCA | |
| | | | | HFTS | | | GCAT | |
| | | | | | | | CTAG | |
| | | | | | | | AACA | |
| | | | | | | | GCAG | |
| | | | | | | | CCTG | |
| | | | | | | | TAAG | |
| | | | | | | | TACA | |
| | | | | | | | TTTA | |
| | | | | | | | AGAT | |
| | | | | | | | TTGTT | |
| | | | | | | | TCAG | |
| | | | | | | | TTGTT | |
| | | | | | | | GTGT | |
| | | | | | | | ATGG | |
| | | | | | | | TTTG | |
| | | | | | | | AGGA | |
| | | | | | | | CTTGT | |
| | | | | | | | TTCC | |
| | | | | | | | AGCT | |
| | | | | | | | GTTT | |
| | | | | | | | GTGT | |
| | | | | | | | AAAG | |
| | | | | | | | TTGT | |
| | | | | | | | AGGA | |
| | | | | | | | CATTT | |
| | | | | | | | AAAC | |
| | | | | | | | TTGCT | |
| | | | | | | | TTCA | |
| | | | | | | | GTTG | |
| | | | | | | | TGTA | |
| | | | | | | | TAAT | |
| | | | | | | | ACTA | |
| | | | | | | | GAAG | |
| | | | | | | | TTGTT | |
| | | | | | | | TAGC | |
| | | | | | | | CACT | |
| | | | | | | | GTTTC | |
| | | | | | | | CTTG | |
| | | | | | | | GTTA | |
| | | | | | | | CTAT | |
| | | | | | | | AAGA | |
| | | | | | | | GCTT | |
| | | | | | | | GTGT | |
| | | | | | | | AGCG | |
| | | | | | | | AACG | |
| | | | | | | | CAGA | |
| | | | | | | | CGCG | |
| | | | | | | | GTTC | |
| | | | | | | | GCGT | |
| | | | | | | | CGTC | |
| | | | | | | | CGCC | |
| | | | | | | | TCAG | |
| | | | | | | | TTTCG | |
| | | | | | | | GCCC | |
| | | | | | | | TTGTT | |
| | | | | | | | TTGA | |
| | | | | | | | CTCG | |
| | | | | | | | GGAG | |
| | | | | | | | GCGT | |
| | | | | | | | GTCC | |
| | | | | | | | AAAC | |
| | | | | | | | GCCA | |
| | | | | | | | GGTA | |
| | | | | | | | ACCA | |
| | | | | | | | CTAT | |
| | | | | | | | ATAG | |
| | | | | | | | TGAG | |
| | | | | | | | CACG | |
| | | | | | | | GTAG | |
| | | | | | | | CATT | |
| | | | | | | | AGTC | |
| | | | | | | | GGCA | |
| | | | | | | | GGAG | |
| | | | | | | | AAGC | |
| | | | | | | | ACTTT | |
| | | | | | | | AGCA | |
| | | | | | | | GCAT | |
| | | | | | | | CTAG | |
| | | | | | | | AACA | |
| | | | | | | | GCAG | |
| | | | | | | | CCTG | |
| | | | | | | | TAAG | |
| | | | | | | | TACA | |
| | | | | | | | TTTA | |
| | | | | | | | AGAT | |
| | | | | | | | TTGTT | |
| | | | | | | | TCAG | |
| | | | | | | | TTGTT | |
| | | | | | | | GTGT | |
| | | | | | | | ATGG | |
| | | | | | | | TTTG | |
| | | | | | | | AGGA | |
| | | | | | | | CTTGT | |
| | | | | | | | TTCC | |
| | | | | | | | AGCT | |
| | | | | | | | GTTT | |
| | | | | | | | GTGT | |
| | | | | | | | AAAG | |
| | | | | | | | TTGT | |
| | | | | | | | AGGA | |
| | | | | | | | CATTT | |
| | | | | | | | AAAC | |
| | | | | | | | TTGCT | |
| | | | | | | | TTCA | |
| | | | | | | | GTTG | |
| | | | | | | | TGTA | |
| | | | | | | | TAAT | |
| | | | | | | | ACTA | |
| | | | | | | | GAAG | |
| | | | | | | | TTGTT | |
| | | | | | | | TAGC | |
| | | | | | | | CACT | |
| | | | | | | | GTTTC | |
| | | | | | | | CTTG | |
| | | | | | | | GTTA | |
| | | | | | | | CTAT | |
| | | | | | | | AAGA | |
| | | | | | | | GCTT | |
| | | | | | | | GTGT | |
| | | | | | | | AGCG | |
| | | | | | | | AACG | |
| | | | | | | | CAGA | |
| | | | | | | | CGCG | |
| | | | | | | | GTTC | |
| | | | | | | | GCGT | |
| | | | | | | | CGTC | |
| | | | | | | | CGCC | |
| | | | | | | | TCAG | |
| | | | | | | | TTTCG | |
| | | | | | | | GCCC | |
| | | | | | | | TTGTT | |
| | | | | | | | TTGA | |
| | | | | | | | CTCTC | |
| | | | | | | | GAGG | |
| | | | | | | | CGTG | |
| | | | | | | | TCCA | |
| | | | | | | | GCTG | |
| | | | | | | | AATT | |
| | | | | | | | CAAT | |
| | | | | | | | CAGC | |
| | | | | | | | TAGT | |
| | | | | | | | GCTTT | |
| | | | | | | | GGGG | |
| | | | | | | | TTAT | |
| | | | | | | | ATAA | |
| | | | | | | | ACAA | |
| | | | | | | | CTAG | |
| | | | | | | | TTCA | |
| | | | | | | | CCGC | |
| | | | | | | | GGCA | |
| | | | | | | | GCGG | |
| | | | | | | | TCGC | |
| | | | | | | | GGCA | |
| | | | | | | | GCCT | |
| | | | | | | | CGTG | |
| | | | | | | | TGAA | |
| | | | | | | | GACC | |
| | | | | | | | GACG | |
| | | | | | | | AGGG | |
| | | | | | | | TAAA | |
| | | | | | | | GACC | |
| | | | | | | | ATCG | |
| | | | | | | | ACTC | |
| | | | | | | | TACC | |
| | | | | | | | TGCG | |
| | | | | | | | CGAC | |
| | | | | | | | TCCA | |
| | | | | | | | CCGA | |
| | | | | | | | GCAA | |
| | | | | | | | AGAC | |
| | | | | | | | ACCG | |
| | | | | | | | ACAA | |
| | | | | | | | AGCA | |
| | | | | | | | CTTG | |
| | | | | | | | AGTA | |
| | | | | | | | CTTTA | |
| | | | | | | | CTGT | |
| | | | | | | | ATTG | |
| | | | | | | | TTTTA | |
| | | | | | | | CTTTA | |
| | | | | | | | CACT | |
| | | | | | | | TATTT | |
| | | | | | | | TTTGT | |
| | | | | | | | TGTC | |
| | | | | | | | AGTG | |
| | | | | | | | CACT | |
| | | | | | | | TTTAT | |
| | | | | | | | T | |
| RTE-1_MD | Mono- | L1-EN, | NA | (SEQ ID NO: 402) | | | (SEQ | (SEQ ID |
| (1) | delphis | RT, ZF | | MDSTAHPNQGR | | | ID NO: | NO: |
| | domestica | | | GLEKVSQTLPA | | | 702) | 802) |
| | | | | LQTPGQHTAAG | | | gggtgta | tgaaact |
| | | | | GSSPLSGRNQR | | | tggggtg | gcacaaa |
| | | | | KNTKKLLLGAW | | | ctcaggg | gacaata |
| | | | | NIRTLLDRENTP | | | aggggta | gtcattc |
| | | | | RPERRTALIGKE | | | gtatctc | tcgatca |
| | | | | LARYNIDIAALS | | | tggtatg | ccgagag |
| | | | | ETRLPEEGSLSE | | | gagggct | actacca |
| | | | | PTTGYTFFWKG | | | tgtcgtg | ct |
| | | | | RASNEDRIHGV | | | ccctcct | |
| | | | | GLAIKTSLLKQL | | | agggcag | |
| | | | | PDLPVGISERLM | | | ctctcca | |
| | | | | KIRLPLSKDRYA | | | gcctctg | |
| | | | | TIISAYAPTLTST | | | accccca | |
| | | | | EETIEQFYSDLS | | | cctgaca | |
| | | | | AVLHSVPTNDK | | | cccagct | |
| | | | | LILLGDFNARVG | | | ctcactt | |
| | | | | QDHERWKGVL | | | gtggctc | |
| | | | | GKHGVGKMNN | | | ccagtag | |
| | | | | NGLLLLSKCSEF | | | ctgctag | |
| | | | | ELTITNTVFRMA | | | catgtgg | |
| | | | | NKYKTTWMHP | | | cagcggc | |
| | | | | RSKQWHLIDYII | | | cacaccc | |
| | | | | VRRRDIQDVKIT | | | cgggca | |
| | | | | RAMRGAECWT | | | acggctt | |
| | | | | DHRLVRATLQM | | | cgacag | |
| | | | | RIAPRHPKRAQT | | | gccggct | |
| | | | | VRAFYNVSRLR | | | aaacctt | |
| | | | | DPSYLQTFQSCL | | | gtgaggg | |
| | | | | DDKLSAKGPLT | | | tagccat | |
| | | | | GSSTEKWNQFR | | | cgggtca | |
| | | | | DAVKETSKAVL | | | tegaccc | |
| | | | | GPKQRNHQDW | | | ctggtga | |
| | | | | FDENNTAIEDLL | | | accagg | |
| | | | | SKKNKAFMEW | | | gctttgc | |
| | | | | QNNPNSAPKKD | | | tcaccca | |
| | | | | RFKSLQATAQR | | | gcatgtg | |
| | | | | EIRKMQDRWW | | | aagactg | |
| | | | | EKKAEEIQRFAD | | | cttcggc | |
| | | | | MKNYKQFFSAL | | | tgaacag | |
| | | | | KTVYGPLKPTT | | | acggaag | |
| | | | | TPLLSSDGDTLI | | | aaaccaa | |
| | | | | KDKKGISNRWK | | | taagaag | |
| | | | | EHFSQLLNRPSS | | | gttcaac | |
| | | | | VDQSALDQIPQ | | | ggctgag | |
| | | | | NRTIEQLDVPPSI | | | agggcg | |
| | | | | EEVQKAIKQMS | | | acgcagc | |
| | | | | AGKAPGKDGIP | | | aaagcac | |
| | | | | TEVYKALNGKA | | | tgtggag | |
| | | | | LQAFHIVLTSIW | | | tgcttag | |
| | | | | EEEDMPPELRD | | | ggcgtgt | |
| | | | | ASIVALYKNKG | | | tggagca | |
| | | | | SRAACDNYRGI | | | caaagga | |
| | | | | SLLSTAGKILAR | | | caacacg | |
| | | | | VILNRLLSSVSE | | | gccatcc | |
| | | | | QNLPESQCGFRP | | | aatgcag | |
| | | | | DRSTIDMVFTV | | | ctgagga | |
| | | | | RQMQEKCLEQN | | | agtctcc | |
| | | | | LSLYIVFIDLTK | | | agatgta | |
| | | | | AFDTVNRDALW | | | acaattt | |
| | | | | VILSKLGCPAKF | | | ttcgtgc | |
| | | | | VKLIQLFHVDM | | | cactgga | |
| | | | | TGEVLSGGETS | | | cccaggc | |
| | | | | DRFNISNGVKQ | | | ttccaac | |
| | | | | GCVLAPVLFNL | | | gccgaga | |
| | | | | FFTQVLRHAVM | | | gagtggg | |
| | | | | DLDLGVYIKYR | | | actgtct | |
| | | | | LDGSLFDLRRLT | | | ctgtgca | |
| | | | | AKTKTTERLILE | | | tcggctt | |
| | | | | ALFADDCALMA | | | ttccact | |
| | | | | HQENHLQTIVD | | | taaatct | |
| | | | | RFSTATKLFGLT | | | ctttcac | |
| | | | | ISLSKTEVLFQP | | | gcacaag | |
| | | | | APGRPTNQPCIT | | | tatcttt | |
| | | | | IDGTQLSNVNTF | | | gtgcaca | |
| | | | | KYLGSTIANDGS | | | ctcatct | |
| | | | | LDHEINARIQKA | | | atcctaa | |
| | | | | SQALGRLRCKV | | | ccccgtc | |
| | | | | LQHRGVSTATK | | | caccctc | |
| | | | | LKVYNAVVLSS | | | ttcaaga | |
| | | | | LLYGCETWTLY | | | cctgcgg | |
| | | | | RKHMKQLEQFH | | | cgatggg | |
| | | | | QRSLRSIMRIRW | | | ggagtgg | |
| | | | | QDRITNQEVLD | | | cgacgca | |
| | | | | RANSTSIEVMVL | | | acaggtg | |
| | | | | KTQLRWSGHVI | | | gaggtga | |
| | | | | RMDPQRIPRQV | | | ccactgg | |
| | | | | FYGELSAGLRK | | | cagttgt | |
| | | | | QGRPKKRFKDQ | | | agtcacg | |
| | | | | LKSNLKWAGIT | | | atcctgc | |
| | | | | PKQLELAASDR | | | acgtagg | |
| | | | | SSWRTHINHAA | | | cggccca | |
| | | | | TTFEDERRRRLA | | | cggacca | |
| | | | | AARERRHQATT | | | gtggtcg | |
| | | | | APPVTTGVPCP | | | ctcggcc | |
| | | | | MCHKLCASAFG | | | ctgtggg | |
| | | | | LQSHMRVHRR | | | cagcagg | |
| | | | | | | | gacgttc | |
| | | | | | | | ggcagca | |
| | | | | | | | tcctggg | |
| | | | | | | | cgactga | |
| | | | | | | | gcagccc | |
| | | | | | | | tctctag | |
| RTE-1_MD | Mono- | L1-EN, | NA | (SEQ ID NO: 403) | | | (SEQ | (SEQ ID |
| (2) | delphis | RT, ZF | | MDSTAHPNQGR | | | ID NO: | NO: |
| | domestica | | | GLEKVSQTLPA | | | 703) | 803) |
| | | | | LQTPGQHTAAG | | | gggtgta | tgaaact |
| | | | | GSSPLSGRNQR | | | tggggtg | gcacaaa |
| | | | | KNTKKLLLGAW | | | ctcaggg | gacaata |
| | | | | NIRTLLDRENTP | | | aggggta | gtcattc |
| | | | | RPERRTALIGKE | | | gtatctc | tcgatca |
| | | | | LARYNIDIAALS | | | tggtatg | ccgagag |
| | | | | ETRLPEEGSLSE | | | gagggct | actacca |
| | | | | PTTGYTFFWKG | | | tgtcgtg | ct |
| | | | | RASNEDRIHGV | | | ccctcct | |
| | | | | GLAIKTSLLKQL | | | agggcag | |
| | | | | PDLPVGISERLM | | | ctctcca | |
| | | | | KIRLPLSKDRYA | | | gcctctg | |
| | | | | TIISAYAPTLTST | | | accccca | |
| | | | | EETIEQFYSDLS | | | cctgaca | |
| | | | | AVLHSVPTNDK | | | cccagct | |
| | | | | LILLGDFNARVG | | | ctcactt | |
| | | | | QDHERWKGVL | | | gtggctc | |
| | | | | GKHGVGKMNN | | | ccagtag | |
| | | | | NGLLLLSKCSEF | | | ctgctag | |
| | | | | ELTITNTVFRMA | | | catgtgg | |
| | | | | NKYKTTWMHP | | | cagcggc | |
| | | | | RSKQWHLIDYII | | | cacaccc | |
| | | | | VRRRDIQDVKIT | | | cgggca | |
| | | | | RAMRGAECWT | | | acggctt | |
| | | | | DHRLVRATLQM | | | cgacag | |
| | | | | RIAPRHPKRAQT | | | gccggct | |
| | | | | VRAFYNVSRLR | | | aaacctt | |
| | | | | DPSYLQTFQSCL | | | gtgaggg | |
| | | | | DNKLSAKGPLT | | | tagccat | |
| | | | | GSSTEKWNQFR | | | cgggtca | |
| | | | | DAVKETSKAVL | | | tcgaccc | |
| | | | | GPKQRNHQDW | | | ctggtga | |
| | | | | FDENNTAIEDLL | | | accagg | |
| | | | | SKKNKAFMEW | | | gctttgc | |
| | | | | QNNPNSAPKKD | | | tcaccca | |
| | | | | RFKSLQATAQR | | | gcatgtg | |
| | | | | EIRKMQDRWW | | | aagactg | |
| | | | | EKKAEEIQRFAD | | | cttcggc | |
| | | | | MKNYKQFFSAL | | | tgaacag | |
| | | | | KTVYGPLKPTT | | | acggaag | |
| | | | | TPLLSSDGDTLI | | | aaaccaa | |
| | | | | KDKKGISNRWK | | | taagaag | |
| | | | | EHFSQLLNRPSS | | | gttcaac | |
| | | | | VDQSALDQIPQ | | | ggctgag | |
| | | | | NRSIEQLDVPPSI | | | agggcg | |
| | | | | EEVQKAIKQMS | | | acgcagc | |
| | | | | AGKAPGKDGIP | | | aaagcac | |
| | | | | TEVYKALNGKA | | | tgtggag | |
| | | | | LQAFHIVLTSIW | | | tgcttag | |
| | | | | EEEDMPPELRD | | | ggcgtgt | |
| | | | | ASIVALYKNKG | | | tggagca | |
| | | | | SRAACDNYRGI | | | caaagga | |
| | | | | SLLSTAGKILAR | | | caacacg | |
| | | | | VILNRLLSSVSE | | | gccatcc | |
| | | | | QNLPESQCGFRP | | | aatgcag | |
| | | | | DRSTIDMVFTV | | | ctgagga | |
| | | | | RQMQEKCLEQN | | | agtctcc | |
| | | | | LSLYIVFIDLTK | | | agatgta | |
| | | | | AFDTVNRDALW | | | acaattt | |
| | | | | VILSKLGCPAKF | | | ttcgtgc | |
| | | | | VKLIQLFHVDM | | | cactgga | |
| | | | | TGEVLSGGETS | | | cccaggc | |
| | | | | DRFNISNGVKQ | | | ttccaac | |
| | | | | GCVLAPVLFNL | | | gccgaga | |
| | | | | FFTQVLRHAVM | | | gagtggg | |
| | | | | DLDLGVYIKYR | | | actgtct | |
| | | | | LDGSLFDLRRLT | | | ctgtgca | |
| | | | | AKTKTTERLILE | | | tcggctt | |
| | | | | ALFADDCALMA | | | ttccact | |
| | | | | HQENHLQTIVD | | | taaatct | |
| | | | | RFSTATKLFGLT | | | ctttca | |
| | | | | ISLSKTEVLFQP | | | cgcacaa | |
| | | | | APGRPTNQPCIT | | | gtatctt | |
| | | | | IDGTQLSNVNTF | | | tgtgcac | |
| | | | | KYLGSTIANDGS | | | actcatc | |
| | | | | LDHEINARIQKA | | | tatccta | |
| | | | | SQALGRLRCKV | | | accccgt | |
| | | | | LQHRGVSTATK | | | ccaccct | |
| | | | | LKVYNAVVLSS | | | cttcaag | |
| | | | | LLYGCETWTLY | | | acctgcg | |
| | | | | RKHMKQLEQFH | | | gcgatgg | |
| | | | | QRSLRSIMRIRW | | | gggagtg | |
| | | | | QDRITNQEVLD | | | gcgacgc | |
| | | | | RANSTSIEVMVL | | | aacaggt | |
| | | | | KTQLRWSGHVI | | | ggaggtg | |
| | | | | RMDPQRIPRQV | | | accactg | |
| | | | | FYGELSAGLRK | | | gcagttg | |
| | | | | QGRPKKRFKDQ | | | tagtcac | |
| | | | | LKSNLKWAGIT | | | gatcc | |
| | | | | PKQLELAASDR | | | tgcacg | |
| | | | | SSWRTHINHAA | | | taggcgg | |
| | | | | TTFEDERRRRLA | | | cccacgg | |
| | | | | AARERRHQATT | | | accagtg | |
| | | | | APPVTTGVPCP | | | gtcgctc | |
| | | | | MCHKLCASAFG | | | ggccctg | |
| | | | | LQSHMRVHRR | | | tgggcag | |
| | | | | | | | caggga | |
| | | | | | | | cgttcgg | |
| | | | | | | | cagcatc | |
| | | | | | | | ctgggcg | |
| | | | | | | | actgagc | |
| | | | | | | | agccctc | |
| | | | | | | | tctag | |
| Vingi- | Anolis | DNAse- | NA | (SEQ ID NO: 404) | | | (SEQ | (SEQ ID |
| 1_Acar | carol- | like, RT | | MDEYQRSLSRP | | | ID NO: | NO: |
| (1) | inensis | | | LLTIMSINIEGLS | | | 704) | 804) |
| | | | | LAKEELLAKMS | | | GGGG | TAGTT |
| | | | | EDISCDILCIQET | | | GACA | GCTTG |
| | | | | HRDITMRRPKIL | | | CGGA | TGATT |
| | | | | GMQLAVERPHR | | | AAGA | TCTTT |
| | | | | QYGSAIFVRSGV | | | GCCT | TCTTT |
| | | | | AISATSLTEVNN | | | CCCC | TTTAT |
| | | | | IEILSVELDSCTV | | | GAAG | TTTAT |
| | | | | SSLYKPPGADFY | | | ATTG | TTCCA |
| | | | | FTPPTSCHNHEA | | | AGTG | TTATT |
| | | | | HFVVGDFNSHS | | | AATT | TGAAA |
| | | | | CVWGYDEDDR | | | CAGT | TGTAT |
| | | | | NGEAVLTWAD | | | CGGG | TTGCT |
| | | | | NSRMSLLHDSK | | | CGTC | GTACC |
| | | | | LPPSFNSGRWK | | | CCCT | AATGC |
| | | | | RGYNPDLIFVKE | | | GGGC | TTTTG |
| | | | | SISHQCTKRVLN | | | AACG | ACACG |
| | | | | PIPNTQHRPICC | | | TTTCT | AAATA |
| | | | | VAYAAVRPKSV | | | TGTA | AATAA |
| | | | | PFRRRYNFNKA | | | AGCG | A |
| | | | | NWTKFTETLEA | | | GCCG | |
| | | | | AISDIEPSIENYD | | | ATCTT | |
| | | | | LFVEAVKRSSRL | | | TCCA | |
| | | | | SIPRGCRTSYLP | | | CCCC | |
| | | | | GLNEESLNQLQ | | | AAAA | |
| | | | | EYLRLFQENPYS | | | GCAT | |
| | | | | DGTIAAGQKLS | | | TGGA | |
| | | | | TALANAKKDR | | | TGA | |
| | | | | WIELLENLDMS | | | | |
| | | | | KSSRKAWQLLR | | | | |
| | | | | RLDSDPLVNPG | | | | |
| | | | | HANVTPDQIAH | | | | |
| | | | | QLIQNGKTNCS | | | | |
| | | | | RIKMKINRVPEL | | | | |
| | | | | ETHQLSSPLNLK | | | | |
| | | | | ELREAIKRCKTG | | | | |
| | | | | KAPGLDDLMM | | | | |
| | | | | EQIKHLGPKAE | | | | |
| | | | | NWLLKFYNQCL | | | | |
| | | | | AHKQIPRAWRK | | | | |
| | | | | TKIIAILKPGKD | | | | |
| | | | | ASNARNYRPISL | | | | |
| | | | | LCHLYKVYERM | | | | |
| | | | | LLNRLGPVIEPK | | | | |
| | | | | LIAQQAGFRPG | | | | |
| | | | | KNCTGQILHLTE | | | | |
| | | | | HIEEGYEKGCIT | | | | |
| | | | | GTVFVDLTAAY | | | | |
| | | | | DTVQHRKMLH | | | | |
| | | | | KVYHITRDFDFT | | | | |
| | | | | KTVQTLLENRSF | | | | |
| | | | | YVEFQGQKSRW | | | | |
| | | | | RRQKNGLPQGS | | | | |
| | | | | VLAPTLFNIFTN | | | | |
| | | | | DQPQPPLTKSFI | | | | |
| | | | | YADDLGLTTQA | | | | |
| | | | | KDFETVEKQLT | | | | |
| | | | | NALKDLSSYYK | | | | |
| | | | | ENHLKPNPAKT | | | | |
| | | | | QVCAFHLRNRE | | | | |
| | | | | ANRKLKVTWE | | | | |
| | | | | GQELEHCFHPK | | | | |
| | | | | YLGVTLDRTLT | | | | |
| | | | | YRKHCMNTKH | | | | |
| | | | | KVAARNNILRK | | | | |
| | | | | LTGSAWGADPQ | | | | |
| | | | | VIRTSALALSFS | | | | |
| | | | | TAEYACPVWHK | | | | |
| | | | | SAHAKQVDIAL | | | | |
| | | | | NETCRIITGCLK | | | | |
| | | | | PTPVDKLYKLA | | | | |
| | | | | GIAPPDVRREVA | | | | |
| | | | | ANGERKKVEHC | | | | |
| | | | | ESHPLHDYHPPP | | | | |
| | | | | TRLKSRKGFMR | | | | |
| | | | | TTTPLDVPPATA | | | | |
| | | | | RVSLWAAKPGN | | | | |
| | | | | SNWMAPQEGLP | | | | |
| | | | | PGANQEWATW | | | | |
| | | | | KSLNRLRSGVG | | | | |
| | | | | RSKDNLARWH | | | | |
| | | | | YLEESSTLCDCG | | | | |
| | | | | AEQTTQHMYAC | | | | |
| | | | | PQCPASCTEEEL | | | | |
| | | | | FKATDNAVAVA | | | | |
| | | | | RFWSKTI | | | | |
| Vingi- | Anolis | DNAse- | NA | (SEQ ID NO: 405) | | | (SEQ | (SEQ ID |
| 1_Acar | carol- | like, RT | | MDEYQRSLSRP | | | ID NO: | NO: |
| (2) | inensis | | | LLTIMSINIEGLS | | | 705) | 805) |
| | | | | LAKEELLAKMS | | | GGGG | TAGTT |
| | | | | EDISCDILCIQET | | | GACA | GCTTG |
| | | | | HRDITMRRPKIL | | | CGGA | TGATT |
| | | | | GMQLAVERPHR | | | AAGA | TCTTT |
| | | | | QYGSAIFVRSGV | | | GCCT | TCTTT |
| | | | | AISATSLTEVNN | | | CCCC | TTTAT |
| | | | | IEILSVELDSCTV | | | GAAG | TTTAT |
| | | | | SSLYKPPGADFY | | | ATTG | TTCCA |
| | | | | FTPPTSCHNHEA | | | AGTG | TTATT |
| | | | | HFVVGDFNSHS | | | AATT | TGAAA |
| | | | | CVWGYDEDDR | | | CAGT | TGTAT |
| | | | | NGEAVLTWAD | | | CGGG | TTGCT |
| | | | | NSRMSLLHDSK | | | CGTC | GTACC |
| | | | | LPPSFNSGRWK | | | CCCT | AATGC |
| | | | | RGYNPDLIFVKE | | | GGGC | TTTTG |
| | | | | SISHQCTKRVLN | | | AACG | ACACG |
| | | | | PIPNTQHRPICC | | | TTTCT | AAATA |
| | | | | VAYAAVRPKSV | | | TGTA | AATAA |
| | | | | PFRRRYNFNKA | | | AGCG | A |
| | | | | NWTKFTETLEA | | | GCCG | |
| | | | | AISDIEPSIENYD | | | ATCTT | |
| | | | | LFVEAVKRSSRL | | | TCCA | |
| | | | | SIPRGCRTSYLP | | | CCCC | |
| | | | | GLNEESLNQLQ | | | AAAA | |
| | | | | EYLRLFQENPYS | | | GCAT | |
| | | | | DGTIAAGQKLS | | | TGGA | |
| | | | | TALANAKKDR | | | TGA | |
| | | | | WIELLENLDMS | | | | |
| | | | | KSSRKAWQLLR | | | | |
| | | | | RLDSDPLVNPG | | | | |
| | | | | HANVTPDQIAH | | | | |
| | | | | QLIQNGKTNCS | | | | |
| | | | | RIKMKINRVPEL | | | | |
| | | | | ETHQLSSPLNLK | | | | |
| | | | | ELREAIKRCKTG | | | | |
| | | | | KAPGLDDLMM | | | | |
| | | | | EQIKHLGAKAE | | | | |
| | | | | NWLLKFYNQCL | | | | |
| | | | | AHKQIPRAWRK | | | | |
| | | | | TKIIAILKPGKD | | | | |
| | | | | ASNARNYRPISL | | | | |
| | | | | LCHLYKVYERM | | | | |
| | | | | LLNRLGPVIEPK | | | | |
| | | | | LIAQQAGFRPG | | | | |
| | | | | KNCTGQILHLTE | | | | |
| | | | | HIEEGYEKGCIT | | | | |
| | | | | GTVFVDLTAAY | | | | |
| | | | | DTVQHRKMLH | | | | |
| | | | | KVYHITRDFDFT | | | | |
| | | | | KTVQTLLENRSF | | | | |
| | | | | YVEFQGQKSRW | | | | |
| | | | | RRQKNGLPQGS | | | | |
| | | | | VLAPTLFNIFTN | | | | |
| | | | | DQPQPPLTKSFI | | | | |
| | | | | YADDLGLTTQA | | | | |
| | | | | KDFETVEKQLT | | | | |
| | | | | NALKDLSSYYK | | | | |
| | | | | ENHLKPNPAKT | | | | |
| | | | | QVCAFHLRNRE | | | | |
| | | | | ANRKLKVTWE | | | | |
| | | | | GQELEHCFHPK | | | | |
| | | | | YLGVTLDRTLT | | | | |
| | | | | YRKHCMNTKH | | | | |
| | | | | KVAARNNILRK | | | | |
| | | | | LTGSAWGADPQ | | | | |
| | | | | VIRTSALALSFS | | | | |
| | | | | TAEYACPVWHK | | | | |
| | | | | SAHAKQVDIAL | | | | |
| | | | | NETCRIITGCLK | | | | |
| | | | | PTPVDKLYKLA | | | | |
| | | | | GIAPPDVRREVA | | | | |
| | | | | ANGERKKVEHC | | | | |
| | | | | ESHPLHGYHPPP | | | | |
| | | | | TRLKSRKGFMR | | | | |
| | | | | TTTPLDVPPAAA | | | | |
| | | | | RVSLWAAKPGN | | | | |
| | | | | SNWMAPQEGLP | | | | |
| | | | | PGANQEWATW | | | | |
| | | | | KSLNRLRSGVG | | | | |
| | | | | RSKDNLARWH | | | | |
| | | | | YLEESSTLCDCG | | | | |
| | | | | AEQTTQHMYAC | | | | |
| | | | | PQCPASCTEEEL | | | | |
| | | | | FKATDNAVAVA | | | | |
| | | | | RFWSKTI | | | | |
| CR1-1_PH | Parhyale | DNAse I- | NA | (SEQ ID NO: 406) | | | (SEQ | (SEQ ID |
| | hawaiensis | like, RT | | MLYTIFLVYGIL | | | ID NO: | NO: |
| | | | | CFFFVYFCIYLYI | | | 706) | 806) |
| | | | | TVYLCFLFFLFL | | | gcgtggc | tagcagc |
| | | | | LLCGDVESNPG | | | ctgcgcg | gttttgg |
| | | | | PGRARGCRLLY | | | ttatcag | gcttccc |
| | | | | CNIRGLHANLA | | | gccgccc | ggtgttg |
| | | | | ELDFVSRGVDV | | | ccattgt | caagatg |
| | | | | VCCSETLVAGR | | | ccggcca | cttcatt |
| | | | | RHDAELALAGF | | | ccggacg | tgggttg |
| | | | | QSPFRRLCGSGP | | | cctcttt | tattacc |
| | | | | GFRGMAVYVRS | | | gtttgta | tgtccgt |
| | | | | GFCAYRQSVHE | | | tgagctg | gggagct |
| | | | | CACHEIIVVRVC | | | gctttgg | caagcgt |
| | | | | GRLNNYYLFSL | | | gtttggg | gaagttt |
| | | | | YRSPATDDSLY | | | gctctgg | taataat |
| | | | | DCLLTAMASIQS | | | gtagccc | aataata |
| | | | | TDPKAAFVFVG | | | ctgagtt | a |
| | | | | DVNAHHRDWL | | | ggaggaa | |
| | | | | GSASPTDCHGV | | | atgccac | |
| | | | | AALDFCTLSSCV | | | tctaaa | |
| | | | | QLVRGSTHIAG | | | ggcggat | |
| | | | | NCLDLVMTDVP | | | gagacct | |
| | | | | DLMTVTVGSPI | | | cttttgg | |
| | | | | GSSDHSHLVVS | | | tccaacg | |
| | | | | LDLNQVVPVVD | | | gcatccc | |
| | | | | TRRVVFVKSRA | | | tgagagg | |
| | | | | NWVAITRAVRT | | | agatgaa | |
| | | | | LPWRQIIHSEDP | | | caggctg | |
| | | | | VSELNDLTVSIL | | | gtgccat | |
| | | | | ERFVPKRTILVR | | | agctggg | |
| | | | | SRDKPWFDDQC | | | tactcag | |
| | | | | RLAFEAKQAAY | | | tcttccg | |
| | | | | RAWRRSRDRTL | | | cagtctg | |
| | | | | WQTYVDRRAE | | | ctcctct | |
| | | | | AKRVYEEAQRR | | | ctctctt | |
| | | | | LRQRSRESLLSI | | | ccatcag | |
| | | | | DHPHRWWSEL | | | gtagtac | |
| | | | | KGSVFGAEPSLP | | | cacctga | |
| | | | | PLVGPGGGIITD | | | tgctctt | |
| | | | | PLARAELLSAHF | | | ttccttt | |
| | | | | DGKQSRDVIAL | | | cctttct | |
| | | | | PHGCHPEPRLTS | | | cttcttc | |
| | | | | LAFRSGAIKVLL | | | ttcttt | |
| | | | | EGLDPYGGVDP | | | tcggcc | |
| | | | | VGMFPLFYKQL | | | aaagcct | |
| | | | | ADVLAPKLAVIF | | | gtactgg | |
| | | | | RRLIRLGNFPRC | | | tgatggc | |
| | | | | WRTGNITPIPKG | | | agggtct | |
| | | | | PVSPYVANYRPI | | | ggggcgc | |
| | | | | TLTPILSKVFEK | | | gtagata | |
| | | | | LIAGKLGRFAEV | | | gctggcc | |
| | | | | TGLLPAGQFAY | | | gctttcg | |
| | | | | RIGLGCCDALLS | | | tagctat | |
| | | | | VSHHLQSALDC | | | cgttact | |
| | | | | RSEARLVQLDFS | | | gtcgatt | |
| | | | | AAFDRVNHRGL | | | tgttttt | |
| | | | | LYKLESFGVGG | | | gctttatt | |
| | | | | RVLSIIRDFVSER | | | | |
| | | | | TQSVSVDGVLS | | | | |
| | | | | ASVGVVSGVPQ | | | | |
| | | | | GSVLGPLLFVLY | | | | |
| | | | | TSDMFSSLENTL | | | | |
| | | | | INYADDSTLMA | | | | |
| | | | | VIPAPRLRDAVA | | | | |
| | | | | QSLNRDLSRISA | | | | |
| | | | | WCSAWSMKLN | | | | |
| | | | | ASKTKSMIISRS | | | | |
| | | | | RTLVPQHPQLEI | | | | |
| | | | | DDTLLQESSSLE | | | | |
| | | | | ILGVVFDEKLTF | | | | |
| | | | | EPHIRRLVSRAS | | | | |
| | | | | TKIGLLRKVNSV | | | | |
| | | | | FGDSQVARRCF | | | | |
| | | | | YAFLLPVLEYCS | | | | |
| | | | | PVWASAADTHL | | | | |
| | | | | RLLDRLVSSASR | | | | |
| | | | | LCADNDLVNLS | | | | |
| | | | | HRRRVAELCMF | | | | |
| | | | | YKVYNNERHW | | | | |
| | | | | LYSSLPALKVFG | | | | |
| | | | | RETRAACGAHS | | | | |
| | | | | FTLEAVRCRTN | | | | |
| | | | | QFCRCFVPWSA | | | | |
| | | | | KVWNLLPASAF | | | | |
| | | | | GRIGLQAFKSA | | | | |
| | | | | VNGFLLDFL | | | | |
| Crack- | Branch- | RT and | NNNNNN | (SEQ ID NO: 407) | | | (SEQ | (SEQ ID |
| 28_BF | iostoma | EN | NNNNNN | MWEKATSNVH | | | ID NO: | NO: |
| | floridae | | NNNNNN | LQSGTWITQNA | | | 707) | 807) |
| | | | NTNNNN | YSVKINPGLSGV | | | AGCC | AACTT |
| | | | NCNNGT | RLIGRSTTCTER | | | CTAG | GATCT |
| | | | NNNNAN | TERTLNLLVCA | | | TCCCT | GGCTG |
| | | | NNNNNN | TLLLAGDVSPNP | | | | ATGCT |
| | | | NNNNNN | GPDTGGLPVWR | | | | CCGTA |
| | | | NCNNNN | KGIVYAFYNVV | | | | GTCTG |
| | | | NNNNNN | SLPRHLDEIQQL | | | | GACTT |
| | | | NNNNNN | LLRNTRIHVLGL | | | | TGATA |
| | | | TNNNNN | NETRLSDSIPDS | | | | TTGGA |
| | | | NNNNNN | SVDINGYTLYRT | | | | TAATT |
| | | | NNNNNN | DRDRQGGGVG | | | | TTATT |
| | | | NNNTNN | VYVKQTIASQR | | | | ATGTA |
| | | | NNNNNN | RCELEQEDLEV | | | | TTGTA |
| | | | NNNNnnt | CCVEIKPEKARK | | | | TATGT |
| | | | nnnnnnnn | TLLTCVYRPPTS | | | | GCTAT |
| | | | nnnnnnnn | GPDWRNSAESL | | | | GTGTA |
| | | | nnnncnnn | VHKLNQTAEKE | | | | CTTTG |
| | | | nnnnnnnn | NADVAIMGDEN | | | | TACTT |
| | | | nnnnnnng | SDLLTSTQAMSS | | | | TTATG |
| | | | cnnnnnnn | VEFLMGLYQLV | | | | TCCAG |
| | | | ntnnnnnn | PVIREPTRITEKT | | | | GATTA |
| | | | nnnnnnnn | ESCIDNIFVSNP | | | | CCTGA |
| | | | nnnnnnnn | DRYKSSASVAW | | | | AAAGC |
| | | | nnntnnnn | GPSDHNLILTCA | | | | AGGCC |
| | | | nnnnnnna | KAGSEAGAAHR | | | | GATTT |
| | | | nnnnnnnn | CEYRSYKLYTQ | | | | CCGGC |
| | | | n | QSFIDSLKSVRW | | | | CTGAG |
| | | | (SEQ ID | DTVFDCTDVSE | | | | ATGTA |
| | | | NO: | AWNAFKDIFLN | | | | ATTAC |
| | | | 15406) | VADEHAPLRTK | | | | TGGTA |
| | | | | TARENNRPAPW | | | | AAATA |
| | | | | MTDTVKNMMG | | | | AATAA |
| | | | | RRDAARRKAIR | | | | AGTGA |
| | | | | TKDVQDWDTY | | | | AGTGA |
| | | | | RSLRNQTTSIIR | | | | A |
| | | | | KEKKSHFATAV | | | | |
| | | | | SEAKGDQSLM | | | | |
| | | | | WKIINSFTGKSK | | | | |
| | | | | STKRVQKLLRA | | | | |
| | | | | DNTSMSDPGEM | | | | |
| | | | | AQEFNDYFTSC | | | | |
| | | | | ASRLTDGMPDS | | | | |
| | | | | EEDPLRHIPDST | | | | |
| | | | | TKFSFDCVEETE | | | | |
| | | | | VLNELQKLKTK | | | | |
| | | | | KATGLDKIPAK | | | | |
| | | | | LLKDSAPVVAK | | | | |
| | | | | PLAHIFNLSLAS | | | | |
| | | | | GEVPSDWKEAQ | | | | |
| | | | | ITPVHKSGSCAD | | | | |
| | | | | VGNYRPVSVLS | | | | |
| | | | | VTSKVMEKLVC | | | | |
| | | | | NQVTRYLTRCK | | | | |
| | | | | LLTTHQSGFRR | | | | |
| | | | | HHSTATAVQKV | | | | |
| | | | | VEDITSGYNCSK | | | | |
| | | | | VTVALFLDLRK | | | | |
| | | | | AFDSVNHEIMLS | | | | |
| | | | | KLKKFGFDSDA | | | | |
| | | | | MKWFTSYLSER | | | | |
| | | | | LQCTCLQGQYS | | | | |
| | | | | SKTRVSCGVPQ | | | | |
| | | | | GSVLGPLLFCLY | | | | |
| | | | | VNDLPNVIQKC | | | | |
| | | | | SIHMYADDTVL | | | | |
| | | | | YYSAVSVKVCE | | | | |
| | | | | ETVSMDMKRV | | | | |
| | | | | VKWLSENRLLL | | | | |
| | | | | HPDKTKSMLFG | | | | |
| | | | | LPQKLKHAGTT | | | | |
| | | | | VNITDGVNVYE | | | | |
| | | | | QVDSFTYLGITL | | | | |
| | | | | DPALRWAAHV | | | | |
| | | | | QKITKKLLSGLG | | | | |
| | | | | AMGRARAFVTN | | | | |
| | | | | EVLKTMYQTLL | | | | |
| | | | | LAHLEYCATAW | | | | |
| | | | | LPSLAQGNKTL | | | | |
| | | | | MLQLDRLVNRA | | | | |
| | | | | ARLITGHKLRD | | | | |
| | | | | HVTVDNLRAEA | | | | |
| | | | | GIDSVRKRTEIT | | | | |
| | | | | TLVTVFKTIRGK | | | | |
| | | | | APAYLASLFKW | | | | |
| | | | | EAPPTMSVRPTR | | | | |
| | | | | SEVKRLRDYDP | | | | |
| | | | | HLLWCPPARVI | | | | |
| | | | | AFRNSLQSYGPF | | | | |
| | | | | LWNSLPLKQRQ | | | | |
| | | | | LLSLRTFKKFIE | | | | |
| | | | | N | | | | |
| L2-5_GA | Gastero- | RT and | NNNNNN | (SEQ ID NO: 408) | | | (SEQ | (SEQ ID |
| | steus | EN, | NNNNNN | MRRLLLLFLIMC | | | ID NO: | NO: |
| | aculeatus | signal | NNNNNN | LTPSPVPVRISSR | | | 708) | 808) |
| | | peptide | NNNNNN | RYYRPRARSAL | | | CAGT | TAAAG |
| | | | NNNNNN | YRNLSSLSYPTR | | | GTGC | ACTAA |
| | | | NNNNNN | STHVQHLVTGG | | | ATCT | CAAAT |
| | | | NNNNNN | LWNCQSATRKA | | | CTTCT | TGTAG |
| | | | NNNNNN | DFISGFAIQQSL | | | ACAA | CACTT |
| | | | NNNNNN | DFLALTETWITP | | | GGCC | AAATT |
| | | | NNNNNN | ENTSTPAALSSA | | | ACCA | GTACT |
| | | | NNNNNN | FSFSHTPRPTGR | | | GACA | TGTAA |
| | | | NNNNNN | GGGTGLLISPK | | | TCCA | CGTCA |
| | | | NNNNNN | WSFSLYPLPPST | | | GGCG | CTCAT |
| | | | NNNNNN | PLSFEFHAVTIT | | | ACCT | CTATA |
| | | | NNNNNN | HPVQLTIIVLYR | | | GAAA | GCAAA |
| | | | NNNNNN | PPGSLGHFLEEL | | | TCGG | TTGTA |
| | | | NNNNnn | DILLSNFPENGP | | | ACTA | AATTG |
| | | | nnnnnnnn | PLILLGDFNIQTE | | | AGTA | GCTTA |
| | | | nnnnnnnn | KSSDLLHLLSSF | | | TCTCT | TTTGA |
| | | | nnnnnnnn | ALSLSPSPPTHK | | | TTCTT | GGAA |
| | | | nnnnnnnn | AGNHLDYIFTR | | | TAAA | ATTGC |
| | | | nnnnnnnn | NCSTTNLSVTPL | | | CCAA | ACTTT |
| | | | nnnnnnnn | HVSDHFFISYSL | | | GCTA | CTTGT |
| | | | nnnnnnnn | PLSITNKPPSLTN | | | GTCT | TTCTT |
| | | | nnnnnnnn | SIPARRNIRSLSP | | | GCTA | GTTCT |
| | | | nnnnnnnn | SSLASSVLSALP | | | AATT | CCTGA |
| | | | nnnnnnnn | STDSFSLLHPNA | | | TGCT | GTTTG |
| | | | nnnnnnnn | AAETLLSTLSSS | | | GTTG | TACCC |
| | | | nnnnnnnn | LDSLCPLTTRRT | | | ATTCT | TATGG |
| | | | nn | GKSPPAPWLSQ | | | AGTG | TTGAA |
| | | | | PVRAMRATMR | | | TCTTA | TGCAC |
| | | | | ASERRWRKYKR | | | AGTT | TTATT |
| | | | | PDDLLEFQSLLS | | | AATT | GTACG |
| | | | | SFSASISAAKSSF | | | TGAT | TCGCT |
| | | | | YQSKIESSFSNP | | | TGCT | TTGGA |
| | | | | KKLFSIFSNLLEP | | | GATT | TAAAA |
| | | | | PTPPPPSTLLPG | | | TGCTT | GCGTC |
| | | | | DFVNYFTKKIA | | | TAGT | AGCTA |
| | | | | DIRSSFSNPPPTS | | | TTTTG | AATGA |
| | | | | RVPPTSPLSPSLS | | | TCGT | CATGT |
| | | | | SFTALSPNQILT | | | CTCA | AATGT |
| | | | | LVTSARPTTCPL | | | CGGC | AATGT |
| | | | | DPIPSHLLQSIAP | | | CAGT | AATGT |
| | | | | DLLPFLTCLINN | | | TCTCT | AATG |
| | | | | ALSSGCFPNSLK | | | TTGTT | |
| | | | | EARVNPLLKKP | | | TGAA | |
| | | | | TLNPSEENNYRP | | | GTTA | |
| | | | | VSLLPFLSKTLE | | | TCTTG | |
| | | | | RAIFNQLSSYLH | | | GTTG | |
| | | | | CNNLLDPHQSG | | | CTAG | |
| | | | | FKAGHSTETAL | | | TTTGC | |
| | | | | LAVSEQLHTAR | | | TCAA | |
| | | | | AASLSSVLILLD | | | GCTC | |
| | | | | LSAAFDTVNHQI | | | TTTCA | |
| | | | | LISSLQELGVTG | | | GTCTT | |
| | | | | SALSLLSSYLDG | | | TTAG | |
| | | | | RTYRVTWRGSV | | | TGCC | |
| | | | | SEPCPLTTGVPQ | | | AGTT | |
| | | | | GSVLGPLLFSLY | | | TGTG | |
| | | | | TNSLGAVIRSHG | | | TTCTA | |
| | | | | FSYHSYADDTQ | | | GATT | |
| | | | | LILSFPHSDTQV | | | CTAC | |
| | | | | AARISACLTDIS | | | TATT | |
| | | | | QWMSAHHLKIN | | | AAGT | |
| | | | | PDKTELLLFPGK | | | TAAC | |
| | | | | DSLTQDLTVNF | | | TGCC | |
| | | | | GNSVLTPTSTAK | | | AGTG | |
| | | | | NLGVTLDSQLS | | | TGCC | |
| | | | | LTPNITATTRSC | | | CTCCT | |
| | | | | RYTLYNIRRIRP | | | AACT | |
| | | | | LLTQKAAQVLI | | | CTGC | |
| | | | | QALVISRLDYC | | | TAGG | |
| | | | | NSLLAGLPATAI | | | TTTTG | |
| | | | | RPLQLIQNAAA | | | ACCT | |
| | | | | RLVFNLPKFSHT | | | GATC | |
| | | | | TPLLRSLHWLP | | | TAAG | |
| | | | | VAARIQFKTLVL | | | TCTGT | |
| | | | | TYHAVNGSGPA | | | TTTGC | |
| | | | | YIQDMVKPYIPT | | | CTTGT | |
| | | | | RTLRSASAKLL | | | TTTCT | |
| | | | | VPPSLRAKHSTR | | | GAAG | |
| | | | | SRLFAVLAPKW | | | CAAA | |
| | | | | WNELSEDTRTA | | | TAAG | |
| | | | | ESLHIFRRKLKT | | | ACTT | |
| | | | | HLFRLYLD | | | GTAT | |
| | | | | | | | CCTT | |
| | | | | | | | AAAC | |
| | | | | | | | TCTC | |
| | | | | | | | ATTTT | |
| | | | | | | | GTCA | |
| | | | | | | | AAAC | |
| | | | | | | | ACCA | |
| | | | | | | | CATG | |
| | | | | | | | GTGT | |
| | | | | | | | TTGTT | |
| | | | | | | | CACT | |
| | | | | | | | GATT | |
| | | | | | | | AGGG | |
| | | | | | | | TGTC | |
| | | | | | | | AAGC | |
| | | | | | | | TATT | |
| | | | | | | | GGTG | |
| | | | | | | | CTTTG | |
| | | | | | | | CATTT | |
| | | | | | | | TGAG | |
| | | | | | | | GAGT | |
| | | | | | | | TTCTG | |
| | | | | | | | TCGA | |
| | | | | | | | GGCC | |
| | | | | | | | AATC | |
| | | | | | | | GACC | |
| | | | | | | | TTTTG | |
| | | | | | | | TCTCC | |
| | | | | | | | TAAA | |
| | | | | | | | CGAC | |
| | | | | | | | GGGG | |
| | | | | | | | GAGC | |
| | | | | | | | GGCC | |
| | | | | | | | AGCG | |
| | | | | | | | CAGC | |
| | | | | | | | CGCA | |
| | | | | | | | GCCG | |
| | | | | | | | ACTT | |
| | | | | | | | CCAC | |
| | | | | | | | TAAC | |
| | | | | | | | GAGG | |
| | | | | | | | GAGT | |
| | | | | | | | ATTT | |
| | | | | | | | AACT | |
| | | | | | | | AGAA | |
| | | | | | | | ATCG | |
| | | | | | | | TGGG | |
| | | | | | | | AAGC | |
| | | | | | | | GTTA | |
| | | | | | | | GCTT | |
| | | | | | | | ATCC | |
| | | | | | | | TCGC | |
| | | | | | | | AGCA | |
| | | | | | | | CGAA | |
| | | | | | | | GACG | |
| | | | | | | | AGCA | |
| | | | | | | | GAAC | |
| | | | | | | | AAAG | |
| | | | | | | | ACCA | |
| | | | | | | | GGGA | |
| | | | | | | | GTCTT | |
| | | | | | | | TCGT | |
| | | | | | | | GGAG | |
| | | | | | | | TCAA | |
| | | | | | | | GACG | |
| | | | | | | | AGAG | |
| | | | | | | | ACAA | |
| | | | | | | | AGAC | |
| | | | | | | | CAGG | |
| | | | | | | | GAGT | |
| | | | | | | | CTTTC | |
| | | | | | | | GTGG | |
| | | | | | | | AGTC | |
| | | | | | | | AAGA | |
| | | | | | | | CGAG | |
| | | | | | | | CAGA | |
| | | | | | | | ACAA | |
| | | | | | | | AGAC | |
| | | | | | | | AAGG | |
| | | | | | | | GAGT | |
| | | | | | | | CTTTC | |
| | | | | | | | GTGG | |
| | | | | | | | CAGT | |
| | | | | | | | CTTC | |
| | | | | | | | GGGG | |
| | | | | | | | CGGC | |
| | | | | | | | TGA | |
| RTE-3_BF | Branch- | RT and | NTNNNN | (SEQ ID NO: 409) | — | | (SEQ | (SEQ ID |
| | iostoma | EN | NNNNNN | MSGTPRVAFDS | | | ID NO: | NO: |
| | floridae | | NTNNNN | GKDLRNPIGQSP | | | 709) | 809) |
| | | | NNNNNN | PALSRAAPGQL | | | TCTGT | TGACC |
| | | | NNNNNN | GTDPSRSACFIG | | | AAAT | TGATA |
| | | | NNNNNG | CLELRVLLDKW | | | GGCT | CAGAG |
| | | | NNNNNN | IVCRAPDKQESK | | | GTGT | CGCTA |
| | | | NNNNNN | EKRRQKTQPIRI | | | GATG | CCATC |
| | | | NNNNNN | GSWNVRTMRT | | | CGTC | ATCTG |
| | | | NNNNNN | GLSDDLTVIEDI | | | TAGT | GAAA |
| | | | NNNNNN | RKTAAIDRELY | | | GTGT | GATGG |
| | | | NNNNNN | RLNIDIVALQET | | | AGGC | AAGG |
| | | | TNNNTN | RLPDSGSLKEDS | | | GTAG | ATGCC |
| | | | NNNNNN | YTFFWQGKGM | | | CGGT | TACTA |
| | | | NNNNNA | EETREHGVGFA | | | AGTG | C |
| | | | NNNNNN | VRNTLLHMIEPP | | | TGCTT | |
| | | | NNNNnn | TGGTERIITLRLS | | | GCCA | |
| | | | nnnnnnnn | THEGPVNLLCV | | | CTTCT | |
| | | | nntnnnnn | YAPTLQATSEV | | | CGCT | |
| | | | nnnnnnnn | KDQFYGQLDSA | | | TTCA | |
| | | | ngngnnnn | IKKIPVSEHIFIL | | | GCCC | |
| | | | nntnnnnn | GDFNARVGTDQ | | | TCAC | |
| | | | cnnnnnnn | ESWQTVLGHHG | | | CGCT | |
| | | | ntnnnnnn | IGKMNENGQRL | | | GGCT | |
| | | | nnnnnnnn | LELCCYHNLCV | | | AGCG | |
| | | | tnnnnnnn | TNTFFQNKAIHK | | | GAGC | |
| | | | nnnnnnan | ASWRHPRSQRW | | | TGCA | |
| | | | nnnnnnnn | HQLDLVITRRTS | | | TGCA | |
| | | | nnnnnnnn | LNSVCNTRAYH | | | GCAC | |
| | | | nn | SADCDTDHSLIA | | | TGCG | |
| | | | (SEQ ID | ARIKLRPKKLH | | | AAAA | |
| | | | NO: | HMKKKGQPKID | | | GAGA | |
| | | | 15407) | VSKTMLPDRNQ | | | GCCG | |
| | | | | KFLECLEGTLN | | | AGTG | |
| | | | | NIQPQDAEHRW | | | CGTA | |
| | | | | ETLSKTIYSAAA | | | AGTC | |
| | | | | QSYGKKERKNT | | | TCTCC | |
| | | | | DWFEAYISELEP | | | TGCC | |
| | | | | VMDTKRKALVS | | | AGTA | |
| | | | | YKQNPSSQNLQ | | | CATA | |
| | | | | ALKAARQEAQR | | | GCCT | |
| | | | | ASRRCANNYWL | | | CTCC | |
| | | | | LLSERIQLASAT | | | ACAG | |
| | | | | GDIRRMYEGIK | | | CAAG | |
| | | | | QATGKPIKKSAP | | | TCCC | |
| | | | | LKAKSGEIITDK | | | CATT | |
| | | | | DKQMARWVEH | | | GCAG | |
| | | | | YLDIYSTENSVS | | | TGCC | |
| | | | | QDALDNIEDFSV | | | TCCTC | |
| | | | | LAELDADPTIEE | | | GTGG | |
| | | | | LSKAIDSMSNG | | | CTAC | |
| | | | | KAPGEDNIPAEII | | | AGAC | |
| | | | | KSGKSVLLEPLH | | | GGAA | |
| | | | | ELLRLCWKEGK | | | ACTG | |
| | | | | VPQSMRNSKIV | | | GGCA | |
| | | | | TLYKNKGDRTD | | | CCGT | |
| | | | | CNSYRGISLLSI | | | AAGG | |
| | | | | VGKVFAKVVLT | | | CCCC | |
| | | | | RLQVLADRVYP | | | AGGC | |
| | | | | ESQCGFRAERST | | | TAAA | |
| | | | | TDMIFSVRQLQE | | | CTGC | |
| | | | | KCREQQRPLYIA | | | AGGG | |
| | | | | FIDLTKAFDLVS | | | AGCT | |
| | | | | RRGLFQLLRKIG | | | CGGA | |
| | | | | CPPQLLDIIISFH | | | GGAG | |
| | | | | EDMKGVVSFDG | | | GTCT | |
| | | | | ETSEPFAIRSGV | | | GGCC | |
| | | | | KQGCVLAPTLF | | | CCCA | |
| | | | | GIFFSLLLKSAF | | | GACG | |
| | | | | GHSTQGVHLHT | | | CACG | |
| | | | | RSDGKLFNLAR | | | GCAT | |
| | | | | LRAKTKVRSVLI | | | GCAT | |
| | | | | RDMLFADDAAL | | | GGCC | |
| | | | | VAHVEDELQQL | | | CACC | |
| | | | | LNQFAHACSEF | | | GGCG | |
| | | | | ALTISIKKTVVM | | | TGTG | |
| | | | | GQDVPQPPVVTI | | | GACA | |
| | | | | GSEVLEVTDHF | | | CGCC | |
| | | | | TYLGSTVTSNLS | | | CTGA | |
| | | | | LDKEIDRRIARA | | | TGCC | |
| | | | | AGVMTKLGTRV | | | TGCG | |
| | | | | WNNSHLTLNTK | | | AACC | |
| | | | | LEVYRSCVLSTL | | | AGAC | |
| | | | | LYGSETWTTYA | | | CCCC | |
| | | | | KQENRLESFHL | | | AGCT | |
| | | | | RCLRRILGISWR | | | ATGG | |
| | | | | DRVPNTTVLER | | | GCAA | |
| | | | | SCSLSIHLLLCQ | | | ATAG | |
| | | | | RRLRWLGHVSR | | | CACG | |
| | | | | MKDGRIPKDILF | | | GGTA | |
| | | | | GELATGKRPVG | | | GACG | |
| | | | | RPALRFRDVCK | | | GAGC | |
| | | | | RDLKLTDIDPAS | | | TCGT | |
| | | | | WEQIAADRNR | | | CAGC | |
| | | | | WRHTVKDGLA | | | CTTG | |
| | | | | KGQERRTEHLE | | | GATG | |
| | | | | SRRRKRKEKPQ | | | GCAG | |
| | | | | QGNPSAFICPNC | | | TTCGT | |
| | | | | GRDCHARIGLQ | | | CTAG | |
| | | | | SHSRRCQPP | | | GGGA | |
| | | | | | | | AGGA | |
| | | | | | | | AAAC | |
| | | | | | | | CCTG | |
| | | | | | | | ATTC | |
| | | | | | | | AAAA | |
| | | | | | | | ACCT | |
| | | | | | | | CCGC | |
| | | | | | | | TGCC | |
| | | | | | | | TTGC | |
| | | | | | | | GGCT | |
| | | | | | | | ATAC | |
| | | | | | | | CCAG | |
| | | | | | | | TCCT | |
| | | | | | | | GGGA | |
| | | | | | | | AAGG | |
| | | | | | | | CTAC | |
| | | | | | | | GGGA | |
| | | | | | | | GTTA | |
| | | | | | | | ACCC | |
| | | | | | | | AGAG | |
| | | | | | | | AGAA | |
| | | | | | | | AATC | |
| | | | | | | | CGGA | |
| | | | | | | | GTGG | |
| | | | | | | | AGTA | |
| | | | | | | | CGTG | |
| | | | | | | | AGGC | |
| | | | | | | | GGTT | |
| | | | | | | | GGCT | |
| | | | | | | | GTCA | |
| | | | | | | | AACT | |
| | | | | | | | CTGT | |
| | | | | | | | CATC | |
| | | | | | | | CTTCC | |
| | | | | | | | GGCA | |
| | | | | | | | ACTC | |
| | | | | | | | CTGC | |
| | | | | | | | AGCC | |
| | | | | | | | AAAC | |
| | | | | | | | CAAC | |
| | | | | | | | GCCA | |
| | | | | | | | AGTG | |
| | | | | | | | TCAC | |
| | | | | | | | GCCT | |
| | | | | | | | CGCG | |
| | | | | | | | TTCCC | |
| | | | | | | | TTGG | |
| | | | | | | | ACCA | |
| | | | | | | | CGTC | |
| | | | | | | | GGTG | |
| | | | | | | | AGGT | |
| | | | | | | | CGAG | |
| | | | | | | | AGGG | |
| | | | | | | | GGGT | |
| | | | | | | | CCTG | |
| | | | | | | | TTGT | |
| | | | | | | | GTTTT | |
| | | | | | | | TGGG | |
| | | | | | | | CAGC | |
| | | | | | | | GCAG | |
| | | | | | | | GTCC | |
| | | | | | | | TCCA | |
| | | | | | | | TAAA | |
| | | | | | | | CCTG | |
| | | | | | | | CCCA | |
| | | | | | | | GGCT | |
| | | | | | | | AGCG | |
| | | | | | | | CTCT | |
| | | | | | | | GGAG | |
| | | | | | | | AGGC | |
| | | | | | | | CACT | |
| | | | | | | | CCAG | |
| | | | | | | | TCGC | |
| | | | | | | | CCCC | |
| | | | | | | | ATCA | |
| | | | | | | | CTGG | |
| | | | | | | | GGGT | |
| | | | | | | | GAGA | |
| | | | | | | | AACA | |
| | | | | | | | AACC | |
| | | | | | | | GGGA | |
| | | | | | | | GACA | |
| | | | | | | | GCAG | |
| | | | | | | | TTTAC | |
| | | | | | | | GGGT | |
| | | | | | | | TATA | |
| | | | | | | | AGTC | |
| | | | | | | | CTTG | |
| | | | | | | | CTAA | |
| | | | | | | | ATTG | |
| | | | | | | | ACGT | |
| | | | | | | | AA | |
| RTE-25_ | Locusta | RT and | NNNNNN | (SEQ ID NO: 410) | | | (SEQ | (SEQ ID |
| LMi | migratoria | EN | NNNNNN | MPRKNWNCGR | | | ID NO: | NO: |
| | | | NNNNNN | RTGDEKRKMM | | | 710) | 810) |
| | | | NNNNAN | FGCWNVQGIST | | | CCCG | TAGTG |
| | | | NNNNNN | KIDLLPAELDMF | | | TGTG | TAAAA |
| | | | NNNNNN | NIDVVVLSETKR | | | GAGT | CCTTA |
| | | | NNNANN | KGKGEEELDNY | | | TTGCT | TGTAC |
| | | | NNNCNN | VHIWSGVSKAV | | | GGTC | TAGGT |
| | | | NANNNN | RAKAGVSIMIQ | | | TTCC | GTATT |
| | | | NNNNNN | KKWKKRITNWT | | | ATCG | CATTT |
| | | | NNNNNN | FINERIITVEMTL | | | GGCA | CTGGG |
| | | | NNNNNN | FAREVVIIGVYA | | | CCTC | CGTAT |
| | | | NNNNNN | PTNDTKDKEKD | | | CCCA | TAGTA |
| | | | NNNNNN | AFWDTLRETIEK | | | GGTG | TGTTG |
| | | | NNNNNN | IPRRKELIIMGD | | | GCGG | GAGGT |
| | | | NNNNNN | MNGRVGIRESC | | | ATAG | AAACC |
| | | | CNNNnnn | KIVGKHGEAEY | | | GGGA | TCTGT |
| | | | nnnnnnnn | NDNGERLIDICA | | | ATGC | AATGA |
| | | | annnnnnn | QFDLKITNTFFK | | | TCAC | GGACA |
| | | | nnnnnnan | HKDIHKYTWQQ | | | CAGA | ATCTC |
| | | | tnntnnnn | NTKELRSIIDYIII | | | TATG | AATAA |
| | | | nnnnnnnn | RQTSSFKAADV | | | GTGG | TAAAA |
| | | | nnnnannn | RSYRGAQCGSD | | | GTAC | TAAAA |
| | | | nnnnnnnn | HYLVKMKSFWP | | | CGGG | TAAA |
| | | | nngnnnnn | WKNATNDTSNI | | | GAAA | |
| | | | nannnnnt | NKMNCSEKVQ | | | TAAA | |
| | | | nnnnnnan | NVHFNIDSLQDE | | | ATAC | |
| | | | nnnnnnnn | SIRTFFKARMER | | | CCGG | |
| | | | nnannnnn | TLDESFEGSTEEI | | | GGTG | |
| | | | n | YEYIKTKVKNV | | | GACC | |
| | | | (SEQ ID | ASEVLGIKENNP | | | AAAA | |
| | | | NO: | KRAAEWWSEEI | | | CCAG | |
| | | | 15408) | ETSVKEKRNAF | | | CAAC | |
| | | | | VQWLNDKSEGT | | | TGCT | |
| | | | | RSKYKEKKNEV | | | GCCT | |
| | | | | EKKIRLAKNEA | | | TGTA | |
| | | | | WERTCANVNSK | | | GTAT | |
| | | | | LGFGRAKEAWS | | | GATA | |
| | | | | VLKALRQDTKG | | | TTGG | |
| | | | | KSNLQLVTQKE | | | CTTAT | |
| | | | | WEEYFKKLLNE | | | CAAA | |
| | | | | DRDEYLEEGTV | | | GGCT | |
| | | | | EENEHHDDEILI | | | AAAG | |
| | | | | SESEVLQVLRTG | | | GAAG | |
| | | | | KNGKSPGPGNI | | | AAAA | |
| | | | | NMEFLKYGGDK | | | CCTT | |
| | | | | IVKLILQLFNKM | | | GAAT | |
| | | | | LHGDSVPKEMK | | | ACAA | |
| | | | | LGYISTIFKKGD | | | ATTA | |
| | | | | RKICSNYRGICV | | | CCTG | |
| | | | | TNTLMRIFGKII | | | GTCC | |
| | | | | KNKLEKNFRTQ | | | TCCA | |
| | | | | QEQCGFTAGRS | | | GGTT | |
| | | | | CVDHIFTLRQIL | | | GGGG | |
| | | | | EKHREKSKNVG | | | GTTG | |
| | | | | LIFIDLEKAYDT | | | TGCA | |
| | | | | VPRKLLWRALH | | | GTGG | |
| | | | | RANINTSLIKIIE | | | GCCA | |
| | | | | QMYKDNICQVK | | | GCTC | |
| | | | | IGNTLSQKFRTS | | | CTCA | |
| | | | | KGLLQGCPMSP | | | CTCA | |
| | | | | TLFKIYIDICLRT | | | CATA | |
| | | | | WSQKCNSMGLE | | | AAAA | |
| | | | | IRDGVYLHHLLF | | | TATA | |
| | | | | ADDQVVIAQDG | | | AAAT | |
| | | | | EDANYMCNQL | | | GCTA | |
| | | | | AIAYKNWGLKI | | | AAAA | |
| | | | | NYQKTEYLTND | | | ACCT | |
| | | | | PHELRIEGKKIK | | | AATA | |
| | | | | KVNTFCYLGSIL | | | AT | |
| | | | | ETEGKSDSEINK | | | | |
| | | | | RISSGRKVIGML | | | | |
| | | | | NSVLWSRNVM | | | | |
| | | | | NRTKKIIYKSIFE | | | | |
| | | | | SAVLYGAETWT | | | | |
| | | | | INQKHTKKLQA | | | | |
| | | | | LEMDFWRRSAR | | | | |
| | | | | ISRKEKRRNTEV | | | | |
| | | | | IKRMEIIERIDEV | | | | |
| | | | | MDRKKLRWYG | | | | |
| | | | | HVRRMEDTRIP | | | | |
| | | | | KLVLEWQPEGR | | | | |
| | | | | RRRGRPVTTWI | | | | |
| | | | | KNVQLTMNRLG | | | | |
| | | | | AEEEDTQDRHT | | | | |
| | | | | WRNIVNN | | | | |

### Retrotransposon Discovery Tools

As the result of repeated mobilization over time, transposable elements in genomic DNA often exist as tandem or interspersed repeats (Jurka Curr Opin Struct Biol 8, 333-337 (1998)). Tools capable of recognizing such repeats can be used to identify new elements from genomic DNA and for populating databases, e.g., Repbase (Jurka et al Cytogenet Genome Res 110, 462-467 (2005)). One such tool for identifying repeats that may comprise transposable elements is RepeatFinder (Volfovsky et al Genome Biol 2 (2001)), which analyzes the repetitive structure of genomic sequences. Repeats can further be collected and analyzed using additional tools, e.g., Censor (Kohany et al BMC Bioinformatics 7, 474 (2006)). The Censor package takes genomic repeats and annotates them using various BLAST approaches against known transposable elements. An all-frames translation can be used to generate the ORF(s) for comparison.

Other exemplary methods for identification of transposable elements include RepeatModeler2, which automates the discovery and annotation of transposable elements in genome sequences (Flynn et al bioRxiv (2019)). In addition to accomplishing this via available packages like Censor, one can perform an all-frames translation of a given genome or sequence and annotate with a protein domain tool like InterProScan, which tags the domains of a given amino acid sequence using the InterPro database (Mitchell et al. Nucleic Acids Res 47, D351-360 (2019)), allowing the identification of potential proteins comprising domains associated with known transposable elements.

Retrotransposons can be further classified according to the reverse transcriptase domain using a tool such as RTclass1 (Kapitonov et al Gene 448, 207-213 (2009)).

### Polypeptide Component of Gene Modifying System

### RT Domain

In certain aspects of the present invention, the reverse transcriptase domain of the gene modifying system is based on a reverse transcriptase domain of an APE-type or RLE-type non-LTR retrotransposon, or of a PLE-type retrotransposon. A wild-type reverse transcriptase domain of an APE-type, RLE-type, or PLE-type retrotransposon can be used in a gene modifying system or can be modified (e.g., by insertion, deletion, or substitution of one or more residues) to alter the reverse transcriptase activity for target DNA sequences. In some embodiments, the reverse transcriptase is altered from its natural sequence to have altered codon usage, e.g. improved for human cells. In some embodiments, the reverse transcriptase domain is a heterologous reverse transcriptase from a different LTR-retrotransposon, non-LTR retrotransposon, or other source. In certain embodiments, a gene modifying system includes a polypeptide that comprises a reverse transcriptase domain of a RTE (e.g., RTE-1_MD, RTE-3_BF, and RTE-25_LMi), CR1 (e.g., CR1-1_PH), Crack (e.g., Crack-28_RF), L2 (e.g., L2-2_Dre and L2-5_GA), and Vingi (e.g., Vingi-1_Acar) retrotransposon.

In certain embodiments, a gene modifying system includes a polypeptide that comprises a reverse transcriptase domain of a retrotransposon listed in Table 10, Table 11, Table X, Table Z1, Table Z2, or Table 3A or 3B of PCT Pub. No.: WO/2021/178717, which are incorporated herein by reference as they relate to domains from retrotransposons.

In certain embodiments, a gene modifying system includes a polypeptide that comprises a reverse transcriptase domain of a retrotransposon listed in Table R1. In some embodiments, the amino acid sequence of the reverse transcriptase domain of a gene modifying system is at least about 50%, at least about 60%, at least about 70%, at least about 80%, at least about 85%, at least about 90%, at least about 95%, at least about 96%, at least about 97%, at least about 98%, at least about 99% identical to the amino acid sequence of a reverse transcriptase domain of a retrotransposon whose DNA sequence is referenced in Table R1. Reverse transcriptase domains can be identified, for example, based upon homology to other known reverse transcription domains using routine tools as Basic Local Alignment Search Tool (BLAST). In some embodiments, reverse transcriptase domains are modified, for example by site-specific mutation. In some embodiments, the reverse transcriptase domain is engineered to bind a heterologous template RNA.

In some embodiments, a polypeptide (e.g., RT domain) comprises an RNA-binding domain, e.g., that specifically binds to an RNA sequence. In some embodiments, a template RNA comprises an RNA sequence that is specifically bound by the RNA-binding domain.

In some embodiments, the RT domain forms a dimer (e.g., a heterodimer or homodimer). In some embodiments, the RT domain is monomeric. In some embodiments, an RT domain naturally functions as a monomer or as a dimer (e.g., heterodimer or homodimer). In some embodiments, an RT domain naturally functions as a monomer. Naturally heterodimeric RT domains may, in some embodiments, also be functional as homodimers. In some embodiments, dimeric RT domains are expressed as fusion proteins, e.g., as homodimeric fusion proteins or heterodimeric fusion proteins. In some embodiments, the RT function of the system is fulfilled by multiple RT domains (e.g., as described herein). In further embodiments, the multiple RT domains are fused or separate, e.g., may be on the same polypeptide or on different polypeptides.

In some embodiment, a gene modifying polypeptide described herein comprises an RNase H domain, e.g., wherein the RNase H domain may be part of the RT domain. In some embodiments, an RT domain (e.g., as described herein) comprises an RNase H domain, e.g., an endogenous RNAse H domain or a heterologous RNase H domain. In some embodiments, an RT domain (e.g., as described herein) lacks an RNase H domain. In some embodiments, an RT domain (e.g., as described herein) comprises an RNase H domain that has been added, deleted, mutated, or swapped for a heterologous RNase H domain. In some embodiments, mutation of an RNase H domain yields a polypeptide exhibiting lower RNase activity, e.g., as determined by the methods described in Kotewicz et al. Nucleic Acids Res 16(1):265-277 (1988) (incorporated herein by reference in its entirety), e.g., lower by at least 10%, 20%, 30%, 40%, 50%, 60%, 70%, 80%, or 90% compared to an otherwise similar domain without the mutation. In some embodiments, RNase H activity is abolished.

In some embodiments, an RT domain is mutated to increase fidelity compared to an otherwise similar domain without the mutation. For instance, in some embodiments, a YADD (SEQ ID NO: 15409) or YMDD motif (SEQ ID NO: 15410) in an RT domain (e.g., in a reverse transcriptase) is replaced with YVDD (SEQ ID NO: 15411). In embodiments, replacement of the YADD (SEQ ID NO: 15409) or YMDD (SEQ ID NO: 15410) or YVDD (SEQ ID NO: 15411) results in higher fidelity in retroviral reverse transcriptase activity (e.g., as described in Jamburuthugoda and Eickbush J Mol Biol 2011; incorporated herein by reference in its entirety).

### Endonuclease Domain:

In some embodiments, the polypeptide comprises an endonuclease domain (e.g., a heterologous endonuclease domain). In certain embodiments, the endonuclease/DNA binding domain of an APE-type retrotransposon, the endonuclease domain of an RLE-type retrotransposon, or the endonuclease domain of a PLE-type retrotransposon can be used or can be modified (e.g., by insertion, deletion, or substitution of one or more residues) in a gene modifying system described herein. In some embodiments, the endonuclease domain or endonuclease/DNA binding domain is altered from its natural sequence to have altered codon usage, e.g. improved for human cells. In some embodiments, the endonuclease element is a heterologous endonuclease element. The amino acid sequence of an endonuclease domain of a gene modifying system described herein may be at least about 50%, at least about 60%, at least about 70%, at least about 80%, at least about 85%, at least about 90%, at least about 95%, at least about 96%, at least about 97%, at least about 98%, at least about 99% identical to the amino acid sequence of an endonuclease domain of a retrotransposon whose DNA sequence is referenced in Table X, Z1, Z2, 3A, or 3B of PCT Pub. No: WO/2021/178717, which are incorporated herein by reference as they relate to domains from retrotransposons.

In certain embodiments, a gene modifying system includes a polypeptide that comprises an endonuclease domain of a retrotransposon listed in Table R1. In some embodiments, the amino acid sequence of the endonuclease domain of a gene modifying system is at least about 50%, at least about 60%, at least about 70%, at least about 80%, at least about 85%, at least about 90%, at least about 95%, at least about 96%, at least about 97%, at least about 98%, at least about 99% identical to the amino acid sequence of a endonuclease domain of a retrotransposon whose DNA sequence is referenced in Table R1. Endonuclease domains can be identified, for example, based upon homology to other known endonuclease domains using tools as Basic Local Alignment Search Tool (BLAST).

In some embodiments, a gene modifying polypeptide possesses the function of DNA target site cleavage via an endonuclease domain. In some embodiments, the endonuclease domain is also a DNA-binding domain. In some embodiments, the endonuclease domain is also a template nucleic acid (e.g., template RNA) binding domain. In certain embodiments, the endonuclease/DNA binding domain of an APE-type retrotransposon or the endonuclease domain of an RLE-type retrotransposon can be used or can be modified (e.g., by insertion, deletion, or substitution of one or more residues) in a gene modifying system described herein.

### Template Nucleic Acid Binding Domain:

A gene modifying polypeptide typically contains regions capable of associating with the template nucleic acid (e.g., template RNA). In some embodiments, the template nucleic acid binding domain is an RNA binding domain. In some embodiments, the RNA binding domain is a modular domain that can associate with RNA molecules containing specific signatures, e.g., structural motifs, e.g., secondary structures present in the 3′ UTR in non-LTR retrotransposons. In other embodiments, the template nucleic acid binding domain (e.g., RNA binding domain) RNA binding domain is contained within the reverse transcription domain, e.g., the reverse transcriptase-derived component has a known signature for RNA preference, e.g., secondary structures present in the 3′ UTR in non-LTR retrotransposons.

### DNA Binding Domain:

In certain aspects, the DNA-binding domain of a gene modifying polypeptide described herein is selected, designed, or constructed for binding to a desired host DNA target sequence. In certain embodiments, the DNA-binding domain of the engineered retrotransposon is a heterologous DNA-binding protein or domain relative to a native retrotransposon sequence. In certain embodiments, the heterologous DNA-binding domain is a DNA binding domain of a retrotransposon described in Table R1 herein or in Table X, Table Z1, Table Z2, or Table 3A or 3B of PCT Pub. No.: WO/2021/178717. In some embodiments, DNA binding domains can be identified based upon homology to other known DNA binding domains using tools as Basic Local Alignment Search Tool (BLAST). In still other embodiments, DNA-binding domains are modified, for example by site-specific mutation. In some embodiments, the DNA binding domain is altered from its natural sequence to have altered codon usage, e.g. improved for human cells.

In embodiments, the DNA binding domain comprises one or more modifications relative to a wild-type DNA binding domain, e.g., a modification via directed evolution, e.g., phage-assisted continuous evolution (PACE).

In certain aspects of the present invention, the host DNA-binding site integrated into by the gene modifying system can be in a gene, in an intron, in an exon, an ORF, outside of a coding region of any gene, in a regulatory region of a gene, or outside of a regulatory region of a gene. In other aspects, the engineered retrotransposon may bind to one or more than one host DNA sequence. In other aspects, the engineered retrotransposon may have low sequence specificity, e.g., bind to multiple sequences or lack sequence preference.

In some embodiments, a gene modifying system is used to edit a target locus in multiple alleles. In some embodiments, a gene modifying system is designed to edit a specific allele. For example, a gene modifying polypeptide may be directed to a specific sequence that is only present on one allele, e.g., comprises a template RNA with homology to a target allele, e.g., an annealing domain, but not to a second cognate allele. In some embodiments, a gene modifying system can alter a haplotype-specific allele. In some embodiments, a gene modifying system that targets a specific allele preferentially targets that allele, e.g., has at least a 2, 4, 6, 8, or 10-fold preference for a target allele.

### Localization Sequences for Gene Modifying Systems

In certain embodiments, a gene modifying system RNA further comprises an intracellular localization sequence, e.g., a nuclear localization sequence.

The nuclear localization sequence may be an RNA sequence that promotes the import of the RNA into the nucleus. In certain embodiments, the nuclear localization signal is located on the template RNA. In certain embodiments, the retrotransposase polypeptide is encoded on a first RNA, and the template RNA is a second, separate, RNA, and the nuclear localization signal is located on the template RNA and not on an RNA encoding the retrotransposase polypeptide. While not wishing to be bound by theory, in some embodiments, the RNA encoding the retrotransposase is targeted primarily to the cytoplasm to promote its translation, while the template RNA is targeted primarily to the nucleus to promote its retrotransposition into the genome. In some embodiments, the nuclear localization signal is at the 3′ end, 5′ end, or in an internal region of the template RNA. In some embodiments the nuclear localization signal is 3′ of the heterologous sequence (e.g., is directly 3′ of the heterologous sequence) or is 5′ of the heterologous sequence (e.g., is directly 5′ of the heterologous sequence). In some embodiments, the nuclear localization signal is placed outside of the 5′ UTR or outside of the 3′ UTR of the template RNA. In some embodiments the nuclear localization signal is placed between the 5′ UTR and the 3′ UTR, wherein optionally the nuclear localization signal is not transcribed with the transgene (e.g., the nuclear localization signal is an anti-sense orientation or is downstream of a transcriptional termination signal or polyadenylation signal). In some embodiments, the nuclear localization sequence is situated inside of an intron. In some embodiments a plurality of the same or different nuclear localization signals are in the RNA, e.g., in the template RNA. In some embodiments, the nuclear localization signal is less than 5, 10, 25, 50, 75, 100, 150, 200, 250, 300, 350, 400, 450, 500, 600, 700, 800, 900, or 1000 bp in length. Various RNA nuclear localization sequences can be used. For example, Lubelsky and Ulitsky, Nature 555 (107-111), 2018 describe RNA sequences, which drive RNA localization into the nucleus. In some embodiments, the nuclear localization signal is a SINE-derived nuclear RNA localization (SIRLOIN) signal. In some embodiments, the nuclear localization signal binds a nuclear-enriched protein. In some embodiments, the nuclear localization signal binds the HNRNPK protein. In some embodiments the nuclear localization signal is rich in pyrimidines, e.g., is a C/T rich, C/U rich, C rich, T rich, or U rich region. In some embodiments, the nuclear localization signal is derived from a long non-coding RNA. In some embodiments, the nuclear localization signal is derived from MALAT1 long non-coding RNA or is the 600 nucleotide M region of MALAT1 (described in Miyagawa et al., RNA 18, (738-751), 2012). In some embodiments, the nuclear localization signal is derived from BORG long non-coding RNA or is a AGCCC motif (described in Zhang et al., Molecular and Cellular Biology 34, 2318-2329 (2014). In some embodiments, the nuclear localization sequence is described in Shukla et al., The EMBO Journal e98452 (2018). In some embodiments, the nuclear localization signal is derived from a non-LTR retrotransposon, an LTR retrotransposon, retrovirus, or an endogenous retrovirus.

In some embodiments, a polypeptide described herein comprises one or more (e.g., 2, 3, 4, 5) nuclear targeting sequences, for example, a nuclear localization sequence (NLS), e.g., as described above. In some embodiments, the NLS is a bipartite NLS. In some embodiments, an NLS facilitates the import of a protein comprising an NLS into the cell nucleus. In some embodiments, the NLS is fused to the N-terminus of a gene modifying polypeptide described herein. In some embodiments, the NLS is fused to the C-terminus of the gene modifying polypeptide. In some embodiments, a linker sequence is disposed between the NLS and the neighboring domain of the gene modifying polypeptide.

In some embodiments, an NLS comprises the amino acid sequence

(SEQ ID NO: 9)
MDSLLMNRRKFLYQFKNVRWAKGRRETYLC,
(SEQ ID NO: 10)
PKKRKVEGADKRTADGSEFESPKKKRKV,
(SEQ ID NO: 11)
RKSGKIAAIWKRPRKPKKKRKV
(SEQ ID NO: 12)
KRTADGSEFESPKKKRKV,
(SEQ ID NO: 13)
KKTELQTTNAENKTKKL,
or
(SEQ ID NO: 14)
KRGINDRNFWRGENGRKTR,
(SEQ ID NO: 15)
KRPAATKKAGQAKKKK,
(SEQ ID NO: 344)
PAAKRVKLD,
(SEQ ID NO: 349)
KRTADGSEFEKRTADGSEFESPKKKAKVE,
(SEQ ID NO: 350)
KRTADGSEFE,
(SEQ ID NO: 351)
KRTADGSEFESPKKKAKVE,
(SEQ ID NO: 4001)
AGKRTADGSEFEKRTADGSEFESPKKKAKVE,
or
a functional fragment or variant thereof.

In some embodiments, a gene modifying polypeptide comprises an NLS as comprised in SEQ ID NO: 4000 and/or SEQ ID NO: 4001, or an NLS having an amino acid sequence having at least 75%, 80%, 85%, 90%, 95%, 96%, 97%, 98%, or 99% identity thereto.

Exemplary NLS sequences are also described in PCT/EP2000/011690, the contents of which are incorporated herein by reference for their disclosure of exemplary nuclear localization sequences. In some embodiments, an NLS comprises an amino acid sequence as disclosed in Table 8. An NLS of this table may be utilized with one or more copies in a polypeptide in one or more locations in a polypeptide, e.g., 1, 2, 3 or more copies of an NLS in an N-terminal domain, between peptide domains, in a C-terminal domain, or in a combination of locations, in order to improve subcellular localization to the nucleus. Multiple unique sequences may be used within a single polypeptide. Sequences may be naturally monopartite or bipartite, e.g., having one or two stretches of basic amino acids, or may be used as chimeric bipartite sequences. Sequence references correspond to UniProt accession numbers, except where indicated as SeqNLS for sequences mined using a subcellular localization prediction algorithm (Lin et al BMC Bioinformat 13:157 (2012), incorporated herein by reference in its entirety).

TABLE 8
Exemplary nuclear localization signals for use in gene modifying systems

| Sequence | Sequence References | SEQ ID No. |
|---|---|---|
| AHFKISGEKRPSTDPGKKAK | Q76IQ7 | 223 |
| NPKKKKKKDP | | |
| AHRAKKMSKTHA | P21827 | 224 |
| ASPEYVNLPINGNG | SeqNLS | 225 |
| CTKRPRW | O88622, Q86W56, Q9QYM2, O02776 | 226 |
| DKAKRVSRNKSEKKRR | O15516, Q5RAK8, Q91YB2, Q91YB0, | 227 |
| | Q8QGQ6, O08785, Q9WVS9, Q6YGZ4 | |
| EELRLKEELLKGIYA | Q9QY16, Q9UHL0, Q2TBP1, Q9QY15 | 228 |
| EEQLRRRKNSRLNNTG | G5EFF5 | 229 |
| EVLKVIRTGKRKKKAWKR | SeqNLS | 230 |
| MVTKVC | | |
| HHHHHHHHHHHHQPH | Q63934, G3V7L5, Q12837 | 231 |
| HKKKHPDASVNFSEFSK | P10103, Q4R844, P12682, B0CM99, | 232 |
| | A9RA84, Q6YKA4, P09429, P63159, | |
| | Q08IE6, P63158, Q9YH06, B1MTB0 | |
| HKRTKK | Q2R2D5 | 233 |
| IINGRKLKLKKSRRRSSQTS | SeqNLS | 234 |
| NNSFTSRRS | | |
| KAEQERRK | Q8LH59 | 235 |
| KEKRKRREELFIEQKKRK | SeqNLS | 236 |
| KKGKDEWFSRGKKP | P30999 | 237 |
| KKGPSVQKRKKT | Q6ZN17 | 238 |
| KKKTVINDLLHYKKEK | SeqNLS, P32354 | 239 |
| KKNGGKGKNKPSAKIKK | SegNLS | 240 |
| KKPKWDDFKKKKK | Q15397, Q8BKS9, Q562C7 | 241 |
| KKRKKD | SeqNLS, Q91Z62, Q1A730, Q969P5, | 242 |
| | Q2KHT6, Q9CPU7 | |
| KKRRKRRRK | SeqNLS | 243 |
| KKRRRRARK | Q9UMS6, D4A702, Q91YE8 | 244 |
| KKSKRGR | Q9UBS0 | 245 |
| KKSRKRGS | B4FG96 | 246 |
| KKSTALSRELGKIMRRR | SeqNLS, P32354 | 247 |
| KKSYQDPEIIAHSRPRK | Q9U7C9 | 248 |
| KKTGKNRKLKSKRVKTR | Q9Z301, O54943, Q8K3T2 | 249 |
| KKVSIAGQSGKLWRWKR | Q6YUL8 | 250 |
| KKYENVVIKRSPRKRGRPR | SeqNLS | 251 |
| K | | |
| KNKKRK | SeqNLS | 252 |
| KPKKKR | ScqNLS | 253 |
| KRAMKDDSHGNSTSPKRRK | Q0E671 | 254 |
| KRANSNLVAAYEKAKKK | P23508 | 255 |
| KRASEDTTSGSPPKKSSAGP | Q9BZZ5, Q5R644 | 256 |
| KR | | |
| KRFKRRWMVRKMKTKK | SeqNLS | 257 |
| KRGLNSSFETSPKKVK | Q8IV63 | 258 |
| KRGNSSIGPNDLSKRKQRK | SeqNLS | 259 |
| K | | |
| KRIHSVSLSQSQIDPSKKVK | ScqNLS | 260 |
| RAK | | |
| KRKGKLKNKGSKRKK | O15381 | 261 |
| KRRRRRRREKRKR | Q96GM8 | 262 |
| KRSNDRTYSPEEEKQRRA | Q91ZF2 | 263 |
| KRTVATNGDASGAHRAKK | SeqNLS | 264 |
| MSK | | |
| KRVYNKGEDEQEHLPKGKK | SeqNLS | 265 |
| R | | |
| KSGKAPRRRAVSMDNSNK | Q9WVH4, O43524 | 266 |
| KVNFLDMSLDDIIIYKELE | Q9P127 | 267 |
| KVQHRIAKKTTRRRR | Q9DXE6 | 268 |
| LSPSLSPL | Q9Y261, P32182, P35583 | 269 |
| MDSLLMNRRKFLYQFKNVR | Q9GZX7 | 270 |
| WAKGRRETYLC | | |
| MPQNEYIELHRKRYGYRLD | SeqNLS | 271 |
| YHEKKRKKESREAHERSKK | | |
| AKKMIGLKAKLYHK | | |
| MVQLRPRASR | SeqNLS | 272 |
| NNKLLAKRRKGGASPKDDP | Q965G5 | 273 |
| MDDIK | | |
| NYKRPMDGTYGPPAKRHEG | O14497, A2BH40 | 274 |
| E | | |
| PDTKRAKLDSSETTMVKKK | SeqNLS | 275 |
| PEKRTKI | SeqNLS | 276 |
| PGGRGKKK | Q719N1, Q9UBP0, A2VDN5 | 277 |
| PGKMDKGEHRQERRDRPY | Q01844, Q61545 | 278 |
| PKKGDKYDKTD | Q45FA5 | 279 |
| PKKKSRK | O35914, Q01954 | 280 |
| PKKNKPE | Q22663 | 281 |
| PKKRAKV | P04295, P89438 | 282 |
| PKPKKLKVE | P55263, P55262, P55264, Q64640 | 283 |
| PKRGRGR | Q9FYS5, Q43386 | 284 |
| PKRRLVDDA | P0C797 | 285 |
| PKRRRTY | SeqNLS | 286 |
| PLFKRR | A8X6H4, Q9TXJ0 | 287 |
| PLRKAKR | Q86WB0, Q5R8V9 | 288 |
| PPAKRKCIF | Q6AZ28, O75928, Q8C5D8 | 289 |
| PPARRRRL | Q8NAG6 | 290 |
| PPKKKRKV | Q3L6L5, P03070, P14999, P03071 | 291 |
| PPNKRMKVKH | Q8BN78 | 292 |
| PPRIYPQLPSAPT | P0C799 | 293 |
| PQRSPFPKSSVKR | SeqNLS | 294 |
| PRPRKVPR | P0C799 | 295 |
| PRRRVQRKR | SeqNLS, Q5R448, Q5TAQ9 | 296 |
| PRRVRLK | Q58DJ0, P56477, Q13568 | 297 |
| PSRKRPR | Q62315, Q5F363, Q92833 | 298 |
| PSSKKRKV | SeqNLS | 299 |
| PTKKRVK | P07664 | 300 |
| QRPGPYDRP | SeqNLS | 301 |
| RGKGGKGLGKGGAKRHRK | SeqNLS | 302 |
| RKAGKGGGGHKTTKKRSA | B4FG96 | 303 |
| KDEKVP | | |
| RKIKLKRAK | A1L3G9 | 304 |
| RKIKRKRAK | B9X187 | 305 |
| RKKEAPGPREELRSRGR | O35126, P54258, Q5IS70, P54259 | 306 |
| RKKRKGK | SeqNLS, Q29243, Q62165, Q28685, | 307 |
| | O18738, Q9TSZ6, Q14118 | |
| RKKRRQRRR | P04326, P69697, P69698, P05907, | 308 |
| | P20879, P04613, P19553, P0C1J9, | |
| | P20893, P12506, P04612, Q73370, | |
| | P0CIK0, P05906, P35965, P04609, | |
| | P04610, P04614, P04608, P05905 | |
| RKKSIPLSIKNLKRKHKRKK | Q9C0C9 | 309 |
| NKITR | | |
| RKLVKPKNTKMKTKLRTNP | Q14190 | 310 |
| Y | | |
| RKRLILSDKGQLDWKK | SeqNLS, Q91Z62, Q1A730, Q2KHT6, | 311 |
| | Q9CPU7 | |
| RKRLKSK | Q13309 | 312 |
| RKRRVRDNM | Q8QPH4, Q809M7, A8C8X1, Q2VNC5, | 313 |
| | Q38SQ0, 089749, Q6DNQ9, Q809L9, | |
| | Q0A429, Q20NV3, P16509, P16505, | |
| | Q6DNQ5, P16506, Q6XT06, P26118, | |
| | Q2ICQ2, Q2RCG8, Q0A2D0, Q0A2H9, | |
| | Q9IQ46, Q809M3, Q6J847, Q6J856, | |
| | B4URE4, A4GCM7, Q0A440, P26120, | |
| | P16511, | |
| RKRSPKDKKEKDLDGAGKR | Q7RTP6 | 314 |
| RKT | | |
| RKRTPRVDGQTGENDMNK | O94851 | 315 |
| RRRK | | |
| RLPVRRRRRR | P04499, P12541, P03269, P48313, | 316 |
| | P03270 | |
| RLRFRKPKSK | P69469 | 317 |
| RQQRKR | Q14980 | 318 |
| RRDLNSSFETSPKKVK | Q8K3G5 | 319 |
| RRDRAKLR | Q9SLB8 | 320 |
| RRGDGRRR | Q80WE1, Q5R9B4, Q06787, P35922 | 321 |
| RRGRKRKAEKQ | Q812D1, Q5XXA9, Q99JF8, Q8MJG1, | 322 |
| | Q66T72, O75475 | |
| RRKKRR | Q0VD86, Q58DS6, Q5R6G2, Q9ERI5, | 323 |
| | Q6AYK2, Q6NYC1 | |
| RRKRSKSEDMDSVESKRRR | Q7TT18 | 324 |
| RRKRSR | Q99PU7, D3ZHS6, Q92560, A2VDM8 | 325 |
| RRPKGKTLQKRKPK | Q6ZN17 | 326 |
| RRRGFERFGPDNMGRKRK | Q63014, Q9DBR0 | 327 |
| RRRGKNKVAAQNCRK | SeqNLS | 328 |
| RRRKRR | Q5FVH8, Q6MZT1, Q08DH5, Q8BQP9 | 329 |
| RRRQKQKGGASRRR | SeqNLS | 330 |
| RRRREGPRARRRR | P08313, P10231 | 331 |
| RRTIRLKLVYDKCDRSCKIQ | SeqNLS | 332 |
| KKNRNKCQYCRFHKCLSVG | | |
| MSHNAIRFGRMPRSEKAKL | | |
| KAE | | |
| RRVPQRKEVSRCRKCRK | Q5RJN4, Q32L09, Q8CAK3, Q9NUL5 | 333 |
| RVGGRRQAVECIEDLLNEP | P03255 | 334 |
| GQPLDLSCKRPRP | | |
| RVVKLRIAP | P52639, Q8JMN0 | 335 |
| RVVRRR | P70278 | 336 |
| SKRKTKISRKTR | Q5RAY1, O00443 | 337 |
| SYVKTVPNRTRTYIKL | P21935 | 338 |
| TGKNEAKKRKIA | P52739, Q8K3J5, Q5RAU9 | 339 |
| TLSPASSPSSVSCPVIPASTD | SeqNLS | 340 |
| ESPGSALNI | | |
| VSKKQRTGKKIH | P52739, Q8K3J5, Q5RAU9 | 341 |
| SPKKKRKVE | | 342 |
| KRTAD GSEFE SPKKKRKVE | | 343 |
| PAAKRVKLD | | 344 |
| PKKKRKV | | 345 |
| MDSLLMNRRKFLYQFKNVR | | 346 |
| WAKGRRETYLC | | |
| SPKKKRKVEAS | | 347 |
| MAPKKKRKVGIHRGVP | | 348 |
| KRTADGSEFEKRTADGSEFE | | 349 |
| SPKKKAKVE | | |
| KRTADGSEFE | | 350 |
| KRTADGSEFESPKKKAKVE | | 351 |
| AGKRTADGSEFEKRTADGS | | 4001 |
| EFESPKKKAKVE | | |

In some embodiments, the NLS is a bipartite NLS. A bipartite NLS typically comprises two basic amino acid clusters separated by a spacer sequence (which may be, e.g., about 10 amino acids in length). A monopartite NLS typically lacks a spacer. An example of a bipartite NLS is the nucleoplasmin NLS, having the sequence KR[PAATKKAGQA]KKKK (SEQ ID NO: 15), wherein the spacer is bracketed. Another exemplary bipartite NLS has the sequence PKKKRKVEGADKRTADGSEFFSPKKKRKV (SEQ ID NO: 16). Exemplary NLSs are described in International Application WO2020051561, which is herein incorporated by reference in its entirety, including for its disclosures regarding nuclear localization sequences.

In certain embodiments, a gene modifying system polypeptide further comprises an intracellular localization sequence, e.g., a nuclear localization sequence and/or a nucleolar localization sequence. The nuclear localization sequence and/or nucleolar localization sequence may be amino acid sequences that promote the import of the protein into the nucleus and/or nucleolus, where it can promote integration of heterologous sequence into the genome. In certain embodiments, a gene modifying system polypeptide (e.g., a retrotransposase, e.g., a polypeptide according to Table R1 herein) further comprises a nucleolar localization sequence. In certain embodiments, the retrotransposase polypeptide is encoded on a first RNA, and the template RNA is a second, separate, RNA, and the nucleolar localization signal is encoded on the RNA encoding the retrotransposase polypeptide and not on the template RNA. In some embodiments, the nucleolar localization signal is located at the N-terminus, C-terminus, or in an internal region of the polypeptide. In some embodiments, a plurality of the same or different nucleolar localization signals are used. In some embodiments, the nuclear localization signal is less than 5, 10, 25, 50, 75, or 100 amino acids in length. Various polypeptide nucleolar localization signals can be used. For example, Yang et al., Journal of Biomedical Science 22, 33 (2015), describe a nuclear localization signal that also functions as a nucleolar localization signal. In some embodiments, the nucleolar localization signal may also be a nuclear localization signal. In some embodiments, the nucleolar localization signal may overlap with a nuclear localization signal. In some embodiments, the nucleolar localization signal may comprise a stretch of basic residues. In some embodiments, the nucleolar localization signal may be rich in arginine and lysine residues. In some embodiments, the nucleolar localization signal may be derived from a protein that is enriched in the nucleolus. In some embodiments, the nucleolar localization signal may be derived from a protein enriched at ribosomal RNA loci. In some embodiments, the nucleolar localization signal may be derived from a protein that binds rRNA. In some embodiments, the nucleolar localization signal may be derived from MSP58. In some embodiments, the nucleolar localization signal may be a monopartite motif. In some embodiments, the nucleolar localization signal may be a bipartite motif. In some embodiments, the nucleolar localization signal may consist of a multiple monopartite or bipartite motifs. In some embodiments, the nucleolar localization signal may consist of a mix of monopartite and bipartite motifs. In some embodiments, the nucleolar localization signal may be a dual bipartite motif. In some embodiments, the nucleolar localization motif may be a KRASSQALGTIPKRRSSSRFIKRKK (SEQ ID NO: 17). In some embodiments, the nucleolar localization signal may be derived from nuclear factor-κB-inducing kinase. In some embodiments, the nucleolar localization signal may be an RKKRKKK motif (SEQ ID NO: 18) (described in Birbach et al., Journal of Cell Science, 117 (3615-3624), 2004).

In some embodiments, a nucleic acid described herein (e.g., an RNA encoding a gene modifying polypeptide, or a DNA encoding the RNA) comprises a microRNA binding site. In some embodiments, the microRNA binding site is used to increase the target-cell specificity of a gene modifying system. For instance, the microRNA binding site can be chosen on the basis that is recognized by a miRNA that is present in a non-target cell type, but that is not present (or is present at a reduced level relative to the non-target cell) in a target cell type. Thus, when the RNA encoding the gene modifying polypeptide is present in a non-target cell, it would be bound by the miRNA, and when the RNA encoding the gene modifying polypeptide is present in a target cell, it would not be bound by the miRNA (or bound but at reduced levels relative to the non-target cell). While not wishing to be bound by theory, binding of the miRNA to the RNA encoding the gene modifying polypeptide may reduce production of the gene modifying polypeptide, e.g., by degrading the mRNA encoding the polypeptide or by interfering with translation. Accordingly, the heterologous object sequence would be inserted into the genome of target cells more efficiently than into the genome of non-target cells. A system having a microRNA binding site in the RNA encoding the gene modifying polypeptide (or encoded in the DNA encoding the RNA) may also be used in combination with a template RNA that is regulated by a second microRNA binding site, e.g., as described herein in the section entitled “Template RNA component of gene modifying system.”

In some embodiments, a polypeptide for use in any of the systems described herein can be a molecular reconstruction or ancestral reconstruction based upon the aligned polypeptide sequence of multiple retrotransposons. In some embodiments, a 5′ or 3′ untranslated region for use in any of the systems described herein can be a molecular reconstruction based upon the aligned 5′ or 3′ untranslated region of multiple retrotransposons. Based on the Accession numbers, polypeptides or nucleic acid sequences can be aligned, e.g., by using routine sequence analysis tools as Basic Local Alignment Search Tool (BLAST) or CD-Search for conserved domain analysis. Molecular reconstructions can be created based upon sequence consensus, e.g. using approaches described in Ivics et al., Cell 1997, 501 510; Wagstaff et al., Molecular Biology and Evolution 2013, 88-99. In some embodiments, the retrotransposon from which the 5′ or 3′ untranslated region or polypeptide is derived is a young or a recently active mobile element, as assessed via phylogenetic methods such as those described in Boissinot et al., Molecular Biology and Evolution 2000, 915-928.

### Linkers

In some embodiments, domains of the compositions and systems described herein (e.g., the endonuclease and reverse transcriptase domains of a polypeptide or the DNA binding domain and reverse transcriptase domains of a polypeptide) may be joined by a linker. A composition described herein comprising a linker element has the general form S1-L-S2, wherein S1 and S2 may be the same or different and represent two domain moieties (e.g., each a polypeptide or nucleic acid domain) associated with one another by the linker. In some embodiments, a linker may connect two polypeptides. In some embodiments, a linker may connect two nucleic acid molecules. In some embodiments, a linker may connect a polypeptide and a nucleic acid molecule. A linker may be a chemical bond, e.g., one or more covalent bonds or non-covalent bonds. A linker may be flexible, rigid, and/or cleavable. In some embodiments, the linker is a peptide linker. Generally, a peptide linker is at least 2, 3, 4, 5, 6, 7, 8, 9, 10 or more amino acids in length, e.g., 2-50 amino acids in length, 2-30 amino acids in length.

Some commonly used flexible linkers have sequences consisting primarily of stretches of Gly and Ser residues (“GS” linker). Flexible linkers may be useful for joining domains that require a certain degree of movement or interaction and may include small, non-polar (e.g. Gly) or polar (e.g. Ser or Thr) amino acids. Incorporation of Ser or Thr can also maintain the stability of the linker in aqueous solutions by forming hydrogen bonds with the water molecules, and therefore reduce unfavorable interactions between the linker and the other moieties. Examples of such linkers include those having the structure [GGS]^{≥1} or [GGGS]^{≥1} (SEQ ID NO: 1536). Rigid linkers are useful to keep a fixed distance between domains and to maintain their independent functions. Rigid linkers may also be useful when a spatial separation of the domains is critical to preserve the stability or bioactivity of one or more components in the agent. Rigid linkers may have an alpha helix-structure or Pro-rich sequence, (XP)n, with X designating any amino acid, preferably Ala, Lys, or Glu. Cleavable linkers may release free functional domains in vivo. In some embodiments, linkers may be cleaved under specific conditions, such as the presence of reducing reagents or proteases. In vivo cleavable linkers may utilize the reversible nature of a disulfide bond. One example includes a thrombin-sensitive sequence (e.g., PRS) between the two Cys residues. In vitro thrombin treatment of CPRSC (SEQ ID NO: 1537) results in the cleavage of the thrombin-sensitive sequence, while the reversible disulfide linkage remains intact. Such linkers are known and described, e.g., in Chen et al. 2013. Fusion Protein Linkers: Property, Design and Functionality. Adv Drug Deliv Rev. 65(10): 1357-1369. In vivo cleavage of linkers in compositions described herein may also be carried out by proteases that are expressed in vivo under pathological conditions (e.g. cancer or inflammation), in specific cells or tissues, or constrained within certain cellular compartments. The specificity of many proteases offers slower cleavage of the linker in constrained compartments.

In some embodiments the amino acid linkers are (or are homologous to) the endogenous amino acids that exist between such domains in a native polypeptide. In some embodiments, the endogenous amino acids that exist between such domains are substituted but the length is unchanged from the natural length. In some embodiments, additional amino acid residues are added to the naturally existing amino acid residues between domains.

In some embodiments, the amino acid linkers are designed computationally or screened to maximize protein function (Anad et al., FEBS Letters, 587:19, 2013).

In addition to being fully encoded on a single transcript, a polypeptide can be generated by separately expressing two or more polypeptide fragments that reconstitute the holoenzyme. In some embodiments, the gene modifying polypeptide is generated by expressing as separate subunits that reassemble the holoenzyme through engineered protein-protein interactions. In some embodiments, reconstitution of the holoenzyme does not involve covalent binding between subunits. Peptides may also fuse together through trans-splicing of inteins (Tornabene et al. Sci Transl Med 11, eaav4523 (2019)). In some embodiments, the gene modifying holoenzyme is expressed as separate subunits that are designed to create a fusion protein through the presence of split inteins (e.g., as described herein) in the subunits. In some embodiments, the gene modifying holoenzyme is reconstituted through the formation of covalent linkages between subunits. In some embodiments, the breaking up of a gene modifying polypeptide into subunits may aid in delivery of the protein by keeping the nucleic acid encoding each part within optimal packaging limits of a viral delivery vector, e.g., AAV (Tornabene et al. Sci Transl Med 11, eaav4523 (2019)). In some embodiments, the gene modifying polypeptide is designed to be dimerized through the use of covalent or non-covalent interactions as described above. Exemplary Linkers are shown in Table L1 below.

TABLE L1
Exemplary linker sequences

| | SEQ |
|---|---|
| | ID |
| Amino Acid Sequence | NO |
| GGS | |
| GGSGGS | 102 |
| GGSGGSGGS | 103 |
| GGSGGSGGSGGS | 104 |
| GGSGGSGGSGGSGGS | 105 |
| GGSGGSGGSGGSGGSGGS | 106 |
| GGGGS | 107 |
| GGGGSGGGGS | 108 |
| GGGGSGGGGSGGGGS | 109 |
| GGGGSGGGGGGGGSGGGGS | 110 |
| GGGGSGGGGSGGGGSGGGGSGGGGS | 111 |
| GGGGSGGGGSGGGGSGGGGSGGGGSGGGGS | 112 |
| GGG | |
| GGGG | 114 |
| GGGGG | 115 |
| GGGGGG | 116 |
| GGGGGGG | 117 |
| GGGGGGGG | 118 |
| GSS | |
| GSSGSS | 120 |
| GSSGSSGSS | 121 |
| GSSGSSGSSGSS | 122 |
| GSSGSSGSSGSSGSS | 123 |
| GSSGSSGSSGSSGSSGSS | 124 |
| EAAAK | 125 |
| EAAAKEAAAK | 126 |
| EAAAKEAAAKEAAAK | 127 |
| EAAAKEAAAKEAAAKEAAAK | 128 |
| EAAAKEAAAKEAAAKEAAAKEAAAK | 129 |
| EAAAKEAAAKEAAAKEAAAKEAAAKEAAAK | 130 |
| PAP | |
| PAPAP | 132 |
| PAPAPAP | 133 |
| PAPAPAPAP | 134 |
| PAPAPAPAPAP | 135 |
| PAPAPAPAPAPAP | 136 |
| GGSGGG | 137 |
| GGGGGS | 138 |
| GGSGSS | 139 |
| GSSGGS | 140 |
| GGSEAAAK | 141 |
| EAAAKGGS | 142 |
| GGSPAP | 143 |
| PAPGGS | 144 |
| GGGGSS | 145 |
| GSSGGG | 146 |
| GGGEAAAK | 147 |
| EAAAKGGG | 148 |
| GGGPAP | 149 |
| PAPGGG | 150 |
| GSSEAAAK | 151 |
| EAAAKGSS | 152 |
| GSSPAP | 153 |
| PAPGSS | 154 |
| EAAAKPAP | 155 |
| PAPEAAAK | 156 |
| GGSGGGGSS | 157 |
| GGSGSSGGG | 158 |
| GGGGGSGSS | 159 |
| GGGGSSGGS | 160 |
| GSSGGSGGG | 161 |
| GSSGGGGGS | 162 |
| GGSGGGEAAAK | 163 |
| GGSEAAAKGGG | 164 |
| GGGGGSEAAAK | 165 |
| GGGEAAAKGGS | 166 |
| EAAAKGGSGGG | 167 |
| EAAAKGGGGGS | 168 |
| GGSGGGPAP | 169 |
| GGSPAPGGG | 170 |
| GGGGGSPAP | 171 |
| GGGPAPGGS | 172 |
| PAPGGSGGG | 173 |
| PAPGGGGGS | 174 |
| GGSGSSEAAAK | 175 |
| GGSEAAAKGSS | 176 |
| GSSGGSEAAAK | 177 |
| GSSEAAAKGGS | 178 |
| EAAAKGGSGSS | 179 |
| EAAAKGSSGGS | 180 |
| GGSGSSPAP | 181 |
| GGSPAPGSS | 182 |
| GSSGGSPAP | 183 |
| GSSPAPGGS | 184 |
| PAPGGSGSS | 185 |
| PAPGSSGGS | 186 |
| GGSEAAAKPAP | 187 |
| GGSPAPEAAAK | 188 |
| EAAAKGGSPAP | 189 |
| EAAAKPAPGGS | 190 |
| PAPGGSEAAAK | 191 |
| PAPEAAAKGGS | 192 |
| GGGGSSEAAAK | 193 |
| GGGEAAAKGSS | 194 |
| GSSGGGEAAAK | 195 |
| GSSEAAAKGGG | 196 |
| EAAAKGGGGSS | 197 |
| EAAAKGSSGGG | 198 |
| GGGGSSPAP | 199 |
| GGGPAPGSS | 200 |
| GSSGGGPAP | 201 |
| GSSPAPGGG | 202 |
| PAPGGGGSS | 203 |
| PAPGSSGGG | 204 |
| GGGEAAAKPAP | 205 |
| GGGPAPEAAAK | 206 |
| EAAAKGGGPAP | 207 |
| EAAAKPAPGGG | 208 |
| PAPGGGEAAAK | 209 |
| PAPEAAAKGGG | 210 |
| GSSEAAAKPAP | 211 |
| GSSPAPEAAAK | 212 |
| EAAAKGSSPAP | 213 |
| EAAAKPAPGSS | 214 |
| PAPGSSEAAAK | 215 |
| PAPEAAAKGSS | 216 |
| AEAAAKEAAAKEAAAKEAAAKALEAEAAAKEAAAKEAAAK | 217 |
| EAAAKA | |
| GGGGSEAAAKGGGGS | 218 |
| EAAAKGGGGSEAAAK | 219 |
| SGSETPGTSESATPES | 220 |
| GSAGSAAGSGEF | 221 |
| SGGSSGGSSGSETPGTSESATPESSGGSSGGSS | 222 |
| GSTSGSGKPGSGEGSTKG | 15520 |

In some embodiments, a linker of a gene modifying polypeptide comprises a motif chosen from: (SGGS)ₙ (SEQ ID NO: 25), (GGGS)ₙ (SEQ ID NO: 26), (GGGGS)ₙ (SEQ ID NO: 27), (G)ₙ, (EAAAK)ₙ (SEQ ID NO: 28), (GGS)ₙ, or (XP)ₙ.

### Inteins

In some embodiments, the gene modifying system comprises an intein. Generally, an intein comprises a polypeptide that has the capacity to join two polypeptides or polypeptide fragments together via a peptide bond. In some embodiments, the intein is a trans-splicing intein that can join two polypeptide fragments, e.g., to form the polypeptide component of a system as described herein. In some embodiments, an intein may be encoded on the same nucleic acid molecule encoding the two polypeptide fragments. In certain embodiments, the intein may be translated as part of a larger polypeptide comprising, e.g., in order, the first polypeptide fragment, the intein, and the second polypeptide fragment. In embodiments, the translated intein may be capable of excising itself from the larger polypeptide, e.g., resulting in separation of the attached polypeptide fragments. In embodiments, the excised intein may be capable of joining the two polypeptide fragments to each other directly via a peptide bond. In some embodiments, as described in more detail below, Intein-N may be fused to the N-terminal portion of a first domain described herein, and and intein-C may be fused to the C-terminal portion of a second domain described herein for the joining of the N-terminal portion to the C-terminal portion, thereby joining the first and second domains. In some embodiments, the first and second domains are each independent chosen from a DNA binding domain, an RNA binding domain, an RT domain, and an endonuclease domain.

As used herein, “intein” refers to a self-splicing protein intron (e.g., peptide), e.g., which ligates flanking N-terminal and C-terminal exteins (e.g., fragments to be joined). An intein may, in some instances, comprise a fragment of a protein that is able to excise itself and join the remaining fragments (the exteins) with a peptide bond in a process known as protein splicing. Inteins are also referred to as “protein introns.” The process of an intein excising itself and joining the remaining portions of the protein is herein termed “protein splicing” or “intein-mediated protein splicing.” In some embodiments, an intein of a precursor protein (an intein containing protein prior to intein-mediated protein splicing) comes from two genes. Such intein is referred to herein as a split intein (e.g., split intein-N and split intein-C). For example, in cyanobacteria, DnaE, the catalytic subunit a of DNA polymerase III, is encoded by two separate genes, dnaE-n and dnaE-c. The intein encoded by the dnaE-n gene may be herein referred as “intein-N.” The intein encoded by the dnaE-c gene may be herein referred as “intein-C.”

Use of inteins for joining heterologous protein fragments is described, for example, in Wood et al., J. Biol. Chem. 289(21); 14512-9 (2014) (incorporated herein by reference in its entirety). For example, when fused to separate protein fragments, the inteins IntN and IntC may recognize each other, splice themselves out, and/or simultaneously ligate the flanking N- and C-terminal exteins of the protein fragments to which they were fused, thereby reconstituting a full-length protein from the two protein fragments.

In some embodiments, a synthetic intein based on the dnaE intein, the Cfa-N (e.g., split intein-N) and Cfa-C (e.g., split intein-C) intein pair, is used. Examples of such inteins have been described, e.g., in Stevens et al., J Am Chem Soc. 2016 Feb. 24; 138(7):2162-5 (incorporated herein by reference in its entirety). Non-limiting examples of intein pairs that may be used in accordance with the present disclosure include: Cfa DnaE intein, Ssp GyrB intein, Ssp DnaX intein, Ter DnaE3 intein, Ter ThyX intein, Rma DnaB intein and Cne Prp8 intein (e.g., as described in U.S. Pat. No. 8,394,604, incorporated herein by reference.

In some embodiments, a protein fragment ranges from about 2-1000 amino acids (e.g., between 2-10, 10-50, 50-100, 100-200, 200-300, 300-400, 400-500, 500-600, 600-700, 700-800, 800-900, or 900-1000 amino acids) in length. In some embodiments, a protein fragment ranges from about 5-500 amino acids (e.g., between 5-10, 10-50, 50-100, 100-200, 200-300, 300-400, or 400-500 amino acids) in length. In some embodiments, a protein fragment ranges from about 20-200 amino acids (e.g., between 20-30, 30-40, 40-50, 50-100, or 100-200 amino acids) in length.

In some embodiments, a portion or fragment of a gene modifying polypeptide is fused to an intein. The nuclease can be fused to the N-terminus or the C-terminus of the intein. In some embodiments, a portion or fragment of a fusion protein is fused to an intein and fused to an AAV capsid protein. The intein, nuclease and capsid protein can be fused together in any arrangement (e.g., nuclease-intein-capsid, intein-nuclease-capsid, capsid-intein-nuclease, etc.). In some embodiments, the N-terminus of an intein is fused to the C-terminus of a fusion protein and the C-terminus of the intein is fused to the N-terminus of an AAV capsid protein.

In some embodiments, an endonuclease domain is fused to intein-N and a polypeptide comprising an RT domain is fused to an intein-C.

Exemplary nucleotide and amino acid sequences of interns are provided below:

DnaE Intein-N DNA:
(SEQ ID NO: 15412)
TGCCTGTCATACGAAACCGAGATACTGACAGTAGAATATGGCCTTCTGCCAATCGGG
AAGATTGTGGAGAAACGGATAGAATGCACAGTTTACTCTGTCGATAACAATGGTAA
CATTTATACTCAGCCAGTTGCCCAGTGGCACGACCGGGGAGAGCAGGAAGTATTCG
AATACTGTCTGGAGGATGGAAGTCTCATTAGGGCCACTAAGGACCACAAATTTATG
ACAGTCGATGGCCAGATGCTGCCTATAGACGAAATCTTTGAGCGAGAGTTGGACCTC
ATGCGAGTTGACAACCTTCCTAAT
DnaE Intein-N Protein:
(SEQ ID NO: 15413)
CLSYETEILTVEYGLLPIGKIVEKRIECTVYSVDNNGNIYTQPVAQWHDRGEQEVFEYCL
EDGSLIRATKDHKFMTVDGQMLPIDEIFERELDLMRVDNLPN
DnaE Intein-C DNA:
(SEQ ID NO: 15414)
ATGATCAAGATAGCTACAAGGAAGTATCTTGGCAAACAAAACGTTTATGATATTGG
AGTCGAAAGAGATCACAACTTTGCTCTGAAGAACGGATTCATAGCTTCTAAT
Intein-C:
(SEQ ID NO: 15415)
MIKIATRKYLGKQNVYDIGVERDHNFALKNGFIASN
Cfa-N DNA:
(SEQ ID NO: 15416)
TGCCTGTCTTATGATACCGAGATACTTACCGTTGAATATGGCTTCTTGCCTATTGGAA
AGATTGTCGAAGAGAGAATTGAATGCACAGTATATACTGTAGACAAGAATGGTTTC
GTTTACACACAGCCCATTGCTCAATGGCACAATCGCGGCGAACAAGAAGTATTTGA
GTACTGTCTCGAGGATGGAAGCATCATACGAGCAACTAAAGATCATAAATTCATGA
CCACTGACGGGCAGATGTTGCCAATAGATGAGATATTCGAGCGGGGCTTGGATCTC
AAACAAGTGGATGGATTG CCA
Cfa-N Protein:
(SEQ ID NO: 15417)
CLSYDTEILTVEYGFLPIGKIVEERIECTVYTVDKNGFVYTQPIAQWHNRGEQEVFEYCL
EDGSIIRATKDHKFMTTDGQMLPIDEIFERGLDLKQVDGLP
Cfa-C DNA:
(SEQ ID NO: 15418)
ATGAAGAGGACTGCCGATGGATCAGAGTTTGAATCTCCCAAGAAGAAGAGGAAAGT
AAAGATAATATCTCGAAAAAGTCTTGGTACCCAAAATGTCTATGATATTGGAGTGGA
GAAAGATCACAACTTCCTTCTCAAGAACGGTCTCGTAGCCAGCAAC
Cfa-C Protein:
(SEQ ID NO: 15419)
MKRTADGSEFESPKKKRKVKIISRKSLGTQNVYDIGVEKDHNFLLKNGLVASN

### Promoters

In some embodiments, one or more promoter or enhancer elements are operably linked to a nucleic acid encoding a gene modifying protein or a template nucleic acid, e.g., that controls expression of the heterologous object sequence. In certain embodiments, the one or more promoter or enhancer elements comprise cell-type or tissue specific elements. In some embodiments, the promoter or enhancer is the same or derived from the promoter or enhancer that naturally controls expression of the heterologous object sequence. For example, the ornithine transcarbomylase promoter and enhancer may be used to control expression of the ornithine transcarbomylase gene in a system or method provided by the invention for correcting ornithine transcarbomylase deficiencies. In some embodiments, a promoter for use in the invention is for a gene described in Table 33 or 34, e.g., which may be used with an allele of the reference gene, or, in other embodiments, with a heterologous gene. In some embodiments, the promoter is a promoter of Table 33 or a functional fragment or variant thereof.

Exemplary tissue specific promoters that are commercially available can be found, for example, at a uniform resource locator (e.g., invivogen.com/tissue-specific-promoters). In some embodiments, a promoter is a native promoter or a minimal promoter, e.g., which consists of a single fragment from the 5′ region of a given gene. In some embodiments, a native promoter comprises a core promoter and its natural 5′ UTR. In some embodiments, the 5′ UTR comprises an intron. In other embodiments, these include composite promoters, which combine promoter elements of different origins or were generated by assembling a distal enhancer with a minimal promoter of the same origin.

Exemplary cell or tissue specific promoters are provided in the tables, below, and exemplary nucleic acid sequences encoding them are known in the art and can be readily accessed using a variety of resources, such as the NCBI database, including RefSeq, as well as the Eukaryotic Promoter Database (epd.epfl.ch//index.php).

TABLE 33
Exemplary cell or tissue-specific promoters

| Promoter | Target cells |
|---|---|
| B29 Promoter | B cells |
| CD14 Promoter | Monocytic Cells |
| CD43 Promoter | Leukocytes and platelets |
| CD45 Promoter | Hematopoeitic cells |
| CD68 promoter | macrophages |
| Desmin promoter | muscle cells |
| Elastase-1 | pancreatic acinar cells |
| promoter | |
| Endoglin promoter | endothelial cells |
| fibronectin | differentiating cells, healing |
| promoter | tissue |
| Flt-1 promoter | endothelial cells |
| GFAP promoter | Astrocytes |
| GPIIB promoter | megakaryocytes |
| ICAM-2 Promoter | Endothelial cells |
| INF-Beta promoter | Hematopoeitic cells |
| Mb promoter | muscle cells |
| Nphs1 promoter | podocytes |
| OG-2 promoter | Osteoblasts, Odonblasts |
| SP-B promoter | Lung |
| Syn1 promoter | Neurons |
| WASP promoter | Hematopoeitic cells |
| SV40/bAlb | Liver |
| promoter | |
| SV40/bAlb | Liver |
| promoter | |
| SV40/Cd3 | Leukocytes and platelets |
| promoter | |
| SV40/CD45 | hematopoeitic cells |
| promoter | |
| NSE/RU5′ | Mature Neurons |
| promoter | |

TABLE 34
Additional exemplary cell or tissue-specific promoters

| Promoter | Gene Description | Gene Specificity |
|---|---|---|
| APOA2 | Apolipoprotein A-II | Hepatocytes (from hepatocyte |
| | | progenitors) |
| SERPINA | Serpin peptidase inhibitor, clade A | Hepatocytes |
| 1 (hAAT) | (alpha-1 | (from definitive endoderm |
| | antiproteinase, antitrypsin), member 1 | stage) |
| | (also named alpha 1 anti-tryps in) | |
| CYP3A | Cytochrome P450, family 3, | Mature Hepatocytes |
| | subfamily A, polypeptide | |
| MIR122 | MicroRNA 122 | Hepatocytes |
| | | (from early stage embryonic |
| | | liver cells) |
| | | and endoderm |

Pancreatic specific promoters

| | | |
|---|---|---|
| INS | Insulin | Pancreatic beta cells |
| | | (from definitive endoderm stage) |
| IRS2 | Insulin receptor substrate 2 | Pancreatic beta cells |
| Pdx1 | Pancreatic and duodenal | Pancreas |
| | homeobox 1 | (from definitive endoderm stage) |
| Alx3 | Aristaless-like homeobox 3 | Pancreatic beta cells |
| | | (from definitive endoderm stage) |
| Ppy | Pancreatic polypeptide | PP pancreatic cells |
| | | (gamma cells) |

Cardiac specific promoters

| | | |
|---|---|---|
| Myh6 | Myosin, heavy chain 6, cardiac | Late differentiation marker of cardiac |
| (aMHC) | muscle, alpha | muscle cells (atrial specificity) |
| MYL2 | Myosin, light chain 2, regulatory, | Late differentiation marker of cardiac |
| (MLC-2v) | cardiac, slow | muscle cells (ventricular specificity) |
| ITNNl3 | Troponin I type 3 (cardiac) | Cardiomyocytes |
| (cTnl) | | (from immature state) |
| ITNNl3 | Troponin I type 3 (cardiac) | Cardiomyocytes |
| (cTnl) | | (from immature state) |
| NPPA | Natriuretic peptide precursor A (also | Atrial specificity in adult cells |
| (ANF) | named Atrial Natriuretic Factor) | |
| Slc8a1 | Solute carrier family 8 | Cardiomyocytes from early |
| (Ncx1) | (sodium/calcium exchanger), member | developmental stages |
| | 1 | |

CNS specific promoters

| | | |
|---|---|---|
| SYN1 | Synapsin I | Neurons |
| (hSyn) | | |
| GFAP | Glial fibrillary acidic protein | Astrocytes |
| INA | Internexin neuronal intermediate | Neuroprogenitors |
| | filament protein, alpha (a-internexin) | |
| NES | Nestin | Neuroprogenitors and ectoderm |
| MOBP | Myelin-associated oligodendrocyte | Oligodendrocytes |
| | basic protein | |
| MBP | Myelin basic protein | Oligodendrocytes |
| TH | Tyrosine hydroxylase | Dopaminergic neurons |
| FOXA2 | Forkhead box A2 | Dopaminergic neurons (also used as a |
| (HNF3 | | marker of endoderm) |
| beta) | | |

Skin specific promoters

| | | |
|---|---|---|
| FLG | Filaggrin | Keratinocytes from granular layer |
| K14 | Keratin 14 | Keratinocytes from granular |
| | | and basal layers |
| TGM3 | Transglutaminase 3 | Keratinocytes from granular layer |

Immune cell specific promoters

| | | |
|---|---|---|
| ITGAM | Integrin, alpha M (complement | Monocytes, macrophages, granulocytes, |
| (CD11B) | component 3 receptor 3 subunit) | natural killer cells |

Urogential cell specific promoters

| | | |
|---|---|---|
| Pbsn | Probasin | Prostatic epithelium |
| Upk2 | Uroplakin 2 | Bladder |
| Sbp | Spermine binding protein | Prostate |
| Fer1l4 | Fer-1-like 4 | Bladder |

Endothelial cell specific promoters

| | | |
|---|---|---|
| ENG | Endoglin | Endothelial cells |

Pluripotent and embryonic cell specific promoters

| | | |
|---|---|---|
| Oct4 | POU class 5 homeobox 1 | Pluripotent cells |
| (POU5F1) | | (germ cells, ES cells, iPS cells) |
| NANOG | Nanog homeobox | Pluripotent cells |
| | | (ES cells, iPS cells) |
| Synthetic | Synthetic promoter based on a Oct-4 | Pluripotent cells (ES cells, iPS cells) |
| Oct4 | core enhancer element | |
| T | Brachyury | Mesoderm |
| brachyury | | |
| NES | Nestin | Neuroprogenitors and Ectoderm |
| SOX17 | SRY (sex determining region Y)-box | Endoderm |
| | 17 | |
| FOXA2 | Forkhead box A2 | Endoderm (also used as a marker of |
| (HNFJ | | dopaminergic neurons) |
| beta) | | |
| MIR122 | MicroRNA 122 | Endoderm and hepatocytes |
| | | (from early stage embryonic liver cells~ |

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544; incorporated herein by reference in its entirety).

In some embodiments, a nucleic acid encoding a gene modifying polypeptide or template nucleic acid is operably linked to a control element, e.g., a transcriptional control element, such as a promoter. The transcriptional control element may, in some embodiments, be functional in either a eukaryotic cell, e.g., a mammalian cell; or a prokaryotic cell (e.g., bacterial or archaeal cell). In some embodiments, a nucleotide sequence encoding a polypeptide is operably linked to multiple control elements, e.g., that allow expression of the nucleotide sequence encoding the polypeptide in both prokaryotic and eukaryotic cells.

For illustration purposes, examples of spatially restricted promoters include, but are not limited to, neuron-specific promoters, adipocyte-specific promoters, cardiomyocyte-specific promoters, smooth muscle-specific promoters, photoreceptor-specific promoters, etc. Neuron-specific spatially restricted promoters include, but are not limited to, a neuron-specific enolase (NSE) promoter (see, e.g., EMBL HSENO2, X51956); an aromatic amino acid decarboxylase (AADC) promoter, a neurofilament promoter (see, e.g., GenBank HUMNFL, L04147); a synapsin promoter (see, e.g., GenBank HUMSYNIB, M55301); a thy-1 promoter (see, e.g., Chen et al. (1987) Cell 51:7-19; and Llewellyn, et al. (2010) Nat. Med. 16(10):1161-1166); a serotonin receptor promoter (see, e.g., GenBank S62283); a tyrosine hydroxylase promoter (TH) (see, e.g., Oh et al. (2009) Gene Ther 16:437; Sasaoka et al. (1992) Mol. Brain Res. 16:274; Boundy et al. (1998) J. Neurosci. 18:9989; and Kaneda et al. (1991) Neuron 6:583-594); a GnRH promoter (see, e.g., Radovick et al. (1991) Proc. Natl. Acad. Sci. USA 88:3402-3406); an L7 promoter (see, e.g., Oberdick et al. (1990) Science 248:223-226); a DNMT promoter (see, e.g., Bartge et al. (1988) Proc. Natl. Acad. Sci. USA 85:3648-3652); an enkephalin promoter (see, e.g., Comb et al. (1988) EMBO J. 17:3793-3805); a myelin basic protein (MBP) promoter; a Ca2+-calmodulin-dependent protein kinase 11-alpha (CamKIIα) promoter (see, e.g., Mayford et al. (1996) Proc. Natl. Acad. Sci. USA 93:13250; and Casanova et al. (2001) Genesis 31:37); a CMV enhancer/platelet-derived growth factor-β promoter (see, e.g., Liu et al. (2004) Gene Therapy 11:52-60); and the like.

Adipocyte-specific spatially restricted promoters include, but are not limited to, the aP2 gene promoter/enhancer, e.g., a region from −5.4 kb to +21 bp of a human aP2 gene (see, e.g., Tozzo et al. (1997) Endocrinol. 138:1604; Ross et al. (1990) Proc. Natl. Acad. Sci. USA 87:9590; and Pavjani et al. (2005) Nat. Med. 11:797); a glucose transporter-4 (GLUT4) promoter (see, e.g., Knight et al. (2003) Proc. Natl. Acad. Sci. USA 100:14725); a fatty acid translocase (FAT/CD36) promoter (see, e.g., Kuriki et al. (2002) Biol. Pharm. Bull. 25:1476; and Sato et al. (2002) J. Biol. Chem. 277:15703); a stearoyl-CoA desaturase-1 (SCD1) promoter (Tabor et al. (1999) J. Biol. Chem. 274:20603); a leptin promoter (see, e.g., Mason et al. (1998) Endocrinol. 139:1013; and Chen et al. (1999) Biochem. Biophys. Res. Comm. 262:187); an adiponectin promoter (see, e.g., Kita et al. (2005) Biochem. Biophys. Res. Comm. 331:484; and Chakrabarti (2010) Endocrinol. 151:2408); an adipsin promoter (see, e.g., Platt et al. (1989) Proc. Natl. Acad. Sci. USA 86:7490); a resistin promoter (see, e.g., Seo et al. (2003) Molec. Endocrinol. 17:1522); and the like.

Cardiomyocyte-specific spatially restricted promoters include, but are not limited to, control sequences derived from the following genes: myosin light chain-2, α-myosin heavy chain, AE3, cardiac troponin C, cardiac actin, and the like. Franz et al. (1997) Cardiovasc. Res. 35:560-566; Robbins et al. (1995) Ann. N.Y. Acad. Sci. 752:492-505; Linn et al. (1995) Circ. Res. 76:584-591; Parmacek et al. (1994) Mol. Cell. Biol. 14:1870-1885; Hunter et al. (1993) Hypertension 22:608-617; and Sartorelli et al. (1992) Proc. Natl. Acad. Sci. USA 89:4047-4051.

Smooth muscle-specific spatially restricted promoters include, but are not limited to, an SM22α promoter (see, e.g., Akyurek et al. (2000) Mol. Med. 6:983; and U.S. Pat. No. 7,169,874); a smoothelin promoter (see, e.g., WO 2001/018048); an α-smooth muscle actin promoter; and the like. For example, a 0.4 kb region of the SM22α promoter, within which lie two CArG elements, has been shown to mediate vascular smooth muscle cell-specific expression (see, e.g., Kim, et al. (1997) Mol. Cell. Biol. 17, 2266-2278; Li, et al., (1996) J. Cell Biol. 132, 849-859; and Moessler, et al. (1996) Development 122, 2415-2425).

Photoreceptor-specific spatially restricted promoters include, but are not limited to, a rhodopsin promoter; a rhodopsin kinase promoter (Young et al. (2003) Ophthalmol. Vis. Sci. 44:4076); a beta phosphodiesterase gene promoter (Nicoud et al. (2007) J. Gene Med. 9:1015); a retinitis pigmentosa gene promoter (Nicoud et al. (2007) supra); an interphotoreceptor retinoid-binding protein (IRBP) gene enhancer (Nicoud et al. (2007) supra); an IRBP gene promoter (Yokoyama et al. (1992) Exp Eye Res. 55:225); and the like.

### Nonlimiting Exemplary Cell-Specific Promoters

Cell-specific promoters known in the art may be used to direct expression of a gene modifying protein, e.g., as described herein. Nonlimiting exemplary mammalian cell-specific promoters have been characterized and used in mice expressing Cre recombinase in a cell-specific manner. Certain nonlimiting exemplary mammalian cell-specific promoters are listed in Table 1 of U.S. Pat. No. 9,845,481, incorporated herein by reference.

In some embodiments, a cell-specific promoter is a promoter that is active in plants. Many exemplary cell-specific plant promoters are known in the art. See, e.g., U.S. Pat. Nos. 5,097,025; 5,783,393; 5,880,330; 5,981,727; 7,557,264; 6,291,666; 7,132,526; and 7,323,622; and U.S. Publication Nos. 2010/0269226; 2007/0180580; 2005/0034192; and 2005/0086712, which are incorporated by reference herein in their entireties for any purpose.

In some embodiments, a vector as described herein comprises an expression cassette. The term “expression cassette”, as used herein, refers to a nucleic acid construct comprising nucleic acid elements sufficient for the expression of the nucleic acid molecule of the instant invention. Typically, an expression cassette comprises the nucleic acid molecule of the instant invention operatively linked to a promoter sequence. The term“operatively linked” refers to the association of two or more nucleic acid fragments on a single nucleic acid fragment so that the function of one is affected by the other. For example, a promoter is operatively linked with a coding sequence when it is capable of affecting the expression of that coding sequence (e.g., the coding sequence is under the transcriptional control of the promoter). Encoding sequences can be operatively linked to regulatory sequences in sense or antisense orientation. In certain embodiments, the promoter is a heterologous promoter. The term“heterologous promoter”, as used herein, refers to a promoter that is not found to be operatively linked to a given encoding sequence in nature. In certain embodiments, an expression cassette may comprise additional elements, for example, an intron, an enhancer, a polyadenylation site, a woodchuck response element (WRE), and/or other elements known to affect expression levels of the encoding sequence A“promoter” typically controls the expression of a coding sequence or functional RNA. In certain embodiments, a promoter sequence comprises proximal and more distal upstream elements and can further comprise an enhancer element. An “enhancer” can typically stimulate promoter activity and may be an innate element of the promoter or a heterologous element inserted to enhance the level or tissue-specificity of a promoter. In certain embodiments, the promoter is derived in its entirety from a native gene. In certain embodiments, the promoter is composed of different elements derived from different naturally occurring promoters. In certain embodiments, the promoter comprises a synthetic nucleotide sequence. It will be understood by those skilled in the art that different promoters will direct the expression of a gene in different tissues or cell types, or at different stages of development, or in response to different environmental conditions or to the presence or the absence of a drug or transcriptional co-factor. Ubiquitous, cell-type-specific, tissue-specific, developmental stage-specific, and conditional promoters, for example, drug-responsive promoters (e.g., tetracycline-responsive promoters) are well known to those of skill in the art. Examples of promoter include, but are not limited to, the phosphoglycerate kinase (PKG) promoter, CAG (composite of the CMV enhancer the chicken beta actin promoter (CBA) and the rabbit beta globin intron.), NSE (neuronal specific enolase), synapsin or NeuN promoters, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), SFFV promoter, rous sarcoma virus (RSV) promoter, synthetic promoters, hybrid promoters, and the like. Other promoters can be of human origin or from other species, including from mice. Common promoters include, e.g., the human cytomegalovirus (CMV) immediate early gene promoter, the SV40 early promoter, the Rous sarcoma virus long terminal repeat, [beta]-actin, rat insulin promoter, the phosphoglycerate kinase promoter, the human alpha-1 antitrypsin (hAAT) promoter, the transthyretin promoter, the TBG promoter and other liver-specific promoters, the desmin promoter and similar muscle-specific promoters, the EF1-alpha promoter, the CAG promoter and other constitutive promoters, hybrid promoters with multi-tissue specificity, promoters specific for neurons like synapsin and glyceraldehyde-3-phosphate dehydrogenase promoter, all of which are promoters well known and readily available to those of skill in the art, can be used to obtain high-level expression of the coding sequence of interest. In addition, sequences derived from non-viral genes, such as the murine metallothionein gene, will also find use herein. Such promoter sequences are commercially available from, e.g., Stratagene (San Diego, CA). Additional exemplary promoter sequences are described, for example, in WO2018213786A1 (incorporated by reference herein in its entirety).

In some embodiments, the apolipoprotein E enhancer (ApoE) or a functional fragment thereof is used, e.g., to drive expression in the liver. In some embodiments, two copies of the ApoE enhancer or a functional fragment thereof is used. In some embodiments, the ApoE enhancer or functional fragment thereof is used in combination with a promoter, e.g., the human alpha-1 antitrypsin (hAAT) promoter.

In some embodiments, the regulatory sequences impart tissue-specific gene expression capabilities. In some cases, the tissue-specific regulatory sequences bind tissue-specific transcription factors that induce transcription in a tissue specific manner. Various tissue-specific regulatory sequences (e.g., promoters, enhancers, etc.) are known in the art. Exemplary tissue-specific regulatory sequences include, but are not limited to, the following tissue-specific promoters: a liver-specific thyroxin binding globulin (TBG) promoter, a insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), and others. Additional exemplary promoter sequences are described, for example, in U.S. patent Ser. No. 10/300,146 (incorporated herein by reference in its entirety). In some embodiments, a tissue-specific regulatory element, e.g., a tissue-specific promoter, is selected from one known to be operably linked to a gene that is highly expressed in a given tissue, e.g., as measured by RNA-seq or protein expression data, or a combination thereof. Methods for analyzing tissue specificity by expression are taught in Fagerberg et al. Mol Cell Proteomics 13(2):397-406 (2014), which is incorporated herein by reference in its entirety.

In some embodiments, a vector described herein is a multicistronic expression construct. Multicistronic expression constructs include, for example, constructs harboring a first expression cassette, e.g. comprising a first promoter and a first encoding nucleic acid sequence, and a second expression cassette, e.g. comprising a second promoter and a second encoding nucleic acid sequence. Such multicistronic expression constructs may, in some instances, be particularly useful in the delivery of non-translated gene products, such as hairpin RNAs, together with a polypeptide, for example, a gene modifying polypeptide and gene modifying template. In some embodiments, multicistronic expression constructs may exhibit reduced expression levels of one or more of the included transgenes, for example, because of promoter interference or the presence of incompatible nucleic acid elements in close proximity. If a multicistronic expression construct is part of a viral vector, the presence of a self-complementary nucleic acid sequence may, in some instances, interfere with the formation of structures necessary for viral reproduction or packaging.

In some embodiments, the sequence encodes an RNA with a hairpin. In some embodiments, the hairpin RNA is a guide RNA, a template RNA, shRNA, or a microRNA. In some embodiments, the first promoter is an RNA polymerase I promoter. In some embodiments, the first promoter is an RNA polymerase II promoter. In some embodiments, the second promoter is an RNA polymerase III promoter. In some embodiments, the second promoter is a U6 or H1 promoter. In some embodiments, the nucleic acid construct comprises the structure of AAV construct B1 or B2.

Without wishing to be bound by theory, multicistronic expression constructs may not achieve optimal expression levels as compared to expression systems containing only one cistron. One of the suggested causes of lower expression levels achieved with multicistronic expression constructs comprising two ore more promoter elements is the phenomenon of promoter interference (see, e.g., Curtin J A, Dane A P, Swanson A, Alexander I E, Ginn S L. Bidirectional promoter interference between two widely used internal heterologous promoters in a late-generation lentiviral construct. Gene Ther. 2008 March; 15(5):384-90; and Martin-Duque P, Jezzard S, Kaftansis L, Vassaux G. Direct comparison of the insulating properties of two genetic elements in an adenoviral vector containing two different expression cassettes. Hum Gene Ther. 2004 October; 15(10):995-1002; both references incorporated herein by reference for disclosure of promoter interference phenomenon). In some embodiments, the problem of promoter interference may be overcome, e.g., by producing multicistronic expression constructs comprising only one promoter driving transcription of multiple encoding nucleic acid sequences separated by internal ribosomal entry sites, or by separating cistrons comprising their own promoter with transcriptional insulator elements. In some embodiments, single-promoter driven expression of multiple cistrons may result in uneven expression levels of the cistrons. In some embodiments, a promoter cannot efficiently be isolated and isolation elements may not be compatible with some gene transfer vectors, for example, some retroviral vectors.

### MicroRNAs

miRNAs and other small interfering nucleic acids generally regulate gene expression via target RNA transcript cleavage/degradation or translational repression of the target messenger RNA (mRNA). miRNAs may, in some instances, be natively expressed, typically as final 19-25 non-translated RNA products. miRNAs generally exhibit their activity through sequence-specific interactions with the 3′ untranslated regions (UTR) of target mRNAs. These endogenously expressed miRNAs may form hairpin precursors that are subsequently processed into an miRNA duplex, and further into a mature single stranded miRNA molecule. This mature miRNA generally guides a multiprotein complex, miRISC, which identifies target 3′ UTR regions of target mRNAs based upon their complementarity to the mature miRNA. Useful transgene products may include, for example, miRNAs or miRNA binding sites that regulate the expression of a linked polypeptide. A non-limiting list of miRNA genes; the products of these genes and their homologues are useful as transgenes or as targets for small interfering nucleic acids (e.g., miRNA sponges, antisense oligonucleotides), e.g., in methods such as those listed in U.S. Ser. No. 10/300,146, 22:25-25:48, incorporated by reference. In some embodiments, one or more binding sites for one or more of the foregoing miRNAs are incorporated in a transgene, e.g., a transgene delivered by a rAAV vector, e.g., to inhibit the expression of the transgene in one or more tissues of an animal harboring the transgene. In some embodiments, a binding site may be selected to control the expression of a transgene in a tissue specific manner. For example, binding sites for the liver-specific miR-122 may be incorporated into a transgene to inhibit expression of that transgene in the liver. Additional exemplary miRNA sequences are described, for example, in U.S. patent Ser. No. 10/300,146 (incorporated herein by reference in its entirety). For liver-specific gene modifying, however, overexpression of miR-122 may be utilized instead of using binding sites to effect miR-122-specific degradation. This miRNA is positively associated with hepatic differentiation and maturation, as well as enhanced expression of liver specific genes. Thus, in some embodiments, the coding sequence for miR-122 may be added to a component of a gene modifying system to enhance a liver-directed therapy.

A miR inhibitor or miRNA inhibitor is generally an agent that blocks miRNA expression and/or processing. Examples of such agents include, but are not limited to, microRNA antagonists, microRNA specific antisense, microRNA sponges, and microRNA oligonucleotides (double-stranded, hairpin, short oligonucleotides) that inhibit miRNA interaction with a Drosha complex. MicroRNA inhibitors, e.g., miRNA sponges, can be expressed in cells from transgenes (e.g., as described in Ebert, M. S. Nature Methods, Epub Aug. 12, 2007; incorporated by reference herein in its entirety). In some embodiments, microRNA sponges, or other miR inhibitors, are used with the AAVs. microRNA sponges generally specifically inhibit miRNAs through a complementary heptameric seed sequence. In some embodiments, an entire family of miRNAs can be silenced using a single sponge sequence. Other methods for silencing miRNA function (derepression of miRNA targets) in cells will be apparent to one of ordinary skill in the art.

In some embodiments, a miRNA as described herein comprises a sequence listed in Table 4 of PCT Publication No. WO2020014209, incorporated herein by reference. Also incorporated herein by reference are the listing of exemplary miRNA sequences from WO2020014209.

In some embodiments, it is advantageous to silence one or more components of a gene modifying system (e.g., mRNA encoding a gene modifying polypeptide, a gene modifying Template RNA, or a heterologous object sequence expressed from the genome after successful gene modifying) in a portion of cells. In some embodiments, it is advantageous to restrict expression of a component of a gene modifying system to select cell types within a tissue of interest.

For example, it is known that in a given tissue, e.g., liver, macrophages and immune cells, e.g., Kupffer cells in the liver, may engage in uptake of a delivery vehicle for one or more components of a gene modifying system. In some embodiments, at least one binding site for at least one miRNA highly expressed in macrophages and immune cells, e.g., Kupffer cells, is included in at least one component of a gene modifying system, e.g., nucleic acid encoding a gene modifying polypeptide or a transgene. In some embodiments, a miRNA that targets the one or more binding sites is listed in a table referenced herein, e.g., miR-142, e.g., mature miRNA hsa-miR-142-5p or hsa-miR-142-3p.

In some embodiments, there may be a benefit to decreasing gene modifying polypeptide levels and/or gene modifying activity in cells in which gene modifying polypeptide expression or overexpression of a transgene may have a toxic effect. For example, it has been shown that delivery of a transgene overexpression cassette to dorsal root ganglion neurons may result in toxicity of a gene therapy (see Hordeaux et al Sci Transl Med 12(569):eaba9188 (2020), incorporated herein by reference in its entirety). In some embodiments, at least one miRNA binding site may be incorporated into a nucleic acid component of a gene modifying system to reduce expression of a system component in a neuron, e.g., a dorsal root ganglion neuron. In some embodiments, the at least one miRNA binding site incorporated into a nucleic acid component of a gene modifying system to reduce expression of a system component in a neuron is a binding site of miR-182, e.g., mature miRNA hsa-miR-182-5p or hsa-miR-182-3p. In some embodiments, the at least one miRNA binding site incorporated into a nucleic acid component of a gene modifying system to reduce expression of a system component in a neuron is a binding site of miR-183, e.g., mature miRNA hsa-miR-183-5p or hsa-miR-183-3p. In some embodiments, combinations of miRNA binding sites may be used to enhance the restriction of expression of one or more components of a gene modifying system to a tissue or cell type of interest.

Table A5 below provides exemplary miRNAs and corresponding expressing cells, e.g., a miRNA for which one can, in some embodiments, incorporate binding sites (complementary sequences) in the transgene or polypeptide nucleic acid, e.g., to decrease expression in that off-target cell.

TABLE A5
Exemplary miRNA from off-target cells and tissues

| Silenced | miRNA | Mature | | SEQ |
|---|---|---|---|---|
| cell type | name | miRNA | miRNA sequence | ID NO: |
| Kupffer cells | miR-142 | hsa-miR-142-5p | cauaaaguagaaagcacuacu | 15420 |
| Kupffer cells | miR-142 | hsa-miR-142-3p | uguaguguuuccuacuuuaugga | 15421 |
| Dorsal root | miR-182 | hsa-miR-182-5p | uuuggcaaugguagaacucacacu | 15422 |
| ganglion neurons | | | | |
| Dorsal root | miR-182 | hsa-miR-182-3p | ugguucuagacuugccaacua | 15423 |
| ganglion neurons | | | | |
| Dorsal root | miR-183 | hsa-miR-183-5p | uauggcacugguagaauucacu | 15424 |
| ganglion neurons | | | | |
| Table XDorsal | miR-183 | hsa-miR-183-3p | gugaauuaccgaagggccauaa | 15425 |
| root ganglion | | | | |
| neurons | | | | |
| Hepatocytes | miR-122 | hsa-miR-122-5p | uggagugugacaaugguguuug | 15426 |
| Hepatocytes | miR-122 | hsa-miR-122-3p | aacgccauuaucacacuaaaua | 15427 |

In some embodiments, a gene modifying system is capable of producing a substitution into the target site of at least 1, 2, 3, 4, 5, 6, 7, 8, 9, 10, 15, 20, 25, 30, 35, 40, 45, 50, 60, 70, 80, 90, or 100 or more nucleotides. In some embodiments, the substitution is a transition mutation. In some embodiments, the substitution is a transversion mutation. In some embodiments, the substitution converts an adenine to a thymine, an adenine to a guanine, an adenine to a cytosine, a guanine to a thymine, a guanine to a cytosine, a guanine to an adenine, a thymine to a cytosine, a thymine to an adenine, a thymine to a guanine, a cytosine to an adenine, a cytosine to a guanine, or a cytosine to a thymine.

In some embodiments, a gene modifying system is capable of producing an insertion into the target site of at least 45, 50, 55, 60, 65, 70, 75, 80, 85, 90, 95, or 100 nucleotides (and optionally no more than 500, 400, 300, 200, or 100 nucleotides). In some embodiments, a gene modifying system is capable of producing an insertion into the target site of at least 1, 2, 3, 4, 5, 6, 7, 8, 9, 10, 15, 20, 25, 30, 35, 40, 45, 50, 55, 60, 65, 70, 75, 80, 85, 90, 95, or 100 nucleotides (and optionally no more than 500, 400, 300, 200, or 100 nucleotides). In some embodiments, a gene modifying system is capable of producing an insertion into the target site of at least 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5 or 10 kilobases (and optionally no more than 1, 5, 10, or 20 kilobases). In some embodiments, a gene modifying system is capable of producing a deletion of at least 81, 85, 90, 95, 100, 110, 120, 130, 140, 150, 160, 170, 180, 190, or 200 nucleotides (and optionally no more than 500, 400, 300, or 200 nucleotides). In some embodiments, a gene modifying system is capable of producing a deletion of at least 81, 85, 90, 95, 100, 110, 120, 130, 140, 150, 160, 170, 180, 190, or 200 nucleotides (and optionally no more than 500, 400, 300, or 200 nucleotides). In some embodiments, a gene modifying system is capable of producing a deletion of at least 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200 nucleotides (and optionally no more than 500, 400, 300, or 200 nucleotides). In some embodiments, a gene modifying system is capable of producing a deletion of at least 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5 or 10 kilobases (and optionally no more than 1, 5, 10, or 20 kilobases).

In some embodiments, an insertion, deletion, substitution, or combination thereof, increases or decreases expression (e.g. transcription or translation) of a gene. In some embodiments, an insertion, deletion, substitution, or combination thereof, increases or decreases expression (e.g. transcription or translation) of a gene by altering, adding, or deleting sequences in a promoter or enhancer, e.g. sequences that bind transcription factors. In some embodiments, an insertion, deletion, substitution, or combination thereof alters translation of a gene (e.g. alters an amino acid sequence), inserts or deletes a start or stop codon, alters or fixes the translation frame of a gene. In some embodiments, an insertion, deletion, substitution, or combination thereof alters splicing of a gene, e.g. by inserting, deleting, or altering a splice acceptor or donor site. In some embodiments, an insertion, deletion, substitution, or combination thereof alters transcript or protein half-life. In some embodiments, an insertion, deletion, substitution, or combination thereof alters protein localization in the cell (e.g. from the cytoplasm to a mitochondria, from the cytoplasm into the extracellular space (e.g. adds a secretion tag)). In some embodiments, an insertion, deletion, substitution, or combination thereof alters (e.g. improves) protein folding (e.g. to prevent accumulation of misfolded proteins). In some embodiments, an insertion, deletion, substitution, or combination thereof, alters, increases, decreases the activity of a gene, e.g. a protein encoded by the gene.

In some embodiments, the gene modifying polypeptide results in insertion of the heterologous object sequence (e.g., the GFP gene) into the target locus (e.g., rDNA) at an average copy number of at least 0.01, 0.025, 0.05, 0.075, 0.1, 0.15, 0.2, 0.25, 0.3, 0.4, 0.5, 0.75, 1, 1.25, 1.5, 1.75, 2, 2.5, 3, 4, or 5 copies per genome. In some embodiments, a cell described herein (e.g., a cell comprising a heterologous sequence at a target insertion site) comprises the heterologous object sequence at an average copy number of at least 0.01, 0.025, 0.05, 0.075, 0.1, 0.15, 0.2, 0.25, 0.3, 0.4, 0.5, 0.75, 1, 1.25, 1.5, 1.75, 2, 2.5, 3, 4, or 5 copies per genome.

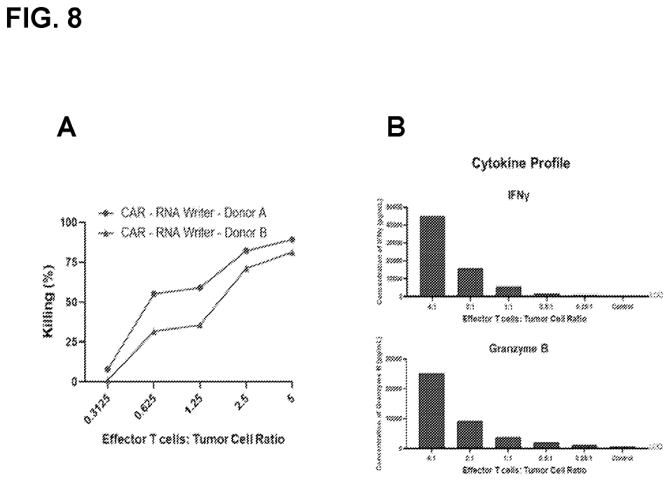

In some embodiments, a gene modifying system causes integration of a sequence in a target DNA with relatively few truncation events at the terminus. For instance, in some embodiments, a gene modifying protein results in about 25-100%, 50-100%, 60-100%, 70-100%, 75-95%, 80%-90%, or 86.17% of integrants into the target site being non-truncated, as measured by an assay described herein, e.g., an assay of Example 6 and FIG. 8 of PCT Application No. PCT/US2019/048607, incorporated herein by reference in its entirety. In some embodiments, a gene modifying protein results in at least about 30%, 40%, 50%, 60%, 70%, 80%, or 90% of integrants into the target site being non-truncated, as measured by an assay described herein. In some embodiments, an integrant is classified as truncated versus non-truncated using an assay comprising amplification with a forward primer situated 565 bp from the end of the element (e.g., a wild-type retrotransposon sequence) and a reverse primer situated in the genomic DNA of the target insertion site, e.g., rDNA. In some embodiments, the number of full-length integrants in the target insertion site is greater than the number of integrants truncated by 300-565 nucleotides in the target insertion site, e.g., the number of full-length integrants is at least 1.1×, 1.2×, 1.5×, 2×, 3×, 4×, 5×, 6×, 7×, 8×, 9×, or 10× the number of the truncated integrants, or the number of full-length integrants is at least 1.1×-10×, 2×-10×, 3×-10×, or 5×-10× the number of the truncated integrants.

In some embodiments, a system or method described herein results in insertion of the heterologous object sequence only at one target site in the genome of the target cell. Insertion can be measured, e.g., using a threshold of above 1%, 1.5%, 2%, 2.5%, 3%, 3.5%, 4%, 4.5%, 5%, e.g., as described in Example 8 of PCT Application No. PCT/US2019/048607, incorporated herein by reference in its entirety. In some embodiments, a system or method described herein results in insertion of the heterologous object sequence wherein less than 1%, 1.5%, 2%, 2.5%, 3%, 3.5%, 4%, 4.5%, 5%, 10%, 20%, 30%, 40%, or 50% of insertions are at a site other than the target site, e.g., using an assay described herein, e.g., an assay of Example 8 of PCT Application No. PCT/US2019/048607, incorporated herein by reference in its entirety.

In some embodiments, a system or method described herein results in “scarless” insertion of the heterologous object sequence, while in some embodiments, the target site can show deletions or duplications of endogenous DNA as a result of insertion of the heterologous sequence. The mechanisms of different retrotransposons could result in different patterns of duplications or deletions in the host genome occurring during retrotransposition at the target site. In some embodiments, the system results in a scarless insertion, with no duplications or deletions in the surrounding genomic DNA. In some embodiments, the system results in a deletion of less than 1, 2, 3, 4, 5, 10, 50, or 100 bp of genomic DNA upstream of the insertion. In some embodiments, the system results in a deletion of less than 1, 2, 3, 4, 5, 10, 50, or 100 bp of genomic DNA downstream of the insertion. In some embodiments, the system results in a duplication of less than 1, 2, 3, 4, 5, 10, 50, or 100 bp of genomic DNA upstream of the insertion. In some embodiments, the system results in a duplication of less than 1, 2, 3, 4, 5, 10, 50, or 100 bp of genomic DNA downstream of the insertion.

In some embodiments, a gene modifying system described herein, or a DNA-binding domain thereof, binds to its target site specifically, e.g., as measured using an assay of Example 21 of PCT Application No. PCT/US2019/048607, incorporated herein by reference in its entirety. In some embodiments, the gene modifying polypeptide or DNA-binding domain thereof binds to its target site more strongly than to any other binding site in the human genome. For example, in some embodiments, in an assay of Example 21 of PCT Application No. PCT/US2019/048607, incorporated herein by reference in its entirety, the target site represents more than 50%, 60%, 70%, 80%, 90%, or 95% of binding events of the gene modifying polypeptide or DNA-binding domain thereof to human genomic DNA. In some embodiments, the DNA binding domain of the gene modifying polypeptide is heterologous to the remainder of the gene modifying polypeptide, e.g., such that the gene modifying polypeptide targets a different target site that the endogenous DNA binding domain associated with the remainder of the gene modifying polypeptide.

### Genetically Engineered, e.g., Dimerized Gene Modifying Polypeptides

Some non-LTR retrotransposons utilize two subunits to complete retrotransposition (Christensen et al PNAS 2006). In some embodiments, a retrotransposase described herein comprises two connected subunits as a single polypeptide. For instance, two wild-type retrotransposases could be joined with a linker to form a covalently “dimerized” protein. In some embodiments, the nucleic acid coding for the retrotransposase codes for two retrotransposase subunits to be expressed as a single polypeptide. In some embodiments, the subunits are connected by a peptide linker, such as has been described herein in the section entitled “Linker” and, e.g., in Chen et al Adv Drug Deliv Rev 2013. In some embodiments, the two subunits in the polypeptide are connected by a rigid linker. In some embodiments, the rigid linker consists of the motif (EAAAK)ₙ (SEQ ID NO: 1534). In other embodiments, the two subunits in the polypeptide are connected by a flexible linker. In some embodiments, the flexible linker consists of the motif (Gly)ₙ. In some embodiments, the flexible linker consists of the motif (GGGGS)ₙ (SEQ ID NO: 1535). In some embodiments, the rigid or flexible linker consists of 1, 2, 3, 4, 5, 10, 15, or more amino acids in length to enable retrotransposition. In some embodiments, the linker consists of a combination of rigid and flexible linker motifs.

Based on mechanism, not all functions are required from both retrotransposase subunits. In some embodiments, the fusion protein may consist of a fully functional subunit and a second subunit lacking one or more functional domains. In some embodiments, one subunit may lack reverse transcriptase functionality. In some embodiments, one subunit may lack the reverse transcriptase domain. In some embodiments, one subunit may possess only endonuclease activity. In some embodiments, one subunit may possess only an endonuclease domain. In some embodiments, the two subunits comprising the single polypeptide may provide complimentary functions.

In some embodiments, one subunit may lack endonuclease functionality. In some embodiments, one subunit may lack the endonuclease domain. In some embodiments, one subunit may possess only reverse transcriptase activity. In some embodiments, one subunit may possess only a reverse transcriptase domain. In some embodiments, one subunit may possess only DNA-dependent DNA synthesis functionality.

### Evolved Variants of Gene Modifying Polypeptides

In some embodiments, the invention provides evolved variants of gene modifying polypeptides. Evolved variants can, in some embodiments, be produced by mutagenizing a reference gene modifying polypeptide, or one of the fragments or domains comprised therein. In some embodiments, one or more of the domains (e.g., the reverse transcriptase, DNA binding (including, for example, sequence-guided DNA binding elements), RNA-binding, or endonuclease domain) is evolved. One or more of such evolved variant domains can, in some embodiments, be evolved alone or together with other domains. An evolved variant domain or domains may, in some embodiments, be combined with unevolved cognate component(s) or evolved variants of the cognate component(s), e.g., which may have been evolved in either a parallel or serial manner.

In some embodiments, the process of mutagenizing a reference gene modifying polypeptide, or fragment or domain thereof, comprises mutagenizing the reference gene modifying polypeptide or fragment or domain thereof. In embodiments, the mutagenesis comprises a continuous evolution method (e.g., PACE) or non-continuous evolution method (e.g., PANCE), e.g., as described herein. In some embodiments, the evolved gene modifying polypeptide, or a fragment or domain thereof, comprises one or more amino acid variations introduced into its amino acid sequence relative to the amino acid sequence of the reference gene modifying polypeptide, or fragment or domain thereof. In embodiments, amino acid sequence variations may include one or more mutated residues (e.g., conservative substitutions, non-conservative substitutions, or a combination thereof) within the amino acid sequence of a reference gene modifying polypeptide, e.g., as a result of a change in the nucleotide sequence encoding the gene modifying polypeptide that results in, e.g., a change in the codon at any particular position in the coding sequence, the deletion of one or more amino acids (e.g., a truncated protein), the insertion of one or more amino acids, or any combination of the foregoing. The evolved variant gene modifying polypeptide may include variants in one or more components or domains of the gene modifying polypeptide (e.g., variants introduced into a reverse transcriptase domain, endonuclease domain, DNA binding domain, RNA binding domain, or combinations thereof).

In some aspects, the invention provides gene modifying polypeptides, systems, kits, and methods using or comprising an evolved variant of a gene modifying polypeptide, e.g., employs an evolved variant of a gene modifying polypeptide or a gene modifying polypeptide produced or producible by PACE or PANCE. In embodiments, the unevolved reference gene modifying polypeptide is a gene modifying polypeptide as disclosed herein.

The term “phage-assisted continuous evolution (PACE),” as used herein, generally refers to continuous evolution that employs phage as viral vectors. Examples of PACE technology have been described, for example, in International PCT Application No. PCT/US 2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Pat. No. 9,023,594, issued May 5, 2015; U.S. Pat. No. 9,771,574, issued Sep. 26, 2017; U.S. Pat. No. 9,394,537, issued Jul. 19, 2016; International PCT Application, PCT/US2015/012022, filed Jan. 20, 2015, published as WO 2015/134121 on Sep. 11, 2015; U.S. Pat. No. 10,179,911, issued Jan. 15, 2019; and International PCT Application, PCT/US2016/027795, filed Apr. 15, 2016, published as WO 2016/168631 on Oct. 20, 2016, the entire contents of each of which are incorporated herein by reference.

The term “phage-assisted non-continuous evolution (PANCE),” as used herein, generally refers to non-continuous evolution that employs phage as viral vectors. Examples of PANCE technology have been described, for example, in Suzuki T. et al, Crystal structures reveal an elusive functional domain of pyrrolysyl-tRNA synthetase, Nat Chem Biol. 13(12): 1261-1266 (2017), incorporated herein by reference in its entirety. Briefly, PANCE is a technique for rapid in vivo directed evolution using serial flask transfers of evolving selection phage (SP), which contain a gene of interest to be evolved, across fresh host cells (e.g., E. coli cells). Genes inside the host cell may be held constant while genes contained in the SP continuously evolve. Following phage growth, an aliquot of infected cells may be used to transfect a subsequent flask containing host E. coli. This process can be repeated and/or continued until the desired phenotype is evolved, e.g., for as many transfers as desired.

Methods of applying PACE and PANCE to gene modifying polypeptide may be readily appreciated by the skilled artisan by reference to, inter alia, the foregoing references. Additional exemplary methods for directing continuous evolution of genome-modifying proteins or systems, e.g., in a population of host cells, e.g., using phage particles, can be applied to generate evolved variants of gene modifying polypeptide, or fragments or subdomains thereof. Non-limiting examples of such methods are described in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Pat. No. 9,023,594, issued May 5, 2015; U.S. Pat. No. 9,771,574, issued Sep. 26, 2017; U.S. Pat. No. 9,394,537, issued Jul. 19, 2016; International PCT Application, PCT/US2015/012022, filed Jan. 20, 2015, published as WO 2015/134121 on Sep. 11, 2015; U.S. Pat. No. 10,179,911, issued Jan. 15, 2019; International Application No. PCT/US2019/37216, filed Jun. 14, 2019, International Patent Publication WO 2019/023680, published Jan. 31, 2019, International PCT Application, PCT/US2016/027795, filed Apr. 15, 2016, published as WO 2016/168631 on Oct. 20, 2016, and International Patent Publication No. PCT/US2019/47996, filed Aug. 23, 2019, each of which is incorporated herein by reference in its entirety.

In some non-limiting illustrative embodiments, a method of evolution of a evolved variant gene modifying polypeptide, of a fragment or domain thereof, comprises: (a) contacting a population of host cells with a population of viral vectors comprising the gene of interest (the starting gene modifying polypeptide or fragment or domain thereof), wherein: (1) the host cell is amenable to infection by the viral vector; (2) the host cell expresses viral genes required for the generation of viral particles; (3) the expression of at least one viral gene required for the production of an infectious viral particle is dependent on a function of the gene of interest; and/or (4) the viral vector allows for expression of the protein in the host cell, and can be replicated and packaged into a viral particle by the host cell. In some embodiments, the method comprises (b) contacting the host cells with a mutagen, using host cells with mutations that elevate mutation rate (e.g., either by carrying a mutation plasmid or some genome modification—e.g., proofing-impaired DNA polymerase, SOS genes, such as UmuC, UmuD′, and/or RecA, which mutations, if plasmid-bound, may be under control of an inducible promoter), or a combination thereof. In some embodiments, the method comprises (c) incubating the population of host cells under conditions allowing for viral replication and the production of viral particles, wherein host cells are removed from the host cell population, and fresh, uninfected host cells are introduced into the population of host cells, thus replenishing the population of host cells and creating a flow of host cells. In some embodiments, the cells are incubated under conditions allowing for the gene of interest to acquire a mutation. In some embodiments, the method further comprises (d) isolating a mutated version of the viral vector, encoding an evolved gene product (e.g., an evolved variant gene modifying polypeptide, or fragment or domain thereof), from the population of host cells.

The skilled artisan will appreciate a variety of features employable within the above-described framework. For example, in some embodiments, the viral vector or the phage is a filamentous phage, for example, an M13 phage, e.g., an M13 selection phage. In certain embodiments, the gene required for the production of infectious viral particles is the M13 gene III (gIII). In embodiments, the phage may lack a functional gIII, but otherwise comprise gI, gII, gIV, gV, gVI, gVII, gVIII, gIX, and a gX. In some embodiments, the generation of infectious VSV particles involves the envelope protein VSV-G. Various embodiments can use different retroviral vectors, for example, Murine Leukemia Virus vectors, or Lentiviral vectors. In embodiments, the retroviral vectors can efficiently be packaged with VSV-G envelope protein, e.g., as a substitute for the native envelope protein of the virus.

In some embodiments, host cells are incubated according to a suitable number of viral life cycles, e.g., at least 10, at least 20, at least 30, at least 40, at least 50, at least 100, at least 200, at least 300, at least 400, at least, 500, at least 600, at least 700, at least 800, at least 900, at least 1000, at least 1250, at least 1500, at least 1750, at least 2000, at least 2500, at least 3000, at least 4000, at least 5000, at least 7500, at least 10000, or more consecutive viral life cycles, which in on illustrative and non-limiting examples of M13 phage is 10-20 minutes per virus life cycle. Similarly, conditions can be modulated to adjust the time a host cell remains in a population of host cells, e.g., about 10, about 11, about 12, about 13, about 14, about 15, about 16, about 17, about 18, about 19, about 20, about 21, about 22, about 23, about 24, about 25, about 30, about 35, about 40, about 45, about 50, about 55, about 60, about 70, about 80, about 90, about 100, about 120, about 150, or about 180 minutes. Host cell populations can be controlled in part by density of the host cells, or, in some embodiments, the host cell density in an inflow, e.g., 10³ cells/ml, about 10⁴ cells/ml, about 10⁵ cells/ml, about 5-10⁵ cells/ml, about 10⁶ cells/ml, about 5-10⁶ cells/ml, about 10⁷ cells/ml, about 5-10⁷ cells/ml, about 10⁸ cells/ml, about 5-10⁸ cells/ml, about 10⁹ cells/ml, about 5·10⁹ cells/ml, about 10¹⁰ cells/ml, or about 5·10¹⁰ cells/ml.

### Template RNA Component of Gene Modifying System

The gene modifying systems described herein can transcribe an RNA sequence template into host target DNA sites by target-primed reverse transcription. By writing DNA sequence(s) via reverse transcription of the RNA sequence template directly into the host genome, the gene modifying system can insert an object sequence into a target genome without the need for exogenous DNA sequences to be introduced into the host cell (unlike, for example, CRISPR systems), as well as eliminate an exogenous DNA insertion step. Therefore, the gene modifying system provides a platform for the use of customized RNA sequence templates containing object sequences, e.g., sequences comprising heterologous gene coding and/or function information.

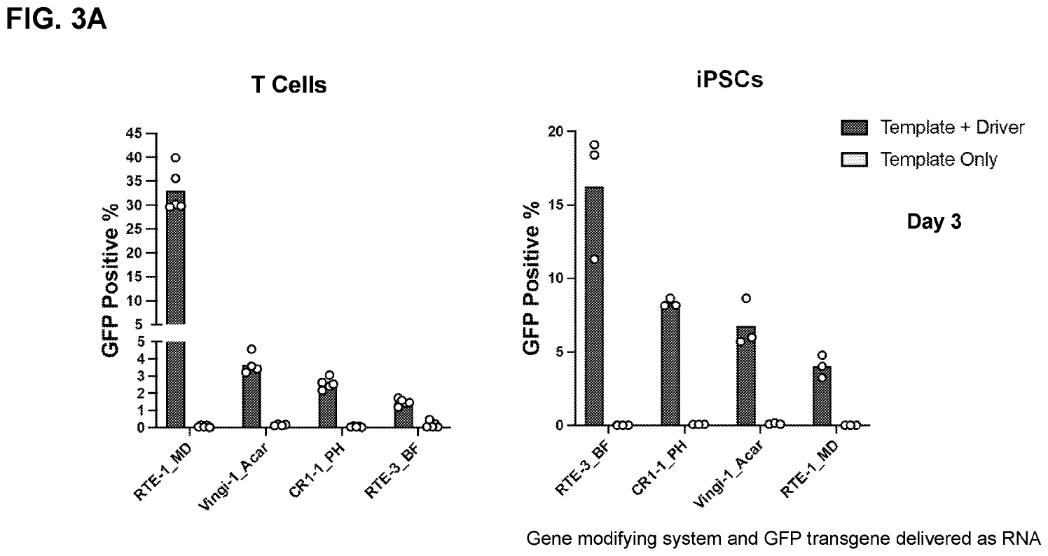

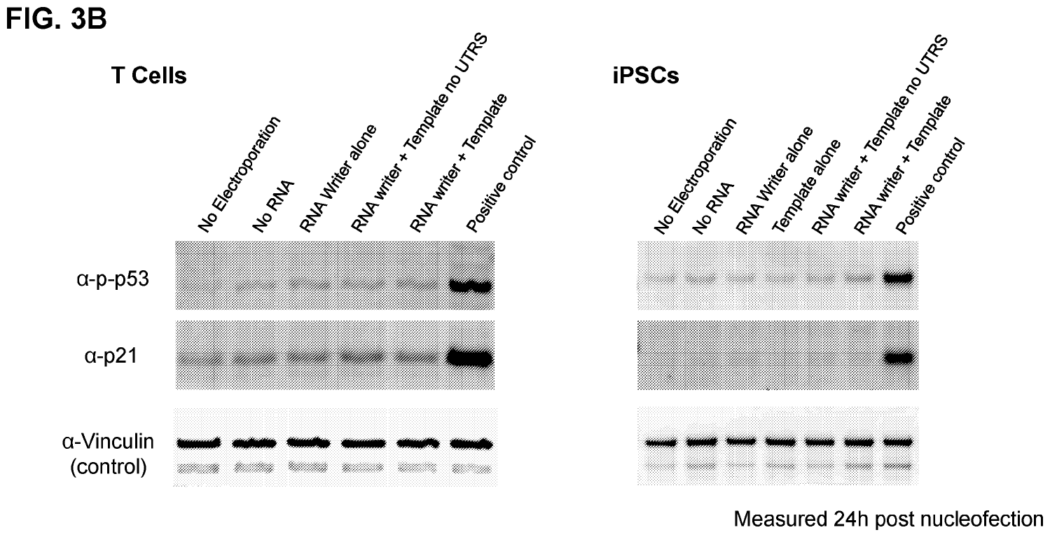

In some embodiments, the template RNA encodes a gene modifying protein in cis with a heterologous object sequence. Various cis constructs were described, for example, in Kuroki-Kami et al (2019) Mobile DNA 10.23 (incorporated by reference herein in its entirety), and can be used in combination with any of the embodiments described herein. For instance, in some embodiments, the template RNA comprises a heterologous object sequence, a sequence encoding a gene modifying protein (e.g., a protein comprising (i) a reverse transcriptase domain and (ii) an endonuclease domain, e.g., as described herein), a 5′ untranslated region, and a 3′ untranslated region. The components may be included in various orders. In some embodiments, the gene modifying protein and heterologous object sequence are encoded in different directions (sense vs. anti-sense), e.g., using an arrangement shown in FIG. 3A of Kuroki-Kami et al, Id. In some embodiments, the gene modifying protein and heterologous object sequence are encoded in the same direction. In some embodiments, the nucleic acid encoding the polypeptide and the template RNA or the nucleic acid encoding the template RNA are covalently linked, e.g., are part of a fusion nucleic acid, and/or are part of the same transcript. In some embodiments, the fusion nucleic acid comprises RNA or DNA.

The nucleic acid encoding the gene modifying polypeptide may, in some instances, be 5′ of the heterologous object sequence. For example, in some embodiments, the template RNA comprises, from 5′ to 3′, a 5′ untranslated region, a sense-encoded gene modifying polypeptide, a sense-encoded heterologous object sequence, and 3′ untranslated region. In some embodiments, the template RNA comprises, from 5′ to 3′, a 5′ untranslated region, a sense-encoded gene modifying polypeptide, anti-sense-encoded heterologous object sequence, and 3′ untranslated region.

It is understood that, when a template RNA is described as comprising an open reading frame or the reverse complement thereof, in some embodiments the template RNA must be converted into double stranded DNA (e.g., through reverse transcription) before the open reading frame can be transcribed and translated.

In certain embodiments, customized RNA sequence template can be identified, designed, engineered and constructed to contain sequences altering or specifying host genome function, for example by introducing a heterologous coding region into a genome; affecting or causing exon structure/alternative splicing; causing disruption of an endogenous gene; causing transcriptional activation of an endogenous gene; causing epigenetic regulation of an endogenous DNA; causing up- or down-regulation of operably liked genes, etc. In certain embodiments, a customized RNA sequence template can be engineered to contain sequences coding for exons and/or transgenes, provide for binding sites to transcription factor activators, repressors, enhancers, etc., and combinations of thereof. In other embodiments, the coding sequence can be further customized with splice acceptor sites, poly-A tails. In certain embodiments the RNA sequence can contain sequences coding for an RNA sequence template homologous to the retrotransposase, be engineered to contain heterologous coding sequences, or combinations thereof.

The template RNA may have some homology to the target DNA. In some embodiments the template RNA has at least 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 175, 200 or more bases of exact homology to the target DNA at the 3′ end of the RNA. In some embodiments the template RNA has at least 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 175, 180, or 200 or more bases of at least 50%, 60%, 70%, 80%, 85%, 90%, 95%, 97%, 98%, 99% or 100% homology to the target DNA, e.g., at the 5′ end of the template RNA. In some embodiments, the template RNA has a 3′ untranslated region derived from a retrotransposon, e.g. a retrotransposons described herein. In some embodiments the template RNA has a 3′ region of at least 10, 15, 20, 25, 30, 40, 50, 60, 80, 100, 120, 140, 160, 180, 200 or more bases of at least 50%, 60%, 70%, 80%, 85%, 90%, 95%, 97%, 98%, 99% or 100% homology to the 3′ sequence of a retrotransposon, e.g., a retrotransposon described herein, e.g. a retrotransposon in Table R1. In some embodiments, the template RNA has a 5′ untranslated region derived from a retrotransposon, e.g. a retrotransposons described herein. In some embodiments the template RNA has a 5′ region of at least 10, 15, 20, 25, 30, 40, 50, 60, 80, 100, 120, 140, 160, 180, or 200 or more bases of at least 40%, 50%, 60%, 70%, 80%, 90%, 95% or greater homology to the 5′ sequence of a retrotransposon, e.g., a retrotransposon described herein, e.g. a retrotransposon described in Table R1.

The template RNA component of a gene modifying system described herein typically is able to bind the gene modifying protein of the system. In some embodiments, the template RNA has a 3′ region that is capable of binding a gene modifying genome editing protein. The binding region, e.g., 3′ region, may be a structured RNA region, e.g., having at least 1, 2 or 3 hairpin loops, capable of binding the gene modifying protein of the system.

The template RNA component of a gene modifying system described herein typically is able to bind the gene modifying protein of the system. In some embodiments, the template RNA has a 5′ region that is capable of binding a gene modifying protein. The binding region, e.g., 5′ region, may be a structured RNA region, e.g., having at least 1, 2 or 3 hairpin loops, capable of binding the gene modifying protein of the system. In some embodiments, the 5′ untranslated region comprises a pseudoknot, e.g., a pseudoknot that is capable of binding to the gene modifying protein.

In some embodiments, the template RNA (e.g., an untranslated region of the hairpin RNA, e.g., a 5′ untranslated region) comprises a stem-loop sequence. In some embodiments, the template RNA (e.g., an untranslated region of the hairpin RNA, e.g., a 5′ untranslated region) comprises a hairpin. In some embodiments, the template RNA (e.g., an untranslated region of the hairpin RNA, e.g., a 5′ untranslated region) comprises a helix. In some embodiments, the template RNA (e.g., an untranslated region of the hairpin RNA, e.g., a 5′ untranslated region) comprises a psuedoknot. In some embodiments, the template RNA comprises a ribozyme. In some embodiments the ribozyme is similar to a hepatitis delta virus (HDV) ribozyme, e.g., has a secondary structure like that of the HDV ribozyme and/or has one or more activities of the HDV ribozyme, e.g., a self-cleavage activity. See, e.g., Eickbush et al., Molecular and Cellular Biology, 2010, 3142-3150.

In some embodiments, the template RNA (e.g., an untranslated region of the hairpin RNA, e.g., a 3′ untranslated region) comprises one or more stem-loops or helices. Exemplary structures of R2 3′ UTRs are shown, for example, in Ruschak et al. “Secondary structure models of the 3′ untranslated regions of diverse R2 RNAs” RNA. 2004 June; 10(6): 978-987, e.g., at FIG. 3, therein, and in Eikbush and Eikbush, “R2 and R2/R1 hybrid non-autonomous retrotransposons derived by internal deletions of full-length elements” Mobile DNA (2012) 3:10; e.g., at FIG. 3 therein, which articles are hereby incorporated by reference in their entirety.

In some embodiments, a template RNA described herein comprises a sequence that is capable of binding to a gene modifying protein described herein. For instance, in some embodiments, the template RNA comprises an MS2 RNA sequence capable of binding to an MS2 coat protein sequence in the gene modifying protein. In some embodiments, the template RNA comprises an RNA sequence capable of binding to a B-box sequence. In some embodiments, in addition to or in place of a UTR, the template RNA is linked (e.g., covalently) to a non-RNA UTR, e.g., a protein or small molecule.

In some embodiments, the template RNA has a poly-A tail at the 3′ end. In some embodiments, the template RNA does not have a poly-A tail at the 3′ end.

In some embodiments the template RNA has a 5′ region of at least 10, 15, 20, 25, 30, 40, 50, 60, 80, 100, 120, 140, 160, 180, 200 or more bases of at least 40%, 50%, 60%, 70%, 80%, 90%, 95% or greater homology to the 5′ sequence of a retrotransposon, e.g., a retrotransposon described herein.

The template RNA of the system typically comprises an object sequence for insertion into a target DNA. The object sequence may be coding or non-coding.

In some embodiments, a system or method described herein comprises a single template RNA. In some embodiments, a system or method described herein comprises a plurality of template RNAs.

In some embodiments, the object sequence may contain an open reading frame. In some embodiments, the template RNA has a Kozak sequence. In some embodiments, the template RNA has an internal ribosome entry site. In some embodiments, the template RNA has a self-cleaving peptide such as a T2A or P2A site. In some embodiments, the template RNA has a start codon. In some embodiments, the template RNA has a splice acceptor site. In some embodiments, the template RNA has a splice donor site. Exemplary splice acceptor and splice donor sites are described in WO2016044416, incorporated herein by reference in its entirety. Exemplary splice acceptor site sequences are known to those of skill in the art and include, by way of example only, CTGACCCTTCTCTCTCTCCCCCAGAG (SEQ ID NO: 15428) (from human HBB gene) and TTTCTCTCCCACAAG (SEQ ID NO: 15429) (from human immunoglobulin-gamma gene). In some embodiments the template RNA, has a microRNA binding site downstream of the stop codon. In some embodiments, the template RNA has a polyA tail downstream of the stop codon of an open reading frame. In some embodiments, the template RNA comprises one or more exons. In some embodiments, the template RNA comprises one or more introns. In some embodiments, the template RNA comprises a eukaryotic transcriptional terminator. In some embodiments, the template RNA comprises an enhanced translation element or a translation enhancing element. In some embodiments, the RNA comprises the human T-cell leukemia virus (HTLV-1) R region. In some embodiments, the RNA comprises a posttranscriptional regulatory element that enhances nuclear export, such as that of Hepatitis B Virus (HPRE) or Woodchuck Hepatitis Virus (WPRE). In some embodiments, in the template RNA, the heterologous object sequence encodes a polypeptide and is coded in an antisense direction with respect to the 5′ and 3′ UTR. In some embodiments, in the template RNA, the heterologous object sequence encodes a polypeptide and is coded in a sense direction with respect to the 5′ and 3′ UTR.

In some embodiments, a nucleic acid described herein (e.g., a template RNA or a DNA encoding a template RNA) comprises a microRNA binding site. In some embodiments, the microRNA binding site is used to increase the target-cell specificity of a gene modifying system. For instance, the microRNA binding site can be chosen on the basis that is recognized by a miRNA that is present in a non-target cell type, but that is not present (or is present at a reduced level relative to the non-target cell) in a target cell type. Thus, when the template RNA is present in a non-target cell, it would be bound by the miRNA, and when the template RNA is present in a target cell, it would not be bound by the miRNA (or bound but at reduced levels relative to the non-target cell). While not wishing to be bound by theory, binding of the miRNA to the template RNA may interfere with insertion of the heterologous object sequence into the genome. Accordingly, the heterologous object sequence would be inserted into the genome of target cells more efficiently than into the genome of non-target cells. A system having a microRNA binding site in the template RNA (or DNA encoding it) may also be used in combination with a nucleic acid encoding a gene modifying polypeptide, wherein expression of the gene modifying polypeptide is regulated by a second microRNA binding site, e.g., as described herein, e.g., in the section entitled “Polypeptide component of gene modifying system.”

In some embodiments, the object sequence may contain a non-coding sequence. For example, the template RNA may comprise a promoter or enhancer sequence. In some embodiments, the template RNA comprises a tissue specific promoter or enhancer, each of which may be unidirectional or bidirectional. In some embodiments, the promoter is an RNA polymerase I promoter, RNA polymerase II promoter, or RNA polymerase III promoter. In some embodiments, the promoter comprises a TATA element. In some embodiments, the promoter comprises a B recognition element. In some embodiments, the promoter has one or more binding sites for transcription factors. In some embodiments, the non-coding sequence is transcribed in an antisense-direction with respect to the 5′ and 3′ UTR. In some embodiments, the non-coding sequence is transcribed in a sense direction with respect to the 5′ and 3′ UTR.

In some embodiments, a nucleic acid described herein (e.g., a template RNA or a DNA encoding a template RNA) comprises a promoter sequence, e.g., a tissue specific promoter sequence. In some embodiments, the tissue-specific promoter is used to increase the target-cell specificity of a gene modifying system. For instance, the promoter can be chosen on the basis that it is active in a target cell type but not active in (or active at a lower level in) a non-target cell type. Thus, even if the promoter integrated into the genome of a non-target cell, it would not drive expression (or only drive low-level expression) of an integrated gene. A system having a tissue-specific promoter sequence in the template RNA may also be used in combination with a microRNA binding site, e.g., in the template RNA or a nucleic acid encoding a gene modifying protein, e.g., as described herein. A system having a tissue-specific promoter sequence in the template RNA may also be used in combination with a DNA encoding a gene modifying polypeptide, driven by a tissue-specific promoter, e.g., to achieve higher levels of gene modifying protein in target cells than in non-target cells.

In some embodiments, a heterologous object sequence comprised by a template RNA (or DNA encoding the template RNA) is operably linked to at least one regulatory sequence. In some embodiments, the heterologous object sequence is operably linked to a tissue-specific promoter, such that expression of the heterologous object sequence, e.g., a therapeutic protein, is upregulated in target cells, as above. In some embodiments, the heterologous object sequence is operably linked to a miRNA binding site, such that expression of the heterologous object sequence, e.g., a therapeutic protein, is downregulated in cells with higher levels of the corresponding miRNA, e.g., non-target cells, as above.

In some embodiments, the template RNA comprises a microRNA sequence, a siRNA sequence, a guide RNA sequence, a piwi RNA sequence.

In some embodiments, the template RNA comprises a non-coding heterologous object sequence, e.g., a regulatory sequence. In some embodiments, integration of the heterologous object sequence thus alters the expression of an endogenous gene. In some embodiments, integration of the heterologous object sequence upregulates expression of an endogenous gene. In some embodiments, integration of the heterologous object sequence downregulated expression of an endogenous gene.

In some embodiments, the template RNA comprises a site that coordinates epigenetic modification. In some embodiments, the template RNA comprises an element that inhibits, e.g., prevents, epigenetic silencing. In some embodiments, the template RNA comprises a chromatin insulator. For example, the template RNA comprises a CTCF site or a site targeted for DNA methylation.

In order to promote higher level or more stable gene expression, the template RNA may include features that prevent or inhibit gene silencing. In some embodiments, these features prevent or inhibit DNA methylation. In some embodiments, these features promote DNA demethylation. In some embodiments, these features prevent or inhibit histone deacetylation. In some embodiments, these features prevent or inhibit histone methylation. In some embodiments, these features promote histone acetylation. In some embodiments, these features promote histone demethylation. In some embodiments, multiple features may be incorporated into the template RNA to promote one or more of these modifications. CpG dinucleotides are subject to methylation by host methyl transferases. In some embodiments, the template RNA is depleted of CpG dinucleotides, e.g., does not comprise CpG nucleotides or comprises a reduced number of CpG dinucleotides compared to a corresponding unaltered sequence. In some embodiments, the promoter driving transgene expression from integrated DNA is depleted of CpG dinucleotides.

In some embodiments, the template RNA comprises a gene expression unit composed of at least one regulatory region operably linked to an effector sequence. The effector sequence may be a sequence that is transcribed into RNA (e.g., a coding sequence or a non-coding sequence such as a sequence encoding a micro RNA).

In some embodiments, the object sequence of the template RNA is inserted into a target genome in an endogenous intron. In some embodiments, the object sequence of the template RNA is inserted into a target genome and thereby acts as a new exon. In some embodiments, the insertion of the object sequence into the target genome results in replacement of a natural exon or the skipping of a natural exon.

In some embodiments, the object sequence of the template RNA is inserted into the target genome in a genomic safe harbor site, such as AAVS1, CCR5, or ROSA26. In some embodiments, the object sequence of the template RNA is inserted into the albumin locus. In some embodiments, the object sequence of the template RNA is inserted into the TRAC locus. In some embodiments, the object sequence of the template RNA is added to the genome in an intergenic or intragenic region. In some embodiments, the object sequence of the template RNA is added to the genome 5′ or 3′ within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb of an endogenous active gene. In some embodiments, the object sequence of the template RNA is added to the genome 5′ or 3′ within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb of an endogenous promoter or enhancer. In some embodiments, the object sequence of the template RNA can be, e.g., 50-50,000 base pairs (e.g., between 50-40,000 bp, between 500-30,000 bp between 500-20,000 bp, between 100-15,000 bp, between 500-10,000 bp, between 50-10,000 bp, between 50-5,000 bp. In some embodiments, the heterologous object sequence is less than 1,000, 1,300, 1500, 2,000, 3,000, 4,000, 5,000, or 7,500 nucleotides in length.

In some embodiments, the genomic safe harbor site is a Natural Harbor™ site. In some embodiments, the Natural Harbor™ site is ribosomal DNA (rDNA). In some embodiments, the Natural Harbor™ site is 5S rDNA, 18S rDNA, 5.8S rDNA, or 28S rDNA. In some embodiments, the Natural Harbor™ site is the Mutsu site in 5S rDNA. In some embodiments, the Natural Harbor™ site is the R2 site, the R5 site, the R6 site, the R4 site, the R1 site, the R9 site, or the RT site in 28S rDNA. In some embodiments, the Natural Harbor™ site is the R8 site or the R7 site in 18S rDNA. In some embodiments, the Natural Harbor™ site is DNA encoding transfer RNA (tRNA). In some embodiments, the Natural Harbor™ site is DNA encoding tRNA-Asp or tRNA-Glu. In some embodiments, the Natural Harbor™ site is DNA encoding spliceosomal RNA. In some embodiments, the Natural Harbor™ site is DNA encoding small nuclear RNA (snRNA) such as U2 snRNA.

Thus, in some aspects, the present disclosure provides a method of inserting a heterologous object sequence into a Natural Harbor™ site. In some embodiments, the method comprises using a gene modifying system described herein, e.g., using a polypeptide of any of Table X, Z1, Z2, 3A, or 3B of PCT Pub. No.: WO/2021/178717 or a polypeptide having sequence similarity thereto, e.g., at least 80%, 85%, 90%, or 95% identity thereto. In some embodiments, the method comprises using an enzyme, e.g., a retrotransposase, to insert the heterologous object sequence into the Natural Harbor™ site. In some aspects, the present disclosure provides a host human cell comprising a heterologous object sequence (e.g., a sequence encoding a therapeutic polypeptide) situated at a Natural Harbor™ site in the genome of the cell. In some embodiments, the Natural Harbor™ site is a site described in Table 4 below. In some embodiments, the heterologous object sequence is inserted within 20, 50, 100, 150, 200, 250, 500, or 1000 base pairs of a sequence shown in Table 4. In some embodiments, the heterologous object sequence is inserted within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb of a sequence shown in Table 4. In some embodiments, the heterologous object sequence is inserted into a site having at least 70%, 75%, 80%, 85%, 90%, 95%, 96%, 97%, 98%, or 99% identity to a sequence shown in Table 4. In some embodiments, the heterologous object sequence is inserted within 20, 50, 100, 150, 200, 250, 500, or 1000 base pairs, or within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb, of a site having at least 70%, 75%, 80%, 85%, 90%, 95%, 96%, 97%, 98%, or 99% identity to a sequence shown in Table 4. In some embodiments, the heterologous object sequence is inserted within a gene indicated in Column 5 of Table 4, or within 20, 50, 100, 150, 200, 250, 500, or 1000 base pairs, or within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb, of the gene.

TABLE 4
Natural Harbor ™ sites. Column 1 indicates a retrotransposon that inserts
into the Natural Harbor ™ site. Column 2 indicates the gene at the Natural Harbor ™ site.
Columns 3 and 4 show exemplary human genome sequence 5′ and 3′ of the insertion site (for
example, 250 bp). Columns 5 and 6 list the example gene symbol and corresponding Gene ID.

| | | | | Example | |
|---|---|---|---|---|---|
| Target | Target | | | Gene | Example |
| Site | Gene | 5′ flanking sequence | 3′ flanking sequence | Symbol | Gene ID |
| R2 | 28S rDNA | CCGGTCCCCCCCGCCGGGTCC | GTAGCCAAATGCCTCGTCATC | RNA28SN1 | 106632264 |
| | | GCCCCCGGGGCCGCGGTTCC | TAATTAGTGACGCGCATGAAT | | |
| | | GCGCGGCGCCTCGCCTCGGC | GGATGAACGAGATTCCCACT | | |
| | | CGGCGCCTAGCAGCCGACTT | GTCCCTACCTACTATCCAGCG | | |
| | | AGAACTGGTGCGGACCAGGG | AAACCACAGCCAAGGGAACG | | |
| | | GAATCCGACTGTTTAATTAAA | GGCTTGGCGGAATCAGCGGG | | |
| | | ACAAAGCATCGCGAAGGCCC | GAAAGAAGACCCTGTTGAGC | | |
| | | GCGGCGGGTGTTGACGCGAT | TTGACTCTAGTCTGGCACGGT | | |
| | | GTGATTTCTGCCCAGTGCTCT | GAAGAGACATGAGAGGTGTA | | |
| | | GAATGTCAAAGTGAAGAAAT | GAATAAGTGGGAGGCCCCCG | | |
| | | TCAATGAAGCGCGGGTAAAC | GCGCCCCCCCGGTGTCCCCGC | | |
| | | GGCGGGAGTAACTATGACTC | GAGGGGCCCGGGGGGGGGT | | |
| | | TCTTAAG (SEQ ID NO: | CCGCCG (SEQ ID NO: 1513) | | |
| | | 1508) | | | |
| R4 | 28S rDNA | GCGGTTCCGCGCGGCGCCTC | CGCATGAATGGATGAACGAG | RNA28SN1 | 106632264 |
| | | GCCTCGGCCGGCGCCTAGCA | ATTCCCACTGTCCCTACCTACT | | |
| | | GCCGACTTAGAACTGGTGCG | ATCCAGCGAAACCACAGCCA | | |
| | | GACCAGGGGAATCCGACTGT | AGGGAACGGGCTTGGCGGA | | |
| | | TTAATTAAAACAAAGCATCGC | ATCAGCGGGGAAAGAAGACC | | |
| | | GAAGGCCCGCGGCGGGTGTT | CTGTTGAGCTTGACTCTAGTC | | |
| | | GACGCGATGTGATTTCTGCCC | TGGCACGGTGAAGAGACATG | | |
| | | AGTGCTCTGAATGTCAAAGT | AGAGGTGTAGAATAAGTGGG | | |
| | | GAAGAAATTCAATGAAGCGC | AGGCCCCCGGCGCCCCCCCG | | |
| | | GGGTAAACGGCGGGAGTAAC | GTGTCCCCGCGAGGGGCCCG | | |
| | | TATGACTCTCTTAAGGTAGCC | GGGCGGGGTCCGCCGGCCCT | | |
| | | AAATGCCTCGTCATCTAATTA | GCGGGCCGCCGGTGAAATAC | | |
| | | GTGACG (SEQ ID NO: | CACTACTC (SEQ ID NO: | | |
| | | 1509) | 1514) | | |
| R5 | 28S rDNA | TCCCCCCCGCCGGGTCCGCCC | CCAAATGCCTCGTCATCTAAT | RNA28SN1 | 106632264 |
| | | CCGGGGCCGCGGTTCCGCGC | TAGTGACGCGCATGAATGGA | | |
| | | GGCGCCTCGCCTCGGCCGGC | TGAACGAGATTCCCACTGTCC | | |
| | | GCCTAGCAGCCGACTTAGAA | CTACCTACTATCCAGCGAAAC | | |
| | | CTGGTGCGGACCAGGGGAAT | CACAGCCAAGGGAACGGGCT | | |
| | | CCGACTGTTTAATTAAAACAA | TGGCGGAATCAGCGGGGAAA | | |
| | | AGCATCGCGAAGGCCCGCGG | GAAGACCCTGTTGAGCTTGA | | |
| | | CGGGTGTTGACGCGATGTGA | CTCTAGTCTGGCACGGTGAA | | |
| | | TTTCTGCCCAGTGCTCTGAAT | GAGACATGAGAGGTGTAGAA | | |
| | | GTCAAAGTGAAGAAATTCAA | TAAGTGGGAGGCCCCCGGCG | | |
| | | TGAAGCGCGGGTAAACGGCG | CCCCCCCGGTGTCCCCGCGAG | | |
| | | GGAGTAACTATGACTCTCTTA | GGGCCCGGGGCGGGGTCCG | | |
| | | AGGTAG (SEQ ID NO: | CCGGCCC (SEQ ID NO: 1515) | | |
| | | 1510) | | | |
| R9 | 28S rDNA | CGGCGCGCTCGCCGGCCGAG | TAGCTGGTTCCCTCCGAAGTT | RNA28SN1 | 106632264 |
| | | GTGGGATCCCGAGGCCTCTC | TCCCTCAGGATAGCTGGCGCT | | |
| | | CAGTCCGCCGAGGGCGCACC | CTCGCAGACCCGACGCACCCC | | |
| | | ACCGGCCCGTCTCGCCCGCCG | CGCCACGCAGTTTTATCCGGT | | |
| | | CGCCGGGGAGGTGGAGCAC | AAAGCGAATGATTAGAGGTC | | |
| | | GAGCGCACGTGTTAGGACCC | TTGGGGCCGAAACGATCTCA | | |
| | | GAAAGATGGTGAACTATGCC | ACCTATTCTCAAACTTTAAAT | | |
| | | TGGGCAGGGCGAAGCCAGA | GGGTAAGAAGCCCGGCTCGC | | |
| | | GGAAACTCTGGTGGAGGTCC | TGGCGTGGAGCCGGGCGTGG | | |
| | | GTAGCGGTCCTGACGTGCAA | AATGCGAGTGCCTAGTGGGC | | |
| | | ATCGGTCGTCCGACCTGGGT | CACTTTTGGTAAGCAGAACTG | | |
| | | ATAGGGGCGAAAGACTAATC | GCGCTGCGGGATGAACCGAA | | |
| | | GAACCATCTAG (SEQ ID NO: | CGCC (SEQ ID NO: 1516) | | |
| | | 1511) | | | |
| R8 | 18S rDNA | GCATTCGTATTGCGCCGCTAG | TGAAACTTAAAGGAATTGAC | RNA18SN1 | 106631781 |
| | | AGGTGAAATTCTTGGACCGG | GGAAGGGCACCACCAGGAGT | | |
| | | CGCAAGACGGACCAGAGCGA | GGAGCCTGCGGCTTAATTTG | | |
| | | AAGCATTTGCCAAGAATGTTT | ACTCAACACGGGAAACCTCA | | |
| | | TCATTAATCAAGAACGAAAGT | CCCGGCCCGGACACGGACAG | | |
| | | CGGAGGTTCGAAGACGATCA | GATTGACAGATTGATAGCTCT | | |
| | | GATACCGTCGTAGTTCCGACC | TTCTCGATTCCGTGGGTGGTG | | |
| | | ATAAACGATGCCGACCGGCG | GTGCATGGCCGTTCTTAGTTG | | |
| | | ATGCGGCGGCGTTATTCCCAT | GTGGAGCGATTTGTCTGGTT | | |
| | | GACCCGCCGGGCAGCTTCCG | AATTCCGATAACGAACGAGA | | |
| | | GGAAACCAAAGTCTTTGGGT | CTCTGGCATGCTAACTAGTTA | | |
| | | TCCGGGGGGAGTATGGTTGC | CGCGACCCCCGAGCGGTCGG | | |
| | | AAAGC (SEQ ID NO: 1512) | CGTCCC (SEQ ID NO: 1517) | | |
| R4- | tRNA-Asp | | | TRD-GTC1-1 | 100189207 |
| 2_SRa | | | | | |
| LIN25_ | tRNA-Glu | | | TRE-CTC1-1 | 100189384 |
| SM | | | | | |
| R1 | 28S rDNA | TAGCAGCCGACTTAGAACTG | ACCTACTATCCAGCGAAACCA | RNA28SN1 | 106632264 |
| | | GTGCGGACCAGGGGAATCCG | CAGCCAAGGGAACGGGCTTG | | |
| | | ACTGTTTAATTAAAACAAAGC | GCGGAATCAGCGGGGAAAG | | |
| | | ATCGCGAAGGCCCGCGGCGG | AAGACCCTGTTGAGCTTGACT | | |
| | | GTGTTGACGCGATGTGATTTC | CTAGTCTGGCACGGTGAAGA | | |
| | | TGCCCAGTGCTCTGAATGTCA | GACATGAGAGGTGTAGAATA | | |
| | | AAGTGAAGAAATTCAATGAA | AGTGGGAGGCCCCCGGCGCC | | |
| | | GCGCGGGTAAACGGCGGGA | CCCCCGGTGTCCCCGCGAGG | | |
| | | GTAACTATGACTCTCTTAAGG | GGCCCGGGGCGGGGTCCGCC | | |
| | | TAGCCAAATGCCTCGTCATCT | GGCCCTGCGGGCCGCCGGTG | | |
| | | AATTAGTGACGCGCATGAAT | AAATACCACTACTCTGATCGT | | |
| | | GGATGAACGAGATTCCCACT | TTTTTCACTGACCCGGTGAGG | | |
| | | GTCCCT (SEQ ID NO: | CGGGGGG (SEQ ID NO: | | |
| | | 1518) | 1524) | | |
| R6 | 28S rDNA | CCCCCCGCCGGGTCCGCCCCC | AAATGCCTCGTCATCTAATTA | RNA28SN1 | 106632264 |
| | | GGGGCCGCGGTTCCGCGCGG | GTGACGCGCATGAATGGATG | | |
| | | CGCCTCGCCTCGGCCGGCGC | AACGAGATTCCCACTGTCCCT | | |
| | | CTAGCAGCCGACTTAGAACT | ACCTACTATCCAGCGAAACCA | | |
| | | GGTGCGGACCAGGGGAATCC | CAGCCAAGGGAACGGGCTTG | | |
| | | GACTGTTTAATTAAAACAAAG | GCGGAATCAGCGGGGAAAG | | |
| | | CATCGCGAAGGCCCGCGGCG | AAGACCCTGTTGAGCTTGACT | | |
| | | GGTGTTGACGCGATGTGATT | CTAGTCTGGCACGGTGAAGA | | |
| | | TCTGCCCAGTGCTCTGAATGT | GACATGAGAGGTGTAGAATA | | |
| | | CAAAGTGAAGAAATTCAATG | AGTGGGAGGCCCCCGGCGCC | | |
| | | AAGCGCGGGTAAACGGCGG | CCCCCGGTGTCCCCGCGAGG | | |
| | | GAGTAACTATGACTCTCTTAA | GGCCCGGGGCGGGGTCCGCC | | |
| | | GGTAGCC (SEQ ID NO: | GGCCCTG (SEQ ID NO: 1525) | | |
| | | 1519) | | | |
| R7 | 18S rDNA | GCGCAAGACGGACCAGAGCG | GGAGCCTGCGGCTTAATTTG | RNA18SN1 | 106631781 |
| | | AAAGCATTTGCCAAGAATGTT | ACTCAACACGGGAAACCTCA | | |
| | | TTCATTAATCAAGAACGAAAG | CCCGGCCCGGACACGGACAG | | |
| | | TCGGAGGTTCGAAGACGATC | GATTGACAGATTGATAGCTCT | | |
| | | AGATACCGTCGTAGTTCCGAC | TTCTCGATTCCGTGGGTGGTG | | |
| | | CATAAACGATGCCGACCGGC | GTGCATGGCCGTTCTTAGTTG | | |
| | | GATGCGGCGGCGTTATTCCC | GTGGAGCGATTTGTCTGGTT | | |
| | | ATGACCCGCCGGGCAGCTTC | AATTCCGATAACGAACGAGA | | |
| | | CGGGAAACCAAAGTCTTTGG | CTCTGGCATGCTAACTAGTTA | | |
| | | GTTCCGGGGGGAGTATGGTT | CGCGACCCCCGAGCGGTCGG | | |
| | | GCAAAGCTGAAACTTAAAGG | CGTCCCCCAACTTCTTAGAGG | | |
| | | AATTGACGGAAGGGCACCAC | GACAAGTGGCGTTCAGCCAC | | |
| | | CAGGAGT (SEQ ID NO: | CCGAG (SEQ ID NO: 1526) | | |
| | | 1520) | | | |
| RT | 28S rDNA | GGCCGGGCGCGACCCGCTCC | AACTGGCTTGTGGCGGCCAA | RNA28SN1 | 106632264 |
| | | GGGGACAGTGCCAGGTGGG | GCGTTCATAGCGACGTCGCTT | | |
| | | GAGTTTGACTGGGGCGGTAC | TTTGATCCTTCGATGTCGGCT | | |
| | | ACCTGTCAAACGGTAACGCA | CTTCCTATCATTGTGAAGCAG | | |
| | | GGTGTCCTAAGGCGAGCTCA | AATTCACCAAGCGTTGGATTG | | |
| | | GGGAGGACAGAAACCTCCCG | TTCACCCACTAATAGGGAACG | | |
| | | TGGAGCAGAAGGGCAAAAG | TGAGCTGGGTTTAGACCGTC | | |
| | | CTCGCTTGATCTTGATTTTCA | GTGAGACAGGTTAGTTTTACC | | |
| | | GTACGAATACAGACCGTGAA | CTACTGATGATGTGTTGTTGC | | |
| | | AGCGGGGCCTCACGATCCTTC | CATGGTAATCCTGCTCAGTAC | | |
| | | TGACCTTTTGGGTTTTAAGCA | GAGAGGAACCGCAGGTTCAG | | |
| | | GGAGGTGTCAGAAAAGTTAC | ACATTTGGTGTATGTGCTTGG | | |
| | | CACAGGGAT (SEQ ID NO: | C (SEQ ID NO: 1527) | | |
| | | 1521) | | | |
| Mutsu | 5S rDNA | GTCTACGGCCATACCACCC | TGAACGCGCCCGATCTCGTCT | RNA5S1 | 100169751 |
| | | (SEQ ID NO: 1522) | GATCTCGGAAGCTAAGCAGG | | |
| | | | GTCGGGCCTGGTTAGTACTT | | |
| | | | GGATGGGAGACCGCCTGGGA | | |
| | | | ATACCGGGTGCTGTAGGCTTT | | |
| | | | (SEQ ID NO: 1528) | | |
| Utopia/ | U2 snRNA | ATCGCTTCTCGGCCTTTTGGC | TCTGTTCTTATCAGTTTAATAT | RNU2-1 | 6066 |
| Keno | | TAAGATCAAGTGTAGTA (SEQ | CTGATACGTCCTCTATCCGAG | | |
| | | ID NO: 1523) | GACAATATATTAAATGGATTT | | |
| | | | TTGGAGCAGGGAGATGGAAT | | |
| | | | AGGAGCTTGCTCCGTCCACTC | | |
| | | | CACGCATCGACCTGGTATTGC | | |
| | | | AGTACCTCCAGGAACGGTGC | | |
| | | | ACCC (SEQ ID NO: 1529) | | |

In some embodiments, a system or method described herein results in insertion of a heterologous sequence into a target site in the human genome. In some embodiments, the target site in the human genome has sequence similarity to the corresponding target site of the corresponding wild-type retrotransposase (e.g., the retrotransposase from which the gene modifying polypeptide was derived) in the genome of the organism to which it is native. For instance, in some embodiments, the identity between the 40 nucleotides of human genome sequence centered at the insertion site and the 40 nucleotides of native organism genome sequence centered at the insertion site is less than 99.5%, 99%, 98%, 97%, 96%, 95%, 90%, 85%, 80%, 75%, 70%, 60%, or 50%, or is between 50-60%, 60-70%, 70-80%, 80-90%, or 90-100%. In some embodiments, the identity between the 100 nucleotides of human genome sequence centered at the insertion site and the 100 nucleotides of native organism genome sequence centered at the insertion site is less than 99.5%, 99%, 98%, 97%, 96%, 95%, 90%, 85%, 80%, 75%, 70%, 60%, or 50%, or is between 50-60%, 60-70%, 70-80%, 80-90%, or 90-100%. In some embodiments, the identity between the 500 nucleotides of human genome sequence centered at the insertion site and the 500 nucleotides of native organism genome sequence centered at the insertion site is less than 99.5%, 99%, 98%, 97%, 96%, 95%, 90%, 85%, 80%, 75%, 70%, 60%, or 50%, or is between 50-60%, 60-70%, 70-80%, 80-90%, or 90-100%.

The template nucleic acid (e.g., template RNA) component of a gene modifying system described herein typically is able to bind the gene modifying protein of the system. In some embodiments, the template nucleic acid (e.g., template RNA) has a 3′ region that is capable of binding a gene modifying protein. The binding region, e.g., 3′ region, may be a structured RNA region, e.g., having at least 1, 2 or 3 hairpin loops, capable of binding the gene modifying protein of the system. The binding region may associate the template nucleic acid (e.g., template RNA) with any of the polypeptide modules. In some embodiments, the binding region of the template nucleic acid (e.g., template RNA) may associate with an RNA-binding domain in the polypeptide. In some embodiments, the binding region of the template nucleic acid (e.g., template RNA) may associate with the reverse transcription domain of the polypeptide (e.g., specifically bind to the RT domain). For example, where the reverse transcription domain is derived from a non-LTR retrotransposon, the template nucleic acid (e.g., template RNA) may contain a binding region derived from a non-LTR retrotransposon, e.g., a 3′ UTR from a non-LTR retrotransposon. In some embodiments a system or method described herein comprises a single template nucleic acid (e.g., template RNA). In some embodiments a system or method described herein comprises a plurality of template nucleic acids (e.g., template RNAs). In some embodiments, when the system comprises a plurality of nucleic acids, each nucleic acid comprises a conjugating domain. In some embodiments, a conjugating domain enables association of nucleic acid molecules, e.g., by hybridization of complementary sequences.

In some embodiments, the template nucleic acid may comprise one or more UTRs (e.g., a 5′ UTR or a 3′ UTR, e.g., from an R2-type retrotransposon). In some embodiments, the UTR facilitates interaction of the template with the reverse transcriptase domain of the polypeptide. In some embodiments, the template possesses one or more sequences aiding in association of the template with the gene modifying polypeptide. In some embodiments, these sequences may be derived from retrotransposon UTRs. In some embodiments, the UTRs may be located flanking the desired insertion sequence. In some embodiments, a sequence with target site homology may be located outside of one or both UTRs. In some embodiments, the sequence with target site homology can anneal to the target sequence to prime reverse transcription. In some embodiments, the 5′ and/or 3′ UTR may be located terminal to the target site homology sequence. In some embodiments, the gene modifying system may result in the insertion of a desired payload without any additional sequence (e.g., a gene expression unit without UTRs used to bind the gene modifying protein).

In some embodiments, the template RNA comprises one or more of the following sequences from 5′ to 3′: 5′ UTR, polyadenylation signal, an object sequence for incorporation into the genome (e.g., a sequence encoding a CAR), Kozak sequence, promoter, 3′ UTR. In some embodiments, the template RNA comprises one or more of the following sequences from 5′ to 3′: 5′ UTR, bGHpA, an object sequence for incorporation into the genome (e.g., a sequence encoding a CAR), Kozak sequence, EF1a short promoter, 3′ UTR. In some embodiments, the template RNA comprises one or more of the following sequences from 5′ to 3′: 5′ UTR, bGHpA, WPRE, an object sequence for incorporation into the genome (e.g., a sequence encoding a CAR), Kozak sequence, EF1a short promoter, 3′ UTR. In some embodiments, the template RNA comprises one or more of the following sequences from 5′ to 3′: 5′ UTR, bGHpA, an object sequence for incorporation into the genome (e.g., a sequence encoding a CAR), Kozak sequence, MIND promoter, 3′ UTR. In some embodiments, the template RNA comprises one or more of the following sequences from 5′ to 3′: 5′ UTR, bGHpA, WPRE, an object sequence for incorporation into the genome (e.g., a sequence encoding a CAR), Kozak sequence, MND promoter, 3′ UTR.

In some embodiments, the template RNA comprises a 5′ UTR. In some embodiments, the template RNA comprises bGHpA. In some embodiments, the template RNA comprises WPRE. In some embodiments, the template RNA comprises a Kozak sequence. In some embodiments, the template RNA comprises an EF1a short promoter. In some embodiments, the template RNA comprises an MIND promoter. In some embodiments, the template RNA comprises a 3′ UTR.

In some embodiments, the template RNA comprises one or more of the following sequences from 5′ to 3′: 5′ UTR, TKpA, an object sequence for incorporation into the genome (e.g., a sequence encoding a CAR), Kozak sequence, EF1a short promoter or MIND promoter, 3′ UTR.

In some embodiments, the template RNA comprises a 5′ UTR. In some embodiments, the template RNA comprises TKpA. In some embodiments, the template RNA comprises a Kozak sequence. In some embodiments, the template RNA comprises an EF1a short promoter. In some embodiments, the template RNA comprises an MND promoter. In some embodiments, the template RNA comprises a 3′ UTR.

In some embodiments, the template RNA comprises a safety gene or switch, such as, for example, a caspase (e.g., caspase-9 or iCasp-9) or RQR8. In some embodiments, the template RNA comprises one or more of the following sequences from 5′ to 3′: 5′ UTR, bGHpA, WPRE, an object sequence for incorporation into the genome (e.g., a sequence encoding a CAR), RQR8, Kozak sequence, MND promoter, 3′ UTR. In some embodiments, the template RNA comprises one or more of the following sequences from 5′ to 3′: 5′ UTR, bGHpA, WPRE, an object sequence for incorporation into the genome (e.g., a sequence encoding a CAR), RQR8, Kozak sequence, MND promoter, 3′ UTR. In some embodiments, the RQR8 comprises an amino acid sequence according to SEQ ID NO: 15453, or a sequence having at least 80%, 90%, 95%, or 99% identity thereto.

The template nucleic acid (e.g., template RNA) can be designed to result in insertions, mutations, or deletions at the target DNA locus. In some embodiments, the template nucleic acid (e.g., template RNA) may be designed to cause an insertion in the target DNA. For example, the template nucleic acid (e.g., template RNA) may contain a heterologous sequence, wherein the reverse transcription will result in insertion of the heterologous sequence into the target DNA. In other embodiments, the RNA template may be designed to write a deletion into the target DNA. For example, the template nucleic acid (e.g., template RNA) may match the target DNA upstream and downstream of the desired deletion, wherein the reverse transcription will result in the copying of the upstream and downstream sequences from the template nucleic acid (e.g., template RNA) without the intervening sequence, e.g., causing deletion of the intervening sequence. In other embodiments, the template nucleic acid (e.g., template RNA) may be designed to write an edit into the target DNA. For example, the template RNA may match the target DNA sequence with the exception of one or more nucleotides, wherein the reverse transcription will result in the copying of these edits into the target DNA, e.g., resulting in mutations, e.g., transition or transversion mutations.

In some embodiments, a gene modifying system is capable of producing an insertion into the target site of at least 45, 50, 55, 60, 65, 70, 75, 80, 85, 90, 95, or 100 nucleotides (and optionally no more than 500, 400, 300, 200, or 100 nucleotides). In some embodiments, a gene modifying system is capable of producing an insertion into the target site of at least 1, 2, 3, 4, 5, 6, 7, 8, 9, 10, 15, 20, 25, 30, 35, 40, 45, 50, 55, 60, 65, 70, 75, 80, 85, 90, 95, or 100 nucleotides (and optionally no more than 500, 400, 300, 200, or 100 nucleotides). In some embodiments, a gene modifying system is capable of producing an insertion into the target site of at least 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5 or 10 kilobases (and optionally no more than 1, 5, 10, or 20 kilobases). In some embodiments, a gene modifying system is capable of producing a deletion of at least 81, 85, 90, 95, 100, 110, 120, 130, 140, 150, 160, 170, 180, 190, or 200 nucleotides (and optionally no more than 500, 400, 300, or 200 nucleotides). In some embodiments, a gene modifying system is capable of producing a deletion of at least 81, 85, 90, 95, 100, 110, 120, 130, 140, 150, 160, 170, 180, 190, or 200 nucleotides (and optionally no more than 500, 400, 300, or 200 nucleotides). In some embodiments, a gene modifying system is capable of producing a deletion of at least 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200 nucleotides (and optionally no more than 500, 400, 300, or 200 nucleotides). In some embodiments, a gene modifying system is capable of producing a deletion of at least 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5 or 10 kilobases (and optionally no more than 1, 5, 10, or 20 kilobases).

### Methods and Compositions for Modified RNA (e.g., Template RNA)

In some embodiments, an RNA component of the system (e.g., a template RNA, as described herein) comprises one or more nucleotide modifications. In some embodiments, the modification pattern of the template RNA can significantly affect in vivo activity compared to unmodified or end-modified guides. Without wishing to be bound by theory, this process may be due, at least in part, to a stabilization of the RNA conferred by the modifications. Non-limiting examples of such modifications may include 2′-O-methyl (2′-O-Me), 2′-O-(2-methoxyethyl) (2′-O-MOE), 2′-fluoro (2′-F), phosphorothioate (PS) bond between nucleotides, G-C substitutions, and inverted abasic linkages between nucleotides and equivalents thereof.

In some embodiments, the template RNA (e.g., at the portion thereof that binds a target site) comprises a 5′ terminus region. In some embodiments, the template RNA does not comprise a 5′ terminus region. In some embodiments, the 5′ terminus region comprises a 5′ end modification. In some embodiments, the template RNA comprises a 2′-O-methyl (2′-O-Me) modified nucleotide. In some embodiments, the template RNA comprises a 2′-O-(2-methoxy ethyl) (2′-O-moe) modified nucleotide. In some embodiments, the template RNA comprises a 2′-fluoro (2′-F) modified nucleotide. In some embodiments, the template RNA comprises a phosphorothioate (PS) bond between nucleotides. In some embodiments, the template RNA comprises a 5′ end modification, a 3′ end modification, or 5′ and 3′ end modifications. In some embodiments, the 5′ end modification comprises a phosphorothioate (PS) bond between nucleotides. In some embodiments, the 5′ end modification comprises a 2′-O-methyl (2′-O-Me), 2′-O-(2-methoxy ethyl) (2′-O-MOE), and/or 2′-fluoro (2′-F) modified nucleotide. In some embodiments, the 5′ end modification comprises at least one phosphorothioate (PS) bond and one or more of a 2′-O-methyl (2′-O-Me), 2′-O-(2-methoxyethyl) (2′-O-MOE), and/or 2′-fluoro (2′-F) modified nucleotide. The end modification may comprise a phosphorothioate (PS), 2′-O-methyl (2′-O-Me), 2′-O-(2-methoxyethyl) (2′-O-MOE), and/or 2′-fluoro (2′-F) modification. Equivalent end modifications are also encompassed by embodiments described herein. In some embodiments, the template RNA comprises an end modification in combination with a modification of one or more regions of the template RNA. In some embodiments, structure-guided and systematic approaches are used to introduce modifications (e.g., 2′-OMe-RNA, 2′-F-RNA, and PS modifications) to a template RNA, for example, as described in Mir et al. Nat Commun 9:2641 (2018) (incorporated by reference herein in its entirety). In some embodiments, the incorporation of 2′-F-RNAs increases thermal and nuclease stability of RNA:RNA or RNA:DNA duplexes, e.g., while minimally interfering with C3′-endo sugar puckering. In some embodiments, 2′-F may be better tolerated than 2′-OMe at positions where the 2′-OH is important for RNA:DNA duplex stability. In some embodiments, structure-guided and systematic approaches (e.g., as described in Mir et al. Nat Commun 9:2641 (2018); incorporated herein by reference in its entirety) are employed to find modifications for the template RNA. In some embodiments, a structure of polypeptide bound to template RNA is used to determine non-protein-contacted nucleotides of the RNA that may then be selected for modifications, e.g., with lower risk of disrupting the association of the RNA with the polypeptide. Secondary structures in a template RNA can also be predicted in silico by software tools, e.g., the RNAstructure tool available at rna.urmc.rochester.edu/RNAstructureWeb (Bellaousov et al. Nucleic Acids Res 41:W471-W474 (2013); incorporated by reference herein in its entirety), e.g., to determine secondary structures for selecting modifications, e.g., hairpins, stems, and/or bulges.

It is contemplated that it may be useful to employ circular and/or linear RNA states during the formulation, delivery, or gene modifying reaction within the target cell. Thus, in some embodiments of any of the aspects described herein, a gene modifying system comprises one or more circular RNAs (circRNAs). In some embodiments of any of the aspects described herein, a gene modifying system comprises one or more linear RNAs. In some embodiments, a nucleic acid as described herein (e.g., a template nucleic acid, a nucleic acid molecule encoding a gene modifying polypeptide, or both) is a circRNA. In some embodiments, a circular RNA molecule encodes the gene modifying polypeptide. In some embodiments, the circRNA molecule encoding the gene modifying polypeptide is delivered to a host cell. In some embodiments, the circRNA molecule encoding the gene modifying polypeptide is linearized (e.g., in the host cell, e.g., in the nucleus of the host cell) prior to translation.

Circular RNAs (circRNAs) have been found to occur naturally in cells and have been found to have diverse functions, including both non-coding and protein coding roles in human cells. It has been shown that a circRNA can be engineered by incorporating a self-splicing intron into an RNA molecule (or DNA encoding the RNA molecule) that results in circularization of the RNA, and that an engineered circRNA can have enhanced protein production and stability (Wesselhoeft et al. Nature Communications 2018). In some embodiments, the gene modifying polypeptide is encoded as circRNA. In certain embodiments, the template nucleic acid is a DNA, such as a dsDNA or ssDNA.

In some embodiments, the circRNA comprises one or more ribozyme sequences. In some embodiments, the ribozyme sequence is activated for autocleavage, e.g., in a host cell, e.g., thereby resulting in linearization of the circRNA. In some embodiments, the ribozyme is activated when the concentration of magnesium reaches a sufficient level for cleavage, e.g., in a host cell. In some embodiments, the circRNA is maintained in a low magnesium environment prior to delivery to the host cell. In some embodiments, the ribozyme is a protein-responsive ribozyme. In some embodiments, the ribozyme is a nucleic acid-responsive ribozyme. In some embodiments, the circRNA comprises a cleavage site. In some embodiments, the circRNA comprises a second cleavage site.

In some embodiments, the circRNA is linearized in the nucleus of a target cell. In some embodiments, linearization of a circRNA in the nucleus of a cell involves components present in the nucleus of the cell, e.g., to activate a cleavage event. For example, the B2 and ALU retrotransposons contain self-cleaving ribozymes whose activity is enhanced by interaction with the Polycomb protein, EZH2 (Hernandez et al. PNAS 117(1):415-425 (2020)). Thus, in some embodiments, a ribozyme, e.g., a ribozyme from a B2 or ALU element, that is responsive to a nuclear element, e.g., a nuclear protein, e.g., a genome-interacting protein, e.g., an epigenetic modifier, e.g., EZH2, is incorporated into a circRNA, e.g., of a gene modifying system. In some embodiments, nuclear localization of the circRNA results in an increase in autocatalytic activity of the ribozyme and linearization of the circRNA.

In some embodiments, the ribozyme is heterologous to one or more of the other components of the gene modifying system. In some embodiments, an inducible ribozyme (e.g., in a circRNA as described herein) is created synthetically, for example, by utilizing a protein ligand-responsive aptamer design. A system for utilizing the satellite RNA of tobacco ringspot virus hammerhead ribozyme with an MS2 coat protein aptamer has been described (Kennedy et al. Nucleic Acids Res 42(19):12306-12321 (2014), incorporated herein by reference in its entirety) that results in activation of the ribozyme activity in the presence of the MS2 coat protein. In embodiments, such a system responds to protein ligand localized to the cytoplasm or the nucleus. In some embodiments, the protein ligand is not MS2. Methods for generating RNA aptamers to target ligands have been described, for example, based on the systematic evolution of ligands by exponential enrichment (SELEX) (Tuerk and Gold, Science 249(4968):505-510 (1990); Ellington and Szostak, Nature 346(6287):818-822 (1990); the methods of each of which are incorporated herein by reference) and have, in some instances, been aided by in silico design (Bell et al. PNAS 117(15):8486-8493, the methods of which are incorporated herein by reference). Thus, in some embodiments, an aptamer for a target ligand is generated and incorporated into a synthetic ribozyme system, e.g., to trigger ribozyme-mediated cleavage and circRNA linearization, e.g., in the presence of the protein ligand. In some embodiments, circRNA linearization is triggered in the cytoplasm, e.g., using an aptamer that associates with a ligand in the cytoplasm. In some embodiments, circRNA linearization is triggered in the nucleus, e.g., using an aptamer that associates with a ligand in the nucleus. In embodiments, the ligand in the nucleus comprises an epigenetic modifier or a transcription factor. In some embodiments, the ligand that triggers linearization is present at higher levels in on-target cells than off-target cells.

It is further contemplated that a nucleic acid-responsive ribozyme system can be employed for circRNA linearization. For example, biosensors that sense defined target nucleic acid molecules to trigger ribozyme activation are described, e.g., in Penchovsky (Biotechnology Advances 32(5):1015-1027 (2014), incorporated herein by reference). By these methods, a ribozyme naturally folds into an inactive state and is only activated in the presence of a defined target nucleic acid molecule (e.g., an RNA molecule). In some embodiments, a circRNA of a gene modifying system comprises a nucleic acid-responsive ribozyme that is activated in the presence of a defined target nucleic acid, e.g., an RNA, e.g., an mRNA. In some embodiments, the nucleic acid that triggers linearization is present at higher levels in on-target cells than off-target cells.

In some embodiments of any of the aspects herein, a gene modifying system incorporates one or more ribozymes with inducible specificity to a target tissue or target cell of interest, e.g., a ribozyme that is activated by a ligand or nucleic acid present at higher levels in a target tissue or target cell of interest. In some embodiments, the gene modifying system incorporates a ribozyme with inducible specificity to a subcellular compartment, e.g., the nucleus, nucleolus, cytoplasm, or mitochondria. In some embodiments, the ribozyme that is activated by a ligand or nucleic acid present at higher levels in the target subcellular compartment. In some embodiments, an RNA component of a gene modifying system is provided as circRNA, e.g., that is activated by linearization. In some embodiments, linearization of a circRNA encoding a gene modifying polypeptide activates the molecule for translation. In some embodiments, a signal that activates a circRNA component of a gene modifying system is present at higher levels in on-target cells or tissues, e.g., such that the system is specifically activated in these cells.

In some embodiments, an RNA component of a gene modifying system is provided as a circRNA that is inactivated by linearization. In some embodiments, a circRNA encoding the gene modifying polypeptide is inactivated by cleavage and degradation. In some embodiments, a circRNA encoding the gene modifying polypeptide is inactivated by cleavage that separates a translation signal from the coding sequence of the polypeptide. In some embodiments, a signal that inactivates a circRNA component of a gene modifying system is present at higher levels in off-target cells or tissues, such that the system is specifically inactivated in these cells.

Further included here are compositions and methods for the assembly of full or partial template RNA molecules. In some embodiments, RNA molecules may be assembled by the connection of two or more (e.g., two, three, four, five, six, seven, eight, nine, ten, or more) RNA segments with each other. In an aspect, the disclosure provides methods for producing nucleic acid molecules, the methods comprising contacting two or more linear RNA segments with each other under conditions that allow for the 5′ terminus of a first RNA segment to be covalently linked with the 3′ terminus of a second RNA segment. In some embodiments, the joined molecule may be contacted with a third RNA segment under conditions that allow for the 5′ terminus of the joined molecule to be covalently linked with the 3′ terminus of the third RNA segment. In embodiments, the method further comprises joining a fourth, fifth, or additional RNA segments to the elongated molecule. This form of assembly may, in some instances, allow for rapid and efficient assembly of RNA molecules.

The disclosure also provides compositions and methods for the production of template RNA molecules with specificity for a gene modifying polypeptide. In an aspect, the method comprises: (1) identification of the target site and desired modification thereto, (2) production of RNA segments including a heterologous object sequence segment and a gene modifying polypeptide binding motif, and/or (3) connection of the four or more segments into at least one molecule, e.g., into a single RNA molecule. In some embodiments, some or all of the template RNA segments comprised in (2) are assembled into a template RNA molecule, e.g., one, two, three, or four of the listed components. In some embodiments, the segments comprised in (2) may be produced in further segmented molecules, e.g., split into at least 2, at least 3, at least 4, or at least 5 or more sub-segments, e.g., that are subsequently assembled, e.g., by one or more methods described herein.

In some embodiments, RNA segments may be produced by chemical synthesis. In some embodiments, RNA segments may be produced by in vitro transcription of a nucleic acid template, e.g., by providing an RNA polymerase to act on a cognate promoter of a DNA template to produce an RNA transcript. In some embodiments, in vitro transcription is performed using, e.g., a T7, T3, or SP6 RNA polymerase, or a derivative thereof, acting on a DNA, e.g., dsDNA, ssDNA, linear DNA, plasmid DNA, linear DNA amplicon, linearized plasmid DNA, e.g., encoding the RNA segment, e.g., under transcriptional control of a cognate promoter, e.g., a T7, T3, or SP6 promoter. In some embodiments, a combination of chemical synthesis and in vitro transcription is used to generate the RNA segments for assembly. In embodiments, the gene modifying polypeptide binding segments are produced by chemical synthesis and the heterologous object sequence segment is produced by in vitro transcription. Without wishing to be bound by theory, in vitro transcription may be better suited for the production of longer RNA molecules. In some embodiments, reaction temperature for in vitro transcription may be lowered, e.g., be less than 37° C. (e.g., between 0-10 C, 10-20 C, or 20-30 C), to result in a higher proportion of full-length transcripts (Krieg Nucleic Acid Res 18:6463 (1990)). In some embodiments, a protocol for improved synthesis of long transcripts is employed to synthesize a long template RNA, e.g., a template RNA greater than 5 kb, such as the use of e.g., T7 RiboMAX Express, which can generate 27 kb transcripts in vitro (Thiel et al. J Gen Virol 82(6):1273-1281 (2001)). In some embodiments, modifications to RNA molecules as described herein may be incorporated during synthesis of RNA segments (e.g., through the inclusion of modified nucleotides or alternative binding chemistries), following synthesis of RNA segments through chemical or enzymatic processes, following assembly of one or more RNA segments, or a combination thereof.

In some embodiments, an mRNA of the system (e.g., an mRNA encoding a gene modifying polypeptide) is synthesized in vitro using T7 polymerase-mediated DNA-dependent RNA transcription from a linearized DNA template, where UTP is optionally substituted with 1-methylpseudoUTP. In some embodiments, the transcript incorporates 5′ and 3′ UTRs, e.g., GGGAAAUAAGAGAGAAAAGAAGAGUAAGAAGAAAUAUAAGAGCCACC (SEQ ID NO: 15430) and UGAUAAUAGGCUGGAGCCUCGGUGGCCAUGCUUCUUGCCCCUUGGGCCUCCCCCC AGCCCCUCCUCCCCUUCCUGCACCCGUACCCCCGUGGUCUUUGAAUAAAGUCUGA (SEQ ID NO: 15431), or functional fragments or variants thereof, and optionally includes a poly-A tail, which can be encoded in the DNA template or added enzymatically following transcription. In some embodiments, a donor methyl group, e.g., S-adenosylmethionine, is added to a methylated capped RNA with cap 0 structure to yield a cap 1 structure that increases mRNA translation efficiency (Richner et al. Cell 168(6): P1114-1125 (2017)).

In some embodiments, the transcript from a T7 promoter starts with a GGG motif. In some embodiments, a transcript from a T7 promoter does not start with a GGG motif. It has been shown that a GGG motif at the transcriptional start, despite providing superior yield, may lead to T7 RNAP synthesizing a ladder of poly(G) products as a result of slippage of the transcript on the three C residues in the template strand from +1 to +3 (Imburgio et al. Biochemistry 39(34):10419-10430 (2000). For tuning transcription levels and altering the transcription start site nucleotides to fit alternative 5′ UTRs, the teachings of Davidson et al. Pac Symp Biocomput 433-443 (2010) describe T7 promoter variants, and the methods of discovery thereof, that fulfill both of these traits.

In some embodiments, RNA segments may be connected to each other by covalent coupling. In some embodiments, an RNA ligase, e.g., T4 RNA ligase, may be used to connect two or more RNA segments to each other. When a reagent such as an RNA ligase is used, a 5′ terminus is typically linked to a 3′ terminus. In some embodiments, if two segments are connected, then there are two possible linear constructs that can be formed (i.e., (1) 5′-Segment 1-Segment 2-3′ and (2) 5′-Segment 2-Segment 1-3′). In some embodiments, intramolecular circularization can also occur. Both of these issues can be addressed, for example, by blocking one 5′ terminus or one 3′ terminus so that RNA ligase cannot ligate the terminus to another terminus. In embodiments, if a construct of 5′-Segment 1-Segment 2-3′ is desired, then placing a blocking group on either the 5′ end of Segment 1 or the 3′ end of Segment 2 may result in the formation of only the correct linear ligation product and/or prevent intramolecular circularization. Compositions and methods for the covalent connection of two nucleic acid (e.g., RNA) segments are disclosed, for example, in US20160102322A1 (incorporated herein by reference in its entirety), along with methods including the use of an RNA ligase to directionally ligate two single-stranded RNA segments to each other.

One example of an end blocker that may be used in conjunction with, for example, T4 RNA ligase, is a dideoxy terminator. T4 RNA ligase typically catalyzes the ATP-dependent ligation of phosphodiester bonds between 5′-phosphate and 3′-hydroxyl termini. In some embodiments, when T4 RNA ligase is used, suitable termini must be present on the termini being ligated. One means for blocking T4 RNA ligase on a terminus comprises failing to have the correct terminus format. Generally, termini of RNA segments with a 5-hydroxyl or a 3′-phosphate will not act as substrates for T4 RNA ligase.

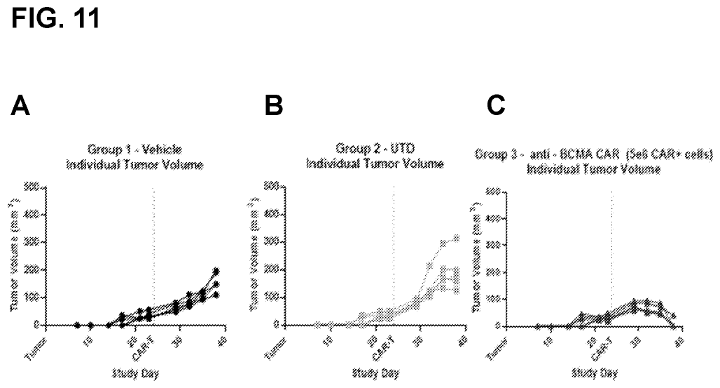

Additional exemplary methods that may be used to connect RNA segments is by click chemistry (e.g., as described in U.S. Pat. Nos. 7,375,234 and 7,070,941, and US Patent Publication No. 2013/0046084, the entire disclosures of which are incorporated herein by reference). For example, one exemplary click chemistry reaction is between an alkyne group and an azide group (see FIG. 11 of US20160102322A1, which is incorporated herein by reference in its entirety). Any click reaction may potentially be used to link RNA segments (e.g., Cu-azide-alkyne, strain-promoted-azide-alkyne, staudinger ligation, tetrazine ligation, photo-induced tetrazole-alkene, thiol-ene, NHS esters, epoxides, isocyanates, and aldehyde-aminooxy). In some embodiments, ligation of RNA molecules using a click chemistry reaction is advantageous because click chemistry reactions are fast, modular, efficient, often do not produce toxic waste products, can be done with water as a solvent, and/or can be set up to be stereospecific.

In some embodiments, RNA segments may be connected using an Azide-Alkyne Huisgen Cycloaddition. reaction, which is typically a 1,3-dipolar cycloaddition between an azide and a terminal or internal alkyne to give a 1,2,3-triazole for the ligation of RNA segments. Without wishing to be bound by theory, one advantage of this ligation method may be that this reaction can initiated by the addition of required Cu(I) ions. Other exemplary mechanisms by which RNA segments may be connected include, without limitation, the use of halogens (F—, Br—, I—)/alkynes addition reactions, carbonyls/sulfhydryls/maleimide, and carboxyl/amine linkages. For example, one RNA molecule may be modified with thiol at 3′ (using disulfide amidite and universal support or disulfide modified support), and the other RNA molecule may be modified with acrydite at 5′ (using acrylic phosphoramidite), then the two RNA molecules can be connected by a Michael addition reaction. This strategy can also be applied to connecting multiple RNA molecules stepwise. Also provided are methods for linking more than two (e.g., three, four, five, six, etc.) RNA molecules to each other. Without wishing to be bound by theory, this may be useful when a desired RNA molecule is longer than about 40 nucleotides, e.g., such that chemical synthesis efficiency degrades, e.g., as noted in US20160102322A1 (incorporated herein by reference in its entirety).

RNA molecules may be produced, for example, by processes such as in vitro transcription or chemical synthesis. In some embodiments, when chemical synthesis is used to produce such RNA molecules, they may be produced as a single synthesis product or by linking two or more synthesized RNA segments to each other. In embodiments, when three or more RNA segments are connected to each other, different methods may be used to link the individual segments together. Also, the RNA segments may be connected to each other in one pot (e.g., a container, vessel, well, tube, plate, or other receptacle), all at the same time, or in one pot at different times or in different pots at different times. In a non-limiting example, to assemble RNA Segments 1, 2 and 3 in numerical order, RNA Segments 1 and 2 may first be connected, 5′ to 3′, to each other. The reaction product may then be purified for reaction mixture components (e.g., by chromatography), then placed in a second pot, for connection of the 3′ terminus with the 5′ terminus of RNA Segment 3. The final reaction product may then be connected to the 5′ terminus of RNA Segment 3.

A number of additional linking chemistries may be used to connect RNA segments according to method of the invention. Some of these chemistries are set out in Table 6 of US20160102322A1, which is incorporated herein by reference in its entirety.

### Additional Template Features

In some embodiments, the template (e.g., template RNA) comprises certain structural features, e.g., determined in silico. In embodiments, the template RNA is predicted to have minimal energy structures between −280 and −480 kcal/mol (e.g., between −280 to −300, −300 to −350, −350 to −400, −400 to −450, or −450 to −480 kcal/mol), e.g., as measured by RNAstructure, e.g., as described in Turner and Mathews Nucleic Acids Res 38:D280-282 (2009) (incorporated herein by reference in its entirety).

In some embodiments, the template (e.g., template RNA) comprises certain structural features, e.g., determined in vitro. In embodiments, the template RNA is sequence optimized, e.g., to reduce secondary structure as determined in vitro, for example, by SHAPE-MaP (e.g., as described in Siegfried et al. Nat Methods 11:959-965 (2014); incorporated herein by reference in its entirety). In some embodiments, the template (e.g., template RNA) comprises certain structural features, e.g., determined in cells. In embodiments, the template RNA is sequence optimized, e.g., to reduce secondary structure as measured in cells, for example, by DMS-MaPseq (e.g., as described in Zubradt et al. Nat Methods 14:75-82 (2017); incorporated by reference herein in its entirety).

### Additional Functional Characteristics and Features of Gene Modifying Systems

A gene modifying system as described herein may, in some instances, be characterized by one or more functional measurements or characteristics. In some embodiments, the DNA binding domain has one or more of the functional characteristics described below. In some embodiments, the RNA binding domain has one or more of the functional characteristics described below. In some embodiments, the endonuclease domain has one or more of the functional characteristics described below. In some embodiments, the reverse transcriptase domain has one or more of the functional characteristics described below. In some embodiments, the template (e.g., template RNA) has one or more of the functional characteristics described below. In some embodiments, the target site bound by the gene modifying polypeptide has one or more of the functional characteristics described below.

### Gene Modifying Polypeptide

### DNA Binding Domain

In some embodiments, the DNA binding domain is capable of binding to a target sequence (e.g., a dsDNA target sequence) with greater affinity than a reference DNA binding domain. In some embodiments, the reference DNA binding domain is a DNA binding domain from R2_BM of B. mori. In some embodiments, the DNA binding domain is capable of binding to a target sequence (e.g., a dsDNA target sequence) with an affinity between 100 pM-10 nM (e.g., between 100 pM-1 nM or 1 nM-10 nM).

In some embodiments, the affinity of a DNA binding domain for its target sequence (e.g., dsDNA target sequence) is measured in vitro, e.g., by thermophoresis, e.g., as described in Asmari et al. Methods 146:107-119 (2018) (incorporated by reference herein in its entirety).

In embodiments, the DNA binding domain is capable of binding to its target sequence (e.g., dsDNA target sequence), e.g, with an affinity between 100 pM-10 nM (e.g., between 100 pM-1 nM or 1 nM-10 nM) in the presence of a molar excess of scrambled sequence competitor dsDNA, e.g., of about 100-fold molar excess.

In some embodiments, the DNA binding domain is found associated with its target sequence (e.g., dsDNA target sequence) more frequently than any other sequence in the genome of a target cell, e.g., human target cell, e.g., as measured by ChIP-seq (e.g., in HEK293T cells), e.g., as described in He and Pu (2010) Curr. Protoc Mol Biol Chapter 21 (incorporated herein by reference in its entirety). In some embodiments, the DNA binding domain is found associated with its target sequence (e.g., dsDNA target sequence) at least about 5-fold or 10-fold, more frequently than any other sequence in the genome of a target cell, e.g., as measured by ChIP-seq (e.g., in HEK293T cells), e.g., as described in He and Pu (2010), supra.

In some embodiments, a gene modifying polypeptide comprises a modification to a DNA-binding domain, e.g., relative to the wild-type polypeptide. In some embodiments, the DNA-binding domain comprises an addition, deletion, replacement, or modification to the amino acid sequence of the original DNA-binding domain. In some embodiments, the DNA-binding domain is modified to include a heterologous functional domain that binds specifically to a target nucleic acid (e.g., DNA) sequence of interest. In some embodiments, the functional domain replaces at least a portion (e.g., the entirety of) the prior DNA-binding domain of the polypeptide. In some embodiments, a gene modifying polypeptide comprises a modification to an endonuclease domain, e.g., relative to the wild-type polypeptide. In some embodiments, the endonuclease domain comprises an addition, deletion, replacement, or modification to the amino acid sequence of the original endonuclease domain. In some embodiments, the endonuclease domain is modified to include a heterologous functional domain that binds specifically to and/or induces endonuclease cleavage of a target nucleic acid (e.g., DNA) sequence of interest.

### RNA Binding Domain

In some embodiments, the RNA binding domain is capable of binding to a template RNA with greater affinity than a reference RNA binding domain. In some embodiments, the reference RNA binding domain is an RNA binding domain from R2_BM of B. mori. In some embodiments, the RNA binding domain is capable of binding to a template RNA with an affinity between 100 pM-10 nM (e.g., between 100 pM-1 nM or 1 nM-10 nM). In some embodiments, the affinity of a RNA binding domain for its template RNA is measured in vitro, e.g., by thermophoresis, e.g., as described in Asmari et al. Methods 146:107-119 (2018) (incorporated by reference herein in its entirety). In some embodiments, the affinity of a RNA binding domain for its template RNA is measured in cells (e.g., by FRET or CLIP-Seq).

In some embodiments, the RNA binding domain is associated with the template RNA in vitro at a frequency at least about 5-fold or 10-fold higher than with a scrambled RNA. In some embodiments, the frequency of association between the RNA binding domain and the template RNA or scrambled RNA is measured by CLIP-seq, e.g., as described in Lin and Miles (2019) Nucleic Acids Res 47(11):5490-5501 (incorporated by reference herein in its entirety). In some embodiments, the RNA binding domain is associated with the template RNA in cells (e.g., in HEK293T cells) at a frequency at least about 5-fold or 10-fold higher than with a scrambled RNA. In some embodiments, the frequency of association between the RNA binding domain and the template RNA or scrambled RNA is measured by CLIP-seq, e.g., as described in Lin and Miles (2019), supra.

### Endonuclease Domain

In some embodiments, the endonuclease domain is associated with the target dsDNA in vitro at a frequency at least about 5-fold or 10-fold higher than with a scrambled dsDNA. In some embodiments, the endonuclease domain is associated with the target dsDNA in vitro at a frequency at least about 5-fold or 10-fold higher than with a scrambled dsDNA, e.g., in a cell (e.g., a HEK293T cell). In some embodiments, the frequency of association between the endonuclease domain and the target DNA or scrambled DNA is measured by ChIP-seq, e.g., as described in He and Pu (2010) Curr. Protoc Mol Biol Chapter 21 (incorporated by reference herein in its entirety).

In some embodiments, the endonuclease domain can catalyze the formation of a nick at a target sequence, e.g., to an increase of at least about 5-fold or 10-fold relative to a non-target sequence (e.g., relative to any other genomic sequence in the genome of the target cell). In some embodiments, the level of nick formation is determined using NickSeq, e.g., as described in Elacqua et al. (2019) bioRxiv doi.org/10.1101/867937 (incorporated herein by reference in its entirety).

In some embodiments, the endonuclease domain is capable of nicking DNA in vitro. In embodiments, the nick results in an exposed base. In embodiments, the exposed base can be detected using a nuclease sensitivity assay, e.g., as described in Chaudhry and Weinfeld (1995) Nucleic Acids Res 23(19):3805-3809 (incorporated by reference herein in its entirety). In embodiments, the level of exposed bases (e.g., detected by the nuclease sensitivity assay) is increased by at least 10%, 50%, or more relative to a reference endonuclease domain. In some embodiments, the reference endonuclease domain is an endonuclease domain from R2_BM of B. mori.

In some embodiments, the endonuclease domain is capable of nicking DNA in a cell. In embodiments, the endonuclease domain is capable of nicking DNA in a HEK293T cell. In embodiments, an unrepaired nick that undergoes replication in the absence of Rad51 results in increased NHEJ rates at the site of the nick, which can be detected, e.g., by using a Rad51 inhibition assay, e.g., as described in Bothmer et al. (2017) Nat Commun 8:13905 (incorporated by reference herein in its entirety). In embodiments, NHEJ rates are increased above 0-5%. In embodiments, NHEJ rates are increased to 20-70% (e.g., between 30%-60% or 40-50%), e.g., upon Rad51 inhibition.

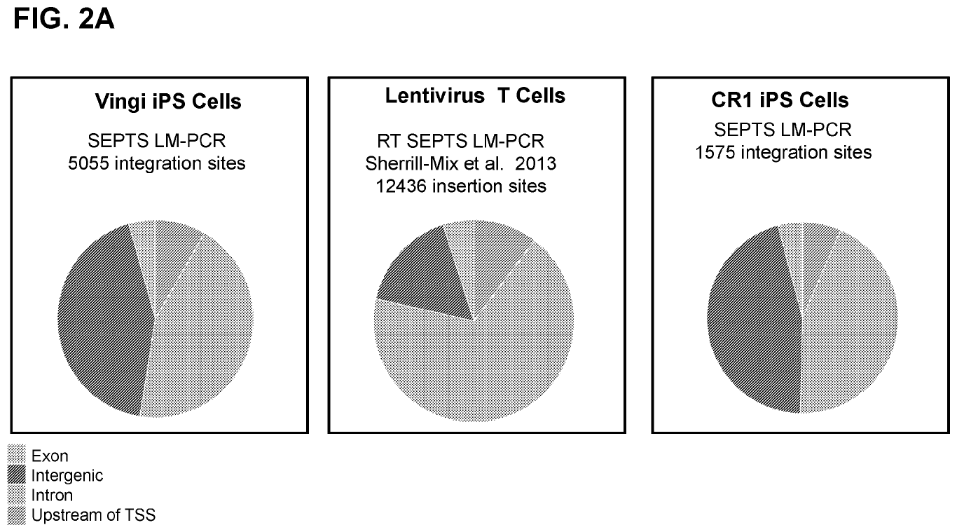

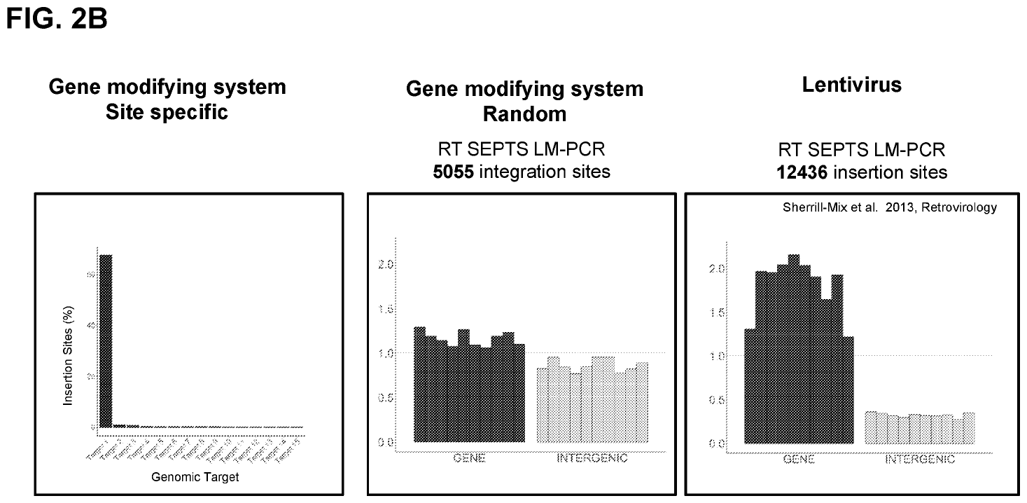

In some embodiments, the endonuclease domain releases the target after cleavage. In some embodiments, release of the target is indicated indirectly by assessing for multiple turnovers by the enzyme, e.g., as described in Yourik at al. RNA 25(1):35-44 (2019) (incorporated herein by reference in its entirety) and shown in FIG. 2. In some embodiments, the kₑₓₚ of an endonuclease domain is 1×10⁻³-1×10⁻⁵ min−1 as measured by such methods.

In some embodiments, the endonuclease domain has a catalytic efficiency (k_{cat}/Kₘ) greater than about 1×10⁸ s⁻¹ M⁻¹ in vitro. In embodiments, the endonuclease domain has a catalytic efficiency greater than about 1×10⁵, 1×10⁶, 1×10⁷, or 1×10⁸, s⁻¹ M⁻¹ in vitro. In embodiments, catalytic efficiency is determined as described in Chen et al. (2018) Science 360(6387):436-439 (incorporated herein by reference in its entirety). In some embodiments, the endonuclease domain has a catalytic efficiency (k_{cat}/Kₘ) greater than about 1×10⁸ s⁻¹ M⁻¹ in cells. In embodiments, the endonuclease domain has a catalytic efficiency greater than about 1×10⁵, 1×10⁶, 1×10⁷, or 1×10⁸ s⁻¹ M⁻¹ in cells.

### Reverse Transcriptase Domain

In some embodiments, the reverse transcriptase domain has a lower probability of premature termination rate (P_{off}) in vitro relative to a reference reverse transcriptase domain. In some embodiments, the reference reverse transcriptase domain is a reverse transcriptase domain from R2_BM of B. mori or a viral reverse transcriptase domain, e.g., the RT domain from M-MLV.

In some embodiments, the reverse transcriptase domain has a lower probability of premature termination rate (P_{off}) in vitro of less than about 5×10⁻³/nt, 5×10⁻⁴/nt, or 5×10⁻⁶/nt, e.g., as measured on a 1094 nt RNA. In embodiments, the in vitro premature termination rate is determined as described in Bibillo and Eickbush (2002) J Biol Chem 277(38):34836-34845 (incorporated by reference herein its entirety).

In some embodiments, the reverse transcriptase domain is able to complete at least about 30% or 50% of integrations in cells. The percent of complete integrations can be measured by dividing the number of substantially full-length integration events (e.g., genomic sites that comprise at least 98% of the expected integrated sequence) by the number of total (including substantially full-length and partial) integration events in a population of cells. In embodiments, the integrations in cells is determined (e.g., across the integration site) using long-read amplicon sequencing, e.g., as described in Karst et al. (2020) bioRxiv doi.org/10.1101/645903 (incorporated by reference herein its in entirety).

In embodiments, quantifying integrations in cells comprises counting the fraction of integrations that contain at least about 75%, 80%, 85%, 90%, 95%, 96%, 97%, 98%, 99%, or 100% of the DNA sequence corresponding to the template RNA (e.g., a template RNA having a length of at least 0.05, 0.1, 0.5, 0.6, 0.7, 0.8, 0.9, 1, 1.5, 2, 3, 4, or 5 kb, e.g., a length between 0.5-0.6, 0.6-0.7, 0.7-0.8, 0.8-0.9, 1.0-1.2, 1.2-1.4, 1.4-1.6, 1.6-1.8, 1.8-2.0, 2-3, 3-4, or 4-5 kb).

In some embodiments, the reverse transcriptase domain is capable of polymerizing dNTPs in vitro. In embodiments, the reverse transcriptase domain is capable of polymerizing dNTPs in vitro at a rate between 0.1-50 nt/sec (e.g., between 0.1-1, 1-10, or 10-50 nt/sec). In embodiments, polymerization of dNTPs by the reverse transcriptase domain is measured by a single-molecule assay, e.g., as described in Schwartz and Quake (2009) PNAS 106(48):20294-20299 (incorporated by reference in its entirety).

In some embodiments, the reverse transcriptase domain has an in vitro error rate (e.g., misincorporation of nucleotides) of between 1×10⁻³-1×10⁻⁴ or 1×10⁻⁴-1×10⁻⁵ substitutions/nt, e.g., as described in Yasukawa et al. (2017) Biochem Biophys Res Commun 492(2):147-153 (incorporated herein by reference in its entirety). In some embodiments, the reverse transcriptase domain has an error rate (e.g., misincorporation of nucleotides) in cells (e.g., HEK293T cells) of between 1×10⁻³-1×10⁻⁴ or 1×10⁻⁴-1×10⁻⁵ substitutions/nt, e.g., by long-read amplicon sequencing, e.g., as described in Karst et al. (2020) bioRxiv doi.org/10.1101/645903 (incorporated by reference herein in its entirety).

In some embodiments, the reverse transcriptase domain is capable of performing reverse transcription of a target RNA in vitro. In some embodiments, the reverse transcriptase requires a primer of at least 3 nt to initiate reverse transcription of a template. In some embodiments, reverse transcription of the target RNA is determined by detection of cDNA from the target RNA (e.g., when provided with a ssDNA primer, e.g., which anneals to the target with at least 3, 4, 5, 6, 7, 8, 9, or 10 nt at the 3′ end), e.g., as described in Bibillo and Eickbush (2002) J Biol Chem 277(38):34836-34845 (incorporated herein by reference in its entirety).

In some embodiments, the reverse transcriptase domain performs reverse transcription at least 5 or 10 times more efficiently (e.g., by cDNA production), e.g., when converting its RNA template to cDNA, for example, as compared to an RNA template lacking the protein binding motif (e.g., a 3′ UTR). In embodiments, efficiency of reverse transcription is measured as described in Yasukawa et al. (2017) Biochem Biophys Res Commun 492(2):147-153 (incorporated by reference herein in its entirety).

In some embodiments, the reverse transcriptase domain specifically binds a specific RNA template with higher frequency (e.g., about 5 or 10-fold higher frequency) than any endogenous cellular RNA, e.g., when expressed in cells (e.g., HEK293T cells). In embodiments, frequency of specific binding between the reverse transcriptase domain and the template RNA are measured by CLIP-seq, e.g., as described in Lin and Miles (2019) Nucleic Acids Res 47(11):5490-5501 (incorporated herein by reference in its entirety).

### Target Site and Integration

In some embodiments, after gene editing, the target site surrounding the integrated sequence contains a limited number of insertions or deletions, for example, in less than about 50% or 10% of integration events, e.g., as determined by long-read amplicon sequencing of the target site, e.g., as described in Karst et al. (2020) bioRxiv doi.org/10.1101/645903 (incorporated by reference herein in its entirety). In some embodiments, the target site does not show multiple insertion events, e.g., head-to-tail or head-to-head duplications, e.g., as determined by long-read amplicon sequencing of the target site, e.g., as described in Karst et al. bioRxiv doi.org/10.1101/645903 (2020) (incorporated herein by reference in its entirety). In some embodiments, the target site contains an integrated sequence corresponding to the template RNA. In some embodiments, the target site does not contain insertions resulting from endogenous RNA in more than about 1% or 10% of events, e.g., as determined by long-read amplicon sequencing of the target site, e.g., as described in Karst et al. bioRxiv doi.org/10.1101/645903 (2020) (incorporated herein by reference in its entirety). In some embodiments, the target site contains the integrated sequence corresponding to the template RNA.

In some embodiments, the target site contains an integrated sequence corresponding to the template RNA. In embodiments, the target site does not comprise sequence outside of the template, e.g., as determined by long-read amplicon sequencing of the target site (for example, as described in Karst et al. bioRxiv doi.org/10.1101/645903 (2020); incorporated herein by reference in its entirety).

In some embodiments, the heterologous object sequence is integrated upstream of a gene (e.g., within 2 kb or 10 kb of a transcription start site (TSS)), within a coding portion of a gene (e.g., an exon), within a non-coding portion of a gene (e.g., an intron), or an intergenic location (e.g., downstream of a gene). In some embodiments, within a plurality of cells containing a plurality of copies of a gene encoded by the heterologous object sequence, less than or equal to 70%, 65%, 60%, or 55% of copies of the heterologous object sequence (e.g., gene encoding a CAR) are situated within a gene endogenous to a cell of the population. In some embodiments, less than 10%, 9%, 8%, 7%, 6%, or 5% of copies of the heterologous object sequence (e.g., gene encoding a CAR) are situated within an exon of a gene endogenous to a cell of the population. In some embodiments, less than 10%, 9%, 8%, 7%, 6%, or 5% of copies of the heterologous object sequence (e.g., gene encoding a CAR) are situated within a coding region of a gene endogenous to a cell of the population. In some embodiments, less than 70%, 65%, 60%, 55%, or 50% of copies of the heterologous object sequence (e.g., gene encoding a CAR) are situated within an intron of a gene endogenous to a cell of the population. In some embodiments, less than 70%, 65%, 60%, 55%, or 50% of copies of the heterologous object sequence (e.g., gene encoding a CAR) are situated within a non-coding region of a gene endogenous to a cell of the population. In some embodiments, less than 10%, 9%, 8%, or 7% of copies of the heterologous object sequence (e.g., gene encoding a CAR) are situated upstream of a gene (e.g., within 2 kb of a transcriptional start site (TSS)) endogenous to a cell of the population. In some embodiments, at least 20%, 25%, 30%, 35%, or 40% of copies of the heterologous object sequence (e.g., gene encoding a CAR) are situated within an intergenic region endogenous to a cell of the population. In some embodiments, at least 20%, 25%, 30%, 35%, or 40% of copies of the heterologous object sequence (e.g., gene encoding a CAR) are situated downstream of a gene endogenous to a cell of the population.

In some embodiments, modifying a genome of a cell using a gene modifying system described herein results in a higher level of insertions in an intergenic location compared to a lentiviral system. Without wishing to be bound by theory, the integration pattern of the gene modifying systems is advantageous because, in some embodiments, it is desired to reduce disrupting expression of endogenous genes in the host cell.

### DNA Damage Response

In some embodiments, modifying a genome of a cell (e.g., a primary cell, e.g., a T cell or an IPSC) using a gene modifying system does not result in activation of the endogenous DNA damage response (DDR) pathway. In some embodiments, modifying a genome of a cell (e.g., a primary cell, e.g., an IPSC) using a gene modifying system results in activation of the cell's endogenous DDR pathway less than in an otherwise similar cell treated with Cas9, e.g., in an assay according to Example 2.

In some embodiments, modifying a genome of a cell (e.g., a primary cell, e.g., a T cell or an IPSC) using a gene modifying system does not result in activation of the endogenous interferon response. In some embodiments, modifying a genome of a cell (e.g., a primary cell, e.g., a T cell or an IPSC) using a gene modifying system results in activation of the cell's interferon response less than in an otherwise similar cell treated with a gene modifying system comprising elements from a LINE-1 retrotransposase, e.g., in an assay according to Example 3.

### Self-Inactivating Modules for Regulating Gene Modifying Activity

In some embodiments, the gene modifying polypeptide systems described herein includes a self-inactivating module. The self-inactivating module leads to a decrease of expression of the gene modifying polypeptide, the gene modifying template, or both. Without wishing to be bound by the theory, the self-inactivating module provides for a temporary period of gene modifying polypeptide expression prior to inactivation. Without wishing to be bound by theory, the activity of the gene modifying polypeptide at a target site introduces a mutation (e.g. a substitution, insertion, or deletion) into the DNA encoding the gene modifying polypeptide or gene modifying template which results in a decrease of gene modifying polypeptide or template expression. In some embodiments of the self-inactivating module, a target site for the gene modifying polypeptide is included in the DNA encoding the gene modifying polypeptide or gene modifying template. In some embodiments, one, two, three, four, five, or more copies of the target site are included in the DNA encoding the gene modifying polypeptide or gene modifying template. In some embodiments, the target site in the DNA encoding the gene modifying polypeptide or gene modifying template is the same target site as the target site on the genome. In some embodiments, the target site is a different target site than the target site on the genome. In some embodiments, the self-inactivation module target site uses the same or a different template RNA as the genome target site. In some embodiments, the target side is nicked. The target site may be incorporated into an enhancer, a promoter, an untranslated region, an exon, an intron, an open reading frame, or a stuffer sequence.

In some embodiments, upon inactivation, the decrease of expression is 25%, 50%, 60%, 70%, 80%, 90%, 95%, 99%, 99.9%, or more lower than a gene modifying system that does not contain the self-inactivating module. In some embodiments, a gene modifying system that contains the self-inactivating module has a 5%, 10%, 15%, 20%, 25%, 30%, 40%, 50%, 60%, 70%, 80%, 90%, 95% 99%, or higher rate of integrations in target sites than off-target sites compared to a gene modifying system that does not contain the self-inactivation module. A gene modifying system that contains the self-inactivating module has a 10%, 15%, 20%, 25%, 30%, 40%, 50%, 60%, 70%, 80%, 90%, 95% 99%, or higher efficiency of target site modification compared to a gene modifying system that does not contain the self-inactivation module. In some embodiments, the self-inactivating module is included when the gene modifying polypeptide is delivered as DNA, e.g. via a viral vector.

Self-inactivating modules have been described for nucleases. See, e.g. in Li et al A Self-Deleting AAV-CRISPR System for In Vivo Genome Editing, Mol Ther Methods Clin Dev. 2019 Mar. 15; 12: 111-122, P. Singhal, Self-Inactivating Cas9: a method for reducing exposure while maintaining efficacy in virally delivered Cas9 applications (available at editasmedicine.com/wp-content/uploads/2019/10/aef_asgct_poster_2017_final_-_present_5-11-17_515pm1_1494537387_1494558495_1497467403.pdf), and Epstein and Schaffer Engineering a Self-Inactivating CRISPR System for AAV Vectors Targeted Genome Editing I|Volume 24, SUPPLEMENT 1, S50, May 1, 2016, and WO2018106693A1.

### Small Molecules

In some embodiments a polypeptide described herein (e.g., a gene modifying polypeptide) is controllable via a small molecule. In some embodiments, the polypeptide is dimerized via a small molecule.

In some embodiment, the polypeptide is controllable via Chemical Induction of Dimerization (CID) with small molecules. CID is generally used to generate switches of protein function to alter cell physiology. An exemplary high specificity, efficient dimerizer is rimiducid (AP1903), which has two identical, protein-binding surfaces arranged tail-to-tail, each with high affinity and specificity for a mutant of FKBP12: FKBP12(F36V) (FKBP12v36, F_{V36} or Fᵥ), Attachment of one or more F_{V} domains onto one or more cell signaling molecules that normally rely on homodimerization can convert that protein to rimiducid control. Homodimerization with rimiducid is used in the context of an inducible caspase safety switch. This molecular switch that is controlled by a distinct dimerizer ligand, based on the heterodimerizing small molecule, rapamycin, or rapamycin analogs (“rapalogs”). Rapamycin binds to FKBP12, and its variants, and can induce heterodimerization of signaling domains that are fused to FKBP12 by binding to both FKBP12 and to polypeptides that contain the FKBP-rapamycin-binding (FRB) domain of mTOR. Provided in some embodiments of the present application are molecular switches that greatly augment the use of rapamycin, rapalogs and rimiducid as agents for therapeutic applications.

In some embodiments of the dual switch technology, a homodimerizer, such as AP1903 (rimiducid), directly induces dimerization or multimerization of polypeptides comprising an FKBP12 multimerizing region. In other embodiments, a polypeptide comprising an FKBP12 multimerization is multimerized, or aggregated by binding to a heterodimerizer, such as rapamycin or a rapalog, which also binds to an FRB or FRB variant multimerizing region on a chimeric polypeptide, also expressed in the modified cell, such as, for example, a chimeric antigen receptor. Rapamycin is a natural product macrolide that binds with high affinity (<1 nM) to FKBP12 and together initiates the high-affinity, inhibitory interaction with the FKBP-Rapamycin-Binding (FRB) domain of mTOR. FRB is small (89 amino acids) and can thereby be used as a protein “tag” or “handle” when appended to many proteins. Coexpression of a FRB-fused protein with a FKBP12-fused protein renders their approximation rapamycin-inducible (12-16). This can serve as the basis for a cell safety switch regulated by the orally available ligand, rapamycin, or derivatives of rapamycin (rapalogs) that do not inhibit mTOR at a low, therapeutic dose but instead bind with selected, Caspase-9-fused mutant FRB domains. (see Sabatini D M, et al., Cell. 1994; 78(1):35-43; Brown E J, et al., Nature. 1994; 369(6483):756-8; Chen J, et al., Proc Natl Acad Sci USA. 1995; 92(11):4947-51; and Choi J, Science. 1996; 273(5272):239-42).

In some embodiments, two levels of control are provided in the therapeutic cells. In embodiments, the first level of control may be tunable, i.e., the level of removal of the therapeutic cells may be controlled so that it results in partial removal of the therapeutic cells. In some embodiments, the chimeric antigen polypeptide comprises a binding site for rapamycin, or a rapamycin analog. In embodiments, also present in the therapeutic cell is a suicide gene, such as, for example, one encoding a caspase polypeptide. Using this controllable first level, the need for continued therapy may, in some embodiments, be balanced with the need to eliminate or reduce the level of negative side effects. In some embodiments, a rapamycin analog, a rapalog is administered to the patient, which then binds to both the caspase polypeptide and the chimeric antigen receptor, thus recruiting the caspase polypeptide to the location of the CAR, and aggregating the caspase polypeptide. Upon aggregation, the caspase polypeptide induces apoptosis. The amount of rapamycin or rapamycin analog administered to the patient may vary; if the removal of a lower level of cells by apoptosis is desired in order to reduce side effects and continue CAR therapy, a lower level of rapamycin or rapamycin may be administered to the patient. In some embodiments, the second level of control may be designed to achieve the maximum level of cell elimination. This second level may be based, for example, on the use of rimiducid, or AP1903. If there is a need to rapidly eliminate up to 100% of the therapeutic cells, the AP1903 may be administered to the patient. The multimeric AP1903 binds to the caspase polypeptide, leading to multimerization of the caspase polypeptide and apoptosis. In certain examples, second level may also be tunable, or controlled, by the level of AP1903 administered to the subject.

In certain embodiments, small molecules can be used to control genes, as described in for example, U.S. Ser. No. 10/584,351 at 47:53-56:47 (incorporated by reference herein in its entirety), together suitable ligands for the control features, e.g., in U.S. Ser. No. 10/584,351 at 56:48, et seq. as well as U10046049 at 43:27-52:20, incorporated by reference as well as the description of ligands for such control systems at 52:21, et seq.

### Chemically Modified Nucleic Acids and Nucleic Acid End Features

A nucleic acid described herein (e.g., a template nucleic acid, e.g., a template RNA; or a nucleic acid (e.g., mRNA) encoding a gene modifying polypeptide) can comprise unmodified or modified nucleobases. Naturally occurring RNAs are synthesized from four basic ribonucleotides: ATP, CTP, UTP and GTP, but may contain post-transcriptionally modified nucleotides. Further, approximately one hundred different nucleoside modifications have been identified in RNA (Rozenski, J, Crain, P, and McCloskey, J. (1999). The RNA Modification Database: 1999 update. Nucl Acids Res 27: 196-197). An RNA can also comprise wholly synthetic nucleotides that do not occur in nature.

In some embodiments, the chemically modification is one provided in PCT/US2016/032454, US Pat. Pub. No. 20090286852, of International Application No. WO/2012/019168, WO/2012/045075, WO/2012/135805, WO/2012/158736, WO/2013/039857, WO/2013/039861, WO/2013/052523, WO/2013/090648, WO/2013/096709, WO/2013/101690, WO/2013/106496, WO/2013/130161, WO/2013/151669, WO/2013/151736, WO/2013/151672, WO/2013/151664, WO/2013/151665, WO/2013/151668, WO/2013/151671, WO/2013/151667, WO/2013/151670, WO/2013/151666, WO/2013/151663, WO/2014/028429, WO/2014/081507, WO/2014/093924, WO/2014/093574, WO/2014/113089, WO/2014/144711, WO/2014/144767, WO/2014/144039, WO/2014/152540, WO/2014/152030, WO/2014/152031, WO/2014/152027, WO/2014/152211, WO/2014/158795, WO/2014/159813, WO/2014/164253, WO/2015/006747, WO/2015/034928, WO/2015/034925, WO/2015/038892, WO/2015/048744, WO/2015/051214, WO/2015/051173, WO/2015/051169, WO/2015/058069, WO/2015/085318, WO/2015/089511, WO/2015/105926, WO/2015/164674, WO/2015/196130, WO/2015/196128, WO/2015/196118, WO/2016/011226, WO/2016/011222, WO/2016/011306, WO/2016/014846, WO/2016/022914, WO/2016/036902, WO/2016/077125, or WO/2016/077123, each of which is herein incorporated by reference in its entirety. It is understood that incorporation of a chemically modified nucleotide into a polynucleotide can result in the modification being incorporated into a nucleobase, the backbone, or both, depending on the location of the modification in the nucleotide. In some embodiments, the backbone modification is one provided in EP 2813570, which is herein incorporated by reference in its entirety. In some embodiments, the modified cap is one provided in US Pat. Pub. No. 20050287539, which is herein incorporated by reference in its entirety.

In some embodiments, the chemically modified nucleic acid (e.g., RNA, e.g., mRNA) comprises one or more of ARCA: anti-reverse cap analog (m27.3′-OGP3G), GP3G (Unmethylated Cap Analog), m7GP3G (Monomethylated Cap Analog), m32.2.7GP3G (Trimethylated Cap Analog), m5CTP (5′-methyl-cytidine triphosphate), m6ATP (N6-methyl-adenosine-5′-triphosphate), s2UTP (2-thio-uridine triphosphate), and Ψ (pseudouridine triphosphate).

In some embodiments, the chemically modified nucleic acid comprises a 5′ cap, e.g.: a 7-methylguanosine cap (e.g., a 0-Me-m7G cap); a hypermethylated cap analog; an NAD+-derived cap analog (e.g., as described in Kiledjian, Trends in Cell Biology 28, 454-464 (2018)); or a modified, e.g., biotinylated, cap analog (e.g., as described in Bednarek et al., Phil Trans R Soc B 373, 20180167 (2018)).

In some embodiments, the chemically modified nucleic acid comprises a 3′ feature selected from one or more of: a polyA tail; a 16-nucleotide long stem-loop structure flanked by unpaired 5 nucleotides (e.g., as described by Mannironi et al., Nucleic Acid Research 17, 9113-9126 (1989)); a triple-helical structure (e.g., as described by Brown et al., PNAS 109, 19202-19207 (2012)); a tRNA, Y RNA, or vault RNA structure (e.g., as described by Labno et al., Biochemica et Biophysica Acta 1863, 3125-3147 (2016)); incorporation of one or more deoxyribonucleotide triphosphates (dNTPs), 2′O-Methylated NTPs, or phosphorothioate-NTPs; a single nucleotide chemical modification (e.g., oxidation of the 3′ terminal ribose to a reactive aldehyde followed by conjugation of the aldehyde-reactive modified nucleotide); or chemical ligation to another nucleic acid molecule.

In some embodiments, the nucleic acid (e.g., template nucleic acid or nucleic acid encoding the gene modifying polypeptide) comprises one or more modified nucleotides, e.g., selected from dihydrouridine, inosine, 7-methylguanosine, 5-methylcytidine (5mC), 5′ Phosphate ribothymidine, 2′-O-methyl ribothymidine, 2′-O-ethyl ribothymidine, 2′-fluoro ribothymidine, C-5 propynyl-deoxycytidine (pdC), C-5 propynyl-deoxyuridine (pdU), C-5 propynyl-cytidine (pC), C-5 propynyl-uridine (pU), 5-methyl cytidine, 5-methyl uridine, 5-methyl deoxycytidine, 5-methyl deoxyuridine methoxy, 2,6-diaminopurine, 5′-Dimethoxytrityl-N4-ethyl-2′-deoxycytidine, C-5 propynyl-f-cytidine (pfC), C-5 propynyl-f-uridine (pfU), 5-methyl f-cytidine, 5-methyl f-uridine, C-5 propynyl-m-cytidine (pmC), C-5 propynyl-f-uridine (pmU), 5-methyl m-cytidine, 5-methyl m-uridine, LNA (locked nucleic acid), MGB (minor groove binder) pseudouridine (Ψ), 1-N-methylpseudouridine (1-Me-Ψ), or 5-methoxyuridine (5-MO-U).

In some embodiments, the nucleic acid comprises a backbone modification, e.g., a modification to a sugar or phosphate group in the backbone. In some embodiments, the nucleic acid comprises a nucleobase modification.

In some embodiments, the nucleic acid comprises one or more chemically modified nucleotides of Table M1, one or more chemical backbone modifications of Table M2, one or more chemically modified caps of Table M3. For instance, in some embodiments, the nucleic acid comprises two or more (e.g., 3, 4, 5, 6, 7, 8, 9, or 10 or more) different types of chemical modifications. As an example, the nucleic acid may comprise two or more (e.g., 3, 4, 5, 6, 7, 8, 9, or 10 or more) different types of modified nucleobases, e.g., as described herein, e.g., in Table M1. Alternatively or in combination, the nucleic acid may comprise two or more (e.g., 3, 4, 5, 6, 7, 8, 9, or 10 or more) different types of backbone modifications, e.g., as described herein, e.g., in Table M2. Alternatively or in combination, the nucleic acid may comprise one or more modified cap, e.g., as described herein, e.g., in Table M3. For instance, in some embodiments, the nucleic acid comprises one or more type of modified nucleobase and one or more type of backbone modification; one or more type of modified nucleobase and one or more modified cap; one or more type of modified cap and one or more type of backbone modification; or one or more type of modified nucleobase, one or more type of backbone modification, and one or more type of modified cap.

In some embodiments, the nucleic acid comprises one or more (e.g., 2, 3, 4, 5, 6, 7, 8, 9, 10, 20, 30, 40, 50, 60, 70, 80, 90, 100, 150, 200, 250, 300, 350, 400, 450, 500, 600, 700, 800, 900, 1000, or more) modified nucleobases. In some embodiments, all nucleobases of the nucleic acid are modified. In some embodiments, the nucleic acid is modified at one or more (e.g., 2, 3, 4, 5, 6, 7, 8, 9, 10, 20, 30, 40, 50, 60, 70, 80, 90, 100, 150, 200, 250, 300, 350, 400, 450, 500, 600, 700, 800, 900, 1000, or more) positions in the backbone. In some embodiments, all backbone positions of the nucleic acid are modified.

TABLE M1
Modified nucleotides

| | |
|---|---|
| 5-aza-uridine | N2-methyl-6-thio-guanosine |
| 2-thio-5-aza-midine | N2,N2-dimethyl-6-thio-guanosine |
| 2-thiouridine | pyridin-4-one ribonucleoside |
| 4-thio-pseudouridine | 2-thio-5-aza-uridine |
| 2-thio-pseudouridine | 2-thiomidine |
| 5-hydroxyuridine | 4-thio-pseudomidine |
| 3-methyluridine | 2-thio-pseudowidine |
| 5-carboxymethyl-uridine | 3-methylmidine |
| 1-carboxymethyl-pseudouridine | 1-propynyl-pseudomidine |
| 5-propynyl-uridine | 1-methyl-1-deaza-pseudomidine |
| 1-propynyl-pseudouridine | 2-thio-1-methyl-1-deaza-pseudouridine |
| 5-taurinomethyluridine | 4-methoxy-pseudomidine |
| 1-taurinomethyl-pseudouridine | 5′-O-(1-Thiophosphate)-Adenosine |
| 5-taurinomethyl-2-thio-uridine | 5′-O-(1-Thiophosphate)-Cytidine |
| 1-taurinomethyl-4-thio-uridine | 5′-O-(1-thiophosphate)-Guanosine |
| 5-methyl-uridine | 5′-O-(1-Thiophophate)-Uridine |
| 1-methyl-pseudouridine | 5′-O-(1-Thiophosphate)-Pseudouridine |
| 4-thio-1-methyl-pseudouridine | 2′-O-methyl-Adenosine |
| 2-thio-1-methyl-pseudouridine | 2′-O-methyl-Cytidine |
| 1-methyl-1-deaza-pseudouridine | 2′-O-methyl-Guanosine |
| 2-thio-1-methyl-1-deaza-pseudomidine | 2′-O-methyl-Uridine |
| dihydrouridine | 2′-O-methyl-Pseudouridine |
| dihydropseudouridine | 2′-O-methyl-Inosine |
| 2-thio-dihydromidine | 2-methyladenosine |
| 2-thio-dihydropseudouridine | 2-methylthio-N6-methyladenosine |
| 2-methoxyuridine | 2-methylthio-N6 isopentenyladenosine |
| 2-methoxy-4-thio-uridine | 2-methylthio-N6-(cis- |
| 4-methoxy-pseudouridine | hydroxyisopentenyl)adenosine |
| 4-methoxy-2-thio-pseudouridine | N6-methyl-N6-threonylcarbamoyladenosine |
| 5-aza-cytidine | N6-hydroxynorvalylcarbamoyladenosine |
| pseudoisocytidine | 2-methylthio-N6-hydroxynorvalyl |
| 3-methyl-cytidine | carbamoyladenosine |
| N4-acetylcytidine | 2′-O-ribosyladenosine (phosphate) |
| 5-formylcytidine | 1,2′-O-dimethylinosine |
| N4-methylcytidine | 5,2′-O-dimethylcytidine |
| 5-hydroxymethylcytidine | N4-acetyl-2′-O-methylcytidine |
| 1-methyl-pseudoisocytidine | Lysidine |
| pyrrolo-cytidine | 7-methylguanosine |
| pyrrolo-pseudoisocytidine | N2,2′-O-dimethylguanosine |
| 2-thio-cytidine | N2,N2,2′-O-trimethylguanosine |
| 2-thio-5-methyl-cytidine | 2′-O-ribosylguanosine (phosphate) |
| 4-thio-pseudoisocytidine | Wybutosine |
| 4-thio-1-methyl-pseudoisocytidine | Peroxywybutosine |
| 4-thio-1-methyl-1-deaza-pseudoisocytidine | Hydroxywybutosine |
| 1-methyl-1-deaza-pseudoisocytidine | undermodified hydroxywybutosine |
| zebularine | methylwyosine |
| 5-aza-zebularine | queuosine |
| 5-methyl-zebularine | epoxyqueuosine |
| 5-aza-2-thio-zebularine | galactosyl-queuosine |
| 2-thio-zebularine | mannosyl-queuosine |
| 2-methoxy-cytidine | 7-cyano-7-deazaguanosine |
| 2-methoxy-5-methyl-cytidine | 7-aminomethyl-7-deazaguanosine |
| 4-methoxy-pseudoisocytidine | archaeosine |
| 4-methoxy-1-methyl-pseudoisocytidine | 5,2′-O-dimethyluridine |
| 2-aminopurine | 4-thiouridine |
| 2,6-diaminopurine | 5-methyl-2-thiouridine |
| 7-deaza-adenine | 2-thio-2′-O-methyluridine |
| 7-deaza-8-aza-adenine | 3-(3-amino-3-carboxypropyl)uridine |
| 7-deaza-2-aminopurine | 5-methoxyuridine |
| 7-deaza-8-aza-2-aminopurine | uridine 5-oxyacetic acid |
| 7-deaza-2,6- diaminopurine | uridine 5-oxyacetic acid methyl ester |
| 7-deaza-8-aza-2,6-diarninopurine | 5-(carboxyhydroxymethyl)uridine) |
| 1-methyladenosine | 5-(carboxyhydroxymethyl)uridine methyl ester |
| N6-isopentenyladenosine | 5-methoxycarbonylmethyluridine |
| N6-(cis-hydroxyisopentenyl)adenosine | 5-methoxycarbonylmethyl-2′-O-methyluridine |
| 2-methylthio-N6-(cis-hydroxyisopentenyl) | 5-methoxycarbonylmethyl-2-thiouridine |
| adenosine | 5-aminomethyl-2-thiouridine |
| N6-glycinylcarbamoyladenosine | 5-methylaminomethyluridine |
| N6-threonylcarbamoyladenosine | 5-methylaminomethyl-2-thiouridine |
| 2-methylthio-N6-threonyl | 5-methylaminomethyl-2-selenouridine |
| carbamoyladenosine | 5-carbamoylmethyluridine |
| N6,N6-dimethyladenosine | 5-carbamoylmethyl-2′-O-methyluridine |
| 7-methyladenine | 5-carboxymethylaminomethyluridine |
| 2-methylthio-adenine | 5-carboxymethylaminomethyl-2′-O- |
| 2-methoxy-adenine | methyluridine |
| inosine | 5-carboxymethylaminomethyl-2-thiouridine |
| 1-methyl-inosine | N4,2′-O-dimethylcytidine |
| wyosine | 5-carboxymethyluridine |
| wybutosine | N6,2′-O-dimethyladenosine |
| 7-deaza-guanosine | N,N6,O-2′-trimethyladenosine |
| 7-deaza-8-aza-guanosine | N2,7-dimethylguanosine |
| 6-thio-guanosine | N2,N2,7-trimethylguanosine |
| 6-thio-7-deaza-guanosine | 3,2′-O-dimethyluridine |
| 6-thio-7-deaza-8-aza-guanosine | 5-methyldihydrouridine |
| 7-methyl-guanosine | 5-formyl-2′-O-methylcytidine |
| 6-thio-7-methyl-guanosine | 1,2′-O-dimethylguanosine |
| 7-methylinosine | 4-demethylwyosine |
| 6-methoxy-guanosine | Isowyosine |
| 1-methylguanosine | N6-acetyladenosine |
| N2-methylguanosine | |
| N2,N2-dimethylguanosine | |
| 8-oxo-guanosine | |
| 7-methyl-8-oxo-guanosine | |
| 1-methyl-6-thio-guanosine | |

TABLE M2
Backbone modifications

2′-O-Methyl backbone
Peptide Nucleic Acid (PNA) backbone
phosphorothioate backbone
morpholino backbone
carbamate backbone
siloxane backbone
sulfide backbone
sulfoxide backbone
sulfone backbone
formacetyl backbone
thioformacetyl backbone
methyleneformacetyl backbone
riboacetyl backbone
alkene containing backbone
sulfamate backbone
sulfonate backbone
sulfonamide backbone
methyleneimino backbone
methylenehydrazino backbone
amide backbone

TABLE M3
Modified caps

m7GpppA
m7GpppC
m2,7GpppG
m2,2,7GpppG
m7Gpppm7G
m7,2′OmeGpppG
m72′dGpppG
m7,3′OmeGpppG
m7,3′dGpppG
GppppG
m7GppppG
m7GppppA
m7GppppC
m2,7GppppG
m2,2,7GppppG
m7Gppppm7G
m7,2′OmeGppppG
m72′dGppppG
m7,3′OmeGppppG
m7,3′dGppppG

### Combined Systems for Installing Multiple Gene Modifications

Combined Systems for Installing Two Genes within a Cell

As described herein, a first gene modifying system, used to insert a heterologous object sequence (e.g., a CAR) into the genome of a cell, may be combined with a second gene modifying system for installing a second heterologous object sequence (e.g., a second CAR) into the genome of a cell. Consequently, in some aspects, the present disclosure provides a first gene modifying system and a second gene modifying system. The first gene modifying system comprises a first retrotransposon gene modifying polypeptide and a first template RNA (e.g., comprising a heterologous object sequence encoding a CAR). The second gene modifying system comprises a second retrotransposon gene modifying polypeptide and a second template RNA.

In some embodiments, two systems may be utilized to introduce multiple edits to the same cell. In some embodiments, the two systems may be introduced into cells simultaneously or separately. In some embodiments, editing efficiency when inserting whole genes using the first gene modifying system is not affected by the co-delivery of another gene modifying system to insert another gene into the cell. In some embodiments, treating T cells with both the first and second systems results in co-occurrence of the two editing events in the same cell.

In some embodiments, a gene modifying system comprising a retrotransposon gene modifying polypeptide and a first template RNA, used to insert a heterologous object sequence (e.g., a CAR) into the genome of a cell, may be combined with a second template RNA for installing a second heterologous object sequence (e.g., a second CAR) into the genome of a cell.

In some embodiments, the system comprising two template RNAs may be utilized to introduce multiple edits to the same cell. In some embodiments, the two template RNAs may be introduced into cells simultaneously or separately. In some embodiments, editing efficiency when inserting whole genes using the system with the first template RNA is not affected by the co-delivery of another template RNA to insert another gene into the cell. In some embodiments, treating T cells with the system comprising two template RNAs results in co-occurrence of the two editing events in the same cell.

In some embodiments, the two systems or system comprising two template RNAs may be used to insert two CARs specific to different ligands on a target cell (e.g., a neoplastic cell). For example, the system or systems described herein could insert two CARs having specificity to CD20 and CD22, CD19 and CD20, CD19 and CD22, or such as BCMA and GPRC5D.

In other embodiments, the two systems or system comprising two template RNAs may be used to insert a CAR molecule and a TCR or safety/suicide switch (e.g., a caspase (e.g., caspase-9 or iCasp-9) or RQR8) into a cell.

Combined Systems for Installing a Gene and Editing a Gene within a Cell

In another aspect, a first gene modifying system, used to insert a heterologous object sequence (e.g., a CAR) into the genome of a cell, may be combined with an additional system for modifying DNA to install a mutation (e.g., resulting in knock down or knock out of a target gene, or a correction or deletion of an aberrant gene) into a human gene.

In some embodiments, two systems may be utilized to introduce multiple edits to the same cell. In some embodiments, the two systems may be introduced into cells simultaneously or separately. In some embodiments, editing efficiency when inserting whole genes using the first gene modifying system is not affected by the co-delivery of another exemplary system containing a heterologous gene modifying polypeptide (e.g., directing a short edit, e.g., an indel or substitution in the DNA). In some embodiments, editing efficiency when making short edits (e.g., editing of 5 nucleotides or fewer) is not affected by the co-delivery of a gene modifying system containing a retrotransposon gene modifying polypeptide (e.g., directing the insertion of a gene). In some embodiments, treating T cells with both the first and second systems results in high level of co-occurrence of the two editing events in the same cell. In some embodiments, no skewing in the distribution of the subpopulations or phenotypes of T cells is observed when comparing mock treated T cells, T cells edited by just the first gene modifying system, and T cells edited by both the first and second gene modifying systems.

In some embodiments, the additional system comprises: (A) a heterologous gene modifying polypeptide or a nucleic acid encoding the heterologous gene modifying polypeptide, wherein the heterologous gene modifying polypeptide comprises (i) a reverse transcriptase domain, and either (x) an endonuclease domain that contains DNA binding functionality or (y) an endonuclease domain and separate DNA binding domain; and (B) a template RNA. A heterologous gene modifying polypeptide, in some embodiments, acts as a substantially autonomous protein machine capable of integrating a template nucleic acid sequence into a target DNA molecule (e.g., in a mammalian host cell, such as a genomic DNA molecule in the host cell), substantially without relying on host machinery. For example, the heterologous gene modifying protein may comprise a DNA-binding domain, a reverse transcriptase domain, and an endonuclease domain. In some embodiments, the DNA-binding function may involve an RNA component that directs the protein to a DNA sequence, e.g., a gRNA spacer. In other embodiments, the heterologous gene modifying polypeptide may comprise a reverse transcriptase domain and an endonuclease domain. The RNA template element of a system is typically heterologous to the heterologous gene modifying polypeptide element and provides an object sequence to be inserted (reverse transcribed) into the host genome. In some embodiments, the heterologous gene modifying polypeptide is capable of target primed reverse transcription. In some embodiments, the heterologous gene modifying polypeptide is capable of second-strand synthesis.

In some embodiments the system is combined with a second polypeptide. In some embodiments, the second polypeptide may comprise an endonuclease domain. In some embodiments, the second polypeptide may comprise a polymerase domain, e.g., a reverse transcriptase domain. In some embodiments, the second polypeptide may comprise a DNA-dependent DNA polymerase domain. In some embodiments, the second polypeptide aids in completion of the genome edit, e.g., by contributing to second-strand synthesis or DNA repair resolution.

A functional heterologous gene modifying polypeptide can be made up of unrelated DNA binding, reverse transcription, and endonuclease domains. This modular structure allows combining of functional domains, e.g., dCas9 (DNA binding), MMLV reverse transcriptase (reverse transcription), FokI (endonuclease). In some embodiments, multiple functional domains may arise from a single protein, e.g., Cas9 or Cas9 nickase (DNA binding, endonuclease).

In some embodiments, a heterologous gene modifying polypeptide includes one or more domains that, collectively, facilitate 1) binding the template nucleic acid, 2) binding the target DNA molecule, and 3) facilitate integration of the at least a portion of the template nucleic acid into the target DNA. In some embodiments, the heterologous gene modifying polypeptide is an engineered polypeptide that comprises one or more amino acid substitutions to a corresponding naturally occurring sequence. In some embodiments, the heterologous gene modifying polypeptide comprises two or more domains that are heterologous relative to each other, e.g., through a heterologous fusion (or other conjugate) of otherwise wild-type domains, or well as fusions of modified domains, e.g., by way of replacement or fusion of a heterologous sub-domain or other substituted domain. For instance, in some embodiments, one or more of: the RT domain is heterologous to the DBD; the DBD is heterologous to the endonuclease domain; or the RT domain is heterologous to the endonuclease domain.

In some embodiments, a template RNA molecule for use in the system comprises, from 5′ to 3′ (1) a gRNA spacer; (2) a gRNA scaffold; (3) heterologous object sequence (4) a primer binding site (PBS) sequence. In some embodiments:
(1) Is a gRNA spacer of ˜18-22 nt, e.g., is 20 nt
(2) Is a gRNA scaffold comprising one or more hairpin loops, e.g., 1, 2, of 3 loops for associating the template with a Cas domain, e.g., a nickase Cas9 domain. In some embodiments, the gRNA scaffold comprises the sequence, from 5′ to 3′, GTTTTAGAGCTAGAAATAGCAAGTTAAAATAAGGCTAGTCCGTTATCAACTT GAAAAAGTGGGACCGAGTCGGTCC (SEQ ID NO: 5008).
(3) In some embodiments, the heterologous object sequence is, e.g., 7-74, e.g., 10-20, 20-30, 30-40, 40-50, 50-60, 60-70, or 70-80 nt or, 80-90 nt in length. In some embodiments, the first (most 5′) base of the sequence is not C.
(4) In some embodiments, the PBS sequence that binds the target priming sequence after nicking occurs is e.g., 3-20 nt, e.g., 7-15 nt, e.g., 12-14 nt. In some embodiments, the PBS sequence has 40-60% GC content.

In some embodiments, a second gRNA associated with the system may help drive complete integration. In some embodiments, the second gRNA may target a location that is 0-200 nt away from the first-strand nick, e.g., 0-50, 50-100, 100-200 nt away from the first-strand nick. In some embodiments, the second gRNA can only bind its target sequence after the edit is made, e.g., the gRNA binds a sequence present in the heterologous object sequence, but not in the initial target sequence.

In some embodiments, a heterologous gene modifying polypeptide as described herein comprises a reverse transcriptase or RT domain (e.g., as described herein) that comprises a MoMLV RT sequence or variant thereof. In embodiments, the MoMLV RT sequence comprises one or more mutations selected from D200N, L603W, T330P, T306K, W313F, D524G, E562Q, D583N, P51L, S67R, E67K, T197A, H204R, E302K, F309N, L435G, N454K, H594Q, D653N, R110S, and K103L. In embodiments, the MoMLV RT sequence comprises a combination of mutations, such as D200N, L603W, and T330P, optionally further including T306K and/or W313F.

In some embodiments, an endonuclease domain (e.g., as described herein) nCas9, e.g., comprising an N863A mutation (e.g., in spCas9) or a H840A mutation.

In some embodiments, the heterologous object sequence (e.g., of a system as described herein) is about 1-50, 50-100, 100-200, 200-300, 300-400, 400-500, 500-600, 600-700, 700-800, 800-900, 900-1000, or more, nucleotides in length.

In some embodiments, the RT and endonuclease domains are joined by a flexible linker, e.g., comprising the amino acid sequence SGGSSGGSSGSETPGTSESATPESSGGSSGGSS (SEQ ID NO: 5006).

In some embodiments, the endonuclease domain is N-terminal relative to the RT domain. In some embodiments, the endonuclease domain is C-terminal relative to the RT domain.

In some embodiments, the system incorporates a heterologous object sequence into a target site by TPRT, e.g., as described herein.

In some embodiments, a heterologous gene modifying polypeptide comprises a DNA binding domain. In some embodiments, a heterologous gene modifying polypeptide comprises an RNA binding domain. In some embodiments, the RNA binding domain comprises an RNA binding domain of B-box protein, MS2 coat protein, dCas, or an element of a sequence of a table herein. In some embodiments, the RNA binding domain is capable of binding to a template RNA with greater affinity than a reference RNA binding domain.

In some embodiments, a system comprising a heterologous gene modifying polypeptide is capable of producing an insertion into the target site of at least 1, 2, 3, 4, or 5 nucleotides (and optionally no more than 5 nucleotides). In some embodiments, a gene modifying system is capable of producing one or more substitutions, e.g., at least 1, 2, 3, 4, or 5 substitutions (and optionally no more than 5 nucleotides). In some embodiments, system comprising a heterologous gene modifying polypeptide is capable of producing a deletion of at least 1, 2, 3, 4, or 5 nucleotides (and optionally no more than 5 nucleotides). In some embodiments, the substitution is a transition mutation. In some embodiments, the substitution is a transversion mutation. In some embodiments, the substitution converts an adenine to a thymine, an adenine to a guanine, an adenine to a cytosine, a guanine to a thymine, a guanine to a cytosine, a guanine to an adenine, a thymine to a cytosine, a thymine to an adenine, a thymine to a guanine, a cytosine to an adenine, a cytosine to a guanine, or a cytosine to a thymine.

In some embodiments, an insertion, deletion, substitution, or combination thereof, increases or decreases expression (e.g. transcription or translation) of a gene. In some embodiments, an insertion, deletion, substitution, or combination thereof, increases or decreases expression (e.g. transcription or translation) of a gene by altering, adding, or deleting sequences in a promoter or enhancer, e.g. sequences that bind transcription factors. In some embodiments, an insertion, deletion, substitution, or combination thereof alters translation of a gene (e.g. alters an amino acid sequence), inserts or deletes a start or stop codon, alters or fixes the translation frame of a gene. In some embodiments, an insertion, deletion, substitution, or combination thereof alters splicing of a gene, e.g. by inserting, deleting, or altering a splice acceptor or donor site. In some embodiments, an insertion, deletion, substitution, or combination thereof alters transcript or protein half-life. In some embodiments, an insertion, deletion, substitution, or combination thereof alters protein localization in the cell (e.g. from the cytoplasm to a mitochondria, from the cytoplasm into the extracellular space (e.g. adds a secretion tag)). In some embodiments, an insertion, deletion, substitution, or combination thereof alters (e.g. improves) protein folding (e.g. to prevent accumulation of misfolded proteins). In some embodiments, an insertion, deletion, substitution, or combination thereof, alters, increases, decreases the activity of a gene, e.g. a protein encoded by the gene.

Exemplary heterologous gene modifying polypeptides, and systems comprising them and methods of using them are described, e.g., in PCT/US2021/020948, which is incorporated herein by reference with respect to retroviral RT domains, including the amino acid and nucleic acid sequences therein.

Exemplary heterologous gene modifying polypeptides and retroviral RT domain sequences are also described, e.g., in International Application No. PCT/US21/20948 filed Mar. 4, 2021, e.g., at Table 30, Table 31, and Table 44 therein; the entire application is incorporated by reference herein with respect to retroviral RTs, e.g., in said sequences and tables. Accordingly, a heterologous gene modifying polypeptide described herein may comprise an amino acid sequence according to any of the Tables mentioned in this paragraph, or a domain thereof (e.g., a retroviral RT domain), or a functional fragment or variant of any of the foregoing, or an amino acid sequence having at least 70%, 80%, 85%, 90%, 95%, or 99% identity thereto.

In some embodiments, a polypeptide for use in any of the systems described herein can be a molecular reconstruction or ancestral reconstruction based upon the aligned polypeptide sequence of multiple homologous proteins. In some embodiments, a reverse transcriptase domain for use in any of the systems described herein can be a molecular reconstruction or an ancestral reconstruction, or can be modified at particular residues, based upon alignments of reverse transcriptase domains from the same or different sources. A skilled artisan can, based on the Accession numbers provided herein, align polypeptides or nucleic acid sequences, e.g., by using routine sequence analysis tools as Basic Local Alignment Search Tool (BLAST) or CD-Search for conserved domain analysis. Molecular reconstructions can be created based upon sequence consensus, e.g. using approaches described in Ivics et al., Cell 1997, 501-510; Wagstaff et al., Molecular Biology and Evolution 2013, 88-99.

### Polypeptide Components of Heterologous Gene Modifying Systems

In some embodiments, the heterologous gene modifying polypeptide possesses the functions of DNA target site binding, template nucleic acid (e.g., RNA) binding, DNA target site cleavage, and template nucleic acid (e.g., RNA) writing, e.g., reverse transcription. In some embodiments, each functions is contained within a distinct domain. In some embodiments, a function may be attributed to two or more domains (e.g., two or more domains, together, exhibit the functionality). In some embodiments, two or more domains may have the same or similar function (e.g., two or more domains each independently have DNA-binding functionality, e.g., for two different DNA sequences). In other embodiments, one or more domains may be capable of enabling one or more functions, e.g., a Cas9 domain enabling both DNA binding and target site cleavage. In some embodiments, the domains are all located within a single polypeptide. In some embodiments, a first domain is in one polypeptide and a second domain is in a second polypeptide. For example, in some embodiments, the sequences may be split between a first polypeptide and a second polypeptide, e.g., wherein the first polypeptide comprises a reverse transcriptase (RT) domain and wherein the second polypeptide comprises a DNA-binding domain and an endonuclease domain, e.g., a nickase domain. As a further example, in some embodiments, the first polypeptide and the second polypeptide each comprise a DNA binding domain (e.g., a first DNA binding domain and a second DNA binding domain). In some embodiments, the first and second polypeptide may be brought together post-translationally via a split-intein to form a single heterologous gene modifying polypeptide.

In some aspects, a heterologous gene modifying polypeptide described herein comprises (e.g., a system described herein comprises a heterologous gene modifying polypeptide that comprises): 1) a Cas domain (e.g., a Cas nickase domain, e.g., a Cas9 nickase domain); 2) a reverse transcriptase (RT) domain disclosed herein, or a sequence having at least 70%, 75%, 80%, 85%, 90%, 95%, 97%, 98%, or 99% identity thereto, wherein the RT domain is C-terminal of the Cas domain; and a linker disposed between the RT domain and the Cas domain.

In some embodiments, the Cas domain comprises a sequence of Table 8A, or a sequence having at least 70%, 75%, 80%, 85%, 90%, 95%, 98%, or 99% identity thereto. In some embodiments, the heterologous gene modifying polypeptide comprises an amino acid sequence disclosed herein, or a sequence having at least 70%, 75%, 80%, 85%, 90%, 95%, 97%, 98%, or 99% identity thereto.

In some embodiments, the heterologous gene modifying polypeptide comprises a GG amino acid sequence between the Cas domain and the linker, an AG amino acid sequence between the RT domain and the second NLS, and/or a GG amino acid sequence between the linker and the RT domain. In some embodiments, the heterologous gene modifying polypeptide comprises a sequence of SEQ ID NO: 4000 which comprises the first NLS and the Cas domain, or a sequence having at least 70%, 75%, 80%, 85%, 90%, 95%, 98%, or 99% identity thereto. In some embodiments, the heterologous gene modifying polypeptide comprises a sequence of SEQ ID NO: 4001 which comprises the second NLS, or a sequence having at least 70%, 75%, 80%, 85%, 90%, 95%, 98%, or 99% identity thereto.

Exemplary N-terminal NLS-Cas9 domain
(SEQ ID NO: 4000)
MPAAKRVKLDGGDKKYSIGLDIGTNSVGWAVITDEYKVPSKKFKVLGNTDRHSIKKNLIGALLF
DSGETAEATRLKRTARRRYTRRKNRICYLQEIFSNEMAKVDDSFFHRLEESFLVEEDKKHERHP
IFGNIVDEVAYHEKYPTIYHLRKKLVDSTDKADLRLIYLALAHMIKFRGHFLIEGDLNPDNSDV
DKLFIQLVQTYNQLFEENPINASGVDAKAILSARLSKSRRLENLIAQLPGEKKNGLFGNLIALS
LGLTPNFKSNFDLAEDAKLQLSKDTYDDDLDNLLAQIGDQYADLFLAAKNLSDAILLSDILRVN
TEITKAPLSASMIKRYDEHHQDLTLLKALVRQQLPEKYKEIFFDQSKNGYAGYIDGGASQEEFY
KFIKPILEKMDGTEELLVKLNREDLLRKQRTFDNGSIPHQIHLGELHAILRRQEDFYPFLKDNR
EKIEKILTFRIPYYVGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDLLFKTNRKVTVKQLK
EDYFKKIECFDSVEISGVEDRFNASLGTYHDLLKIIKDKDFLDNEENEDILEDIVLTLTLFEDR
EMIEERLKTYAHLFDDKVMKQLKRRRYTGWGRLSRKLINGIRDKQSGKTILDFLKSDGFANRNF
MQLIHDDSLTFKEDIQKAQVSGQGDSLHEHIANLAGSPAIKKGILQTVKVVDELVKVMGRHKPE
NIVIEMARENQTTQKGQKNSRERMKRIEEGIKELGSQILKEHPVENTQLQNEKLYLYYLQNGRD
MYVDQELDINRLSDYDVDHIVPQSFLKDDSIDNKVLTRSDKARGKSDNVPSEEVVKKMKNYWRQ
LLNAKLITQRKFDNLTKAERGGLSELDKAGFIKRQLVETRQITKHVAQILDSRMNTKYDENDKL
IREVKVITLKSKLVSDFRKDFQFYKVREINNYHHAHDAYLNAVVGTALIKKYPKLESEFVYGDY
KVYDVRKMIAKSEQEIGKATAKYFFYSNIMNFFKTEITLANGEIRKRPLIETNGETGEIVWDKG
RDFATVRKVLSMPQVNIVKKTEVQTGGFSKESILPKRNSDKLIARKKDWDPKKYGGFDSPTVAY
SVLVVAKVEKGKSKKLKSVKELLGITIMERSSFEKNPIDFLEAKGYKEVKKDLIIKLPKYSLFE
LENGRKRMLASAGELQKGNELALPSKYVNFLYLASHYEKLKGSPEDNEQKQLFVEQHKHYLDEI
IEQISEFSKRVILADANLDKVLSAYNKHRDKPIREQAENIIHLFTLTNLGAPAAFKYFDTTIDR
KRYTSTKEVLDATLIHQSITGLYETRIDLSQLGGDGG
Exemplary C-terminal sequence comprising an NLS
(SEQ ID NO: 4001)
AGKRTADGSEFEKRTADGSEFESPKKKAKVE

### Writing Domain (RT Domain)

In certain aspects of the present invention, the writing domain of the heterologous gene modifying system possesses reverse transcriptase activity and is also referred to as a reverse transcriptase domain (a RT domain). In some embodiments, the RT domain comprises an RT catalytic portion and RNA-binding region (e.g., a region that binds the template RNA).

In some embodiments, a nucleic acid encoding the reverse transcriptase is altered from its natural sequence to have altered codon usage, e.g. improved for human cells. In some embodiments the reverse transcriptase domain is a heterologous reverse transcriptase from a retrovirus. In some embodiments, the RT domain has been mutated from its original amino acid sequence, e.g., has at least 1, 2, 3, 4, 5, 6, 7, 8, 9, 10, 20, 30, 40, 50, 60, 70, 80, 90, or 100 substitutions. In some embodiments, the RT domain is derived from the RT of a retrovirus, e.g., HIV-1 RT, Moloney Murine Leukemia Virus (MMLV) RT, avian myeloblastosis virus (AMV) RT, or Rous Sarcoma Virus (RSV) RT.

In some embodiments, the retroviral reverse transcriptase (RT) domain exhibits enhanced stringency of target-primed reverse transcription (TPRT) initiation, e.g., relative to an endogenous RT domain. In some embodiments, the RT domain initiates TPRT when the 3 nt in the target site immediately upstream of the first strand nick, e.g., the genomic DNA priming the RNA template, have at least 66% or 100% complementarity to the 3 nt of homology in the RNA template. In some embodiments, the RT domain initiates TPRT when there are less than 5 nt mismatched (e.g., less than 1, 2, 3, 4, or 5 nt mismatched) between the template RNA homology and the target DNA priming reverse transcription. In some embodiments, the RT domain is modified such that the stringency for mismatches in priming the TPRT reaction is increased, e.g., wherein the RT domain does not tolerate any mismatches or tolerates fewer mismatches in the priming region relative to a wild-type (e.g., unmodified) RT domain. In some embodiments, the RT domain comprises a HIV-1 RT domain. In embodiments, the HIV-1 RT domain initiates lower levels of synthesis even with three nucleotide mismatches relative to an alternative RT domain (e.g., as described by Jamburuthugoda and Eickbush J Mol Biol 407(5):661-672 (2011); incorporated herein by reference in its entirety). In some embodiments, the RT domain forms a dimer (e.g., a heterodimer or homodimer). In some embodiments, the RT domain is monomeric. In some embodiments, an RT domain, naturally functions as a monomer or as a dimer (e.g., heterodimer or homodimer). In some embodiments, an RT domain naturally functions as a monomer, e.g., is derived from a virus wherein it functions as a monomer. In embodiments, the RT domain is selected from an RT domain from murine leukemia virus (MLV; sometimes referred to as MoMLV) (e.g., P03355), porcine endogenous retrovirus (PERV) (e.g., UniProt Q4VFZ2), mouse mammary tumor virus (MMTV) (e.g., UniProt P03365), Avian reticuloendotheliosis virus (AVIRE) (e.g., UniProtKB accession: P03360); Feline leukemia virus (FLV or FeLV) (e.g., e.g., UniProtKB accession: P10273); Mason-Pfizer monkey virus (MPMV) (e.g., UniProt P07572), bovine leukemia virus (BLV) (e.g., UniProt P03361), human T-cell leukemia virus-1 (HTLV-1) (e.g., UniProt P03362), human foamy virus (HFV) (e.g., UniProt P14350), simian foamy virus (SFV) (e.g., SFV3L) (e.g., UniProt P23074 or P27401), or bovine foamy/syncytial virus (BFV/BSV) (e.g., UniProt 041894), or a functional fragment or variant thereof (e.g., an amino acid sequence having at least 70%, 80%, 90%, 95%, or 99% identity thereto). In some embodiments, an RT domain is dimeric in its natural functioning. In some embodiments, the RT domain is derived from a virus wherein it functions as a dimer. In embodiments, the RT domain is selected from an RT domain from avian sarcoma/leukemia virus (ASLV) (e.g., UniProt A0A142BKH1), Rous sarcoma virus (RSV) (e.g., UniProt P03354), avian myeloblastosis virus (AMV) (e.g., UniProt Q83133), human immunodeficiency virus type I (HIV-1) (e.g., UniProt P03369), human immunodeficiency virus type II (HIV-2) (e.g., UniProt P15833), simian immunodeficiency virus (SIV) (e.g., UniProt P05896), bovine immunodeficiency virus (BIV) (e.g., UniProt P19560), equine infectious anemia virus (EIAV) (e.g., UniProt P03371), or feline immunodeficiency virus (FIV) (e.g., UniProt P16088) (Herschhorn and Hizi Cell Mol Life Sci 67(16):2717-2747 (2010)), or a functional fragment or variant thereof (e.g., an amino acid sequence having at least 70%, 80%, 90%, 95%, or 99% identity thereto). Naturally heterodimeric RT domains may, in some embodiments, also be functional as homodimers. In some embodiments, dimeric RT domains are expressed as fusion proteins, e.g., as homodimeric fusion proteins or heterodimeric fusion proteins. In some embodiments, the RT function of the system is fulfilled by multiple RT domains (e.g., as described herein). In further embodiments, the multiple RT domains are fused or separate, e.g., may be on the same polypeptide or on different polypeptides.

In some embodiments, a system described herein comprises an integrase domain, e.g., wherein the integrase domain may be part of the RT domain. In some embodiments, an RT domain (e.g., as described herein) comprises an integrase domain. In some embodiments, an RT domain (e.g., as described herein) lacks an integrase domain, or comprises an integrase domain that has been inactivated by mutation or deleted. In some embodiment, a system described herein comprises an RNase H domain, e.g., wherein the RNase H domain may be part of the RT domain. In some embodiments, the RNase H domain is not part of the RT domain and is covalently linked via a flexible linker. In some embodiments, an RT domain (e.g., as described herein) comprises an RNase H domain, e.g., an endogenous RNAse H domain or a heterologous RNase H domain. In some embodiments, an RT domain (e.g., as described herein) lacks an RNase H domain. In some embodiments, an RT domain (e.g., as described herein) comprises an RNase H domain that has been added, deleted, mutated, or swapped for a heterologous RNase H domain. In some embodiments, the polypeptide comprises an inactivated endogenous RNase H domain. In some embodiments, an endogenous RNase H domain from one of the other domains of the polypeptide is genetically removed such that it is not included in the polypeptide, e.g., the endogenous RNase H domain is partially or completely truncated from the comprising domain. In some embodiments, mutation of an RNase H domain yields a polypeptide exhibiting lower RNase activity, e.g., as determined by the methods described in Kotewicz et al. Nucleic Acids Res 16(1):265-277 (1988) (incorporated herein by reference in its entirety), e.g., lower by at least 10%, 20%, 30%, 40%, 50%, 60%, 70%, 80%, or 90% compared to an otherwise similar domain without the mutation. In some embodiments, RNase H activity is abolished.

In some embodiments, an RT domain is mutated to increase fidelity compared to an otherwise similar domain without the mutation. For instance, in some embodiments, a YADD (SEQ ID NO: 15409) or YMDD motif (SEQ ID NO: 15410) in an RT domain (e.g., in a reverse transcriptase) is replaced with YVDD (SEQ ID NO: 15411). In embodiments, replacement of the YADD (SEQ ID NO: 15409) or YMDD (SEQ ID NO: 15410) or YVDD (SEQ ID NO: 15411) results in higher fidelity in retroviral reverse transcriptase activity (e.g., as described in Jamburuthugoda and Eickbush J Mol Biol 2011; incorporated herein by reference in its entirety).

In some embodiments, a gene modifying polypeptide described herein comprises an RT domain having an amino acid sequence according to Table 6, or a sequence having at least 70%, 80%, 85%, 90%, 95%, 97%, 98%, or 99% identity thereto. In some embodiments, a nucleic acid described herein encodes an RT domain having an amino acid sequence according to Table 6, or a sequence having at least 70%, 80%, 85%, 90%, 95%, 97%, 98%, or 99% identity thereto.

TABLE 6
Exemplary reverse transcriptase domains from retroviruses

| RT | SEQ ID | |
|---|---|---|
| Name | NO: | RT amino acid sequence |
| AVIRE_ | 8,003 | TAPLEEEYRLFLEAPIQNVTLLEQWKREIPKVWAEINPPGLASTQAPIHVQLLSTALPVRVRQYPITLEAKRSLRETIRKFRAAGILRPVHSPWNTPLLPV |
| P03360_ | | RKSGTSEYRMVQDLREVNKRVETIHPTVPNPYTLLSLLPPDRIWYSVLDLKDAFFCIPLAPESQLIFAFEWADAEEGESGQLTWTRLPQGFKNSPTLFN |
| 3mutA | | EALNRDLQGFRLDHPSVSLLQYVDDLLIAADTQAACLSATRDLLMTLAELGYRVSGKKAQLCQEEVTYLGFKIHKGSRSLSNSRTQAILQIPVPKTKRQV |
| | | REFLGKIGYCRLFIPGFAELAQPLYAATRPGNDPLVWGEKEEEAFQSLKLALTQPPALALPSLDKPFQLFVEETSGAAKGVLTQALGPWKRPVAYLSKR |
| | | LDPVAAGWPRCLRAIAAAALLTREASKLTFGQDIEITSSHNLESLLRSPPDKWLTNARITQYQVLLLDPPRVRFKQTAALNPATLLPETDDTLPIHHCLDT |
| | | LDSLTSTRPDLTDQPLAQAEATLFTDGSSYIRDGKRYAGAAVVTLDSVIWAEPLPIGTSAQKAELIALTKALEWSKDKSVNIYTDSRYAFATLHVHGMIY |
| | | RERGWLTAGGKAIKNAPEILALLTAVWLPKRVAVMHCKGHQKDDAPTSTGNRRADEVAREVAIRPLSTQATIS |
| FLV_ | 8,020 | TLQLEEEYRLFEPESTQKQEMDIWLKNFPQAWAETGGMGTAHCQAPVLIQLKATATPISIRQYPMPHEAYQGIKPHIRRMLDQGILKPCQSPWNTPLLP |
| P10273_ | | VKKPGTEDYRPVQDLREVNKRVEDIHPTVPNPYNLLSTLPPSHPWYTVLDLKDAFFCLRLHSESQLLFAFEWRDPEIGLSGQLTWTRLPQGFKNSPTL |
| 3mutA | | FNEALHSDLADFRVRYPALVLLQYVDDLLLAAATRTECLEGTKALLETLGNKGYRASAKKAQICLQEVTYLGYSLKDGQRWLTKARKEAILSIPVPKNSR |
| | | QVREFLGKAGYCRLFIPGFAELAAPLYPLTRPGTLFQWGTEQQLAFEDIKKALLSSPALGLPDITKPFELFIDENSGFAKGVLVQKLGPWKRPVAYLSKK |
| | | LDTVASGWPPCLRMVAAIAILVKDAGKLTLGQPLTILTSHPVEALVRQPPNKWLSNARMTHYQAMLLDAERVHFGPTVSLNPATLLPLPSGGNHHDCL |
| | | QILAETHGTRPDLTDQPLPDADLTWYTDGSSFIRNGEREAGAAVTTESEVIWAAPLPPGTSAQRAELIALTQALKMAEGKKLTVYTDSRYAFATTHVHG |
| | | EIYRRRGWLTSEGKEIKNKNEILALLEALFLPKRLSIIHCPGHQKGDSPQAKGNRLADDTAKKAATETHSSLTVLP |
| MLVMS_ | 8,074 | TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLP |
| P03355_ | | VKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPT |
| 3mutA_ | | LFNEALHRDLADFRIQHPDLILLQYVDDLLLAATSELDCQQGTRALLQTLGNLGYRASAKKAQICQKQVKYLGYLLKEGQRWLTEARKETVMGQPTPKT |
| WS | | PRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAY |
| | | LSKKLDPVAAGWPPCLRMVAAIAVLTKDAGKLTMGQPLVILAPHAVEALVKQPPDRWLSNARMTHYQALLLDTDRVQFGPWVALNPATLLPLPEEGLQ |
| | | HNCLDILAEAHGTRPDLTDQPLPDADHTWYTDGSSLLQEGQRKAGAAVTTETEVIWAKALPAGTSAQRAELIALTQALKMAEGKKLNVYTDSRYAFAT |
| | | AHIHGEIYRRRGWLTSEGKEIKNKDEILALLKALFLPKRLSIIHCPGHQKGHSAEARGNRMADQAARKAAITETPDTSTLL |
| SFV3L_ | 8,113 | MDPLQLLQPLEAEIKGTKLKAHWNSGATITCVPQAFLEEEVPIKNIWIKTIHGEKEQPVYYLTFKIQGRKVEAEVISSPYDYILVSPSDIPWLMKKPLQLTT |
| P27401_ | | LVPLQEYEERLLKQTMLTGSYKEKLQSLFLKYDALWQHWENQVGHRRIKPHHIATGTVNPRPQKQYPINPKAKASIQTVINDLLKQGVLIQQNSIMNTP |
| 2mutA | | VYPVPKPDGKWRMVLDYREVNKTIPLIAAQNQHSAGILSSIFRGKYKTTLDLSNGFWAHSITPESYWLTAFTWLGQQYCWTRLPQGFLNSPALFNADV |
| | | VDLLKEVPNVQVYVDDIYISHDDPREHLEQLEKVFSLLLNAGYVVSLKKSEIAQHEVEFLGFNITKEGRGLTETFKQKLLNITPPRDLKQLQSILGKLNFA |
| | | RNFIPNFSELVKPLYNIIATAPGKYITWTTDNSQQLQNIISMLNSAENLEERNPEVRLIMKVNTSPSAGYIRFYNEFAKRPIMYLNYVYTKAEVKFTNTEKL |
| | | LTTIHKGLIKALDLGMGQEILVYSPIVSMTKIQKTPLPERKALPIRWITWMSYLEDPRIQFHYDKTLPELQQVPTVTDDIIAKIKHPSEFSMVFYTDGSAIKH |
| | | PNVNKSHNAGMGIAQVQFKPEFTVINTWSIPLGDHTAQLAEVAAVEFACKKALKIDGPVLIVTDSFYVAESVNKELPYWQSNGFFNNKKKPLKHVSKW |
| | | KSIADCIQLKPDIIIIHEKGHQPTASTFHTEGNNLADKLATQGSYVVN |

In some embodiments, reverse transcriptase domains are modified, for example by site-specific mutation. In some embodiments, reverse transcriptase domains are engineered to have improved properties, e.g. SuperScript IV (SSIV) reverse transcriptase derived from the MMLV RT. In some embodiments, the reverse transcriptase domain may be engineered to have lower error rates, e.g., as described in WO2001068895, incorporated herein by reference. In some embodiments, the reverse transcriptase domain may be engineered to be more thermostable. In some embodiments, the reverse transcriptase domain may be engineered to be more processive. In some embodiments, the reverse transcriptase domain may be engineered to have tolerance to inhibitors. In some embodiments, the reverse transcriptase domain may be engineered to be faster. In some embodiments, the reverse transcriptase domain may be engineered to better tolerate modified nucleotides in the RNA template. In some embodiments, the reverse transcriptase domain may be engineered to insert modified DNA nucleotides. In some embodiments, the reverse transcriptase domain is engineered to bind a template RNA. In some embodiments, one or more mutations are chosen from D200N, L603W, T330P, D524G, E562Q, D583N, P51L, S67R, E67K, T197A, H204R, E302K, F309N, W313F, L435G, N454K, H594Q, L671P, E69K, H8Y, T306K, or D653N in the RT domain of murine leukemia virus reverse transcriptase or a corresponding mutation at a corresponding position of another RT domain.

In some embodiments, a heterologous gene modifying polypeptide comprises the RT domain from a retroviral reverse transcriptase, e.g., a wild-type M-MLV RT, e.g., comprising the following sequence:

M-MLV (WT):
(SEQ ID NO: 5002)
TLNIEDEYRLHETSKEPDVSLGSTWLSDFPQAWAETGGMGLAVRQAPLI
IPLKATSTPVSIKQYPMSQEARLGIKPHIQRLLDQGILVPCQSPWNTPL
LPVKKPGTNDYRPVQDLREVNKRVEDIHPTVPNPYNLLSGLPPSHQWYT
VLDLKDAFFCLRLHPTSQPLFAFEWRDPEMGISGQLTWTRLPQGFKNSP
TLFDEALHRDLADFRIQHPDLILLQYVDDLLLAATSELDCQQGTRALLQ
TLGNLGYRASAKKAQICQKQVKYLGYLLKEGQRWLTEARKETVMGQPTP
KTPRQLREFLGTAGFCRLWIPGFAEMAAPLYPLTKTGTLFNWGPDQQKA
YQEIKQALLTAPALGLPDLTKPFELFVDEKQGYAKGVLTQKLGPWRRPV
AYLSKKLDPVAAGWPPCLRMVAAIAVLTKDAGKLTMGQPLVILAPHAVE
ALVKQPPDRWLSNARMTHYQALLLDTDRVQFGPVVALNPATLLPLPEEG
LQHNCLDILAEAHGTRPDLTDQPLPDADHTWYTDGSSLLQEGQRKAGAA
VTTETEVIWAKALPAGTSAQRAELIALTQALKMAEGKKLNVYTDSRYAF
ATAHIHGEIYRRRGLLTSEGKEIKNKDEILALLKALFLPKRLSIIHCPG
HQKGHSAEARGNRMADQAARKAAITETPDTSTLLI

In some embodiments, a heterologous gene modifying polypeptide comprises the RT domain from a retroviral reverse transcriptase, e.g., an M-MLV RT, e.g., comprising the following sequence:

(SEQ ID NO: 5003)
TLNIEDEHRLHETSKEPDVSLGSTWLSDFPQAWAETGGMGLAVRQAPLI
IPLKATSTPVSIKQYPMSQEARLGIKPHIQRLLDQGILVPCQSPWNTPL
LPVKKPGTNDYRPVQDLREVNKRVEDIHPTVPNPYNLLSGLPPSHQWYT
VLDLKDAFFCLRLHPTSQPLFAFEWRDPEMGISGQLTWTRLPQGFKNSP
TLFDEALHRDLADFRIQHPDLILLQYVDDLLLAATSELDCQQGTRALLQ
TLGNLGYRASAKKAQICQKQVKYLGYLLKEGQRWLTEARKETVMGQPTP
KTPRQLREFLGTAGFCRLWIPGFAEMAAPLYPLTKTGTLFNWGPDQQKA
YQEIKQALLTAPALGLPDLTKPFELFVDEKQGYAKGVLTQKLGPWRRPV
AYLSKKLDPVAAGWPPCLRMVAAIAVLTKDAGKLTMGQPLVILAPHAVE
ALVKQPPDRWLSNARMTHYQALLLDTDRVQFGPVVALNPATLLPLPEEG
LQHNCLDILAEAHGTRPDLTDQPLPDADHTWYTDGSSLLQEGQRKAGAA
VTTETEVIWAKALPAGTSAQRAELIALTQALKMAEGKKLNVYTDSRYAF
ATAHIHGEIYRRRGLLTSEGKEIKNKDEILALLKALFLPKRLSIIHCPG
HQKGHSAEARGNRMADQAARKAAITETPDTSTLL

In some embodiments, a heterologous gene modifying polypeptide comprises the RT domain from a retroviral reverse transcriptase comprising the sequence of amino acids 659-1329 of NP_057933. In embodiments, the heterologous gene modifying polypeptide further comprises one additional amino acid at the N-terminus of the sequence of amino acids 659-1329 of NP_057933, e.g., as shown below:

(SEQ ID NO: 5004)
TLNIEDEHRLHETSKEPDVSLGSTWLSDFPQAWAETGGMGLAVRQAPLI
IPLKATSTPVSIKQYPMSQEARLGIKPHIQRLLDQGILVPCQSPWNTPL
LPVKKPGTNDYRPVQDLREVNKRVEDIHPTVPNPYNLLSGLPPSHQWYT
VLDLKDAFFCLRLHPTSQPLFAFEWRDPEMGISGQLTWTRLPQGFKNSP
TLFDEALHRDLADFRIQHPDLILLQYVDDLLLAATSELDCQQGTRALLQ
TLGNLGYRASAKKAQICQKQVKYLGYLLKEGQRWLTEARKETVMGQPTP
KTPRQLREFLGTAGFCRLWIPGFAEMAAPLYPLTKTGTLFNWGPDQQKA
YQEIKQALLTAPALGLPDLTKPFELFVDEKQGYAKGVLTQKLGPWRRPV
AYLSKKLDPVAAGWPPCLRMVAAIAVLTKDAGKLTMGQPLVILAPHAVE
ALVKQPPDRWLSNARMTHYQALLLDTDRVQFGPVVALNPATLLPLPEEG
LQHNCLDILAEAHGTRPDLTDQPLPDADHTWYTDGSSLLQEGQRKAGAA
VTTETEVIWAKALPAGTSAQRAELIALTQALKMAEGKKLNVYTDSRYAF
ATAHIHGEIYRRRGLLTSEGKEIKNKDEILALLKALFLPKRLSIIHCPG
HQKGHSAEARGNRMADQAARKAA

Core RT (bold), annotated per above
RNAseH (underlined), annotated per above

In embodiments, the heterologous gene modifying polypeptide further comprises one additional amino acid at the C-terminus of the sequence of amino acids 659-1329 of NP_057933. In embodiments, the heterologous gene modifying polypeptide comprises an RNaseH1 domain (e.g., amino acids 1178-1318 of NP_057933).

In some embodiments, a retroviral reverse transcriptase domain, e.g., M-MLV RT, may comprise one or more mutations from a wild-type sequence that may improve features of the RT, e.g., thermostability, processivity, and/or template binding. In some embodiments, an M-MLV RT domain comprises, relative to the M-MLV (WT) sequence above, one or more mutations, e.g., selected from D200N, L603W, T330P, T306K, W313F, D524G, E562Q, D583N, P51L, S67R, E67K, T197A, H204R, E302K, F309N, L435G, N454K, H594Q, D653N, R110S, K103L, e.g., a combination of mutations, such as D200N, L603W, and T330P, optionally further including T306K and W313F. In some embodiments, an M-MLV RT used herein comprises the mutations D200N, L603W, T330P, T306K, and W313F. In embodiments, the mutant M-MLV RT comprises the following amino acid sequence:

M-MLV (PE2):
(SEQ ID NO: 5005)
TLNIEDEYRLHETSKEPDVSLGSTWLSDFPQAWAETGGMGLAVRQAPLI
IPLKATSTPVSIKQYPMSQEARLGIKPHIQRLLDQGILVPCQSPWNTPL
LPVKKPGTNDYRPVQDLREVNKRVEDIHPTVPNPYNLLSGLPPSHQWYT
VLDLKDAFFCLRLHPTSQPLFAFEWRDPEMGISGQLTWTRLPQGFKNSP
TLFNEALHRDLADFRIQHPDLILLQYVDDLLLAATSELDCQQGTRALLQ
TLGNLGYRASAKKAQICQKQVKYLGYLLKEGQRWLTEARKETVMGQPTP
KTPRQLREFLGKAGFCRLFIPGFAEMAAPLYPLTKPGTLFNWGPDQQKA
YQEIKQALLTAPALGLPDLTKPFELFVDEKQGYAKGVLTQKLGPWRRPV
AYLSKKLDPVAAGWPPCLRMVAAIAVLTKDAGKLTMGQPLVILAPHAVE
ALVKQPPDRWLSNARMTHYQALLLDTDRVQFGPVVALNPATLLPLPEEG
LQHNCLDILAEAHGTRPDLTDQPLPDADHTWYTDGSSLLQEGQRKAGAA
VTTETEVIWAKALPAGTSAQRAELIALTQALKMAEGKKLNVYTDSRYAF
ATAHIHGEIYRRRGWLTSEGKEIKNKDEILALLKALFLPKRLSIIHCPG
HQKGHSAEARGNRMADQAARKAAITETPDTSTLLI

In some embodiments, a writing domain (e.g., RT domain) comprises an RNA-binding domain, e.g., that specifically binds to an RNA sequence. In some embodiments, a template RNA comprises an RNA sequence that is specifically bound by the RNA-binding domain of the writing domain.

In some embodiments, the reverse transcription domain only recognizes and reverse transcribes a specific template, e.g., a template RNA of the system. In some embodiments, the template comprises a sequence or structure that enables recognition and reverse transcription by a reverse transcription domain. In some embodiments, the template comprises a sequence or structure that enables association with an RNA-binding domain of a polypeptide component of a genome engineering system described herein. In some embodiments, the genome engineering system reverse preferably transcribes a template comprising an association sequence over a template lacking an association sequence.

The writing domain may also comprise DNA-dependent DNA polymerase activity, e.g., comprise enzymatic activity capable of writing DNA into the genome from a template DNA sequence. In some embodiments, DNA-dependent DNA polymerization is employed to complete second-strand synthesis of a target site edit. In some embodiments, the DNA-dependent DNA polymerase activity is provided by a DNA polymerase domain in the polypeptide. In some embodiments, the DNA-dependent DNA polymerase activity is provided by a reverse transcriptase domain that is also capable of DNA-dependent DNA polymerization, e.g., second-strand synthesis. In some embodiments, the DNA-dependent DNA polymerase activity is provided by a second polypeptide of the system. In some embodiments, the DNA-dependent DNA polymerase activity is provided by an endogenous host cell polymerase that is optionally recruited to the target site by a component of the genome engineering system.

In some embodiments, the reverse transcriptase domain has a lower probability of premature termination rate (P_{off}) in vitro relative to a reference reverse transcriptase domain. In some embodiments, the reference reverse transcriptase domain is a viral reverse transcriptase domain, e.g., the RT domain from M-MLV.

In some embodiments, the reverse transcriptase domain has a lower probability of premature termination rate (P_{off}) in vitro of less than about 5×10⁻³/nt, 5×10⁻⁴/nt, or 5×10⁻⁶/nt, e.g., as measured on a 1094 nt RNA. In embodiments, the in vitro premature termination rate is determined as described in Bibillo and Eickbush (2002) J Biol Chem 277(38):34836-34845 (incorporated by reference herein its entirety).

In some embodiments, the reverse transcriptase domain is able to complete at least about 30% or 50% of integrations in cells. The percent of complete integrations can be measured by dividing the number of substantially full-length integration events (e.g., genomic sites that comprise at least 98% of the expected integrated sequence) by the number of total (including substantially full-length and partial) integration events in a population of cells. In embodiments, the integrations in cells is determined (e.g., across the integration site) using long-read amplicon sequencing, e.g., as described in Karst et al. (2020) bioRxiv doi.org/10.1101/645903 (incorporated by reference herein in its entirety).

In embodiments, quantifying integrations in cells comprises counting the fraction of integrations that contain at least about 75%, 80%, 85%, 90%, 95%, 96%, 97%, 98%, 99%, or 100% of the DNA sequence corresponding to the template RNA (e.g., a template RNA having a length of at least 0.05, 0.1, 0.5, 0.6, 0.7, 0.8, 0.9, 1, 1.5, 2, 3, 4, or 5 kb, e.g., a length between 0.5-0.6, 0.6-0.7, 0.7-0.8, 0.8-0.9, 1.0-1.2, 1.2-1.4, 1.4-1.6, 1.6-1.8, 1.8-2.0, 2-3, 3-4, or 4-5 kb).

In some embodiments, the reverse transcriptase domain is capable of polymerizing dNTPs in vitro. In embodiments, the reverse transcriptase domain is capable of polymerizing dNTPs in vitro at a rate between 0.1-50 nt/sec (e.g., between 0.1-1, 1-10, or 10-50 nt/sec). In embodiments, polymerization of dNTPs by the reverse transcriptase domain is measured by a single-molecule assay, e.g., as described in Schwartz and Quake (2009) PNAS 106(48):20294-20299 (incorporated by reference in its entirety).

In some embodiments, the reverse transcriptase domain has an in vitro error rate (e.g., misincorporation of nucleotides) of between 1×10⁻³-1×10⁻⁴ or 1×10⁻⁴-1×10⁻⁵ substitutions/nt, e.g., as described in Yasukawa et al. (2017) Biochem Biophys Res Commun 492(2):147-153 (incorporated herein by reference in its entirety). In some embodiments, the reverse transcriptase domain has an error rate (e.g., misincorporation of nucleotides) in cells (e.g., HEK293T cells) of between 1×10⁻³-1×10⁻⁴ or 1×10⁻⁴-1×10⁻⁵ substitutions/nt, e.g., by long-read amplicon sequencing, e.g., as described in Karst et al. (2020) bioRxiv doi.org/10.1101/645903 (incorporated by reference herein in its entirety).

In some embodiments, the reverse transcriptase domain is capable of performing reverse transcription of a target RNA in vitro. In some embodiments, the reverse transcriptase requires a primer of at least 3 nucleotides to initiate reverse transcription of a template. In some embodiments, reverse transcription of the target RNA is determined by detection of cDNA from the target RNA (e.g., when provided with a ssDNA primer, e.g., which anneals to the target with at least 3, 4, 5, 6, 7, 8, 9, or 10 nt at the 3′ end), e.g., as described in Bibillo and Eickbush (2002) J Biol Chem 277(38):34836-34845 (incorporated herein by reference in its entirety).

In some embodiments, the reverse transcriptase domain performs reverse transcription at least 5 or 10 times more efficiently (e.g., by cDNA production), e.g., when converting its RNA template to cDNA, for example, as compared to an RNA template lacking the protein binding motif (e.g., a 3′ UTR). In embodiments, efficiency of reverse transcription is measured as described in Yasukawa et al. (2017) Biochem Biophys Res Commun 492(2):147-153 (incorporated by reference herein in its entirety).

In some embodiments, the reverse transcriptase domain specifically binds a specific RNA template with higher frequency (e.g., about 5 or 10-fold higher frequency) than any endogenous cellular RNA, e.g., when expressed in cells (e.g., HEK293T cells). In embodiments, frequency of specific binding between the reverse transcriptase domain and the template RNA are measured by CLIP-seq, e.g., as described in Lin and Miles (2019) Nucleic Acids Res 47(11):5490-5501 (incorporated herein by reference in its entirety).

### Template Nucleic Acid Binding Domain

The heterologous gene modifying polypeptide typically contains regions capable of associating with the template nucleic acid (e.g., template RNA). In some embodiments, the template nucleic acid binding domain is an RNA binding domain. In some embodiments, the RNA binding domain is a modular domain that can associate with RNA molecules containing specific signatures, e.g., structural motifs. In other embodiments, the template nucleic acid binding domain (e.g., RNA binding domain) is contained within the reverse transcription domain, e.g., the reverse transcriptase-derived component has a known signature for RNA preference.

In other embodiments, the template nucleic acid binding domain (e.g., RNA binding domain) is contained within the target DNA binding domain. For example, in some embodiments, the DNA binding domain is a CRISPR-associated protein that recognizes the structure of a template nucleic acid (e.g., template RNA) comprising a gRNA. In some embodiments, a heterologous gene modifying polypeptide comprises a DNA-binding domain comprising a CRISPR-associated protein that associates with a gRNA scaffold that allows the DNA-binding domain to bind a target genomic DNA sequence. In some embodiments, the gRNA scaffold and gRNA spacer is comprised within the template nucleic acid (e.g., template RNA), thus the DNA-binding domain is also the template nucleic acid binding domain. In some embodiments, the polypeptide possesses RNA binding function in multiple domains, e.g., can bind a gRNA structure in a CRISPR-associated DNA binding domain and an additional sequence or structure in a reverse transcriptase domain.

In some embodiments, the RNA binding domain is capable of binding to a template RNA with greater affinity than a reference RNA binding domain. In some embodiments, the reference RNA binding domain is an RNA binding domain from Cas9 of S. pyogenes. In some embodiments, the RNA binding domain is capable of binding to a template RNA with an affinity between 100 pM-10 nM (e.g., between 100 pM-1 nM or 1 nM-10 nM). In some embodiments, the affinity of a RNA binding domain for its template RNA is measured in vitro, e.g., by thermophoresis, e.g., as described in Asmari et al. Methods 146:107-119 (2018) (incorporated by reference herein in its entirety). In some embodiments, the affinity of a RNA binding domain for its template RNA is measured in cells (e.g., by FRET or CLIP-Seq).

In some embodiments, the RNA binding domain is associated with the template RNA in vitro at a frequency at least about 5-fold or 10-fold higher than with a scrambled RNA. In some embodiments, the frequency of association between the RNA binding domain and the template RNA or scrambled RNA is measured by CLIP-seq, e.g., as described in Lin and Miles (2019) Nucleic Acids Res 47(11):5490-5501 (incorporated by reference herein in its entirety). In some embodiments, the RNA binding domain is associated with the template RNA in cells (e.g., in HEK293T cells) at a frequency at least about 5-fold or 10-fold higher than with a scrambled RNA. In some embodiments, the frequency of association between the RNA binding domain and the template RNA or scrambled RNA is measured by CLIP-seq, e.g., as described in Lin and Miles (2019), supra.

### Endonuclease Domains and DNA Binding Domains

In some embodiments, a heterologous gene modifying polypeptide possesses the function of DNA target site cleavage via an endonuclease domain. In some embodiments, a heterologous gene modifying polypeptide comprises a DNA binding domain, e.g., for binding to a target nucleic acid. In some embodiments, a domain (e.g., a Cas domain) of the heterologous gene modifying polypeptide comprises two or more smaller domains, e.g., a DNA binding domain and an endonuclease domain. It is understood that when a DNA binding domain (e.g., a Cas domain) is said to bind to a target nucleic acid sequence, in some embodiments, the binding is mediated by a gRNA.

In some embodiments, a domain has two functions. For example, in some embodiments, the endonuclease domain is also a DNA-binding domain. In some embodiments, the endonuclease domain is also a template nucleic acid (e.g., template RNA) binding domain. For example, in some embodiments, a polypeptide comprises a CRISPR-associated endonuclease domain that binds a template RNA comprising a gRNA, binds a target DNA sequence (e.g., with complementarity to a portion of the gRNA), and cuts the target DNA sequence. In some embodiments, an endonuclease domain or endonuclease/DNA-binding domain from a heterologous source can be used or can be modified (e.g., by insertion, deletion, or substitution of one or more residues) in a system described herein.

In some embodiments, a nucleic acid encoding the endonuclease domain or endonuclease/DNA binding domain is altered from its natural sequence to have altered codon usage, e.g. improved for human cells. In some embodiments, the endonuclease element is a heterologous endonuclease element, such as a Cas endonuclease (e.g., Cas9), a type-II restriction endonuclease (e.g., Fok1), a meganuclease (e.g., I-SceI), or other endonuclease domain.

In certain aspects, the DNA-binding domain of a heterologous gene modifying polypeptide described herein is selected, designed, or constructed for binding to a desired host DNA target sequence. In certain embodiments, the DNA-binding domain of the polypeptide is a heterologous DNA-binding element. In some embodiments the heterologous DNA binding element is a zinc-finger element or a TAL effector element, e.g., a zinc-finger or TAL polypeptide or functional fragment thereof. In some embodiments the heterologous DNA binding element is a sequence-guided DNA binding element, such as Cas9, Cpf1, or other CRISPR-related protein that has been altered to have no endonuclease activity. In some embodiments the heterologous DNA binding element retains endonuclease activity. In some embodiments, the heterologous DNA binding element retains partial endonuclease activity to cleave ssDNA, e.g., possesses nickase activity. In specific embodiments, the heterologous DNA-binding domain can be any one or more of Cas9, TAL domain, ZF domain, Myb domain, combinations thereof, or multiples thereof.

In some embodiments, DNA-binding domains are modified, for example by site-specific mutation, increasing or decreasing DNA-binding elements (for example, number and/or specificity of zinc fingers), etc., to alter DNA-binding specificity and affinity. In some embodiments a nucleic acid sequence encoding the DNA binding domain is altered from its natural sequence to have altered codon usage, e.g. improved for human cells. In embodiments, the DNA binding domain comprises one or more modifications relative to a wild-type DNA binding domain, e.g., a modification via directed evolution, e.g., phage-assisted continuous evolution (PACE).

In some embodiments, the DNA binding domain comprises a meganuclease domain (e.g., as described herein, e.g., in the endonuclease domain section), or a functional fragment thereof. In some embodiments, the meganuclease domain possesses endonuclease activity, e.g., double-strand cleavage and/or nickase activity. In other embodiments, the meganuclease domain has reduced activity, e.g., lacks endonuclease activity, e.g., the meganuclease is catalytically inactive. In some embodiments, a catalytically inactive meganuclease is used as a DNA binding domain, e.g., as described in Fonfara et al. Nucleic Acids Res 40(2):847-860 (2012), incorporated herein by reference in its entirety.

In some embodiments, a heterologous gene modifying polypeptide comprises a modification to a DNA-binding domain, e.g., relative to the wild-type polypeptide. In some embodiments, the DNA-binding domain comprises an addition, deletion, replacement, or modification to the amino acid sequence of the original DNA-binding domain. In some embodiments, the DNA-binding domain is modified to include a heterologous functional domain that binds specifically to a target nucleic acid (e.g., DNA) sequence of interest. In some embodiments, the functional domain replaces at least a portion (e.g., the entirety of) the prior DNA-binding domain of the polypeptide. In some embodiments, the functional domain comprises a zinc finger (e.g., a zinc finger that specifically binds to the target nucleic acid (e.g., DNA) sequence of interest. In some embodiments, the functional domain comprises a Cas domain (e.g., a Cas domain that specifically binds to the target nucleic acid (e.g., DNA) sequence of interest. In some embodiments, the Cas domain comprises a Cas9 or a mutant or variant thereof (e.g., as described herein). In embodiments, the Cas domain is associated with a guide RNA (gRNA), e.g., as described herein. In embodiments, the Cas domain is directed to a target nucleic acid (e.g., DNA) sequence of interest by the gRNA. In embodiments, the Cas domain is encoded in the same nucleic acid (e.g., RNA) molecule as the gRNA. In embodiments, the Cas domain is encoded in a different nucleic acid (e.g., RNA) molecule from the gRNA.

In some embodiments, the DNA binding domain is capable of binding to a target sequence (e.g., a dsDNA target sequence) with greater affinity than a reference DNA binding domain. In some embodiments, the reference DNA binding domain is a DNA binding domain from Cas9 of S. pyogenes. In some embodiments, the DNA binding domain is capable of binding to a target sequence (e.g., a dsDNA target sequence) with an affinity between 100 pM-10 nM (e.g., between 100 pM-1 nM or 1 nM-10 nM).

In some embodiments, the affinity of a DNA binding domain for its target sequence (e.g., dsDNA target sequence) is measured in vitro, e.g., by thermophoresis, e.g., as described in Asmari et al. Methods 146:107-119 (2018) (incorporated by reference herein in its entirety).

In embodiments, the DNA binding domain is capable of binding to its target sequence (e.g., dsDNA target sequence), e.g, with an affinity between 100 pM-10 nM (e.g., between 100 pM-1 nM or 1 nM-10 nM) in the presence of a molar excess of scrambled sequence competitor dsDNA, e.g., of about 100-fold molar excess.

In some embodiments, the DNA binding domain is found associated with its target sequence (e.g., dsDNA target sequence) more frequently than any other sequence in the genome of a target cell, e.g., human target cell, e.g., as measured by ChIP-seq (e.g., in HEK293T cells), e.g., as described in He and Pu (2010) Curr. Protoc Mol Biol Chapter 21 (incorporated herein by reference in its entirety). In some embodiments, the DNA binding domain is found associated with its target sequence (e.g., dsDNA target sequence) at least about 5-fold or 10-fold, more frequently than any other sequence in the genome of a target cell, e.g., as measured by ChIP-seq (e.g., in HEK293T cells), e.g., as described in He and Pu (2010), supra.

In some embodiments, the endonuclease domain has nickase activity and cleaves one strand of a target DNA. In some embodiments, nickase activity reduces the formation of double-stranded breaks at the target site. In some embodiments, the endonuclease domain creates a staggered nick structure in the first and second strands of a target DNA. In some embodiments, a staggered nick structure generates free 3′ overhangs at the target site. In some embodiments, free 3′ overhangs at the target site improve editing efficiency, e.g., by enhancing access and annealing of a 3′ homology region of a template nucleic acid. In some embodiments, a staggered nick structure reduces the formation of double-stranded breaks at the target site.

In some embodiments, the endonuclease domain cleaves both strands of a target DNA, e.g., results in blunt-end cleavage of a target with no ssDNA overhangs on either side of the cut-site. The amino acid sequence of an endonuclease domain of a system described herein may be at least about 50%, at least about 60%, at least about 70%, at least about 80%, at least about 85%, at least about 90%, at least about 95%, at least about 96%, at least about 97%, at least about 98%, at least about 99% identical to the amino acid sequence of an endonuclease domain described herein, e.g., an endonuclease domain from Table 8A.

In certain embodiments, the heterologous endonuclease is Fok1 or a functional fragment thereof. In certain embodiments, the heterologous endonuclease is a Holliday junction resolvase or homolog thereof, such as the Holliday junction resolving enzyme from Sulfolobus solfataricus—Ssol Hje (Govindaraju et al., Nucleic Acids Research 44:7, 2016). In certain embodiments, the heterologous endonuclease is the endonuclease of the large fragment of a spliceosomal protein, such as Prp8 (Mahbub et al., Mobile DNA 8:16, 2017). In certain embodiments, the heterologous endonuclease is derived from a CRISPR-associated protein, e.g., Cas9. In certain embodiments, the heterologous endonuclease is engineered to have only ssDNA cleavage activity, e.g., only nickase activity, e.g., be a Cas9 nickase, e.g., SpCas9 with D10A, H840A, or N863A mutations. Table 8A provides exemplary Cas proteins and mutations associated with nickase activity. In still other embodiments, homologous endonuclease domains are modified, for example by site-specific mutation, to alter DNA endonuclease activity. In still other embodiments, endonuclease domains are modified to reduce DNA-sequence specificity, e.g., by truncation to remove domains that confer DNA-sequence specificity or mutation to inactivate regions conferring DNA-sequence specificity.

In some embodiments, the endonuclease domain has nickase activity and does not form double-stranded breaks. In some embodiments, the endonuclease domain forms single-stranded breaks at a higher frequency than double-stranded breaks, e.g., at least 90%, 95%, 96%, 97%, 98%, or 99% of the breaks are single-stranded breaks, or less than 10%, 5%, 4%, 3%, 2%, or 1% of the breaks are double-stranded breaks. In some embodiments, the endonuclease forms substantially no double-stranded breaks. In some embodiments, the endonuclease does not form detectable levels of double-stranded breaks.

In some embodiments, the endonuclease domain has nickase activity that nicks the target site DNA of the first strand; e.g., in some embodiments, the endonuclease domain cuts the genomic DNA of the target site near to the site of alteration on the strand that will be extended by the writing domain. In some embodiments, the endonuclease domain has nickase activity that nicks the target site DNA of the first strand and does not nick the target site DNA of the second strand. For example, when a polypeptide comprises a CRISPR-associated endonuclease domain having nickase activity, in some embodiments, said CRISPR-associated endonuclease domain nicks the target site DNA strand containing the PAM site (e.g., and does not nick the target site DNA strand that does not contain the PAM site). As a further example, when a polypeptide comprises a CRISPR-associated endonuclease domain having nickase activity, in some embodiments, said CRISPR-associated endonuclease domain nicks the target site DNA strand not containing the PAM site (e.g., and does not nick the target site DNA strand that contains the PAM site).

In some other embodiments, the endonuclease domain has nickase activity that nicks the target site DNA of the first strand and the second strand. Without wishing to be bound by theory, after a writing domain (e.g., RT domain) of a polypeptide described herein polymerizes (e.g., reverse transcribes) from the heterologous object sequence of a template nucleic acid (e.g., template RNA), the cellular DNA repair machinery must repair the nick on the first DNA strand. The target site DNA now contains two different sequences for the first DNA strand: one corresponding to the original genomic DNA (e.g., having a free 5′ end) and a second corresponding to that polymerized from the heterologous object sequence (e.g., having a free 3′ end). It is thought that the two different sequences equilibrate with one another, first one hybridizing the second strand, then the other, and which sequence the cellular DNA repair apparatus incorporates into its repaired target site may be a stochastic process. Without wishing to be bound by theory, it is thought that introducing an additional nick to the second-strand may bias the cellular DNA repair machinery to adopt the heterologous object sequence-based sequence more frequently than the original genomic sequence (Anzalone et al. Nature 576:149-157 (2019)). In some embodiments, the additional nick is positioned at least 10, 15, 20, 25, 30, 35, 40, 45, 50, 55, 60, 65, 70, 75, 80, 85, 90, 95, 100, 105, 110, 115, 120, 125, 130, 135, 140, 145, or 150 nucleotides 5′ or 3′ of the target site modification (e.g., the insertion, deletion, or substitution) or to the nick on the first strand.

Alternatively or additionally, without wishing to be bound by theory, it is thought that an additional nick to the second strand may promote second-strand synthesis. In some embodiments, where the system has inserted or substituted a portion of the first strand, synthesis of a new sequence corresponding to the insertion/substitution in the second strand is necessary.

In some embodiments, the polypeptide comprises a single domain having endonuclease activity (e.g., a single endonuclease domain) and said domain nicks both the first strand and the second strand. For example, in such an embodiment the endonuclease domain may be a CRISPR-associated endonuclease domain, and the template nucleic acid (e.g., template RNA) comprises a gRNA spacer that directs nicking of the first strand and an additional gRNA spacer that directs nicking of the second strand. In some embodiments, the polypeptide comprises a plurality of domains having endonuclease activity, and a first endonuclease domain nicks the first strand and a second endonuclease domain nicks the second strand (optionally, the first endonuclease domain does not (e.g., cannot) nick the second strand and the second endonuclease domain does not (e.g., cannot) nick the first strand).

In some embodiments, the endonuclease domain is capable of nicking a first strand and a second strand. In some embodiments, the first and second strand nicks occur at the same position in the target site but on opposite strands. In some embodiments, the second strand nick occurs in a staggered location, e.g., upstream or downstream, from the first nick. In some embodiments, the endonuclease domain generates a target site deletion if the second strand nick is upstream of the first strand nick. In some embodiments, the endonuclease domain generates a target site duplication if the second strand nick is downstream of the first strand nick. In some embodiments, the endonuclease domain generates no duplication and/or deletion if the first and second strand nicks occur in the same position of the target site. In some embodiments, the endonuclease domain has altered activity depending on protein conformation or RNA-binding status, e.g., which promotes the nicking of the first or second strand (e.g., as described in Christensen et al. PNAS 2006; incorporated by reference herein in its entirety).

In some embodiments, the endonuclease domain comprises a meganuclease, or a functional fragment thereof. In some embodiments, the endonuclease domain comprises a homing endonuclease, or a functional fragment thereof. In some embodiments, the endonuclease domain comprises a meganuclease from the LAGLIDADG (SEQ ID NO: 15432), GIY-YIG, HNH, His-Cys Box, or PD-(D/E) XK families, or a functional fragment or variant thereof, e.g., which possess conserved amino acid motifs, e.g., as indicated in the family names. In some embodiments, the endonuclease domain comprises a meganuclease, or fragment thereof, chosen from, e.g., I-SmaMI (Uniprot F7WD42), I-SceI (Uniprot P03882), I-Anil (Uniprot P03880), I-DmoI (Uniprot P21505), I-CreI (Uniprot P05725), I-TevI (Uniprot P13299), I-OnuI (Uniprot Q4VWW5), or I-BmoI (Uniprot Q9ANR6). In some embodiments, the meganuclease is naturally monomeric, e.g., I-SceI, I-TevI, or dimeric, e.g., I-CreI, in its functional form. For example, the LAGLIDADG meganucleases (SEQ ID NO: 15432) with a single copy of the LAGLIDADG motif (SEQ ID NO: 15432) generally form homodimers, whereas members with two copies of the LAGLIDADG motif (SEQ ID NO: 15432) are generally found as monomers. In some embodiments, a meganuclease that normally forms as a dimer is expressed as a fusion, e.g., the two subunits are expressed as a single ORF and, optionally, connected by a linker, e.g., an I-CreI dimer fusion (Rodriguez-Fornes et al. Gene Therapy 2020; incorporated by reference herein in its entirety). In some embodiments, a meganuclease, or a functional fragment thereof, is altered to favor nickase activity for one strand of a double-stranded DNA molecule, e.g., I-SceI (K122I and/or K223I) (Niu et al. J Mol Biol 2008), I-Anil (K227M) (McConnell Smith et al. PNAS 2009), I-DmoI (Q42A and/or K120M) (Molina et al. J Biol Chem 2015). In some embodiments, a meganuclease or functional fragment thereof possessing this preference for single-strand cleavage is used as an endonuclease domain, e.g., with nickase activity. In some embodiments, an endonuclease domain comprises a meganuclease, or a functional fragment thereof, which naturally targets or is engineered to target a safe harbor site, e.g., an I-CreI targeting SH6 site (Rodriguez-Fornes et al., supra). In some embodiments, an endonuclease domain comprises a meganuclease, or a functional fragment thereof, with a sequence tolerant catalytic domain, e.g., I-TevI recognizing the minimal motif CNNNG (Kleinstiver et al. PNAS 2012). In some embodiments, a target sequence tolerant catalytic domain is fused to a DNA binding domain, e.g., to direct activity, e.g., by fusing I-TevI to: (i) zinc fingers to create Tev-ZFEs (Kleinstiver et al. PNAS 2012), (ii) other meganucleases to create MegaTevs (Wolfs et al. Nucleic Acids Res 2014), and/or (iii) Cas9 to create TevCas9 (Wolfs et al. PNAS 2016).

In some embodiments, the endonuclease domain comprises a restriction enzyme, e.g., a Type IIS or Type IIP restriction enzyme. In some embodiments, the endonuclease domain comprises a Type IIS restriction enzyme, e.g., FokI, or a fragment or variant thereof. In some embodiments, the endonuclease domain comprises a Type IIP restriction enzyme, e.g., PvuII, or a fragment or variant thereof. In some embodiments, a dimeric restriction enzyme is expressed as a fusion such that it functions as a single chain, e.g., a FokI dimer fusion (Minczuk et al. Nucleic Acids Res 36(12):3926-3938 (2008)).

The use of additional endonuclease domains is described, for example, in Guha and Edgell Int J Mol Sci 18(22):2565 (2017), which is incorporated herein by reference in its entirety.

In some embodiments, a heterologous gene modifying polypeptide comprises a modification to an endonuclease domain, e.g., relative to a wild-type Cas protein. In some embodiments, the endonuclease domain comprises an addition, deletion, replacement, or modification to the amino acid sequence of the wild-type Cas protein. In some embodiments, the endonuclease domain is modified to include a heterologous functional domain that binds specifically to and/or induces endonuclease cleavage of a target nucleic acid (e.g., DNA) sequence of interest. In some embodiments, the endonuclease domain comprises a zinc finger. In embodiments, the endonuclease domain comprising the Cas domain is associated with a guide RNA (gRNA), e.g., as described herein. In some embodiments, the endonuclease domain is modified to include a functional domain that does not target a specific target nucleic acid (e.g., DNA) sequence. In embodiments, the endonuclease domain comprises a Fok1 domain.

In some embodiments, the endonuclease domain is associated with the target dsDNA in vitro at a frequency at least about 5-fold or 10-fold higher than with a scrambled dsDNA. In some embodiments, the endonuclease domain is associated with the target dsDNA in vitro at a frequency at least about 5-fold or 10-fold higher than with a scrambled dsDNA, e.g., in a cell (e.g., a HEK293T cell). In some embodiments, the frequency of association between the endonuclease domain and the target DNA or scrambled DNA is measured by ChIP-seq, e.g., as described in He and Pu (2010) Curr. Protoc Mol Biol Chapter 21 (incorporated by reference herein in its entirety).

In some embodiments, the endonuclease domain can catalyze the formation of a nick at a target sequence, e.g., to an increase of at least about 5-fold or 10-fold relative to a non-target sequence (e.g., relative to any other genomic sequence in the genome of the target cell). In some embodiments, the level of nick formation is determined using NickSeq, e.g., as described in Elacqua et al. (2019) bioRxiv doi.org/10.1101/867937 (incorporated herein by reference in its entirety).

In some embodiments, the endonuclease domain is capable of nicking DNA in vitro. In embodiments, the nick results in an exposed base. In embodiments, the exposed base can be detected using a nuclease sensitivity assay, e.g., as described in Chaudhry and Weinfeld (1995) Nucleic Acids Res 23(19):3805-3809 (incorporated by reference herein in its entirety). In embodiments, the level of exposed bases (e.g., detected by the nuclease sensitivity assay) is increased by at least 10%, 50%, or more relative to a reference endonuclease domain. In some embodiments, the reference endonuclease domain is an endonuclease domain from Cas9 of S. pyogenes.

In some embodiments, the endonuclease domain is capable of nicking DNA in a cell. In embodiments, the endonuclease domain is capable of nicking DNA in a HEK293T cell. In embodiments, an unrepaired nick that undergoes replication in the absence of Rad51 results in increased NHEJ rates at the site of the nick, which can be detected, e.g., by using a Rad51 inhibition assay, e.g., as described in Bothmer et al. (2017) Nat Commun 8:13905 (incorporated by reference herein in its entirety). In embodiments, NHEJ rates are increased above 0-5%. In embodiments, NHEJ rates are increased to 20-70% (e.g., between 30%-60% or 40-50%), e.g., upon Rad51 inhibition.

In some embodiments, the endonuclease domain releases the target after cleavage. In some embodiments, release of the target is indicated indirectly by assessing for multiple turnovers by the enzyme, e.g., as described in Yourik at al. RNA 25(1):35-44 (2019) (incorporated herein by reference in its entirety) and shown in FIG. 2. In some embodiments, the kₑₓₚ of an endonuclease domain is 1×10⁻³-1×10⁻5 min−1 as measured by such methods.

In some embodiments, the endonuclease domain has a catalytic efficiency (k_{cat}/Kₘ) greater than about 1×10⁸ s⁻¹ M⁻¹ in vitro. In embodiments, the endonuclease domain has a catalytic efficiency greater than about 1×10⁵, 1×10⁶, 1×10⁷, or 1×10⁸, s⁻¹ M⁻¹ in vitro. In embodiments, catalytic efficiency is determined as described in Chen et al. (2018) Science 360(6387):436-439 (incorporated herein by reference in its entirety). In some embodiments, the endonuclease domain has a catalytic efficiency (k_{cat}/Kₘ) greater than about 1×10⁸ s⁻¹ M⁻¹ in cells. In embodiments, the endonuclease domain has a catalytic efficiency greater than about 1×10⁵, 1×10⁶, 1×10⁷, or 1×10⁸ s⁻¹ M⁻¹ in cells.

### Heterologous Gene Modifying Polypeptides Comprising Cas Domains

In some embodiments, a heterologous gene modifying polypeptide described herein comprises a Cas domain. In some embodiments, the Cas domain can direct the heterologous gene modifying polypeptide to a target site specified by a gRNA spacer, thereby modifying a target nucleic acid sequence in “cis”. In some embodiments, a heterologous gene modifying polypeptide is fused to a Cas domain. In some embodiments, a heterologous gene modifying polypeptide comprises a CRISPR/Cas domain (also referred to herein as a CRISPR-associated protein). In some embodiments, a CRISPR/Cas domain comprises a protein involved in the clustered regulatory interspaced short palindromic repeat (CRISPR) system, e.g., a Cas protein, and optionally binds a guide RNA, e.g., single guide RNA (sgRNA).

CRISPR systems are adaptive defense systems originally discovered in bacteria and archaea. CRISPR systems use RNA-guided nucleases termed CRISPR-associated or “Cas” endonucleases (e.g., Cas9 or Cpf1) to cleave foreign DNA. For example, in a typical CRISPR-Cas system, an endonuclease is directed to a target nucleotide sequence (e.g., a site in the genome that is to be sequence-edited) by sequence-specific, non-coding “guide RNAs” that target single- or double-stranded DNA sequences. Three classes (I-III) of CRISPR systems have been identified. The class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “spacer” sequence, a typically about 20-nucleotide RNA sequence that corresponds to a target DNA sequence (“protospacer”). In the wild-type system, and in some engineered systems, crRNA also contains a region that binds to the tracrRNA to form a partially double-stranded structure that is cleaved by RNase III, resulting in a crRNA/tracrRNA hybrid molecule. A crRNA/tracrRNA hybrid then directs the Cas endonuclease to recognize and cleave a target DNA sequence. A target DNA sequence is generally adjacent to a “protospacer adjacent motif” (“PAM”) that is specific for a given Cas endonuclease and required for cleavage activity at a target site matching the spacer of the crRNA. CRISPR endonucleases identified from various prokaryotic species have unique PAM sequence requirements, e.g., as listed for exemplary Cas enzymes in Table 7; examples of PAM sequences include 5′-NGG (Streptococcus pyogenes), 5′-NNAGAA (Streptococcus thermophilus CRISPR1), 5′-NGGNG (Streptococcus thermophilus CRISPR3), and 5′-NNNGATT (Neisseria meningiditis). Some endonucleases, e.g., Cas9 endonucleases, are associated with G-rich PAM sites, e.g., 5′-NGG, and perform blunt-end cleaving of the target DNA at a location 3 nucleotides upstream from (5′ from) the PAM site. Another class II CRISPR system includes the type V endonuclease Cpf1, which is smaller than Cas9; examples include AsCpf1 (from Acidaminococcus sp.) and LbCpf1 (from Lachnospiraceae sp.). Cpf1-associated CRISPR arrays are processed into mature crRNAs without the requirement of a tracrRNA; in other words, a Cpf1 system, in some embodiments, comprises only Cpf1 nuclease and a crRNA to cleave a target DNA sequence. Cpf1 endonucleases, are typically associated with T-rich PAM sites, e.g., 5′-TTN. Cpf1 can also recognize a 5′-CTA PAM motif. Cpf1 typically cleaves a target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from a PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e.g., Zetsche et al. (2015) Cell, 163:759-771.

A variety of CRISPR associated (Cas) genes or proteins can be used in the technologies provided by the present disclosure and the choice of Cas protein will depend upon the particular conditions of the method. Specific examples of Cas proteins include class II systems including Cas1, Cas2, Cas3, Cas4, Cas5, Cas6, Cas7, Cas8, Cas9, Cas10, Cpf1, C2C1, or C2C3. In some embodiments, a Cas protein, e.g., a Cas9 protein, may be from any of a variety of prokaryotic species. In some embodiments a particular Cas protein, e.g., a particular Cas9 protein, is selected to recognize a particular protospacer-adjacent motif (PAM) sequence. In some embodiments, a DNA-binding domain or endonuclease domain includes a sequence targeting polypeptide, such as a Cas protein, e.g., Cas9. In certain embodiments a Cas protein, e.g., a Cas9 protein, may be obtained from a bacteria or archaea or synthesized using known methods. In certain embodiments, a Cas protein may be from a gram-positive bacteria or a gram-negative bacteria. In certain embodiments, a Cas protein may be from a Streptococcus (e.g., a S. pyogenes, or a S. thermophilus), a Francisella (e.g., an F. novicida), a Staphylococcus (e.g., an S. aureus), an Acidaminococcus (e.g., an Acidaminococcus sp. BV3L6), a Neisseria (e.g., an N. meningitidis), a Cryptococcus, a Corynebacterium, a Haemophilus, a Eubacterium, a Pasteurella, a Prevotella, a Veillonella, or a Marinobacter.

In some embodiments, a heterologous gene modifying polypeptide may comprise the amino acid sequence of SEQ ID NO: 4000 below, or a sequence having at least 70%, 75%, 80%, 85%, 90%, 95%, 96%, 97%, 98%, 99% identity thereto. In embodiments, the amino acid sequence of SEQ ID NO: 4000 below, or the sequence having at least 70%, 75%, 80%, 85%, 90%, 95%, 96%, 97%, 98%, 99% identity thereto, is positioned at the N-terminal end of the heterologous gene modifying polypeptide. In embodiments, the amino acid sequence of SEQ ID NO: 4000 below, or the sequence having at least 70%, 75%, 80%, 85%, 90%, 95%, 96%, 97%, 98%, 99% identity thereto, is positioned within 1, 2, 3, 4, 5, 6, 7, 8, 9, 10, 15, 20, 25, or 30 amino acids of the N-terminal end of the heterologous gene modifying polypeptide.

Exemplary N-terminal NLS-Cas9 domain
(SEQ ID NO: 4000)
MPAAKRVKLDGGDKKYSIGLDIGTNSVGWAVITDEYKVPSKKFKVLGNTDRHSI
KKNLIGALLFDSGETAEATRLKRTARRRYTRRKNRICYLQEIFSNEMAKVDDSFFHRLEE
SFLVEEDKKHERHPIFGNIVDEVAYHEKYPTIYHLRKKLVDSTDKADLRLIYLALAHMIK
FRGHFLIEGDLNPDNSDVDKLFIQLVQTYNQLFEENPINASGVDAKAILSARLSKSRRLE
NLIAQLPGEKKNGLFGNLIALSLGLTPNFKSNFDLAEDAKLQLSKDTYDDDLDNLLAQIG
DQYADLFLAAKNLSDAILLSDILRVNTEITKAPLSASMIKRYDEHHQDLTLLKALVRQQL
PEKYKEIFFDQSKNGYAGYIDGGASQEEFYKFIKPILEKMDGTEELLVKLNREDLLRKQR
TFDNGSIPHQIHLGELHAILRRQEDFYPFLKDNREKIEKILTFRIPYYVGPLARGNSRFAW
MTRKSEETITPWNFEEVVDKGASAQSFIERMTNFDKNLPNEKVLPKHSLLYEYFTVYNE
LTKVKYVTEGMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMIEERLKTYAHLF
DDKVMKQLKRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVMGRHKPENIVI
EMARENQTTQKGQKNSRERMKRIEEGIKELGSQILKEHPVENTQLQNEKLYLYYLQNGR
DMYVDQELDINRLSDYDVDHIVPQSFLKDDSIDNKVLTRSDKARGKSDNVPSEEVVKK
MKNYWRQLLNAKLITQRKFDNLTKAERGGLSELDKAGFIKRQLVETRQITKHVAQILDS
RMNTKYDENDKLIREVKVITLKSKLVSDFRKDFQFYKVREINNYHHAHDAYLNAVVGT
ALIKKYPKLESEFVYGDYKVYDVRKMIAKSEQEIGKATAKYFFYSNIMNFFKTEITLANG
EIRKRPLIETNGETGEIVWDKGRDFATVRKVLSMPQVNIVKKTEVQTGGFSKESILPKRN
SDKLIARKKDWDPKKYGGFDSPTVAYSVLVVAKVEKGKSKKLKSVKELLGITIMERSSF
EKNPIDFLEAKGYKEVKKDLIIKLPKYSLFELENGRKRMLASAGELQKGNELALPSKYV
NFLYLASHYEKLKGSPEDNEQKQLFVEQHKHYLDEIIEQISEFSKRVILADANLDKVLSA
YNKHRDKPIREQAENIIHLFTLTNLGAPAAFKYFDTTIDRKRYTSTKEVLDATLIHQSITG
LYETRIDLSQLGGDGG

In some embodiments, a heterologous gene modifying polypeptide may comprise the amino acid sequence of SEQ ID NO: 4001 below, or a sequence having at least 70%, 75%, 80%, 85%, 90%, 95%, 96%, 97%, 98%, 99% identity thereto. In embodiments, the amino acid sequence of SEQ ID NO: 4001 below, or the sequence having at least 70%, 75%, 80%, 85%, 90%, 95%, 96%, 97%, 98%, 99% identity thereto, is positioned at the C-terminal end of the heterologous gene modifying polypeptide. In embodiments, the amino acid sequence of SEQ ID NO: 4001 below, or the sequence having at least 70%, 75%, 80%, 85%, 90%, 95%, 96%, 97%, 98%, 99% identity thereto, is positioned within 1, 2, 3, 4, 5, 6, 7, 8, 9, 10, 15, 20, 25, or 30 amino acids of the C-terminal end of the heterologous gene modifying polypeptide.

Exemplary C-terminal sequence comprising an NLS
(SEQ ID NO: 4001)
AGKRTADGSEFEKRTADGSEFESPKKKAKVE
Exemplary benchmarking sequence
(SEQ ID NO: 4002)
MPAAKRVKLDGGDKKYSIGLDIGTNSVGWAVITDEYKVPSKKFKVLGNTDRHSI
KKNLIGALLFDSGETAEATRLKRTARRRYTRRKNRICYLQEIFSNEMAKVDDSFFHRLEE
SFLVEEDKKHERHPIFGNIVDEVAYHEKYPTIYHLRKKLVDSTDKADLRLIYLALAHMIK
FRGHFLIEGDLNPDNSDVDKLFIQLVQTYNQLFEENPINASGVDAKAILSARLSKSRRLE
NLIAQLPGEKKNGLFGNLIALSLGLTPNFKSNFDLAEDAKLQLSKDTYDDDLDNLLAQIG
DQYADLFLAAKNLSDAILLSDILRVNTEITKAPLSASMIKRYDEHHQDLTLLKALVRQQL
PEKYKEIFFDQSKNGYAGYIDGGASQEEFYKFIKPILEKMDGTEELLVKLNREDLLRKQR
TFDNGSIPHQIHLGELHAILRRQEDFYPFLKDNREKIEKILTFRIPYYVGPLARGNSRFAW
MTRKSEETITPWNFEEVVDKGASAQSFIERMTNFDKNLPNEKVLPKHSLLYEYFTVYNE
LTKVKYVTEGMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMIEERLKTYAHLF
DDKVMKQLKRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVMGRHKPENIVI
EMARENQTTQKGQKNSRERMKRIEEGIKELGSQILKEHPVENTQLQNEKLYLYYLQNGR
DMYVDQELDINRLSDYDVDHIVPQSFLKDDSIDNKVLTRSDKARGKSDNVPSEEVVKK
MKNYWRQLLNAKLITQRKFDNLTKAERGGLSELDKAGFIKRQLVETRQITKHVAQILDS
RMNTKYDENDKLIREVKVITLKSKLVSDFRKDFQFYKVREINNYHHAHDAYLNAVVGT
ALIKKYPKLESEFVYGDYKVYDVRKMIAKSEQEIGKATAKYFFYSNIMNFFKTEITLANG
EIRKRPLIETNGETGEIVWDKGRDFATVRKVLSMPQVNIVKKTEVQTGGFSKESILPKRN
SDKLIARKKDWDPKKYGGFDSPTVAYSVLVVAKVEKGKSKKLKSVKELLGITIMERSSF
EKNPIDFLEAKGYKEVKKDLIIKLPKYSLFELENGRKRMLASAGELQKGNELALPSKYV
NFLYLASHYEKLKGSPEDNEQKQLFVEQHKHYLDEIIEQISEFSKRVILADANLDKVLSA
YNKHRDKPIREQAENIIHLFTLTNLGAPAAFKYFDTTIDRKRYTSTKEVLDATLIHQSITG
LYETRIDLSQLGGDGGSGGSSGGSSGSETPGTSESATPESSGGSSGGSSGGTLNIEDEYRL
HETSKEPDVSLGSTWLSDFPQAWAETGGMGLAVRQAPLIIPLKATSTPVSIKQYPMSQE
ARLGIKPHIQRLLDQGILVPCQSPWNTPLLPVKKPGTNDYRPVQDLREVNKRVEDIHPTV
PNPYNLLSGLPPSHQWYTVLDLKDAFFCLRLHPTSQPLFAFEWRDPEMGISGQLTWTRL
PQGFKNSPTLFNEALHRDLADFRIQHPDLILLQYVDDLLLAATSELDCQQGTRALLQTLG
NLGYRASAKKAQICQKQVKYLGYLLKEGQRWLTEARKETVMGQPTPKTPRQLREFLG
KAGFCRLFIPGFAEMAAPLYPLTKPGTLFNWGPDQQKAYQEIKQALLTAPALGLPDLTK
PFELFVDEKQGYAKGVLTQKLGPWRRPVAYLSKKLDPVAAGWPPCLRMVAAIAVLTK
DAGKLTMGQPLVILAPHAVEALVKQPPDRWLSNARMTHYQALLLDTDRVQFGPVVAL
NPATLLPLPEEGLQHNCLDILAEAHGTRPDLTDQPLPDADHTWYTDGSSLLQEGQRKAG
AAVTTETEVIWAKALPAGTSAQRAELIALTQALKMAEGKKLNVYTDSRYAFATAHIHG
EIYRRRGWLTSEGKEIKNKDEILALLKALFLPKRLSIIHCPGHQKGHSAEARGNRMADQA
ARKAAITETPDTSTLLIENSSPSGGSKRTADGSEFEAGKRTADGSEFEKRTADGSEFESPK
KKAKVE

In some embodiments, a heterologous gene modifying polypeptide may comprise a Cas domain as listed in Table 7 or 8, or a functional fragment thereof, or a sequence having at least 70%, 75%, 80%, 85%, 90%, 95%, 96%, 97%, 98%, 99% identity thereto.

TABLE 7
CRISPR/Cas Proteins, Species, and Mutations

| | | | # of | | Mutations to alter | Mutations to make |
|---|---|---|---|---|---|---|
| Name | Enzyme | Species | AAs | PAM | PAM recognition | catalytically dead |
| FnCas9 | Cas9 | Francisella | 1629 | 5′-NGG-3′ | Wt | D11A/H969A/N995A |
| | | novicida | | | | |
| FnCas9 | Cas9 | Francisella | 1629 | 5′-YG-3′ | E1369R/E1449H/R1556A | D11A/H969A/N995A |
| RHA | | novicida | | | | |
| SaCas9 | Cas9 | Staphylococcus | 1053 | 5′-NNGRRT-3′ | Wt | D10A/H557A |
| | | aureus | | | | |
| SaCas9 | Cas9 | Staphylococcus | 1053 | 5′-NNNRRT-3′ | E782K/N968K/R1015H | D10A/H557A |
| KKH | | aureus | | | | |
| SpCas9 | Cas9 | Streptococcus | 1368 | 5′-NGG-3′ | Wt | D10A/D839A/H840A/N863A |
| | | pyogenes | | | | |
| SpCas9 | Cas9 | Streptococcus | 1368 | 5′-NGA-3′ | D1135V/R1335Q/T1337R | D10A/D839A/H840A/N863A |
| VQR | | pyogenes | | | | |
| AsCpf1 | Cpf1 | Acidaminococcus | 1307 | 5′-TYCV-3′ | S542R/K607R | E993A |
| RR | | sp. BV3L6 | | | | |
| AsCpf1 | Cpf1 | Acidaminococcus | 1307 | 5′-TATV-3′ | S542R/K548V/N552R | E993A |
| RVR | | sp. BV3L6 | | | | |
| FnCpf1 | Cpf1 | Francisella | 1300 | 5′-NTTN-3′ | Wt | D917A/E1006A/D1255A |
| | | novicida | | | | |
| NmCas9 | Cas9 | Neisseria | 1082 | 5′-NNNGATT-3′ | Wt | D16A/D587A/H588A/N611A |
| | | meningitidis | | | | |

TABLE 8A
Amino Acid Sequences of CRISPR/Cas Proteins, Species, and Mutations

| | Parental | | SEQ ID | | | |
|---|---|---|---|---|---|---|
| Variant | Host(s) | Protein Sequence | NO: | (HNH) | (HNH) | (RuvC) |
| Nme2Cas9 | Neisseria | MAAFKPNPINYILGLDIGIASVGWAMVEIDEEENPIRLIDLGVR | 9,001 | N611A | H588A | D16A |
| | meningitidis | VFERAEVPKTGDSLAMARRLARSVRRLTRRRAHRLLRARRLL | | | | |
| | | KREGVLQAADFDENGLIKSLPNTPWQLRAAALDRKLTPLEWS | | | | |
| | | AVLLHLIKHRGYLSQRKNEGETADKELGALLKGVANNAHAL | | | | |
| | | QTGDFRTPAELALNKFEKESGHIRNQRGDYSHTFSRKDLQAE | | | | |
| | | LILLFEKQKEFGNPHVSGGLKEGIETLLMTQRPALSGDAVQK | | | | |
| | | MLGHCTFEPAEPKAAKNTYTAERFIWLTKLNNLRILEQGSERP | | | | |
| | | LTDTERATLMDEPYRKSKLTYAQARKLLGLEDTAFFKGLRY | | | | |
| | | GKDNAEASTLMEMKAYHAISRALEKEGLKDKKSPLNLSSELQ | | | | |
| | | DEIGTAFSLFKTDEDITGRLKDRVQPEILEALLKHISFDKFVQIS | | | | |
| | | LKALRRIVPLMEQGKRYDEACAEIYGDHYGKKNTEEKIYLPPI | | | | |
| | | PADEIRNPVVLRALSQARKVINGVVRRYGSPARIHIETAREVG | | | | |
| | | KSFKDRKEIEKRQEENRKDREKAAAKFREYFPNFVGEPKSKDI | | | | |
| | | LKLRLYEQQHGKCLYSGKEINLVRLNEKGYVEIDHALPFSRT | | | | |
| | | WDDSFNNKVLVLGSENQNKGNQTPYEYFNGKDNSREWQEF | | | | |
| | | KARVETSRFPRSKKQRILLQKFDEDGFKECNLNDTRYVNRFL | | | | |
| | | CQFVADHILLTGKGKRRVFASNGQITNLLRGFWGLRKVRAEN | | | | |
| | | DRHHALDAVVVACSTVAMQQKITRFVRYKEMNAFDGKTIDK | | | | |
| | | ETGKVLHQKTHFPQPWEFFAQEVMIRVFGKPDGKPEFEEADT | | | | |
| | | PEKLRTLLAEKLSSRPEAVHEYVTPLFVSRAPNRKMSGAHKD | | | | |
| | | TLRSAKRFVKHNEKISVKRVWLTEIKLADLENMVNYKNGREI | | | | |
| | | ELYEALKARLEAYGGNAKQAFDPKDNPFYKKGGQLVKAVR | | | | |
| | | VEKTQESGVLLNKKNAYTIADNGDMVRVDVFCKVDKKGKN | | | | |
| | | QYFIVPIYAWQVAENILPDIDCKGYRIDDSYTFCFSLHKYDLIA | | | | |
| | | FQKDEKSKVEFAYYINCDSSNGRFYLAWHDKGSKEQQFRIST | | | | |
| | | QNLVLIQKYQVNELGKEIRPCRLKKRPPVR | | | | |
| PpnCas9 | Pasteurella | MQNNPLNYILGLDLGIASIGWAVVEIDEESSPIRLIDVGVRTFE | 9,002 | N605A | H582A | D13A |
| | pneumotropica | RAEVAKTGESLALSRRLARSSRRLIKRRAERLKKAKRLLKAE | | | | |
| | | KILHSIDEKLPINVWQLRVKGLKEKLERQEWAAVLLHLSKHR | | | | |
| | | GYLSQRKNEGKSDNKELGALLSGIASNHQMLQSSEYRTPAEI | | | | |
| | | AVKKFQVEEGHIRNQRGSYTHTFSRLDLLAEMELLFQRQAEL | | | | |
| | | GNSYTSTTLLENLTALLMWQKPALAGDAILKMLGKCTFEPSE | | | | |
| | | YKAAKNSYSAERFVWLTKLNNLRILENGTERALNDNERFALL | | | | |
| | | EQPYEKSKLTYAQVRAMLALSDNAIFKGVRYLGEDKKTVES | | | | |
| | | KTTLIEMKFYHQIRKTLGSAELKKEWNELKGNSDLLDEIGTAF | | | | |
| | | SLYKTDDDICRYLEGKLPERVLNALLENLNFDKFIQLSLKALH | | | | |
| | | QILPLMLQGQRYDEAVSAIYGDHYGKKSTETTRLLPTIPADEI | | | | |
| | | RNPVVLRTLTQARKVINAVVRLYGSPARIHIETAREVGKSYQ | | | | |
| | | DRKKLEKQQEDNRKQRESAVKKFKEMFPHFVGEPKGKDILK | | | | |
| | | MRLYELQQAKCLYSGKSLELHRLLEKGYVEVDHALPFSRTW | | | | |
| | | DDSFNNKVLVLANENQNKGNLTPYEWLDGKNNSERWQHFV | | | | |
| | | VRVQTSGFSYAKKQRILNHKLDEKGFIERNLNDTRYVARFLC | | | | |
| | | NFIADNMLLVGKGKRNVFASNGQITALLRHRWGLQKVREQN | | | | |
| | | DRHHALDAVVVACSTVAMQQKITRFVRYNEGNVFSGERIDR | | | | |
| | | ETGEIIPLHFPSPWAFFKENVEIRIFSENPKLELENRLPDYPQYN | | | | |
| | | HEWVQPLFVSRMPTRKMTGQGHMETVKSAKRLNEGLSVLK | | | | |
| | | VPLTQLKLSDLERMVNRDREIALYESLKARLEQFGNDPAKAF | | | | |
| | | AEPFYKKGGALVKAVRLEQTQKSGVLVRDGNGVADNASMV | | | | |
| | | RVDVFTKGGKYFLVPIYTWQVAKGILPNRAATQGKDENDWD | | | | |
| | | IMDEMATFQFSLCQNDLIKLVTKKKTIFGYFNGLNRATSNINI | | | | |
| | | KEHDLDKSKGKLGIYLEVGVKLAISLEKYQVDELGKNIRPCR | | | | |
| | | PTKRQHVR | | | | |
| SauCas9 | Staphylococcus | MKRNYILGLDIGITSVGYGIIDYETRDVIDAGVRLFKEANVEN | 9,003 | N580A | H557A | D10A |
| SauCas9- | aureus | NEGRRSKRGARRLKRRRRHRIQRVKKLLFDYNLLTDHSELSG | | | | |
| | | INPYEARVKGLSQKLSEEEFSAALLHLAKRRGVHNVNEVEED | | | | |
| | | TGNELSTKEQISRNSKALEEKYVAELQLERLKKDGEVRGSINR | | | | |
| | | FKTSDYVKEAKQLLKVQKAYHQLDQSFIDTYIDLLETRRTYY | | | | |
| | | EGPGEGSPFGWKDIKEWYEMLMGHCTYFPEELRSVKYAYNA | | | | |
| | | DLYNALNDLNNLVITRDENEKLEYYEKFQIIENVFKQKKKPTL | | | | |
| | | KQIAKEILVNEEDIKGYRVTSTGKPEFTNLKVYHDIKDITARK | | | | |
| | | EIIENAELLDQIAKILTIYQSSEDIQEELTNLNSELTQEEIEQISN | | | | |
| | | LKGYTGTHNLSLKAINLILDELWHTNDNQIAIFNRLKLVPKKV | | | | |
| | | DLSQQKEIPTTLVDDFILSPVVKRSFIQSIKVINAIIKKYGLPNDI | | | | |
| | | IIELAREKNSKDAQKMINEMQKRNRQTNERIEEIIRTTGKENA | | | | |
| | | KYLIEKIKLHDMQEGKCLYSLEAIPLEDLLNNPFNYEVDHIIPR | | | | |
| | | SVSFDNSFNNKVLVKQEENSKKGNRTPFQYLSSSDSKISYETF | | | | |
| | | KKHILNLAKGKGRISKTKKEYLLEERDINRFSVQKDFINRNLV | | | | |
| | | DTRYATRGLMNLLRSYFRVNNLDVKVKSINGGFTSFLRRKW | | | | |
| | | KFKKERNKGYKHHAEDALIIANADFIFKEWKKLDKAKKVME | | | | |
| | | NQMFEEKQAESMPEIETEQEYKEIFITPHQIKHIKDFKDYKYSH | | | | |
| | | RVDKKPNRELINDTLYSTRKDDKGNTLIVNNLNGLYDKDND | | | | |
| | | KLKKLINKSPEKLLMYHHDPQTYQKLKLIMEQYGDEKNPLY | | | | |
| | | KYYEETGNYLTKYSKKDNGPVIKKIKYYGNKLNAHLDITDD | | | | |
| | | YPNSRNKVVKLSLKPYRFDVYLDNGVYKFVTVKNLDVIKKE | | | | |
| | | NYYEVNSKCYEEAKKLKKISNQAEFIASFYNNDLIKINGELYR | | | | |
| | | VIGVNNDLLNRIEVNMIDITYREYLENMNDKRPPRIIKTIASKT | | | | |
| | | QSIKKYSTDILGNLYEVKSKKHPQIIKKG | | | | |
| KKH | Staphylococcus | MKRNYILGLDIGITSVGYGIIDYETRDVIDAGVRLFKEANVEN | 9,004 | N580A | H557A | D10A |
| | aureus | NEGRRSKRGARRLKRRRRHRIQRVKKLLFDYNLLTDHSELSG | | | | |
| | | INPYEARVKGLSQKLSEEEFSAALLHLAKRRGVHNVNEVEED | | | | |
| | | TGNELSTKEQISRNSKALEEKYVAELQLERLKKDGEVRGSINR | | | | |
| | | FKTSDYVKEAKQLLKVQKAYHQLDQSFIDTYIDLLETRRTYY | | | | |
| | | EGPGEGSPFGWKDIKEWYEMLMGHCTYFPEELRSVKYAYNA | | | | |
| | | DLYNALNDLNNLVITRDENEKLEYYEKFQIIENVFKQKKKPTL | | | | |
| | | KQIAKEILVNEEDIKGYRVTSTGKPEFTNLKVYHDIKDITARK | | | | |
| | | EIIENAELLDQIAKILTIYQSSEDIQEELTNLNSELTQEEIEQISN | | | | |
| | | LKGYTGTHNLSLKAINLILDELWHTNDNQIAIFNRLKLVPKKV | | | | |
| | | DLSQQKEIPTTLVDDFILSPVVKRSFIQSIKVINAIIKKYGLPNDI | | | | |
| | | IIELAREKNSKDAQKMINEMQKRNRQTNERIEEIIRTTGKENA | | | | |
| | | KYLIEKIKLHDMQEGKCLYSLEAIPLEDLLNNPFNYEVDHIIPR | | | | |
| | | SVSFDNSFNNKVLVKQEENSKKGNRTPFQYLSSSDSKISYETF | | | | |
| | | KKHILNLAKGKGRISKTKKEYLLEERDINRFSVQKDFINRNLV | | | | |
| | | DTRYATRGLMNLLRSYFRVNNLDVKVKSINGGFTSFLRRKW | | | | |
| | | KFKKERNKGYKHHAEDALIIANADFIFKEWKKLDKAKKVME | | | | |
| | | NQMFEEKQAESMPEIETEQEYKEIFITPHQIKHIKDFKDYKYSH | | | | |
| | | RVDKKPNRKLINDTLYSTRKDDKGNTLIVNNLNGLYDKDND | | | | |
| | | KLKKLINKSPEKLLMYHHDPQTYQKLKLIMEQYGDEKNPLY | | | | |
| | | KYYEETGNYLTKYSKKDNGPVIKKIKYYGNKLNAHLDITDD | | | | |
| | | YPNSRNKVVKLSLKPYRFDVYLDNGVYKFVTVKNLDVIKKE | | | | |
| | | NYYEVNSKCYEEAKKLKKISNQAEFIASFYKNDLIKINGELYR | | | | |
| | | VIGVNNDLLNRIEVNMIDITYREYLENMNDKRPPHIIKTIASKT | | | | |
| | | QSIKKYSTDILGNLYEVKSKKHPQIIKKG | | | | |
| SauriCas9 | Staphylococcus | MQENQQKQNYILGLDIGITSVGYGLIDSKTREVIDAGVRLFPE | 9,005 | N588A | H565A | D15A |
| | auricularis | ADSENNSNRRSKRGARRLKRRRIHRLNRVKDLLADYQMIDL | | | | |
| | | NNVPKSTDPYTIRVKGLREPLTKEEFAIALLHIAKRRGLHNISV | | | | |
| | | SMGDEEQDNELSTKQQLQKNAQQLQDKYVCELQLERLTNIN | | | | |
| | | KVRGEKNRFKTEDFVKEVKQLCETQRQYHNIDDQFIQQYIDL | | | | |
| | | VSTRREYFEGPGNGSPYGWDGDLLKWYEKLMGRCTYFPEEL | | | | |
| | | RSVKYAYSADLFNALNDLNNLVVTRDDNPKLEYYEKYHIIEN | | | | |
| | | VFKQKKNPTLKQIAKEIGVQDYDIRGYRITKSGKPQFTSFKLY | | | | |
| | | HDLKNIFEQAKYLEDVEMLDEIAKILTIYQDEISIKKALDQLPE | | | | |
| | | LLTESEKSQIAQLTGYTGTHRLSLKCIHIVIDELWESPENQMEI | | | | |
| | | FTRLNLKPKKVEMSEIDSIPTTLVDEFILSPVVKRAFIQSIKVIN | | | | |
| | | AVINRFGLPEDIIIELAREKNSKDRRKFINKLQKQNEATRKKIE | | | | |
| | | QLLAKYGNTNAKYMIEKIKLHDMQEGKCLYSLEAIPLEDLLS | | | | |
| | | NPTHYEVDHIIPRSVSFDNSLNNKVLVKQSENSKKGNRTPYQ | | | | |
| | | YLSSNESKISYNQFKQHILNLSKAKDRISKKKRDMLLEERDIN | | | | |
| | | KFEVQKEFINRNLVDTRYATRELSNLLKTYFSTHDYAVKVKT | | | | |
| | | INGGFTNHLRKVWDFKKHRNHGYKHHAEDALVIANADFLFK | | | | |
| | | THKALRRTDKILEQPGLEVNDTTVKVDTEEKYQELFETPKQV | | | | |
| | | KNIKQFRDFKYSHRVDKKPNRQLINDTLYSTREIDGETYVVQ | | | | |
| | | TLKDLYAKDNEKVKKLFTERPQKILMYQHDPKTFEKLMTILN | | | | |
| | | QYAEAKNPLAAYYEDKGEYVTKYAKKGNGPAIHKIKYIDKK | | | | |
| | | LGSYLDVSNKYPETQNKLVKLSLKSFRFDIYKCEQGYKMVSI | | | | |
| | | GYLDVLKKDNYYYIPKDKYEAEKQKKKIKESDLFVGSFYYN | | | | |
| | | DLIMYEDELFRVIGVNSDINNLVELNMVDITYKDFCEVNNVT | | | | |
| | | GEKRIKKTIGKRVVLIEKYTTDILGNLYKTPLPKKPQLIFKRGE | | | | |
| | | L | | | | |
| SauriCas9- | Staphylococcus | MQENQQKQNYILGLDIGITSVGYGLIDSKTREVIDAGVRLFPE | 9,006 | N588A | H565A | D15A |
| KKH | auricularis | ADSENNSNRRSKRGARRLKRRRIHRLNRVKDLLADYQMIDL | | | | |
| | | NNVPKSTDPYTIRVKGLREPLTKEEFAIALLHIAKRRGLHNISV | | | | |
| | | SMGDEEQDNELSTKQQLQKNAQQLQDKYVCELQLERLTNIN | | | | |
| | | KVRGEKNRFKTEDFVKEVKQLCETQRQYHNIDDQFIQQYIDL | | | | |
| | | VSTRREYFEGPGNGSPYGWDGDLLKWYEKLMGRCTYFPEEL | | | | |
| | | RSVKYAYSADLFNALNDLNNLVVTRDDNPKLEYYEKYHIIEN | | | | |
| | | VFKQKKNPTLKQIAKEIGVQDYDIRGYRITKSGKPQFTSFKLY | | | | |
| | | HDLKNIFEQAKYLEDVEMLDEIAKILTIYQDEISIKKALDQLPE | | | | |
| | | LLTESEKSQIAQLTGYTGTHRLSLKCIHIVIDELWESPENQMEI | | | | |
| | | FTRLNLKPKKVEMSEIDSIPTTLVDEFILSPVVKRAFIQSIKVIN | | | | |
| | | AVINRFGLPEDIIIELAREKNSKDRRKFINKLQKQNEATRKKIE | | | | |
| | | QLLAKYGNTNAKYMIEKIKLHDMQEGKCLYSLEAIPLEDLLS | | | | |
| | | NPTHYEVDHIIPRSVSFDNSLNNKVLVKQSENSKKGNRTPYQ | | | | |
| | | YLSSNESKISYNQFKQHILNLSKAKDRISKKKRDMLLEERDIN | | | | |
| | | KFEVQKEFINRNLVDTRYATRELSNLLKTYFSTHDYAVKVKT | | | | |
| | | INGGFTNHLRKVWDFKKHRNHGYKHHAEDALVIANADFLFK | | | | |
| | | THKALRRTDKILEQPGLEVNDTTVKVDTEEKYQELFETPKQV | | | | |
| | | KNIKQFRDFKYSHRVDKKPNRKLINDTLYSTREIDGETYVVQ | | | | |
| | | TLKDLYAKDNEKVKKLFTERPQKILMYQHDPKTFEKLMTILN | | | | |
| | | QYAEAKNPLAAYYEDKGEYVTKYAKKGNGPAIHKIKYIDKK | | | | |
| | | LGSYLDVSNKYPETQNKLVKLSLKSFRFDIYKCEQGYKMVSI | | | | |
| | | GYLDVLKKDNYYYIPKDKYEAEKQKKKIKESDLFVGSFYKN | | | | |
| | | DLIMYEDELFRVIGVNSDINNLVELNMVDITYKDFCEVNNVT | | | | |
| | | GEKHIKKTIGKRVVLIEKYTTDILGNLYKTPLPKKPQLIFKRGE | | | | |
| | | L | | | | |
| ScaCas9- | Streptococcus | MEKKYSIGLDIGTNSVGWAVITDDYKVPSKKFKVLGNTNRKS | 9,007 | N872A | H849A | D10A |
| Sc++ | canis | IKKNLMGALLFDSGETAEATRLKRTARRRYTRRKNRIRYLQEI | | | | |
| | | FANEMAKLDDSFFQRLEESFLVEEDKKNERHPIFGNLADEVA | | | | |
| | | YHRNYPTIYHLRKKLADSPEKADLRLIYLALAHIIKFRGHFLIE | | | | |
| | | GKLNAENSDVAKLFYQLIQTYNQLFEESPLDEIEVDAKGILSA | | | | |
| | | RLSKSKRLEKLIAVFPNEKKNGLFGNIIALALGLTPNFKSNFDL | | | | |
| | | TEDAKLQLSKDTYDDDLDELLGQIGDQYADLFSAAKNLSDAI | | | | |
| | | LLSDILRSNSEVTKAPLSASMVKRYDEHHQDLALLKTLVRQQ | | | | |
| | | FPEKYAEIFKDDTKNGYAGYVGADKKLRKRSGKLATEEEFY | | | | |
| | | KFIKPILEKMDGAEELLAKLNRDDLLRKQRTFDNGSIPHQIHL | | | | |
| | | KELHAILRRQEEFYPFLKENREKIEKILTFRIPYYVGPLARGNS | | | | |
| | | RFAWLTRKSEEAITPWNFEEVVDKGASAQSFIERMTNFDEQL | | | | |
| | | PNKKVLPKHSLLYEYFTVYNELTKVKYVTERMRKPEFLSGEQ | | | | |
| | | KKAIVDLLFKTNRKVTVKQLKEDYFKKIECFDSVEIIGVEDRF | | | | |
| | | NASLGTYHDLLKIIKDKDFLDNEENEDILEDIVLTLTLFEDRE | | | | |
| | | MIEERLKTYAHLFDDKVMKQLKRRHYTGWGRLSRKMINGIR | | | | |
| | | DKQSGKTILDFLKSDGFSNRNFMQLIHDDSLTFKEEIEKAQVS | | | | |
| | | GQGDSLHEQIADLAGSPAIKKGILQTVKIVDELVKVMGHKPE | | | | |
| | | NIVIEMARENQTTTKGLQQSRERKKRIEEGIKELESQILKENPV | | | | |
| | | ENTQLQNEKLYLYYLQNGRDMYVDQELDINRLSDYDVDHIV | | | | |
| | | PQSFIKDDSIDNKVLTRSVENRGKSDNVPSEEVVKKMKNYWR | | | | |
| | | QLLNAKLITQRKFDNLTKAERGGLSEADKAGFIKRQLVETRQI | | | | |
| | | TKHVARILDSRMNTKRDKNDKPIREVKVITLKSKLVSDFRKD | | | | |
| | | FQLYKVRDINNYHHAHDAYLNAVVGTALIKKYPKLESEFVY | | | | |
| | | GDYKVYDVRKMIAKSEQEIGKATAKRFFYSNIMNFFKTEVKL | | | | |
| | | ANGEIRKRPLIETNGETGEVVWNKEKDFATVRKVLAMPQVNI | | | | |
| | | VKKTEVQTGGFSKESILSKRESAKLIPRKKGWDTRKYGGFGS | | | | |
| | | PTVAYSILVVAKVEKGKAKKLKSVKVLVGITIMEKGSYEKDP | | | | |
| | | IGFLEAKGYKDIKKELIFKLPKYSLFELENGRRRMLASAKELQ | | | | |
| | | KANELVLPQHLVRLLYYTQNISATTGSNNLGYIEQHREEFKEI | | | | |
| | | FEKIIDFSEKYILKNKVNSNLKSSFDEQFAVSDSILLSNSFVSLL | | | | |
| | | KYTSFGASGGFTFLDLDVKQGRLRYQTVTEVLDATLIYQSITG | | | | |
| | | LYETRTDLSQLGGD | | | | |
| SpyCas9 | Streptococcus | MDKKYSIGLDIGTNSVGWAVITDEYKVPSKKFKVLGNTDRHS | 9,008 | N863A | H840A | D10A |
| | pyogenes | IKKNLIGALLFDSGETAEATRLKRTARRRYTRRKNRICYLQEIF | | | | |
| | | SNEMAKVDDSFFHRLEESFLVEEDKKHERHPIFGNIVDEVAY | | | | |
| | | HEKYPTIYHLRKKLVDSTDKADLRLIYLALAHMIKFRGHFLIE | | | | |
| | | GDLNPDNSDVDKLFIQLVQTYNQLFEENPINASGVDAKAILSA | | | | |
| | | RLSKSRRLENLIAQLPGEKKNGLFGNLIALSLGLTPNFKSNFDL | | | | |
| | | AEDAKLQLSKDTYDDDLDNLLAQIGDQYADLFLAAKNLSDA | | | | |
| | | ILLSDILRVNTEITKAPLSASMIKRYDEHHQDLTLLKALVRQQ | | | | |
| | | LPEKYKEIFFDQSKNGYAGYIDGGASQEEFYKFIKPILEKMDG | | | | |
| | | TEELLVKLNREDLLRKQRTFDNGSIPHQIHLGELHAILRRQED | | | | |
| | | FYPFLKDNREKIEKILTFRIPYYVGPLARGNSRFAWMTRKSEE | | | | |
| | | TITPWNFEEVVDKGASAQSFIERMTNFDKNLPNEKVLPKHSLL | | | | |
| | | YEYFTVYNELTKVKYVTEGMRKPAFLSGEQKKAIVDLLFKTN | | | | |
| | | RKVTVKQLKEDYFKKIECFDSVEISGVEDRFNASLGTYHDLL | | | | |
| | | KIIKDKDFLDNEENEDILEDIVLTLTLFEDREMIEERLKTYAHL | | | | |
| | | FDDKVMKQLKRRRYTGWGRLSRKLINGIRDKQSGKTILDFLK | | | | |
| | | SDGFANRNFMQLIHDDSLTFKEDIQKAQVSGQGDSLHEHIAN | | | | |
| | | LAGSPAIKKGILQTVKVVDELVKVMGRHKPENIVIEMARENQ | | | | |
| | | TTQKGQKNSRERMKRIEEGIKELGSQILKEHPVENTQLQNEKL | | | | |
| | | YLYYLQNGRDMYVDQELDINRLSDYDVDHIVPQSFLKDDSID | | | | |
| | | NKVLTRSDKNRGKSDNVPSEEVVKKMKNYWRQLLNAKLITQ | | | | |
| | | RKFDNLTKAERGGLSELDKAGFIKRQLVETRQITKHVAQILDS | | | | |
| | | RMNTKYDENDKLIREVKVITLKSKLVSDFRKDFQFYKVREIN | | | | |
| | | NYHHAHDAYLNAVVGTALIKKYPKLESEFVYGDYKVYDVR | | | | |
| | | KMIAKSEQEIGKATAKYFFYSNIMNFFKTEITLANGEIRKRPLI | | | | |
| | | ETNGETGEIVWDKGRDFATVRKVLSMPQVNIVKKTEVQTGG | | | | |
| | | FSKESILPKRNSDKLIARKKDWDPKKYGGFDSPTVAYSVLVV | | | | |
| | | AKVEKGKSKKLKSVKELLGITIMERSSFEKNPIDFLEAKGYKE | | | | |
| | | VKKDLIIKLPKYSLFELENGRKRMLASAGELQKGNELALPSK | | | | |
| | | YVNFLYLASHYEKLKGSPEDNEQKQLFVEQHKHYLDEIIEQIS | | | | |
| | | EFSKRVILADANLDKVLSAYNKHRDKPIREQAENIIHLFTLTN | | | | |
| | | LGAPAAFKYFDTTIDRKRYTSTKEVLDATLIHQSITGLYETRID | | | | |
| | | LSQLGGD | | | | |
| SpyCas9- | Streptococcus | MDKKYSIGLDIGTNSVGWAVITDEYKVPSKKFKVLGNTDRHS | 9,009 | N863A | H840A | D10A |
| NG | pyogenes | IKKNLIGALLFDSGETAEATRLKRTARRRYTRRKNRICYLQEIF | | | | |
| | | SNEMAKVDDSFFHRLEESFLVEEDKKHERHPIFGNIVDEVAY | | | | |
| | | HEKYPTIYHLRKKLVDSTDKADLRLIYLALAHMIKFRGHFLIE | | | | |
| | | GDLNPDNSDVDKLFIQLVQTYNQLFEENPINASGVDAKAILSA | | | | |
| | | RLSKSRRLENLIAQLPGEKKNGLFGNLIALSLGLTPNFKSNFDL | | | | |
| | | AEDAKLQLSKDTYDDDLDNLLAQIGDQYADLFLAAKNLSDA | | | | |
| | | ILLSDILRVNTEITKAPLSASMIKRYDEHHQDLTLLKALVRQQ | | | | |
| | | LPEKYKEIFFDQSKNGYAGYIDGGASQEEFYKFIKPILEKMDG | | | | |
| | | TEELLVKLNREDLLRKQRTFDNGSIPHQIHLGELHAILRRQED | | | | |
| | | FYPFLKDNREKIEKILTFRIPYYVGPLARGNSRFAWMTRKSEE | | | | |
| | | TITPWNFEEVVDKGASAQSFIERMTNFDKNLPNEKVLPKHSLL | | | | |
| | | YEYFTVYNELTKVKYVTEGMRKPAFLSGEQKKAIVDLLFKTN | | | | |
| | | RKVTVKQLKEDYFKKIECFDSVEISGVEDRFNASLGTYHDLL | | | | |
| | | KIIKDKDFLDNEENEDILEDIVLTLTLFEDREMIEERLKTYAHL | | | | |
| | | FDDKVMKQLKRRRYTGWGRLSRKLINGIRDKQSGKTILDFLK | | | | |
| | | SDGFANRNFMQLIHDDSLTFKEDIQKAQVSGQGDSLHEHIAN | | | | |
| | | LAGSPAIKKGILQTVKVVDELVKVMGRHKPENIVIEMARENQ | | | | |
| | | TTQKGQKNSRERMKRIEEGIKELGSQILKEHPVENTQLQNEKL | | | | |
| | | YLYYLQNGRDMYVDQELDINRLSDYDVDHIVPQSFLKDDSID | | | | |
| | | NKVLTRSDKNRGKSDNVPSEEVVKKMKNYWRQLLNAKLITQ | | | | |
| | | RKFDNLTKAERGGLSELDKAGFIKRQLVETRQITKHVAQILDS | | | | |
| | | RMNTKYDENDKLIREVKVITLKSKLVSDFRKDFQFYKVREIN | | | | |
| | | NYHHAHDAYLNAVVGTALIKKYPKLESEFVYGDYKVYDVR | | | | |
| | | KMIAKSEQEIGKATAKYFFYSNIMNFFKTEITLANGEIRKRPLI | | | | |
| | | ETNGETGEIVWDKGRDFATVRKVLSMPQVNIVKKTEVQTGG | | | | |
| | | FSKESIRPKRNSDKLIARKKDWDPKKYGGFVSPTVAYSVLVV | | | | |
| | | AKVEKGKSKKLKSVKELLGITIMERSSFEKNPIDFLEAKGYKE | | | | |
| | | VKKDLIIKLPKYSLFELENGRKRMLASARFLQKGNELALPSKY | | | | |
| | | VNFLYLASHYEKLKGSPEDNEQKQLFVEQHKHYLDEIIEQISE | | | | |
| | | FSKRVILADANLDKVLSAYNKHRDKPIREQAENIIHLFTLTNL | | | | |
| | | GAPRAFKYFDTTIDRKVYRSTKEVLDATLIHQSITGLYETRIDL | | | | |
| | | SQLGGD | | | | |
| Spy Cas9- | Streptococcus | MDKKYSIGLDIGTNSVGWAVITDEYKVPSKKFKVLGNTDRHS | 9,010 | N863A | H840A | D10A |
| SpRY | pyogenes | IKKNLIGALLFDSGETAERTRLKRTARRRYTRRKNRICYLQEIF | | | | |
| | | SNEMAKVDDSFFHRLEESFLVEEDKKHERHPIFGNIVDEVAY | | | | |
| | | HEKYPTIYHLRKKLVDSTDKADLRLIYLALAHMIKFRGHFLIE | | | | |
| | | GDLNPDNSDVDKLFIQLVQTYNQLFEENPINASGVDAKAILSA | | | | |
| | | RLSKSRRLENLIAQLPGEKKNGLFGNLIALSLGLTPNFKSNFDL | | | | |
| | | AEDAKLQLSKDTYDDDLDNLLAQIGDQYADLFLAAKNLSDA | | | | |
| | | ILLSDILRVNTEITKAPLSASMIKRYDEHHQDLTLLKALVRQQ | | | | |
| | | LPEKYKEIFFDQSKNGYAGYIDGGASQEEFYKFIKPILEKMDG | | | | |
| | | TEELLVKLNREDLLRKQRTFDNGSIPHQIHLGELHAILRRQED | | | | |
| | | FYPFLKDNREKIEKILTFRIPYYVGPLARGNSRFAWMTRKSEE | | | | |
| | | TITPWNFEEVVDKGASAQSFIERMTNFDKNLPNEKVLPKHSLL | | | | |
| | | YEYFTVYNELTKVKYVTEGMRKPAFLSGEQKKAIVDLLFKTN | | | | |
| | | RKVTVKQLKEDYFKKIECFDSVEISGVEDRFNASLGTYHDLL | | | | |
| | | KIIKDKDFLDNEENEDILEDIVLTLTLFEDREMIEERLKTYAHL | | | | |
| | | FDDKVMKQLKRRRYTGWGRLSRKLINGIRDKQSGKTILDFLK | | | | |
| | | SDGFANRNFMQLIHDDSLTFKEDIQKAQVSGQGDSLHEHIAN | | | | |
| | | LAGSPAIKKGILQTVKVVDELVKVMGRHKPENIVIEMARENQ | | | | |
| | | TTQKGQKNSRERMKRIEEGIKELGSQILKEHPVENTQLQNEKL | | | | |
| | | YLYYLQNGRDMYVDQELDINRLSDYDVDHIVPQSFLKDDSID | | | | |
| | | NKVLTRSDKNRGKSDNVPSEEVVKKMKNYWRQLLNAKLITQ | | | | |
| | | RKFDNLTKAERGGLSELDKAGFIKRQLVETRQITKHVAQILDS | | | | |
| | | RMNTKYDENDKLIREVKVITLKSKLVSDFRKDFQFYKVREIN | | | | |
| | | NYHHAHDAYLNAVVGTALIKKYPKLESEFVYGDYKVYDVR | | | | |
| | | KMIAKSEQEIGKATAKYFFYSNIMNFFKTEITLANGEIRKRPLI | | | | |
| | | ETNGETGEIVWDKGRDFATVRKVLSMPQVNIVKKTEVQTGG | | | | |
| | | FSKESIRPKRNSDKLIARKKDWDPKKYGGFLWPTVAYSVLVV | | | | |
| | | AKVEKGKSKKLKSVKELLGITIMERSSFEKNPIDFLEAKGYKE | | | | |
| | | VKKDLIIKLPKYSLFELENGRKRMLASAKQLQKGNELALPSK | | | | |
| | | YVNFLYLASHYEKLKGSPEDNEQKQLFVEQHKHYLDEIIEQIS | | | | |
| | | EFSKRVILADANLDKVLSAYNKHRDKPIREQAENIIHLFTLTR | | | | |
| | | LGAPRAFKYFDTTIDPKQYRSTKEVLDATLIHQSITGLYETRID | | | | |
| | | LSQLGGD | | | | |
| St1Cas9 | Streptococcus | MSDLVLGLDIGIGSVGVGILNKVTGEIIHKNSRIFPAAQAENNL | 9,011 | N622A | H599A | D9A |
| | thermophilus | VRRTNRQGRRLARRKKHRRVRLNRLFEESGLITDFTKISINLN | | | | |
| | | PYQLRVKGLTDELSNEELFIALKNMVKHRGISYLDDASDDGN | | | | |
| | | SSVGDYAQIVKENSKQLETKTPGQIQLERYQTYGQLRGDFTV | | | | |
| | | EKDGKKHRLINVFPTSAYRSEALRILQTQQEFNPQITDEFINRY | | | | |
| | | LEILTGKRKYYHGPGNEKSRTDYGRYRTSGETLDNIFGILIGK | | | | |
| | | CTFYPDEFRAAKASYTAQEFNLLNDLNNLTVPTETKKLSKEQ | | | | |
| | | KNQIINYVKNEKAMGPAKLFKYIAKLLSCDVADIKGYRIDKS | | | | |
| | | GKAEIHTFEAYRKMKTLETLDIEQMDRETLDKLAYVLTLNTE | | | | |
| | | REGIQEALEHEFADGSFSQKQVDELVQFRKANSSIFGKGWHN | | | | |
| | | FSVKLMMELIPELYETSEEQMTILTRLGKQKTTSSSNKTKYID | | | | |
| | | EKLLTEEIYNPVVAKSVRQAIKIVNAAIKEYGDFDNIVIEMAR | | | | |
| | | ETNEDDEKKAIQKIQKANKDEKDAAMLKAANQYNGKAELP | | | | |
| | | HSVFHGHKQLATKIRLWHQQGERCLYTGKTISIHDLINNSNQF | | | | |
| | | EVDHILPLSITFDDSLANKVLVYATANQEKGQRTPYQALDSM | | | | |
| | | DDAWSFRELKAFVRESKTLSNKKKEYLLTEEDISKFDVRKKFI | | | | |
| | | ERNLVDTRYASRVVLNALQEHFRAHKIDTKVSVVRGQFTSQL | | | | |
| | | RRHWGIEKTRDTYHHHAVDALIIAASSQLNLWKKQKNTLVS | | | | |
| | | YSEDQLLDIETGELISDDEYKESVFKAPYQHFVDTLKSKEFED | | | | |
| | | SILFSYQVDSKFNRKISDATIYATRQAKVGKDKADETYVLGKI | | | | |
| | | KDIYTQDGYDAFMKIYKKDKSKFLMYRHDPQTFEKVIEPILE | | | | |
| | | NYPNKQINEKGKEVPCNPFLKYKEEHGYIRKYSKKGNGPEIK | | | | |
| | | SLKYYDSKLGNHIDITPKDSNNKVVLQSVSPWRADVYFNKTT | | | | |
| | | GKYEILGLKYADLQFEKGTGTYKISQEKYNDIKKKEGVDSDS | | | | |
| | | EFKFTLYKNDLLLVKDTETKEQQLFRFLSRTMPKQKHYVELK | | | | |
| | | PYDKQKFEGGEALIKVLGNVANSGQCKKGLGKSNISIYKVRT | | | | |
| | | DVLGNQHIIKNEGDKPKLDF | | | | |
| BlatCas9 | Brevibacillus | MAYTMGIDVGIASCGWAIVDLERQRIIDIGVRTFEKAENPKNG | 9,012 | N607A | H584A | D8A |
| | laterosporus | EALAVPRREARSSRRRLRRKKHRIERLKHMFVRNGLAVDIQH | | | | |
| | | LEQTLRSQNEIDVWQLRVDGLDRMLTQKEWLRVLIHLAQRR | | | | |
| | | GFQSNRKTDGSSEDGQVLVNVTENDRLMEEKDYRTVAEMM | | | | |
| | | VKDEKFSDHKRNKNGNYHGVVSRSSLLVEIHTLFETQRQHHN | | | | |
| | | SLASKDFELEYVNIWSAQRPVATKDQIEKMIGTCTFLPKEKRA | | | | |
| | | PKASWHFQYFMLLQTINHIRITNVQGTRSLNKEEIEQVVNMA | | | | |
| | | LTKSKVSYHDTRKILDLSEEYQFVGLDYGKEDEKKKVESKET | | | | |
| | | IIKLDDYHKLNKIFNEVELAKGETWEADDYDTVAYALTFFKD | | | | |
| | | DEDIRDYLQNKYKDSKNRLVKNLANKEYTNELIGKVSTLSFR | | | | |
| | | KVGHLSLKALRKIIPFLEQGMTYDKACQAAGFDFQGISKKKR | | | | |
| | | SVVLPVIDQISNPVVNRALTQTRKVINALIKKYGSPETIHIETA | | | | |
| | | RELSKTFDERKNITKDYKENRDKNEHAKKHLSELGIINPTGLD | | | | |
| | | IVKYKLWCEQQGRCMYSNQPISFERLKESGYTEVDHIIPYSRS | | | | |
| | | MNDSYNNRVLVMTRENREKGNQTPFEYMGNDTQRWYEFEQ | | | | |
| | | RVTTNPQIKKEKRQNLLLKGFTNRRELEMLERNLNDTRYITK | | | | |
| | | YLSHFISTNLEFSPSDKKKKVVNTSGRITSHLRSRWGLEKNRG | | | | |
| | | QNDLHHAMDAIVIAVTSDSFIQQVTNYYKRKERRELNGDDKF | | | | |
| | | PLPWKFFREEVIARLSPNPKEQIEALPNHFYSEDELADLQPIFV | | | | |
| | | SRMPKRSITGEAHQAQFRRVVGKTKEGKNITAKKTALVDISY | | | | |
| | | DKNGDFNMYGRETDPATYEAIKERYLEFGGNVKKAFSTDLH | | | | |
| | | KPKKDGTKGPLIKSVRIMENKTLVHPVNKGKGVVYNSSIVRT | | | | |
| | | DVFQRKEKYYLLPVYVTDVTKGKLPNKVIVAKKGYHDWIEV | | | | |
| | | DDSFTFLFSLYPNDLIFIRQNPKKKISLKKRIESHSISDSKEVQEI | | | | |
| | | HAYYKGVDSSTAAIEFIIHDGSYYAKGVGVQNLDCFEKYQVD | | | | |
| | | ILGNYFKVKGEKRLELETSDSNHKGKDVNSIKSTSR | | | | |
| cCas9-v16 | Staphylococcus | MKRNYILGLDIGITSVGYGIIDYETRDVIDAGVRLFKEANVEN | 9,013 | N580A | H557A | D10A |
| | aureus | NEGRRSKRGARRLKRRRRHRIQRVKKLLFDYNLLTDHSELSG | | | | |
| | | INPYEARVKGLSQKLSEEEFSAALLHLAKRRGVHNVNEVEED | | | | |
| | | TGNELSTKEQISRNSKALEEKYVAELQLERLKKDGEVRGSINR | | | | |
| | | FKTSDYVKEAKQLLKVQKAYHQLDQSFIDTYIDLLETRRTYY | | | | |
| | | EGPGEGSPFGWKDIKEWYEMLMGHCTYFPEELRSVKYAYNA | | | | |
| | | DLYNALNDLNNLVITRDENEKLEYYEKFQIIENVFKQKKKPTL | | | | |
| | | KQIAKEILVNEEDIKGYRVTSTGKPEFTNLKVYHDIKDITARK | | | | |
| | | EIIENAELLDQIAKILTIYQSSEDIQEELTNLNSELTQEEIEQISN | | | | |
| | | LKGYTGTHNLSLKAINLILDELWHTNDNQIAIFNRLKLVPKKV | | | | |
| | | DLSQQKEIPTTLVDDFILSPVVKRSFIQSIKVINAIIKKYGLPNDI | | | | |
| | | IIELAREKNSKDAQKMINEMQKRNRQTNERIEEIIRTTGKENA | | | | |
| | | KYLIEKIKLHDMQEGKCLYSLEAIPLEDLLNNPFNYEVDHIIPR | | | | |
| | | SVSFDNSFNNKVLVKQEENSKKGNRTPFQYLSSSDSKISYETF | | | | |
| | | KKHILNLAKGKGRISKTKKEYLLEERDINRFSVQKDFINRNLV | | | | |
| | | DTRYATRGLMNLLRSYFRVNNLDVKVKSINGGFTSFLRRKW | | | | |
| | | KFKKERNKGYKHHAEDALIIANADFIFKEWKKLDKAKKVME | | | | |
| | | NQMFEEKQAESMPEIETEQEYKEIFITPHQIKHIKDFKDYKYSH | | | | |
| | | RVDKKPNRKLINDTLYSTRKDDKGNTLIVNNLNGLYDKDND | | | | |
| | | KLKKLINKSPEKLLMYHHDPQTYQKLKLIMEQYGDEKNPLY | | | | |
| | | KYYEETGNYLTKYSKKDNGPVIKKIKYYGNKLNAHLDITDD | | | | |
| | | YPNSRNKVVKLSLKPYRFDVYLDNGVYKFVTVKNLDVIKKE | | | | |
| | | NYYEVNSKCYEEAKKLKKISNQAEFIASFYKNDLIKINGELYR | | | | |
| | | VIGVNSDKNNLIEVNMIDITYREYLENMNDKRPPHIIKTIASKT | | | | |
| | | QSIKKYSTDILGNLYEVKSKKHPQIIKKG | | | | |
| cCas9-v17 | Staphylococcus | MKRNYILGLDIGITSVGYGIIDYETRDVIDAGVRLFKEANVEN | 9,014 | N580A | H557A | D10A |
| | aureus | NEGRRSKRGARRLKRRRRHRIQRVKKLLFDYNLLTDHSELSG | | | | |
| | | INPYEARVKGLSQKLSEEEFSAALLHLAKRRGVHNVNEVEED | | | | |
| | | TGNELSTKEQISRNSKALEEKYVAELQLERLKKDGEVRGSINR | | | | |
| | | FKTSDYVKEAKQLLKVQKAYHQLDQSFIDTYIDLLETRRTYY | | | | |
| | | EGPGEGSPFGWKDIKEWYEMLMGHCTYFPEELRSVKYAYNA | | | | |
| | | DLYNALNDLNNLVITRDENEKLEYYEKFQIIENVFKQKKKPTL | | | | |
| | | KQIAKEILVNEEDIKGYRVTSTGKPEFTNLKVYHDIKDITARK | | | | |
| | | EIIENAELLDQIAKILTIYQSSEDIQEELTNLNSELTQEEIEQISN | | | | |
| | | LKGYTGTHNLSLKAINLILDELWHTNDNQIAIFNRLKLVPKKV | | | | |
| | | DLSQQKEIPTTLVDDFILSPVVKRSFIQSIKVINAIIKKYGLPNDI | | | | |
| | | IIELAREKNSKDAQKMINEMQKRNRQTNERIEEIIRTTGKENA | | | | |
| | | KYLIEKIKLHDMQEGKCLYSLEAIPLEDLLNNPFNYEVDHIIPR | | | | |
| | | SVSFDNSFNNKVLVKQEENSKKGNRTPFQYLSSSDSKISYETF | | | | |
| | | KKHILNLAKGKGRISKTKKEYLLEERDINRFSVQKDFINRNLV | | | | |
| | | DTRYATRGLMNLLRSYFRVNNLDVKVKSINGGFTSFLRRKW | | | | |
| | | KFKKERNKGYKHHAEDALIIANADFIFKEWKKLDKAKKVME | | | | |
| | | NQMFEEKQAESMPEIETEQEYKEIFITPHQIKHIKDFKDYKYSH | | | | |
| | | RVDKKPNRKLINDTLYSTRKDDKGNTLIVNNLNGLYDKDND | | | | |
| | | KLKKLINKSPEKLLMYHHDPQTYQKLKLIMEQYGDEKNPLY | | | | |
| | | KYYEETGNYLTKYSKKDNGPVIKKIKYYGNKLNAHLDITDD | | | | |
| | | YPNSRNKVVKLSLKPYRFDVYLDNGVYKFVTVKNLDVIKKE | | | | |
| | | NYYEVNSKCYEEAKKLKKISNQAEFIASFYKNDLIKINGELYR | | | | |
| | | VIGVNNSTRNIVELNMIDITYREYLENMNDKRPPHIIKTIASKT | | | | |
| | | QSIKKYSTDILGNLYEVKSKKHPQIIKKG | | | | |
| cCas9-v21 | Staphylococcus | MKRNYILGLDIGITSVGYGIIDYETRDVIDAGVRLFKEANVEN | 9,015 | N580A | H557A | D10A |
| | aureus | NEGRRSKRGARRLKRRRRHRIQRVKKLLFDYNLLTDHSELSG | | | | |
| | | INPYEARVKGLSQKLSEEEFSAALLHLAKRRGVHNVNEVEED | | | | |
| | | TGNELSTKEQISRNSKALEEKYVAELQLERLKKDGEVRGSINR | | | | |
| | | FKTSDYVKEAKQLLKVQKAYHQLDQSFIDTYIDLLETRRTYY | | | | |
| | | EGPGEGSPFGWKDIKEWYEMLMGHCTYFPEELRSVKYAYNA | | | | |
| | | DLYNALNDLNNLVITRDENEKLEYYEKFQIIENVFKQKKKPTL | | | | |
| | | KQIAKEILVNEEDIKGYRVTSTGKPEFTNLKVYHDIKDITARK | | | | |
| | | EIIENAELLDQIAKILTIYQSSEDIQEELTNLNSELTQEEIEQISN | | | | |
| | | LKGYTGTHNLSLKAINLILDELWHTNDNQIAIFNRLKLVPKKV | | | | |
| | | DLSQQKEIPTTLVDDFILSPVVKRSFIQSIKVINAIIKKYGLPNDI | | | | |
| | | IIELAREKNSKDAQKMINEMQKRNRQTNERIEEIIRTTGKENA | | | | |
| | | KYLIEKIKLHDMQEGKCLYSLEAIPLEDLLNNPFNYEVDHIIPR | | | | |
| | | SVSFDNSFNNKVLVKQEENSKKGNRTPFQYLSSSDSKISYETF | | | | |
| | | KKHILNLAKGKGRISKTKKEYLLEERDINRFSVQKDFINRNLV | | | | |
| | | DTRYATRGLMNLLRSYFRVNNLDVKVKSINGGFTSFLRRKW | | | | |
| | | KFKKERNKGYKHHAEDALIIANADFIFKEWKKLDKAKKVME | | | | |
| | | NQMFEEKQAESMPEIETEQEYKEIFITPHQIKHIKDFKDYKYSH | | | | |
| | | RVDKKPNRKLINDTLYSTRKDDKGNTLIVNNLNGLYDKDND | | | | |
| | | KLKKLINKSPEKLLMYHHDPQTYQKLKLIMEQYGDEKNPLY | | | | |
| | | KYYEETGNYLTKYSKKDNGPVIKKIKYYGNKLNAHLDITDD | | | | |
| | | YPNSRNKVVKLSLKPYRFDVYLDNGVYKFVTVKNLDVIKKE | | | | |
| | | NYYEVNSKCYEEAKKLKKISNQAEFIASFYKNDLIKINGELYR | | | | |
| | | VIGVNSDDRNIIELNMIDITYREYLENMNDKRPPHIIKTIASKT | | | | |
| | | QSIKKYSTDILGNLYEVKSKKHPQIIKKG | | | | |
| cCas9-v42 | Staphylococcus | MKRNYILGLDIGITSVGYGIIDYETRDVIDAGVRLFKEANVEN | 9,016 | N580A | H557A | D10A |
| | aureus | NEGRRSKRGARRLKRRRRHRIQRVKKLLFDYNLLTDHSELSG | | | | |
| | | INPYEARVKGLSQKLSEEEFSAALLHLAKRRGVHNVNEVEED | | | | |
| | | TGNELSTKEQISRNSKALEEKYVAELQLERLKKDGEVRGSINR | | | | |
| | | FKTSDYVKEAKQLLKVQKAYHQLDQSFIDTYIDLLETRRTYY | | | | |
| | | EGPGEGSPFGWKDIKEWYEMLMGHCTYFPEELRSVKYAYNA | | | | |
| | | DLYNALNDLNNLVITRDENEKLEYYEKFQIIENVFKQKKKPTL | | | | |
| | | KQIAKEILVNEEDIKGYRVTSTGKPEFTNLKVYHDIKDITARK | | | | |
| | | EIIENAELLDQIAKILTIYQSSEDIQEELTNLNSELTQEEIEQISN | | | | |
| | | LKGYTGTHNLSLKAINLILDELWHTNDNQIAIFNRLKLVPKKV | | | | |
| | | DLSQQKEIPTTLVDDFILSPVVKRSFIQSIKVINAIIKKYGLPNDI | | | | |
| | | IIELAREKNSKDAQKMINEMQKRNRQTNERIEEIIRTTGKENA | | | | |
| | | KYLIEKIKLHDMQEGKCLYSLEAIPLEDLLNNPFNYEVDHIIPR | | | | |
| | | SVSFDNSFNNKVLVKQEENSKKGNRTPFQYLSSSDSKISYETF | | | | |
| | | KKHILNLAKGKGRISKTKKEYLLEERDINRFSVQKDFINRNLV | | | | |
| | | DTRYATRGLMNLLRSYFRVNNLDVKVKSINGGFTSFLRRKW | | | | |
| | | KFKKERNKGYKHHAEDALIIANADFIFKEWKKLDKAKKVME | | | | |
| | | NQMFEEKQAESMPEIETEQEYKEIFITPHQIKHIKDFKDYKYSH | | | | |
| | | RVDKKPNRKLINDTLYSTRKDDKGNTLIVNNLNGLYDKDND | | | | |
| | | KLKKLINKSPEKLLMYHHDPQTYQKLKLIMEQYGDEKNPLY | | | | |
| | | KYYEETGNYLTKYSKKDNGPVIKKIKYYGNKLNAHLDITDD | | | | |
| | | YPNSRNKVVKLSLKPYRFDVYLDNGVYKFVTVKNLDVIKKE | | | | |
| | | NYYEVNSKCYEEAKKLKKISNQAEFIASFYKNDLIKINGELYR | | | | |
| | | VIGVNNNRLNKIELNMIDITYREYLENMNDKRPPHIIKTIASKT | | | | |
| | | QSIKKYSTDILGNLYEVKSKKHPQIIKKG | | | | |
| CdiCas9 | Corynebacterium | MKYHVGIDVGTFSVGLAAIEVDDAGMPIKTLSLVSHIHDSGL | 9,017 | N597A | H573A | D8A |
| | diphtheriae | DPDEIKSAVTRLASSGIARRTRRLYRRKRRRLQQLDKFIQRQG | | | | |
| | | WPVIELEDYSDPLYPWKVRAELAASYIADEKERGEKLSVALR | | | | |
| | | HIARHRGWRNPYAKVSSLYLPDGPSDAFKAIREEIKRASGQPV | | | | |
| | | PETATVGQMVTLCELGTLKLRGEGGVLSARLQQSDYAREIQE | | | | |
| | | ICRMQEIGQELYRKIIDVVFAAESPKGSASSRVGKDPLQPGKN | | | | |
| | | RALKASDAFQRYRIAALIGNLRVRVDGEKRILSVEEKNLVFD | | | | |
| | | HLVNLTPKKEPEWVTIAEILGIDRGQLIGTATMTDDGERAGA | | | | |
| | | RPPTHDTNRSIVNSRIAPLVDWWKTASALEQHAMVKALSNA | | | | |
| | | EVDDFDSPEGAKVQAFFADLDDDVHAKLDSLHLPVGRAAYS | | | | |
| | | EDTLVRLTRRMLSDGVDLYTARLQEFGIEPSWTPPTPRIGEPV | | | | |
| | | GNPAVDRVLKTVSRWLESATKTWGAPERVIIEHVREGFVTEK | | | | |
| | | RAREMDGDMRRRAARNAKLFQEMQEKLNVQGKPSRADLW | | | | |
| | | RYQSVQRQNCQCAYCGSPITFSNSEMDHIVPRAGQGSTNTRE | | | | |
| | | NLVAVCHRCNQSKGNTPFAIWAKNTSIEGVSVKEAVERTRH | | | | |
| | | WVTDTGMRSTDFKKFTKAVVERFQRATMDEEIDARSMESVA | | | | |
| | | WMANELRSRVAQHFASHGTTVRVYRGSLTAEARRASGISGK | | | | |
| | | LKFFDGVGKSRLDRRHHAIDAAVIAFTSDYVAETLAVRSNLK | | | | |
| | | QSQAHRQEAPQWREFTGKDAEHRAAWRVWCQKMEKLSALL | | | | |
| | | TEDLRDDRVVVMSNVRLRLGNGSAHKETIGKLSKVKLSSQLS | | | | |
| | | VSDIDKASSEALWCALTREPGFDPKEGLPANPERHIRVNGTH | | | | |
| | | VYAGDNIGLFPVSAGSIALRGGYAELGSSFHHARVYKITSGKK | | | | |
| | | PAFAMLRVYTIDLLPYRNQDLFSVELKPQTMSMRQAEKKLR | | | | |
| | | DALATGNAEYLGWLVVDDELVVDTSKIATDQVKAVEAELGT | | | | |
| | | IRRWRVDGFFSPSKLRLRPLQMSKEGIKKESAPELSKIIDRPGW | | | | |
| | | LPAVNKLFSDGNVTVVRRDSLGRVRLESTAHLPVTWKVQ | | | | |
| CjeCas9 | Campylobacter | MARILAFDIGISSIGWAFSENDELKDCGVRIFTKVENPKTGESL | 9,018 | N582A | H559A | D8A |
| | jejuni | ALPRRLARSARKRLARRKARLNHLKHLIANEFKLNYEDYQSF | | | | |
| | | DESLAKAYKGSLISPYELRFRALNELLSKQDFARVILHIAKRR | | | | |
| | | GYDDIKNSDDKEKGAILKAIKQNEEKLANYQSVGEYLYKEYF | | | | |
| | | QKFKENSKEFTNVRNKKESYERCIAQSFLKDELKLIFKKQREF | | | | |
| | | GFSFSKKFEEEVLSVAFYKRALKDFSHLVGNCSFFTDEKRAPK | | | | |
| | | NSPLAFMFVALTRIINLLNNLKNTEGILYTKDDLNALLNEVLK | | | | |
| | | NGTLTYKQTKKLLGLSDDYEFKGEKGTYFIEFKKYKEFIKAL | | | | |
| | | GEHNLSQDDLNEIAKDITLIKDEIKLKKALAKYDLNQNQIDSL | | | | |
| | | SKLEFKDHLNISFKALKLVTPLMLEGKKYDEACNELNLKVAI | | | | |
| | | NEDKKDFLPAFNETYYKDEVTNPVVLRAIKEYRKVLNALLK | | | | |
| | | KYGKVHKINIELAREVGKNHSQRAKIEKEQNENYKAKKDAE | | | | |
| | | LECEKLGLKINSKNILKLRLFKEQKEFCAYSGEKIKISDLQDEK | | | | |
| | | MLEIDHIYPYSRSFDDSYMNKVLVFTKQNQEKLNQTPFEAFG | | | | |
| | | NDSAKWQKIEVLAKNLPTKKQKRILDKNYKDKEQKNFKDRN | | | | |
| | | LNDTRYIARLVLNYTKDYLDFLPLSDDENTKLNDTQKGSKVH | | | | |
| | | VEAKSGMLTSALRHTWGFSAKDRNNHLHHAIDAVIIAYANN | | | | |
| | | SIVKAFSDFKKEQESNSAELYAKKISELDYKNKRKFFEPFSGF | | | | |
| | | RQKVLDKIDEIFVSKPERKKPSGALHEETFRKEEEFYQSYGGK | | | | |
| | | EGVLKALELGKIRKVNGKIVKNGDMFRVDIFKHKKTNKFYA | | | | |
| | | VPIYTMDFALKVLPNKAVARSKKGEIKDWILMDENYEFCFSL | | | | |
| | | YKDSLILIQTKDMQEPEFVYYNAFTSSTVSLIVSKHDNKFETLS | | | | |
| | | KNQKILFKNANEKEVIAKSIGIQNLKVFEKYIVSALGEVTKAE | | | | |
| | | FRQREDFKK | | | | |
| GeoCas9 | Geobacillus | MRYKIGLDIGITSVGWAVMNLDIPRIEDLGVRIFDRAENPQTG | 9,019 | N605A | H582A | D8A |
| | stearothermophilus | ESLALPRRLARSARRRLRRRKHRLERIRRLVIREGILTKEELDK | | | | |
| | | LFEEKHEIDVWQLRVEALDRKLNNDELARVLLHLAKRRGFK | | | | |
| | | SNRKSERSNKENSTMLKHIEENRAILSSYRTVGEMIVKDPKFA | | | | |
| | | LHKRNKGENYTNTIARDDLEREIRLIFSKQREFGNMSCTEEFE | | | | |
| | | NEYITIWASQRPVASKDDIEKKVGFCTFEPKEKRAPKATYTFQ | | | | |
| | | SFIAWEHINKLRLISPSGARGLTDEERRLLYEQAFQKNKITYH | | | | |
| | | DIRTLLHLPDDTYFKGIVYDRGESRKQNENIRFLELDAYHQIR | | | | |
| | | KAVDKVYGKGKSSSFLPIDFDTFGYALTLFKDDADIHSYLRN | | | | |
| | | EYEQNGKRMPNLANKVYDNELIEELLNLSFTKFGHLSLKALR | | | | |
| | | SILPYMEQGEVYSSACERAGYTFTGPKKKQKTMLLPNIPPIAN | | | | |
| | | PVVMRALTQARKVVNAIIKKYGSPVSIHIELARDLSQTFDERR | | | | |
| | | KTKKEQDENRKKNETAIRQLMEYGLTLNPTGHDIVKFKLWSE | | | | |
| | | QNGRCAYSLQPIEIERLLEPGYVEVDHVIPYSRSLDDSYTNKV | | | | |
| | | LVLTRENREKGNRIPAEYLGVGTERWQQFETFVLTNKQFSKK | | | | |
| | | KRDRLLRLHYDENEETEFKNRNLNDTRYISRFFANFIREHLKF | | | | |
| | | AESDDKQKVYTVNGRVTAHLRSRWEFNKNREESDLHHAVD | | | | |
| | | AVIVACTTPSDIAKVTAFYQRREQNKELAKKTEPHFPQPWPH | | | | |
| | | FADELRARLSKHPKESIKALNLGNYDDQKLESLQPVFVSRMP | | | | |
| | | KRSVTGAAHQETLRRYVGIDERSGKIQTVVKTKLSEIKLDASG | | | | |
| | | HFPMYGKESDPRTYEAIRQRLLEHNNDPKKAFQEPLYKPKKN | | | | |
| | | GEPGPVIRTVKIIDTKNQVIPLNDGKTVAYNSNIVRVDVFEKD | | | | |
| | | GKYYCVPVYTMDIMKGILPNKAIEPNKPYSEWKEMTEDYTFR | | | | |
| | | FSLYPNDLIRIELPREKTVKTAAGEEINVKDVFVYYKTIDSAN | | | | |
| | | GGLELISHDHRFSLRGVGSRTLKRFEKYQVDVLGNIYKVRGE | | | | |
| | | KRVGLASSAHSKPGKTIRPLQSTRD | | | | |
| iSpyMacCas9 | Streptococcus | MDKKYSIGLDIGTNSVGWAVITDEYKVPSKKFKVLGNTDRHS | 9,020 | N863A | H840A | D10A |
| | spp. | IKKNLIGALLFDSGETAEATRLKRTARRRYTRRKNRICYLQEIF | | | | |
| | | SNEMAKVDDSFFHRLEESFLVEEDKKHERHPIFGNIVDEVAY | | | | |
| | | HEKYPTIYHLRKKLVDSTDKADLRLIYLALAHMIKFRGHFLIE | | | | |
| | | GDLNPDNSDVDKLFIQLVQTYNQLFEENPINASGVDAKAILSA | | | | |
| | | RLSKSRKLENLIAQLPGEKKNGLFGNLIALSLGLTPNFKSNFD | | | | |
| | | LAEDAKLQLSKDTYDDDLDNLLAQIGDQYADLFLAAKNLSD | | | | |
| | | AILLSDILRVNTEITKAPLSASMIKRYDEHHQDLTLLKALVRQ | | | | |
| | | QLPEKYKEIFFDQSKNGYAGYIDGGASQEEFYKFIKPILEKMD | | | | |
| | | GTEELLVKLKREDLLRKQRTFDNGSIPHQIHLGELHAILRRQE | | | | |
| | | DFYPFLKDNREKIEKILTFRIPYYVGPLARGNSRFAWMIRKSE | | | | |
| | | ETITPWNFEEVVDKGASAQSFIERMTNFDKNLPNEKVLPKHSL | | | | |
| | | LYEYFTVYNELTKVKYVTEGMRKPAFLSGEQKKAIVDLLFKT | | | | |
| | | NRKVTVKQLKEDYFKKIECFDSVEISGVEDRFNASLGTYHDL | | | | |
| | | LKIIKDKDFLDNEENEDILEDIVLTLTLFEDREMIEERLKTYAH | | | | |
| | | LFDDKVMKQLKRRRYTGWGRLSRKLINGIRDKQSGKTILDFL | | | | |
| | | KSDGFANRNFMQLIHDDSLTFKEDIQKAQVSGQGDSLHEHIA | | | | |
| | | NLAGSPAIKKGILQTVKVVDELVKVMGRHKPENIVIEMAREN | | | | |
| | | QTTQKGQKNSRERMKRIEEGIKELGSQILKEHPVENTQLQNE | | | | |
| | | KLYLYYLQNGRDMYVDQELDINRLSDYDVDHIVPQSFLKDD | | | | |
| | | SIDNKVLTRSDKNRGKSDNVPSEEVVKKMKNYWRQLLNAKL | | | | |
| | | ITQRKFDNLTKAERGGLSELDKAGFIKRQLVETRQITKHVAQI | | | | |
| | | LDSRMNTKYDENDKLIREVKVITLKSKLVSDFRKDFQFYKVR | | | | |
| | | EINNYHHAHDAYLNAVVGTALIKKYPKLESEFVYGDYKVYD | | | | |
| | | VRKMIAKSEQEIGKATAKYFFYSNIMNFFKTEITLANGEIRKR | | | | |
| | | PLIETNGETGEIVWDKGRDFATVRKVLSMPQVNIVKKTEIQTV | | | | |
| | | GQNGGLFDDNPKSPLEVTPSKLVPLKKELNPKKYGGYQKPTT | | | | |
| | | AYPVLLITDTKQLIPISVMNKKQFEQNPVKFLRDRGYQQVGK | | | | |
| | | NDFIKLPKYTLVDIGDGIKRLWASSKEIHKGNQLVVSKKSQIL | | | | |
| | | LYHAHHLDSDLSNDYLQNHNQQFDVLFNEIISFSKKCKLGKE | | | | |
| | | HIQKIENVYSNKKNSASIEELAESFIKLLGFTQLGATSPFNFLG | | | | |
| | | VKLNQKQYKGKKDYILPCTEGTLIRQSITGLYETRVDLSKIGE | | | | |
| | | DSGGSGGSKRTADGSEFES | | | | |
| NmeCas9 | Neisseria | MAAFKPNSINYILGLDIGIASVGWAMVEIDEEENPIRLIDLGVR | 9,021 | N611A | H588A | D16A |
| | meningitidis | VFERAEVPKTGDSLAMARRLARSVRRLTRRRAHRLLRTRRLL | | | | |
| | | KREGVLQAANFDENGLIKSLPNTPWQLRAAALDRKLTPLEWS | | | | |
| | | AVLLHLIKHRGYLSQRKNEGETADKELGALLKGVAGNAHAL | | | | |
| | | QTGDFRTPAELALNKFEKESGHIRNQRSDYSHTFSRKDLQAEL | | | | |
| | | ILLFEKQKEFGNPHVSGGLKEGIETLLMTQRPALSGDAVQKM | | | | |
| | | LGHCTFEPAEPKAAKNTYTAERFIWLTKLNNLRILEQGSERPL | | | | |
| | | TDTERATLMDEPYRKSKLTYAQARKLLGLEDTAFFKGLRYG | | | | |
| | | KDNAEASTLMEMKAYHAISRALEKEGLKDKKSPLNLSPELQD | | | | |
| | | EIGTAFSLFKTDEDITGRLKDRIQPEILEALLKHISFDKFVQISL | | | | |
| | | KALRRIVPLMEQGKRYDEACAEIYGDHYGKKNTEEKIYLPPIP | | | | |
| | | ADEIRNPVVLRALSQARKVINGVVRRYGSPARIHIETAREVGK | | | | |
| | | SFKDRKEIEKRQEENRKDREKAAAKFREYFPNFVGEPKSKDIL | | | | |
| | | KLRLYEQQHGKCLYSGKEINLGRLNEKGYVEIDHALPFSRTW | | | | |
| | | DDSFNNKVLVLGSENQNKGNQTPYEYFNGKDNSREWQEFKA | | | | |
| | | RVETSRFPRSKKQRILLQKFDEDGFKERNLNDTRYVNRFLCQF | | | | |
| | | VADRMRLTGKGKKRVFASNGQITNLLRGFWGLRKVRAEND | | | | |
| | | RHHALDAVVVACSTVAMQQKITRFVRYKEMNAFDGKTIDKE | | | | |
| | | TGEVLHQKTHFPQPWEFFAQEVMIRVFGKPDGKPEFEEADTL | | | | |
| | | EKLRTLLAEKLSSRPEAVHEYVTPLFVSRAPNRKMSGQGHME | | | | |
| | | TVKSAKRLDEGVSVLRVPLTQLKLKDLEKMVNREREPKLYE | | | | |
| | | ALKARLEAHKDDPAKAFAEPFYKYDKAGNRTQQVKAVRVE | | | | |
| | | QVQKTGVWVRNHNGIADNATMVRVDVFEKGDKYYLVPIYS | | | | |
| | | WQVAKGILPDRAVVQGKDEEDWQLIDDSFNFKFSLHPNDLV | | | | |
| | | EVITKKARMFGYFASCHRGTGNINIRIHDLDHKIGKNGILEGIG | | | | |
| | | VKTALSFQKYQIDELGKEIRPCRLKKRPPVR | | | | |
| ScaCas9 | Streptococcus | MEKKYSIGLDIGTNSVGWAVITDDYKVPSKKFKVLGNTNRKS | 9,022 | N872A | H849A | D10A |
| | canis | IKKNLMGALLFDSGETAEATRLKRTARRRYTRRKNRIRYLQEI | | | | |
| | | FANEMAKLDDSFFQRLEESFLVEEDKKNERHPIFGNLADEVA | | | | |
| | | YHRNYPTIYHLRKKLADSPEKADLRLIYLALAHIIKFRGHFLIE | | | | |
| | | GKLNAENSDVAKLFYQLIQTYNQLFEESPLDEIEVDAKGILSA | | | | |
| | | RLSKSKRLEKLIAVFPNEKKNGLFGNIIALALGLTPNFKSNFDL | | | | |
| | | TEDAKLQLSKDTYDDDLDELLGQIGDQYADLFSAAKNLSDAI | | | | |
| | | LLSDILRSNSEVTKAPLSASMVKRYDEHHQDLALLKTLVRQQ | | | | |
| | | FPEKYAEIFKDDTKNGYAGYVGIGIKHRKRTTKLATQEEFYK | | | | |
| | | FIKPILEKMDGAEELLAKLNRDDLLRKQRTFDNGSIPHQIHLK | | | | |
| | | ELHAILRRQEEFYPFLKENREKIEKILTFRIPYYVGPLARGNSR | | | | |
| | | FAWLTRKSEEAITPWNFEEVVDKGASAQSFIERMTNFDEQLP | | | | |
| | | NKKVLPKHSLLYEYFTVYNELTKVKYVTERMRKPEFLSGEQ | | | | |
| | | KKAIVDLLFKTNRKVTVKQLKEDYFKKIECFDSVEIIGVEDRF | | | | |
| | | NASLGTYHDLLKIIKDKDFLDNEENEDILEDIVLTLTLFEDRE | | | | |
| | | MIEERLKTYAHLFDDKVMKQLKRRHYTGWGRLSRKMINGIR | | | | |
| | | DKQSGKTILDFLKSDGFSNRNFMQLIHDDSLTFKEEIEKAQVS | | | | |
| | | GQGDSLHEQIADLAGSPAIKKGILQTVKIVDELVKVMGHKPE | | | | |
| | | NIVIEMARENQTTTKGLQQSRERKKRIEEGIKELESQILKENPV | | | | |
| | | ENTQLQNEKLYLYYLQNGRDMYVDQELDINRLSDYDVDHIV | | | | |
| | | PQSFIKDDSIDNKVLTRSVENRGKSDNVPSEEVVKKMKNYWR | | | | |
| | | QLLNAKLITQRKFDNLTKAERGGLSEADKAGFIKRQLVETRQI | | | | |
| | | TKHVARILDSRMNTKRDKNDKPIREVKVITLKSKLVSDFRKD | | | | |
| | | FQLYKVRDINNYHHAHDAYLNAVVGTALIKKYPKLESEFVY | | | | |
| | | GDYKVYDVRKMIAKSEQEIGKATAKRFFYSNIMNFFKTEVKL | | | | |
| | | ANGEIRKRPLIETNGETGEVVWNKEKDFATVRKVLAMPQVNI | | | | |
| | | VKKTEVQTGGFSKESILSKRESAKLIPRKKGWDTRKYGGFGS | | | | |
| | | PTVAYSILVVAKVEKGKAKKLKSVKVLVGITIMEKGSYEKDP | | | | |
| | | IGFLEAKGYKDIKKELIFKLPKYSLFELENGRRRMLASATELQ | | | | |
| | | KANELVLPQHLVRLLYYTQNISATTGSNNLGYIEQHREEFKEI | | | | |
| | | FEKIIDFSEKYILKNKVNSNLKSSFDEQFAVSDSILLSNSFVSLL | | | | |
| | | KYTSFGASGGFTFLDLDVKQGRLRYQTVTEVLDATLIYQSITG | | | | |
| | | LYETRTDLSQLGGD | | | | |
| ScaCas9- | Streptococcus | MEKKYSIGLDIGTNSVGWAVITDDYKVPSKKFKVLGNTNRKS | 9,023 | N872A | H849A | D10A |
| HiFi-Sc++ | canis | IKKNLMGALLFDSGETAEATRLKRTARRRYTRRKNRIRYLQEI | | | | |
| | | FANEMAKLDDSFFQRLEESFLVEEDKKNERHPIFGNLADEVA | | | | |
| | | YHRNYPTIYHLRKKLADSPEKADLRLIYLALAHIIKFRGHFLIE | | | | |
| | | GKLNAENSDVAKLFYQLIQTYNQLFEESPLDEIEVDAKGILSA | | | | |
| | | RLSKSKRLEKLIAVFPNEKKNGLFGNIIALALGLTPNFKSNFDL | | | | |
| | | TEDAKLQLSKDTYDDDLDELLGQIGDQYADLFSAAKNLSDAI | | | | |
| | | LLSDILRSNSEVTKAPLSASMVKRYDEHHQDLALLKTLVRQQ | | | | |
| | | FPEKYAEIFKDDTKNGYAGYVGADKKLRKRSGKLATEEEFY | | | | |
| | | KFIKPILEKMDGAEELLAKLNRDDLLRKQRTFDNGSIPHQIHL | | | | |
| | | KELHAILRRQEEFYPFLKENREKIEKILTFRIPYYVGPLARGNS | | | | |
| | | RFAWLTRKSEEAITPWNFEEVVDKGASAQSFIERMTNFDEQL | | | | |
| | | PNKKVLPKHSLLYEYFTVYNELTKVKYVTERMRKPEFLSGEQ | | | | |
| | | KKAIVDLLFKTNRKVTVKQLKEDYFKKIECFDSVEIIGVEDRF | | | | |
| | | NASLGTYHDLLKIIKDKDFLDNEENEDILEDIVLTLTLFEDRE | | | | |
| | | MIEERLKTYAHLFDDKVMKQLKRRHYTGWGRLSRKMINGIR | | | | |
| | | DKQSGKTILDFLKSDGFSNANFMQLIHDDSLTFKEEIEKAQVS | | | | |
| | | GQGDSLHEQIADLAGSPAIKKGILQTVKIVDELVKVMGHKPE | | | | |
| | | NIVIEMARENQTTTKGLQQSRERKKRIEEGIKELESQILKENPV | | | | |
| | | ENTQLQNEKLYLYYLQNGRDMYVDQELDINRLSDYDVDHIV | | | | |
| | | PQSFIKDDSIDNKVLTRSVENRGKSDNVPSEEVVKKMKNYWR | | | | |
| | | QLLNAKLITQRKFDNLTKAERGGLSEADKAGFIKRQLVETRQI | | | | |
| | | TKHVARILDSRMNTKRDKNDKPIREVKVITLKSKLVSDFRKD | | | | |
| | | FQLYKVRDINNYHHAHDAYLNAVVGTALIKKYPKLESEFVY | | | | |
| | | GDYKVYDVRKMIAKSEQEIGKATAKRFFYSNIMNFFKTEVKL | | | | |
| | | ANGEIRKRPLIETNGETGEVVWNKEKDFATVRKVLAMPQVNI | | | | |
| | | VKKTEVQTGGFSKESILSKRESAKLIPRKKGWDTRKYGGFGS | | | | |
| | | PTVAYSILVVAKVEKGKAKKLKSVKVLVGITIMEKGSYEKDP | | | | |
| | | IGFLEAKGYKDIKKELIFKLPKYSLFELENGRRRMLASAKELQ | | | | |
| | | KANELVLPQHLVRLLYYTQNISATTGSNNLGYIEQHREEFKEI | | | | |
| | | FEKIIDFSEKYILKNKVNSNLKSSFDEQFAVSDSILLSNSFVSLL | | | | |
| | | KYTSFGASGGFTFLDLDVKQGRLRYQTVTEVLDATLIYQSITG | | | | |
| | | LYETRTDLSQLGGD | | | | |
| SpyCas9- | Streptococcus | MDKKYSIGLDIGTNSVGWAVITDEYKVPSKKFKVLGNTDRHS | 9,024 | N863A | H840A | D10A |
| 3var- | pyogenes | IKKNLIGALLFDSGETAEATRLKRTARRRYTRRKNRICYLQEIF | | | | |
| NRRH | | SNEMAKVDDSFFHRLEESFLVEEDKKHERHPIFGNIVDEVAY | | | | |
| | | HEKYPTIYHLRKKLVDSTDKADLRLIYLALAHMIKFRGHFLIE | | | | |
| | | GDLNPDNSDVDKLFIQLVQTYNQLFEENPINASGVDAKAILSA | | | | |
| | | RLSKSRRLENLIAQLPGEKKNGLFGNLIALSLGLTPNFKSNFDL | | | | |
| | | AEDAKLQLSKDTYDDDLDNLLAQIGDQYADLFLAAKNLSDA | | | | |
| | | ILLSDILRVNTEITKAPLSASMVKRYDEHHQDLTLLKALVRQQ | | | | |
| | | LPEKYKEIFFDQSKNGYAGYIDGGASQEEFYKFIKPILEKMDG | | | | |
| | | TEELLVKLNREDLLRKQRTFDNGIIPHQIHLGELHAILRRQGDF | | | | |
| | | YPFLKDNREKIEKILTFRIPYYVGPLARGNSRFAWMTRKSEETI | | | | |
| | | TPWNFEEVVDKGASAQSFIERMTNFDKNLPNEKVLPKHSLLY | | | | |
| | | EYFTVYNELTKVKYVTEGMRKPAFLSGEQKKAIVDLLFKTNR | | | | |
| | | KVTVKQLKEDYFKKIECFDSVEISGVEDRFNASLGTYHDLLKI | | | | |
| | | IKDKDFLDNEENEDILEDIVLTLTLFEDREMIEERLKTYAHLFD | | | | |
| | | DKVMKQLKRLRYTGWGRLSRKLINGIRDKQSGKTILDFLKSD | | | | |
| | | GFANRNFMQLIHDDSLTFKEDIQKAQVSGQGDSLHEHIANLA | | | | |
| | | GSPAIKKGILQTVKVVDELVKVMGGHKPENIVIEMARENQTT | | | | |
| | | QKGQKNSRERMKRIEEGIKELGSQILKEHPVENTQLQNEKLY | | | | |
| | | LYYLQNGRDMYVDQELDINRLSDYDVDHIVPQSFLKDDSIDN | | | | |
| | | KVLTRSDKNRGKSDNVPSEEVVKKMKNYWRQLLNAKLITQR | | | | |
| | | KFDNLTKAERGGLSELDKAGFIKRQLVETRQITKHVAQILDSR | | | | |
| | | MNTKYDENDKLIREVKVITLKSKLVSDFRKDFQFYKVREINN | | | | |
| | | YHHAHDAYLNAVVGTALIKKYPKLESEFVYGDYKVYDVRK | | | | |
| | | MIAKSEQEIGKATAKYFFYSNIMNFFKTEITLANGEIRKRPLIE | | | | |
| | | TNGETGEIVWDKGRDFATVRKVLSMPQVNIVKKTEVQTGGF | | | | |
| | | SKESILPKGNSDKLIARKKDWDPKKYGGFNSPTAAYSVLVVA | | | | |
| | | KVEKGKSKKLKSVKELLGITIMERSSFEKNPIGFLEAKGYKEV | | | | |
| | | KKDLIIKLPKYSLFELENGRKRMLASAGVLHKGNELALPSKY | | | | |
| | | VNFLYLASHYEKLKGSPEDNEQKQLFVEQHKHYLDEIIEQISE | | | | |
| | | FSKRVILADANLDKVLSAYNKHRDKPIREQAENIIHLFTLTNL | | | | |
| | | GVPAAFKYFDTTIDKKRYTSTKEVLDATLIHQSITGLYETRIDL | | | | |
| | | SQLGGD | | | | |
| Spy Cas9- | Streptococcus | MDKKYSIGLDIGTNSVGWAVITDEYKVPSKKFKVLGNTDRHS | 9,025 | N863A | H840A | D10A |
| 3var- | pyogenes | IKKNLIGALLFDSGETAEATRLKRTARRRYTRRKNRICYLQEIF | | | | |
| NRTH | | SNEMAKVDDSFFHRLEESFLVEEDKKHERHPIFGNIVDEVAY | | | | |
| | | HEKYPTIYHLRKKLVDSTDKADLRLIYLALAHMIKFRGHFLIE | | | | |
| | | GDLNPDNSDVDKLFIQLVQTYNQLFEENPINASGVDAKAILSA | | | | |
| | | RLSKSRRLENLIAQLPGEKKNGLFGNLIALSLGLTPNFKSNFDL | | | | |
| | | AEDAKLQLSKDTYDDDLDNLLAQIGDQYADLFLAAKNLSDA | | | | |
| | | ILLSDILRVNTEITKAPLSASMVKRYDEHHQDLTLLKALVRQQ | | | | |
| | | LPEKYKEIFFDQSKNGYAGYIDGGASQEEFYKFIKPILEKMDG | | | | |
| | | TEELLVKLNREDLLRKQRTFDNGIIPHQIHLGELHAILRRQGDF | | | | |
| | | YPFLKDNREKIEKILTFRIPYYVGPLARGNSRFAWMTRKSEETI | | | | |
| | | TPWNFEEVVDKGASAQSFIERMTNFDKNLPNEKVLPKHSLLY | | | | |
| | | EYFTVYNELTKVKYVTEGMRKPAFLSGEQKKAIVDLLFKTNR | | | | |
| | | KVTVKQLKEDYFKKIECFDSVEISGVEDRFNASLGTYHDLLKI | | | | |
| | | IKDKDFLDNEENEDILEDIVLTLTLFEDREMIEERLKTYAHLFD | | | | |
| | | DKVMKQLKRLRYTGWGRLSRKLINGIRDKQSGKTILDFLKSD | | | | |
| | | GFANRNFMQLIHDDSLTFKEDIQKAQVSGQGDSLHEHIANLA | | | | |
| | | GSPAIKKGILQTVKVVDELVKVMGGHKPENIVIEMARENQTT | | | | |
| | | QKGQKNSRERMKRIEEGIKELGSQILKEHPVENTQLQNEKLY | | | | |
| | | LYYLQNGRDMYVDQELDINRLSDYDVDHIVPQSFLKDDSIDN | | | | |
| | | KVLTRSDKNRGKSDNVPSEEVVKKMKNYWRQLLNAKLITQR | | | | |
| | | KFDNLTKAERGGLSELDKAGFIKRQLVETRQITKHVAQILDSR | | | | |
| | | MNTKYDENDKLIREVKVITLKSKLVSDFRKDFQFYKVREINN | | | | |
| | | YHHAHDAYLNAVVGTALIKKYPKLESEFVYGDYKVYDVRK | | | | |
| | | MIAKSEQEIGKATAKYFFYSNIMNFFKTEITLANGEIRKRPLIE | | | | |
| | | TNGETGEIVWDKGRDFATVRKVLSMPQVNIVKKTEVQTGGF | | | | |
| | | SKESILPKGNSDKLIARKKDWDPKKYGGFNSPTVAYSVLVVA | | | | |
| | | KVEKGKSKKLKSVKELLGITIMERSSFEKNPIGFLEAKGYKEV | | | | |
| | | KKDLIIKLPKYSLFELENGRKRMLASASVLHKGNELALPSKY | | | | |
| | | VNFLYLASHYEKLKGSSEDNKQKQLFVEQHKHYLDEIIEQISE | | | | |
| | | FSKRVILADANLDKVLSAYNKHRDKPIREQAENIIHLFTLTNL | | | | |
| | | GASAAFKYFDTTIGRKLYTSTKEVLDATLIHQSITGLYETRIDL | | | | |
| | | SQLGGD | | | | |
| Spy Cas9- | Streptococcus | MDKKYSIGLDIGTNSVGWAVITDEYKVPSKKFKVLGNTDRHS | 9,026 | N863A | H840A | D10A |
| 3var- | pyogenes | IKKNLIGALLFDSGETAEATRLKRTARRRYTRRKNRICYLQEIF | | | | |
| NRCH | | SNEMAKVDDSFFHRLEESFLVEEDKKHERHPIFGNIVDEVAY | | | | |
| | | HEKYPTIYHLRKKLVDSTDKADLRLIYLALAHMIKFRGHFLIE | | | | |
| | | GDLNPDNSDVDKLFIQLVQTYNQLFEENPINASGVDAKAILSA | | | | |
| | | RLSKSRRLENLIAQLPGEKKNGLFGNLIALSLGLTPNFKSNFDL | | | | |
| | | AEDAKLQLSKDTYDDDLDNLLAQIGDQYADLFLAAKNLSDA | | | | |
| | | ILLSDILRVNTEITKAPLSASMVKRYDEHHQDLTLLKALVRQQ | | | | |
| | | LPEKYKEIFFDQSKNGYAGYIDGGASQEEFYKFIKPILEKMDG | | | | |
| | | TEELLVKLNREDLLRKQRTFDNGIIPHQIHLGELHAILRRQGDF | | | | |
| | | YPFLKDNREKIEKILTFRIPYYVGPLARGNSRFAWMTRKSEETI | | | | |
| | | TPWNFEEVVDKGASAQSFIERMTNFDKNLPNEKVLPKHSLLY | | | | |
| | | EYFTVYNELTKVKYVTEGMRKPAFLSGEQKKAIVDLLFKTNR | | | | |
| | | KVTVKQLKEDYFKKIECFDSVEISGVEDRFNASLGTYHDLLKI | | | | |
| | | IKDKDFLDNEENEDILEDIVLTLTLFEDREMIEERLKTYAHLFD | | | | |
| | | DKVMKQLKRLRYTGWGRLSRKLINGIRDKQSGKTILDFLKSD | | | | |
| | | GFANRNFMQLIHDDSLTFKEDIQKAQVSGQGDSLHEHIANLA | | | | |
| | | GSPAIKKGILQTVKVVDELVKVMGGHKPENIVIEMARENQTT | | | | |
| | | QKGQKNSRERMKRIEEGIKELGSQILKEHPVENTQLQNEKLY | | | | |
| | | LYYLQNGRDMYVDQELDINRLSDYDVDHIVPQSFLKDDSIDN | | | | |
| | | KVLTRSDKNRGKSDNVPSEEVVKKMKNYWRQLLNAKLITQR | | | | |
| | | KFDNLTKAERGGLSELDKAGFIKRQLVETRQITKHVAQILDSR | | | | |
| | | MNTKYDENDKLIREVKVITLKSKLVSDFRKDFQFYKVREINN | | | | |
| | | YHHAHDAYLNAVVGTALIKKYPKLESEFVYGDYKVYDVRK | | | | |
| | | MIAKSEQEIGKATAKYFFYSNIMNFFKTEITLANGEIRKRPLIE | | | | |
| | | TNGETGEIVWDKGRDFATVRKVLSMPQVNIVKKTEVQTGGF | | | | |
| | | SKESILPKGNSDKLIARKKDWDPKKYGGFNSPTVAYSVLVVA | | | | |
| | | KVEKGKSKKLKSVKELLGITIMERSSFEKNPIDFLEAKGYKEV | | | | |
| | | KKDLIIKLPKYSLFELENGRKRMLASAGVLQKGNELALPSKY | | | | |
| | | VNFLYLASHYEKLKGSPEDNEQKQLFVEQHKHYLDEIIEQISE | | | | |
| | | FSKRVILADANLDKVLSAYNKHRDKPIREQAENIIHLFTLTNL | | | | |
| | | GAPAAFKYFDTTINRKQYNTTKEVLDATLIRQSITGLYETRID | | | | |
| | | LSQLGGD | | | | |
| SpyCas9- | Streptococcus | MDKKYSIGLDIGTNSVGWAVITDEYKVPSKKFKVLGNTDRHS | 9,027 | N863A | H840A | D10A |
| HF1 | pyogenes | IKKNLIGALLFDSGETAEATRLKRTARRRYTRRKNRICYLQEIF | | | | |
| | | SNEMAKVDDSFFHRLEESFLVEEDKKHERHPIFGNIVDEVAY | | | | |
| | | HEKYPTIYHLRKKLVDSTDKADLRLIYLALAHMIKFRGHFLIE | | | | |
| | | GDLNPDNSDVDKLFIQLVQTYNQLFEENPINASGVDAKAILSA | | | | |
| | | RLSKSRRLENLIAQLPGEKKNGLFGNLIALSLGLTPNFKSNFDL | | | | |
| | | AEDAKLQLSKDTYDDDLDNLLAQIGDQYADLFLAAKNLSDA | | | | |
| | | ILLSDILRVNTEITKAPLSASMIKRYDEHHQDLTLLKALVRQQ | | | | |
| | | LPEKYKEIFFDQSKNGYAGYIDGGASQEEFYKFIKPILEKMDG | | | | |
| | | TEELLVKLNREDLLRKQRTFDNGSIPHQIHLGELHAILRRQED | | | | |
| | | FYPFLKDNREKIEKILTFRIPYYVGPLARGNSRFAWMTRKSEE | | | | |
| | | TITPWNFEEVVDKGASAQSFIERMTNFDKNLPNEKVLPKHSLL | | | | |
| | | YEYFTVYNELTKVKYVTEGMRKPAFLSGEQKKAIVDLLFKTN | | | | |
| | | RKVTVKQLKEDYFKKIECFDSVEISGVEDRFNASLGTYHDLL | | | | |
| | | KIIKDKDFLDNEENEDILEDIVLTLTLFEDREMIEERLKTYAHL | | | | |
| | | FDDKVMKQLKRRRYTGWGRLSRKLINGIRDKQSGKTILDFLK | | | | |
| | | SDGFANRNFMQLIHDDSLTFKEDIQKAQVSGQGDSLHEHIAN | | | | |
| | | LAGSPAIKKGILQTVKVVDELVKVMGRHKPENIVIEMARENQ | | | | |
| | | TTQKGQKNSRERMKRIEEGIKELGSQILKEHPVENTQLQNEKL | | | | |
| | | YLYYLQNGRDMYVDQELDINRLSDYDVDHIVPQSFLKDDSID | | | | |
| | | NKVLTRSDKNRGKSDNVPSEEVVKKMKNYWRQLLNAKLITQ | | | | |
| | | RKFDNLTKAERGGLSELDKAGFIKRQLVETRQITKHVAQILDS | | | | |
| | | RMNTKYDENDKLIREVKVITLKSKLVSDFRKDFQFYKVREIN | | | | |
| | | NYHHAHDAYLNAVVGTALIKKYPKLESEFVYGDYKVYDVR | | | | |
| | | KMIAKSEQEIGKATAKYFFYSNIMNFFKTEITLANGEIRKRPLI | | | | |
| | | ETNGETGEIVWDKGRDFATVRKVLSMPQVNIVKKTEVQTGG | | | | |
| | | FSKESILPKRNSDKLIARKKDWDPKKYGGFDSPTVAYSVLVV | | | | |
| | | AKVEKGKSKKLKSVKELLGITIMERSSFEKNPIDFLEAKGYKE | | | | |
| | | VKKDLIIKLPKYSLFELENGRKRMLASAGELQKGNELALPSK | | | | |
| | | YVNFLYLASHYEKLKGSPEDNEQKQLFVEQHKHYLDEIIEQIS | | | | |
| | | EFSKRVILADANLDKVLSAYNKHRDKPIREQAENIIHLFTLTN | | | | |
| | | LGAPAAFKYFDTTIDRKRYTSTKEVLDATLIHQSITGLYETRID | | | | |
| | | LSQLGGD | | | | |
| SpyCas9- | Streptococcus | MDKKYSIGLDIGTNSVGWAVITDEYKVPSKKFKVLGNTDRHS | 9,028 | N863A | H840A | D10A |
| QQR1 | pyogenes | IKKNLIGALLFDSGETAEATRLKRTARRRYTRRKNRICYLQEIF | | | | |
| | | SNEMAKVDDSFFHRLEESFLVEEDKKHERHPIFGNIVDEVAY | | | | |
| | | HEKYPTIYHLRKKLVDSTDKADLRLIYLALAHMIKFRGHFLIE | | | | |
| | | GDLNPDNSDVDKLFIQLVQTYNQLFEENPINASGVDAKAILSA | | | | |
| | | RLSKSRRLENLIAQLPGEKKNGLFGNLIALSLGLTPNFKSNFDL | | | | |
| | | AEDAKLQLSKDTYDDDLDNLLAQIGDQYADLFLAAKNLSDA | | | | |
| | | ILLSDILRVNTEITKAPLSASMIKRYDEHHQDLTLLKALVRQQ | | | | |
| | | LPEKYKEIFFDQSKNGYAGYIDGGASQEEFYKFIKPILEKMDG | | | | |
| | | TEELLVKLNREDLLRKQRTFDNGSIPHQIHLGELHAILRRQED | | | | |
| | | FYPFLKDNREKIEKILTFRIPYYVGPLARGNSRFAWMTRKSEE | | | | |
| | | TITPWNFEEVVDKGASAQSFIERMTNFDKNLPNEKVLPKHSLL | | | | |
| | | YEYFTVYNELTKVKYVTEGMRKPAFLSGEQKKAIVDLLFKTN | | | | |
| | | RKVTVKQLKEDYFKKIECFDSVEISGVEDRFNASLGTYHDLL | | | | |
| | | KIIKDKDFLDNEENEDILEDIVLTLTLFEDREMIEERLKTYAHL | | | | |
| | | FDDKVMKQLKRRRYTGWGRLSRKLINGIRDKQSGKTILDFLK | | | | |
| | | SDGFANRNFMQLIHDDSLTFKEDIQKAQVSGQGDSLHEHIAN | | | | |
| | | LAGSPAIKKGILQTVKVVDELVKVMGRHKPENIVIEMARENQ | | | | |
| | | TTQKGQKNSRERMKRIEEGIKELGSQILKEHPVENTQLQNEKL | | | | |
| | | YLYYLQNGRDMYVDQELDINRLSDYDVDHIVPQSFLKDDSID | | | | |
| | | NKVLTRSDKNRGKSDNVPSEEVVKKMKNYWRQLLNAKLITQ | | | | |
| | | RKFDNLTKAERGGLSELDKAGFIKRQLVETRQITKHVAQILDS | | | | |
| | | RMNTKYDENDKLIREVKVITLKSKLVSDFRKDFQFYKVREIN | | | | |
| | | NYHHAHDAYLNAVVGTALIKKYPKLESEFVYGDYKVYDVR | | | | |
| | | KMIAKSEQEIGKATAKYFFYSNIMNFFKTEITLANGEIRKRPLI | | | | |
| | | ETNGETGEIVWDKGRDFATVRKVLSMPQVNIVKKTEVQTGG | | | | |
| | | FSKESILPKRNSDKLIARKKDWDPKKYGGFDSPTVAYSVLVV | | | | |
| | | AKVEKGKSKKLKSVKELLGITIMERSSFEKNPIDFLEAKGYKE | | | | |
| | | VKKDLIIKLPKYSLFELENGRKRMLASARELQKGNELALPSK | | | | |
| | | YVNFLYLASHYEKLKGSPEDNEQKQLFVEQHKHYLDEIIEQIS | | | | |
| | | EFSKRVILADAQLDKVLSAYNKHRDKPIREQAENIIHLFTLTN | | | | |
| | | LGAPAAFKYFDTTFKQKQYRSTKEVLDATLIHQSITGLYETRI | | | | |
| | | DLSQLGGD | | | | |
| SpyCas9- | Streptococcus | MDKKYSIGLDIGTNSVGWAVITDEYKVPSKKFKVLGNTDRHS | 9,029 | N863A | H840A | D10A |
| SpG | pyogenes | IKKNLIGALLFDSGETAEATRLKRTARRRYTRRKNRICYLQEIF | | | | |
| | | SNEMAKVDDSFFHRLEESFLVEEDKKHERHPIFGNIVDEVAY | | | | |
| | | HEKYPTIYHLRKKLVDSTDKADLRLIYLALAHMIKFRGHFLIE | | | | |
| | | GDLNPDNSDVDKLFIQLVQTYNQLFEENPINASGVDAKAILSA | | | | |
| | | RLSKSRRLENLIAQLPGEKKNGLFGNLIALSLGLTPNFKSNFDL | | | | |
| | | AEDAKLQLSKDTYDDDLDNLLAQIGDQYADLFLAAKNLSDA | | | | |
| | | ILLSDILRVNTEITKAPLSASMIKRYDEHHQDLTLLKALVRQQ | | | | |
| | | LPEKYKEIFFDQSKNGYAGYIDGGASQEEFYKFIKPILEKMDG | | | | |
| | | TEELLVKLNREDLLRKQRTFDNGSIPHQIHLGELHAILRRQED | | | | |
| | | FYPFLKDNREKIEKILTFRIPYYVGPLARGNSRFAWMTRKSEE | | | | |
| | | TITPWNFEEVVDKGASAQSFIERMTNFDKNLPNEKVLPKHSLL | | | | |
| | | YEYFTVYNELTKVKYVTEGMRKPAFLSGEQKKAIVDLLFKTN | | | | |
| | | RKVTVKQLKEDYFKKIECFDSVEISGVEDRFNASLGTYHDLL | | | | |
| | | KIIKDKDFLDNEENEDILEDIVLTLTLFEDREMIEERLKTYAHL | | | | |
| | | FDDKVMKQLKRRRYTGWGRLSRKLINGIRDKQSGKTILDFLK | | | | |
| | | SDGFANRNFMQLIHDDSLTFKEDIQKAQVSGQGDSLHEHIAN | | | | |
| | | LAGSPAIKKGILQTVKVVDELVKVMGRHKPENIVIEMARENQ | | | | |
| | | TTQKGQKNSRERMKRIEEGIKELGSQILKEHPVENTQLQNEKL | | | | |
| | | YLYYLQNGRDMYVDQELDINRLSDYDVDHIVPQSFLKDDSID | | | | |
| | | NKVLTRSDKNRGKSDNVPSEEVVKKMKNYWRQLLNAKLITQ | | | | |
| | | RKFDNLTKAERGGLSELDKAGFIKRQLVETRQITKHVAQILDS | | | | |
| | | RMNTKYDENDKLIREVKVITLKSKLVSDFRKDFQFYKVREIN | | | | |
| | | NYHHAHDAYLNAVVGTALIKKYPKLESEFVYGDYKVYDVR | | | | |
| | | KMIAKSEQEIGKATAKYFFYSNIMNFFKTEITLANGEIRKRPLI | | | | |
| | | ETNGETGEIVWDKGRDFATVRKVLSMPQVNIVKKTEVQTGG | | | | |
| | | FSKESILPKRNSDKLIARKKDWDPKKYGGFLWPTVAYSVLVV | | | | |
| | | AKVEKGKSKKLKSVKELLGITIMERSSFEKNPIDFLEAKGYKE | | | | |
| | | VKKDLIIKLPKYSLFELENGRKRMLASAKQLQKGNELALPSK | | | | |
| | | YVNFLYLASHYEKLKGSPEDNEQKQLFVEQHKHYLDEIIEQIS | | | | |
| | | EFSKRVILADANLDKVLSAYNKHRDKPIREQAENIIHLFTLTN | | | | |
| | | LGAPAAFKYFDTTIDRKQYRSTKEVLDATLIHQSITGLYETRID | | | | |
| | | LSQLGGD | | | | |
| SpyCas9- | Streptococcus | MDKKYSIGLDIGTNSVGWAVITDEYKVPSKKFKVLGNTDRHS | 9,030 | N863A | H840A | D10A |
| VQR | pyogenes | IKKNLIGALLFDSGETAEATRLKRTARRRYTRRKNRICYLQEIF | | | | |
| | | SNEMAKVDDSFFHRLEESFLVEEDKKHERHPIFGNIVDEVAY | | | | |
| | | HEKYPTIYHLRKKLVDSTDKADLRLIYLALAHMIKFRGHFLIE | | | | |
| | | GDLNPDNSDVDKLFIQLVQTYNQLFEENPINASGVDAKAILSA | | | | |
| | | RLSKSRRLENLIAQLPGEKKNGLFGNLIALSLGLTPNFKSNFDL | | | | |
| | | AEDAKLQLSKDTYDDDLDNLLAQIGDQYADLFLAAKNLSDA | | | | |
| | | ILLSDILRVNTEITKAPLSASMIKRYDEHHQDLTLLKALVRQQ | | | | |
| | | LPEKYKEIFFDQSKNGYAGYIDGGASQEEFYKFIKPILEKMDG | | | | |
| | | TEELLVKLNREDLLRKQRTFDNGSIPHQIHLGELHAILRRQED | | | | |
| | | FYPFLKDNREKIEKILTFRIPYYVGPLARGNSRFAWMTRKSEE | | | | |
| | | TITPWNFEEVVDKGASAQSFIERMTNFDKNLPNEKVLPKHSLL | | | | |
| | | YEYFTVYNELTKVKYVTEGMRKPAFLSGEQKKAIVDLLFKTN | | | | |
| | | RKVTVKQLKEDYFKKIECFDSVEISGVEDRFNASLGTYHDLL | | | | |
| | | KIIKDKDFLDNEENEDILEDIVLTLTLFEDREMIEERLKTYAHL | | | | |
| | | FDDKVMKQLKRRRYTGWGRLSRKLINGIRDKQSGKTILDFLK | | | | |
| | | SDGFANRNFMQLIHDDSLTFKEDIQKAQVSGQGDSLHEHIAN | | | | |
| | | LAGSPAIKKGILQTVKVVDELVKVMGRHKPENIVIEMARENQ | | | | |
| | | TTQKGQKNSRERMKRIEEGIKELGSQILKEHPVENTQLQNEKL | | | | |
| | | YLYYLQNGRDMYVDQELDINRLSDYDVDHIVPQSFLKDDSID | | | | |
| | | NKVLTRSDKNRGKSDNVPSEEVVKKMKNYWRQLLNAKLITQ | | | | |
| | | RKFDNLTKAERGGLSELDKAGFIKRQLVETRQITKHVAQILDS | | | | |
| | | RMNTKYDENDKLIREVKVITLKSKLVSDFRKDFQFYKVREIN | | | | |
| | | NYHHAHDAYLNAVVGTALIKKYPKLESEFVYGDYKVYDVR | | | | |
| | | KMIAKSEQEIGKATAKYFFYSNIMNFFKTEITLANGEIRKRPLI | | | | |
| | | ETNGETGEIVWDKGRDFATVRKVLSMPQVNIVKKTEVQTGG | | | | |
| | | FSKESILPKRNSDKLIARKKDWDPKKYGGFVSPTVAYSVLVV | | | | |
| | | AKVEKGKSKKLKSVKELLGITIMERSSFEKNPIDFLEAKGYKE | | | | |
| | | VKKDLIIKLPKYSLFELENGRKRMLASAGELQKGNELALPSK | | | | |
| | | YVNFLYLASHYEKLKGSPEDNEQKQLFVEQHKHYLDEIIEQIS | | | | |
| | | EFSKRVILADANLDKVLSAYNKHRDKPIREQAENIIHLFTLTN | | | | |
| | | LGAPAAFKYFDTTIDRKQYRSTKEVLDATLIHQSITGLYETRID | | | | |
| | | LSQLGGD | | | | |
| SpyCas9- | Streptococcus | MDKKYSIGLDIGTNSVGWAVITDEYKVPSKKFKVLGNTDRHS | 9,031 | N863A | H840A | D10A |
| VRER | pyogenes | IKKNLIGALLFDSGETAEATRLKRTARRRYTRRKNRICYLQEIF | | | | |
| | | SNEMAKVDDSFFHRLEESFLVEEDKKHERHPIFGNIVDEVAY | | | | |
| | | HEKYPTIYHLRKKLVDSTDKADLRLIYLALAHMIKFRGHFLIE | | | | |
| | | GDLNPDNSDVDKLFIQLVQTYNQLFEENPINASGVDAKAILSA | | | | |
| | | RLSKSRRLENLIAQLPGEKKNGLFGNLIALSLGLTPNFKSNFDL | | | | |
| | | AEDAKLQLSKDTYDDDLDNLLAQIGDQYADLFLAAKNLSDA | | | | |
| | | ILLSDILRVNTEITKAPLSASMIKRYDEHHQDLTLLKALVRQQ | | | | |
| | | LPEKYKEIFFDQSKNGYAGYIDGGASQEEFYKFIKPILEKMDG | | | | |
| | | TEELLVKLNREDLLRKQRTFDNGSIPHQIHLGELHAILRRQED | | | | |
| | | FYPFLKDNREKIEKILTFRIPYYVGPLARGNSRFAWMTRKSEE | | | | |
| | | TITPWNFEEVVDKGASAQSFIERMTNFDKNLPNEKVLPKHSLL | | | | |
| | | YEYFTVYNELTKVKYVTEGMRKPAFLSGEQKKAIVDLLFKTN | | | | |
| | | RKVTVKQLKEDYFKKIECFDSVEISGVEDRFNASLGTYHDLL | | | | |
| | | KIIKDKDFLDNEENEDILEDIVLTLTLFEDREMIEERLKTYAHL | | | | |
| | | FDDKVMKQLKRRRYTGWGRLSRKLINGIRDKQSGKTILDFLK | | | | |
| | | SDGFANRNFMQLIHDDSLTFKEDIQKAQVSGQGDSLHEHIAN | | | | |
| | | LAGSPAIKKGILQTVKVVDELVKVMGRHKPENIVIEMARENQ | | | | |
| | | TTQKGQKNSRERMKRIEEGIKELGSQILKEHPVENTQLQNEKL | | | | |
| | | YLYYLQNGRDMYVDQELDINRLSDYDVDHIVPQSFLKDDSID | | | | |
| | | NKVLTRSDKNRGKSDNVPSEEVVKKMKNYWRQLLNAKLITQ | | | | |
| | | RKFDNLTKAERGGLSELDKAGFIKRQLVETRQITKHVAQILDS | | | | |
| | | RMNTKYDENDKLIREVKVITLKSKLVSDFRKDFQFYKVREIN | | | | |
| | | NYHHAHDAYLNAVVGTALIKKYPKLESEFVYGDYKVYDVR | | | | |
| | | KMIAKSEQEIGKATAKYFFYSNIMNFFKTEITLANGEIRKRPLI | | | | |
| | | ETNGETGEIVWDKGRDFATVRKVLSMPQVNIVKKTEVQTGG | | | | |
| | | FSKESILPKRNSDKLIARKKDWDPKKYGGFVSPTVAYSVLVV | | | | |
| | | AKVEKGKSKKLKSVKELLGITIMERSSFEKNPIDFLEAKGYKE | | | | |
| | | VKKDLIIKLPKYSLFELENGRKRMLASARELQKGNELALPSK | | | | |
| | | YVNFLYLASHYEKLKGSPEDNEQKQLFVEQHKHYLDEIIEQIS | | | | |
| | | EFSKRVILADANLDKVLSAYNKHRDKPIREQAENIIHLFTLTN | | | | |
| | | LGAPAAFKYFDTTIDRKEYRSTKEVLDATLIHQSITGLYETRID | | | | |
| | | LSQLGGD | | | | |
| SpyCas9- | Streptococcus | MDKKYSIGLDIGTNSVGWAVITDEYKVPSKKFKVLGNTDRHS | 9,032 | N863A | H840A | D10A |
| xCas | pyogenes | IKKNLIGALLFDSGETAEATRLKRTARRRYTRRKNRICYLQEIF | | | | |
| | | SNEMAKVDDSFFHRLEESFLVEEDKKHERHPIFGNIVDEVAY | | | | |
| | | HEKYPTIYHLRKKLVDSTDKADLRLIYLALAHMIKFRGHFLIE | | | | |
| | | GDLNPDNSDVDKLFIQLVQTYNQLFEENPINASGVDAKAILSA | | | | |
| | | RLSKSRRLENLIAQLPGEKKNGLFGNLIALSLGLTPNFKSNFDL | | | | |
| | | AEDTKLQLSKDTYDDDLDNLLAQIGDQYADLFLAAKNLSDAI | | | | |
| | | LLSDILRVNTEITKAPLSASMIKLYDEHHQDLTLLKALVRQQL | | | | |
| | | PEKYKEIFFDQSKNGYAGYIDGGASQEEFYKFIKPILEKMDGT | | | | |
| | | EELLVKLNREDLLRKQRTFDNGIIPHQIHLGELHAILRRQEDFY | | | | |
| | | PFLKDNREKIEKILTFRIPYYVGPLARGNSRFAWMTRKSEETIT | | | | |
| | | PWNFEKVVDKGASAQSFIERMTNFDKNLPNEKVLPKHSLLYE | | | | |
| | | YFTVYNELTKVKYVTEGMRKPAFLSGDQKKAIVDLLFKTNR | | | | |
| | | KVTVKQLKEDYFKKIECFDSVEISGVEDRFNASLGTYHDLLKI | | | | |
| | | IKDKDFLDNEENEDILEDIVLTLTLFEDREMIEERLKTYAHLFD | | | | |
| | | DKVMKQLKRRRYTGWGRLSRKLINGIRDKQSGKTILDFLKSD | | | | |
| | | GFANRNFIQLIHDDSLTFKEDIQKAQVSGQGDSLHEHIANLAG | | | | |
| | | SPAIKKGILQTVKVVDELVKVMGRHKPENIVIEMARENQTTQ | | | | |
| | | KGQKNSRERMKRIEEGIKELGSQILKEHPVENTQLQNEKLYL | | | | |
| | | YYLQNGRDMYVDQELDINRLSDYDVDHIVPQSFLKDDSIDNK | | | | |
| | | VLTRSDKNRGKSDNVPSEEVVKKMKNYWRQLLNAKLITQRK | | | | |
| | | FDNLTKAERGGLSELDKAGFIKRQLVETRQITKHVAQILDSR | | | | |
| | | MNTKYDENDKLIREVKVITLKSKLVSDFRKDFQFYKVREINN | | | | |
| | | YHHAHDAYLNAVVGTALIKKYPKLESEFVYGDYKVYDVRK | | | | |
| | | MIAKSEQEIGKATAKYFFYSNIMNFFKTEITLANGEIRKRPLIE | | | | |
| | | TNGETGEIVWDKGRDFATVRKVLSMPQVNIVKKTEVQTGGF | | | | |
| | | SKESILPKRNSDKLIARKKDWDPKKYGGFDSPTVAYSVLVVA | | | | |
| | | KVEKGKSKKLKSVKELLGITIMERSSFEKNPIDFLEAKGYKEV | | | | |
| | | KKDLIIKLPKYSLFELENGRKRMLASAGVLQKGNELALPSKY | | | | |
| | | VNFLYLASHYEKLKGSPEDNEQKQLFVEQHKHYLDEIIEQISE | | | | |
| | | FSKRVILADANLDKVLSAYNKHRDKPIREQAENIIHLFTLTNL | | | | |
| | | GAPAAFKYFDTTIDRKRYTSTKEVLDATLIHQSITGLYETRIDL | | | | |
| | | SQLGGD | | | | |
| SpyCas9- | Streptococcus | MDKKYSIGLDIGTNSVGWAVITDEYKVPSKKFKVLGNTDRHS | 9,033 | N863A | H840A | D10A |
| xCas-NG | pyogenes | IKKNLIGALLFDSGETAEATRLKRTARRRYTRRKNRICYLQEIF | | | | |
| | | SNEMAKVDDSFFHRLEESFLVEEDKKHERHPIFGNIVDEVAY | | | | |
| | | HEKYPTIYHLRKKLVDSTDKADLRLIYLALAHMIKFRGHFLIE | | | | |
| | | GDLNPDNSDVDKLFIQLVQTYNQLFEENPINASGVDAKAILSA | | | | |
| | | RLSKSRRLENLIAQLPGEKKNGLFGNLIALSLGLTPNFKSNFDL | | | | |
| | | AEDTKLQLSKDTYDDDLDNLLAQIGDQYADLFLAAKNLSDAI | | | | |
| | | LLSDILRVNTEITKAPLSASMIKLYDEHHQDLTLLKALVRQQL | | | | |
| | | PEKYKEIFFDQSKNGYAGYIDGGASQEEFYKFIKPILEKMDGT | | | | |
| | | EELLVKLNREDLLRKQRTFDNGIIPHQIHLGELHAILRRQEDFY | | | | |
| | | PFLKDNREKIEKILTFRIPYYVGPLARGNSRFAWMTRKSEETIT | | | | |
| | | PWNFEKVVDKGASAQSFIERMTNFDKNLPNEKVLPKHSLLYE | | | | |
| | | YFTVYNELTKVKYVTEGMRKPAFLSGDQKKAIVDLLFKTNR | | | | |
| | | KVTVKQLKEDYFKKIECFDSVEISGVEDRFNASLGTYHDLLKI | | | | |
| | | IKDKDFLDNEENEDILEDIVLTLTLFEDREMIEERLKTYAHLFD | | | | |
| | | DKVMKQLKRRRYTGWGRLSRKLINGIRDKQSGKTILDFLKSD | | | | |
| | | GFANRNFIQLIHDDSLTFKEDIQKAQVSGQGDSLHEHIANLAG | | | | |
| | | SPAIKKGILQTVKVVDELVKVMGRHKPENIVIEMARENQTTQ | | | | |
| | | KGQKNSRERMKRIEEGIKELGSQILKEHPVENTQLQNEKLYL | | | | |
| | | YYLQNGRDMYVDQELDINRLSDYDVDHIVPQSFLKDDSIDNK | | | | |
| | | VLTRSDKNRGKSDNVPSEEVVKKMKNYWRQLLNAKLITQRK | | | | |
| | | FDNLTKAERGGLSELDKAGFIKRQLVETRQITKHVAQILDSR | | | | |
| | | MNTKYDENDKLIREVKVITLKSKLVSDFRKDFQFYKVREINN | | | | |
| | | YHHAHDAYLNAVVGTALIKKYPKLESEFVYGDYKVYDVRK | | | | |
| | | MIAKSEQEIGKATAKYFFYSNIMNFFKTEITLANGEIRKRPLIE | | | | |
| | | TNGETGEIVWDKGRDFATVRKVLSMPQVNIVKKTEVQTGGF | | | | |
| | | SKESIRPKRNSDKLIARKKDWDPKKYGGFVSPTVAYSVLVVA | | | | |
| | | KVEKGKSKKLKSVKELLGITIMERSSFEKNPIDFLEAKGYKEV | | | | |
| | | KKDLIIKLPKYSLFELENGRKRMLASARFLQKGNELALPSKYV | | | | |
| | | NFLYLASHYEKLKGSPEDNEQKQLFVEQHKHYLDEIIEQISEF | | | | |
| | | SKRVILADANLDKVLSAYNKHRDKPIREQAENIIHLFTLTNLG | | | | |
| | | APRAFKYFDTTIDRKVYRSTKEVLDATLIHQSITGLYETRIDLS | | | | |
| | | QLGGD | | | | |
| St1Cas9- | Streptococcus | MSDLVLGLDIGIGSVGVGILNKVTGEIIHKNSRIFPAAQAENNL | 9,034 | N622A | H599A | D9A |
| CNRZ1066 | thermophilus | VRRTNRQGRRLARRKKHRRVRLNRLFEESGLITDFTKISINLN | | | | |
| | | PYQLRVKGLTDELSNEELFIALKNMVKHRGISYLDDASDDGN | | | | |
| | | SSVGDYAQIVKENSKQLETKTPGQIQLERYQTYGQLRGDFTV | | | | |
| | | EKDGKKHRLINVFPTSAYRSEALRILQTQQEFNPQITDEFINRY | | | | |
| | | LEILTGKRKYYHGPGNEKSRTDYGRYRTSGETLDNIFGILIGK | | | | |
| | | CTFYPDEFRAAKASYTAQEFNLLNDLNNLTVPTETKKLSKEQ | | | | |
| | | KNQIINYVKNEKAMGPAKLFKYIAKLLSCDVADIKGYRIDKS | | | | |
| | | GKAEIHTFEAYRKMKTLETLDIEQMDRETLDKLAYVLTLNTE | | | | |
| | | REGIQEALEHEFADGSFSQKQVDELVQFRKANSSIFGKGWHN | | | | |
| | | FSVKLMMELIPELYETSEEQMTILTRLGKQKTTSSSNKTKYID | | | | |
| | | EKLLTEEIYNPVVAKSVRQAIKIVNAAIKEYGDFDNIVIEMAR | | | | |
| | | ETNEDDEKKAIQKIQKANKDEKDAAMLKAANQYNGKAELP | | | | |
| | | HSVFHGHKQLATKIRLWHQQGERCLYTGKTISIHDLINNSNQF | | | | |
| | | EVDHILPLSITFDDSLANKVLVYATANQEKGQRTPYQALDSM | | | | |
| | | DDAWSFRELKAFVRESKTLSNKKKEYLLTEEDISKFDVRKKFI | | | | |
| | | ERNLVDTRYASRVVLNALQEHFRAHKIDTKVSVVRGQFTSQL | | | | |
| | | RRHWGIEKTRDTYHHHAVDALIIAASSQLNLWKKQKNTLVS | | | | |
| | | YSEEQLLDIETGELISDDEYKESVFKAPYQHFVDTLKSKEFED | | | | |
| | | SILFSYQVDSKFNRKISDATIYATRQAKVGKDKKDETYVLGKI | | | | |
| | | KDIYTQDGYDAFMKIYKKDKSKFLMYRHDPQTFEKVIEPILE | | | | |
| | | NYPNKQMNEKGKEVPCNPFLKYKEEHGYIRKYSKKGNGPEI | | | | |
| | | KSLKYYDSKLLGNPIDITPENSKNKVVLQSLKPWRTDVYFNK | | | | |
| | | ATGKYEILGLKYADLQFEKGTGTYKISQEKYNDIKKKEGVDS | | | | |
| | | DSEFKFTLYKNDLLLVKDTETKEQQLFRFLSRTLPKQKHYVE | | | | |
| | | LKPYDKQKFEGGEALIKVLGNVANGGQCIKGLAKSNISIYKV | | | | |
| | | RTDVLGNQHIIKNEGDKPKLDF | | | | |
| St1Cas9- | Streptococcus | MSDLVLGLDIGIGSVGVGILNKVTGEIIHKNSRIFPAAQAENNL | 9,035 | N622A | H599A | D9A |
| LMG1831 | thermophilus | VRRTNRQGRRLARRKKHRRVRLNRLFEESGLITDFTKISINLN | | | | |
| | | PYQLRVKGLTDELSNEELFIALKNMVKHRGISYLDDASDDGN | | | | |
| | | SSVGDYAQIVKENSKQLETKTPGQIQLERYQTYGQLRGDFTV | | | | |
| | | EKDGKKHRLINVFPTSAYRSEALRILQTQQEFNPQITDEFINRY | | | | |
| | | LEILTGKRKYYHGPGNEKSRTDYGRYRTSGETLDNIFGILIGK | | | | |
| | | CTFYPDEFRAAKASYTAQEFNLLNDLNNLTVPTETKKLSKEQ | | | | |
| | | KNQIINYVKNEKAMGPAKLFKYIAKLLSCDVADIKGYRIDKS | | | | |
| | | GKAEIHTFEAYRKMKTLETLDIEQMDRETLDKLAYVLTLNTE | | | | |
| | | REGIQEALEHEFADGSFSQKQVDELVQFRKANSSIFGKGWHN | | | | |
| | | FSVKLMMELIPELYETSEEQMTILTRLGKQKTTSSSNKTKYID | | | | |
| | | EKLLTEEIYNPVVAKSVRQAIKIVNAAIKEYGDFDNIVIEMAR | | | | |
| | | ETNEDDEKKAIQKIQKANKDEKDAAMLKAANQYNGKAELP | | | | |
| | | HSVFHGHKQLATKIRLWHQQGERCLYTGKTISIHDLINNSNQF | | | | |
| | | EVDHILPLSITFDDSLANKVLVYATANQEKGQRTPYQALDSM | | | | |
| | | DDAWSFRELKAFVRESKTLSNKKKEYLLTEEDISKFDVRKKFI | | | | |
| | | ERNLVDTRYASRVVLNALQEHFRAHKIDTKVSVVRGQFTSQL | | | | |
| | | RRHWGIEKTRDTYHHHAVDALIIAASSQLNLWKKQKNTLVS | | | | |
| | | YSEEQLLDIETGELISDDEYKESVFKAPYQHFVDTLKSKEFED | | | | |
| | | SILFSYQVDSKFNRKISDATIYATRQAKVGKDKKDETYVLGKI | | | | |
| | | KDIYTQDGYDAFMKIYKKDKSKFLMYRHDPQTFEKVIEPILE | | | | |
| | | NYPNKQMNEKGKEVPCNPFLKYKEEHGYIRKYSKKGNGPEI | | | | |
| | | KSLKYYDSKLLGNPIDITPENSKNKVVLQSLKPWRTDVYFNK | | | | |
| | | NTGKYEILGLKYADLQFEKKTGTYKISQEKYNGIMKEEGVDS | | | | |
| | | DSEFKFTLYKNDLLLVKDTETKEQQLFRFLSRTMPNVKYYVE | | | | |
| | | LKPYSKDKFEKNESLIEILGSADKSGRCIKGLGKSNISIYKVRT | | | | |
| | | DVLGNQHIIKNEGDKPKLDF | | | | |
| St1Cas9- | Streptococcus | MSDLVLGLDIGIGSVGVGILNKVTGEIIHKNSRIFPAAQAENNL | 9,036 | N622A | H599A | D9A |
| MTH17CL396 | thermophilus | VRRTNRQGRRLARRKKHRRVRLNRLFEESGLITDFTKISINLN | | | | |
| | | PYQLRVKGLTDELSNEELFIALKNMVKHRGISYLDDASDDGN | | | | |
| | | SSVGDYAQIVKENSKQLETKTPGQIQLERYQTYGQLRGDFTV | | | | |
| | | EKDGKKHRLINVFPTSAYRSEALRILQTQQEFNPQITDEFINRY | | | | |
| | | LEILTGKRKYYHGPGNEKSRTDYGRYRTSGETLDNIFGILIGK | | | | |
| | | CTFYPDEFRAAKASYTAQEFNLLNDLNNLTVPTETKKLSKEQ | | | | |
| | | KNQIINYVKNEKAMGPAKLFKYIAKLLSCDVADIKGYRIDKS | | | | |
| | | GKAEIHTFEAYRKMKTLETLDIEQMDRETLDKLAYVLTLNTE | | | | |
| | | REGIQEALEHEFADGSFSQKQVDELVQFRKANSSIFGKGWHN | | | | |
| | | FSVKLMMELIPELYETSEEQMTILTRLGKQKTTSSSNKTKYID | | | | |
| | | EKLLTEEIYNPVVAKSVRQAIKIVNAAIKEYGDFDNIVIEMAR | | | | |
| | | ETNEDDEKKAIQKIQKANKDEKDAAMLKAANQYNGKAELP | | | | |
| | | HSVFHGHKQLATKIRLWHQQGERCLYTGKTISIHDLINNSNQF | | | | |
| | | EVDHILPLSITFDDSLANKVLVYATANQEKGQRTPYQALDSM | | | | |
| | | DDAWSFRELKAFVRESKTLSNKKKEYLLTEEDISKFDVRKKFI | | | | |
| | | ERNLVDTRYASRVVLNALQEHFRAHKIDTKVSVVRGQFTSQL | | | | |
| | | RRHWGIEKTRDTYHHHAVDALIIAASSQLNLWKKQKNTLVS | | | | |
| | | YSEDQLLDIETGELISDDEYKESVFKAPYQHFVDTLKSKEFED | | | | |
| | | SILFSYQVDSKFNRKISDATIYATRQAKVGKDKADETYVLGKI | | | | |
| | | KDIYTQDGYDAFMKIYKKDKSKFLMYRHDPQTFEKVIEPILE | | | | |
| | | NYPNKQINEKGKEVPCNPFLKYKEEHGYIRKYSKKGNGPEIK | | | | |
| | | SLKYYDSKLGNHIDITPKDSNNKVVLQSLKPWRTDVYFNKNT | | | | |
| | | GKYEILGLKYSDMQFEKGTGKYSISKEQYENIKVREGVDENS | | | | |
| | | EFKFTLYKNDLLLLKDSENGEQILLRFTSRNDTSKHYVELKPY | | | | |
| | | NRQKFEGSEYLIKSLGTVAKGGQCIKGLGKSNISIYKVRTDVL | | | | |
| | | GNQHIIKNEGDKPKLDF | | | | |
| St1Cas9- | Streptococcus | MSDLVLGLDIGIGSVGVGILNKVTGEIIHKNSRIFPAAQAENNL | 9,037 | N622A | H599A | D9A |
| TH1477 | thermophilus | VRRTNRQGRRLARRKKHRRVRLNRLFEESGLITDFTKISINLN | | | | |
| | | PYQLRVKGLTDELSNEELFIALKNMVKHRGISYLDDASDDGN | | | | |
| | | SSVGDYAQIVKENSKQLETKTPGQIQLERYQTYGQLRGDFTV | | | | |
| | | EKDGKKHRLINVFPTSAYRSEALRILQTQQEFNPQITDEFINRY | | | | |
| | | LEILTGKRKYYHGPGNEKSRTDYGRYRTSGETLDNIFGILIGK | | | | |
| | | CTFYPDEFRAAKASYTAQEFNLLNDLNNLTVPTETKKLSKEQ | | | | |
| | | KNQIINYVKNEKAMGPAKLFKYIAKLLSCDVADIKGYRIDKS | | | | |
| | | GKAEIHTFEAYRKMKTLETLDIEQMDRETLDKLAYVLTLNTE | | | | |
| | | REGIQEALEHEFADGSFSQKQVDELVQFRKANSSIFGKGWHN | | | | |
| | | FSVKLMMELIPELYETSEEQMTILTRLGKQKTTSSSNKTKYID | | | | |
| | | EKLLTEEIYNPVVAKSVRQAIKIVNAAIKEYGDFDNIVIEMAR | | | | |
| | | ETNEDDEKKAIQKIQKANKDEKDAAMLKAANQYNGKAELP | | | | |
| | | HSVFHGHKQLATKIRLWHQQGERCLYTGKTISIHDLINNSNQF | | | | |
| | | EVDHILPLSITFDDSLANKVLVYATANQEKGQRTPYQALDSM | | | | |
| | | DDAWSFRELKAFVRESKTLSNKKKEYLLTEEDISKFDVRKKFI | | | | |
| | | ERNLVDTRYASRVVLNALQEHFRAHKIDTKVSVVRGQFTSQL | | | | |
| | | RRHWGIEKTRDTYHHHAVDALIIAASSQLNLWKKQKNTLVS | | | | |
| | | YSEDQLLDIETGELISDDEYKESVFKAPYQHFVDTLKSKEFED | | | | |
| | | SILFSYQVDSKFNRKISDATIYATRQAKVGKDKADETYVLGKI | | | | |
| | | KDIYTQDGYDAFMKIYKKDKSKFLMYRHDPQTFEKVIEPILE | | | | |
| | | NYPNKQINEKGKEVPCNPFLKYKEEHGYIRKYSKKGNGPEIK | | | | |
| | | SLKYYDSKLGNHIDITPKDSNNKVVLQSLKPWRTDVYFNKNT | | | | |
| | | GKYEILGLKYSDMQFEKGTGKYSISKEQYENIKVREGVDENS | | | | |
| | | EFKFTLYKNDLLLLKDSENGEQILLRFTSRNDTSKHYVELKPY | | | | |
| | | NRQKFEGSEYLIKSLGTVVKGGRCIKGLGKSNISIYKVRTDVL | | | | |
| | | GNQHIIKNEGDKPKLDF | | | | |
| SRGN3.1 | Staphylococcus | MNQKFILGLDIGITSVGYGLIDYETKNIIDAGVRLFPEANVENN | 9,038 | N585A | H562A | D10A |
| | spp. | EGRRSKRGSRRLKRRRIHRLERVKLLLTEYDLINKEQIPTSNNP | | | | |
| | | YQIRVKGLSEILSKDELAIALLHLAKRRGIHNVDVAADKEETA | | | | |
| | | SDSLSTKDQINKNAKFLESRYVCELQKERLENEGHVRGVENR | | | | |
| | | FLTKDIVREAKKIIDTQMQYYPEIDETFKEKYISLVETRREYFE | | | | |
| | | GPGQGSPFGWNGDLKKWYEMLMGHCTYFPQELRSVKYAYS | | | | |
| | | ADLFNALNDLNNLIIQRDNSEKLEYHEKYHIIENVFKQKKKPT | | | | |
| | | LKQIAKEIGVNPEDIKGYRITKSGTPEFTSFKLFHDLKKVVKD | | | | |
| | | HAILDDIDLLNQIAEILTIYQDKDSIVAELGQLEYLMSEADKQS | | | | |
| | | ISELTGYTGTHSLSLKCMNMIIDELWHSSMNQMEVFTYLNMR | | | | |
| | | PKKYELKGYQRIPTDMIDDAILSPVVKRTFIQSINVINKVIEKY | | | | |
| | | GIPEDIIIELARENNSDDRKKFINNLQKKNEATRKRINEIIGQTG | | | | |
| | | NQNAKRIVEKIRLHDQQEGKCLYSLESIPLEDLLNNPNHYEVD | | | | |
| | | HIIPRSVSFDNSYHNKVLVKQSENSKKSNLTPYQYFNSGKSKL | | | | |
| | | SYNQFKQHILNLSKSQDRISKKKKEYLLEERDINKFEVQKEFI | | | | |
| | | NRNLVDTRYATRELTNYLKAYFSANNMNVKVKTINGSFTDY | | | | |
| | | LRKVWKFKKERNHGYKHHAEDALIIANADFLFKENKKLKAV | | | | |
| | | NSVLEKPEIETKQLDIQVDSEDNYSEMFIIPKQVQDIKDFRNFK | | | | |
| | | YSHRVDKKPNRQLINDTLYSTRKKDNSTYIVQTIKDIYAKDNT | | | | |
| | | TLKKQFDKSPEKFLMYQHDPRTFEKLEVIMKQYANEKNPLA | | | | |
| | | KYHEETGEYLTKYSKKNNGPIVKSLKYIGNKLGSHLDVTHQF | | | | |
| | | KSSTKKLVKLSIKNYRFDVYLTEKGYKFVTIAYLNVFKKDNY | | | | |
| | | YYIPKDKYQELKEKKKIKDTDQFIASFYKNDLIKLNGDLYKII | | | | |
| | | GVNSDDRNIIELDYYDIKYKDYCEINNIKGEPRIKKTIGKKTES | | | | |
| | | IEKFTTDVLGNLYLHSTEKAPQLIFKRGL | | | | |
| SRGN3.3 | Staphylococcus | MNQKFILGLDIGITSVGYGLIDYETKNIIDAGVRLFPEANVENN | 9,039 | N585A | H562A | D10A |
| | spp. | EGRRSKRGSRRLKRRRIHRLERVKLLLTEYDLINKEQIPTSNNP | | | | |
| | | YQIRVKGLSEILSKDELAIALLHLAKRRGIHNVDVAADKEETA | | | | |
| | | SDSLSTKDQINKNAKFLESRYVCELQKERLENEGHVRGVENR | | | | |
| | | FLTKDIVREAKKIIDTQMQYYPEIDETFKEKYISLVETRREYFE | | | | |
| | | GPGQGSPFGWNGDLKKWYEMLMGHCTYFPQELRSVKYAYS | | | | |
| | | ADLFNALNDLNNLIIQRDNSEKLEYHEKYHIIENVFKQKKKPT | | | | |
| | | LKQIAKEIGVNPEDIKGYRITKSGTPEFTSFKLFHDLKKVVKD | | | | |
| | | HAILDDIDLLNQIAEILTIYQDKDSIVAELGQLEYLMSEADKQS | | | | |
| | | ISELTGYTGTHSLSLKCMNMIIDELWHSSMNQMEVFTYLNMR | | | | |
| | | PKKYELKGYQRIPTDMIDDAILSPVVKRTFIQSINVINKVIEKY | | | | |
| | | GIPEDIIIELARENNSDDRKKFINNLQKKNEATRKRINEIIGQTG | | | | |
| | | NQNAKRIVEKIRLHDQQEGKCLYSLESIPLEDLLNNPNHYEVD | | | | |
| | | HIIPRSVSFDNSYHNKVLVKQSENSKKSNLTPYQYFNSGKSKL | | | | |
| | | SYNQFKQHILNLSKSQDRISKKKKEYLLEERDINKFEVQKEFI | | | | |
| | | NRNLVDTRYATRELTSYLKAYFSANNMDVKVKTINGSFTNH | | | | |
| | | LRKVWRFDKYRNHGYKHHAEDALIIANADFLFKENKKLQNT | | | | |
| | | NKILEKPTIENNTKKVTVEKEEDYNNVFETPKLVEDIKQYRDY | | | | |
| | | KFSHRVDKKPNRQLINDTLYSTRMKDEHDYIVQTITDIYGKD | | | | |
| | | NTNLKKQFNKNPEKFLMYQNDPKTFEKLSIIMKQYSDEKNPL | | | | |
| | | AKYYEETGEYLTKYSKKNNGPIVKKIKLLGNKVGNHLDVTN | | | | |
| | | KYENSTKKLVKLSIKNYRFDVYLTEKGYKFVTIAYLNVFKKD | | | | |
| | | NYYYIPKDKYQELKEKKKIKDTDQFIASFYKNDLIKLNGDLY | | | | |
| | | KIIGVNSDDRNIIELDYYDIKYKDYCEINNIKGEPRIKKTIGKKT | | | | |
| | | ESIEKFTTDVLGNLYLHSTEKAPQLIFKRGL | | | | |

In some embodiments, a Cas protein requires a protospacer adjacent motif (PAM) to be present in or adjacent to a target DNA sequence for the Cas protein to bind and/or function. In some embodiments, the PAM is or comprises, from 5′ to 3′, NGG, YG, NNGRRT, NNNRRT, NGA, TYCV, TATV, NTTN, or NNNGATT, where N stands for any nucleotide, Y stands for C or T, R stands for A or G, and V stands for A or C or G. In some embodiments, a Cas protein is a protein listed in Table 7 or 8. In some embodiments, a Cas protein comprises one or more mutations altering its PAM. In some embodiments, a Cas protein comprises E1369R, E1449H, and R1556A mutations or analogous substitutions to the amino acids corresponding to said positions. In some embodiments, a Cas protein comprises E782K, N968K, and R1015H mutations or analogous substitutions to the amino acids corresponding to said positions. In some embodiments, a Cas protein comprises D1135V, R1335Q, and T1337R mutations or analogous substitutions to the amino acids corresponding to said positions. In some embodiments, a Cas protein comprises S542R and K607R mutations or analogous substitutions to the amino acids corresponding to said positions. In some embodiments, a Cas protein comprises S542R, K548V, and N552R mutations or analogous substitutions to the amino acids corresponding to said positions. Exemplary advances in the engineering of Cas enzymes to recognize altered PAM sequences are reviewed in Collias et al Nature Communications 12:555 (2021), incorporated herein by reference in its entirety.

In some embodiments, the Cas protein is catalytically active and cuts one or both strands of the target DNA site. In some embodiments, cutting the target DNA site is followed by formation of an alteration, e.g., an insertion or deletion, e.g., by the cellular repair machinery.

In some embodiments, the Cas protein is modified to deactivate or partially deactivate the nuclease, e.g., nuclease-deficient Cas9. Whereas wild-type Cas9 generates double-strand breaks (DSBs) at specific DNA sequences targeted by a gRNA, a number of CRISPR endonucleases having modified functionalities are available, for example: a “nickase” version of Cas9 that has been partially deactivated generates only a single-strand break; a catalytically inactive Cas9 (“dCas9”) does not cut target DNA. In some embodiments, dCas9 binding to a DNA sequence may interfere with transcription at that site by steric hindrance. In some embodiments, dCas9 binding to an anchor sequence may interfere with (e.g., decrease or prevent) genomic complex (e.g., ASMC) formation and/or maintenance. In some embodiments, a DNA-binding domain comprises a catalytically inactive Cas9, e.g., dCas9. Many catalytically inactive Cas9 proteins are known in the art. In some embodiments, dCas9 comprises mutations in each endonuclease domain of the Cas protein, e.g., D10A and H840A or N863A mutations. In some embodiments, a catalytically inactive or partially inactive CRISPR/Cas domain comprises a Cas protein comprising one or more mutations, e.g., one or more of the mutations listed in Table 7. In some embodiments, a Cas protein described on a given row of Table 7 comprises one, two, three, or all of the mutations listed in the same row of Table 7. In some embodiments, a Cas protein, e.g., not described in Table 7, comprises one, two, three, or all of the mutations listed in a row of Table 7 or a corresponding mutation at a corresponding site in that Cas protein.

In some embodiments, a catalytically inactive, e.g., dCas9, or partially deactivated Cas9 protein comprises a D11 mutation (e.g., D11A mutation) or an analogous substitution to the amino acid corresponding to said position. In some embodiments, a catalytically inactive Cas9 protein, e.g., dCas9, or partially deactivated Cas9 protein comprises a H969 mutation (e.g., H969A mutation) or an analogous substitution to the amino acid corresponding to said position. In some embodiments, a catalytically inactive Cas9 protein, e.g., dCas9, or partially deactivated Cas9 protein comprises a N995 mutation (e.g., N995A mutation) or an analogous substitution to the amino acid corresponding to said position. In some embodiments, a catalytically inactive Cas9 protein, e.g., dCas9, comprises mutations at one, two, or three of positions D11, H969, and N995 (e.g., D11A, H969A, and N995A mutations) or analogous substitutions to the amino acids corresponding to said positions.

In some embodiments, a catalytically inactive Cas9 protein, e.g., dCas9, or partially deactivated Cas9 protein comprises a D10 mutation (e.g., a D10A mutation) or an analogous substitution to the amino acid corresponding to said position. In some embodiments, a catalytically inactive Cas9 protein, e.g., dCas9, or partially deactivated Cas9 protein comprises a H557 mutation (e.g., a H557A mutation) or an analogous substitution to the amino acid corresponding to said position. In some embodiments, a catalytically inactive Cas9 protein, e.g., dCas9, comprises a D10 mutation (e.g., a D10A mutation) and a H557 mutation (e.g., a H557A mutation) or analogous substitutions to the amino acids corresponding to said positions.

In some embodiments, a catalytically inactive Cas9 protein, e.g., dCas9, or partially deactivated Cas9 protein comprises a D839 mutation (e.g., a D839A mutation) or an analogous substitution to the amino acid corresponding to said position. In some embodiments, a catalytically inactive Cas9 protein, e.g., dCas9, or partially deactivated Cas9 protein comprises a H840 mutation (e.g., a H840A mutation) or an analogous substitution to the amino acid corresponding to said position. In some embodiments, a catalytically inactive Cas9 protein, e.g., dCas9, or partially deactivated Cas9 protein comprises a N863 mutation (e.g., a N863A mutation) or an analogous substitution to the amino acid corresponding to said position. In some embodiments, a catalytically inactive Cas9 protein, e.g., dCas9, comprises a D10 mutation (e.g., D10A), a D839 mutation (e.g., D839A), a H840 mutation (e.g., H840A), and a N863 mutation (e.g., N863A) or analogous substitutions to the amino acids corresponding to said positions.

In some embodiments, a catalytically inactive Cas9 protein, e.g., dCas9, or partially deactivated Cas9 protein comprises an E993 mutation (e.g., a E993A mutation) or an analogous substitution to the amino acid corresponding to said position.

In some embodiments, a catalytically inactive Cas9 protein, e.g., dCas9, or partially deactivated Cas9 protein comprises a D917 mutation (e.g., a D917A mutation) or an analogous substitution to the amino acid corresponding to said position. In some embodiments, a catalytically inactive Cas9 protein, e.g., dCas9, or partially deactivated Cas9 protein comprises a a E1006 mutation (e.g., a E1006A mutation) or an analogous substitution to the amino acid corresponding to said position. In some embodiments, a catalytically inactive Cas9 protein, e.g., dCas9, or partially deactivated Cas9 protein comprises a D1255 mutation (e.g., a D1255A mutation) or an analogous substitution to the amino acid corresponding to said position. In some embodiments, a catalytically inactive Cas9 protein, e.g., dCas9, comprises a D917 mutation (e.g., D917A), a E1006 mutation (e.g., E1006A), and a D1255 mutation (e.g., D1255A) or analogous substitutions to the amino acids corresponding to said positions.

In some embodiments, a catalytically inactive Cas9 protein, e.g., dCas9, or partially deactivated Cas9 protein comprises a D16 mutation (e.g., a D16A mutation) or an analogous substitution to the amino acid corresponding to said position. In some embodiments, a catalytically inactive Cas9 protein, e.g., dCas9, or partially deactivated Cas9 protein comprises a D587 mutation (e.g., a D587A mutation) or an analogous substitution to the amino acid corresponding to said position. In some embodiments, a partially deactivated Cas domain has nickase activity. In some embodiments, a partially deactivated Cas9 domain is a Cas9 nickase domain. In some embodiments, the catalytically inactive Cas domain or dead Cas domain produces no detectable double strand break formation. In some embodiments, a catalytically inactive Cas9 protein, e.g., dCas9, or partially deactivated Cas9 protein comprises a H588 mutation (e.g., a H588A mutation) or an analogous substitution to the amino acid corresponding to said position. In some embodiments, a catalytically inactive Cas9 protein, e.g., dCas9, or partially deactivated Cas9 protein comprises a N611 mutation (e.g., a N611A mutation) or an analogous substitution to the amino acid corresponding to said position. In some embodiments, a catalytically inactive Cas9 protein, e.g., dCas9, comprises a D16 mutation (e.g., D16A), a D587 mutation (e.g., D587A), a H588 mutation (e.g., H588A), and a N611 mutation (e.g., N611A) or analogous substitutions to the amino acids corresponding to said positions.

In some embodiments, a DNA-binding domain or endonuclease domain may comprise a Cas molecule comprising or linked (e.g., covalently) to a gRNA (e.g., a template nucleic acid, e.g., template RNA, comprising a gRNA).

In some embodiments, an endonuclease domain or DNA binding domain comprises a Streptococcus pyogenes Cas9 (SpCas9) or a functional fragment or variant thereof. In some embodiments, the endonuclease domain or DNA binding domain comprises a modified SpCas9. In embodiments, the modified SpCas9 comprises a modification that alters protospacer-adjacent motif (PAM) specificity. In embodiments, the PAM has specificity for the nucleic acid sequence 5′-NGT-3′. In embodiments, the modified SpCas9 comprises one or more amino acid substitutions, e.g., at one or more of positions L1111, D1135, G1218, E1219, A1322, of R1335, e.g., selected from L1111R, D1135V, G1218R, E1219F, A1322R, R1335V. In embodiments, the modified SpCas9 comprises the amino acid substitution T1337R and one or more additional amino acid substitutions, e.g., selected from L1111, D1135L, S1136R, G1218S, E1219V, D1332A, D1332S, D1332T, D1332V, D1332L, D1332K, D1332R, R1335Q, T1337, T1337L, T1337Q, T1337I, T1337V, T1337F, T1337S, T1337N, T1337K, T1337H, T1337Q, and T1337M, or corresponding amino acid substitutions thereto. In embodiments, the modified SpCas9 comprises: (i) one or more amino acid substitutions selected from D1135L, S1136R, G1218S, E1219V, A1322R, R1335Q, and T1337; and (ii) one or more amino acid substitutions selected from L1111R, G1218R, E1219F, D1332A, D1332S, D1332T, D1332V, D1332L, D1332K, D1332R, T1337L, T1337I, T1337V, T1337F, T1337S, T1337N, T1337K, T1337R, T1337H, T1337Q, and T1337M, or corresponding amino acid substitutions thereto.

In some embodiments, the endonuclease domain or DNA binding domain comprises a Cas domain, e.g., a Cas9 domain. In embodiments, the endonuclease domain or DNA binding domain comprises a nuclease-active Cas domain, a Cas nickase (nCas) domain, or a nuclease-inactive Cas (dCas) domain. In embodiments, the endonuclease domain or DNA binding domain comprises a nuclease-active Cas9 domain, a Cas9 nickase (nCas9) domain, or a nuclease-inactive Cas9 (dCas9) domain. In some embodiments, the endonuclease domain or DNA binding domain comprises a Cas9 domain of Cas9 (e.g., dCas9 and nCas9), Cas12a/Cpf1, Cas12b/C2c1, Cas12c/C2c3, Cas12d/CasY, Cas12e/CasX, Cas12g, Cas12h, or Cas12i. In some embodiments, the endonuclease domain or DNA binding domain comprises a Cas9 (e.g., dCas9 and nCas9), Cas12a/Cpf1, Cas12b/C2c1, Cas12c/C2c3, Cas12d/CasY, Cas12e/CasX, Cas12g, Cas12h, or Cas12i. In some embodiments, the endonuclease domain or DNA binding domain comprises an S. pyogenes or an S. thermophilus Cas9, or a functional fragment thereof. In some embodiments, the endonuclease domain or DNA binding domain comprises a Cas9 sequence, e.g., as described in Chylinski, Rhun, and Charpentier (2013) RNA Biology 10:5, 726-737; incorporated herein by reference. In some embodiments, the endonuclease domain or DNA binding domain comprises the HNH nuclease subdomain and/or the RuvC1 subdomain of a Cas, e.g., Cas9, e.g., as described herein, or a variant thereof. In some embodiments, the endonuclease domain or DNA binding domain comprises Cas12a/Cpf1, Cas12b/C2c1, Cas12c/C2c3, Cas12d/CasY, Cas12e/CasX, Cas12g, Cas12h, or Cas12i. In some embodiments, the endonuclease domain or DNA binding domain comprises a Cas polypeptide (e.g., enzyme), or a functional fragment thereof. In embodiments, the Cas polypeptide (e.g., enzyme) is selected from Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (e.g., Csn1 or Csx12), Cas10, Cas10d, Cas12a/Cpf1, Cas12b/C2c1, Cas12c/C2c3, Cas12d/CasY, Cas12e/CasX, Cas12g, Cas12h, Cas12i, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, Cpf1, Cas12b/C2c1, Cas12c/C2c3, Cas12b/C2c1, Cas12c/C2c3, SpCas9(K855A), eSpCas9(1.1), SpCas9-HF1, hyper accurate Cas9 variant (HypaCas9), homologues thereof, modified or engineered versions thereof, and/or functional fragments thereof. In embodiments, the Cas9 comprises one or more substitutions, e.g., selected from H840A, D10A, P475A, W476A, N477A, D1125A, W1126A, and D1127A. In embodiments, the Cas9 comprises one or more mutations at positions selected from: D10, G12, G17, E762, H840, N854, N863, H982, H983, A984, D986, and/or A987, e.g., one or more substitutions selected from D10A, G12A, G17A, E762A, H840A, N854A, N863A, H982A, H983A, A984A, and/or D986A. In some embodiments, the endonuclease domain or DNA binding domain comprises a Cas (e.g., Cas9) sequence from Corynebacterium ulcerans, Corynebacterium diphtheria, Spiroplasma syrphidicola, Prevotella intermedia, Spiroplasma taiwanense, Streptococcus iniae, Belliella baltica, Psychroflexus torquis, Streptococcus thermophilus, Listeria innocua, Campylobacter jejuni, Neisseria meningitidis, Streptococcus pyogenes, or Staphylococcus aureus, or a fragment or variant thereof.

In some embodiments, the endonuclease domain or DNA binding domain comprises a Cpf1 domain, e.g., comprising one or more substitutions, e.g., at position D917, E1006A, D1255 or any combination thereof, e.g., selected from D917A, E1006A, D1255A, D917A/E1006A, D917A/D1255A, E1006A/D1255A, and D917A/E1006A/D1255A.

In some embodiments, the endonuclease domain or DNA binding domain comprises spCas9, spCas9-VRQR, spCas9-VRER, xCas9 (sp), saCas9, saCas9-KKH, spCas9-MQKSER, spCas9-LRKIQK, or spCas9-LRVSQL.

In some embodiments, a heterologous gene modifying polypeptide has an endonuclease domain comprising a Cas9 nickase, e.g., Cas9 H840A. In embodiments, the Cas9 H840A has the following amino acid sequence:

Cas9 nickase (H840A):
(SEQ ID NO: 11,001)
DKKYSIGLDIGTNSVGWAVITDEYKVPSKKFKVLGNTDRHSIKKNLIGALLFDSGETAEA
TRLKRTARRRYTRRKNRICYLQEIFSNEMAKVDDSFFHRLEESFLVEEDKKHERHPIFGN
IVDEVAYHEKYPTIYHLRKKLVDSTDKADLRLIYLALAHMIKFRGHFLIEGDLNPDNSDV
DKLFIQLVQTYNQLFEENPINASGVDAKAILSARLSKSRRLENLIAQLPGEKKNGLFGNLI
ALSLGLTPNFKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIFFDQSKNGYAG
YIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTEGMRKPAFLS
GEQKKAIVDLLFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQLKRRRYTGWG
RLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENQTTQKGQKNSRE
RMKRIEEGIKELGSQILKEHPVENTQLQNEKLYLYYLQNGRDMYVDQELDINRLSDYDV
DAIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVI
TLKSKLVSDFRKDFQFYKVREINNYHHAHDAYLNAVVGTALIKKYPKLESEFVYGDYK
VYDVRKMIAKSEQEIGKATAKYFFYSNIMNFFKTEITLANGEIRKRPLIETNGETGEIVWD
KGRDFATVRKVLSMPQVNIVKKTEVQTGGFSKESILPKRNSDKLIARKKDWDPKKYGG
FDSPTVAYSVLVVAKVEKGKSKKLKSVKELLGITIMERSSFEKNPIDFLEAKGYKEVKK
DLIIKLPKYSLFELENGRKRMLASAGELQKGNELALPSKYVNFLYLASHYEKLKGSPED
NEQKQLFVEQHKHYLDEIIEQISEFSKRVILADANLDKVLSAYNKHRDKPIREQAENIIHL
FTLTNLGAPAAFKYFDTTIDRKRYTSTKEVLDATLIHQSITGLYETRIDLSQLGGD

In some embodiments, a heterologous gene modifying polypeptide comprises a dCas9 sequence comprising a D10A and/or H840A mutation, e.g., the following sequence:

(SEQ ID NO: 5007)
SMDKKYSIGLAIGTNSVGWAVITDDYKVPSKKFKVLGNTDRHSIKKNLIGALLFDSGET
AEATRLKRTARRRYTRRKNRICYLQEIFSNEMAKVDDSFFHRLEESFLVEEDKKHERHPI
FGNIVDEVAYHEKYPTIYHLRKKLVDSTDKADLRLIYLALAHMIKFRGHFLIEGDLNPDN
SDVDKLFIQLVQTYNQLFEENPINASGVDAKAILSARLSKSRRLENLIAQLPGEKKNGLF
GNLIALSLGLTPNFKSNFDLAEDAKLQLSKDTYDDDLDNLLAQIGDQYADLFLAAKNLS
DAILLSDILRVNTEITKAPLSASMIKRYDEHHQDLTLLKALVRQQLPEKYKEIFFDQSKNG
YAGYIDGGASQEEFYKFIKPILEKMDGTEELLVKLNREDLLRKQRTFDNGSIPHQIHLGE
LHAILRRQEDFYPFLKDNREKIEKILTFRIPYYVGPLARGNSRFAWMTRKSEETITPWNFE
EVVDKGASAQSFIERMTNFDKNLPNEKVLPKHSLLYEYFTVYNELTKVKYVTEGMRKP
AFLSGEQKKAIVDLLFKTNRKVTVKQLKEDYFKKIECFDSVEISGVEDRFNASLGTYHDL
LKIIKDKDFLDNEENEDILEDIVLTLTLFEDREMIEERLKTYAHLFDDKVMKQLKRRRYT
GWGRLSRKLINGIRDKQSGKTILDFLKSDGFANRNFMQLIHDDSLTFKEDIQKAQVSGQ
GDSLHEHIANLAGSPAIKKGILQTVKVVDELVKVMGRHKPENIVIEMARENQTTQKGQK
NSRERMKRIEEGIKELGSQILKEHPVENTQLQNEKLYLYYLQNGRDMYVDQELDINRLS
DYDVDAIVPQSFLKDDSIDNKVLTRSDKNRGKSDNVPSEEVVKKMKNYWRQLLNAKLI
TQRKFDNLTKAERGGLSELDKAGFIKRQLVETRQITKHVAQILDSRMNTKYDENDKLIR
EVKVITLKSKLVSDFRKDFQFYKVREINNYHHAHDAYLNAVVGTALIKKYPKLESEFVY
GDYKVYDVRKMIAKSEQEIGKATAKYFFYSNIMNFFKTEITLANGEIRKRPLIETNGETG
EIVWDKGRDFATVRKVLSMPQVNIVKKTEVQTGGFSKESILPKRNSDKLIARKKDWDPK
KYGGFDSPTVAYSVLVVAKVEKGKSKKLKSVKELLGITIMERSSFEKNPIDFLEAKGYK
EVKKDLIIKLPKYSLFELENGRKRMLASAGELQKGNELALPSKYVNFLYLASHYEKLKG
SPEDNEQKQLFVEQHKHYLDEIIEQISEFSKRVILADANLDKVLSAYNKHRDKPIREQAE
NIIHLFTLTNLGAPAAFKYFDTTIDRKRYTSTKEVLDATLIHQSITGLYETRIDLSQLGGD

### TAL Effectors and Zinc Finger Nucleases

In some embodiments, an endonuclease domain or DNA-binding domain comprises a TAL effector molecule. A TAL effector molecule, e.g., a TAL effector molecule that specifically binds a DNA sequence, typically comprises a plurality of TAL effector domains or fragments thereof, and optionally one or more additional portions of naturally occurring TAL effectors (e.g., N- and/or C-terminal of the plurality of TAL effector domains). Many TAL effectors are known to those of skill in the art and are commercially available, e.g., from Thermo Fisher Scientific.

Naturally occurring TALEs are natural effector proteins secreted by numerous species of bacterial pathogens including the plant pathogen Xanthomonas which modulates gene expression in host plants and facilitates bacterial colonization and survival. The specific binding of TAL effectors is based on a central repeat domain of tandemly arranged nearly identical repeats of typically 33 or 34 amino acids (the repeat-variable di-residues, RVD domain).

Members of the TAL effectors family differ mainly in the number and order of their repeats. The number of repeats typically ranges from 1.5 to 33.5 repeats and the C-terminal repeat is usually shorter in length (e.g., about 20 amino acids) and is generally referred to as a “half-repeat.” Each repeat of the TAL effector generally features a one-repeat-to-one-base-pair correlation with different repeat types exhibiting different base-pair specificity (one repeat recognizes one base-pair on the target gene sequence). Generally, the smaller the number of repeats, the weaker the protein-DNA interactions. A number of 6.5 repeats has been shown to be sufficient to activate transcription of a reporter gene (Scholze et al., 2010).

Repeat to repeat variations occur predominantly at amino acid positions 12 and 13, which have therefore been termed “hypervariable” and which are responsible for the specificity of the interaction with the target DNA promoter sequence, as shown in Table 9 listing exemplary repeat variable diresidues (RVD) and their correspondence to nucleic acid base targets.

TABLE 9
RVDs and Nucleic Acid Base Specificity

| Target | Possible RVD Amino Acid Combinations | | | | | | | | | | | | |
|---|---|---|---|---|---|---|---|---|---|---|---|---|---|
| A | NI | NN | CI | HI | KI | | | | | | | | |
| G | NN | GN | SN | VN | LN | DN | QN | EN | HN | RH | NK | AN | FN |
| C | HD | RD | KD | ND | AD | | | | | | | | |
| T | NG | HG | VG | IG | EG | MG | YG | AA | EP | VA | QG | KG | RG |

Accordingly, it is possible to modify the repeats of a TAL effector to target specific DNA sequences. Further studies have shown that the RVD NK can target G. Target sites of TAL effectors also tend to include a T flanking the 5′ base targeted by the first repeat, but the exact mechanism of this recognition is not known. More than 113 TAL effector sequences are known to date. Non-limiting examples of TAL effectors from Xanthomonas include, Hax2, Hax3, Hax4, AvrXa7, AvrXa10 and AvrBs3.

Accordingly, the TAL effector domain of a TAL effector molecule described herein may be derived from a TAL effector from any bacterial species (e.g., Xanthomonas species such as the African strain of Xanthomonas oryzae pv. Oryzae (Yu et al. 2011), Xanthomonas campestris pv. raphani strain 756C and Xanthomonas oryzae pv. oryzicola strain BLS256 (Bogdanove et al. 2011). In some embodiments, the TAL effector domain comprises an RVD domain as well as flanking sequence(s) (sequences on the N-terminal and/or C-terminal side of the RVD domain) also from the naturally occurring TAL effector. It may comprise more or fewer repeats than the RVD of the naturally occurring TAL effector. The TAL effector molecule can be designed to target a given DNA sequence based on the above code and others known in the art. The number of TAL effector domains (e.g., repeats (monomers or modules)) and their specific sequence can be selected based on the desired DNA target sequence. For example, TAL effector domains, e.g., repeats, may be removed or added in order to suit a specific target sequence. In an embodiment, the TAL effector molecule of the present invention comprises between 6.5 and 33.5 TAL effector domains, e.g., repeats. In an embodiment, TAL effector molecule of the present invention comprises between 8 and 33.5 TAL effector domains, e.g., repeats, e.g., between 10 and 25 TAL effector domains, e.g., repeats, e.g., between 10 and 14 TAL effector domains, e.g., repeats.

In some embodiments, the TAL effector molecule comprises TAL effector domains that correspond to a perfect match to the DNA target sequence. In some embodiments, a mismatch between a repeat and a target base-pair on the DNA target sequence is permitted as along as it allows for the function of the polypeptide comprising the TAL effector molecule. In general, TALE binding is inversely correlated with the number of mismatches. In some embodiments, the TAL effector molecule of a polypeptide of the present invention comprises no more than 7 mismatches, 6 mismatches, 5 mismatches, 4 mismatches, 3 mismatches, 2 mismatches, or 1 mismatch, and optionally no mismatch, with the target DNA sequence. Without wishing to be bound by theory, in general the smaller the number of TAL effector domains in the TAL effector molecule, the smaller the number of mismatches will be tolerated and still allow for the function of the polypeptide comprising the TAL effector molecule. The binding affinity is thought to depend on the sum of matching repeat-DNA combinations. For example, TAL effector molecules having 25 TAL effector domains or more may be able to tolerate up to 7 mismatches.

In addition to the TAL effector domains, the TAL effector molecule of the present invention may comprise additional sequences derived from a naturally occurring TAL effector. The length of the C-terminal and/or N-terminal sequence(s) included on each side of the TAL effector domain portion of the TAL effector molecule can vary and be selected by one skilled in the art, for example based on the studies of Zhang et al. (2011). Zhang et al., have characterized a number of C-terminal and N-terminal truncation mutants in Hax3 derived TAL-effector based proteins and have identified key elements, which contribute to optimal binding to the target sequence and thus activation of transcription. Generally, it was found that transcriptional activity is inversely correlated with the length of N-terminus. Regarding the C-terminus, an important element for DNA binding residues within the first 68 amino acids of the Hax 3 sequence was identified. Accordingly, in some embodiments, the first 68 amino acids on the C-terminal side of the TAL effector domains of the naturally occurring TAL effector is included in the TAL effector molecule. Accordingly, in an embodiment, a TAL effector molecule comprises 1) one or more TAL effector domains derived from a naturally occurring TAL effector; 2) at least 70, 80, 90, 100, 110, 120, 130, 140, 150, 170, 180, 190, 200, 220, 230, 240, 250, 260, 270, 280 or more amino acids from the naturally occurring TAL effector on the N-terminal side of the TAL effector domains; and/or 3) at least 68, 80, 90, 100, 110, 120, 130, 140, 150, 170, 180, 190, 200, 220, 230, 240, 250, 260 or more amino acids from the naturally occurring TAL effector on the C-terminal side of the TAL effector domains.

In some embodiments, an endonuclease domain or DNA-binding domain is or comprises a Zn finger molecule. A Zn finger molecule comprises a Zn finger protein, e.g., a naturally occurring Zn finger protein or engineered Zn finger protein, or fragment thereof. Many Zn finger proteins are known to those of skill in the art and are commercially available, e.g., from Sigma-Aldrich.

In some embodiments, a Zn finger molecule comprises a non-naturally occurring Zn finger protein that is engineered to bind to a target DNA sequence of choice. See, for example, Beerli, et al. (2002) Nature Biotechnol. 20:135-141; Pabo, et al. (2001) Ann. Rev. Biochem. 70:313-340; Isalan, et al. (2001) Nature Biotechnol. 19:656-660; Segal, et al. (2001) Curr. Opin. Biotechnol. 12:632-637; Choo, et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

An engineered Zn finger protein may have a novel binding specificity, compared to a naturally-occurring Zn finger protein. Engineering methods include, but are not limited to, rational design and various types of selection. Rational design includes, for example, using databases comprising triplet (or quadruplet) nucleotide sequences and individual Zn finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as International Patent Publication Nos. WO 98/37186; WO 98/53057; WO 00/27878; and WO 01/88197 and GB 2,338,237. In addition, enhancement of binding specificity for zinc finger proteins has been described, for example, in International Patent Publication No. WO 02/077227.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein. In addition, enhancement of binding specificity for zinc finger binding domains has been described, for example, in co-owned International Patent Publication No. WO 02/077227.

Zn finger proteins and methods for design and construction of fusion proteins (and polynucleotides encoding same) are known to those of skill in the art and described in detail in U.S. Pat. Nos. 6,140,0815; 789,538; 6,453,242; 6,534,261; 5,925,523; 6,007,988; 6,013,453; and 6,200,759; International Patent Publication Nos. WO 95/19431; WO 96/06166; WO 98/53057; WO 98/54311; WO 00/27878; WO 01/60970; WO 01/88197; WO 02/099084; WO 98/53058; WO 98/53059; WO 98/53060; WO 02/016536; and WO 03/016496.

In addition, as disclosed in these and other references, Zn finger proteins and/or multi-fingered Zn finger proteins may be linked together, e.g., as a fusion protein, using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The Zn finger molecules described herein may include any combination of suitable linkers between the individual zinc finger proteins and/or multi-fingered Zn finger proteins of the Zn finger molecule.

In certain embodiments, the DNA-binding domain or endonuclease domain comprises a Zn finger molecule comprising an engineered zinc finger protein that binds (in a sequence-specific manner) to a target DNA sequence. In some embodiments, the Zn finger molecule comprises one Zn finger protein or fragment thereof. In other embodiments, the Zn finger molecule comprises a plurality of Zn finger proteins (or fragments thereof), e.g., 2, 3, 4, 5, 6 or more Zn finger proteins (and optionally no more than 12, 11, 10, 9, 8, 7, 6, 5, 4, 3, or 2 Zn finger proteins). In some embodiments, the Zn finger molecule comprises at least three Zn finger proteins. In some embodiments, the Zn finger molecule comprises four, five or six fingers. In some embodiments, the Zn finger molecule comprises 8, 9, 10, 11 or 12 fingers. In some embodiments, a Zn finger molecule comprising three Zn finger proteins recognizes a target DNA sequence comprising 9 or 10 nucleotides. In some embodiments, a Zn finger molecule comprising four Zn finger proteins recognizes a target DNA sequence comprising 12 to 14 nucleotides. In some embodiments, a Zn finger molecule comprising six Zn finger proteins recognizes a target DNA sequence comprising 18 to 21 nucleotides.

In some embodiments, a Zn finger molecule comprises a two-handed Zn finger protein. Two handed zinc finger proteins are those proteins in which two clusters of zinc finger proteins are separated by intervening amino acids so that the two zinc finger domains bind to two discontinuous target DNA sequences. An example of a two handed type of zinc finger binding protein is SIP1, where a cluster of four zinc finger proteins is located at the amino terminus of the protein and a cluster of three Zn finger proteins is located at the carboxyl terminus (see Remade, et al. (1999) EMBO Journal 18(18):5073-5084). Each cluster of zinc fingers in these proteins is able to bind to a unique target sequence and the spacing between the two target sequences can comprise many nucleotides.

### Exemplary Heterologous Gene Modifying Polypeptides

In some embodiments, a heterologous gene modifying polypeptide (e.g., a heterologous gene modifying polypeptide that is part of a system described herein) comprises an amino acid sequence as listed in Table T1, or an amino acid sequence having at least 70%, 75%, 80%, 85%, 90%, 95%, or 99% identity thereto. In some embodiments, a heterologous gene modifying polypeptide comprises a linker comprising a linker sequence as listed in Table T1, or an amino acid sequence having at least 70%, 75%, 80%, 85%, 90%, 95%, or 99% identity thereto. In some embodiments, a heterologous gene modifying polypeptide comprises an RT domain comprising an RT domain sequence as listed in Table T1, or an amino acid sequence having at least 70%, 75%, 80%, 85%, 90%, 95%, or 99% identity thereto. In some embodiments, a heterologous gene modifying polypeptide comprises: (i) a linker comprising a linker sequence as listed in a row of Table T1, or an amino acid sequence having at least 70%, 75%, 80%, 85%, 90%, 95%, or 99% identity thereto; and (ii) an RT domain comprising an RT domain sequence as listed in the same row of Table T1, or an amino acid sequence having at least 70%, 75%, 80%, 85%, 90%, 95%, or 99% identity thereto.

TABLE T1
Selection of exemplary heterologous gene modifying polypeptides

| SEQ ID NO: | | | |
|---|---|---|---|
| for Full | | SEQ ID | |
| Polypeptide | | NO: of | |
| Sequence | Linker Sequence | linker | RT name |
| 1372 | AEAAAKEAAAKEAAAKEAAAKALEAEAA | 15,401 | AVIRE_P03360_3mutA |
| | AKEAAAKEAAAKEAAAKA | | |
| 1197 | AEAAAKEAAAKEAAAKEAAAKALEAEAA | 15,402 | FLV_P10273_3mutA |
| | AKEAAAKEAAAKEAAAKA | | |
| 2784 | AEAAAKEAAAKEAAAKEAAAKALEAEAA | 15,403 | MLVMS_P03355_3mutA_ |
| | AKEAAAKEAAAKEAAAKA | | WS |
| 647 | AEAAAKEAAAKEAAAKEAAAKALEAEAA | 15,404 | SFV3L P27401_2mutA |
| | AKEAAAKEAAAKEAAAKA | | |

### Subsequences of Exemplary Heterologous Gene Modifying Polypeptides

In some embodiments, the heterologous gene modifying polypeptide comprises, in N-terminal to C-terminal order, one or more (e.g., 1, 2, 3, 4, 5, or all 6) of an N-terminal methionine residue, a first nuclear localization signal (NLS) (e.g., of a heterologous gene modifying polypeptide as listed in Table T1, or an amino acid sequence having at least 70%, 75%, 80%, 85%, 90%, 95%, or 99% identity thereto), a DNA binding domain (e.g., a Cas domain, e.g., a SpyCas9 domain, e.g., as listed in Table 8A, or an amino acid sequence having at least 70%, 75%, 80%, 85%, 90%, 95%, or 99% identity thereto; or a DNA binding domain of a heterologous gene modifying polypeptide as listed in Table T1, or an amino acid sequence having at least 70%, 75%, 80%, 85%, 90%, 95%, or 99% identity thereto), a linker (e.g., of a heterologous gene modifying polypeptide as listed in Table T1, or an amino acid sequence having at least 70%, 75%, 80%, 85%, 90%, 95%, or 99% identity thereto), an RT domain (e.g., of a heterologous gene modifying polypeptide as listed in Table T1, or an amino acid sequence having at least 70%, 75%, 80%, 85%, 90%, 95%, or 99% identity thereto), and a second NLS (e.g., of a heterologous gene modifying polypeptide as listed in Table T1, or an amino acid sequence having at least 70%, 75%, 80%, 85%, 90%, 95%, or 99% identity thereto). In some embodiments, the heterologous gene modifying polypeptide further comprises (e.g., C-terminal to the second NLS) a T2A sequence and/or a puromycin sequence (e.g., of a heterologous gene modifying polypeptide as listed in Table T1, or an amino acid sequence having at least 70%, 75%, 80%, 85%, 90%, 95%, or 99% identity thereto). In some embodiments, a nucleic acid encoding a heterologous gene modifying polypeptide (e.g., as described herein) encodes a T2A sequence, e.g., wherein the T2A sequence is situated between a region encoding the heterologous gene modifying polypeptide and a second region, wherein the second region optionally encodes a selectable marker, e.g., puromycin.

In certain embodiments, the first NLS comprises a first NLS sequence of a heterologous gene modifying polypeptide as listed in Table T1, or an amino acid sequence having at least 70%, 75%, 80%, 85%, 90%, 95%, or 99% identity thereto. In certain embodiments, the first NLS sequence comprises a C-myc NLS. In certain embodiments, the first NLS comprises the amino acid sequence PAAKRVKLD (SEQ ID NO: 11,095), or an amino acid sequence having at least 70%, 75%, 80%, 85%, 90%, 95%, or 99% identity thereto.

In certain embodiments, the heterologous gene modifying polypeptide further comprises a spacer sequence between the first NLS and the DNA binding domain. In certain embodiments, the spacer sequence between the first NLS and the DNA binding domain comprises 1, 2, 3, 4, 5, 6, 7, 8, 9, or 10 amino acids. In certain embodiments, the spacer sequence between the first NLS and the DNA binding domain comprises the amino acid sequence GG.

In certain embodiments, the DNA binding domain comprises a DNA binding domain of a heterologous gene modifying polypeptide as listed in Table T1, or an amino acid sequence having at least 70%, 75%, 80%, 85%, 90%, 95%, or 99% identity thereto. In certain embodiments, the DNA binding domain comprises a Cas domain (e.g., as listed in Table 8A). In certain embodiments, the DNA binding domain comprises the amino acid sequence of a SpyCas9 polypeptide (e.g., as listed in Table 8A, e.g., a Cas9 N863A polypeptide), or an amino acid sequence having at least 70%, 75%, 80%, 85%, 90%, 95%, or 99% identity thereto. In certain embodiments, the DNA binding domain comprises the amino acid sequence:

(SEQ ID NO: 11,096)
DKKYSIGLDIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDVDKLFIQLVQTYN
QLFEENPINASGVDAKAILSARLSKSRRLENLIAQLPGEKKNGLFGNLIALSLGLTPNFKSNFD
LAEDAKLQLSKDTYDDDLDNLLAQIGDQYADLFLAAKNLSDAILLSDILRVNTEITKAPLSASM
IKRYDEHHQDLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKILTFRIP
YYVGPLARGNSRFAWMTRKSEETITPWNFEEVVDKGASAQSFIERMTNFDKNLPNEKVLPKHSL
LYEYFTVYNELTKVKYVTEGMRKPAFLSGEQKKAIVDLLFKTNRKVTVKQLKEDYFKKIECFDS
VEISGVEDRFNASLGTYHDLLKIIKDKDFLDNEENEDILEDIVLTLTLFEDREMIEERLKTYAH
LFDDKVMKQLKRRRYTGWGRLSRKLINGIRDKQSGKTILDFLKSDGFANRNFMQLIHDDSLTFK
EDIQKAQVSGQGDSLHEHIANLAGSPAIKKGILQTVKVVDELVKVMGRHKPENIVIEMARENQT
TQKGQKNSRERMKRIEEGIKELGSQILKEHPVENTQLQNEKLYLYYLQNGRDMYVDQELDINRL
SDYDVDHIVPQSFLKDDSIDNKVLTRSDKARGKSDNVPSEEVVKKMKNYWRQLLNAKLITQRKF
DNLTKAERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVVAKVEKGK
SKKLKSVKELLGITIMERSSFEKNPIDFLEAKGYKEVKKDLIIKLPKYSLFELENGRKRMLASA
GELQKGNELALPSKYVNFLYLASHYEKLKGSPEDNEQKQLFVEQHKHYLDEIIEQISEFSKRVI
LADANLDKVLSAYNKHRDKPIREQAENIIHLFTLTNLGAPAAFKYFDTTIDRKRYTSTKEVLDA
TLIHQSITGLYETRIDLSQLGGD,

or an amino acid sequence having at least 70%, 75%, 80%, 85%, 90%, 95%, or 99% identity thereto.

In certain embodiments, the heterologous gene modifying polypeptide further comprises a spacer sequence between the DNA binding domain and the linker. In certain embodiments, the spacer sequence between the DNA binding domain and the linker comprises 1, 2, 3, 4, 5, 6, 7, 8, 9, or 10 amino acids. In certain embodiments, the spacer sequence between the DNA binding domain and the linker comprises the amino acid sequence GG.

In certain embodiments, the linker comprises a linker sequence of a heterologous gene modifying polypeptide as listed in Table T1, or an amino acid sequence having at least 70%, 75%, 80%, 85%, 90%, 95%, or 99% identity thereto.

In certain embodiments, the heterologous gene modifying polypeptide further comprises a spacer sequence between the linker and the RT domain. In certain embodiments, the spacer sequence between the linker and the RT domain comprises 1, 2, 3, 4, 5, 6, 7, 8, 9, or 10 amino acids. In certain embodiments, the spacer sequence between the linker and the RT domain comprises the amino acid sequence GG.

In certain embodiments, the RT domain comprises a RT domain sequence of a heterologous gene modifying polypeptide as listed in Table T1, or an amino acid sequence having at least 70%, 75%, 80%, 85%, 90%, 95%, or 99% identity thereto. In certain embodiments, the RT domain comprises an amino acid sequence as listed in Table 6, or an amino acid sequence having at least 70%, 75%, 80%, 85%, 90%, 95%, or 99% identity thereto. In some embodiments, the RT domain has a length of about 400-500, 500-600, 600-700, 700-800, 800-900, or 900-1000 amino acids.

In certain embodiments, the heterologous gene modifying polypeptide further comprises a spacer sequence between the RT domain and the second NLS. In certain embodiments, the spacer sequence between the RT domain and the second NLS comprises 1, 2, 3, 4, 5, 6, 7, 8, 9, or 10 amino acids. In certain embodiments, the spacer sequence between the RT domain and the second NLS comprises the amino acid sequence AG.

In certain embodiments, the second NLS comprises a second NLS sequence of a heterologous gene modifying polypeptide as listed in Table T1. In certain embodiments, the second NLS sequence comprises a plurality of partial NLS sequences. In embodiments, the NLS sequence, e.g., the second NLS sequence, comprises a first partial NLS sequence, e.g., comprising the amino acid sequence KRTADGSEFE (SEQ ID NO: 11,097), or an amino acid sequence having at least 70%, 75%, 80%, 85%, 90%, 95%, or 99% identity thereto. In embodiments, the NLS sequence, e.g., the second NLS sequence, comprises a second partial NLS sequence. In embodiments, the NLS sequence, e.g., the second NLS sequence, comprises an SV40A5 NLS, e.g., a bipartite SV40A5 NLS, e.g., comprising the amino acid sequence KRTADGSEFESPKKKAKVE (SEQ ID NO: 11,098), or an amino acid sequence having at least 70%, 75%, 80%, 85%, 90%, 95%, or 99% identity thereto. In certain embodiments, the NLS sequence, e.g., the second NLS sequence, comprises the amino acid sequence KRTADGSEFEKRTADGSEFESPKKKAKVE (SEQ ID NO: 11,099), or an amino acid sequence having at least 70%, 75%, 80%, 85%, 90%, 95%, or 99% identity thereto.

In certain embodiments, the heterologous gene modifying polypeptide further comprises a spacer sequence between the second NLS and the T2A sequence and/or puromycin sequence. In certain embodiments, the spacer sequence between the second NLS and the T2A sequence and/or puromycin sequence comprises 1, 2, 3, 4, 5, 6, 7, 8, 9, or 10 amino acids. In certain embodiments, the spacer sequence between the second NLS and the T2A sequence and/or puromycin sequence comprises the amino acid sequence GSG.

### Additional Linkers and RT Domains

In some embodiments, the heterologous gene modifying polypeptide comprises a linker (e.g., as described herein) and an RT domain (e.g., as described herein). In certain embodiments, the heterologous gene modifying polypeptide comprises, in N-terminal to C-terminal order, a linker (e.g., as described herein) and an RT domain (e.g., as described herein).

In certain embodiments, the linker comprises a linker sequence as listed in Table 10, or an amino acid sequence having at least 70%, 75%, 80%, 85%, 90%, 95%, or 99% identity thereto. In certain embodiments, the linker comprises a linker sequence of an exemplary heterologous gene modifying polypeptide listed in Table T1, or an amino acid sequence having at least 70%, 75%, 80%, 85%, 90%, 95%, or 99% identity thereto. In certain embodiments, the RT domain comprises an RT domain sequence as listed in Table 6, or an amino acid sequence having at least 70%, 75%, 80%, 85%, 90%, 95%, or 99% identity thereto. In certain embodiments, the RT domain comprises an RT domain sequence of an exemplary heterologous gene modifying polypeptide listed in Table T1, or an amino acid sequence having at least 70%, 75%, 80%, 85%, 90%, 95%, or 99% identity thereto.

In some embodiments, a heterologous gene modifying polypeptide comprises a linker of a gene modifying polypeptide as listed in Table T1, or a linker comprising an amino acid sequence having at least 70%, 75%, 80%, 85%, 90%, 95%, or 99% identity thereto.

In some embodiments, a heterologous gene modifying polypeptide comprises an RT domain of a heterologous gene modifying polypeptide as listed in Table T1, or an RT domain comprising an amino acid sequence having at least 70%, 75%, 80%, 85%, 90%, 95%, or 99% identity thereto.

In certain embodiments, the linker and the RT domain of a heterologous gene modifying polypeptide comprise the amino acid sequences of a linker and RT domain (or amino acid sequences having at least 70%, 75%, 80%, 85%, 90%, 95%, or 99% identity thereto) from a single row of Table T1 (e.g., from a single exemplary heterologous gene modifying polypeptide as listed in Table T1).

In certain embodiments, the linker and the RT domain of a heterologous gene modifying polypeptide comprise the amino acid sequences of a linker and RT domain (or amino acid sequences having at least 70%, 75%, 80%, 85%, 90%, 95%, or 99% identity thereto) from different rows of Table T1.

In certain embodiments, the heterologous gene modifying polypeptide further comprises a first NLS (e.g., a 5′ NLS), e.g., as described herein. In certain embodiments, the heterologous gene modifying polypeptide further comprises a second NLS (e.g., a 3′ NLS), e.g., as described herein. In certain embodiments, the heterologous gene modifying polypeptide further comprises an N-terminal methionine residue.

### RT Families and Mutants

In certain embodiments, the RT domain of a heterologous gene modifying polypeptide comprises the amino acid sequence of an RT domain of an AVIRE RT, or an amino acid sequence having at least 70%, 75%, 80%, 85%, 90%, 95%, or 99% identity thereto. In certain embodiments, the RT domain of a heterologous gene modifying polypeptide comprises the amino acid sequence of an RT domain of an MLVMS RT, or an amino acid sequence having at least 70%, 75%, 80%, 85%, 90%, 95%, or 99% identity thereto.

### Systems

In an aspect, the disclosure relates to a system comprising nucleic acid molecule encoding a heterologous gene modifying polypeptide (e.g., as described herein) and a template nucleic acid (e.g., a template RNA, e.g., as described herein). In certain embodiments, the nucleic acid molecule encoding the heterologous gene modifying polypeptide comprises one or more silent mutations in the coding region (e.g., in the sequence encoding the RT domain) relative to a nucleic acid molecule as described herein. In certain embodiments, the system further comprises a gRNA (e.g., a gRNA that binds to a polypeptide that induces a nick, e.g., in the opposite strand of the target DNA bound by the heterologous gene modifying polypeptide).

In certain embodiments, the nucleic acid molecule encoding the heterologous gene modifying polypeptide encodes a polypeptide as listed in Table T1, or an amino acid sequence having at least 70%, 75%, 80%, 85%, 90%, 95%, or 99% identity thereto.

In certain embodiments, the nucleic acid molecule encoding the heterologous gene modifying polypeptide comprises a sequence encoding a portion of a polypeptide listed in Table T1, wherein the portion comprises a linker and RT domain, or an amino acid sequence having at least 70%, 75%, 80%, 85%, 90%, 95%, or 99% identity to said portion.

In certain embodiments, the nucleic acid molecule encoding the heterologous gene modifying polypeptide comprises a sequence encoding the linker of a polypeptide as listed in Table T1, or an amino acid sequence having at least 70%, 75%, 80%, 85%, 90%, 95%, or 99% identity thereto.

In certain embodiments, the nucleic acid molecule encoding the heterologous gene modifying polypeptide comprises a sequence encoding the RT domain of a polypeptide as listed in Table T1, or an amino acid sequence having at least 70%, 75%, 80%, 85%, 90%, 95%, or 99% identity thereto.

In an aspect, the disclosure relates to a system comprising a heterologous gene modifying polypeptide (e.g., as described herein) and a template nucleic acid (e.g., a template RNA, e.g., as described herein).

In certain embodiments, the heterologous gene modifying polypeptide comprises a polypeptide as listed in Table T1, or an amino acid sequence having at least 70%, 75%, 80%, 85%, 90%, 95%, or 99% identity thereto.

In certain embodiments, the heterologous gene modifying polypeptide comprises a portion of a polypeptide listed in Table T1, wherein the portion comprises a linker and RT domain, or an amino acid sequence having at least 70%, 75%, 80%, 85%, 90%, 95%, or 99% identity to said portion.

In certain embodiments, the heterologous gene modifying polypeptide comprises the linker of a polypeptide as listed in Table T1, or an amino acid sequence having at least 70%, 75%, 80%, 85%, 90%, 95%, or 99% identity thereto.

In certain embodiments, the gene modifying polypeptide comprises the RT domain of a polypeptide as listed in Table T1, or an amino acid sequence having at least 70%, 75%, 80%, 85%, 90%, 95%, or 99% identity thereto.

### Additional Components of Heterologous Gene Modifying Systems

As disclosed herein, a heterologous gene modifying system RNA may further comprise an intracellular localization sequence. Additional details are provided in the section entitled “Localization sequence for gene modifying systems.” In some embodiments, the heterologous gene modifying system comprises an intein. Additional details are provided in the section entitled “Inteins.” In some embodiments, a heterologous gene modifying polypeptide may comprise a linker, e.g., a peptide linker, e.g., a linker as described in Table L1. Additional details are provided in the section entitled “Linkers”. The heterologous gene modifying polypeptide can bind a target DNA sequence and template nucleic acid (e.g., template RNA), nick the target site, and write (e.g., reverse transcribe) the template into DNA, resulting in a modification of the target site. In some embodiments, additional domains may be added to the polypeptide to enhance the efficiency of the process. In some embodiments, the heterologous gene modifying polypeptide may contain an additional DNA ligation domain to join reverse transcribed DNA to the DNA of the target site. In some embodiments, the polypeptide may comprise a heterologous RNA-binding domain. In some embodiments, the polypeptide may comprise a domain having 5′ to 3′ exonuclease activity (e.g., wherein the 5′ to 3′ exonuclease activity increases repair of the alteration of the target site, e.g., in favor of alteration over the original genomic sequence). In some embodiments, the polypeptide may comprise a domain having 3′ to 5′ exonuclease activity, e.g., proof-reading activity. In some embodiments, the writing domain, e.g., RT domain, has 3′ to 5′ exonuclease activity, e.g., proof-reading activity.

Template Nucleic Acids for Use with Heterologous Gene Modifying Polypeptides

The heterologous gene modifying systems described herein can modify a host target DNA site using a template nucleic acid sequence. In some embodiments, the systems described herein transcribe an RNA sequence template into host target DNA sites by target-primed reverse transcription (TPRT). By modifying DNA sequence(s) via reverse transcription of the RNA sequence template directly into the host genome, the system can insert an object sequence into a target genome without the need for exogenous DNA sequences to be introduced into the host cell (unlike, for example, CRISPR systems), as well as eliminate an exogenous DNA insertion step. The system can also delete a sequence from the target genome or introduce a substitution using an object sequence. Therefore, the system provides a platform for the use of customized RNA sequence templates containing object sequences, e.g., sequences comprising heterologous gene coding and/or function information.

In some embodiments, the template nucleic acid comprises one or more sequence (e.g., 2 sequences) that binds the heterologous gene modifying polypeptide.

In some embodiments a system or method described herein comprises a single template nucleic acid (e.g., template RNA). In some embodiments a system or method described herein comprises a plurality of template nucleic acids (e.g., template RNAs). For example, a system described herein comprises a first RNA comprising (e.g., from 5′ to 3′) a sequence that binds the heterologous gene modifying polypeptide (e.g., the DNA-binding domain and/or the endonuclease domain, e.g., a gRNA) and a sequence that binds a target site (e.g., a second strand of a site in a target genome), and a second RNA (e.g., a template RNA) comprising (e.g., from 5′ to 3′) optionally a sequence that binds the heterologous gene modifying polypeptide (e.g., that specifically binds the RT domain), a heterologous object sequence, and a PBS sequence. In some embodiments, when the system comprises a plurality of nucleic acids, each nucleic acid comprises a conjugating domain. In some embodiments, a conjugating domain enables association of nucleic acid molecules, e.g., by hybridization of complementary sequences. For example, in some embodiments a first RNA comprises a first conjugating domain and a second RNA comprises a second conjugating domain, and the first and second conjugating domains are capable of hybridizing to one another, e.g., under stringent conditions. In some embodiments, the stringent conditions for hybridization include hybridization in 4× sodium chloride/sodium citrate (SSC), at about 65 C, followed by a wash in 1×SSC, at about 65 C.

In some embodiments, the template nucleic acid comprises RNA. In some embodiments, the template nucleic acid comprises DNA (e.g., single stranded or double stranded DNA).

In some embodiments, the template nucleic acid comprises one or more (e.g., 2) homology domains that have homology to the target sequence. In some embodiments, the homology domains are about 10-20, 20-50, or 50-100 nucleotides in length.

In some embodiments, a template RNA can comprise a gRNA sequence, e.g., to direct the heterologous gene modifying polypeptide to a target site of interest. In some embodiments, a template RNA comprises (e.g., from 5′ to 3′) (i) optionally a gRNA spacer that binds a target site (e.g., a second strand of a site in a target genome), (ii) optionally a gRNA scaffold that binds a polypeptide described herein (e.g., a heterologous gene modifying polypeptide or a Cas polypeptide), (iii) a heterologous object sequence comprising a mutation region (optionally the heterologous object sequence comprises, from 5′ to 3′, a first homology region, a mutation region, and a second homology region), and (iv) a primer binding site (PBS) sequence comprising a 3′ target homology domain.

The template nucleic acid (e.g., template RNA) component of a genome editing system described herein typically is able to bind the heterologous gene modifying polypeptide of the system. In some embodiments the template nucleic acid (e.g., template RNA) has a 3′ region that is capable of binding a heterologous gene modifying polypeptide. The binding region, e.g., 3′ region, may be a structured RNA region, e.g., having at least 1, 2, or 3 hairpin loops, capable of binding the heterologous gene modifying polypeptide of the system. The binding region may associate the template nucleic acid (e.g., template RNA) with any of the polypeptide modules. In some embodiments, the binding region of the template nucleic acid (e.g., template RNA) may associate with an RNA-binding domain in the polypeptide. In some embodiments, the binding region of the template nucleic acid (e.g., template RNA) may associate with the reverse transcription domain of the heterologous gene modifying polypeptide (e.g., specifically bind to the RT domain). In some embodiments, the template nucleic acid (e.g., template RNA) may associate with the DNA binding domain of the polypeptide, e.g., a gRNA associating with a Cas9-derived DNA binding domain. In some embodiments, the binding region may also provide DNA target recognition, e.g., a gRNA hybridizing to the target DNA sequence and binding the polypeptide, e.g., a Cas9 domain. In some embodiments, the template nucleic acid (e.g., template RNA) may associate with multiple components of the polypeptide, e.g., DNA binding domain and reverse transcription domain.

In some embodiments the template RNA has a poly-A tail at the 3′ end. In some embodiments the template RNA does not have a poly-A tail at the 3′ end.

In some embodiments, the template nucleic acid is a template RNA. In some embodiments, the template RNA comprises one or more modified nucleotides. For example, in some embodiments, the template RNA comprises one or more deoxyribonucleotides. In some embodiments, regions of the template RNA are replaced by DNA nucleotides, e.g., to enhance stability of the molecule. For example, the 3′ end of the template may comprise DNA nucleotides, while the rest of the template comprises RNA nucleotides that can be reverse transcribed. For instance, in some embodiments, the heterologous object sequence is primarily or wholly made up of RNA nucleotides (e.g., at least 90%, 95%, 98%, or 99% RNA nucleotides). In some embodiments, the PBS sequence is primarily or wholly made up of DNA nucleotides (e.g., at least 90%, 95%, 98%, or 99% DNA nucleotides). In other embodiments, the heterologous object sequence for writing into the genome may comprise DNA nucleotides. In some embodiments, the DNA nucleotides in the template are copied into the genome by a domain capable of DNA-dependent DNA polymerase activity. In some embodiments, the DNA-dependent DNA polymerase activity is provided by a DNA polymerase domain in the polypeptide. In some embodiments, the DNA-dependent DNA polymerase activity is provided by a reverse transcriptase domain that is also capable of DNA-dependent DNA polymerization, e.g., second strand synthesis. In some embodiments, the template molecule is composed of only DNA nucleotides.

In some embodiments, a system described herein comprises two nucleic acids which together comprise the sequences of a template RNA described herein. In some embodiments, the two nucleic acids are associated with each other non-covalently, e.g., directly associated with each other (e.g., via base pairing), or indirectly associated as part of a complex comprising one or more additional molecule.

A template RNA described herein may comprise, from 5′ to 3′: (1) a gRNA spacer; (2) a gRNA scaffold; (3) heterologous object sequence (4) a primer binding site (PBS) sequence. Each of these components is now described in more detail.

gRNA Spacer and gRNA Scaffold

A template RNA described herein may comprise a gRNA spacer that directs the heterologous gene modifying system to a target nucleic acid, and a gRNA scaffold that promotes association of the template RNA with the Cas domain of the heterologous gene modifying polypeptide. The systems described herein can also comprise a gRNA that is not part of a template nucleic acid. For example, a gRNA that comprises a gRNA spacer and gRNA scaffold, but not a heterologous object sequence or a PBS sequence, can be used, e.g., to induce second strand nicking, e.g., as described in the section herein entitled “Second Strand Nicking”.

In some embodiments, the gRNA is a short synthetic RNA composed of a scaffold sequence that participates in CRISPR-associated protein binding and a user-defined ˜20 nucleotide targeting sequence for a genomic target. The structure of a complete gRNA was described by Nishimasu et al. Cell 156, P935-949 (2014). The gRNA (also referred to as sgRNA for single-guide RNA) consists of crRNA- and tracrRNA-derived sequences connected by an artificial tetraloop. The crRNA sequence can be divided into guide (20 nt) and repeat (12 nt) regions, whereas the tracrRNA sequence can be divided into anti-repeat (14 nt) and three tracrRNA stem loops (Nishimasu et al. Cell 156, P935-949 (2014)). In practice, guide RNA sequences are generally designed to have a length of between 17-24 nucleotides (e.g., 19, 20, or 21 nucleotides) and be complementary to a targeted nucleic acid sequence. Custom gRNA generators and algorithms are available commercially for use in the design of effective guide RNAs. In some embodiments, the gRNA comprises two RNA components from the native CRISPR system, e.g. crRNA and tracrRNA. As is well known in the art, the gRNA may also comprise a chimeric, single guide RNA (sgRNA) containing sequence from both a tracrRNA (for binding the nuclease) and at least one crRNA (to guide the nuclease to the sequence targeted for editing/binding). Chemically modified sgRNAs have also been demonstrated to be effective for use with CRISPR-associated proteins; see, for example, Hendel et al. (2015) Nature Biotechnol., 985-991. In some embodiments, a gRNA spacer comprises a nucleic acid sequence that is complementary to a DNA sequence associated with a target gene.

In some embodiments, the region of the template nucleic acid, e.g., template RNA, comprising the gRNA adopts an underwound ribbon-like structure of gRNA bound to target DNA (e.g., as described in Mulepati et al. Science 19 Sep. 2014:Vol. 345, Issue 6203, pp. 1479-1484). Without wishing to be bound by theory, this non-canonical structure is thought to be facilitated by rotation of every sixth nucleotide out of the RNA-DNA hybrid. Thus, in some embodiments, the region of the template nucleic acid, e.g., template RNA, comprising the gRNA may tolerate increased mismatching with the target site at some interval, e.g., every sixth base. In some embodiments, the region of the template nucleic acid, e.g., template RNA, comprising the gRNA comprising homology to the target site may possess wobble positions at a regular interval, e.g., every sixth base, that do not need to base pair with the target site.

In some embodiments, the template nucleic acid (e.g., template RNA) has at least 15, 16, 17, 18, 19, 20, 21, 22, 23, or 24 bases of at least 80%, 85%, 90%, 95%, 99%, or 100% homology to the target site, e.g., at the 5′ end, e.g., comprising a gRNA spacer sequence of length appropriate to the Cas9 domain of the heterologous gene modifying polypeptide (Table 8A).

In some embodiments, a Cas9 derivative with enhanced activity may be used in the gene modification polypeptide. In some embodiments, a Cas9 derivative may comprise mutations that improve activity of the HNH endonuclease domain, e.g., SpyCas9 R221K, N394K, or mutations that improve R-loop formation, e.g., SpyCas9 L1245V, or comprise a combination of such mutations, e.g., SpyCas9 R221K/N394K, SpyCas9 N394K/L1245V, SpyCas9 R221K/L1245V, or SpyCas9 R221K/N394K/L1245V (see, e.g., Spencer and Zhang Sci Rep 7:16836 (2017), the Cas9 derivatives and comprising mutations of which are incorporated herein by reference). In some embodiments, a Cas9 derivative may comprise one or more types of mutations described herein, e.g., PAM-modifying mutations, protein stabilizing mutations, activity enhancing mutations, and/or mutations partially or fully inactivating one or two endonuclease domains relative to the parental enzyme (e.g., one or more mutations to abolish endonuclease activity towards one or both strands of a target DNA, e.g., a nickase or catalytically dead enzyme). In some embodiments, a Cas9 enzyme used in a system described herein may comprise mutations that confer nickase activity toward the enzyme (e.g., SpyCas9 N863A or H840A) in addition to mutations improving catalytic efficiency (e.g., SpyCas9 R221K, N394K, and/or L1245V). In some embodiments, a Cas9 enzyme used in a system described herein is a SpyCas9 enzyme or derivative that further comprises an N863A mutation to confer nickase activity in addition to R221K and N394K mutations to improve catalytic efficiency.

Table 12 provides parameters to define components for designing gRNA and/or Template RNAs to apply Cas variants listed in Table 8A for gene modifying. The cut site indicates the validated or predicted protospacer adjacent motif (PAM) requirements, validated or predicted location of cut site (relative to the most upstream base of the PAM site). The gRNA for a given enzyme can be assembled by concatenating the crRNA, Tetraloop, and tracrRNA sequences, and further adding a 5′ spacer of a length within Spacer (min) and Spacer (max) that matches a protospacer at a target site. Further, the predicted location of the ssDNA nick at the target is important for designing a PBS sequence of a Template RNA that can anneal to the sequence immediately 5′ of the nick in order to initiate target primed reverse transcription. In some embodiments, a gRNA scaffold described herein comprises a nucleic acid sequence comprising, in the 5′ to 3′ direction, a crRNA of Table 12, a tetraloop from the same row of Table 12, and a tracrRNA from the same row of Table 12, or a sequence having at least 70%, 80%, 85%, 90%, 95%, or 99% identity thereto. In some embodiments, the gRNA or template RNA comprising the scaffold further comprises a gRNA spacer having a length within the Spacer (min) and Spacer (max) indicated in the same row of Table 12. In some embodiments, the gRNA or template RNA having a sequence according to Table 12 is comprised by a system that further comprises a heterologous gene modifying polypeptide, wherein the heterologous gene modifying polypeptide comprises a Cas domain described in the same row of Table 12.

TABLE 12
Parameters to define components for designing gRNA and/or Template RNAs to apply Cas variants listed in Table 8A
in systems

| | | | | Spacer | Spacer | | SEQ ID | | | SEQ ID |
|---|---|---|---|---|---|---|---|---|---|---|
| Variant | PAM(s) | Cut | Tier | (min) | (max) | crRNA | NO: | Tetraloop | tracrRNA | NO: |
| Nme2Cas9 | NNNNCC | −3 | 1 | 22 | 24 | GTTGTAG | 10,051 | GAAA | CGAAATGAGAACCGTTGCTACAAT | 10,151 |
| | | | | | | CTCCCTT | | | AAGGCCGTCTGAAAAGATGTGCCG | |
| | | | | | | TCTCATT | | | CAACGCTCTGCCCCTTAAAGCTTC | |
| | | | | | | TCG | | | TGCTTTAAGGGGCATCGTTTA | |
| PpnCas9 | NNNNRT | | 1 | 21 | 24 | GTTGTAG | 10,052 | GAAA | GCGAAATGAAAAACGTTGTTACAA | 10,152 |
| | T | | | | | CTCCCTT | | | TAAGAGATGAATTTCTCGCAAAGC | |
| | | | | | | TTTCATT | | | TCTGCCTCTTGAAATTTCGGTTTCA | |
| | | | | | | TCGC | | | AGAGGCATC | |
| SauCas9 | NNGRR; | −3 | 1 | 21 | 23 | GTTTTAG | 10,053 | GAAA | CAGAATCTACTAAAACAAGGCAA | 10,153 |
| | NNGRRT | | | | | TACTCTG | | | AATGCCGTGTTTATCTCGTCAACTT | |
| | | | | | | | | | GTTGGCGAGA | |
| SauCas9- | NNNRR; | −3 | 1 | 21 | 21 | GTTTTAG | 10,054 | GAAA | CAGAATCTACTAAAACAAGGCAA | 10,154 |
| KKH | NNNRRT | | | | | TACTCTG | | | AATGCCGTGTTTATCTCGTCAACTT | |
| | | | | | | | | | GTTGGCGAGA | |
| SauriCas9 | NNGG | −3 | 1 | 21 | 21 | GTTTTAG | 10,055 | GAAA | CAGAATCTACTAAAACAAGGCAA | 10,155 |
| | | | | | | TACTCTG | | | AATGCCGTGTTTATCTCGTCAACTT | |
| | | | | | | | | | GTTGGCGAGA | |
| SauriCas9- | NNRG | −3 | 1 | 21 | 21 | GTTTTAG | 10,056 | GAAA | CAGAATCTACTAAAACAAGGCAA | 10,156 |
| KKH | | | | | | TACTCTG | | | AATGCCGTGTTTATCTCGTCAACTT | |
| | | | | | | | | | GTTGGCGAGA | |
| ScaCas9-Sc++ | NNG | −3 | 1 | 20 | 20 | GTTTTAG | 10,057 | GAAA | TAGCAAGTTAAAATAAGGCTAGTC | 10,157 |
| | | | | | | AGCTA | | | CGTTATCAACTTGAAAAAGTGGCA | |
| | | | | | | | | | CCGAGTCGGTGC | |
| Spy Cas9 | NGG | −3 | 1 | 20 | 20 | GTTTTAG | 10,058 | GAAA | TAGCAAGTTAAAATAAGGCTAGTC | 10,158 |
| | | | | | | AGCTA | | | CGTTATCAACTTGAAAAAGTGGCA | |
| | | | | | | | | | CCGAGTCGGTGC | |
| SpyCas9_i_v1 | NGG | −3 | 1 | 20 | 20 | GTTTTAG | 10,058 | GAAA | TAGCAAGTTAAAATAAGGCTAGTC | 10,193 |
| | | | | | | AGCTA | | | CGTTATCAACTTGGACTTCGGTCC | |
| | | | | | | | | | AAGTGGCACCGAGTCGGTGC | |
| SpyCas9_i_v2 | NGG | −3 | 1 | 20 | 20 | GTTTTAG | 10,058 | GAAA | TAGCAAGTTAAAATAAGGCTAGTC | 10,194 |
| | | | | | | AGCTA | | | CGTTATCAACTTGGAGCTTGCTCC | |
| | | | | | | | | | AAGTGGCACCGAGTCGGTGC | |
| SpyCas9_i_v3 | NGG | −3 | 1 | 20 | 20 | GTTTTAG | 10,058 | GAAA | GTTTTAGAGCTAGAAATAGCAAGT | 10,195 |
| | | | | | | AGCTA | | | TAAAATAAGGCTAGTCCGTTATCG | |
| | | | | | | | | | ACTTGAAAAAGTCGCACCGAGTCG | |
| | | | | | | | | | GTGC | |
| Spy Cas9-NG | NG | −3 | 1 | 20 | 20 | GTTTTAG | 10,059 | GAAA | TAGCAAGTTAAAATAAGGCTAGTC | 10,159 |
| | (NGG = NGA = | | | | | AGCTA | | | CGTTATCAACTTGAAAAAGTGGCA | |
| | NGT > NGC) | | | | | | | | CCGAGTCGGTGC | |
| Spy Cas9- | NRN > NYN | −3 | 1 | 20 | 20 | GTTTTAG | 10,060 | GAAA | TAGCAAGTTAAAATAAGGCTAGTC | 10,160 |
| SpRY | | | | | | AGCTA | | | CGTTATCAACTTGAAAAAGTGGCA | |
| | | | | | | | | | CCGAGTCGGTGC | |
| St1Cas9 | NNAGAAW > | −3 | 1 | 20 | 20 | GTCTTTG | 10,061 | GTAC | CAGAAGCTACAAAGATAAGGCTTC | 10,161 |
| | NNAGGAW = | | | | | TACTCTG | | | ATGCCGAAATCAACACCCTGTCAT | |
| | NNGGAAW | | | | | | | | TTTATGGCAGGGTGTTTT | |
| BlatCas9 | NNNNCNAA > | −3 | 1 | 19 | 23 | GCTATAG | 10,062 | GAAA | GGTAAGTTGCTATAGTAAGGGCAA | 10,162 |
| | NNNNCNDD > | | | | | TTCCTTA | | | CAGACCCGAGGCGTTGGGGATCGC | |
| | NNNNC | | | | | CT | | | CTAGCCCGTGTTTACGGGCTCTCC | |
| | | | | | | | | | CCATATTCAAAATAATGACAGACG | |
| | | | | | | | | | AGCACCTTGGAGCATTTATCTCCG | |
| | | | | | | | | | AGGTGCT | |
| cCas9-v16 | NNVACT; | −3 | 2 | 21 | 21 | GTCTTAG | 10,063 | GAAA | CAGAATCTACTAAGACAAGGCAA | 10,163 |
| | NNVATGM; | | | | | TACTCTG | | | AATGCCGTGTTTATCTCGTCAACTT | |
| | NNVATT; | | | | | | | | GTTGGCGAGA | |
| | NNVGCT; | | | | | | | | | |
| | NNVGTG; | | | | | | | | | |
| | NNVGTT | | | | | | | | | |
| cCas9-v17 | NNVRRN | −3 | 2 | 21 | 21 | GTCTTAG | 10,064 | GAAA | CAGAATCTACTAAGACAAGGCAA | 10,164 |
| | | | | | | TACTCTG | | | AATGCCGTGTTTATCTCGTCAACTT | |
| | | | | | | | | | GTTGGCGAGA | |
| cCas9-v21 | NNVACT; | −3 | 2 | 21 | 21 | GTCTTAG | 10,065 | GAAA | CAGAATCTACTAAGACAAGGCAA | 10,165 |
| | NNVATGM; | | | | | TACTCTG | | | AATGCCGTGTTTATCTCGTCAACTT | |
| | NNVATT; | | | | | | | | GTTGGCGAGA | |
| | NNVGCT; | | | | | | | | | |
| | NNVGTG; | | | | | | | | | |
| | NNVGTT | | | | | | | | | |
| cCas9-v42 | NNVRRN | −3 | 2 | 21 | 21 | GTCTTAG | 10,066 | GAAA | CAGAATCTACTAAGACAAGGCAA | 10,166 |
| | | | | | | TACTCTG | | | AATGCCGTGTTTATCTCGTCAACTT | |
| | | | | | | | | | GTTGGCGAGA | |
| CdiCas9 | NNRHHHY; | | 2 | 22 | 22 | ACTGGG | 10,067 | GAAA | CTGAACCTCAGTAAGCATTGGCTC | 10,167 |
| | NNRAAAY | | | | | GTTCAG | | | GTTTCCAATGTTGATTGCTCCGCC | |
| | | | | | | | | | GGTGCTCCTTATTTTTAAGGGCGC | |
| | | | | | | | | | CGGC | |
| CjeCas9 | NNNNR | −3 | 2 | 21 | 23 | GTTTTAG | 10,068 | GAAA | AGGGACTAAAATAAAGAGTTTGC | 10,168 |
| | YAC | | | | | TCCCT | | | GGGACTCTGCGGGGTTACAATCCC | |
| | | | | | | | | | CTAAAACCGC | |
| GeoCas9 | NNNNCR | | 2 | 21 | 23 | GTCATAG | 10,069 | GAAA | TCAGGGTTACTATGATAAGGGCTT | 10,169 |
| | AA | | | | | TTCCCCT | | | TCTGCCTAAGGCAGACTGACCCGC | |
| | | | | | | GA | | | GGCGTTGGGGATCGCCTGTCGCCC | |
| | | | | | | | | | GCTTTTGGCGGGCATTCCCCATCC | |
| | | | | | | | | | TT | |
| iSpy MacCas9 | NAAN | −3 | 2 | 19 | 21 | GTTTTAG | 10,070 | GAAA | TAGCAAGTTAAAATAAGGCTAGTC | 10,170 |
| | | | | | | AGCTA | | | CGTTATCAACTTGAAAAAGTGGCA | |
| | | | | | | | | | CCGAGTCGGTGC | |
| NmeCas9 | NNNNGAYT; | −3 | 2 | 20 | 24 | GTTGTAG | 10,071 | GAAA | CGAAATGAGAACCGTTGCTACAAT | 10,171 |
| | NNNNGYTT; | | | | | CTCCCTT | | | AAGGCCGTCTGAAAAGATGTGCCG | |
| | NNNNGAYA; | | | | | TCTCATT | | | CAACGCTCTGCCCCTTAAAGCTTC | |
| | NNNNGTCT | | | | | TCG | | | TGCTTTAAGGGGCATCGTTTA | |
| ScaCas9 | NNG | −3 | 2 | 20 | 20 | GTTTTAG | 10,072 | GAAA | TAGCAAGTTAAAATAAGGCTAGTC | 10,172 |
| | | | | | | AGCTA | | | CGTTATCAACTTGAAAAAGTGGCA | |
| | | | | | | | | | CCGAGTCGGTGC | |
| ScaCas9- | NNG | −3 | 2 | 20 | 20 | GTTTTAG | 10,073 | GAAA | TAGCAAGTTAAAATAAGGCTAGTC | 10,173 |
| HiFi-Sc++ | | | | | | AGCTA | | | CGTTATCAACTTGAAAAAGTGGCA | |
| | | | | | | | | | CCGAGTCGGTGC | |
| Spy Cas9- | NRRH | −3 | 2 | 20 | 20 | GTTTAAG | 10,074 | GAAA | CAGCATAGCAAGTTTAAATAAGGC | 10,174 |
| 3var-NRRH | | | | | | AGCTATG | | | TAGTCCGTTATCAACTTGAAAAAG | |
| | | | | | | CTG | | | TGGCACCGAGTCGGTGC | |
| Spy Cas9- | NRTH | −3 | 2 | 20 | 20 | GTTTAAG | 10,075 | GAAA | CAGCATAGCAAGTTTAAATAAGGC | 10,175 |
| 3var-NRTH | | | | | | AGCTATG | | | TAGTCCGTTATCAACTTGAAAAAG | |
| | | | | | | CTG | | | TGGCACCGAGTCGGTGC | |
| Spy Cas9- | NRCH | −3 | 2 | 20 | 20 | GTTTAAG | 10,076 | GAAA | CAGCATAGCAAGTTTAAATAAGGC | 10,176 |
| 3var-NRCH | | | | | | AGCTATG | | | TAGTCCGTTATCAACTTGAAAAAG | |
| | | | | | | CTG | | | TGGCACCGAGTCGGTGC | |
| Spy Cas9-HF1 | NGG | −3 | 2 | 20 | 20 | GTTTTAG | 10,077 | GAAA | TAGCAAGTTAAAATAAGGCTAGTC | 10,177 |
| | | | | | | AGCTA | | | CGTTATCAACTTGAAAAAGTGGCA | |
| | | | | | | | | | CCGAGTCGGTGC | |
| Spy Cas9- | NAAG | −3 | 2 | 20 | 20 | GTTTTAG | 10,078 | GAAA | TAGCAAGTTAAAATAAGGCTAGTC | 10,178 |
| QQR1 | | | | | | AGCTA | | | CGTTATCAACTTGAAAAAGTGGCA | |
| | | | | | | | | | CCGAGTCGGTGC | |
| Spy Cas9-SpG | NGN | −3 | 2 | 20 | 20 | GTTTTAG | 10,079 | GAAA | TAGCAAGTTAAAATAAGGCTAGTC | 10,179 |
| | | | | | | AGCTA | | | CGTTATCAACTTGAAAAAGTGGCA | |
| | | | | | | | | | CCGAGTCGGTGC | |
| Spy Cas9- | NGAN | −3 | 2 | 20 | 20 | GTTTTAG | 10,080 | GAAA | TAGCAAGTTAAAATAAGGCTAGTC | 10,180 |
| VQR | | | | | | AGCTA | | | CGTTATCAACTTGAAAAAGTGGCA | |
| | | | | | | | | | CCGAGTCGGTGC | |
| Spy Cas9- | NGCG | −3 | 2 | 20 | 20 | GTTTTAG | 10,081 | GAAA | TAGCAAGTTAAAATAAGGCTAGTC | 10,181 |
| VRER | | | | | | AGCTA | | | CGTTATCAACTTGAAAAAGTGGCA | |
| | | | | | | | | | CCGAGTCGGTGC | |
| Spy Cas9-xCas | NG; GAA; | −3 | 2 | 20 | 20 | GTTTAAG | 10,082 | GAAA | CAGCATAGCAAGTTTAAATAAGGC | 10,182 |
| | GAT | | | | | AGCTATG | | | TAGTCCGTTATCAACTTGAAAAAG | |
| | | | | | | CTG | | | TGGCACCGAGTCGGTGC | |
| Spy Cas9- | NG | −3 | 2 | 20 | 20 | GTTTAAG | 10,083 | GAAA | CAGCATAGCAAGTTTAAATAAGGC | 10,183 |
| xCas-NG | | | | | | AGCTATG | | | TAGTCCGTTATCAACTTGAAAAAG | |
| | | | | | | CTG | | | TGGCACCGAGTCGGTGC | |
| St1Cas9- | NNACA | −3 | 2 | 20 | 20 | GTCTTTG | 10,084 | GTAC | CAGAAGCTACAAAGATAAGGCTTC | 10,184 |
| CNRZ1066 | A | | | | | TACTCTG | | | ATGCCGAAATCAACACCCTGTCAT | |
| | | | | | | | | | TTTATGGCAGGGTGTTTT | |
| St1Cas9- | NNGCA | −3 | 2 | 20 | 20 | GTCTTTG | 10,085 | GTAC | CAGAAGCTACAAAGATAAGGCTTC | 10,185 |
| LMG1831 | A | | | | | TACTCTG | | | ATGCCGAAATCAACACCCTGTCAT | |
| | | | | | | | | | TTTATGGCAGGGTGTTTT | |
| St1Cas9- | NNAAA | −3 | 2 | 20 | 20 | GTCTTTG | 10,086 | GTAC | CAGAAGCTACAAAGATAAGGCTTC | 10,186 |
| MTH17CL396 | A | | | | | TACTCTG | | | ATGCCGAAATCAACACCCTGTCAT | |
| | | | | | | | | | TTTATGGCAGGGTGTTTT | |
| St1Cas9- | NNGAA | −3 | 2 | 20 | 20 | GTCTTTG | 10,087 | GTAC | CAGAAGCTACAAAGATAAGGCTTC | 10,187 |
| TH1477 | A | | | | | TACTCTG | | | ATGCCGAAATCAACACCCTGTCAT | |
| | | | | | | | | | TTTATGGCAGGGTGTTTT | |
| sRGN3.1 | NNGG | | 1 | 21 | 23 | GTTTTAG | 10,088 | GAAA | CAGAATCTACTGAAACAAGACAAT | 10,188 |
| | | | | | | TACTCTG | | | ATGTCGTGTTTATCCCATCAATTTA | |
| | | | | | | | | | TTGGTGGGATTTT | |
| sRGN3.3 | NNGG | | 1 | 21 | 23 | GTTTTAG | 10,089 | GAAA | CAGAATCTACTGAAACAAGACAAT | 10,189 |
| | | | | | | TACTCTG | | | ATGTCGTGTTTATCCCATCAATTTA | |
| | | | | | | | | | TTGGTGGGATTTT | |

Herein, when an RNA sequence (e.g., a template RNA sequence) is said to comprise a particular sequence (e.g., a sequence of Table 12 or a portion thereof) that comprises thymine (T), it is of course understood that the RNA sequence may (and frequently does) comprise uracil (U) in place of T. For instance, the RNA sequence may comprise U at every position shown as T in the sequence in Table 12. More specifically, the present disclosure provides an RNA sequence according to every gRNA scaffold sequence of Table 12, wherein the RNA sequence has a U in place of each T in the sequence in Table 12. Additionally, it is understood that terminal Us and Ts may optionally be added or removed from tracrRNA sequences and may be modified or unmodified when provided as RNA. Without wishing to be bound by example, versions of gRNA scaffold sequences alternative to those exemplified in Table 12 may also function with the different Cas9 enzymes or derivatives thereof exemplified in Table 8A, e.g., alternate gRNA scaffold sequences with nucleotide additions, substitutions, or deletions, e.g., sequences with stem-loop structures added or removed. It is contemplated herein that the gRNA scaffold sequences represent a component of systems that can be similarly optimized for a given system, Cas-RT fusion polypeptide, indication, target mutation, template RNA, or delivery vehicle.

### Heterologous Object Sequence

A template RNA described herein may comprise a heterologous object sequence that the heterologous gene modifying polypeptide can use as a template for reverse transcription, to write a desired sequence into the target nucleic acid. In some embodiments, the heterologous object sequence comprises, from 5′ to 3′, a post-edit homology region, the mutation region, and a pre-edit homology region. Without wishing to be bound by theory, an RT performing reverse transcription on the template RNA first reverse transcribes the pre-edit homology region, then the mutation region, and then the post-edit homology region, thereby creating a DNA strand comprising the desired mutation with a homology region on either side.

In some embodiments, the heterologous object sequence is at least 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 120, 140, 160, 180, 200, 500, or 1,000 nucleotides (nts) in length, or at least 1, 1.5, 2, 2.5, 3, 3.5, 4, 4.5, 5, 5.5, 6, 6.5, 7, 7.5, 8, 8.5, 9, 9.5, or 10 kilobases in length. In some embodiments, the heterologous object sequence is no more than 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 120, 140, 160, 180, 200, 500, 1,000, or 2000 nucleotides (nts) in length, or no more than 20, 15, 10, 9, 8, 7, 6, 5, 4, or 3 kilobases in length. In some embodiments, the heterologous object sequence is 30-1000, 40-1000, 50-1000, 60-1000, 70-1000, 74-1000, 75-1000, 76-1000, 77-1000, 78-1000, 79-1000, 80-1000, 85-1000, 90-1000, 100-1000, 120-1000, 140-1000, 160-1000, 180-1000, 200-1000, 500-1000, 30-500, 40-500, 50-500, 60-500, 70-500, 74-500, 75-500, 76-500, 77-500, 78-500, 79-500, 80-500, 85-500, 90-500, 100-500, 120-500, 140-500, 160-500, 180-500, 200-500, 30-200, 40-200, 50-200, 60-200, 70-200, 74-200, 75-200, 76-200, 77-200, 78-200, 79-200, 80-200, 85-200, 90-200, 100-200, 120-200, 140-200, 160-200, 180-200, 30-100, 40-100, 50-100, 60-100, 70-100, 74-100, 75-100, 76-100, 77-100, 78-100, 79-100, 80-100, 85-100, or 90-100 nucleotides (nts) in length, or 1-20, 1-15, 1-10, 1-9, 1-8, 1-7, 1-6, 1-5, 1-4, 1-3, 1-2, 2-20, 2-15, 2-10, 2-9, 2-8, 2-7, 2-6, 2-5, 2-4, 2-3, 3-20, 3-15, 3-10, 3-9, 3-8, 3-7, 3-6, 3-5, 3-4, 4-20, 4-15, 4-10, 4-9, 4-8, 4-7, 4-6, 4-5, 5-20, 5-15, 5-10, 5-9, 5-8, 5-7, 5-6, 6-20, 6-15, 6-10, 6-9, 6-8, 6-7, 7-20, 7-15, 7-10, 7-9, 7-8, 8-20, 8-15, 8-10, 8-9, 9-20, 9-15, 9-10, 10-15, 10-20, or 15-20 kilobases in length. In some embodiments, the heterologous object sequence is 10-100, 10-90, 10-80, 10-70, 10-60, 10-50, 10-40, 10-30, or 10-20 nt in length, e.g., 10-80, 10-50, or 10-20 nt in length, e.g., about 10-20 nt in length. In some embodiments, the heterologous object sequence is 8-30, 9-25, 10-20, 11-16, or 12-15 nucleotides in length, e.g., is 11-16 nt in length. Without wishing to be bound by theory, in some embodiments, a larger insertion size, larger region of editing (e.g., the distance between a first edit/substitution and a second edit/substitution in the target region), and/or greater number of desired edits (e.g., mismatches of the heterologous object sequence to the target genome), may result in a longer optimal heterologous object sequence.

In certain embodiments, the template nucleic acid comprises a customized RNA sequence template which can be identified, designed, engineered and constructed to contain sequences altering or specifying host genome function, for example by introducing a heterologous coding region into a genome; affecting or causing exon structure/alternative splicing, e.g., leading to exon skipping of one or more exons; causing disruption of an endogenous gene, e.g., creating a genetic knockout; causing transcriptional activation of an endogenous gene; causing epigenetic regulation of an endogenous DNA; causing up-regulation of one or more operably linked genes, e.g., leading to gene activation or overexpression; causing down-regulation of one or more operably linked genes, e.g., creating a genetic knock-down; etc. In certain embodiments, a customized RNA sequence template can be engineered to contain sequences coding for exons and/or transgenes, provide binding sites for transcription factor activators, repressors, enhancers, etc., and combinations thereof. In some embodiments, a customized template can be engineered to encode a nucleic acid or peptide tag to be expressed in an endogenous RNA transcript or endogenous protein operably linked to the target site. In other embodiments, the coding sequence can be further customized with splice donor sites, splice acceptor sites, or poly-A tails.

The template nucleic acid (e.g., template RNA) of the system typically comprises an object sequence (e.g., a heterologous object sequence) for writing a desired sequence into a target DNA. The object sequence may be coding or non-coding. The template nucleic acid (e.g., template RNA) can be designed to result in insertions, mutations, or deletions at the target DNA locus. In some embodiments, the template nucleic acid (e.g., template RNA) may be designed to cause an insertion in the target DNA. For example, the template nucleic acid (e.g., template RNA) may contain a heterologous sequence, wherein the reverse transcription will result in insertion of the heterologous sequence into the target DNA. In other embodiments, the RNA template may be designed to introduce a deletion into the target DNA. For example, the template nucleic acid (e.g., template RNA) may match the target DNA upstream and downstream of the desired deletion, wherein the reverse transcription will result in the copying of the upstream and downstream sequences from the template nucleic acid (e.g., template RNA) without the intervening sequence, e.g., causing deletion of the intervening sequence. In other embodiments, the template nucleic acid (e.g., template RNA) may be designed to introduce an edit into the target DNA. For example, the template RNA may match the target DNA sequence with the exception of one or more nucleotides, wherein the reverse transcription will result in the copying of these edits into the target DNA, e.g., resulting in mutations, e.g., transition or transversion mutations.

In some embodiments, writing of an object sequence into a target site results in the substitution of nucleotides, e.g., where the full length of the object sequence corresponds to a matching length of the target site with one or more mismatched bases. In some embodiments, a heterologous object sequence may be designed such that a combination of sequence alterations may occur, e.g., a simultaneous addition and deletion, addition and substitution, or deletion and substitution.

In some embodiments, the heterologous object sequence may contain an open reading frame or a fragment of an open reading frame. In some embodiments the heterologous object sequence has a Kozak sequence. In some embodiments the heterologous object sequence has an internal ribosome entry site. In some embodiments the heterologous object sequence has a self-cleaving peptide such as a T2A or P2A site. In some embodiments the heterologous object sequence has a start codon. In some embodiments the template RNA has a splice acceptor site. In some embodiments the template RNA has a splice donor site. Exemplary splice acceptor and splice donor sites are described in WO2016044416, incorporated herein by reference in its entirety. Exemplary splice acceptor site sequences are known to those of skill in the art. In some embodiments the template RNA has a microRNA binding site downstream of the stop codon. In some embodiments the template RNA has a polyA tail downstream of the stop codon of an open reading frame. In some embodiments the template RNA comprises one or more exons. In some embodiments the template RNA comprises one or more introns. In some embodiments the template RNA comprises a eukaryotic transcriptional terminator. In some embodiments the template RNA comprises an enhanced translation element or a translation enhancing element. In some embodiments the RNA comprises the human T-cell leukemia virus (HTLV-1) R region. In some embodiments the RNA comprises a posttranscriptional regulatory element that enhances nuclear export, such as that of Hepatitis B Virus (HPRE) or Woodchuck Hepatitis Virus (WPRE).

In some embodiments, the heterologous object sequence may contain a non-coding sequence. For example, the template nucleic acid (e.g., template RNA) may comprise a regulatory element, e.g., a promoter or enhancer sequence or miRNA binding site. In some embodiments, integration of the object sequence at a target site will result in upregulation of an endogenous gene. In some embodiments, integration of the object sequence at a target site will result in downregulation of an endogenous gene. In some embodiments the template nucleic acid (e.g., template RNA) comprises a tissue specific promoter or enhancer, each of which may be unidirectional or bidirectional. In some embodiments the promoter is an RNA polymerase I promoter, RNA polymerase II promoter, or RNA polymerase III promoter. In some embodiments the promoter comprises a TATA element. In some embodiments the promoter comprises a B recognition element. In some embodiments the promoter has one or more binding sites for transcription factors.

In some embodiments, the template nucleic acid (e.g., template RNA) comprises a site that coordinates epigenetic modification. In some embodiments, the template nucleic acid (e.g., template RNA) comprises a chromatin insulator. For example, the template nucleic acid (e.g., template RNA) comprises a CTCF site or a site targeted for DNA methylation.

In some embodiments, the template nucleic acid (e.g., template RNA) comprises a gene expression unit composed of at least one regulatory region operably linked to an effector sequence. The effector sequence may be a sequence that is transcribed into RNA (e.g., a coding sequence or a non-coding sequence such as a sequence encoding a micro RNA).

In some embodiments, the heterologous object sequence of the template nucleic acid (e.g., template RNA) is inserted into a target genome in an endogenous intron. In some embodiments, the heterologous object sequence of the template nucleic acid (e.g., template RNA) is inserted into a target genome and thereby acts as a new exon. In some embodiments, the insertion of the heterologous object sequence into the target genome results in replacement of a natural exon or the skipping of a natural exon.

The template nucleic acid (e.g., template RNA) can be designed to result in insertions, mutations, or deletions at the target DNA locus. In some embodiments, the template nucleic acid (e.g., template RNA) may be designed to cause an insertion in the target DNA. For example, the template nucleic acid (e.g., template RNA) may contain a heterologous object sequence, wherein the reverse transcription will result in insertion of the heterologous object sequence into the target DNA. In other embodiments, the RNA template may be designed to write a deletion into the target DNA. For example, the template nucleic acid (e.g., template RNA) may match the target DNA upstream and downstream of the desired deletion, wherein the reverse transcription will result in the copying of the upstream and downstream sequences from the template nucleic acid (e.g., template RNA) without the intervening sequence, e.g., causing deletion of the intervening sequence. In other embodiments, the template nucleic acid (e.g., template RNA) may be designed to write an edit into the target DNA. For example, the template RNA may match the target DNA sequence with the exception of one or more nucleotides, wherein the reverse transcription will result in the copying of these edits into the target DNA, e.g., resulting in mutations, e.g., transition or transversion mutations.

In some embodiments, the pre-edit homology domain comprises a nucleic acid sequence having 100% sequence identity with a nucleic acid sequence comprised in a target nucleic acid molecule.

In some embodiments, the post-edit homology domain comprises a nucleic acid sequence having 100% sequence identity with a nucleic acid sequence comprised in a target nucleic acid molecule.

### PBS Sequence

In some embodiments, a template nucleic acid (e.g., template RNA) comprises a primer binding site (PBS) sequence. In some embodiments, a PBS sequence is disposed 3′ of the heterologous object sequence and is complementary to a sequence adjacent to a site to be modified by a system described herein, or comprises no more than 1, 2, 3, 4, or 5 mismatches to a sequence complementary to the sequence adjacent to a site to be modified by the system/heterologous gene modifying polypeptide. In some embodiments, the PBS sequence binds within 1, 2, 3, 4, 5, 6, 7, 8, 9, or 10 nucleotides of a nick site in the target nucleic acid molecule. In some embodiments, binding of the PBS sequence to the target nucleic acid molecule permits initiation of target-primed reverse transcription (TPRT), e.g., with the 3′ homology domain acting as a primer for TPRT. In some embodiments, the PBS sequence is 3-5, 5-10, 10-30, 10-25, 10-20, 10-19, 10-18, 10-17, 10-16, 10-15, 10-14, 10-13, 10-12, 10-11, 11-30, 11-25, 11-20, 11-19, 11-18, 11-17, 11-16, 11-15, 11-14, 11-13, 11-12, 12-30, 12-25, 12-20, 12-19, 12-18, 12-17, 12-16, 12-15, 12-14, 12-13, 13-30, 13-25, 13-20, 13-19, 13-18, 13-17, 13-16, 13-15, 13-14, 14-30, 14-25, 14-20, 14-19, 14-18, 14-17, 14-16, 14-15, 15-30, 15-25, 15-20, 15-19, 15-18, 15-17, 15-16, 16-30, 16-25, 16-20, 16-19, 16-18, 16-17, 17-30, 17-25, 17-20, 17-19, 17-18, 18-30, 18-25, 18-20, 18-19, 19-30, 19-25, 19-20, 20-30, 20-25, or 25-30 nucleotides in length, e.g., 10-17, 12-16, or 12-14 nucleotides in length. In some embodiments, the PBS sequence is 5-20, 8-16, 8-14, 8-13, 9-13, 9-12, or 10-12 nucleotides in length, e.g., 9-12 nucleotides in length.

The template nucleic acid (e.g., template RNA) may have some homology to the target DNA. In some embodiments, the template nucleic acid (e.g., template RNA) PBS sequence domain may serve as an annealing region to the target DNA, such that the target DNA is positioned to prime the reverse transcription of the template nucleic acid (e.g., template RNA). In some embodiments the template nucleic acid (e.g., template RNA) has at least 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 175, 200 or more bases of exact homology to the target DNA at the 3′ end of the RNA. In some embodiments the template nucleic acid (e.g., template RNA) has at least 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 175, 200 or more bases of at least 50%, 60%, 70%, 80%, 85%, 90%, 95%, 97%, 98%, 99% or 100% homology to the target DNA, e.g., at the 5′ end of the template nucleic acid (e.g., template RNA).

The template RNA sequences may be customized depending on the cell being targeted. For example, in some embodiments it is desired to inactivate a PAM sequence upon editing (e.g., using a “PAM-kill” modification) to decrease the potential for further gene editing (e.g., by Cas retargeting) following the initial edit. Consequently, certain template RNAs described herein are designed to write a mutation (e.g., a substitution) into the PAM of the target site, such that upon editing, the PAM site will be mutated to a sequence no longer recognized by the heterologous gene modifying polypeptide. Thus, a mutation region within the heterologous object sequence of the template RNA may comprise a PAM-kill sequence. Without wishing to be bound by theory, in some embodiments, a PAM-kill sequence prevents re-engagement of the heterologous gene modifying polypeptide upon completion of a genetic modification, or decreases re-engagement relative to a template RNA lacking a PAM-kill sequence. In some embodiments, a PAM-kill sequence does not alter the amino acid sequence encoded by a gene, e.g., the PAM-kill sequence results in a silent mutation. In other embodiments, it is desired to leave the PAM sequence intact (no PAM-kill).

Similarly, in some embodiments, to decrease the potential for further gene editing (e.g., by Cas retargeting) following the initial edit, it may be desirable to alter the first three nucleotides of the RT template sequence via a “seed-kill” motif. Consequently, certain template RNAs described herein are designed to write a mutation (e.g., a substitution) into the portion of the target site corresponding to the first three nucleotides of the RT template sequence, such that upon editing, the target site will be mutated to a sequence with lower homology to the RT template sequence. Thus, a mutation region within the heterologous object sequence of the template RNA may comprise a seed-kill sequence. Without wishing to be bound by theory, in some embodiments, a seed-kill sequence prevents re-engagement of the heterologous gene modifying polypeptide upon completion of genetic modification, or decreases re-engagement relative to an otherwise similar template RNA lacking a seed-kill sequence. In some embodiments, a seed-kill sequence does not alter the amino acid sequence encoded by a gene, e.g., the seed-kill sequence results in a silent mutation. In other embodiments, it is desired to leave the seed region intact, and a seed-kill sequence is not used.

In further embodiments, to optimize or improve gene editing efficiency, it may be desirable to evade the target cell's mismatch repair or nucleotide repair pathways or to bias the target cell's repair pathways toward preservation of the edited strand. In some embodiments, multiple silent mutations (for example, silent substitutions) may be introduced within the RT template sequence to evade the target cell's mismatch repair or nucleotide repair pathways or to bias the target cell's repair pathways toward preservation of the edited strand.

### Target Nucleic Acid Site

In some embodiments, after gene modification, the target site surrounding the edited sequence contains a limited number of insertions or deletions, for example, in less than about 50% or 10% of editing events, e.g., as determined by long-read amplicon sequencing of the target site, e.g., as described in Karst et al. (2020) bioRxiv doi.org/10.1101/645903 (incorporated by reference herein in its entirety). In some embodiments, the target site does not show multiple consecutive editing events, e.g., head-to-tail or head-to-head duplications, e.g., as determined by long-read amplicon sequencing of the target site, e.g., as described in Karst et al. bioRxiv doi.org/10.1101/645903 (2020) (incorporated herein by reference in its entirety). In some embodiments, the target site contains an integrated sequence corresponding to the template RNA. In some embodiments, the target site does not contain insertions resulting from endogenous RNA in more than about 1% or 10% of events, e.g., as determined by long-read amplicon sequencing of the target site, e.g., as described in Karst et al. bioRxiv doi.org/10.1101/645903 (2020) (incorporated herein by reference in its entirety). In some embodiments, the target site contains the integrated sequence corresponding to the template RNA.

In certain aspects of the present invention, the host DNA-binding site integrated into by the system can be in a gene, in an intron, in an exon, an ORF, outside of a coding region of any gene, in a regulatory region of a gene, or outside of a regulatory region of a gene. In other aspects, the polypeptide may bind to one or more than one host DNA sequence.

In some embodiments, a system is used to edit a target locus in multiple alleles. In some embodiments, a system is designed to edit a specific allele. For example, a heterologous gene modifying polypeptide may be directed to a specific sequence that is only present on one allele, e.g., comprises a template RNA with homology to a target allele, e.g., a gRNA or annealing domain, but not to a second cognate allele. In some embodiments, a heterologous gene modifying system can alter a haplotype-specific allele. In some embodiments, a heterologous gene modifying system that targets a specific allele preferentially targets that allele, e.g., has at least a 2, 4, 6, 8, or 10-fold preference for a target allele.

### Second Strand Nicking

In some embodiments, a heterologous gene modifying system described herein comprises a nickase activity (e.g., in the heterologous gene modifying polypeptide) that nicks the first strand, and a nickase activity (e.g., in a polypeptide separate from the heterologous gene modifying polypeptide) that nicks the second strand of target DNA. As discussed herein, without wishing to be bound by theory, nicking of the first strand of the target site DNA is thought to provide a 3′ OH that can be used by an RT domain to reverse transcribe a sequence of a template RNA, e.g., a heterologous object sequence. Without wishing to be bound by theory, it is thought that introducing an additional nick to the second strand may bias the cellular DNA repair machinery to adopt the heterologous object sequence-based sequence more frequently than the original genomic sequence. In some embodiments, the additional nick to the second strand is made by the same endonuclease domain (e.g., nickase domain) as the nick to the first strand. In some embodiments, the same heterologous gene modifying polypeptide performs both the nick to the first strand and the nick to the second strand. In some embodiments, the heterologous gene modifying polypeptide comprises a CRISPR/Cas domain and the additional nick to the second strand is directed by an additional nucleic acid, e.g., comprising a second gRNA directing the CRISPR/Cas domain to nick the second strand. In other embodiments, the additional second strand nick is made by a different endonuclease domain (e.g., nickase domain) than the nick to the first strand. In some embodiments, that different endonuclease domain is situated in an additional polypeptide (e.g., a system of the invention further comprises the additional polypeptide), separate from the heterologous gene modifying polypeptide. In some embodiments, the additional polypeptide comprises an endonuclease domain (e.g., nickase domain) described herein. In some embodiments, the additional polypeptide comprises a DNA binding domain, e.g., described herein.

It is contemplated herein that the position at which the second strand nick occurs relative to the first strand nick may influence the extent to which one or more of: desired gene modifying DNA modifications are obtained, undesired double-strand breaks (DSBs) occur, undesired insertions occur, or undesired deletions occur. Without wishing to be bound by theory, second strand nicking may occur in two general orientations: inward nicks and outward nicks.

In some embodiments, in the inward nick orientation, the RT domain polymerizes (e.g., using the template RNA (e.g., the heterologous object sequence)) away from the second strand nick. In some embodiments, in the inward nick orientation, the location of the nick to the first strand and the location of the nick to the second strand are positioned between the first PAM site and second PAM site (e.g., in a scenario wherein both nicks are made by a polypeptide (e.g., a heterologous gene modifying polypeptide) comprising a CRISPR/Cas domain). When there are two PAMs on the outside and two nicks on the inside, this inward nick orientation can also be referred to as “PAM-out”. In some embodiments, in the inward nick orientation, the location of the nick to the first strand and the location of the nick to the second strand are between the sites where the polypeptide and the additional polypeptide bind to the target DNA. In some embodiments, in the inward nick orientation, the location of the nick to the second strand is positioned between the binding sites of the polypeptide and additional polypeptide, and the nick to the first strand is also located between the binding sites of the polypeptide and additional polypeptide. In some embodiments, in the inward nick orientation, the location of the nick to the first strand and the location of the nick to the second strand are positioned between the PAM site and the binding site of the second polypeptide which is at a distance from the target site.

An example of a heterologous gene modifying system that provides an inward nick orientation comprises a heterologous gene modifying polypeptide comprising a CRISPR/Cas domain, a template RNA comprising a gRNA that directs nicking of the target site DNA on the first strand, and an additional nucleic acid comprising an additional gRNA that directs nicking at a site a distance from the location of the first nick, wherein the location of the first nick and the location of the second nick are between the PAM sites of the sites to which the two gRNAs direct the heterologous gene modifying polypeptide. As a further example, another heterologous gene modifying system that provides an inward nick orientation comprises a heterologous gene modifying polypeptide comprising a zinc finger molecule and a first nickase domain wherein the zinc finger molecule binds to the target DNA in a manner that directs the first nickase domain to nick the first strand of the target site; an additional polypeptide comprising a CRISPR/Cas domain, and an additional nucleic acid comprising a gRNA that directs the additional polypeptide to nick a site a distance from the target site DNA on the second strand, wherein the location of the first nick and the location of the second nick are between the PAM site and the site to which the zinc finger molecule binds. As a further example, another heterologous gene modifying system that provides an inward nick orientation comprises a heterologous gene modifying polypeptide comprising a zinc finger molecule and a first nickase domain wherein the zinc finger molecule binds to the target DNA in a manner that directs the first nickase domain to nick the first strand of the target site; an additional polypeptide comprising a TAL effector molecule and a second nickase domain wherein the TAL effector molecule binds to a site a distance from the target site in a manner that directs the additional polypeptide to nick the second strand, wherein the location of the first nick and the location of the second nick are between the site to which the TAL effector molecule binds and the site to which the zinc finger molecule binds.

In some embodiments, in the outward nick orientation, the RT domain polymerizes (e.g., using the template RNA (e.g., the heterologous object sequence)) toward the second strand nick. In some embodiments, in the outward nick orientation when both the first and second nicks are made by a polypeptide comprising a CRISPR/Cas domain (e.g., a heterologous gene modifying polypeptide), the first PAM site and second PAM site are positioned between the location of the nick to the first strand and the location of the nick to the second strand. When there are two PAMs on the inside and two nicks on the outside, this outward nick orientation also can be referred to as “PAM-in”. In some embodiments, in the outward nick orientation, the polypeptide (e.g., the heterologous gene modifying polypeptide) and the additional polypeptide bind to sites on the target DNA between the location of the nick to the first strand and the location of the nick to the second. In some embodiments, in the outward nick orientation, the location of the nick to the second strand is positioned on the opposite side of the binding sites of the polypeptide and additional polypeptide relative to the location of the nick to the first strand. In some embodiments, in the outward orientation, the PAM site and the binding site of the second polypeptide which is at a distance from the target site are positioned between the location of the nick to the first strand and the location of the nick to the second strand.

An example of a heterologous gene modifying system that provides an outward nick orientation comprises a heterologous gene modifying polypeptide comprising a CRISPR/Cas domain, a template RNA comprising a gRNA that directs nicking of the target site DNA on the first strand, and an additional nucleic acid comprising an additional gRNA that directs nicking at a site a distance from the location of the first nick, wherein the location of the first nick and the location of the second nick are outside of the PAM sites of the sites to which the two gRNAs direct the heterologous gene modifying polypeptide (i.e., the PAM sites are between the location of the first nick and the location of the second nick). As a further example, another heterologous gene modifying system that provides an outward nick orientation comprises a heterologous gene modifying polypeptide comprising a zinc finger molecule and a first nickase domain wherein the zinc finger molecule binds to the target DNA in a manner that directs the first nickase domain to nick the first strand of the target site; an additional polypeptide comprising a CRISPR/Cas domain, and an additional nucleic acid comprising a gRNA that directs the additional polypeptide to nick a site a distance from the target site DNA on the second strand, wherein the location of the first nick and the location of the second nick are outside the PAM site and the site to which the zinc finger molecule binds (i.e., the PAM site and the site to which the zinc finger molecule binds are between the location of the first nick and the location of the second nick). As a further example, another heterologous gene modifying system that provides an outward nick orientation comprises a heterologous gene modifying polypeptide comprising a zinc finger molecule and a first nickase domain wherein the zinc finger molecule binds to the target DNA in a manner that directs the first nickase domain to nick the first strand of the target site; an additional polypeptide comprising a TAL effector molecule and a second nickase domain wherein the TAL effector molecule binds to a site a distance from the target site in a manner that directs the additional polypeptide to nick the second strand, wherein the location of the first nick and the location of the second nick are outside the site to which the TAL effector molecule binds and the site to which the zinc finger molecule binds (i.e., the site to which the TAL effector molecule binds and the site to which the zinc finger molecule binds are between the location of the first nick and the location of the second nick).

Without wishing to be bound by theory, it is thought that, for heterologous gene modifying systems where a second strand nick is provided, an outward nick orientation is preferred in some embodiments. As is described herein, an inward nick may produce a higher number of double-strand breaks (DSBs) than an outward nick orientation. DSBs may be recognized by the DSB repair pathways in the nucleus of a cell, which can result in undesired insertions and deletions. An outward nick orientation may provide a decreased risk of DSB formation, and a corresponding lower amount of undesired insertions and deletions. In some embodiments, undesired insertions and deletions are insertions and deletions not encoded by the heterologous object sequence, e.g., an insertion or deletion produced by the double-strand break repair pathway unrelated to the modification encoded by the heterologous object sequence. In some embodiments, a desired gene modification comprises a change to the target DNA (e.g., a substitution, insertion, or deletion) encoded by the heterologous object sequence (e.g., and achieved by the heterologous gene modifying writing the heterologous object sequence into the target site). In some embodiments, the first strand nick and the second strand nick are in an outward orientation.

In addition, the distance between the first strand nick and second strand nick may influence the extent to which one or more of: desired heterologous gene modifying system DNA modifications are obtained, undesired double-strand breaks (DSBs) occur, undesired insertions occur, or undesired deletions occur. Without wishing to be bound by theory, it is thought the second strand nick benefit, the biasing of DNA repair toward incorporation of the heterologous object sequence into the target DNA, increases as the distance between the first strand nick and second strand nick decreases. However, it is thought that the risk of DSB formation also increases as the distance between the first strand nick and second strand nick decreases. Correspondingly, it is thought that the number of undesired insertions and/or deletions may increase as the distance between the first strand nick and second strand nick decreases. In some embodiments, the distance between the first strand nick and second strand nick is chosen to balance the benefit of biasing DNA repair toward incorporation of the heterologous object sequence into the target DNA and the risk of DSB formation and of undesired deletions and/or insertions. In some embodiments, a system where the first strand nick and the second strand nick are at least a threshold distance apart has an increased level of desired heterologous gene modifying system modification outcomes, a decreased level of undesired deletions, and/or a decreased level of undesired insertions relative to an otherwise similar inward nick orientation system where the first nick and the second nick are less than the a threshold distance apart. In some embodiments the threshold distance(s) is given below.

In some embodiments, the first nick and the second nick are at least 20, 25, 30, 35, 40, 45, 50, 55, 60, 65, 70, 75, 80, 85, 90, 95, 100, 110, 120, 130, 140, 150, 160, 170, 180, 190, or 200 nucleotides apart. In some embodiments, the first nick and the second nick are no more than 25, 30, 35, 40, 45, 50, 55, 60, 65, 70, 75, 80, 85, 90, 95, 100, 110, 120, 130, 140, 150, 160, 170, 180, 190, 200, or 250 nucleotides apart. In some embodiments, the first nick and the second nick are 20-200, 30-200, 40-200, 50-200, 60-200, 70-200, 80-200, 90-200, 100-200, 110-200, 120-200, 130-200, 140-200, 150-200, 160-200, 170-200, 180-200, 190-200, 20-190, 30-190, 40-190, 50-190, 60-190, 70-190, 80-190, 90-190, 100-190, 110-190, 120-190, 130-190, 140-190, 150-190, 160-190, 170-190, 180-190, 20-180, 30-180, 40-180, 50-180, 60-180, 70-180, 80-180, 90-180, 100-180, 110-180, 120-180, 130-180, 140-180, 150-180, 160-180, 170-180, 20-170, 30-170, 40-170, 50-170, 60-170, 70-170, 80-170, 90-170, 100-170, 110-170, 120-170, 130-170, 140-170, 150-170, 160-170, 20-160, 30-160, 40-160, 50-160, 60-160, 70-160, 80-160, 90-160, 100-160, 110-160, 120-160, 130-160, 140-160, 150-160, 20-150, 30-150, 40-150, 50-150, 60-150, 70-150, 80-150, 90-150, 100-150, 110-150, 120-150, 130-150, 140-150, 20-140, 30-140, 40-140, 50-140, 60-140, 70-140, 80-140, 90-140, 100-140, 110-140, 120-140, 130-140, 20-130, 30-130, 40-130, 50-130, 60-130, 70-130, 80-130, 90-130, 100-130, 110-130, 120-130, 20-120, 30-120, 40-120, 50-120, 60-120, 70-120, 80-120, 90-120, 100-120, 110-120, 20-110, 30-110, 40-110, 50-110, 60-110, 70-110, 80-110, 90-110, 100-110, 20-100, 30-100, 40-100, 50-100, 60-100, 70-100, 80-100, 90-100, 20-90, 30-90, 40-90, 50-90, 60-90, 70-90, 80-90, 20-80, 30-80, 40-80, 50-80, 60-80, 70-80, 20-70, 30-70, 40-70, 50-70, 60-70, 20-60, 30-60, 40-60, 50-60, 20-50, 30-50, 40-50, 20-40, 30-40, or 20-30 nucleotides apart. In some embodiments, the first nick and the second nick are 40-100 nucleotides apart.

Without wishing to be bound by theory, it is thought that, for heterologous gene modifying systems where a second strand nick is provided and an inward nick orientation is selected, increasing the distance between the first strand nick and second strand nick may be preferred. As is described herein, an inward nick orientation may produce a higher number of DSBs than an outward nick orientation, and may result in a higher amount of undesired insertions and deletions than an outward nick orientation, but increasing the distance between the nicks may mitigate that increase in DSBs, undesired deletions, and/or undesired insertions. In some embodiments, an inward nick orientation wherein the first nick and the second nick are at least a threshold distance apart has an increased level of desired heterologous gene modifying system modification outcomes, a decreased level of undesired deletions, and/or a decreased level of undesired insertions relative to an otherwise similar inward nick orientation system where the first nick and the second nick are less than the a threshold distance apart. In some embodiments the threshold distance is given below.

In some embodiments, the first strand nick and the second strand nick are in an inward orientation. In some embodiments, the first strand nick and the second strand nick are in an inward orientation and the first strand nick and second strand nick are at least 100, 110, 120, 130, 140, 150, 160, 170, 180, 190, 200, 220, 240, 260, 280, 300, 350, 400, 450, or 500 nucleotides apart, e.g., at least 100 nucleotides apart, (and optionally no more than 500, 400, 300, 200, 190, 180, 170, 160, 150, 140, 130, or 120 nucleotides apart). In some embodiments, the first strand nick and the second strand nick are in an inward orientation and the first strand nick and second strand nick are 100-200, 110-200, 120-200, 130-200, 140-200, 150-200, 160-200, 170-200, 180-200, 190-200, 100-190, 110-190, 120-190, 130-190, 140-190, 150-190, 160-190, 170-190, 180-190, 100-180, 110-180, 120-180, 130-180, 140-180, 150-180, 160-180, 170-180, 100-170, 110-170, 120-170, 130-170, 140-170, 150-170, 160-170, 100-160, 110-160, 120-160, 130-160, 140-160, 150-160, 100-150, 110-150, 120-150, 130-150, 140-150, 100-140, 110-140, 120-140, 130-140, 100-130, 110-130, 120-130, 100-120, 110-120, or 100-110 nucleotides apart.

### Production of Compositions and Systems

Methods of designing and constructing nucleic acid constructs and proteins or polypeptides (such as the systems, constructs, and polypeptides described herein) are known. Generally, recombinant methods may be used. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013). Methods of designing, preparing, evaluating, purifying and manipulating nucleic acid compositions are described in Green and Sambrook (Eds.), Molecular Cloning: A Laboratory Manual (Fourth Edition), Cold Spring Harbor Laboratory Press (2012).

The disclosure provides, in part, a nucleic acid, e.g., vector, encoding a gene modifying polypeptide described herein, a template nucleic acid described herein, or both. In some embodiments, a vector comprises a selective marker, e.g., an antibiotic resistance marker. In some embodiments, the antibiotic resistance marker is a kanamycin resistance marker. In some embodiments, the antibiotic resistance marker does not confer resistance to beta-lactam antibiotics. In some embodiments, the vector does not comprise an ampicillin resistance marker. In some embodiments, the vector comprises a kanamycin resistance marker and does not comprise an ampicillin resistance marker. In some embodiments, a vector encoding a gene modifying polypeptide is integrated into a target cell genome (e.g., upon administration to a target cell, tissue, organ, or subject). In some embodiments, a vector encoding a gene modifying polypeptide is not integrated into a target cell genome (e.g., upon administration to a target cell, tissue, organ, or subject). In some embodiments, a vector encoding a template nucleic acid (e.g., template RNA) is not integrated into a target cell genome (e.g., upon administration to a target cell, tissue, organ, or subject). In some embodiments, if a vector is integrated into a target site in a target cell genome, the selective marker is not integrated into the genome. In some embodiments, if a vector is integrated into a target site in a target cell genome, genes or sequences involved in vector maintenance (e.g., plasmid maintenance genes) are not integrated into the genome. In some embodiments, if a vector is integrated into a target site in a target cell genome, transfer regulating sequences (e.g., inverted terminal repeats, e.g., from an AAV) are not integrated into the genome. In some embodiments, administration of a vector (e.g., encoding a gene modifying polypeptide described herein, a template nucleic acid described herein, or both) to a target cell, tissue, organ, or subject results in integration of a portion of the vector into one or more target sites in the genome(s) of said target cell, tissue, organ, or subject. In some embodiments, less than 99, 95, 90, 80, 70, 60, 50, 40, 30, 20, 10, 5, 4, 3, 2, or 1% of target sites (e.g., no target sites) comprising integrated material comprise a selective marker (e.g., an antibiotic resistance gene), a transfer regulating sequence (e.g., an inverted terminal repeat, e.g., from an AAV), or both from the vector.

Exemplary methods for producing a therapeutic pharmaceutical protein or polypeptide described herein involve expression in mammalian cells, although recombinant proteins can also be produced using insect cells, yeast, bacteria, or other cells under control of appropriate promoters. Mammalian expression vectors may comprise non-transcribed elements such as an origin of replication, a suitable promoter, and other 5′ or 3′ flanking non-transcribed sequences, and 5′ or 3′ non-translated sequences such as necessary ribosome binding sites, a polyadenylation site, splice donor and acceptor sites, and termination sequences. DNA sequences derived from the SV40 viral genome, for example, SV40 origin, early promoter, splice, and polyadenylation sites may be used to provide other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press (2012).

Various mammalian cell culture systems can be employed to express and manufacture recombinant protein. Examples of mammalian expression systems include CHO, COS, HEK293, HeLA, and BHK cell lines. Processes of host cell culture for production of protein therapeutics are described in Zhou and Kantardjieff (Eds.), Mammalian Cell Cultures for Biologics Manufacturing (Advances in Biochemical Engineering Biotechnology), Springer (2014). Compositions described herein may include a vector, such as a viral vector, e.g., a lentiviral vector, encoding a recombinant protein. In some embodiments, a vector, e.g., a viral vector, may comprise a nucleic acid encoding a recombinant protein.

Purification of protein therapeutics is described in Franks, Protein Biotechnology. Isolation, Characterization, and Stabilization, Humana Press (2013); and in Cutler, Protein Purification Protocols (Methods in Molecular Biology), Humana Press (2010).

In some embodiments, quality standards include, but are not limited to:
(i) the length of mRNA encoding the gene modifying polypeptide, e.g., whether the mRNA has a length that is above a reference length or within a reference length range, e.g., whether at least 80, 85, 90, 95, 96, 97, 98, or 99% of the mRNA present is greater than 3000, 4000, or 5000 nucleotides long;
(ii) the presence, absence, and/or length of a polyA tail on the mRNA, e.g., whether at least 80, 85, 90, 95, 96, 97, 98, or 99% of the mRNA present contains a polyA tail (e.g., a polyA tail that is at least 5, 10, 20, 30, 50, 70, 100 nucleotides in length);
(iii) the presence, absence, and/or type of a 5′ cap on the mRNA, e.g., whether at least 80, 85, 90, 95, 96, 97, 98, or 99% of the mRNA present contains a 5′ cap, e.g., whether that cap is a 7-methylguanosine cap, e.g., a O-Me-m7G cap;
(iv) the presence, absence, and/or type of one or more modified nucleotides (e.g., selected from pseudouridine, dihydrouridine, inosine, 7-methylguanosine, 1-N-methylpseudouridine (1-Me-Ψ), 5-methoxyuridine (5-MO-U), 5-methylcytidine (5mC), or a locked nucleotide) in the mRNA, e.g., whether at least 80, 85, 90, 95, 96, 97, 98, or 99% of the mRNA present contains one or more modified nucleotides;
(v) the stability of the mRNA (e.g., over time and/or under a pre-selected condition), e.g., whether at least 80, 85, 90, 95, 96, 97, 98, or 99% of the mRNA remains intact (e.g., greater than 100, 125, 150, 175, or 200 nucleotides long) after a stability test; or
(vi) the potency of the mRNA in a system for modifying DNA, e.g., whether at least 1% of target sites are modified after a system comprising the mRNA is assayed for potency.

### Kits, Articles of Manufacture, and Pharmaceutical Compositions

In an aspect the disclosure provides a kit comprising a gene modifying polypeptide or a gene modifying system, e.g., as described herein. In some embodiments, the kit comprises a gene modifying polypeptide (or a nucleic acid encoding the polypeptide) and a template RNA (or DNA encoding the template RNA). In some embodiments, the kit further comprises a reagent for introducing the system into a cell, e.g., transfection reagent, LNP, and the like. In some embodiments, the kit is suitable for any of the methods described herein. In some embodiments, the kit comprises one or more elements, compositions (e.g., pharmaceutical compositions), gene modifying polypeptides, and/or gene modifying systems, or a functional fragment or component thereof, e.g., disposed in an article of manufacture. In some embodiments, the kit comprises instructions for use thereof.

In an aspect, the disclosure provides an article of manufacture, e.g., in which a kit as described herein, or a component thereof, is disposed.

In an aspect, the disclosure provides a pharmaceutical composition comprising a gene modifying polypeptide or a gene modifying system, e.g., as described herein. In some embodiments, the pharmaceutical composition further comprises a pharmaceutically acceptable carrier or excipient. In some embodiments, the pharmaceutical composition comprises a template RNA and/or an RNA encoding the polypeptide. In embodiments, the pharmaceutical composition has one or more (e.g., 1, 2, 3, or 4) of the following characteristics:
(a) less than 1% (e.g., less than 0.5%, 0.4%, 0.3%, 0.2%, or 0.1%) DNA template relative to the template RNA and/or the RNA encoding the polypeptide, e.g., on a molar basis;
(b) less than 1% (e.g., less than 0.5%, 0.4%, 0.3%, 0.2%, or 0.1%) uncapped RNA relative to the template RNA and/or the RNA encoding the polypeptide, e.g., on a molar basis;
(c) less than 1% (e.g., less than 0.5%, 0.4%, 0.3%, 0.2%, or 0.1%) partial length RNAs relative to the template RNA and/or the RNA encoding the polypeptide, e.g., on a molar basis;
(d) substantially lacks unreacted cap dinucleotides.

### Chemistry, Manufacturing, and Controls (CMC)

Purification of protein therapeutics is described, for example, in Franks, Protein Biotechnology: Isolation, Characterization, and Stabilization, Humana Press (2013); and in Cutler, Protein Purification Protocols (Methods in Molecular Biology), Humana Press (2010).

In some embodiments, a gene modifying system, polypeptide, and/or template nucleic acid (e.g., template RNA) conforms to certain quality standards. In some embodiments, a gene modifying system, polypeptide, and/or template nucleic acid (e.g., template RNA) produced by a method described herein conforms to certain quality standards. Accordingly, the disclosure is directed, in some aspects, to methods of manufacturing a gene modifying system, polypeptide, and/or template nucleic acid (e.g., template RNA) that conforms to certain quality standards, e.g., in which said quality standards are assayed. The disclosure is also directed, in some aspects, to methods of assaying said quality standards in a gene modifying system, polypeptide, and/or template nucleic acid (e.g., template RNA). In some embodiments, quality standards include, but are not limited to, one or more (e.g., 1, 2, 3, 4, 5, 6, 7, 8, 9, 10, 11, or 12) of the following:
(i) the length of the template RNA, e.g., whether the template RNA has a length that is above a reference length or within a reference length range, e.g., whether at least 80, 85, 90, 95, 96, 97, 98, or 99% of the template RNA present is greater than 100, 125, 150, 175, or 200 nucleotides long;
(ii) the presence, absence, and/or length of a polyA tail on the template RNA, e.g., whether at least 80, 85, 90, 95, 96, 97, 98, or 99% of the template RNA present contains a polyA tail (e.g., a polyA tail that is at least 5, 10, 20, 30, 50, 70, 100 nucleotides in length (SEQ ID NO: 15550));
(iii) the presence, absence, and/or type of a 5′ cap on the template RNA, e.g., whether at least 80, 85, 90, 95, 96, 97, 98, or 99% of the template RNA present contains a 5′ cap, e.g., whether that cap is a 7-methylguanosine cap, e.g., a O-Me-m7G cap;
(iv) the presence, absence, and/or type of one or more modified nucleotides (e.g., selected from pseudouridine, dihydrouridine, inosine, 7-methylguanosine, 1-N-methylpseudouridine (1-Me-Ψ), 5-methoxyuridine (5-MO-U), 5-methylcytidine (5mC), or a locked nucleotide) in the template RNA, e.g., whether at least 80, 85, 90, 95, 96, 97, 98, or 99% of the template RNA present contains one or more modified nucleotides;
(v) the stability of the template RNA (e.g., over time and/or under a pre-selected condition), e.g., whether at least 80, 85, 90, 95, 96, 97, 98, or 99% of the template RNA remains intact (e.g., greater than 100, 125, 150, 175, or 200 nucleotides long) after a stability test;
(vi) the potency of the template RNA in a system for modifying DNA, e.g., whether at least 1% of target sites are modified after a system comprising the template RNA is assayed for potency;
(vii) the length of the polypeptide, first polypeptide, or second polypeptide, e.g., whether the polypeptide, first polypeptide, or second polypeptide has a length that is above a reference length or within a reference length range, e.g., whether at least 80, 85, 90, 95, 96, 97, 98, or 99% of the polypeptide, first polypeptide, or second polypeptide present is greater than 600, 650, 700, 750, 800, 850, 900, 950, 1000, 1050, 1100, 1150, 1200, 1250, 1300, 1350, 1400, 1450, 1500, 1600, 1700, 1800, 1900, or 2000 amino acids long (and optionally, no larger than 2500, 2000, 1500, 1400, 1300, 1200, 1100, 1000, 900, 800, 700, or 600 amino acids long);
(viii) the presence, absence, and/or type of post-translational modification on the polypeptide, first polypeptide, or second polypeptide, e.g., whether at least 80, 85, 90, 95, 96, 97, 98, or 99% of the polypeptide, first polypeptide, or second polypeptide contains phosphorylation, methylation, acetylation, myristoylation, palmitoylation, isoprenylation, glipyatyon, or lipoylation, or any combination thereof;
(ix) the presence, absence, and/or type of one or more artificial, synthetic, or non-canonical amino acids (e.g., selected from ornithine, β-alanine, GABA, δ-Aminolevulinic acid, PABA, a D-amino acid (e.g., D-alanine or D-glutamate), aminoisobutyric acid, dehydroalanine, cystathionine, lanthionine, Djenkolic acid, Diaminopimelic acid, Homoalanine, Norvaline, Norleucine, Homonorleucine, homoserine, O-methyl-homoserine and O-ethyl-homoserine, ethionine, selenocysteine, selenohomocysteine, selenomethionine, selenoethionine, tellurocysteine, or telluromethionine) in the polypeptide, first polypeptide, or second polypeptide, e.g., whether at least 80, 85, 90, 95, 96, 97, 98, or 99% of the polypeptide, first polypeptide, or second polypeptide present contains one or more artificial, synthetic, or non-canonical amino acids;
(x) the stability of the polypeptide, first polypeptide, or second polypeptide (e.g., over time and/or under a pre-selected condition), e.g., whether at least 80, 85, 90, 95, 96, 97, 98, or 99% of the polypeptide, first polypeptide, or second polypeptide remains intact (e.g., greater than 600, 650, 700, 750, 800, 850, 900, 950, 1000, 1050, 1100, 1150, 1200, 1250, 1300, 1350, 1400, 1450, 1500, 1600, 1700, 1800, 1900, or 2000 amino acids long (and optionally, no larger than 2500, 2000, 1500, 1400, 1300, 1200, 1100, 1000, 900, 800, 700, or 600 amino acids long)) after a stability test;
(xi) the potency of the polypeptide, first polypeptide, or second polypeptide in a system for modifying DNA, e.g., whether at least 1% of target sites are modified after a system comprising the polypeptide, first polypeptide, or second polypeptide is assayed for potency; or
(xii) the presence, absence, and/or level of one or more of a pyrogen, virus, fungus, bacterial pathogen, or host cell protein, e.g., whether the system is free or substantially free of pyrogen, virus, fungus, bacterial pathogen, or host cell protein contamination.

In some embodiments, a system or pharmaceutical composition described herein is endotoxin free.

In some embodiments, the presence, absence, and/or level of one or more of a pyrogen, virus, fungus, bacterial pathogen, and/or host cell protein is determined. In embodiments, whether the system is free or substantially free of pyrogen, virus, fungus, bacterial pathogen, and/or host cell protein contamination is determined.

In some embodiments, a pharmaceutical composition or system as described herein has one or more (e.g., 1, 2, 3, or 4) of the following characteristics:
(a) less than 1% (e.g., less than 0.5%, 0.4%, 0.3%, 0.2%, or 0.1%) DNA template relative to the template RNA and/or the RNA encoding the polypeptide, e.g., on a molar basis;
(b) less than 1% (e.g., less than 0.5%, 0.4%, 0.3%, 0.2%, or 0.1%) uncapped RNA relative to the template RNA and/or the RNA encoding the polypeptide, e.g., on a molar basis;
(c) less than 1% (e.g., less than 0.5%, 0.4%, 0.3%, 0.2%, or 0.1%) partial length RNAs relative to the template RNA and/or the RNA encoding the polypeptide, e.g., on a molar basis;
(d) substantially lacks unreacted cap dinucleotides.

### Applications

By integrating coding genes into a RNA sequence template, the gene modifying system can address therapeutic needs, for example, by providing expression of a therapeutic transgene in individuals with loss-of-function mutations, by replacing gain-of-function mutations with normal transgenes, by providing regulatory sequences to eliminate gain-of-function mutation expression, and/or by controlling the expression of operably linked genes, transgenes and systems thereof. In certain embodiments, the RNA sequence template encodes a promotor region specific to the therapeutic needs of the host cell, for example a tissue specific promotor or enhancer. In still other embodiments, a promotor can be operably linked to a coding sequence.

In embodiments, the gene modifying system can provide therapeutic transgenes expressing, e.g., replacement blood factors or replacement enzymes, e.g., lysosomal enzymes. For example, the compositions, systems and methods described herein are useful to express, in a target human genome, agalsidase alpha or beta for treatment of Fabry Disease; imiglucerase, taliglucerase alfa, velaglucerase alfa, or alglucerase for Gaucher Disease; sebelipase alpha for lysosomal acid lipase deficiency (Wolman disease/CESD); laronidase, idursulfase, elosulfase alpha, or galsulfase for mucopolysaccharidoses; alglucosidase alpha for Pompe disease. For example, the compositions, systems, and methods described herein are useful to express, in a target human genome factor I, II, V, VII, X, XI, XII or XIII for blood factor deficiencies.

In some embodiments, the heterologous object sequence encodes an intracellular protein (e.g., a cytoplasmic protein, a nuclear protein, an organellar protein such as a mitochondrial protein or lysosomal protein, or a membrane protein). In some embodiments, the heterologous object sequence encodes a membrane protein, e.g., a CAR or a membrane protein other than a CAR, and/or an endogenous human membrane protein. In some embodiments, the heterologous object sequence encodes an extracellular protein. In some embodiments, the heterologous object sequence encodes an enzyme, a structural protein, a signaling protein, a regulatory protein, a transport protein, a sensory protein, a motor protein, a defense protein, or a storage protein. Other exemplary proteins that may be encoded by a heterologous object sequence include, without limitation, an immune receptor protein, e.g. a synthetic immune receptor protein such as a chimeric antigen receptor protein (CAR), a T cell receptor, a B cell receptor, or an antibody.

### Chimeric Antigen Receptors

In some embodiments, the heterologous object sequence encodes a chimeric antigen receptor (CAR) comprising an antigen binding domain. In some embodiments, the CAR is or comprises a first generation CAR comprising an antigen binding domain, a transmembrane domain, and a single intracellular signaling domain. In some embodiments, the CAR is or comprises a second generation CAR comprising an antigen binding domain, a transmembrane domain, and two intracellular signaling domains (e.g., a first intracellular signaling domain and a second intracellular signaling domain). In some embodiments, the CAR comprises a third generation CAR comprising an antigen binding domain, a transmembrane domain, and at least three intracellular signaling domains. In some embodiments, a fourth generation CAR comprising an antigen binding domain, a transmembrane domain, three or four intracellular signaling domains, and a domain which upon successful signaling of the CAR induces expression of a cytokine gene. In some embodiments, the antigen binding domain is or comprises an scFv, Fab, a diabody, a D domain binder, centyrins (e.g., antibody-like scaffolds, e.g., a CARTyrin), one or more single domain antibodies such as VHH domains (e.g., comprises two VHH binding domains). In some embodiments, the CAR antigen binding domain binds to two epitopes of the target antigen (e.g., is a biepitopic binding domain). In some embodiments, the CAR comprises two antigen binding domains, such that each antigen binding domain binds to a different target antigen on a cell, e.g., a neoplastic cell.

### Antigen Binding Domains

In some embodiments, a CAR antigen binding domain is or comprises an antibody or antigen-binding portion thereof. In some embodiments, a CAR antigen binding domain is or comprises an scFv, Fab, a diabody, a D domain binder, centyrins (e.g., antibody-like scaffolds, e.g., a CARTyrin), one or more single domain antibodies such as VHH domains (e.g., comprises two VHH binding domains). In some embodiments, the CAR antigen binding domain binds to two epitopes of the target antigen (e.g., is a biepitopic binding domain). In some embodiments, the CAR comprises two antigen binding domains, such that each antigen binding domain binds to a different target antigen on a cell, e.g., a neoplastic cell.

In some embodiments, the CAR comprises a camelid antigen-binding domain. In some embodiments, the CAR comprises a murine binding domain. In some embodiments, the CAR comprises a humanized binding domain. In some embodiments, the CAR comprises a human binding domain.

In some embodiments, an antigen binding domain binds to a cell surface antigen of a cell. In some embodiments, a cell surface antigen is characteristic of one type of cell. In some embodiments, a cell surface antigen is characteristic of more than one type of cell. In some embodiments, the antigen binding domain targets an antigen characteristic of a neoplastic cell. In some embodiments, the antigen characteristic of a neoplastic cell is selected from a receptor listed in Table C1, or an antigenic fragment or antigenic portion thereof. In some embodiments, the antigen binding domain binds one or more antigens of a blood cancer (e.g., a leukemia, a lymphoma, or a multiple myeloma). In some embodiments, the blood cancer antigen is a B cell antigen. In some embodiments, the antigen is BCMA. In some embodiments, the antigen is GPRC5D. In some embodiments, the antigen is CD20. In some embodiments, the antigen binding domain binds an antigen of a solid tumor.

TABLE C1
Exemplary Neoplastic Cell Antigens

| | | | | |
|---|---|---|---|---|
| cell surface | NAV1.6 | Carbonic | prostate | p53 mutant |
| receptor | | anhydrase 1X | specific | |
| | | | membrane | |
| | | | antigen | |
| | | | (PSMA) | |
| an ion | NAV1.7 | PSMA | ROR1 | prostein |
| channel- | | | | |
| linked | | | | |
| receptor | | | | |
| an enzyme- | NAV1.8 | Folate | FLT3 | survivin |
| linked | | binding | | |
| receptor | | protein | | |
| a G protein- | NAV1.9 | Gangliosides | FAP | telomerase |
| coupled | | | | |
| receptor | | | | |
| receptor | sphingosin-1- | CD2 | TAG72 | PCTA- |
| tyrosine | phosphate | | | 1/Galectin 8 |
| kinase | receptor | | | |
| | (S1P1R) | | | |
| tyrosine | NMDA | CD3 | CD38 | MelanA/MART1 |
| kinase | channel | | | |
| associated | | | | |
| receptor | | | | |
| receptor-like | transmembrane | GM2) | CD44v6 | Ras mutant |
| tyrosine | protein | | | |
| phosphatase | | | | |
| receptor | multispan | Lewis-γ² | CEA | hTERT |
| serine/ | transmembrane | | | |
| threonine | protein | | | |
| kinase | | | | |
| receptor | T-cell receptor | VEGF | EPCAM | sarcoma |
| guanylyl | motifs | | | translocation |
| cyclase | | | | breakpoints |
| histidine | T-cell alpha | VEGFR | B7H3 | ML-IAP |
| kinase | chains | 1/2/3 | | |
| associated | | | | |
| receptor | | | | |
| ErbB1/EGFR | T-cell β chains | αVβ3 | KIT | ERG |
| | | | | (TMPRSS2 ETS |
| | | | | fusion gene) |
| ErbB2/HER2 | T-cell γ chains | α5β1 | interleukin-11 | NA17 |
| | | | receptor a (IL- | |
| | | | 11Ra) | |
| ErbB3/HER3 | T-cell δ chains | ErbB1/EGFR | PSCA | PAX3 |
| and | CCR7 | ErbB1/HER2 | PRSS21 | Androgen |
| ErbB4/HER4) | | | | receptor |
| FGF1 | CD3 | ErB3 | VEGFR2 | Cyclin B1 |
| FGF2 | CD4 | c-MET | LewisY | MYCN |
| FGF3 | CD5 | IGF1R | CD24 | RhoC |
| FGF4 | CD7 | EphA3 | platelet- | TRP-2 |
| | | | derived | |
| | | | growth factor | |
| | | | receptor-beta | |
| | | | (PDGFR-beta) | |
| FGF5 | CD8 | TRAIL-R1 | SSEA-4 | CYPIB I |
| FGF6 | CD11b | TRAIL-R2 | CD20 | BORIS |
| FGF7 | CD11c | RANKL | MUC1 | SART3 |
| FGF18 | CD16 | FAP | NCAM | PAX5 |
| FGF21 | CD19 | Tenascin | Prostase | OY-TES1 |
| VEGF-A | CD20 | PDL-1 | PAP | LCK |
| VEGF-B | CD21 | BAFF | ELF2M | AKAP-4 |
| VEGF-C | CD22 | HDAC | Ephrin B2 | SSX2 |
| VEGF-D | CD25 | ABL | IGF-1 | RAGE-1 |
| | | | receptor | |
| PIGF | CD28 | FLT3 | CAIX | human |
| | | | | telomerase |
| | | | | reverse |
| | | | | transcriptase |
| RET | CD34 | KIT | LMP2 | RU1 |
| Receptor and | | | | |
| the Eph | | | | |
| Receptor | | | | |
| Family | | | | |
| including | | | | |
| EphA1 | | | | |
| EphA2 | CD35 | MET | gplOO | RU2 |
| EphA3 | CD40 | RET | bcr-abl | intestinal |
| | | | | carboxyl esterase |
| EphA4 | CD45RA | IL-1β | tyrosinase | mut hsp70-2 |
| EphA5 | CD45RO | ALK | Fucosyl GM1 | CD79a |
| EphA6 | CD52 | RANKL | sLe | CD79b |
| EphA7 | CD56 | mTOR | GM3 | CD72 |
| EphA8 | CD62L | CTLA-4 | TGS5 | LAIR1 |
| EphA9 | CD68 | IL-6 | HMWMAA | FCAR |
| EphA10 | CD80 | IL-6R | o-acetyl-GD2 | LILRA2 |
| EphB1 | CD95 | JAK3 | Folate | CD300LF |
| | | | receptor beta | |
| EphB2. | CD117 | BRAF | TEM1/CD248 | CLEC12A |
| EphB3 | | | | |
| EphB4 | CD127 | PTCH | TEM7R | BST2 |
| EphB6 | CD133 | PIGF | CLDN6 | EMR2 |
| CXCR1 | CD137 (4-1 | ANPEP | GPRC5D | LY75 |
| | BB) | | | |
| CXCR2 | CD163 | TIMP1 | CXORF61 | GPC3 |
| CXCR3 | F4/80 | PLAUR | CD97 | FCRL5 |
| CXCR4 | IL-4Ra | PTPRJ | CD179a | IGLL1 |
| CXCR6 | Sca-1 | LTBR | ALK | a neoantigen |
| CCR1 | CTLA-4 | ANTXR1 | Polysialic | CD133 |
| | | | acid | |
| CCR2 | GITR | Folate | PLAC1 | CD15 |
| | | receptor alpha | | |
| | | (FRa) | | |
| CCR3 | GARP | ERBB2 | GloboH | CD184 |
| | | (Her2/neu) | | |
| CCR4 | LAP | EphA2 | NY-BR-1 | CD24 |
| CCR5 | granzyme B | IL-13Ra2 | UPK2 | CD56 |
| CCR6 | LFA-1 | epidermal | HAVCR1 | CD26 |
| | | growth factor | | |
| | | receptor | | |
| | | (EGFR) | | |
| CCR8 | transferrin | Mesothelin | ADRB3 | CD29 |
| | receptor | | | |
| CFTR | NKp46 | TSHR | PANX3 | CD44 |
| CIC-1 | perforin | CD19 | GPR20 | HLA-A |
| CIC-2 | CD4+ | CD123 | LY6K | HLA-B |
| CIC-4 | Th1 | CD22 | OR51E2 | HLA-C |
| CIC-5 | Th2 | CD30 | TARP | (HLA-A |
| CIC-7 | Th17 | CD171 | WT1 | B |
| CIC-Ka | Th40 | CS-1 | NY-ESO-1 | C) CD49f |
| CIC-Kb | Th22 | CLL-1 | LAGE-la | CD151 CD340 |
| Bestrophins | Th9 | CD33 | MAGE-A1 | CD200 |
| TMEM16A | Tfh | EGFRvIII | legumain | tkrA |
| GABA | Canonical | GD2 | HPV E6 | trkB |
| receptor | Treg. FoxP3+ | | | |
| glycin | Tr1 | GD3 | E7 | or trkC |
| receptor | | | | |
| ABC | Th3 | BCMA | ETV6-AML | TAG-72 |
| transporters | | | | |
| NAV1.1 | Treg17 | MUC16 | sperm protein | Tn Ag |
| | | (CA125) | 17 | |
| NAV1.2 | T_{RE}G | L1CAM | XAGE1 | p53 |
| NAV1.3 | CDCP1 | LeY | Tie 2 | Mucins |
| NAV1.4 | NT5E | MSLN | MAD-CT-1 | Claudin 18.2 |
| | | | | (CLDN18.2) |
| NAV1.5 | EpCAM | IL13Ra1 | MAD-CT-2 | |
| CEA | gpA33 | Fos-related | L1-CAM | |
| | | antigen 1 | | |

In some embodiments, the antigen binding domain targets an antigen characteristic of a T-cell. In some embodiments, the antigen characteristic of a T-cell is selected from an exemplary T cell antigen listed in Table C2, or an antigenic fragment thereof.

TABLE C2
Exemplary T-cell Antigens

| | | | | | |
|---|---|---|---|---|---|
| a cell surface receptor | CALM1 | HLA-DRB1 | MAP2K6 (MKK6) | NFKBIA | RAF1 |
| a membrane transport | CD3D | HLA-DRB3 | MAP2K7 (MKK7) | NRAS | RELA |
| protein | (CD3δ) | | | | |
| an active or passive | CD3E | HLA-DRB4 | MAP3K1 | PAK1 | SDF1 |
| transport protein | (CD3ε) | | (MEKK1) | | |
| an ion channel protein | CD3G | HLA-DRB5 | MAP3K3 | PAK2 | SHP2 |
| | (CD3γ) | | | | |
| a pore-forming protein | CD4 | HRAS | MAP3K4 | PAK3 | SLP76 |
| a transmembrane | CD8 | IKBKA | MAP3K5 | PAK4 | SOS |
| receptor | | (CHUK) | | | |
| a membrane enzyme | CD28 | IKBKB | MAP3K8 | PIK3C2B | SRC |
| a cell adhesion protein | CD45 | IKBKE | MAP3K14 (NIK) | PIK3C3 | TBK1 |
| characteristic of a T- | | | | (VPS34) | |
| cell | | | | | |
| a G protein-coupled | CD80 | IKBKG | MAPK8 (JNK1) | PIK3CA | TCRA |
| receptor | (B7-1) | (NEMO) | | | |
| receptor tyrosine | CD86 | IL2 | MAPK9 (JNK2) | PIK3CB | TEC |
| kinase | (B7-2) | | | | |
| tyrosine kinase | CD247 | ITPR1 | MAPK10 (JNK3) | PIK3CD | TRAF6 |
| associated receptor | (CD3ζ) | | | | |
| receptor-like tyrosine | CTLA4 | ITK | MAPK11 (p38β) | PIK3R1 | VAV1 |
| phosphatase | (CD152) | | | | |
| receptor | ELK1 | JUN | MAPK12 (p38γ) | PKCA | VAV2 |
| serine/threonine kinase | | | | | |
| receptor guanylyl | ERK1 | KRAS2 | MAPK13 (p38δ) | PKCB | ZAP70 |
| cyclase | (MAPK3) | | | | |
| histidine kinase | ERK2 | LAT | MAPK14 (p38α) | PKCM | |
| associated receptor | | | | | |
| AKT1 | FOS | LCK | NCK | PKCQ | |
| AKT2 | FYN | MAP2K1 | NFAT1 | PLCY1 | |
| | | (MEK1) | | | |
| AKT3 | GRAP2 | MAP2K2 | NFAT2 | PRF1 | |
| | (GADS) | (MEK2) | | (Perforin) | |
| ATF2 | GRB2 | MAP2K3 | NFKB1 | PTEN | |
| | | (MKK3) | | | |
| BCL10 | HLA- | MAP2K4 | NFKB2 | RAC1 | |
| | DRA | (MKK4) | | | |

In some embodiments, the antigen binding domain targets an antigen characteristic of an autoimmune or inflammatory disorder. In some embodiments, the autoimmune or inflammatory disorder is selected from chronic graft-vs-host disease (GVHD), lupus, arthritis, immune complex glomerulonephritis, goodpasture, uveitis, hepatitis, systemic sclerosis or scleroderma, type I diabetes, multiple sclerosis, cold agglutinin disease, Pemphigus vulgaris, Grave's disease, autoimmune hemolytic anemia, Hemophilia A, Primary Sjogren's Syndrome, thrombotic thrombocytopenia purrpura, neuromyelits optica, Evan's syndrome, IgM mediated neuropathy, cyroglobulinemia, dermatomyositis, idiopathic thrombocytopenia, ankylosing spondylitis, bullous pemphigoid, acquired angioedema, chronic urticarial, antiphospholipid demyelinating polyneuropathy, and autoimmune thrombocytopenia or neutropenia or pure red cell aplasias, while exemplary non-limiting examples of alloimmune diseases include allosensitization (see, for example, Blazar et al., 2015, Am. J. Transplant, 15(4):931-41) or xenosensitization from hematopoietic or solid organ transplantation, blood transfusions, pregnancy with fetal allosensitization, neonatal alloimmune thrombocytopenia, hemolytic disease of the newborn, sensitization to foreign antigens such as can occur with replacement of inherited or acquired deficiency disorders treated with enzyme or protein replacement therapy, blood products, and gene therapy. In some embodiments, the antigen characteristic of an autoimmune or inflammatory disorder is selected from an exemplary antigen listed in Table C3, or an antigenic fragment thereof. In some embodiments, the antigen binding domain targets citrullinated vimentin (e.g., associated with rheumatoid arthritis). In some embodiments, the antigen binding domain targets a human leukocyte antigen (HLA) (e.g., to induce transplant tolerance).

TABLE C3
Exemplary Autoimmune or Inflammatory Disorder Antigens

| | | | |
|---|---|---|---|
| a cell surface | a G protein- | receptor-like | histidine kinase |
| receptor | coupled | tyrosine | associated |
| | receptor | phosphatase | receptor |
| an ion channel- | receptor tyrosine | receptor serine/ | citrullinated |
| linked receptor | kinase | threonine kinase | vimentin |
| an enzyme- | tyrosine kinase | receptor guanylyl | Human |
| linked | associated | cyclase | leukocyte |
| receptor | receptor | | antigen (HLA) |

In some embodiments, a CAR antigen binding domain binds to a ligand expressed on B cells, plasma cells, plasmablasts. In some embodiments, the antigen expressed on B cells, plasma cells, or plasmablasts is selected from an exemplary antigen listed in Table C4, or an antigenic fragment thereof. In some embodiments, the B cell antigen is BCMA. In some embodiments, the B cell antigen is GPRC5D. In some embodiments, the B cell antigen is CD20. In some embodiments, a CAR that binds to an antigen listed in Table C4 is utilized to deplete B cells (e.g., autoreactive B cells producing autoantibodies) to induce immune tolerance.

TABLE C4
Exemplary B cell, Plasma Cell, and Plasmablast Antigens

| | | | | |
|---|---|---|---|---|
| CD10 | CD24 | CD138 | TNF | LFA-1 |
| CD19 | CD27 | CD319 | interferon | CD3 gamma |
| | | | receptors | |
| CD20 | CD38 | BCMA | GM-CSF | CD5 |
| CD22 | CD45R | CD28 | ZAP-70 | CD2 |
| GPRC5D | | | | |

In some embodiments, the antigen binding domain targets an antigen characteristic of an infectious disease. In some embodiments, wherein the infectious disease is selected from HIV, hepatitis B virus, hepatitis C virus, Human herpes virus, Human herpes virus 8 (HHV-8, Kaposi sarcoma-associated herpes virus (KSHV)), Human T-lymphotrophic virus-1 (HTLV-1), Merkel cell polyomavirus (MCV), Simian virus 40 (SV40), Epstein-Barr virus, CMV, human papillomavirus. In some embodiments, the antigen characteristic of an infectious disease is selected from an exemplary antigen listed in Table C5, or an antigenic fragment thereof.

TABLE C5
Exemplary Infectious Disease Antigens

| | |
|---|---|
| a cell surface receptor | receptor serine/threonine kinase |
| an ion channel-linked receptor | receptor guanylyl cyclase |
| an enzyme-linked receptor | histidine kinase associated receptor |
| a G protein-coupled receptor | HIV Env |
| receptor tyrosine kinase | gpl20 |
| tyrosine kinase associated receptor | CD4-induced epitope on HIV-1 |
| | Env. |
| receptor-like tyrosine phosphatase | |

Optionally, in some embodiments, the antigen binding domain further comprises a signal peptide. An amino acid sequence of an exemplary signal peptide is MALPVTALLLPLALLLHAARP (SEQ ID NO: 15542), which may be encoded by an exemplary nucleic acid sequence of

(SEQ ID NO: 15543)
ATGGCTCTGCCGGTGACCGCCCTGCTTCTGCCTCTTGCCCTGCTCTTGC
ATGCCGCTCGCCCG
or
(SEQ ID NO: 15544)
ATGGCTCTGCCCGTCACCGCTCTGCTGCTGCCTCTGGCTCTGCTGCTGC
ACGCTGCTCGCCCT.

### Transmembrane Domain

In some embodiments, the CAR transmembrane domain comprises at least a transmembrane region of an exemplary transmembrane domain listed in Table C6, or a functional fragment thereof.

TABLE C6
Exemplary Transmembrane Domains

| | | | | |
|---|---|---|---|---|
| alpha chain of the | CD22 | 4- | TCRα | CD80 |
| T cell receptor | | 1BB/CD137 | | |
| beta chain of the | CD33 | CD28 | TCRβ | CD86 |
| T cell receptor | | | | |
| zeta chain of the | CD37 | CD34 | TCRζ | CD40 |
| T-cell receptor | | | | |
| CD28 | CD64 | CD4 | CD32 | CD40L/CD154 |
| CD3 epsilon | CD80 | FcεRIγ | CD64 | VEGFR2 |
| CD45 | CD86 | CD16 | CD64 | FAS |
| CD4 | CD134 | OX40/CD134 | CD45 | FGFR2B |
| CD5 | CD137 | CD3ζ | CD5 | |
| CD8 | CD154 | CD3ε | CD9 | |
| CD9 | CD8α | CD3γ | CD22 | |
| CD16 | CD8β | CD3δ | CD37 | |

In some embodiments, the transmembrane domain is a CD8 transmembrane domain. In some embodiments, the CD8 transmembrane domain has an amino acid sequence of a CD8 transmembrane domain listed in Table C6A, or sequence having at least 75%, 8000, 8500, 9000, 95%, 960%, 9700, 980%, or 9900 identity thereto. In some embodiments, the CD8 transmembrane domain is encoded by a nucleic acid sequence of a CD8 transmembrane domain listed in Table C6A, or sequence having at least 75%, 80%, 85%, 90%, 95%, 96%, 97%, 98%, or 99% identity thereto.

TABLE C6A
Sequences of Exemplary Transmembrane Domains

| | | SEQ ID |
|---|---|---|
| Name | Sequence | NO: |
| CD8 | IYIWAPLAGTCGVLLLSLVITLYC | 1102 |
| transmembrane | | |
| (amino acid) | | |
| CD8 | ATCTACATCTGGGCGCCCTTGGCCGGGACTTGTGGGG | 15474 |
| transmembrane | TCCTTCTCCTGTCACTGGTTATCACCCTTTACTGC | |
| (nucleic acid) | | |
| CD8 | ATCTACATTTGGGCCCCTCTGGCTGGTACTTGCGGGG | 15475 |
| transmembrane | TCCTGCTGCTTTCACTCGTGATCACTCTTTACTGT | |
| (nucleic acid) | | |
| CD8 | IYIWAPLAGTCGVLLLSLVITLYCNHRN | 15476 |
| transmembrane | | |
| (amino acid) | | |
| CD8 | ATCTACATCTGGGCCCCTCTGGCCGGCACCTGTGGCG | 15477 |
| transmembrane | TGCTGCTGCTGAGCCTGGTGATCACCCTGTACTGCAA | |
| (nucleic acid) | CCACCGGAAC | |
| CD8α | IYIWAPLAGTCGVLLLSLVITYC | 15554 |
| transmembrane | | |
| domain (amino | | |
| acid) | | |

### Intracellular Signaling Domains

In some embodiments, the CAR comprises at least one signaling domain selected from one or more intracellular signaling domains listed in Table C7, or a functional fragment thereof. In some embodiments, the CAR comprises a first intracellular signaling domain and a second intracellular signaling domain. In some embodiments, the first intracellular signaling domain mediates downstream signaling during T-cell activation. In some embodiments, the second intracellular signaling domain is a costimulatory domain.

TABLE C7
Exemplary Intracellular Signaling Domains

| | | | |
|---|---|---|---|
| B7-1/CD80 | 4-1BB Ligand/TNFSF9 | RELT/TNFRSF19L | CD7 |
| B7-2/CD86 | BAFF/BLyS/TNFSF13B | TACI/TNFRSF13B | CD53 |
| B7-H1/PD-L1 | BAFF R/TNFRSF13C | TL1A/TNFSF15 | CD82/Kai-1 |
| B7-H2 | CD27/TNFRSF7 | TNF-alpha | CD90/Thy 1 |
| B7-H3 | CD27 Ligand/TNFSF7 | TNF RII/TNFRSF1B | CD96 |
| B7-H4 | CD30/TNFRSF8 | 2B4/CD244/SLAMF4 | CD160 |
| B7-H6 | CD30 Ligand/TNFSF8 | BLAME/SLAMF8 | CD200 |
| B7-H7 | CD40/TNFRSF5 | CD2 | CD300a/LMIR1 |
| BTLA/CD272 | CD40/TNFSF5 | CD2F-10/SLAMF9 | HLA Class I |
| CD28 | CD40 Ligand/TNFSF5 | CD48/SLAMF2 | HLA-DR |
| CTLA-4 | DR3/TNFRSF25 | CD58/LFA-3 | Ikaros |
| Gi24/VISTA/B7-H5 | GITR/TNFRSF18 | CD84/SLAMF5 | Integrin alpha |
| | | | 4/CD49d |
| ICOS/CD278 | GITR Ligand/TNFSF18 | CD229/SLAMF3 | Integrin alpha 4 beta |
| | | | 1 |
| PD-1 | HVEM/TNFRSF14 | CRACC/SLAMF7 | Integrin alpha 4 beta |
| | | | 7/LPAM-1 |
| PD-L2/B7-DC | LIGHT/TNFSF14 | NTB-A/SLAMF6 | LAG-3 |
| PDCD6 | Lymphotoxin- | SLAM/CD150 | TCL1A |
| | alpha/TNF-beta | | |
| 4- | OX40/TNFRSF4 | CD2 | TCL1B |
| 1BB/TNFSF9/CD137 | | | |
| CRTAM | CD30 | TIM-1/KIM- | CD2 |
| | | 1/HAVCR | |
| DAP12 | CD40 | TIM-4 | CD7 |
| Dectin-1/CLEC7A | PD-1 | TSLP | LIGHT |
| DPPIV/CD26 | ICOS | TSLP R | NKG2C |
| EphB6 | lymphocyte function- | lymphocyte function | B7-H3 |
| | associated antigen-1 | associated antigen-1 | |
| | (LFA-1) | (LFA-1) | |
| NKG2C | a ligand that specifically | a CD3 zeta domain | an immunoreceptor |
| | binds with CD83 | | tyrosine-based |
| | | | activation motif |
| | | | (ITAM) |
| CD27 | CD28 | 4-1BB | CD134/OX40 |

In some embodiments, the CAR comprises a (i) a CD3 zeta domain, or an immunoreceptor tyrosine-based activation motif (ITAM), or functional variant thereof, (ii) a CD28 domain or functional variant thereof, and (iii) a 4-1BB domain, or a CD134 domain, or functional variant thereof. In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof, (ii) a CD28 domain, or a 4-1BB domain, or functional variant thereof, and/or (iii) a 4-1BB domain, or a CD134 domain, or functional variant thereof. In some embodiments, the CAR comprises a (i) a CD3 zeta domain, or an immunoreceptor tyrosine-based activation motif (ITAM), or functional variant thereof, (ii) a CD28 domain or functional variant thereof, (iii) a 4-1BB domain, or a CD 134 domain, or functional variant thereof, and (iv) a cytokine or costimulatory ligand transgene.

In some embodiments, the CAR comprises a CD28 co-stimulatory domain.

In some embodiments, the CAR comprises a CD3z signaling domain.

In some embodiments, intracellular signaling domain comprises an intracellular signaling domain listed in Table C7A, or sequence having at least 75, 80, 85, 900, 95%, 960, 970, 98%, or 99% identity thereto. In some embodiments, intracellular signaling domain is encoded by a nucleic acid sequence of an intracellular signaling domain listed in Table C7A, or sequence having at least 75%, 80%, 85%, 90%, 95%, 96%, 97%, 98%, or 99%, identity thereto.

TABLE C7A
Sequences of Exemplary Intracellular Signaling Domains

| | | SEQ ID |
|---|---|---|
| Name | Sequence | NO: |
| CD28 signaling | RSKRSRLLHSDYMNMTPRRPGPTRKHYQPYAPPRDFAA | 1103 |
| domain (amino | YRS | |
| acid) | | |
| CD28 signaling | AGGAGTAAGAGGAGCAGGCTCCTGCACAGTGACTAC | 15446 |
| domain (nucleic | ATGAACATGACTCCCCGCCGCCCCGGGCCCACCCGCA | |
| acid) | AGCATTACCAGCCCTATGCCCCACCACGCGACTTCGC | |
| | AGCCTATCGCTCC | |
| CD3z signaling | RVKFSRSADAPAYQQGQNQLYNELNLGRREEYDVLDK | 1104 |
| domain (amino | RRGRDPEMGGKPRRKNPQEGLYNELQKDKMAEAYSEI | |
| acid) | GMKGERRRGKGHDGLYQGLSTATKDTYDALHMQALP | |
| | PR | |
| CD3z signaling | AGAGTGAAGTTCAGCAGATCCGCCGACGCCCCTGCCT | 15478 |
| domain (nucleic | ACCAGCAGGGACAGAACCAGCTGTACAACGAGCTGA | |
| acid) | ACCTGGGCAGACGGGAAGAGTACGACGTGCTGGACA | |
| | AGCGGAGAGGCCGGGACCCCGAGATGGGCGGAAAG | |
| | CCCAGACGGAAGAACCCCCAGGAAGGCCTGTATAAC | |
| | GAACTGCAGAAAGACAAGATGGCCGAGGCCTACAGC | |
| | GAGATCGGCATGAAGGGCGAGCGGAGGCGCGGCAA | |
| | GGGCCACGATGGCCTGTACCAGGGCCTGAGCACCGC | |
| | CACCAAGGACACCTACGACGCCCTGCACATGCAGGC | |
| | CCTGCCCCCCAGA | |
| CD3z signaling | AGAGTGAAGTTCAGCAGGAGCGCAGACGCCCCCGCG | 15479 |
| domain (nucleic | TACCAGCAGGGCCAGAACCAGCTCTATAACGAGCTC | |
| acid) | AATCTAGGACGAAGAGAGGAGTACGATGTTTTGGAC | |
| | AAGAGACGTGGCCGGGACCCTGAGATGGGGGGAAAG | |
| | CCGAGAAGGAAGAACCCTCAGGAAGGCCTGTACAAT | |
| | GAACTGCAGAAAGATAAGATGGCGGAGGCCTACAGT | |
| | GAGATTGGGATGAAAGGCGAGCGCCGGAGGGGCAA | |
| | GGGGCACGATGGCCTTTACCAGGGTCTCAGTACAGCC | |
| | ACCAAGGACACCTACGACGCCCTTCACATGCAGGCC | |
| | CTGCCCCCTCGC | |
| 4-1BB signaling | KRGRKKLLYIFKQPFMRPVQTTQEEDGCSCRFPEEEEGG | 15480 |
| domain (amino | CEL | |
| acid) | | |
| 4-1BB signaling | AAACGGGGCAGAAAGAAACTCCTGTATATATTCAAA | 15481 |
| domain (nucleic | | |
| acid) | CAACCATTTATGAGACCAGTACAAACTACTCAAGAG | |
| | GAAGATGGCTGTAGCTGCCGATTTCCAGAAGAAGAA | |
| | GAAGGAGGATGTGAACTG | |

In some embodiments, the CAR further comprises one or more spacers, e.g., wherein the spacer is a first spacer between the antigen binding domain and the transmembrane domain. In some embodiments, the first spacer includes at least a portion of an immunoglobulin constant region or variant or modified version thereof. In some embodiments, the spacer is a second spacer between the transmembrane domain and a signaling domain. In some embodiments, the second spacer is an oligopeptide, e.g., wherein the oligopeptide comprises glycine-serine doublets. In some embodiments, the CAR further comprises a hinge domain. In some embodiments, the hinge domain is a CD8 hinge domain. In some embodiments, the CD8 hinge domain has an amino acid sequence of a CD8 hinge domain in Table C8, or sequence having at least 75%, 80%, 85%, 90%, 95%, 96%, 97%, 98%, or 99% identity thereto. In some embodiments, the CD38 hinge domain is encoded by a nucleic sequence listed in Table C8, or sequence having at least 75%, 80%, 85%, 90%, 95%, 96%, 97%, 98%, or 99% identity thereto.

TABLE C8
Sequence of Exemplary Hinge Domains

| | | SEQ ID |
|---|---|---|
| Name | Sequence | NO: |
| CD8 Hinge | TTTPAPRPPTPAPTIASQPLSLRPEACRPAAGGAVHTRGL | 1101 |
| (amino acid) | DFACD | |
| CD8 Hinge | ACCACGACGCCAGCGCCGCGACCACCAACACCGGCG | 15482 |
| (nucleic acid) | CCCACCATCGCGTCGCAGCCCCTGTCCCTGCGCCCAG | |
| | AGGCGTGCCGGCCAGCGGCGGGGGGCGCAGTGCACA | |
| | CGAGGGGGCTGGACTTCGCCTGTGAT | |
| CD8 Hinge | ACCACTACCCCAGCACCGAGGCCACCCACCCCGGCTC | 15483 |
| (nucleic acid) | CTACCATCGCCTCCCAGCCTCTGTCCCTGCGTCCGGA | |
| | GGCATGTAGACCCGCAGCTGGTGGGGCCGTGCATAC | |
| | CCGGGGTCTTGACTTCGCCTGCGAT | |
| CD8 Hinge | FVPVFLPAKPTTTPAPRPPTPAPTIASQPLSLRPEACRPAA | 15484 |
| (amino acid) | GGAVHTRGLDFACD | |
| CD8 Hinge | TTCGTGCCCGTGTTCCTGCCCGCCAAACCTACCACCA | 15485 |
| (nucleic acid) | CCCCTGCCCCTAGACCTCCCACCCCAGCCCCAACAAT | |
| | CGCCAGCCAGCCTCTGTCTCTGCGGCCCGAAGCCTGT | |
| | AGACCTGCTGCCGGCGGAGCCGTGCACACCAGAGGC | |
| | CTGGACTTCGCCTGCGAC | |

In some embodiments, the CAR comprises a sequence of a CAR listed in Table C9, or sequence having at least 75%, 80%, 85%, 90%, 95%, 96%, 97%, 98%, or 99% identity thereto. In some embodiments, the CAR is encoded by a nucleic acid sequence listed in Table C9, or sequence having at least 75%, 80%, 85%, 90%, 95%, 96%, 97%, 98%, or 99%, identity thereto.

TABLE C9
Sequences of Exemplary CAR Molecules

| | | |
|---|---|---|
| | | SEQ ID |
| Description | Sequence | NO |

BCMA CAR 1

| | | |
|---|---|---|
| BCMA CAR1 | DIQMTQSPSSLSASVGDRVTITCRASQSISSYLNWYQQKPGK | 15486 |
| VL | APKLLIYAASSLQSGVPSRFSGSGSGTDFTLTISSLQPEDFATY | |
| (amino acid) | YCQQKYDLLTFGGGTKVEIK | |
| BCMA CAR1 | GACATCCAGATGACCCAGTCTCCATCCTCCCTGTCTGCATC | 15487 |
| VL | TGTAGGAGACAGAGTCACCATCACTTGCCGGGCAAGTCAG | |
| (nucleic acid) | AGCATTAGCAGCTATTTAAATTGGTATCAGCAGAAACCAG | |
| | GGAAAGCCCCTAAGCTCCTGATCTATGCTGCATCCAGTTT | |
| | GCAAAGTGGGGTCCCATCAAGGTTCAGTGGCAGTGGATCT | |
| | GGGACAGATTTCACTCTCACCATCAGCAGTCTGCAACCTG | |
| | AAGATTTTGCAACTTACTACTGTCAGCAAAAATACGACCT | |
| | CCTCACTTTTGGCGGAGGGACCAAGGTTGAGATCAAA | |
| BCMA CAR1 | QLQLQESGPGLVKPSETLSLTCTVSGGSISSSSYYWGWIRQPP | 15488 |
| VH | GKGLEWIGSISYSGSTYYNPSLKSRVTISVDTSKNQFSLKLSS | |
| (amino acid) | VTAADTAVYYCARDRGDTILDVWGQGTMVTVSS | |
| BCMA CAR1 | CAGCTGCAGCTGCAGGAGTCGGGCCCAGGACTGGTGAAG | 15489 |
| VH | CCTTCGGAGACCCTGTCCCTCACCTGCACTGTCTCTGGTGG | |
| (nucleic acid) | CTCCATCAGCAGTAGTAGTTACTACTGGGGCTGGATCCGC | |
| | CAGCCCCCAGGGAAGGGGCTGGAGTGGATTGGGAGTATC | |
| | TCCTATAGTGGGAGCACCTACTACAACCCGTCCCTCAAGA | |
| | GTCGAGTCACCATATCCGTAGACACGTCCAAGAACCAGTT | |
| | CTCCCTGAAGCTGAGTTCTGTGACCGCCGCAGACACGGCG | |
| | GTGTACTACTGCGCCAGAGATCGTGGAGACACCATACTAG | |
| | ACGTATGGGGTCAGGGTACAATGGTCACCGTCAGCTCA | |
| BCMA CAR1 | DIQMTQSPSSLSASVGDRVTITCRASQSISSYLNWYQQKPGK | 15490 |
| Full CAR (no | APKLLIYAASSLQSGVPSRFSGSGSGTDFTLTISSLQPEDFATY | |
| signal peptide) | YCQQKYDLLTFGGGTKVEIKGSTSGSGKPGSGEGSTKGQLQ | |
| (amino acid) | LQESGPGLVKPSETLSLTCTVSGGSISSSSYYWGWIRQPPGKG | |
| | LEWIGSISYSGSTYYNPSLKSRVTISVDTSKNQFSLKLSSVTA | |
| | ADTAVYYCARDRGDTILDVWGQGTMVTVSSFVPVFLPAKPT | |
| | TTPAPRPPTPAPTIASQPLSLRPEACRPAAGGAVHTRGLDFAC | |
| | DIYIWAPLAGTCGVLLLSLVITLYCNHRNKRGRKKLLYIFKQ | |
| | PFMRPVQTTQEEDGCSCRFPEEEEGGCELRVKFSRSADAPAY | |
| | QQGQNQLYNELNLGRREEYDVLDKRRGRDPEMGGKPRRKN | |
| | PQEGLYNELQKDKMAEAYSEIGMKGERRRGKGHDGLYQGL | |
| | STATKDTYDALHMQALPPR | |
| BCMA CAR1 | GACATCCAGATGACCCAGTCTCCATCCTCCCTGTCTGCATC | 15491 |
| Full CAR (no | TGTAGGAGACAGAGTCACCATCACTTGCCGGGCAAGTCAG | |
| signal peptide) | AGCATTAGCAGCTATTTAAATTGGTATCAGCAGAAACCAG | |
| (nucleic acid) | GGAAAGCCCCTAAGCTCCTGATCTATGCTGCATCCAGTTT | |
| | GCAAAGTGGGGTCCCATCAAGGTTCAGTGGCAGTGGATCT | |
| | GGGACAGATTTCACTCTCACCATCAGCAGTCTGCAACCTG | |
| | AAGATTTTGCAACTTACTACTGTCAGCAAAAATACGACCT | |
| | CCTCACTTTTGGCGGAGGGACCAAGGTTGAGATCAAAGGC | |
| | AGCACCAGCGGCTCCGGCAAGCCTGGCTCTGGCGAGGGC | |
| | AGCACAAAGGGACAGCTGCAGCTGCAGGAGTCGGGCCCA | |
| | GGACTGGTGAAGCCTTCGGAGACCCTGTCCCTCACCTGCA | |
| | CTGTCTCTGGTGGCTCCATCAGCAGTAGTAGTTACTACTG | |
| | GGGCTGGATCCGCCAGCCCCCAGGGAAGGGGCTGGAGTG | |
| | GATTGGGAGTATCTCCTATAGTGGGAGCACCTACTACAAC | |
| | CCGTCCCTCAAGAGTCGAGTCACCATATCCGTAGACACGT | |
| | CCAAGAACCAGTTCTCCCTGAAGCTGAGTTCTGTGACCGC | |
| | CGCAGACACGGCGGTGTACTACTGCGCCAGAGATCGTGG | |
| | AGACACCATACTAGACGTATGGGGTCAGGGTACAATGGTC | |
| | ACCGTCAGCTCATTCGTGCCCGTGTTCCTGCCCGCCAAAC | |
| | CTACCACCACCCCTGCCCCTAGACCTCCCACCCCAGCCCC | |
| | AACAATCGCCAGCCAGCCTCTGTCTCTGCGGCCCGAAGCC | |
| | TGTAGACCTGCTGCCGGCGGAGCCGTGCACACCAGAGGCC | |
| | TGGACTTCGCCTGCGACATCTACATCTGGGCCCCTCTGGC | |
| | CGGCACCTGTGGCGTGCTGCTGCTGAGCCTGGTGATCACC | |
| | CTGTACTGCAACCACCGGAACAAACGGGGCAGAAAGAAA | |
| | CTCCTGTATATATTCAAACAACCATTTATGAGACCAGTAC | |
| | AAACTACTCAAGAGGAAGATGGCTGTAGCTGCCGATTTCC | |
| | AGAAGAAGAAGAAGGAGGATGTGAACTGAGAGTGAAGTT | |
| | CAGCAGATCCGCCGACGCCCCTGCCTACCAGCAGGGACA | |
| | GAACCAGCTGTACAACGAGCTGAACCTGGGCAGACGGGA | |
| | AGAGTACGACGTGCTGGACAAGCGGAGAGGCCGGGACCC | |
| | CGAGATGGGCGGAAAGCCCAGACGGAAGAACCCCCAGGA | |
| | AGGCCTGTATAACGAACTGCAGAAAGACAAGATGGCCGA | |
| | GGCCTACAGCGAGATCGGCATGAAGGGCGAGCGGAGGCG | |
| | CGGCAAGGGCCACGATGGCCTGTACCAGGGCCTGAGCAC | |
| | CGCCACCAAGGACACCTACGACGCCCTGCACATGCAGGCC | |
| | CTGCCCCCCAGA | |
| BCMA CAR1 | MALPVTALLLPLALLLHAARPDIQMTQSPSSLSASVGDRVTIT | 15492 |
| Full CAR (with | CRASQSISSYLNWYQQKPGKAPKLLIYAASSLQSGVPSRFSGS | |
| signal peptide) | GSGTDFTLTISSLQPEDFATYYCQQKYDLLTFGGGTKVEIKGS | |
| (amino acid) | TSGSGKPGSGEGSTKGQLQLQESGPGLVKPSETLSLTCTVSG | |
| | GSISSSSYYWGWIRQPPGKGLEWIGSISYSGSTYYNPSLKSRV | |
| | TISVDTSKNQFSLKLSSVTAADTAVYYCARDRGDTILDVWG | |
| | QGTMVTVSSFVPVFLPAKPTTTPAPRPPTPAPTIASQPLSLRPE | |
| | ACRPAAGGAVHTRGLDFACDIYIWAPLAGTCGVLLLSLVITL | |
| | YCNHRNKRGRKKLLYIFKQPFMRPVQTTQEEDGCSCRFPEEE | |
| | EGGCELRVKFSRSADAPAYQQGQNQLYNELNLGRREEYDVL | |
| | DKRRGRDPEMGGKPRRKNPQEGLYNELQKDKMAEAYSEIG | |
| | MKGERRRGKGHDGLYQGLSTATKDTYDALHMQALPPR | |
| BCMA CAR1 | ATGGCTCTGCCGGTGACCGCCCTGCTTCTGCCTCTTGCCCT | 15493 |
| Full CAR (with | GCTCTTGCATGCCGCTCGCCCGGACATCCAGATGACCCAG | |
| signal peptide) | TCTCCATCCTCCCTGTCTGCATCTGTAGGAGACAGAGTCA | |
| (nucleic acid) | CCATCACTTGCCGGGCAAGTCAGAGCATTAGCAGCTATTT | |
| | AAATTGGTATCAGCAGAAACCAGGGAAAGCCCCTAAGCT | |
| | CCTGATCTATGCTGCATCCAGTTTGCAAAGTGGGGTCCCA | |
| | TCAAGGTTCAGTGGCAGTGGATCTGGGACAGATTTCACTC | |
| | TCACCATCAGCAGTCTGCAACCTGAAGATTTTGCAACTTA | |
| | CTACTGTCAGCAAAAATACGACCTCCTCACTTTTGGCGGA | |
| | GGGACCAAGGTTGAGATCAAAGGCAGCACCAGCGGCTCC | |
| | GGCAAGCCTGGCTCTGGCGAGGGCAGCACAAAGGGACAG | |
| | CTGCAGCTGCAGGAGTCGGGCCCAGGACTGGTGAAGCCTT | |
| | CGGAGACCCTGTCCCTCACCTGCACTGTCTCTGGTGGCTCC | |
| | ATCAGCAGTAGTAGTTACTACTGGGGCTGGATCCGCCAGC | |
| | CCCCAGGGAAGGGGCTGGAGTGGATTGGGAGTATCTCCTA | |
| | TAGTGGGAGCACCTACTACAACCCGTCCCTCAAGAGTCGA | |
| | GTCACCATATCCGTAGACACGTCCAAGAACCAGTTCTCCC | |
| | TGAAGCTGAGTTCTGTGACCGCCGCAGACACGGCGGTGTA | |
| | CTACTGCGCCAGAGATCGTGGAGACACCATACTAGACGTA | |
| | TGGGGTCAGGGTACAATGGTCACCGTCAGCTCATTCGTGC | |
| | CCGTGTTCCTGCCCGCCAAACCTACCACCACCCCTGCCCCT | |
| | AGACCTCCCACCCCAGCCCCAACAATCGCCAGCCAGCCTC | |
| | TGTCTCTGCGGCCCGAAGCCTGTAGACCTGCTGCCGGCGG | |
| | AGCCGTGCACACCAGAGGCCTGGACTTCGCCTGCGACATC | |
| | TACATCTGGGCCCCTCTGGCCGGCACCTGTGGCGTGCTGC | |
| | TGCTGAGCCTGGTGATCACCCTGTACTGCAACCACCGGAA | |
| | CAAACGGGGCAGAAAGAAACTCCTGTATATATTCAAACA | |
| | ACCATTTATGAGACCAGTACAAACTACTCAAGAGGAAGAT | |
| | GGCTGTAGCTGCCGATTTCCAGAAGAAGAAGAAGGAGGA | |
| | TGTGAACTGAGAGTGAAGTTCAGCAGATCCGCCGACGCCC | |
| | CTGCCTACCAGCAGGGACAGAACCAGCTGTACAACGAGC | |
| | TGAACCTGGGCAGACGGGAAGAGTACGACGTGCTGGACA | |
| | AGCGGAGAGGCCGGGACCCCGAGATGGGCGGAAAGCCCA | |
| | GACGGAAGAACCCCCAGGAAGGCCTGTATAACGAACTGC | |
| | AGAAAGACAAGATGGCCGAGGCCTACAGCGAGATCGGCA | |
| | TGAAGGGCGAGCGGAGGCGCGGCAAGGGCCACGATGGCC | |
| | TGTACCAGGGCCTGAGCACCGCCACCAAGGACACCTACG | |
| | ACGCCCTGCACATGCAGGCCCTGCCCCCCAGA | |

BCMA CAR2

| | | |
|---|---|---|
| BCMA CAR2 | QVKLEESGGGLVQAGRSLRLSCAASEHTFSSHVMGWFRQAP | 15494 |
| VHH1 (amino | GKERESVAVIGWRDISTSYADSVKGRFTISRDNAKKTLYLQM | |
| acid) | NSLKPEDTAVYYCAARRIDAADFDSWGQGTQVTVSS | |
| BCMA CAR2 | CAGGTCAAACTGGAAGAATCTGGCGGAGGCCTGGTGCAGG | 15495 |
| VHH1 (nucleic | CAGGACGGAGCCTGCGCCTGAGCTGCGCAGCATCCGAGCA | |
| acid) | CACCTTCAGCTCCCACGTGATGGGCTGGTTTCGGCAGGCCC | |
| | CAGGCAAGGAGAGAGAGAGCGTGGCCGTGATCGGCTGGA | |
| | GGGACATCTCCACATCTTACGCCGATTCCGTGAAGGGCCG | |
| | GTTCACCATCAGCCGGGACAACGCCAAGAAGACACTGTAT | |
| | CTGCAGATGAACAGCCTGAAGCCCGAGGACACCGCCGTGT | |
| | ACTATTGCGCAGCAAGGAGAATCGACGCAGCAGACTTTGA | |
| | TTCCTGGGGCCAGGGCACCCAGGTGACAGTGTCTAGC | |
| BCMA CAR2 | EVQLVESGGGLVQAGGSLRLSCAASGRTFTMGWFRQAPGKE | 15496 |
| VHH2 (amino | REFVAAISLSPTLAYYAESVKGRFTISRDNAKNTVVLQMNSLK | |
| acid) | PEDTALYYCAADRKSVMSIRPDYWGQGTQVTVSS | |
| BCMA CAR2 | GAGGTGCAGCTGGTGGAGAGCGGAGGCGGCCTGGTGCAGG | 15497 |
| VHH2 (nucleic | CCGGAGGCTCTCTGAGGCTGAGCTGTGCAGCATCCGGAAG | |
| acid) | AACCTTCACAATGGGCTGGTTTAGGCAGGCACCAGGAAAG | |
| | GAGAGGGAGTTCGTGGCAGCAATCAGCCTGTCCCCTACCC | |
| | TGGCCTACTATGCCGAGAGCGTGAAGGGCAGGTTTACCAT | |
| | CTCCCGCGATAACGCCAAGAATACAGTGGTGCTGCAGATG | |
| | AACTCCCTGAAACCTGAGGACACAGCCCTGTACTATTGTGC | |
| | CGCCGATCGGAAGAGCGTGATGAGCATTAGACCAGACTAT | |
| | TGGGGGCAGGGAACACAGGTGACCGTGAGCAGC | |
| BCMA CAR2 | QVKLEESGGGLVQAGRSLRLSCAASEHTFSSHVMGWFRQAP | 15498 |
| Full CAR (no | GKERESVAVIGWRDISTSYADSVKGRFTISRDNAKKTLYLQM | |
| signal peptide) | NSLKPEDTAVYYCAARRIDAADFDSWGQGTQVTVSSGGGGS | |
| (amino acid) | EVQLVESGGGLVQAGGSLRLSCAASGRTFTMGWFRQAPGKE | |
| | REFVAAISLSPTLAYYAESVKGRFTISRDNAKNTVVLQMNSLK | |
| | PEDTALYYCAADRKSVMSIRPDYWGQGTQVTVSSTTTPAPRP | |
| | PTPAPTIASQPLSLRPEACRPAAGGAVHTRGLDFACDIYIWAP | |
| | LAGTCGVLLLSLVITLYCKRGRKKLLYIFKQPFMRPVQTTQEE | |
| | DGCSCRFPEEEEGGCELRVKFSRSADAPAYQQGQNQLYNELN | |
| | LGRREEYDVLDKRRGRDPEMGGKPRRKNPQEGLYNELQKDK | |
| | MAEAYSEIGMKGERRRGKGHDGLYQGLSTATKDTYDALHM | |
| | QALPPR | |
| BCMA CAR2 | CAGGTCAAACTGGAAGAATCTGGCGGAGGCCTGGTGCAGG | 15499 |
| Full CAR (no | CAGGACGGAGCCTGCGCCTGAGCTGCGCAGCATCCGAGCA | |
| signal peptide) | CACCTTCAGCTCCCACGTGATGGGCTGGTTTCGGCAGGCCC | |
| (nucleic acid) | CAGGCAAGGAGAGAGAGAGCGTGGCCGTGATCGGCTGGA | |
| | GGGACATCTCCACATCTTACGCCGATTCCGTGAAGGGCCG | |
| | GTTCACCATCAGCCGGGACAACGCCAAGAAGACACTGTAT | |
| | CTGCAGATGAACAGCCTGAAGCCCGAGGACACCGCCGTGT | |
| | ACTATTGCGCAGCAAGGAGAATCGACGCAGCAGACTTTGA | |
| | TTCCTGGGGCCAGGGCACCCAGGTGACAGTGTCTAGCGGA | |
| | GGAGGAGGATCTGAGGTGCAGCTGGTGGAGAGCGGAGGC | |
| | GGCCTGGTGCAGGCCGGAGGCTCTCTGAGGCTGAGCTGTG | |
| | CAGCATCCGGAAGAACCTTCACAATGGGCTGGTTTAGGCA | |
| | GGCACCAGGAAAGGAGAGGGAGTTCGTGGCAGCAATCAG | |
| | CCTGTCCCCTACCCTGGCCTACTATGCCGAGAGCGTGAAGG | |
| | GCAGGTTTACCATCTCCCGCGATAACGCCAAGAATACAGT | |
| | GGTGCTGCAGATGAACTCCCTGAAACCTGAGGACACAGCC | |
| | CTGTACTATTGTGCCGCCGATCGGAAGAGCGTGATGAGCA | |
| | TTAGACCAGACTATTGGGGGCAGGGAACACAGGTGACCGT | |
| | GAGCAGCACCACGACGCCAGCGCCGCGACCACCAACACCG | |
| | GCGCCCACCATCGCGTCGCAGCCCCTGTCCCTGCGCCCAGA | |
| | GGCGTGCCGGCCAGCGGCGGGGGGCGCAGTGCACACGAG | |
| | GGGGCTGGACTTCGCCTGTGATATCTACATCTGGGCGCCCT | |
| | TGGCCGGGACTTGTGGGGTCCTTCTCCTGTCACTGGTTATC | |
| | ACCCTTTACTGCAAACGGGGCAGAAAGAAACTCCTGTATA | |
| | TATTCAAACAACCATTTATGAGACCAGTACAAACTACTCAA | |
| | GAGGAAGATGGCTGTAGCTGCCGATTTCCAGAAGAAGAAG | |
| | AAGGAGGATGTGAACTGAGAGTGAAGTTCAGCAGGAGCGC | |
| | AGACGCCCCCGCGTACCAGCAGGGCCAGAACCAGCTCTAT | |
| | AACGAGCTCAATCTAGGACGAAGAGAGGAGTACGATGTTT | |
| | TGGACAAGAGACGTGGCCGGGACCCTGAGATGGGGGGAA | |
| | AGCCGAGAAGGAAGAACCCTCAGGAAGGCCTGTACAATGA | |
| | ACTGCAGAAAGATAAGATGGCGGAGGCCTACAGTGAGATT | |
| | GGGATGAAAGGCGAGCGCCGGAGGGGCAAGGGGCACGAT | |
| | GGCCTTTACCAGGGTCTCAGTACAGCCACCAAGGACACCT | |
| | ACGACGCCCTTCACATGCAGGCCCTGCCCCCTCGCTAA | |
| BCMA CAR2 | MALPVTALLLPLALLLHAARPQVKLEESGGGLVQAGRSLRLS | 15500 |
| Full CAR (with | CAASEHTFSSHVMGWFRQAPGKERESVAVIGWRDISTSYADS | |
| signal peptide) | VKGRFTISRDNAKKTLYLQMNSLKPEDTAVYYCAARRIDAA | |
| (amino acid) | DFDSWGQGTQVTVSSGGGGSEVQLVESGGGLVQAGGSLRLS | |
| | CAASGRTFTMGWFRQAPGKEREFVAAISLSPTLAYYAESVKG | |
| | RFTISRDNAKNTVVLQMNSLKPEDTALYYCAADRKSVMSIRP | |
| | DYWGQGTQVTVSSTTTPAPRPPTPAPTIASQPLSLRPEACRPA | |
| | AGGAVHTRGLDFACDIYIWAPLAGTCGVLLLSLVITLYCKRG | |
| | RKKLLYIFKQPFMRPVQTTQEEDGCSCRFPEEEEGGCELRVKF | |
| | SRSADAPAYQQGQNQLYNELNLGRREEYDVLDKRRGRDPEM | |
| | GGKPRRKNPQEGLYNELQKDKMAEAYSEIGMKGERRRGKG | |
| | HDGLYQGLSTATKDTYDALHMQALPPR | |
| BCMA CAR2 | ATGGCTCTGCCCGTCACCGCTCTGCTGCTGCCTCTGGCTCT | 15501 |
| Full CAR (with | GCTGCTGCACGCTGCTCGCCCTCAGGTCAAACTGGAAGAA | |
| signal peptide) | TCTGGCGGAGGCCTGGTGCAGGCAGGACGGAGCCTGCGCC | |
| (nucleic acid) | TGAGCTGCGCAGCATCCGAGCACACCTTCAGCTCCCACGTG | |
| | ATGGGCTGGTTTCGGCAGGCCCCAGGCAAGGAGAGAGAGA | |
| | GCGTGGCCGTGATCGGCTGGAGGGACATCTCCACATCTTAC | |
| | GCCGATTCCGTGAAGGGCCGGTTCACCATCAGCCGGGACA | |
| | ACGCCAAGAAGACACTGTATCTGCAGATGAACAGCCTGAA | |
| | GCCCGAGGACACCGCCGTGTACTATTGCGCAGCAAGGAGA | |
| | ATCGACGCAGCAGACTTTGATTCCTGGGGCCAGGGCACCC | |
| | AGGTGACAGTGTCTAGCGGAGGAGGAGGATCTGAGGTGCA | |
| | GCTGGTGGAGAGCGGAGGCGGCCTGGTGCAGGCCGGAGGC | |
| | TCTCTGAGGCTGAGCTGTGCAGCATCCGGAAGAACCTTCAC | |
| | AATGGGCTGGTTTAGGCAGGCACCAGGAAAGGAGAGGGA | |
| | GTTCGTGGCAGCAATCAGCCTGTCCCCTACCCTGGCCTACT | |
| | ATGCCGAGAGCGTGAAGGGCAGGTTTACCATCTCCCGCGA | |
| | TAACGCCAAGAATACAGTGGTGCTGCAGATGAACTCCCTG | |
| | AAACCTGAGGACACAGCCCTGTACTATTGTGCCGCCGATC | |
| | GGAAGAGCGTGATGAGCATTAGACCAGACTATTGGGGGCA | |
| | GGGAACACAGGTGACCGTGAGCAGCACCACGACGCCAGCG | |
| | CCGCGACCACCAACACCGGCGCCCACCATCGCGTCGCAGC | |
| | CCCTGTCCCTGCGCCCAGAGGCGTGCCGGCCAGCGGCGGG | |
| | GGGCGCAGTGCACACGAGGGGGCTGGACTTCGCCTGTGAT | |
| | ATCTACATCTGGGCGCCCTTGGCCGGGACTTGTGGGGTCCT | |
| | TCTCCTGTCACTGGTTATCACCCTTTACTGCAAACGGGGCA | |
| | GAAAGAAACTCCTGTATATATTCAAACAACCATTTATGAG | |
| | ACCAGTACAAACTACTCAAGAGGAAGATGGCTGTAGCTGC | |
| | CGATTTCCAGAAGAAGAAGAAGGAGGATGTGAACTGAGA | |
| | GTGAAGTTCAGCAGGAGCGCAGACGCCCCCGCGTACCAGC | |
| | AGGGCCAGAACCAGCTCTATAACGAGCTCAATCTAGGACG | |
| | AAGAGAGGAGTACGATGTTTTGGACAAGAGACGTGGCCGG | |
| | GACCCTGAGATGGGGGGAAAGCCGAGAAGGAAGAACCCT | |
| | CAGGAAGGCCTGTACAATGAACTGCAGAAAGATAAGATGG | |
| | CGGAGGCCTACAGTGAGATTGGGATGAAAGGCGAGCGCCG | |
| | GAGGGGCAAGGGGCACGATGGCCTTTACCAGGGTCTCAGT | |
| | ACAGCCACCAAGGACACCTACGACGCCCTTCACATGCAGG | |
| | CCCTGCCCCCTCGCTAA | |

BCMA CAR3

| | | |
|---|---|---|
| BCMA CAR3 | MALPVTALLLPLALLLHAARPDIVLTQSPPSLAMSLGKRATIS | 15502 |
| scFv (amino | CRASESVTILGSHLIHWYQQKPGQPPTLLIQLASNVQTGVPAR | |
| acid) | FSGSGSRTDFTLTIDPVEEDDVAVYYCLQSRTIPRTFGGGTKLE | |
| | IKGSTSGSGKPGSGEGSTKGQIQLVQSGPELKKPGETVKISCK | |
| | ASGYTFTDYSINWVKRAPGKGLKWMGWINTETREPAYAYDF | |
| | RGRFAFSLETSASTAYLQINNLKYEDTATYFCALDYSYAMDY | |
| | WGQGTSVTVSS | |

BCMA CAR4

| | | |
|---|---|---|
| BCMA CAR4 | MALPVTALLLPLALLLHAARPDIVLTQSPPSLAMSLGKRATIS | 15503 |
| Full CAR | CRASESVTILGSHLIHWYQQKPGQPPTLLIQLASNVQTGVPAR | |
| (amino acid) | FSGSGSRTDFTLTIDPVEEDDVAVYYCLQSRTIPRTFGGGTKLE | |
| | IKGSTSGSGKPGSGEGSTKGQIQLVQSGPELKKPGETVKISCK | |
| | ASGYTFTDYSINWVKRAPGKGLKWMGWINTETREPAYAYDF | |
| | RGRFAFSLETSASTAYLQINNLKYEDTATYFCALDYSYAMDY | |
| | WGQGTSVTVSSFVPVFLPAKPTTTPAPRPPTPAPTIASQPLSLR | |
| | PEACRPAAGGAVHTRGLDFACDIYIWAPLAGTCGVLLLSLVIT | |
| | LYCNHRNKRGRKKLLYIFKQPFMRPVQTTQEEDGCSCRFPEE | |
| | EEGGCELRVKFSRSADAPAYQQGQNQLYNELNLGRREEYDV | |
| | LDKRRGRDPEMGGKPRRKNPQEGLYNELQKDKMAEAYSEIG | |
| | MKGERRRGKGHDGLYQGLSTATKDTYDALHMQALPPR | |
| BCMA CAR4 | ATGGCTCTGCCTGTGACAGCTCTGCTGCTGCCTCTGGCCCT | 15504 |
| Full CAR | GCTGCTGCATGCCGCCAGACCTGATATCGTGCTGACCCAGA | |
| (nucleic acid) | GCCCTCCCAGCCTGGCCATGTCTCTGGGCAAGAGAGCCAC | |
| | CATCAGCTGCAGAGCCAGCGAGAGCGTGACCATCCTGGGC | |
| | AGCCACCTGATCCACTGGTATCAGCAGAAGCCCGGCCAGC | |
| | CCCCTACCCTGCTGATTCAGCTGGCCTCCAATGTGCAGACC | |
| | GGCGTGCCAGCCAGATTTTCCGGCAGCGGCAGCAGAACCG | |
| | ACTTCACCCTGACCATCGACCCCGTGGAAGAGGACGACGT | |
| | GGCCGTGTACTACTGCCTGCAGAGCAGAACCATCCCCCGG | |
| | ACCTTTGGCGGAGGCACCAAGCTGGAAATCAAGGGCAGCA | |
| | CCAGCGGCTCCGGCAAGCCTGGATCTGGCGAGGGATCTAC | |
| | CAAGGGACAGATCCAGCTGGTGCAGAGCGGCCCTGAGCTG | |
| | AAGAAACCCGGCGAGACAGTGAAGATCTCCTGCAAGGCCA | |
| | GCGGCTACACCTTCACCGACTACAGCATCAACTGGGTCAA | |
| | GAGGGCCCCTGGCAAGGGCCTGAAGTGGATGGGCTGGATC | |
| | AACACCGAAACCAGAGAGCCCGCCTACGCCTACGACTTCA | |
| | GAGGCAGATTCGCCTTCAGCCTGGAAACCAGCGCCAGCAC | |
| | CGCCTACCTGCAGATCAACAACCTGAAGTACGAGGACACC | |
| | GCCACCTACTTTTGCGCCCTGGACTACAGCTACGCCATGGA | |
| | CTATTGGGGCCAGGGCACCAGCGTGACCGTGTCCTCTTTCG | |
| | TGCCTGTGTTTCTGCCTGCCAAGCCCACCACAACCCCTGCC | |
| | CCTAGACCTCCTACACCCGCCCCTACAATCGCCAGCCAGCC | |
| | TCTGTCTCTGAGGCCCGAGGCTTGTAGACCTGCTGCTGGCG | |
| | GAGCCGTGCACACCAGAGGACTGGATTTCGCCTGCGACAT | |
| | CTACATCTGGGCCCCTCTGGCCGGCACATGTGGCGTGCTGC | |
| | TGCTGAGCCTCGTGATCACCCTGTACTGCAACCACCGGAAC | |
| | AAGCGGGGCAGAAAGAAGCTGCTGTACATCTTCAAGCAGC | |
| | CCTTCATGCGGCCCGTGCAGACCACCCAGGAAGAGGACGG | |
| | CTGCTCCTGCAGATTCCCCGAGGAAGAAGAAGGCGGCTGC | |
| | GAGCTGAGAGTGAAGTTCAGCAGAAGCGCCGACGCCCCTG | |
| | CCTATCAGCAGGGCCAGAACCAGCTGTACAACGAGCTGAA | |
| | CCTGGGCAGACGGGAAGAGTACGACGTGCTGGACAAGCGG | |
| | AGAGGCAGGGACCCTGAGATGGGCGGCAAGCCCAGAAGA | |
| | AAGAACCCCCAGGAAGGCCTGTATAACGAACTGCAGAAAG | |
| | ACAAGATGGCCGAGGCCTACAGCGAGATCGGAATGAAGG | |
| | GCGAGCGGAGAAGAGGCAAGGGCCACGATGGCCTGTACC | |
| | AGGGCCTGAGCACCGCCACCAAGGACACCTATGACGCCCT | |
| | GCACATGCAGGCCCTGCCCCCCAGATGA | |

BCMA CAR5

| | | |
|---|---|---|
| BCMA CAR5 | MALPVTALLLPLALLLHAARPEVQLLESGGGLVQPGGSLRLS | 15505 |
| Full CAR | CAASGFTFSSYAMSWVRQAPGKGLEWVSSISGSGDYIYYADS | |
| (amino acid) | VKGRFTISRDISKNTLYLQMNSLRAEDTAVYYCAKEGTGANS | |
| | SLADYRGQGTLVTVSSFVPVFLPAKPTTTPAPRPPTPAPTIASQ | |
| | PLSLRPEACRPAAGGAVHTRGLDFACDIYIWAPLAGTCGVLL | |
| | LSLVITLYCNHRNRSKRSRLLHSDYMNMTPRRPGPTRKHYQP | |
| | YAPPRDFAAYRSRVKFSRSADAPAYQQGQNQLYNELNLGRR | |
| | EEYDVLDKRRGRDPEMGGKPRRKNPQEGLYNELQKDKMAE | |
| | AYSEIGMKGERRRGKGHDGLYQGLSTATKDTYDALHMQALP | |
| | PR | |
| BCMA CAR5 | ATGGCCCTGCCTGTTACAGCTCTGCTGCTGCCTCTGGCTCT | 15506 |
| Full CAR | GCTTCTGCATGCCGCCAGACCTGAGGTGCAGCTGTTGGAGT | |
| (nucleic acid) | CTGGGGGAGGCTTGGTACAGCCTGGGGGGTCCCTGAGACT | |
| | CTCCTGTGCAGCCTCTGGATTCACCTTTAGCAGCTATGCCA | |
| | TGAGCTGGGTCCGCCAGGCTCCAGGGAAGGGGCTGGAGTG | |
| | GGTCTCATCTATTAGTGGTAGTGGTGATTACATATACTACG | |
| | CAGACTCCGTGAAGGGCCGGTTCACCATCTCCAGAGACAT | |
| | ATCCAAGAACACGCTGTATCTGCAAATGAACAGCCTGAGA | |
| | GCCGAGGACACGGCCGTATATTACTGTGCGAAAGAAGGTA | |
| | CGGGTGCCAACAGCAGCTTGGCAGACTACAGAGGCCAGGG | |
| | CACCCTGGTCACCGTCTCCTCATTCGTGCCCGTGTTCCTGCC | |
| | TGCCAAGCCTACAACAACCCCTGCTCCTAGACCTCCTACAC | |
| | CAGCTCCTACAATCGCCAGCCAGCCTCTGTCTCTGAGGCCT | |
| | GAAGCTTGTAGACCTGCTGCTGGCGGAGCCGTGCATACCA | |
| | GAGGACTGGATTTCGCCTGCGACATCTACATCTGGGCCCCT | |
| | CTGGCTGGAACATGTGGCGTTTTGCTGCTGAGCCTCGTGAT | |
| | CACCCTGTACTGCAACCACCGGAACAGAAGCAAGCGGAGC | |
| | CGGCTGCTGCACAGCGACTACATGAACATGACCCCCAGAC | |
| | GGCCTGGCCCCACCAGAAAGCACTACCAGCCTTACGCCCC | |
| | TCCCAGAGACTTCGCCGCCTACCGGTCCAGAGTGAAGTTCA | |
| | GCAGAAGCGCCGACGCCCCTGCCTATCAGCAGGGCCAGAA | |
| | CCAGCTGTACAACGAGCTGAACCTGGGCAGACGGGAAGAG | |
| | TACGATGTGCTGGACAAAAGACGTGGCCGGGACCCTGAGA | |
| | TGGGGGGAAAGCCGAGAAGGAAGAACCCTCAGGAAGGCC | |
| | TGTACAATGAACTGCAGAAAGATAAGATGGCGGAGGCCTA | |
| | CAGTGAGATTGGGATGAAAGGCGAGCGCCGGAGGGGCAA | |
| | GGGGCACGATGGCCTTTACCAGGGTCTCAGTACAGCCACC | |
| | AAGGACACCTACGACGCCCTTCACATGCAGGCCCTGCCCC | |
| | CTCGCTAA | |

BCMA CAR6

| | | |
|---|---|---|
| BCMA CAR6 | MALPVTALLLPLALLLHAARPEVQLLESGGGLVQPGGSLRLS | 15507 |
| Full CAR | CAASGFTFSSYAMSWVRQAPGKGLEWVSSISGSGDYIYYADS | |
| (amino acid) | VKGRFTISRDISKNTLYLQMNSLRAEDTAVYYCAKEGTGANS | |
| | SLADYRGQGTLVTVSSFVPVFLPAKPTTTPAPRPPTPAPTIASQ | |
| | PLSLRPEACRPAAGGAVHTRGLDFACDIYIWAPLAGTCGVLL | |
| | LSLVITLYCNHRNKRGRKKLLYIFKQPFMRPVQTTQEEDGCSC | |
| | RFPEEEEGGCELRVKFSRSADAPAYQQGQNQLYNELNLGRRE | |
| | EYDVLDKRRGRDPEMGGKPRRKNPQEGLYNELQKDKMAEA | |
| | YSEIGMKGERRRGKGHDGLYQGLSTATKDTYDALHMQALPP | |
| | R | |
| BCMA CAR6 | ATGGCCCTGCCTGTTACAGCTCTGCTGCTGCCTCTGGCTCT | 15508 |
| Full CAR | GCTTCTGCATGCCGCCAGACCTGAGGTGCAGCTGTTGGAGT | |
| (nucleic acid) | CTGGGGGAGGCTTGGTACAGCCTGGGGGGTCCCTGAGACT | |
| | CTCCTGTGCAGCCTCTGGATTCACCTTTAGCAGCTATGCCA | |
| | TGAGCTGGGTCCGCCAGGCTCCAGGGAAGGGGCTGGAGTG | |
| | GGTCTCATCTATTAGTGGTAGTGGTGATTACATATACTACG | |
| | CAGACTCCGTGAAGGGCCGGTTCACCATCTCCAGAGACAT | |
| | ATCCAAGAACACGCTGTATCTGCAAATGAACAGCCTGAGA | |
| | GCCGAGGACACGGCCGTATATTACTGTGCGAAAGAAGGTA | |
| | CGGGTGCCAACAGCAGCTTGGCAGACTACAGAGGCCAGGG | |
| | CACCCTGGTCACCGTCTCCTCATTCGTGCCCGTGTTCCTGCC | |
| | TGCCAAGCCTACAACAACCCCTGCTCCTAGACCTCCTACAC | |
| | CAGCTCCTACAATCGCCAGCCAGCCTCTGTCTCTGAGGCCT | |
| | GAAGCTTGTAGACCTGCTGCTGGCGGAGCCGTGCATACCA | |
| | GAGGACTGGATTTCGCCTGCGACATCTACATCTGGGCCCCT | |
| | CTGGCTGGAACATGTGGCGTTTTGCTGCTGAGCCTCGTGAT | |
| | CACCCTGTACTGCAACCACCGGAACAAGCGGGGCAGAAAG | |
| | AAGCTGCTGTACATCTTCAAGCAGCCCTTCATGCGGCCCGT | |
| | GCAGACCACCCAGGAAGAGGACGGCTGCTCCTGCAGATTC | |
| | CCCGAGGAAGAAGAAGGCGGCTGCGAGCTGAGAGTGAAG | |
| | TTCAGCAGAAGCGCCGACGCCCCTGCCTATCAGCAGGGCC | |
| | AGAACCAGCTGTACAACGAGCTGAACCTGGGCAGACGGGA | |
| | AGAGTACGACGTGCTGGACAAGCGGAGAGGCAGGGACCCT | |
| | GAGATGGGCGGCAAGCCCAGAAGAAAGAACCCCCAGGAA | |
| | GGCCTGTATAACGAACTGCAGAAAGACAAGATGGCCGAGG | |
| | CCTACAGCGAGATCGGAATGAAGGGCGAGCGGAGAAGAG | |
| | GCAAGGGCCACGATGGCCTGTACCAGGGCCTGAGCACCGC | |
| | CACCAAGGACACCTATGACGCCCTGCACATGCAGGCCCTG | |
| | CCCCCCAGATGA | |

BCMA CAR7

| | | |
|---|---|---|
| BCMA CAR7 | QVQLVQSGAEVKKPGASVKVSCKASGYSFPDYYINWVRQAP | 15509 |
| VH (amino | GQGLEWMGWIYFASGNSEYNQKFTGRVTMTRDTSSSTAYME | |
| acid) | LSSLRSEDTAVYFCASLYDYDWYFDVWGQGTMVTVSS | |
| BCMA CAR 7 | DIVMTQTPLSLSVTPGEPASISCKSSQSLVHSNGNTYLHWYLQ | 15510 |
| VL (amino | KPGQSPQLLIYKVSNRFSGVPDRFSGSGSGADFTLKISRVEAE | |
| acid) | DVGVYYCAETSHVPWTFGQGTKLEIK | |

BCMA CAR-8

| | | |
|---|---|---|
| BCMA CAR-8 | MALPVTALLLPLALLLHAARP | 15542 |
| CD8α signal | | |
| peptide (amino | | |
| acid) | | |
| BCMA CAR-8 | EVQLLESGGGLVQPGGSLRLSCAASGFTFSSYAMNWVRQAP | 15551 |
| VH (amino | GKGLEWVSAISDSGGSTYYADSVKGRFTISRDNSKNTLYLQ | |
| acid) | MNSLRAEDTAVYYCARYWPMDIWGQGTLVTVSS | |
| BCMA CAR-8 | GGGGSGGGGSGGGGS | 109 |
| GS linker | | |
| (amino acid) | | |
| BCMA CAR-8 | EIVLTQSPGTLSLSPGERATLSCRASQSVSSSYLAWYQQKPGQ | 15552 |
| VL (amino | APRLLMYDASIRATGIPDRFSGSGSGTDFTLTISRLEPEDFAVY | |
| acid) | YCQQYGSWPLTFGQGTKVEIK | |
| BCMA CAR-8 | GSGGGGSCPYSNPSLCSGGGGSCPYSNPSLCSGGGGS | 15553 |
| CD20 epitope | | |
| (amino acid) | | |
| BCMA CAR-8 | TTTPAPRPPTPAPTIASQPLSLRPEACRPAAGGAVHTRGLDFAC | 1101 |
| CD8α hinge | D | |
| (amino acid) | | |
| BCMA CAR-8 | IYIWAPLAGTCGVLLLSLVITYC | 15554 |
| CD8α | | |
| transmembrane | | |
| domain (amino | | |
| acid) | | |
| BCMA CAR-8 | KRGRKKLLYIFKQPFMRPVQTTQEEDGCCRFPEEEEGGCEL | 15480 |
| 4-1BB | | |
| intracellular | | |
| signaling | | |
| domain (amino | | |
| acid) | | |
| BCMA CAR-8 | RVKFSRSADAPAYQQGQNQLYNELNLGRREEYDVLDKRRGR | 1104 |
| CD3z | DPEMGGKPRRKNPQEGLYNELQKDKMAEAYSEIGMKGERRR | |
| instracellular | GKGHDGLYQGLSTATKDTYDALHMQALPPR | |
| signaling | | |
| domain (amino | | |
| acid) | | |
| BCMA CAR-8 | MALPVTALLLPLALLLHAARPEVQLLESGGGLVQPGGSLRLS | 15555 |
| Full CAR | CAASGFTFSSYAMNWVRQAPGKGLEWVSAISDSGGSTYYAD | |
| (amino acid) | SVKGRFTISRDNSKNTLYLQMNSLRAEDTAVYYCARYWPMD | |
| | IWGQGTLVTVSSGGGGSGGGGSGGGGSEIVLTQSPGTLSLSPG | |
| | ERATLSCRASQSVSSSYLAWYQQKPGQAPRLLMYDASIRATG | |
| | IPDRFSGSGSGTDFTLTISRLEPEDFAVYYCQQYGSWPLTFGQ | |
| | GTKVEIKTTTPAPRPPTPAPTIASQPLSLRPEACRPAAGGAVHT | |
| | RGLDFACDIYIWAPLAGTCGVLLLSLVITLYCKRGRKKLLYIF | |
| | KQPFMRPVQTTQEEDGCSCRFPEEEEGGCELRVKFSRSADAP | |
| | AYQQGQNQLYNELNLGRREEYDVLDKRRGRDPEMGGKPRR | |
| | KNPQEGLYNELQKDKMAEAYSEIGMKGERRRGKGHDGLYQ | |
| | GLSTATKDTYDALHMQALPPR | |
| BCMA CAR-8 | atggcactgcctgtgaccgccctgctgctgccactggccctgctgctgcacgccgcccggccag | 15556 |
| Full CAR | aggtgcagctgctggagagcggaggaggactggtgcagccaggaggaagcctgagactgtcc | |
| (nucleic acid) | tgcgccgcctctggcttcaccttcagctcctacgccatgaactgggtgaggcaggcaccaggcaa | |
| | gggactggagtgggtgtctgccatcctgtctagcggcggcagcacctactatgccgattccgtga | |
| | agggccgcttcacaatcagccgggacaactccaagaataccctgtacctgcagatgaacagcct | |
| | gagagccgaggatacagccgtgtactattgcgccaggtattggcccatggacatctggggccag | |
| | ggcacactggtgaccgtgtcctctggaggaggaggatccggcggaggaggctctggcggcgg | |
| | cggcagcgagatcgtgctgacacagagcccaggcaccctgagcctgtcccctggagagagag | |
| | ccaccctgtcttgtaggggcggccagagcgtgagctcctcttacctggcctggtatcagcagaag | |
| | ccaggccaggcccccagactgctgatgtacgacgcctccatcagggcaacaggcatccccgat | |
| | cggttctctggaagcggatccggaaccgactttacactgaccatctccaggctggagcctgagga | |
| | tttcgccgtgtactattgccagcagtaccagtcttggccactgacatttggccagggcaccaaggt | |
| | ggagatcaagggatccggaggaggaggatcttgcccttattccaacccatctctgtgcaccacaac | |
| | ccctgcaccaaggccacctacaccagcacctaccatcgcctctcagccactgagcctgagaccc | |
| | gaggcctgtaggcctgcagcaggaggagcagtgcacacacggggactggactttgcctgcgat | |
| | atctacatctgggcacctctggcaggaacatgtggcgtgctgctgctgagcctggtcatcaccctg | |
| | tactgcaagagaggcaggaagaagctgctgtatatcttcaagcagccctttatgcgccctgtgcag | |
| | acaacccaggaggaggatggctgctcctgtaggttcccagaagaggaggagggaggatgtgag | |
| | ctgcgcgtgaagttttctcggagcgccgacgcacctgcataccagcagggacagaatcagctgta | |
| | taacgagctgaatctgggccggagagaggagtatgacgtgctggataagaggaggggaaggg | |
| | acccagagatgggaggcaagccacggagaaagaacccccaggagggcctgtacaatgagctg | |
| | cagaaggataagatggccgaggcctatagcgagatcggcatgaagggagagaggcgccggg | |
| | gcaagggacacgacggactgtaccagggactgtccacagccaccaaggacacctatgatgccc | |
| | tgcacatgcaggccctgccaccaaggtga | |

BCMA CAR-9

| | | |
|---|---|---|
| BCMA CAR-9 | EVQLLESGGGLVQPGGSLRLSCAASGFTFSSYPMSWVRQAPG | 15557 |
| VH (amino | KGLEWVSAIGGSGGSLPYADSVKGRFTISRDNSKNTLYLQM | |
| acid) | NSLRAEDTAVYYCARYWPMDIWGQGTLVTVSS | |
| BCMA CAR-9 | EIVLTQSPGTLSLSPGERATLSCRASQSVSSSYLAWYQQKPGQ | 15558 |
| VL (amino | APRLLMYDASIRATGIPDRFSGSGSGTDFTLTISRLEPEDFAVY | |
| acid) | YCQQYQSWPLTFGQGTKVEIK | |
| BCMA CAR-10 | | |
| BCMA CAR- | MGSWVEFWDRLGAIRERLEALGGSEAELAAFEKEIAAFESEL | 15559 |
| 10D domain | QAYKGKGNPEVEKLRYVAAVIRHRLQAYRHN | |
| (amino acid) | | |
| BCMA CAR-11 | | |
| BCMA CAR- | MGSWSEFWVRLGAIRERLDALGGSEAELAAFEKEIAAFESEL | 15560 |
| 11D domain | QAYKGKGNPEVEKLRYTAATIRRFLQAYRHN | |
| (amino acid) | | |
| BCMA CAR-12 | | |
| BCMA CAR- | MGSWSEFWARLGAIRERLDALGGSEABLAAFEKEIAAFESEL | 15561 |
| 12D domain | QAYKGKGNPEVEKLRYTAGTIKRFLQAYRHN | |
| (amino acid) | | |
| CD19 CAR1 | | |
| CD19 CAR1 | DIQMTQTTSSLSASLGDRVTISCRASQDISKYLNWYQQKPDGT | 15511 |
| VL (amino | VKLLIYHTSRLHSGVPSRFSGSGSGTDYSLTISNLEQEDIATYF | |
| acid) | CQQGNTLPYTFGGGTKLEIT | |
| CD19 CAR1 | GACATCCAGATGACACAGACTACATCCTCCCTGTCTGCCTC | 15512 |
| VL (nucleic | TCTGGGAGACAGAGTCACCATCAGTTGCAGGGCAAGTCAG | |
| acid) | GACATTAGTAAATATTTAAATTGGTATCAGCAGAAACCAG | |
| | ATGGAACTGTTAAACTCCTGATCTACCATACATCAAGATTA | |
| | CACTCAGGAGTCCCATCAAGGTTCAGTGGCAGTGGGTCTG | |
| | GAACAGATTATTCTCTCACCATTAGCAACCTGGAGCAAGA | |
| | AGATATTGCCACTTACTTTTGCCAACAGGGTAATACGCTTC | |
| | CGTACACGTTCGGAGGGGGGACTAAGTTGGAAATAACA | |
| CD19 CAR1 | EVKLQESGPGLVAPSQSLSVTCTVSGVSLPDYGVSWIRQPPRK | 15513 |
| VH (amino | GLEWLGVIWGSETTYYNSALKSRLTIIKDNSKSQVFLKMNSL | |
| acid) | QTDDTAIYYCAKHYYYGGSYAMDYWGQGTSVTVSS | |
| CD19 CAR1 | GAGGTGAAACTGCAGGAGTCAGGACCTGGCCTGGTGGCGC | 15514 |
| VH (nucleic | CCTCACAGAGCCTGTCCGTCACATGCACTGTCTCAGGGGTC | |
| acid) | TCATTACCCGACTATGGTGTAAGCTGGATTCGCCAGCCTCC | |
| | ACGAAAGGGTCTGGAGTGGCTGGGAGTAATATGGGGTAGT | |
| | GAAACCACATACTATAATTCAGCTCTCAAATCCAGACTGAC | |
| | CATCATCAAGGACAACTCCAAGAGCCAAGTTTTCTTAAAA | |
| | ATGAACAGTCTGCAAACTGATGACACAGCCATTTACTACTG | |
| | TGCCAAACATTATTACTACGGTGGTAGCTATGCTATGGACT | |
| | ACTGGGGTCAAGGAACCTCAGTCACCGTCTCCTCA | |
| CD19 CAR1 | DIQMTQTTSSLSASLGDRVTISCRASQDISKYLNWYQQKPDGT | 15521 |
| scFv (amino | VKLLIYHTSRLHSGVPSRFSGSGSGTDYSLTISNLEQEDIATYF | |
| acid) | CQQGNTLPYTFGGGTKLEITGSTSGSGKPGSGEGSTKGEVKLQ | |
| | ESGPGLVAPSQSLSVTCTVSGVSLPDYGVSWIRQPPRKGLEWL | |
| | GVIWGSETTYYNSALKSRLTIIKDNSKSQVFLKMNSLQTDDTA | |
| | IYYCAKHYYYGGSYAMDYWGQGTSVTVSS | |
| CD19 CAR1 | DIQMTQTTSSLSASLGDRVTISCRASQDISKYLNWYQQKPDGT | 15515 |
| Full CAR | VKLLIYHTSRLHSGVPSRFSGSGSGTDYSLTISNLEQEDIATYF | |
| (amino acid) | CQQGNTLPYTFGGGTKLEITGSTSGSGKPGSGEGSTKGEVKLQ | |
| | ESGPGLVAPSQSLSVTCTVSGVSLPDYGVSWIRQPPRKGLEWL | |
| | GVIWGSETTYYNSALKSRLTIIKDNSKSQVFLKMNSLQTDDTA | |
| | IYYCAKHYYYGGSYAMDYWGQGTSVTVSSTTTPAPRPPTPAP | |
| | TIASQPLSLRPEACRPAAGGAVHTRGLDFACDIYIWAPLAGTC | |
| | GVLLLSLVITLYCRSKRSRLLHSDYMNMTPRRPGPTRKHYQP | |
| | YAPPRDFAAYRSRVKFSRSADAPAYQQGQNQLYNELNLGRR | |
| | EEYDVLDKRRGRDPEMGGKPRRKNPQEGLYNELQKDKMAE | |
| | AYSEIGMKGERRRGKGHDGLYQGLSTATKDTYDALHMQALP | |
| | PRKRGRKKLLYIFKQPFMRPVQTTQEEDGCSCRFPEEEEGGCE | |
| | L | |
| CD19 CAR1 | GACATCCAGATGACACAGACTACATCCTCCCTGTCTGCCTC | 15516 |
| Full CAR | TCTGGGAGACAGAGTCACCATCAGTTGCAGGGCAAGTCAG | |
| (nucleic acid) | GACATTAGTAAATATTTAAATTGGTATCAGCAGAAACCAG | |
| | ATGGAACTGTTAAACTCCTGATCTACCATACATCAAGATTA | |
| | CACTCAGGAGTCCCATCAAGGTTCAGTGGCAGTGGGTCTG | |
| | GAACAGATTATTCTCTCACCATTAGCAACCTGGAGCAAGA | |
| | AGATATTGCCACTTACTTTTGCCAACAGGGTAATACGCTTC | |
| | CGTACACGTTCGGAGGGGGGACTAAGTTGGAAATAACAGG | |
| | CTCCACCTCTGGATCCGGCAAGCCCGGATCTGGCGAGGGA | |
| | TCCACCAAGGGCGAGGTGAAACTGCAGGAGTCAGGACCTG | |
| | GCCTGGTGGCGCCCTCACAGAGCCTGTCCGTCACATGCACT | |
| | GTCTCAGGGGTCTCATTACCCGACTATGGTGTAAGCTGGAT | |
| | TCGCCAGCCTCCACGAAAGGGTCTGGAGTGGCTGGGAGTA | |
| | ATATGGGGTAGTGAAACCACATACTATAATTCAGCTCTCAA | |
| | ATCCAGACTGACCATCATCAAGGACAACTCCAAGAGCCAA | |
| | GTTTTCTTAAAAATGAACAGTCTGCAAACTGATGACACAGC | |
| | CATTTACTACTGTGCCAAACATTATTACTACGGTGGTAGCT | |
| | ATGCTATGGACTACTGGGGTCAAGGAACCTCAGTCACCGT | |
| | CTCCTCAACCACTACCCCAGCACCGAGGCCACCCACCCCG | |
| | GCTCCTACCATCGCCTCCCAGCCTCTGTCCCTGCGTCCGGA | |
| | GGCATGTAGACCCGCAGCTGGTGGGGCCGTGCATACCCGG | |
| | GGTCTTGACTTCGCCTGCGATATCTACATTTGGGCCCCTCT | |
| | GGCTGGTACTTGCGGGGTCCTGCTGCTTTCACTCGTGATCA | |
| | CTCTTTACTGTAGGAGTAAGAGGAGCAGGCTCCTGCACAG | |
| | TGACTACATGAACATGACTCCCCGCCGCCCCGGGCCCACCC | |
| | GCAAGCATTACCAGCCCTATGCCCCACCACGCGACTTCGCA | |
| | GCCTATCGCTCCAGAGTGAAGTTCAGCAGGAGCGCAGACG | |
| | CCCCCGCGTACCAGCAGGGCCAGAACCAGCTCTATAACGA | |
| | GCTCAATCTAGGACGAAGAGAGGAGTACGATGTTTTGGAC | |
| | AAGAGACGTGGCCGGGACCCTGAGATGGGGGGAAAGCCG | |
| | AGAAGGAAGAACCCTCAGGAAGGCCTGTACAATGAACTGC | |
| | AGAAAGATAAGATGGCGGAGGCCTACAGTGAGATTGGGAT | |
| | GAAAGGCGAGCGCCGGAGGGGCAAGGGGCACGATGGCCT | |
| | TTACCAGGGTCTCAGTACAGCCACCAAGGACACCTACGAC | |
| | GCCCTTCACATGCAGGCCCTGCCCCCTCGCAAACGGGGCA | |
| | GAAAGAAACTCCTGTATATATTCAAACAACCATTTATGAG | |
| | ACCAGTACAAACTACTCAAGAGGAAGATGGCTGTAGCTGC | |
| | CGATTTCCAGAAGAAGAAGAAGGAGGATGTGAACTG | |

GPRC5D CAR1

| | | |
|---|---|---|
| GPRC5D | QMQLVQSGAEVKKPGASVKVSCKASGYTFNRYAITWVRQAP | 15522 |
| CAR1 VH | GQGLEWMGWISAYNGNSHYAQKLQGRVTMTTDTSTGTAYM | |
| (amino acid) | ELRRLRSDDTAVYYCARMAYDSWGQGTLVTVSS | |
| GPRC5D | cagatgcagctggtgcagtctggagctgaggtgaagaagcctggggcctcagtga | 15523 |
| CAR1 VH | aggtctcctgcaaggcttctggttacacctttaacagatatgctatcacctgggt | |
| (nucleic acid) | gcgacaggcccctggacaaggccttgagtggatgggatggatcagcgcttacaat | |
| | ggtaattcacactatgcacagaagctccagggcagagtcaccatgaccacagaca | |
| | catccacgggcacagcctatatggagctgaggaggctgagatctgacgacacggc | |
| | cgtgtattactgtgcgcgcatggcttacgattcttggggtcaaggtactctggtg | |
| | accgtctcctca | |
| GPRC5D | QSVLTQPASVSGSPGQSLTISCTGTSNDVGAYKYVSWYQQYP | 15524 |
| CAR1 VL | GKAPKLILYDVFKRPSGVSNRFSGSKSDNTASLTISGLQAEDE | |
| (amino acid) | ADYYCFSLTSSNTYVFGTGTKVTVLG | |
| GPRC5D | cagtctgtgttgacgcagcctgcctccgtgtctgggtctcctggacagtcgctcaccatctcctg | 15525 |
| CAR1 VL | cactggaaccagcaatgacgttggtgcttataagtatgtctcctggtatcaacagtacccaggca | |
| (nucleic acid) | aagcccccaaactcatactttatgatgtctttaagcggccctcaggggtctctaatcgcttctct | |
| | ggctccaagtctgacaacacggcctccctgaccatctctgggctccaggctgaggacgaggctga | |
| | ttattactgcttctcacttacaagcagtaacacttatgtcttcggaactgggaccaaggtcaccg | |
| | tcctaggt | |
| GPRC5D | QSVLTQPASVSGSPGQSLTISCTGTSNDVGAYKYVSWYQQYP | 15526 |
| CAR1 scFv | GKAPKLILYDVFKRPSGVSNRFSGSKSDNTASLTISGLQAEDE | |
| (amino acid) | ADYYCFSLTSSNTYVFGTGTKVTVLGSRGGGGSGGGGSGGG | |
| | GSLEMAQMQLVQSGAEVKKPGASVKVSCKASGYTFNRYAIT | |
| | WVRQAPGQGLEWMGWISAYNGNSHYAQKLQGRVTMTTDTS | |
| | TGTAYMELRRLRSDDTAVYYCARMAYDSWGQGTLVTVSS | |

GPRC5D CAR2

| | | |
|---|---|---|
| GPRC5D | EVQLVESGGGLVKPGGSLRLSCAASGFTFSDYYMSWIRQAPG | 15527 |
| CAR2 VH | KGLEWVSYISSSGSTIYYADSVKGRFTISRDNAKNSLYLQMNS | |
| (amino acid) | LRAEDTAVYYCARGYGKAYDQWGQGTLVTVSS | |
| GPRC5D | Gaggtgcagctggtggagtctgggggaggcttggtcaagcctggagggtccctgagactctcct | 15528 |
| CAR2 VH | gtgcagcctctggattcaccttcagtgactactacatgagctggatccgccaggctccagggaag | |
| (nucleic acid) | gggctggagtgggtttcatacattagtagtagtggtagtaccatatactacgcagactctgtgaa | |
| | gggccgattcaccatctccagggacaacgccaagaactcactgtatctgcaaatgaacagcctga | |
| | gagccgaggacacggccgtatattactgtgcgcgcggttacggtaaagcttacgatcagtggggt | |
| | caaggtactctggtgaccgtctcctca | |
| GPRC5D | QSVLTQPPSASGTPGQRVTISCSGSRSNVGGNYVFWYQQVPG | 15529 |
| CAR2 VL | ATPKLLIYRSNQRPSGVPDRFAGSKSGSSASLAISGLRSEDEAD | |
| (amino acid) | YYCATWDDSLSGFVFGTGTKVTVLG | |
| GPRC5D | cagtctgtgttgactcagccaccctcagcgtctgggacccccggacagagggtcaccatctcttg | 15530 |
| CAR2 VL | ttctggaagcaggtccaacgtaggaggtaattatgtattttggtaccagcaagtccccggagcga | |
| (nucleic acid) | cccccaaactcctcatctataggagtaatcagcggccctcgggggtccctgaccgattcgctggc | |
| | tccaagtctggctcctcagcctccctggccatcagtggactccggtccgaggatgaggctgatta | |
| | ttactgtgcaacatgggatgacagcctgagtggttttgtcttcggaactgggaccaaggtcaccg | |
| | tcctaggt | |
| GPRC5D | QSVLTQPPSASGTPGQRVTISCSGSRSNVGGNYVFWYQQVPG | 15531 |
| CAR2 scFv | ATPKLLIYRSNQRPSGVPDRFAGSKSGSSASLAISGLRSEDEAD | |
| (amino acid) | YYCATWDDSLSGFVFGTGTKVTVLGSRGGGGSGGGGSGGGG | |
| | SLEMAEVQLVESGGGLVKPGGSLRLSCAASGFTFSDYYMSWI | |
| | RQAPGKGLEWVSYISSSGSTIYYADSVKGRFTISRDNAKNSLY | |
| | LQMNSLRAEDTAVYYCARGYGKAYDQWGQGTLVTVSS | |

GPRC5D CAR3

| | | |
|---|---|---|
| GPRC5D | QVQLVESGGGLVHPGGSLRLSCAASGFTFRSHSMNWVRQAP | 15532 |
| CAR3 VH | GKGLEWVSSISSDSTYTYYADSVKGRFTISRDNAKNSLYLQM | |
| (amino acid) | NSLRAEDTAVYYCARSGGQWKYYDYWGQGTLVTVSS | |
| GPRC5D | Caggtgcagctggtggagtctgggggaggcctggtccaccctggggggtccctgagactctcct | 15533 |
| CAR3 VH | gtgcagcctctggattcaccttcagaagccatagcatgaactgggtccgccaggctccagggaag | |
| (nucleic acid) | gggctggagtgggtctcatccattagtagtgatagtacttacacatactacgcagactcagtgaa | |
| | gggccgattcaccatctccagagacaacgccaagaactcactgtatctgcaaatgaacagcctga | |
| | gagccgaggacacggccgtatattactgtgcgcgctctggtggtcagtggaaatactacgattac | |
| | tggggtcaaggtactctggtgaccgtctcctca | |
| GPRC5D | SSELTQDPAVSVALGQTVRITCQGDSLRSYYASWYQQKPGQA | 15534 |
| CAR3 VL | PVLVIYGKNNRPSGIPDRFSGSSSGNTASLTITGAQAEDEADY | |
| (amino acid) | YCNSRDSSGNPPVVFGGGTKLTVLG | |
| GPRC5D | Tcttctgagctgactcaggaccctgctgtgtctgtggccttgggacagacagtcaggatcacatg | 15535 |
| CAR3 VL | ccaaggagacagcctcagaagctattatgcaagctggtaccagcagaagccaggacaggcccct | |
| (nucleic acid) | gtacttgtcatctatggtaaaaacaaccggccctcagggatcccagaccgattctctggctccag | |
| | ctcaggaaacacagcttccttgaccatcactggggctcaggcggaagatgaggctgactattact | |
| | gtaactcccgggacagcagtggtaacccccctgtggtattcggcggagggaccaagctgaccgtc | |
| | ctaggt | |
| GPRC5D | SSELTQDPAVSVALGQTVRITCQGDSLRSYYASWYQQKPGQA | 15536 |
| CAR3 scFv | PVLVIYGKNNRPSGIPDRFSGSSSGNTASLTITGAQAEDEADY | |
| (amino acid) | YCNSRDSSGNPPVVFGGGTKLTVLGSRGGGGSGGGGSGGGG | |
| | SLEMAQVQLVESGGGLVHPGGSLRLSCAASGFTFRSHSMNW | |
| | VRQAPGKGLEWVSSISSDSTYTYYADSVKGRFTISRDNAKNS | |
| | LYLQMNSLRAEDTAVYYCARSGGQWKYYDYWGQGTLVTVS | |
| | S | |

GPRC5D CAR4

| | | |
|---|---|---|
| GPRC5D | EVQLVESGGGLIQPGGSLRLSCAASGFTFSNYAMNWVRQAPG | 15537 |
| CAR4 VH | KGLEWVSTINGRGSSTIYADSVKGRFTISRDNSKNTLYLQMNS | |
| (amino acid) | LRAEDTATYYCARYISRGLGDSWGQGTLVTV | |
| GPRC5D | Gaggtgcagctggtggagtccgggggaggcttgatacagcctggggggtccctgagactctcc | 15538 |
| CAR4 VH | tgtgcagcctctggattcacctttagcaactatgccatgaactgggtccgccaggctccagggaa | |
| (nucleic acid) | ggggctggagtgggtctcaactattaatggtcgtggtagtagtacaatctacgcagactccgtga | |
| | agggccggttcaccatctccagagacaattccaagaacacgctgtatctgcaaatgaacagcctg | |
| | agagccgaggacacagccacgtattactgtgcgcgctacatctctcgtggtctgggtgattcttg | |
| | gggtcaaggtactctcgtgaccgtctcctca | |
| GPRC5D | QSVVTQPPSMSAAPGQQVTISCSGGNSNIERNYVSWYLQLPG | 15539 |
| CAR4 VL | TAPKLVIFDNDRRPSGIPDRFSGSKSGTSATLGITGLQTGDEAD | |
| (amino acid) | YYCGTWDSSLRGWVFGGGTKLTVLG | |
| GPRC5D | Cagtctgtogtgacgcagccgccctcaatgtctgcggccccaggacagcaagtcaccatctcct | 15540 |
| CAR4 VL | gctctggaggcaactccaacattgagagaaattatgtatcctggtacctccagctccctggaaca | |
| (nucleic acid) | gcccccaaactcgtcatttttgacaatgataggcgaccctcagggattcctgaccgattctctgg | |
| | ctccaagtctggcacgtcagccaccctgggcatcaccggactccagactggggacgaggccgatt | |
| | attactgcggaacatgggatagcagcctgagaggttgggtgttcggcggagggaccaagctgacc | |
| | gtcctaggt | |
| GPRC5D | QSVVTQPPSMSAAPGQQVTISCSGGNSNIERNYVSWYLQLPG | 15541 |
| CAR4 scFv | TAPKLVIFDNDRRPSGIPDRFSGSKSGTSATLGITGLQTGDEAD | |
| (amino acid) | YYCGTWDSSLRGWVFGGGTKLTVLGSRGGGGSGGGGSGGG | |
| | GSLEMAEVQLVESGGGLIQPGGSLRLSCAASGFTFSNYAMNW | |
| | VRQAPGKGLEWVSTINGRGSSTIYADSVKGRFTISRDNSKNTL | |
| | YLQMNSLRAEDTATYYCARYISRGLGDSWGQGTLVTV | |
| CD20 CAR 1 | IVLTQSPAILSASPGEKVTMTCRASSSVNYMDWYQKKPGSSP | 15546 |
| scFv (amino | KPWIYATSNLASGVPARFSGSGSGTSYSLTISRVEAEDAATYY | |
| acid) | CQQWSFNPPTFGGGTKLEIKGSTSGGGSGGGSGGGGSSEVQL | |
| | QQSGAELVKPGASVKMSCKASGYTFTSYNMHWVKQTPGQG | |
| | LEWIGAIYPGNGDTSYNQKFKGKATLTADKSSSTAYMQLSSL | |
| | TSEDSADYYCARSNYYGSSYWFFDVWGAGTTVTVSS | |
| CD20 CAR 1 | ATTGTGCTGACCCAATCTCCAGCTATCCTGTCTGCATCTCC | 15547 |
| scFv (nucleic | AGGGGAGAAGGTCACAATGACTTGCAGGGCCAGCTCAAGT | |
| acid) | GTAAATTACATGGACTGGTACCAGAAGAAGCCAGGATCCT | |
| | CCCCCAAACCCTGGATTTATGCCACATCCAACCTGGCTTCT | |
| | GGAGTCCCTGCTCGCTTCAGTGGCAGTGGGTCTGGGACCTC | |
| | TTACTCTCTCACAATCAGCAGAGTGGAGGCTGAAGATGCT | |
| | GCCACTTATTACTGCCAGCAGTGGAGTTTTAATCCACCCAC | |
| | GTTCGGAGGGGGGACCAAGCTGGAAATAAAAGGCAGTACT | |
| | AGCGGTGGTGGCTCCGGGGGCGGTTCCGGTGGGGGCGGCA | |
| | GCAGCGAGGTGCAGCTGCAGCAGTCTGGGGCTGAGCTGGT | |
| | GAAGCCTGGGGCCTCAGTGAAGATGTCCTGCAAGGCTTCT | |
| | GGCTACACATTTACCAGTTACAATATGCACTGGGTAAAGC | |
| | AGACACCTGGACAGGGCCTGGAATGGATTGGAGCTATTTA | |
| | TCCAGGAAATGGTGATACTTCCTACAATCAGAAGTTCAAA | |
| | GGCAAGGCCACATTGACTGCAGACAAATCCTCCAGCACAG | |
| | CCTACATGCAGCTCAGCAGCCTGACATCTGAGGACTCTGCG | |
| | GACTATTACTGTGCAAGATCTAATTATTACGGTAGTAGCTA | |
| | CTGGTTCTTCGATGTCTGGGGCGCAGGGACCACGGTCACCG | |
| | TCTCCTCA | |
| CD20 CAR 1 | METDTLLLWVLLLWVPGSTGDIVLTQSPAILSASPGEKVTMT | 15548 |
| Full CAR | CRASSSVNYMDWYQKKPGSSPKPWIYATSNLASGVPARFSGS | |
| (amino acid) | GSGTSYSLTISRVEAEDAATYYCQQWSFNPPTFGGGTKLEIKG | |
| | STSGGGSGGGSGGGGSSEVQLQQSGAELVKPGASVKMSCKA | |
| | SGYTFTSYNMHWVKQTPGQGLEWIGAIYPGNGDTSYNQKFK | |
| | GKATLTADKSSSTAYMQLSSLTSEDSADYYCARSNYYGSSY | |
| | WFFDVWGAGTTVTVSSESKYGPPCPPCPMFWVLVVVGGVLA | |
| | CYSLLVTVAFIIFWVRSKRSRLLHSDYMNMTPRRPGPTRKHY | |
| | QPYAPPRDFAAYRSFSRSADAPAYQQGQNQLYNELNLGRREE | |
| | YDVLDKRRGRDPEMGGKPRRKNPQEGLYNELQKDKMAEAY | |
| | SEIGMKGERRRGKGHDGLYQGLSTATKDTYDALHMQALPPR | |
| CD20 CAR 1 | ATGGAGACAGACACACTCCTGCTATGGGTGCTGCTGCTCTG | 15549 |
| Full CAR | GGTTCCAGGTTCCACAGGTGACATTGTGCTGACCCAATCTC | |
| (nucleic acid) | CAGCTATCCTGTCTGCATCTCCAGGGGAGAAGGTCACAAT | |
| | GACTTGCAGGGCCAGCTCAAGTGTAAATTACATGGACTGG | |
| | TACCAGAAGAAGCCAGGATCCTCCCCCAAACCCTGGATTT | |
| | ATGCCACATCCAACCTGGCTTCTGGAGTCCCTGCTCGCTTC | |
| | AGTGGCAGTGGGTCTGGGACCTCTTACTCTCTCACAATCAG | |
| | CAGAGTGGAGGCTGAAGATGCTGCCACTTATTACTGCCAG | |
| | CAGTGGAGTTTTAATCCACCCACGTTCGGAGGGGGGACCA | |
| | AGCTGGAAATAAAAGGCAGTACTAGCGGTGGTGGCTCCGG | |
| | GGGCGGTTCCGGTGGGGGCGGCAGCAGCGAGGTGCAGCTG | |
| | CAGCAGTCTGGGGCTGAGCTGGTGAAGCCTGGGGCCTCAG | |
| | TGAAGATGTCCTGCAAGGCTTCTGGCTACACATTTACCAGT | |
| | TACAATATGCACTGGGTAAAGCAGACACCTGGACAGGGCC | |
| | TGGAATGGATTGGAGCTATTTATCCAGGAAATGGTGATACT | |
| | TCCTACAATCAGAAGTTCAAAGGCAAGGCCACATTGACTG | |
| | CAGACAAATCCTCCAGCACAGCCTACATGCAGCTCAGCAG | |
| | CCTGACATCTGAGGACTCTGCGGACTATTACTGTGCAAGAT | |
| | CTAATTATTACGGTAGTAGCTACTGGTTCTTCGATGTCTGG | |
| | GGCGCAGGGACCACGGTCACCGTCTCCTCAGAGAGCAAGT | |
| | ACGGACCGCCCTGCCCCCCTTGCCCTATGTTCTGGGTGCTG | |
| | GTGGTGGTCGGAGGCGTGCTGGCCTGCTACAGCCTGCTGGT | |
| | CACCGTGGCCTTCATCATCTTTTGGGTGaggagtaagaggagcaggct | |
| | cctgcacagtgactacatgaacatgactccccgccgccccgggcccacccgcaagcattaccag | |
| | ccctatgccccaccacgcgacttcgcagcctatcgctccTTCAGCAGAAGCGCCG | |
| | ACGCCCCTGCCTACCAGCAGGGCCAGAATCAGCTGTACAA | |
| | CGAGCTGAACCTGGGCAGAAGGGAAGAGTACGACGTCCTG | |
| | GATAAGCGGAGAGGCCGGGACCCTGAGATGGGCGGCAAG | |
| | CCTCGGCGGAAGAACCCCCAGGAAGGCCTGTATAACGAAC | |
| | TGCAGAAAGACAAGATGGCCGAGGCCTACAGCGAGATCGG | |
| | CATGAAGGGCGAGCGGAGGCGGGGCAAGGGCCACGACGG | |
| | CCTGTATCAGGGCCTGTCCACCGCCACCAAGGATACCTACG | |
| | ACGCCCTGCACATGCAGGCCCTGCCCCCAAGG | |

In some embodiments, the anti-BCMA CAR comprises a BCMA binding domain (e.g., from Table C9), a CD8 hinge domain (e.g., from Table C8), a CD8 transmembrane domain (e.g., from Table C6A), and a 4-1BB costimulatory domain (e.g., from Table C7A). In some embodiments, the anti-BCMA CAR additionally comprises a CD3z signaling domain (e.g., from Table C7A). In some embodiments, the anti-BCMA CAR comprises a BCMA binding domain (e.g., from Table C9), a CD8 hinge domain (e.g., from Table C8), a CD8 transmembrane domain (e.g., from Table C6A), a 4-1BB costimulatory domain (e.g., from Table C7A), and a CD3z signaling domain (e.g., from Table C7A). In some embodiments, the anti-BCMA CAR comprises a BCMA binding domain (e.g., from Table C9), a CD20 epitope, a CD8 hinge domain (e.g., from Table C8), a CD8 transmembrane domain (e.g., from Table C6A), a 4-1BB costimulatory domain (e.g., from Table C7A), and a CD3z signaling domain (e.g., from Table C7A). In some embodiments, the BCMA binding domain is murine. In some embodiments, the BCMA binding domain is humanized. In some embodiments, the BCMA binding domain is human. In some embodiments, the anti-BCMA CAR comprises a BCMA binding domain that comprises an scFv. In some embodiments, the anti-BCMA CAR comprises a BCMA binding domain that comprises two VHH domains (e.g., two linked camelid VHH antigen binding domains, e.g., VHH1 and VHH2 from Table C9). In some embodiments, the anti-BCMA CAR comprises a BCMA binding domain that comprises a D domain.

In some embodiments, the anti-BCMA CAR comprises a BCMA binding domain of Ciltacabtagene autoleucel (Carvykti), CT103A, CART-ddBCMA, NXC-201, idecabtagene vicleucel, ALLO-715, MCARH171, MCM998, P-BCMA-101, CTX120, or PBCAR269A. In some embodiments, the anti-BCMA CAR comprises Ciltacabtagene autoleucel (Carvykti), CT103A, CART-ddBCMA, NXC-201, idecabtagene vicleucel, ALLO-715, MCARH171, MCM998, P-BCMA-101, CTX120, or PBCAR269A.

In some embodiments, the anti-GPRC5D CAR comprises a VL and a VH domain from Table C9. In some embodiments, the anti-GPRC5D CAR comprises a VL domain, a VH domain, or an scFv of GPRC5D CARL, GPRC5D CAR2, GPRC5D CAR3, or GPRC5D CAR4 in Table C9. In some embodiments, the anti-GPRC5D CAR comprises a VL domain, a VH domain, or an scFv of GPRC5D CAR1, GPRC5D CAR2, GPRC5D CAR3, or GPRC5D CAR4 in Table C9, a CD28 transmembrane domain (e.g., from Table C6A), and a 4-1BB costimulatory domain (e.g., from Table C7A). In some embodiments, the anti-GPRC5D CAR additionally comprises a CD3z signaling domain (e.g., from Table C7A). In some embodiments, the GPRC5D binding domain is murine. In some embodiments, the GPRC5D binding domain is humanized. In some embodiments, the GPRC5D binding domain is human. In some embodiments, the anti-GPRC5D CAR comprises a GPRC5D binding domain that comprises an scFv. In some embodiments, the anti-GPRC5D CAR comprises a BCMA binding domain that comprises two VHH domains (e.g., two linked camelid VHH antigen binding domains, e.g., VHH1 and VHH2 from Table C9). In some embodiments, the anti-GPRC5D CAR comprises a GPRC5D binding domain that comprises a D domain.

In some embodiments, the anti-GPRC5D CAR comprises a GPRC5D binding domain of MCARH109, BMS-986393, or RD138. In some embodiments, the anti-GPRCSD CAR comprises MCARH109, BMS-986393, or RD138.

In some embodiments, the anti-CD19 CAR comprises a CD19 binding domain of FMC63. In some embodiments, the anti-CD19 CAR comprises a VL domain and a VH domain from Table C9. In some embodiments, the anti-CD19 CAR comprises a CD19 binding domain of FMC63, a CD28 hinge domain, a CD28 transmembrance domain, a CD28 costimlatory domain, and a CD3z signaling domain. In some embodiments, the anti-CD19 CAR comprises a CD19 binding domain of FMC63, a CD8a hinge domain, a CD8a transmembrance domain, a 4-1BB costimlatory domain, and a CD3z signaling domain. In some embodiments, the anti-CD19 CAR comprises a CD19 binding domain of FMC63, an IgG4 hinge domain, a CD28 transmembrance domain, a 4-1BB costimlatory domain, and a CD3z signaling domain. In some embodiments, the anti-CD19 CAR comprises FMC63. In some embodiments, the anti-CD19 CAR comprises Axicabtagene ciloleucel, Brexucabtagene autoleucel, Tisagenlecleucel, or Lisocabtagene maraleucel. In some embodiments, the CD19 binding domain is murine. In some embodiments, the CD19 binding domain is humanized. In some embodiments, the CD19 binding domain is human. In some embodiments, the anti-CD19 CAR comprises a CD19 binding domain that comprises an scFv.

In some embodiments, the anti-CD20 CAR comprises a VL domain and a VH domain from Table C9. In some embodiments, the anti-CD20 CAR comprises a CD20 binding domain from Table C9. In some embodiments, the anti-CD20 CAR comprises a CD20 CAR from Table C9. In some embodiments, the CD20 binding domain is murine. In some embodiments, the CD20 binding domain is humanized. In some embodiments, the CD20 binding domain is human. In some embodiments, the anti-CD20 CAR comprises a CD20 binding domain that comprises a scFv.

In some embodiments, the anti-GPRC5D CAR comprises a VL domain and a VH domain from Table C9. In some embodiments, the anti-GPRC5D CAR comprises a GPRC5D binding domain from Table C9. In some embodiments, the anti-GPRC5D CAR comprises a GPRC5D CAR from Table C9. In some embodiments, the GPRC5D binding domain is murine. In some embodiments, the GPRC5D binding domain is humanized. In some embodiments, the anti-GPRC5D CAR comprises a GPRC5D binding domain that comprises a scFv.

In some embodiments, the CAR comprises two antigen binding domains that target different antigens on the surface of a cell, e.g., a neoplastic cell, e.g., a blood cancer cell such as one associated with a leukemia, a lymphoma, or a multiple myeloma. In some embodiments, a CAR-T cell is engineered to comprise two CARs with antigen binding domains that target a different antigen on the surface of a cell, e.g., a neoplastic cell, e.g., a blood cancer cell such as one associated with a leukemia, a lymphoma, or a multiple myeloma. In some embodiments, the antigen binding domains of the CAR target CD20 and CD22. In some embodiments, the antigen binding domains of the CAR target CD19 and CD20. In some embodiments, the antigen binding domains of the CAR target CD19 and CD22. In some embodiments, the antigen binding domains of the CAR target GPRC5D and BCMA.

### CAR Compositions, Methods of Manufacture, and Uses

Additionally provided herein is a system for modifying DNA of a mammalian cell (e.g., a T cell, e.g., a cytotoxic, helper, or regulatory T cell, e.g., a primary T cell) to express a CAR, the system comprising:
(a) a gene modifying polypeptide or a nucleic acid (e.g., DNA or mRNA) encoding the gene modifying polypeptide, as disclosed herein, and
(b) a template RNA (or DNA encoding the template RNA) comprising (i) a sequence that binds the polypeptide and (ii) a heterologous object sequence encoding a chimeric antigen receptor (CAR), wherein the CAR comprises an antigen-binding domain, a transmembrane domain, a first intracellular signaling domain, and a second intracellular signaling domain, as disclosed herein.

In some embodiments, the system comprises:
(a) a gene modifying polypeptide or a nucleic acid (e.g., DNA or mRNA) encoding the gene modifying polypeptide, and
(b) a template RNA (or DNA encoding the template RNA) comprising (i) a sequence that binds the polypeptide and (ii) a heterologous object sequence encoding a chimeric antigen receptor (CAR), wherein one or more of:
(i) the CAR comprises an antigen binding domain that binds one or more antigens of a blood cancer (e.g., a leukemia or lymphoma), wherein optionally the antigen is a B cell antigen;
(ii) the CAR comprises an antigen binding domain that binds one or more antigens of a solid tumor;
(iii) the CAR comprise an antigen binding domain of any one of Tables C1-C5 or C9;
(iv) the CAR comprise a linker domain of Table L1 (e.g., a linker of SEQ ID NO 15520);
(v) the CAR comprises a transmembrane domain of Table C6 or C6A;
(vi) the CAR comprises a hinge domain (e.g., a hinge domain of Table C8);
(vii) the CAR comprises an intracellular signaling domain of Table C7 or C7A;
(viii) the CAR comprises a costimulatory domain of Table C7 or C7A;
(ix) the CAR comprises an antigen binding domain which comprises an scFv, a Fab, a diabody, a D domain binder, a centryin, or one or more single domain antibodies (e.g., VHH domains); or
(x) the CAR comprises an amino acid sequence of Table C9 or an amino acid sequence according to any one of SEQ ID NOs: 1100, 15490, 15492, 15498, 15500, 15502, 15503, 15505, 15507, 15509 and 15510, 15555, 15557 and 15558, 15559, 15560, 15561, 15515, 15526, 15531, 15536, 15541, or 15548;
(xi) wherein the CAR comprises a first intracellular signaling domain and a second intracellular signaling domain.

In some embodiments, provided herein are a population of cells comprising immune effector cells (e.g., T cells, e.g., primary T cells) or regulatory T cell (e.g., primary T reg cells) comprising a plurality of copies of a gene encoding a CAR (“a CAR gene”). In some embodiments, less than less than or equal to 70%, 65%, 60%, or 55% of copies of the CAR gene in the population are situated within a gene endogenous to a cell of the population. In some embodiments, less than 10%, 9%, 8%, 7%, 6%, or 5% of copies of the CAR gene in the population are situated within an exon of a gene endogenous to a cell of the population. In some embodiments, less than 70%, 65%, 60%, 55%, or 50% of copies of the CAR gene in the population are situated within an intron of a gene endogenous to a cell of the population. In some embodiments, less than 10%, 9%, 8%, or 7% of copies of the CAR gene in the population are situated upstream of a gene (e.g., within 2 kb of a transcriptional start site (TSS)) endogenous to a cell of the population. In some embodiments, at least 20%, 25%, 30%, 35%, or 40% of copies of the CAR gene in the population are situated within an intergenic region endogenous to a cell of the population.

In some embodiments, at least 50%, 55%, 60%, 65%, 70%, 75%, 80%, 85%, 90%, 95%, 96%, 97%, 98%, 99%, or 100% of cells in the plurality comprises a single copy of the CAR gene. In some embodiments, each cell in the plurality comprises a single copy of the CAR gene. In some embodiments, at least 0.1% of cells in the population comprise the CAR gene.

In some embodiments, the cell population comprises one or more of cancer cells, regulatory T cells, monocytes, and NK cells. In some embodiments, the immune effector cells and/or regulatory immune cells comprise T cells, e.g., primary T cells. In some embodiments, the immune effector cells or regulatory T cells comprise a leukapheresis sample or an apheresis sample. In some embodiments, the population of cells is substantially free of lentivirus proteins. In some embodiments, the population of cells is substantially free of lentivirus nucleic acids.

Additionally provided herein are methods of modifying a mammalian cell to express a CAR. In some embodiments, the method involves contacting the cell (e.g., an immune effector cell or a regulatory T cell) with a system disclosed herein. In some embodiments, the immune effector cell is a cell that expresses one or more Fc receptors and mediates one or more effector functions. In some embodiments, the immune effector cell may include, but may not be limited to, one or more of a monocyte, macrophage, neutrophil, dendritic cell, eosinophil, mast cell, platelet, large granular lymphocyte, Langerhans' cell, natural killer (NK) cell, T-lymphocyte (e.g., T-cell), a Gamma delta T cell, B-lymphocyte (e.g., B-cell) and may be from any organism including but not limited to humans, mice, rats, rabbits, and monkeys.

In some embodiments, the regulatory T cell (Treg) is a cell that suppresses an immune response, e.g., to mediate homeostasis and induce immune tolerance. In some embodiments, the Treg cell may include, but may not be limited to, a natural Treg or induced Treg and may be from any organism including but not limited to humans, mice, rats, rabbits, and monkeys.

In some embodiments, the mammalian cell is a T cell, e.g., a primary T cell. In some embodiments, provided herein is a method of modifying the genome of a mammalian T cell (e.g., a primary T cell), the method comprising contacting the cell with:
(a) a gene modifying polypeptide comprising an amino acid sequence of Table R1 or a sequence at least 70%, 75%, 80%, 85%, 90%, 95%, 96%, 97%, 98%, or 99% identical thereto, or a nucleic acid (e.g., DNA or mRNA) encoding the gene modifying polypeptide, and
(b) a template RNA (or DNA encoding the template RNA) comprising (i) a sequence that binds the polypeptide and (ii) a heterologous object sequence (e.g., encoding a CAR).

In some embodiments, provided herein is a method of modifying the genome of a mammalian T cell (e.g., a primary T cell), the method comprising contacting the cell with:
(a) a gene modifying polypeptide comprising an amino acid sequence of Table R1 or a sequence having no more than 10, 20, 30, 40, 50, 60, 70, 80, 90, or 100 amino acid differences thereto, or a nucleic acid (e.g., DNA or mRNA) encoding the gene modifying polypeptide, and
(b) a template RNA (or DNA encoding the template RNA) comprising (i) a sequence that binds the polypeptide and (ii) a heterologous object sequence (e.g., encoding a CAR).

In some embodiments, the method is performed ex vivo or in vitro.

Additionally provided herein is a reaction mixture comprising a gene modifying system disclosed herein (e.g., comprising a heterologous object sequence encoding a chimeric antigen receptor (CAR)) and a mammalian cell (e.g., a T cell, e.g., a primary T cell).

In some embodiments, the reaction mixture comprises a gene modifying polypeptide comprising an amino acid sequence of Table R1 or a sequence at least 70%, 75%, 80%, 85%, 90%, 95%, 96%, 97%, 98%, or 99% identical thereto, or a nucleic acid (e.g., DNA or mRNA) encoding the gene modifying polypeptide and a a mammalian T cell (e.g., a primary T cell). In some embodiments, the reaction mixture additionally includes a template RNA (or DNA encoding the template RNA) comprising (i) a sequence that binds the polypeptide and (ii) a heterologous object sequence.

In some embodiments, the reaction mixture comprises a gene modifying polypeptide comprising an amino acid sequence of Table R1 or a sequence no more than 10, 20, 30, 40, 50, 60, 70, 80, 90, or 100 amino acid differences thereto, or a nucleic acid (e.g., DNA or mRNA) encoding the gene modifying polypeptide and a mammalian T cell (e.g., a primary T cell). In some embodiments, the reaction mixture additionally includes a template RNA (or DNA encoding the template RNA) comprising (i) a sequence that binds the polypeptide and (ii) a heterologous object sequence.

In some embodiments, the systems, reaction mixtures, and cell populations modified using the methods disclosed herein can be used to treat a subject in need thereof. In some embodiments, the subject has a cancer, e.g., a hematological cancer or a solid tumor. In some embodiments, the subject has an infectious disease. In other embodiments, the subject has an autoimmune or an inflammatory disease.

### Compositions and Methods for Modifying Mammalian Cells

In some embodiments, provided herein are methods of modifying mammalian cells and reaction mixtures and systems for the same.

In some embodiments, provided herein is a method of modifying the genome of a mammalian T cell (e.g., a primary T cell), the method comprising contacting the cell with:
(a) a gene modifying polypeptide comprising an amino acid sequence of Table R1 or E14 or a sequence at least 70%, 75%, 80%, 85%, 90%, 95%, 96%, 97%, 98%, or 99% identical thereto, or a nucleic acid (e.g., DNA or mRNA) encoding the gene modifying polypeptide, and
(b) a template RNA (or DNA encoding the template RNA) comprising (i) a sequence that binds the polypeptide and (ii) a heterologous object sequence.

In some embodiments, provided herein is a method of modifying the genome of a mammalian T cell (e.g., a primary T cell), the method comprising contacting the cell with a system comprising:
(a) a gene modifying polypeptide comprising an amino acid sequence of Table R1 or E14 or a sequence having no more than 10, 20, 30, 40, 50, 60, 70, 80, 90, or 100 amino acid differences thereto, or a nucleic acid (e.g., DNA or mRNA) encoding the gene modifying polypeptide, and
(b) a template RNA (or DNA encoding the template RNA) comprising (i) a sequence that binds the polypeptide and (ii) a heterologous object sequence.

In some embodiments, the method is performed ex vivo or in vitro. In some embodiments, the gene modifying polypeptide and/or template RNA are formulated with an LNP.

In some embodiments, contacting the cell with the system results in insertion of the heterologous object sequence into at least 1%, 5%, 10%, 15%, 20%, 25%, or 30% of T cells.

Additionally provided herein is a reaction mixture comprising a gene modifying system disclosed herein (e.g., comprising a heterologous object sequence) and a mammalian cell (e.g., a T cell, e.g., a primary T cell).

In some embodiments, the reaction mixture comprises a gene modifying polypeptide comprising an amino acid sequence of Table R1 or E14 or a sequence at least 70%, 75%, 80%, 85%, 90%, 95%, 96%, 97%, 98%, or 99% identical thereto, or a nucleic acid (e.g., DNA or mRNA) encoding the gene modifying polypeptide and a mammalian T cell (e.g., a primary T cell). In some embodiments, the reaction mixture additionally includes a template RNA (or DNA encoding the template RNA) comprising (i) a sequence that binds the polypeptide and (ii) a heterologous object sequence.

In some embodiments, the reaction mixture comprises a gene modifying polypeptide comprising an amino acid sequence of Table R1 or E14 or a sequence no more than 10, 20, 30, 40, 50, 60, 70, 80, 90, or 100 amino acid differences thereto, or a nucleic acid (e.g., DNA or mRNA) encoding the gene modifying polypeptide and a mammalian T cell (e.g., a primary T cell). In some embodiments, the reaction mixture additionally includes a template RNA (or DNA encoding the template RNA) comprising (i) a sequence that binds the polypeptide and (ii) a heterologous object sequence.

Additionally provided herein is a population of T cells produced according to a method disclosed herein. In some embodiments, the population comprises a plurality of copies of the heterologous object sequence, wherein less than or equal to 70%, 65%, 60%, or 55% of copies of the heterologous object sequence in the population are situated within a gene endogenous to a cell of the population. In some embodiments, less than 10%, 9%, 8%, 7%, 6%, or 5% of copies of the heterologous object sequence in the population are situated within an exon of a gene endogenous to a cell of the population. In some embodiments, less than 70%, 65%, 60%, 55%, or 50% of copies of the heterologous object sequence in the population are situated within an intron of a gene endogenous to a cell of the population. In some embodiments, less than 10%, 9%, 8%, or 7% of copies of the heterologous object sequence in the population are situated upstream of a gene (e.g., within 2 kb of a transcriptional start site (TSS)) endogenous to a cell of the population. In some embodiments, at least 20%, 25%, 30%, 35%, or 40% of copies of the heterologous object sequence in the population are situated within an intergenic region endogenous to a cell of the population.

In some aspects, provided herein is a method of modifying the genome of a mammalian induced pluripotent stem cell (iPSC), the method comprising contacting the cell with:
(a) a gene modifying polypeptide, or a nucleic acid (e.g., DNA or mRNA) encoding the gene modifying polypeptide, and
(b) a template RNA (or DNA encoding the template RNA) comprising (i) a sequence that binds the polypeptide and (ii) a heterologous object sequence.

In some embodiments, the method results in insertion of the heterologous object sequence into at least 5%, 10%, or 15% of iPSCs.

In some aspects, provided herein is a method of modifying the genome of a mammalian iPSC, the method comprising contacting the cell with a system comprising:
(a) a gene modifying polypeptide comprising an amino acid sequence of Table R1 or E14 or a sequence having no more than 10, 20, 30, 40, 50, 60, 70, 80, 90, or 100 amino acid differences thereto, or a nucleic acid (e.g., DNA or mRNA) encoding the gene modifying polypeptide, and
(b) a template RNA (or DNA encoding the template RNA) comprising (i) a sequence that binds the polypeptide and (ii) a heterologous object sequence.

In some embodiments, the method is performed ex vivo or in vitro. In some embodiments, the gene modifying polypeptide and/or template RNA are formulated with an LNP.

In some embodiments, contacting the cell with the system results in insertion of the heterologous object sequence into at least 1%, 5%, 10%, 15%, 20%, 25%, or 30% of iPSCs.

Additionally provided herein is a reaction mixture comprising a gene modifying system disclosed herein (e.g., comprising a heterologous object sequence) and one or more iPSCs.

In some embodiments, the reaction mixture comprises a gene modifying polypeptide, or a nucleic acid (e.g., DNA or mRNA) encoding the gene modifying polypeptide and an iPSC. In some embodiments, the reaction mixture further comprises a template RNA (or DNA encoding the template RNA) comprising (i) a sequence that binds the polypeptide and (ii) a heterologous object sequence.

In some embodiments, the reaction mixture comprises a gene modifying polypeptide comprising an amino acid sequence of Table R1 or E14 or a sequence at least 70%, 75%, 80%, 85%, 90%, 95%, 96%, 97%, 98%, or 99% identical thereto, or a nucleic acid (e.g., DNA or mRNA) encoding the gene modifying polypeptide and an iPSC. In some embodiments, the reaction mixture additionally includes a template RNA (or DNA encoding the template RNA) comprising (i) a sequence that binds the polypeptide and (ii) a heterologous object sequence.

In some embodiments, the reaction mixture comprises a gene modifying polypeptide comprising an amino acid sequence of Table R1 or E14 or a sequence no more than 10, 20, 30, 40, 50, 60, 70, 80, 90, or 100 amino acid differences thereto, or a nucleic acid (e.g., DNA or mRNA) encoding the gene modifying polypeptide and an iPSC. In some embodiments, the reaction mixture additionally includes a template RNA (or DNA encoding the template RNA) comprising (i) a sequence that binds the polypeptide and (ii) a heterologous object sequence.

Additionally provided herein is a population of iPSCs produced according to a method disclosed herein. In some embodiments, the population comprises a plurality of copies of the heterologous object sequence, wherein less than or equal to 70%, 65%, 60%, or 55% of copies of the heterologous object sequence in the population are situated within a gene endogenous to a cell of the population. In some embodiments, less than 10%, 9%, 8%, 7%, 6%, or 5% of copies of the heterologous object sequence in the population are situated within an exon of a gene endogenous to a cell of the population. In some embodiments, less than 70%, 65%, 60%, 55%, or 50% of copies of the heterologous object sequence in the population are situated within an intron of a gene endogenous to a cell of the population. In some embodiments, less than 10%, 9%, 8%, or 7% of copies of the heterologous object sequence in the population are situated upstream of a gene (e.g., within 2 kb of a transcriptional start site (TSS)) endogenous to a cell of the population. In some embodiments, at least 20%, 24%, 30%, 35%, or 40% of copies of the heterologous object sequence in the population are situated within an intergenic region endogenous to a cell of the population.

In some aspects, provided herein is a method of modifying the genome of a mammalian respiratory epithelial cell (e.g., a bronchial epithelial cell, e.g., a human bronchial epithelial (hBE) cell), the method comprising contacting the cell with:
(a) a gene modifying polypeptide, or a nucleic acid (e.g., DNA or mRNA) encoding the gene modifying polypeptide, and
(b) a template RNA (or DNA encoding the template RNA) comprising (i) a sequence that binds the polypeptide and (ii) a heterologous object sequence.

In some embodiments, the method results in insertion of the heterologous object sequence into at least 5%, 10%, 15%, 20%, 30%, 40%, 50%, 60%, or 70% of respiratory epithelial cells (e.g., bronchial epithelial cells, e.g., human bronchial epithelial (hBE) cells).

In some aspects, provided herein is a method of modifying the genome of a mammalian respiratory epithelial cell (e.g., a bronchial epithelial cell, e.g., a human bronchial epithelial (hBE) cell), the method comprising contacting the cell with a system comprising:
(a) a gene modifying polypeptide comprising an amino acid sequence of Table R1 or E14 or a sequence having no more than 10, 20, 30, 40, 50, 60, 70, 80, 90, or 100 amino acid differences thereto, or a nucleic acid (e.g., DNA or mRNA) encoding the gene modifying polypeptide, and
(b) a template RNA (or DNA encoding the template RNA) comprising (i) a sequence that binds the polypeptide and (ii) a heterologous object sequence.

In some embodiments, the method is performed ex vivo or in vitro. In some embodiments, the gene modifying polypeptide and/or template RNA are formulated with an LNP.

In some embodiments, contacting the cell with the system results in insertion of the heterologous object sequence into at least 1%, 5%, 10%, 15%, 20%, 25%, 30%, 40%, 50%, 60%, or 70% of respiratory epithelial cells (e.g., bronchial epithelial cells, e.g., human bronchial epithelial (hBE) cells).

Additionally provided herein is a reaction mixture comprising a gene modifying system disclosed herein (e.g., comprising a heterologous object sequence) and one or more respiratory epithelial cells (e.g., bronchial epithelial cells, e.g., human bronchial epithelial (hBE) cells).

In some embodiments, the reaction mixture comprises a gene modifying polypeptide, or a nucleic acid (e.g., DNA or mRNA) encoding the gene modifying polypeptide, and a respiratory epithelial cell (e.g., a bronchial epithelial cell, e.g., a human bronchial epithelial (hBE) cell). In some embodiments, the reaction mixture further comprises a template RNA (or DNA encoding the template RNA) comprising (i) a sequence that binds the polypeptide and (ii) a heterologous object sequence.

In some embodiments, the reaction mixture comprises a gene modifying polypeptide comprising an amino acid sequence of Table R1 or E14 or a sequence at least 70%, 75%, 80%, 85%, 90%, 95%, 96%, 97%, 98%, or 99% identical thereto, or a nucleic acid (e.g., DNA or mRNA) encoding the gene modifying polypeptide, and a respiratory epithelial cell (e.g., a bronchial epithelial cell, e.g., a human bronchial epithelial (hBE) cell). In some embodiments, the reaction mixture additionally includes a template RNA (or DNA encoding the template RNA) comprising (i) a sequence that binds the polypeptide and (ii) a heterologous object sequence.

In some embodiments, the reaction mixture comprises a gene modifying polypeptide comprising an amino acid sequence of Table R1 or E14 or a sequence no more than 10, 20, 30, 40, 50, 60, 70, 80, 90, or 100 amino acid differences thereto, or a nucleic acid (e.g., DNA or mRNA) encoding the gene modifying polypeptide, and a respiratory epithelial cell (e.g., a bronchial epithelial cell, e.g., a human bronchial epithelial (hBE) cell). In some embodiments, the reaction mixture additionally includes a template RNA (or DNA encoding the template RNA) comprising (i) a sequence that binds the polypeptide and (ii) a heterologous object sequence.

In some embodiments, modifying a genome of a cell (e.g., a primary cell, e.g., a T cell or an IPSC) using a gene modifying system does not result in activation of the endogenous DNA damage response (DDR) pathway. In some embodiments, modifying a genome of a cell (e.g., a primary cell, e.g., an IPSC) using a gene modifying system results in activation of the cell's endogenous DDR pathway less than in an otherwise similar cell treated with Cas9, e.g., in an assay according to Example 2.

In some embodiments, modifying a genome of a cell (e.g., a primary cell, e.g., a T cell or an IPSC) using a gene modifying system does not result in activation of the endogenous interferon response. In some embodiments, modifying a genome of a cell (e.g., a primary cell, e.g., a T cell or an IPSC) using a gene modifying system results in activation of the cell's interferon response less than in an otherwise similar cell treated with a gene modifying system comprising elements from a LINE-1 retrotransposase, e.g., in an assay according to Example 3.

### Suitable Indications

Exemplary suitable diseases and disorders that can be treated by the systems or methods provided herein, for example, those comprising gene modifying systems, include, without limitation: Baraitser-Winter syndromes 1 and 2; Diabetes mellitus and insipidus with optic atrophy and deafness; Alpha-1-antitrypsin deficiency; Heparin cofactor II deficiency; Adrenoleukodystrophy; Keppen-Lubinsky syndrome; Treacher collins syndrome 1; Mitochondrial complex I, II, III, III (nuclear type 2, 4, or 8) deficiency; Hypermanganesemia with dystonia, polycythemia and cirrhosis; Carcinoid tumor of intestine; Rhabdoid tumor predisposition syndrome 2; Wilson disease; Hyperphenylalaninemia, bh4-deficient, a, due to partial pts deficiency, BH4-deficient, D, and non-pku; Hyperinsulinemic hypoglycemia familial 3, 4, and 5; Keratosis follicularis; Oral-facial-digital syndrome; SeSAME syndrome; Deafness, nonsyndromic sensorineural, mitochondrial; Proteinuria; Insulin-dependent diabetes mellitus secretory diarrhea syndrome; Moyamoya disease 5; Diamond-Blackfan anemia 1, 5, 8, and 10; Pseudoachondroplastic spondyloepiphyseal dysplasia syndrome; Brittle cornea syndrome 2; Methylmalonic acidemia with homocystinuria; Adams-Oliver syndrome 5 and 6; autosomal recessive Agammaglobulinemia 2; Cortical malformations, occipital; Febrile seizures, familial, 11; Mucopolysaccharidosis type VI, type VI (severe), and type VII; Marden Walker like syndrome; Pseudoneonatal adrenoleukodystrophy; Spheroid body myopathy; Cleidocranial dysostosis; Multiple Cutaneous and Mucosal Venous Malformations; Liver failure acute infantile; Neonatal intrahepatic cholestasis caused by citrin deficiency; Ventricular septal defect 1; Oculodentodigital dysplasia; Wilms tumor 1; Weill-Marchesani-like syndrome; Renal adysplasia; Cataract 1, 4, autosomal dominant, autosomal dominant, multiple types, with microcornea, coppock-like, juvenile, with microcornea and glucosuria, and nuclear diffuse nonprogressive; Odontohypophosphatasia; Cerebro-oculo-facio-skeletal syndrome; Schizophrenia 15; Cerebral amyloid angiopathy, APP-related; Hemophagocytic lymphohistiocytosis, familial, 3; Porphobilinogen synthase deficiency; Episodic ataxia type 2; Trichorhinophalangeal syndrome type 3; Progressive familial heart block type IB; Glioma susceptibility 1; Lichtenstein-Knorr Syndrome; Hypohidrotic X-linked ectodermal dysplasia; Bartter syndrome types 3, 3 with hypocalciuria, and 4; Carbonic anhydrase VA deficiency, hyperammonemia due to; Cardiomyopathy; Poikiloderma, hereditary fibrosing, with tendon contractures, myopathy, and pulmonary fibrosis; Combined d-2- and 1-2-hydroxyglutaric aciduria; Arginase deficiency; Cone-rod dystrophy 2 and 6; Smith-Lemli-Opitz syndrome; Mucolipidosis III Gamma; Blau syndrome; Werner syndrome; Meningioma; Iodotyrosyl coupling defect; Dubin-Johnson syndrome; 3-Oxo-5 alpha-steroid delta 4-dehydrogenase deficiency; Boucher Neuhauser syndrome; Iron accumulation in brain; Mental Retardation, X-Linked 102 and syndromic 13; familial, Pituitary adenoma predisposition; Hypoplasia of the corpus callosum; Hyperalphalipoproteinemia 2; Deficiency of ferroxidase; Growth hormone insensitivity with immunodeficiency; Marinesco-Sj\xc3\xb6gren syndrome; Martsolf syndrome; Gaze palsy, familial horizontal, with progressive scoliosis; Mitchell-Riley syndrome; Hypocalciuric hypercalcemia, familial, types 1 and 3; Rubinstein-Taybi syndrome; Epstein syndrome; Juvenile retinoschisis; Becker muscular dystrophy; Loeys-Dietz syndrome 1, 2, 3; Congenital muscular hypertrophy-cerebral syndrome; Familial juvenile gout; Spermatogenic failure 11, 3, and 8; Orofacial cleft 11 and 7, Cleft lip/palate-ectodermal dysplasia syndrome; Mental retardation, X-linked, nonspecific, syndromic, Hedera type, and syndromic, wu type; Combined oxidative phosphorylation deficiencies 1, 3, 4, 12, 15, and 25; Frontotemporal dementia; Kniest dysplasia; Familial cardiomyopathy; Benign familial hematuria; Pheochromocytoma; Aminoglycoside-induced deafness; Gamma-aminobutyric acid transaminase deficiency; Oculocutaneous albinism type IB, type 3, and type 4; Renal coloboma syndrome; CNS hypomyelination; Hennekam lymphangiectasia-lymphedema syndrome 2; Migraine, familial basilar; Distal spinal muscular atrophy, X-linked 3; X-linked periventricular heterotopia; Microcephaly; Mucopolysaccharidosis, MPS-I-H/S, MPS-II, MPS-III-A, MPS-III-B, MPS-III-C, MPS-IV-A, MPS-IV-B; Infantile Parkinsonism-dystonia; Frontotemporal dementia with TDP43 inclusions, TARDBP-related; Hereditary diffuse gastric cancer; Sialidosis type I and II; Microcephaly-capillary malformation syndrome; Hereditary breast and ovarian cancer syndrome; Brain small vessel disease with hemorrhage; Non-ketotic hyperglycinemia; Navajo neurohepatopathy; Auriculocondylar syndrome 2; Spastic paraplegia 15, 2, 3, 35, 39, 4, autosomal dominant, 55, autosomal recessive, and 5A; Autosomal recessive cutis laxa type IA and IB; Hemolytic anemia, nonspherocytic, due to glucose phosphate isomerase deficiency; Hutchinson-Gilford syndrome; Familial amyloid nephropathy with urticaria and deafness; Supravalvar aortic stenosis; Diffuse palmoplantar keratoderma, Bothnian type; Holt-Oram syndrome; Coffin Siris/Intellectual Disability; Left-right axis malformations; Rapadilino syndrome; Nanophthalmos 2; Craniosynostosis and dental anomalies; Paragangliomas 1; Snyder Robinson syndrome; Ventricular fibrillation; Activated PI3K-delta syndrome; Howel-Evans syndrome; Larsen syndrome, dominant type; Van Maldergem syndrome 2; MYH-associated polyposis; 6-pymvoyl-tetrahydropterin synthase deficiency; Alagille syndromes 1 and 2; Lymphangiomyomatosis; Muscle eye brain disease; WFS1-Related Disorders; Primary hypertrophic osteoarthropathy, autosomal recessive 2; Infertility; Nestor-Guillermo progeria syndrome; Mitochondrial trifunctional protein deficiency; Hypoplastic left heart syndrome 2; Primary dilated cardiomyopathy; Retinitis pigmentosa; Hirschsprung disease 3; Upshaw-Schulman syndrome; Desbuquois dysplasia 2; Diarrhea 3 (secretory sodium, congenital, syndromic) and 5 (with tufting enteropathy, congenital); Pachyonychia congenita 4 and type 2; Cerebral autosomal dominant and recessive arteriopathy with subcortical infarcts and leukoencephalopathy; Vi tel 1i form dystrophy; type II, type IV, IV (combined hepatic and myopathic), type V, and type VI; Atypical Rett syndrome; Atrioventricular septal defect 4; Papillon-Lef\xc3\xa8vre syndrome; Leber amaurosis; X-linked hereditary motor and sensory neuropathy; Progressive sclerosing poliodystrophy; Goldmann-Favre syndrome; Renal-hepatic-pancreatic dysplasia; Pallister-Hall syndrome; Amyloidogenic transthyretin amyloidosis; Melnick-Needles syndrome; Hyperimmunoglobulin E syndrome; Posterior column ataxia with retinitis pigmentosa; Chondrodysplasia punctata 1, X-linked recessive and 2 X-linked dominant; Ectopia lentis, isolated autosomal recessive and dominant; Familial cold urticarial; Familial adenomatous polyposis 1 and 3; Porokeratosis 8, disseminated superficial actinic type; PIK3CA Related Overgrowth Spectrum; Cerebral cavernous malformations 2; Exudative vitreoretinopathy 6; Megalencephaly cutis marmorata telangiectatica congenital; TARP syndrome; Diabetes mellitus, permanent neonatal, with neurologic features; Short-rib thoracic dysplasia 11 or 3 with or without polydactyly; Hypertrichotic osteochondrodysplasia; beta Thalassemia; Niemann-Pick disease type C1, C2, type A, and type C1, adult form; Charcot-Marie-Tooth disease types IB, 2B2, 2C, 2F, 21, 2U (axonal), 1C (demyelinating), dominant intermediate C, recessive intermediate A, 2A2, 4C, 4D, 4H, IF, IVF, and X; Tyrosinemia type I; Paroxysmal atrial fibrillation; UV-sensitive syndrome; Tooth agenesis, selective, 3 and 4; Merosin deficient congenital muscular dystrophy; Long-chain 3-hydroxyacyl-CoA dehydrogenase deficiency; Congenital aniridia; Left ventricular noncompaction 5; Deficiency of aromatic-L-amino-acid decarboxylase; Coronary heart disease; Leukonychia totalis; Distal arthrogryposis type 2B; Retinitis pigmentosa 10, 11, 12, 14, 15, 17, and 19; Robinow Sorauf syndrome; Tenorio Syndrome; Prolactinoma; Neurofibromatosis, type land type 2; Congenital muscular dystrophy-dystroglycanopathy with brain and eye anomalies, types A2, A7, A8, A11, and A14; Heterotaxy, visceral, 2, 4, and 6, autosomal; Jankovic Rivera syndrome; Lipodystrophy, familial partial, type 2 and 3; Hemoglobin H disease, nondeletional; Multicentric osteolysis, nodulosis and arthropathy; Thyroid agenesis; deficiency of Acyl-CoA dehydrogenase family, member 9; Alexander disease; Phytanic acid storage disease; Breast-ovarian cancer, familial 1, 2, and 4; Proline dehydrogenase deficiency; Childhood hypophosphatasia; Pancreatic agenesis and congenital heart disease; Vitamin D-dependent rickets, types land 2; Iridogoniodysgenesis dominant type and type 1; Autosomal recessive hypohidrotic ectodermal dysplasia syndrome; Mental retardation, X-linked, 3, 21, 30, and 72; Hereditary hemorrhagic telangiectasia type 2; Blepharophimosis, ptosis, and epicanthus inversus; Adenine phosphoribosyltransferase deficiency; Seizures, benign familial infantile, 2; Acrodysostosis 2, with or without hormone resistance; Tetralogy of Fallot; Retinitis pigmentosa 2, 20, 25, 35, 36, 38, 39, 4, 40, 43, 45, 48, 66, 7, 70, 72; Lysosomal acid lipase deficiency; Eichsfeld type congenital muscular dystrophy; Walker-Warburg congenital muscular dystrophy; TNF receptor-associated periodic fever syndrome (TRAPS); Progressive myoclonus epilepsy with ataxia; Epilepsy, childhood absence 2, 12 (idiopathic generalized, susceptibility to) 5 (nocturnal frontal lobe), nocturnal frontal lobe type 1, partial, with variable foci, progressive myoclonic 3, and X-linked, with variable learning disabilities and behavior disorders; Long QT syndrome; Dicarboxylic aminoaciduria; Brachydactyly types A1 and A2; Pseudoxanthoma elasticum-like disorder with multiple coagulation factor deficiency; Multisystemic smooth muscle dysfunction syndrome; Syndactyly Cenani Lenz type; Joubert syndrome 1, 6, 7, 9/15 (digenic), 14, 16, and 17, and Orofaciodigital syndrome xiv; Digitorenocerebral syndrome; Retinoblastoma; Dyskinesia, familial, with facial myokymia; Hereditary sensory and autonomic neuropathy type IIB and IIA; familial hyperinsulinism; Megalencephalic leukoencephalopathy with subcortical cysts land 2a; Aase syndrome; Wiedemann-Steiner syndrome; Ichthyosis exfoliativa; Myotonia congenital; Granulomatous disease, chronic, X-linked, variant; Deficiency of 2-methylbutyryl-CoA dehydrogenase; Sarcoidosis, early-onset; Glaucoma, congenital and Glaucoma, congenital, Coloboma; Breast cancer, susceptibility to; Ceroid lipofuscinosis neuronal 2, 6, 7, and 10; Congenital generalized lipodystrophy type 2; Fructose-biphosphatase deficiency; Congenital contractural arachnodactyly; Lynch syndrome I and II; Phosphoglycerate dehydrogenase deficiency; Burn-Mckeown syndrome; Myocardial infarction 1; Achromatopsia 2 and 7; Retinitis Pigmentosa 73; Protan defect; Polymicrogyria, asymmetric, bilateral frontoparietal; Spinal muscular atrophy, distal, autosomal recessive, 5; Methylmalonic aciduria due to methylmalonyl-CoA mutase deficiency; Familial porencephaly; Hurler syndrome; Oto-palato-digital syndrome, types I and II; Sotos syndrome 1 or 2; Cardioencephalomyopathy, fatal infantile, due to cytochrome c oxidase deficiency; Parastremmatic dwarfism; Thyrotropin releasing hormone resistance, generalized; Diabetes mellitus, type 2, and insulin-dependent, 20; Thoracic aortic aneurysms and aortic dissections; Estrogen resistance; Maple syrup urine disease type 1A and type 3; Hypospadias 1 and 2, X-linked; Metachromatic leukodystrophy juvenile, late infantile, and adult types; Early T cell progenitor acute lymphoblastic leukemia; Neuropathy, Hereditary Sensory, Type IC; Mental retardation, autosomal dominant 31; Retinitis pigmentosa 39; Breast cancer, early-onset; May-Hegglin anomaly; Gaucher disease type 1 and Subacute neuronopathic; Temtamy syndrome; Spinal muscular atrophy, lower extremity predominant 2, autosomal dominant; Fanconi anemia, complementation group E, I, N, and O; Alkaptonuria; Hirschsprung disease; Combined malonic and methylmalonic aciduria; Arrhythmogenic right ventricular cardiomyopathy types 5, 8, and 10; Congenital lipomatous overgrowth, vascular malformations, and epidermal nevi; Timothy syndrome; Deficiency of guanidinoacetate methyltransferase; Myoclonic dystonia; Kanzaki disease; Neutral 1 amino acid transport defect; Neurohypophyseal diabetes insipidus; Thyroid hormone metabolism, abnormal; Benign scapuloperoneal muscular dystrophy with cardiomyopathy; Hypoglycemia with deficiency of glycogen synthetase in the liver; Hypertrophic cardiomyopathy; Myasthenic Syndrome, Congenital, 11, associated with acetylcholine receptor deficiency; Mental retardation X-linked syndromic 5; Stormorken syndrome; Aplastic anemia; Intellectual disability; Normokalemic periodic paralysis, potassium-sensitive; Danon disease; Nephronophthisis 13, 15 and 4; Thyrotoxic periodic paralysis and Thyrotoxic periodic paralysis 2; Infertility associated with multi-tailed spermatozoa and excessive DNA; Glaucoma, primary open angle, juvenile-onset; Afibrinogenemia and congenital Afibrinogenemia; Polycystic kidney disease 2, adult type, and infantile type; Familial porphyria cutanea tarda; Cerebello-oculo-renal syndrome (nephronophthisis, oculomotor apraxia and cerebellar abnormalities); Frontotemporal Dementia Chromosome 3-Linked and Frontotemporal dementia ubiquitin-positive; Metatrophic dysplasia; Immunodeficiency-centromeric instability-facial anomalies syndrome 2; Anemia, nonspherocytic hemolytic, due to G6PD deficiency; Bronchiectasis with or without elevated sweat chloride 3; Congenital myopathy with fiber type disproportion; Carney complex, type 1; Cryptorchidism, unilateral or bilateral; Ichthyosis bullosa of Siemens; Isolated lutropin deficiency; DFNA 2 Nonsyndromic Hearing Loss; Klein-Waardenberg syndrome; Gray platelet syndrome; Bile acid synthesis defect, congenital, 2; 46, XY sex reversal, type 1, 3, and 5; Acute intermittent porphyria; Cornelia de Fange syndromes 1 and 5; Hyperglycinuria; Cone-rod dystrophy 3; Dysfibrinogenemia; Karak syndrome; Congenital muscular dystrophy-dystroglycanopathy without mental retardation, type B5; Infantile nystagmus, X-linked; Dyskeratosis congenita, autosomal recessive, 1, 3, 4, and 5; Microcephaly with or without chorioretinopathy, lymphedema, or mental retardation; Hyperlysinemia; Bardet-Biedl syndromes 1, 11, 16, and 19; Autosomal recessive centronuclear myopathy; Frasier syndrome; Caudal regression syndrome; Fibrosis of extraocular muscles, congenital, 1, 2, 3a (with or without extraocular involvement), 3b; Prader-Willi-like syndrome; Malignant melanoma; Bloom syndrome; Darier disease, segmental; Multicentric osteolysis nephropathy; Hemochromatosis type 1, 2B, and 3; Cerebellar ataxia infantile with progressive external ophthalmoplegi and Cerebellar ataxia, mental retardation, and dysequilibrium syndrome 2; Hypoplastic left heart syndrome; Epilepsy, Hearing Loss, And Mental Retardation Syndrome; Transferrin serum level quantitative trait locus 2; Ocular albinism, type I; Marfan syndrome; Congenital muscular dystrophy-dystroglycanopathy with brain and eye anomalies, type A14 and B14; Hyperammonemia, type III; Cryptophthalmos syndrome; Alopecia universalis congenital; Adult hypophosphatasia; Mannose-binding protein deficiency; Bull eye macular dystrophy; Autosomal dominant torsion dystonia 4; Nephrotic syndrome, type 3, type 5, with or without ocular abnormalities, type 7, and type 9; Seizures, Early infantile epileptic encephalopathy 7; Persistent hyperinsulinemic hypoglycemia of infancy; Thrombocytopenia, X-linked; Neonatal hypotonia; Orstavik Lindemann Solberg syndrome; Pulmonary hypertension, primary, 1, with hereditary hemorrhagic telangiectasia; Pituitary dependent hypercortisolism; Epidermodysplasia verruciformis; Epidermolysis bullosa, junctional, localisata variant; Cytochrome c oxidase i deficiency; Kindler syndrome; Myosclerosis, autosomal recessive; Truncus arteriosus; Duane syndrome type 2; ADULT syndrome; Zellweger syndrome spectrum; Leukoencephalopathy with ataxia, with Brainstem and Spinal Cord Involvement and Lactate Elevation, with vanishing white matter, and progressive, with ovarian failure; Antithrombin III deficiency; Holoprosencephaly 7; Roberts-SC phocomelia syndrome; Mitochondrial DNA-depletion syndrome 3 and 7, hepatocerebral types, and 13 (encephalomyopathic type); Porencephaly 2; Microcephaly, normal intelligence and immunodeficiency; Giant axonal neuropathy; Sturge-Weber syndrome, Capillary malformations, congenital, 1; Fabry disease and Fabry disease, cardiac variant; Glutamate formiminotransferase deficiency; Fanconi-Bickel syndrome; Acromicric dysplasia; Epilepsy, idiopathic generalized, susceptibility to, 12, Basal ganglia calcification, idiopathic, 4; Polyglucosan body myopathy 1 with or without immunodeficiency; Malignant tumor of prostate; Congenital ectodermal dysplasia of face; Congenital heart disease; Age-related macular degeneration 3, 6, 11, and 12; Congenital myotonia, autosomal dominant and recessive forms; Hypomagnesemia 1, intestinal; Sulfite oxidase deficiency, isolated; Pick disease; Plasminogen deficiency, type I; Syndactyly type 3; Cone-rod dystrophy amelogenesis imperfecta; Pseudoprimary hyperaldosteronism; Terminal osseous dysplasia; Bartter syndrome antenatal type 2; Congenital muscular dystrophy-dystroglycanopathy with mental retardation, types B2, B3, B5, and B15; Familial infantile myasthenia; Lymphoproliferative syndrome 1, 1 (X-linked), and 2; Hypercholesterolaemia and Hypercholesterolemia, autosomal recessive; Neoplasm of ovary; Infantile GM1 gangliosidosis; Syndromic X-linked mental retardation 16; Deficiency of ribose-5-phosphate isomerase; Alzheimer disease, types, 1, 3, and 4; Andersen Tawil syndrome; Multiple synostoses syndrome 3; Chilbain lupus 1; Hemophagocytic lymphohistiocytosis, familial, 2; Axenfeld-Rieger syndrome type 3; Myopathy, congenital with cores; Osteoarthritis with mild chondrodysplasia; Peroxisome biogenesis disorders; Severe congenital neutropenia; Hereditary neuralgic amyotrophy; Palmoplantar keratoderma, nonepidermolytic, focal or diffuse; Dysplasminogenemia; Familial colorectal cancer; Spastic ataxia 5, autosomal recessive, Charlevoix-Saguenay type, 1, 10, or 11, autosomal recessive; Frontometaphyseal dysplasia land 3; Hereditary factors II, IX, VIII deficiency disease; Spondylocheirodysplasia, Ehlers-Danlos syndrome-like, with immune dysregulation, Aggrecan type, with congenital joint dislocations, short limb-hand type, Sedaghatian type, with cone-rod dystrophy, and Kozlowski type; Ichthyosis prematurity syndrome; Stickler syndrome type 1; Focal segmental glomerulosclerosis 5; 5-Oxoprolinase deficiency; Microphthalmia syndromic 5, 7, and 9; Juvenile polyposis/hereditary hemorrhagic telangiectasia syndrome; Deficiency of butyryl-CoA dehydrogenase; Maturity-onset diabetes of the young, type 2; Mental retardation, syndromic, Claes-Jensen type, X-linked; Deafness, cochlear, with myopia and intellectual impairment, without vestibular involvement, autosomal dominant, X-linked 2; Spondylocarpotarsal synostosis syndrome; Sting-associated vasculopathy, infantile-onset; Neutral lipid storage disease with myopathy; Immune dysfunction with T-cell inactivation due to calcium entry defect 2; Cardiofaciocutaneous syndrome; Corticosterone methyloxidase type 2 deficiency; Hereditary myopathy with early respiratory failure; Interstitial nephritis, karyomegalic; Trimethylaminuria; Hyperimmunoglobulin D with periodic fever; Malignant hyperthermia susceptibility type 1; Trichomegaly with mental retardation, dwarfism and pigmentary degeneration of retina; Breast adenocarcinoma; Complement factor B deficiency; Ullrich congenital muscular dystrophy; Left ventricular noncompaction cardiomyopathy; Fish-eye disease; Finnish congenital nephrotic syndrome; Limb-girdle muscular dystrophy, type IB, 2A, 2B, 2D, C1, C5, C9, C14; Idiopathic fibrosing alveolitis, chronic form; Primary familial hypertrophic cardiomyopathy; Angiotensin i-converting enzyme, benign serum increase; Cd8 deficiency, familial; Proteus syndrome; Glucose-6-phosphate transport defect; Borjeson-Forssman-Lehmann syndrome; Zellweger syndrome; Spinal muscular atrophy, type II; Prostate cancer, hereditary, 2; Thrombocytopenia, platelet dysfunction, hemolysis, and imbalanced globin synthesis; Congenital disorder of glycosylation types IB, ID, IG, 1H, 1 J, IK, IN, IP, 2C, 2J, 2K, Ilm; Junctional epidermolysis bullosa gravis of Herlitz; Generalized epilepsy with febrile seizures plus 3, type 1, type 2; Schizophrenia 4; Coronary artery disease, autosomal dominant 2; Dyskeratosis congenita, autosomal dominant, 2 and 5; Subcortical laminar heterotopia, X-linked; Adenylate kinase deficiency; X-linked severe combined immunodeficiency; Coproporphyria; Amyloid Cardiomyopathy, Transthyretin-related; Hypocalcemia, autosomal dominant 1; Brugada syndrome; Congenital myasthenic syndrome, acetazolamide-responsive; Primary hypomagnesemia; Sclerosteosis; Frontotemporal dementia and/or amyotrophic lateral sclerosis 3 and 4; Mevalonic aciduria; Schwannomatosis 2; Hereditary motor and sensory neuropathy with optic atrophy; Porphyria cutanea tarda; Osteochondritis dissecans; Seizures, benign familial neonatal, 1, and/or myokymia; Long QT syndrome, LQT1 subtype; Mental retardation, anterior maxillary protrusion, and strabismus; Idiopathic hypercalcemia of infancy; Hypogonadotropic hypogonadism 11 with or without anosmia; Polycystic lipomembranous osteodysplasia with sclerosing leukoencephalopathy; Primary autosomal recessive microcephaly 10, 2, 3, and 5; Interrupted aortic arch; Congenital amegakaryocytic thrombocytopenia; Hermansky-Pudlak syndrome 1, 3, 4, and 6; Long QT syndrome 1, 2, 2/9, 2/5, (digenic), 3, 5 and 5, acquired, susceptibility to; Andermann syndrome; Retinal cone dystrophy 3B; Erythropoietic protoporphyria; Sepiapterin reductase deficiency; Very long chain acyl-CoA dehydrogenase deficiency; Hyperferritinemia cataract syndrome; Silver spastic paraplegia syndrome; Charcot-Marie-Tooth disease; Atrial septal defect 2; Carnevale syndrome; Hereditary insensitivity to pain with anhidrosis; Catecholaminergic polymorphic ventricular tachycardia; Hypokalemic periodic paralysis 1 and 2; Sudden infant death syndrome; Hypochromic microcytic anemia with iron overload; GLUT1 deficiency syndrome 2; Leukodystrophy, Hypomyelinating, 11 and 6; Cone monochromatism; Osteopetrosis autosomal dominant type 1 and 2, recessive 4, recessive 1, recessive 6; Severe congenital neutropenia 3, autosomal recessive or dominant; Methionine adenosyltransferase deficiency, autosomal dominant; Paroxysmal familial ventricular fibrillation; Pyruvate kinase deficiency of red cells; Schneckenbecken dysplasia; Torsades de pointes; Distal myopathy Markesbery-Griggs type; Deficiency of UDPglucose-hexose-1-phosphate uridylyltransferase; Sudden cardiac death; Neu-Laxova syndrome 1; Atransferrinemia; Hyperparathyroidism 1 and 2; Cutaneous malignant melanoma 1; Symphalangism, proximal, lb; Progressive pseudorheumatoid dysplasia; Werdnig-Hoffmann disease; Achondrogenesis type 2; Holoprosencephaly 2, 3, 7, and 9; Schindler disease, type 1; Cerebroretinal microangiopathy with calcifications and cysts; Heterotaxy, visceral, X-linked; Tuberous sclerosis syndrome; Kartagener syndrome; Thyroid hormone resistance, generalized, autosomal dominant; Bestrophinopathy, autosomal recessive; Nail disorder, nonsyndromic congenital, 8; Mohr-Tranebjaerg syndrome; Cone-rod dystrophy 12; Hearing impairment; Ovarioleukodystrophy; Renal tubular acidosis, proximal, with ocular abnormalities and mental retardation; Dihydropteridine reductase deficiency; Focal epilepsy with speech disorder with or without mental retardation; Ataxia-telangiectasia syndrome; Brown-Vialetto-Van laere syndrome and Brown-Vialetto-Van Laere syndrome 2; Cardiomyopathy; Peripheral demyelinating neuropathy, central dysmyelination; Corneal dystrophy, Fuchs endothelial, 4; Cowden syndrome 3; Dystonia 2 (torsion, autosomal recessive), 3 (torsion, X-linked), 5 (Dopa-responsive type), 10, 12, 16, 25, 26 (Myoclonic); Epiphyseal dysplasia, multiple, with myopia and conductive deafness; Cardiac conduction defect, nonspecific; Branchiootic syndromes 2 and 3; Peroxisome biogenesis disorder 14B, 2A, 4A, 5B, 6A, 7A, and 7B; Familial renal glucosuria; Candidiasis, familial, 2, 5, 6, and 8; Autoimmune disease, multisystem, infantile-onset; Early infantile epileptic encephalopathy 2, 4, 7, 9, 10, 11, 13, and 14; Segawa syndrome, autosomal recessive; Deafness, autosomal dominant 3a, 4, 12, 13, 15, autosomal dominant nonsyndromic sensorineural 17, 20, and 65; Congenital dyserythropoietic anemia, type I and II; Enhanced s-cone syndrome; Adult neuronal ceroid lipofuscinosis; Atrial fibrillation, familial, 11, 12, 13, and 16; Norum disease; Osteosarcoma; Partial albinism; Biotinidase deficiency; Combined cellular and humoral immune defects with granulomas; Alpers encephalopathy; Holocarboxylase synthetase deficiency; Maturity-onset diabetes of the young, type 1, type 2, type 11, type 3, and type 9; Variegate porphyria; Infantile cortical hyperostosis; Testosterone 17-beta-dehydrogenase deficiency; L-2-hydroxyglutaric aciduria; Tyrosinase-negative oculocutaneous albinism; Primary ciliary dyskinesia 24; Pontocerebellar hypoplasia type 4; Ciliary dyskinesia, primary, 7, 11, 15, 20 and 22; Idiopathic basal ganglia calcification 5; Brain atrophy; Craniosynostosis 1 and 4; Keratoconus 1; Rasopathy; Congenital adrenal hyperplasia and Congenital adrenal hypoplasia, X-linked; Mitochondrial DNA depletion syndrome 11, 12 (cardiomyopathic type), 2, 4B (MNGIE type), 8B (MNGIE type); Brachydactyly with hypertension; Cornea plana 2; Aarskog syndrome; Multiple epiphyseal dysplasia 5 or Dominant; Corneal endothelial dystrophy type 2; Aminoacylase 1 deficiency; Delayed speech and language development; Nicolaides-Baraitser syndrome; Enterokinase deficiency; Ectrodactyly, ectodermal dysplasia, and cleft lip/palate syndrome 3; Arthrogryposis multiplex congenita, distal, X-linked; Perrault syndrome 4; Jervell and Lange-Nielsen syndrome 2; Hereditary Nonpolyposis Colorectal Neoplasms; Robinow syndrome, autosomal recessive, autosomal recessive, with brachy-syn-polydactyly; Neurofibrosarcoma; Cytochrome-c oxidase deficiency; Vesicoureteral reflux 8; Dopamine beta hydroxylase deficiency; Carbohydrate-deficient glycoprotein syndrome type I and II; Progressive familial intrahepatic cholestasis 3; Benign familial neonatal-infantile seizures; Pancreatitis, chronic, susceptibility to; Rhizomelic chondrodysplasia punctata type 2 and type 3; Disordered steroidogenesis due to cytochrome p450 oxidoreductase deficiency; Deafness with labyrinthine aplasia microtia and microdontia (FAMM); Rothmund-Thomson syndrome; Cortical dysplasia, complex, with other brain malformations 5 and 6; Myasthenia, familial infantile, 1; Trichorhinophalangeal dysplasia type I; Worth disease; Splenic hypoplasia; Molybdenum cofactor deficiency, complementation group A; Sebastian syndrome; Progressive familial intrahepatic cholestasis 2 and 3; Weill-Marchesani syndrome 1 and 3; Microcephalic osteodysplastic primordial dwarfism type 2; Surfactant metabolism dysfunction, pulmonary, 2 and 3; Severe X-linked myotubular myopathy; Pancreatic cancer 3; Platelet-type bleeding disorder 15 and 8; Tyrosinase-positive oculocutaneous albinism; Borrone Di Rocco Crovato syndrome; ATR-X syndrome; Sucrase-isomaltase deficiency; Complement component 4, partial deficiency of, due to dysfunctional cl inhibitor; Congenital central hypoventilation; Infantile hypophosphatasia; Plasminogen activator inhibitor type 1 deficiency; Malignant lymphoma, non-Hodgkin; Hyperornithinemia-hyperammonemia-homocitrullinuria syndrome; Schwartz Jampel syndrome type 1; Fetal hemoglobin quantitative trait locus 1; Myopathy, distal, with anterior tibial onset; Noonan syndrome 1 and 4, LEOPARD syndrome 1; Glaucoma 1, open angle, e, F, and G; Kenny-Caffey syndrome type 2; PTEN hamartoma tumor syndrome; Duchenne muscular dystrophy; Insulin-resistant diabetes mellitus and acanthosis nigricans; Microphthalmia, isolated 3, 5, 6, 8, and with coloboma 6; Raine syndrome; Premature ovarian failure 4, 5, 7, and 9; Allan-Hemdon-Dudley syndrome; Citrullinemia type I; Alzheimer disease, familial, 3, with spastic paraparesis and apraxia; Familial hemiplegic migraine types 1 and 2; Ventriculomegaly with cystic kidney disease; Pseudoxanthoma elasticum; Homocysteinemia due to MTHFR deficiency, CBS deficiency, and Homocystinuria, pyridoxine-responsive; Dilated cardiomyopathy 1A, 1AA, IC, IG, IBB, 1DD, IFF, 1HH, II, IKK, IN, IS, 1Y, and 3B; Muscle AMP guanine oxidase deficiency; Familial cancer of breast; Hereditary sideroblastic anemia; Myoglobinuria, acute recurrent, autosomal recessive; Neuroferritinopathy; Cardiac arrhythmia; Glucose transporter type 1 deficiency syndrome; Holoprosencephaly sequence; Angiopathy, hereditary, with nephropathy, aneurysms, and muscle cramps; Isovaleryl-CoA dehydrogenase deficiency; Kallmann syndrome 1, 2, and 6; Permanent neonatal diabetes mellitus; Acrocallosal syndrome, Schinzel type; Gordon syndrome; MYH9 related disorders; Donnai Barrow syndrome; Severe congenital neutropenia and 6, autosomal recessive; Charcot-Marie-Tooth disease, types ID and IVF; Coffin-Lowry syndrome; mitochondrial 3-hydroxy-3-methylglutaryl-CoA synthase deficiency; Hypomagnesemia, seizures, and mental retardation; Ischiopatellar dysplasia; Multiple congenital anomalies—hypotonia—seizures syndrome 3; Spastic paraplegia 50, autosomal recessive; Short stature with nonspecific skeletal abnormalities; Severe myoclonic epilepsy in infancy; Propionic academia; Adolescent nephronophthisis; Macrocephaly, macrosomia, facial dysmorphism syndrome; Stargardt disease 4; Ehlers-Danlos syndrome type 7 (autosomal recessive), classic type, type 2 (progeroid), hydroxylysine-deficient, type 4, type 4 variant, and due to tenascin-X deficiency; Myopia 6; Coxa plana; Familial cold autoinflammatory syndrome 2; Malformation of the heart and great vessels; von Willebrand disease type 2M and type 3; Deficiency of galactokinase; Brugada syndrome 1; X-linked ichthyosis with steryl-sulfatase deficiency; Congenital ocular coloboma; Histiocytosis-lymphadenopathy plus syndrome; Aniridia, cerebellar ataxia, and mental retardation; Left ventricular noncompaction 3; Amyotrophic lateral sclerosis types 1, 6, 15 (with or without frontotemporal dementia), 22 (with or without frontotemporal dementia), and 10; Osteogenesis imperfecta type 12, type 5, type 7, type 8, type I, type III, with normal sclerae, dominant form, recessive perinatal lethal; Hematologic neoplasm; Favism, susceptibility to; Pulmonary Fibrosis And/Or Bone Marrow Failure, Telomere-Related, 1 and 3; Dominant hereditary optic atrophy; Dominant dystrophic epidermolysis bullosa with absence of skin; Muscular dystrophy, congenital, megaconial type; Multiple gastrointestinal atresias; McCune-Albright syndrome; Nail-patella syndrome; McLeod neuroacanthocytosis syndrome; Common variable immunodeficiency 9; Partial hypoxanthine-guanine phosphoribosyltransferase deficiency; Pseudohypoaldosteronism type 1 autosomal dominant and recessive and type 2; Urocanate hydratase deficiency; Heterotopia; Meckel syndrome type 7; Ch\xc3\xa9diak-Higashi syndrome, Chediak-Higashi syndrome, adult type; Severe combined immunodeficiency due to ADA deficiency, with microcephaly, growth retardation, and sensitivity to ionizing radiation, atypical, autosomal recessive, T cell-negative, B cell-positive, NK cell-negative of NK-positive; Insulin resistance; Deficiency of steroid 11-beta-monooxygenase; Popliteal pterygium syndrome; Pulmonary arterial hypertension related to hereditary hemorrhagic telangiectasia; Deafness, autosomal recessive 1A, 2, 3, 6, 8, 9, 12, 15, 16, 18b, 22, 28, 31, 44, 49, 63, 77, 86, and 89; Primary hyperoxaluria, type I, type, and type III; Paramyotonia congenita of von Eulenburg; Desbuquois syndrome; Carnitine palmitoyltransferase I, II, II (late onset), and II (infantile) deficiency; Secondary hypothyroidism; Mandibulofacial dysostosis, Treacher Collins type, autosomal recessive; Cowden syndrome 1; Li-Fraumeni syndrome 1; Asparagine synthetase deficiency; Malattia leventinese; Optic atrophy 9; Infantile convulsions and paroxysmal choreoathetosis, familial; Ataxia with vitamin E deficiency; Islet cell hyperplasia; Miyoshi muscular dystrophy 1; Thrombophilia, hereditary, due to protein C deficiency, autosomal dominant and recessive; Fechtner syndrome; Properdin deficiency, X-linked; Mental retardation, stereotypic movements, epilepsy, and/or cerebral malformations; Creatine deficiency, X-linked; Pilomatrixoma; Cyanosis, transient neonatal and atypical nephropathic; Adult onset ataxia with oculomotor apraxia; Hemangioma, capillary infantile; PC-K6a; Generalized dominant dystrophic epidermolysis bullosa; Pelizaeus-Merzbacher disease; Myopathy, centronuclear, 1, congenital, with excess of muscle spindles, distal, 1, lactic acidosis, and sideroblastic anemia 1, mitochondrial progressive with congenital cataract, hearing loss, and developmental delay, and tubular aggregate, 2; Benign familial neonatal seizures 1 and 2; Primary pulmonary hypertension; Lymphedema, primary, with myelodysplasia; Congenital long QT syndrome; Familial exudative vitreoretinopathy, X-linked; Autosomal dominant hypohidrotic ectodermal dysplasia; Primordial dwarfism; Familial pulmonary capillary hemangiomatosis; Carnitine acylcamitine translocase deficiency; Visceral myopathy; Familial Mediterranean fever and Familial mediterranean fever, autosomal dominant; Combined partial and complete 17-alpha-hydroxylase/17, 20-lyase deficiency; Oto-palato-digital syndrome, type I; Nephrolithiasis/osteoporosis, hypophosphatemic, 2; Familial type 1 and 3 hyperlipoproteinemia; Phenotypes; CHARGE association; Fuhrmann syndrome; Hypotrichosis-lymphedema-telangiectasia syndrome; Chondrodysplasia Blomstrand type; Acroerythrokeratoderma; Slowed nerve conduction velocity, autosomal dominant; Hereditary cancer-predisposing syndrome; Craniodiaphyseal dysplasia, autosomal dominant; Spinocerebellar ataxia autosomal recessive 1 and 16; Proprotein convertase 1/3 deficiency; D-2-hydroxyglutaric aciduria 2; Hyperekplexia 2 and Hyperekplexia hereditary; Central core disease; Opitz G/BBB syndrome; Cystic fibrosis; Thiel-Behnke corneal dystrophy; Deficiency of bisphosphoglycerate mutase; Mitochondrial short-chain Enoyl-CoA Hydratase 1 deficiency; Ectodermal dysplasia skin fragility syndrome; Wolfram-like syndrome, autosomal dominant; Microcytic anemia; Pyruvate carboxylase deficiency; Leukocyte adhesion deficiency type I and III; Multiple endocrine neoplasia, types land 4; Transient bullous dermolysis of the newborn; Primrose syndrome; Non-small cell lung cancer; Congenital muscular dystrophy; Lipase deficiency combined; COLE-CARPENTER SYNDROME 2; Atrioventricular septal defect and common atrioventricular junction; Deficiency of xanthine oxidase; Waardenburg syndrome type 1, 4C, and 2E (with neurologic involvement); Stickler syndrome, types 1 (nonsyndromic ocular) and 4; Corneal fragility keratoglobus, blue sclerae and joint hypermobility; Microspherophakia; Chudley-McCullough syndrome; Epidermolysa bullosa simplex and limb girdle muscular dystrophy, simplex with mottled pigmentation, simplex with pyloric atresia, simplex, autosomal recessive, and with pyloric atresia; Rett disorder; Abnormality of neuronal migration; Growth hormone deficiency with pituitary anomalies; Leigh disease; Keratosis palmoplantaris striata 1; Weissenbacher-Zweymuller syndrome; Medium-chain acyl-coenzyme A dehydrogenase deficiency; UDPglucose-4-epimerase deficiency; susceptibility to Autism, X-linked 3; Rhegmatogenous retinal detachment, autosomal dominant; Familial febrile seizures 8; Ulna and fibula absence of with severe limb deficiency; Left ventricular noncompaction 6; Centromeric instability of chromosomes 1, 9 and 16 and immunodeficiency; Hereditary diffuse leukoencephalopathy with spheroids; Cushing syndrome; Dopamine receptor d2, reduced brain density of; C-like syndrome; Renal dysplasia, retinal pigmentary dystrophy, cerebellar ataxia and skeletal dysplasia; Ovarian dysgenesis 1; Pierson syndrome; Polyneuropathy, hearing loss, ataxia, retinitis pigmentosa, and cataract; Progressive intrahepatic cholestasis; autosomal dominant, autosomal recessive, and X-linked recessive Alport syndromes; Angelman syndrome; Amish infantile epilepsy syndrome; Autoimmune lymphoproliferative syndrome, type 1a; Hydrocephalus; Marfanoid habitus; Bare lymphocyte syndrome type 2, complementation group E; Recessive dystrophic epidermolysis bullosa; Factor H, VII, X, v and factor viii, combined deficiency of 2, xiii, a subunit, deficiency; Zonular pulverulent cataract 3; Warts, hypogammaglobulinemia, infections, and myelokathexis; Benign hereditary chorea; Deficiency of hyaluronoglucosaminidase; Microcephaly, hiatal hernia and nephrotic syndrome; Growth and mental retardation, mandibulofacial dysostosis, microcephaly, and cleft palate; Lymphedema, hereditary, id; Delayed puberty; Apparent mineralocorticoid excess; Generalized arterial calcification of infancy 2; METHYLMALONIC ACIDURIA, mut(0) TYPE; Congenital heart disease, multiple types, 2; Familial hypoplastic, glomerulocystic kidney; Cerebrooculofacioskeletal syndrome 2; Stargardt disease 1; Mental retardation, autosomal recessive 15, 44, 46, and 5; Prolidase deficiency; Methylmalonic aciduria cblB type; Oguchi disease; Endocrine-cerebroosteodysplasia; Lissencephaly 1, 2 (X-linked), 3, 6 (with microcephaly), X-linked; Somatotroph adenoma; Gamstorp-Wohlfart syndrome; Lipid proteinosis; Inclusion body myopathy 2 and 3; Enlarged vestibular aqueduct syndrome; Osteoporosis with pseudoglioma; Acquired long QT syandrome; Phenylketonuria; CHOPS syndrome; Global developmental delay; Bietti crystalline corneoretinal dystrophy; Noonan syndrome-like disorder with or without juvenile myelomonocytic leukemia; Congenital erythropoietic porphyria; Atrophiabulborum hereditaria; Paragangliomas 3; Van der Woude syndrome; Aromatase deficiency; Birk Barel mental retardation dysmorphism syndrome; Amyotrophic lateral sclerosis type 5; Methemoglobinemia types I 1 and 2; Congenital stationary night blindness, type 1A, IB, 1C, IE, IF, and 2A; Seizures; Thyroid cancer, follicular; Lethal congenital contracture syndrome 6; Distal hereditary motor neuronopathy type 2B; Sex cord-stromal tumor; Epileptic encephalopathy, childhood-onset, early infantile, 1, 19, 23, 25, 30, and 32; Myofibrillar myopathy 1 and ZASP-related; Cerebellar ataxia infantile with progressive external ophthalmoplegia; Purine-nucleoside phosphorylase deficiency; Forebrain defects; Epileptic encephalopathy Lennox-Gastaut type; Obesity; 4, Left ventricular noncompaction 10; Verheij syndrome; Mowat-Wilson syndrome; Odontotrichomelic syndrome; Patterned dystrophy of retinal pigment epithelium; Lig4 syndrome; Barakat syndrome; IRAK4 deficiency; Somatotroph adenoma; Branched-chain ketoacid dehydrogenase kinase deficiency; Cystinuria; Familial aplasia of the vermis; Succinyl-CoA acetoacetate transferase deficiency; Scapuloperoneal spinal muscular atrophy; Pigmentary retinal dystrophy; Glanzmann thrombasthenia; Primary open angle glaucoma juvenile onset 1; Aicardi Goutieres syndromes 1, 4, and 5; Renal dysplasia; Intrauterine growth retardation, metaphyseal dysplasia, adrenal hypoplasia congenita, and genital anomalies; Beaded hair; Short stature, onychodysplasia, facial dysmorphism, and hypotrichosis; Metachromatic leukodystrophy; Cholestanol storage disease; Three M syndrome 2; Leber congenital amaurosis 11, 12, 13, 16, 4, 7, and 9; Mandibuloacral dysplasia with type A or B lipodystrophy, atypical; Meier-Gorlin syndromes land 4; Hypotrichosis 8 and 12; Short QT syndrome 3; Ectodermal dysplasia 1 ib; Anonychia; Pseudohypoparathyroidism type 1A, Pseudopseudohypoparathyroidism; Leber optic atrophy; Bainbridge-Ropers syndrome; Weaver syndrome; Short stature, auditory canal atresia, mandibular hypoplasia, skeletal abnormalities; Deficiency of alpha-mannosidase; Macular dystrophy, vitelliform, adult-onset; Glutaric aciduria, type 1; Gangliosidosis GM1 typel (with cardiac involvement) 3; Mandibuloacral dysostosis; Hereditary lymphedema type I; Atrial standstill 2; Kabuki make-up syndrome; Bethlem myopathy and Bethlem myopathy 2; Myeloperoxidase deficiency; Fleck corneal dystrophy; Hereditary acrodermatitis enteropathica; Hypobetalipoproteinemia, familial, associated with apob32; Cockayne syndrome type A; Hyperparathyroidism, neonatal severe; Ataxia-telangiectasia-like disorder; Pendred syndrome; I blood group system; Familial benign pemphigus; Visceral heterotaxy 5, autosomal; Nephrogenic diabetes insipidus, Nephrogenic diabetes insipidus, X-linked; Minicore myopathy with external ophthalmoplegia; Perry syndrome; hypohidrotic/hair/tooth type, autosomal recessive; Hereditary pancreatitis; Mental retardation and microcephaly with pontine and cerebellar hypoplasia; Glycogen storage disease 0 (muscle), II (adult form), IXa2, IXc, type 1A; Osteopathia striata with cranial sclerosis; Gluthathione synthetase deficiency; Brugada syndrome and Brugada syndrome 4; Endometrial carcinoma; Hypohidrotic ectodermal dysplasia with immune deficiency; Cholestasis, intrahepatic, of pregnancy 3; Bemard-Soulier syndrome, types A1 and A2 (autosomal dominant); Salla disease; Ornithine aminotransferase deficiency; PTEN hamartoma tumor syndrome; Distichiasis-lymphedema syndrome; Corticosteroid-binding globulin deficiency; Adult neuronal ceroid lipofuscinosis; Dejerine-Sottas disease; Tetraamelia, autosomal recessive; Senior-Loken syndrome 4 and 5; Glutaric acidemia IIA and IIB; Aortic aneurysm, familial thoracic 4, 6, and 9; Hyperphosphatasia with mental retardation syndrome 2, 3, and 4; Dyskeratosis congenita X-linked; Arthrogryposis, renal dysfunction, and cholestasis 2; Bannayan-Riley-Ruvalcaba syndrome; 3-Methylglutaconic aciduria; Isolated 17,20-lyase deficiency; Gorlin syndrome; Hand foot uterus syndrome; Tay-Sachs disease, B1 variant, Gm2-gangliosidosis (adult), Gm2-gangliosidosis (adult-onset); Dowling-degos disease 4; Parkinson disease 14, 15, 19 (juvenile-onset), 2, 20 (early-onset), 6, (autosomal recessive early-onset, and 9; Ataxia, sensory, autosomal dominant; Congenital microvillous atrophy; Myoclonic-Atonic Epilepsy; Tangier disease; 2-methyl-3-hydroxybutyric aciduria; Familial renal hypouricemia; Schizencephaly; Mitochondrial DNA depletion syndrome 4B, MNGIE type; Feingold syndrome 1; Renal carnitine transport defect; Familial hypercholesterolemia; Townes-Brocks-branchiootorenal-like syndrome; Griscelli syndrome type 3; Meckel-Gruber syndrome; Bullous ichthyosiform erythroderma; Neutrophil immunodeficiency syndrome; Myasthenic Syndrome, Congenital, 17, 2A (slow-channel), 4B (fast-channel), and without tubular aggregates; Microvascular complications of diabetes 7; McKusick Kaufman syndrome; Chronic granulomatous disease, autosomal recessive cytochrome b-positive, types 1 and 2; Arginino succinate lyase deficiency; Mitochondrial phosphate carrier and pyruvate carrier deficiency; Lattice corneal dystrophy Type III; Ectodermal dysplasia-syndactyly syndrome 1; Hypomyelinating leukodystrophy 7; Mental retardation, autosomal dominant 12, 13, 15, 24, 3, 30, 4, 5, 6, and 9; Generalized epilepsy with febrile seizures plus, types 1 and 2; Psoriasis susceptibility 2; Frank Ter Haar syndrome; Thoracic aortic aneurysms and aortic dissections; Crouzon syndrome; Granulosa cell tumor of the ovary; Epidermolytic palmoplantar keratoderma; Leri Weill dyschondrosteosis; 3 beta-Hydroxysteroid dehydrogenase deficiency; Familial restrictive cardiomyopathy 1; Autosomal dominant progressive external ophthalmoplegia with mitochondrial DNA deletions 1 and 3; Antley-Bixler syndrome with genital anomalies and disordered steroidogenesis; Hajdu-Cheney syndrome; Pigmented nodular adrenocortical disease, primary, 1; Episodic pain syndrome, familial, 3; Dejerine-Sottas syndrome, autosomal dominant; FG syndrome and FG syndrome 4; Dendritic cell, monocyte, B lymphocyte, and natural killer lymphocyte deficiency; Hypothyroidism, congenital, nongoitrous, 1; Miller syndrome; Nemaline myopathy 3 and 9; Oligodontia-colorectal cancer syndrome; Cold-induced sweating syndrome 1; Van Buchem disease type 2; Glaucoma 3, primary congenital, d; Citrullinemia type I and II; Nonaka myopathy; Congenital muscular dystrophy due to partial LAMA2 deficiency; Myoneural gastrointestinal encephalopathy syndrome; Leigh syndrome due to mitochondrial complex I deficiency; Medulloblastoma; Pyruvate dehydrogenase E1-alpha deficiency; Carcinoma of colon; Nance-Horan syndrome; Sandhoff disease, adult and infantil types; Arthrogryposis renal dysfunction cholestasis syndrome; Autosomal recessive hypophosphatemic bone disease; Doyne honeycomb retinal dystrophy; Spinocerebellar ataxia 14, 21, 35, 40, and 6; Lewy body dementia; RRM2B-related mitochondrial disease; Brody myopathy; Megalencephaly-polymicrogyria-polydactyly-hydrocephalus syndrome 2; Usher syndrome, types 1, IB, ID, 1G, 2A, 2C, and 2D; hypocalcification type and hypomaturation type, IIA1 Amelogenesis imperfecta; Pituitary hormone deficiency, combined 1, 2, 3, and 4; Cushing symphalangism; Renal tubular acidosis, distal, autosomal recessive, with late-onset sensorineural hearing loss, or with hemolytic anemia; Infantile nephronophthisis; Juvenile polyposis syndrome; Sensory ataxic neuropathy, dysarthria, and ophthalmoparesis; Deficiency of 3-hydroxyacyl-CoA dehydrogenase; Parathyroid carcinoma; X-linked agammaglobulinemia; Megaloblastic anemia, thiamine-responsive, with diabetes mellitus and sensorineural deafness; Multiple sulfatase deficiency; Neurodegeneration with brain iron accumulation 4 and 6; Cholesterol monooxygenase (side-chain cleaving) deficiency; hemolytic anemia due to Adenylosuccinate lyase deficiency; Myoclonus with epilepsy with ragged red fibers; Pitt-Hopkins syndrome; Multiple pterygium syndrome Escobar type; Homocystinuria-Megaloblastic anemia due to defect in cobalamin metabolism, cblE complementation type; Cholecystitis; Spherocytosis types 4 and 5; Multiple congenital anomalies; Xeroderma pigmentosum, complementation group b, group D, group E, and group G; Leiner disease; Groenouw corneal dystrophy type 1; Coenzyme Q10 deficiency, primary 1, 4, and 7; Distal spinal muscular atrophy, congenital nonprogressive; Warburg micro syndrome 2 and 4; Bile acid synthesis defect, congenital, 3; Acth-independent macronodular adrenal hyperplasia 2; Acrocapitofemoral dysplasia; Paget disease of bone, familial; Severe neonatal-onset encephalopathy with microcephaly; Zimmermann-Laband syndrome and Zimmermann-Laband syndrome 2; Reifenstein syndrome; Familial hypokalemia-hypomagnesemia; Photosensitive trichothiodystrophy; Adult junctional epidermolysis bullosa; Lung cancer; Freeman-Sheldon syndrome; Hyperinsulinism-hyperammonemia syndrome; Posterior polar cataract type 2; Sclerocornea, autosomal recessive; Juvenile GM>1<gangliosidosis; Cohen syndrome; Hereditary Paraganglioma-Pheochromocytoma Syndromes; Neonatal insulin-dependent diabetes mellitus; Hypochondrogenesis; Floating-Harbor syndrome; Cutis laxa with osteodystrophy and with severe pulmonary, gastrointestinal, and urinary abnormalities; Congenital contractures of the limbs and face, hypotonia, and developmental delay; Dyskeratosis congenita autosomal dominant and autosomal dominant, 3; Histiocytic medullary reticulosis; Costello syndrome; Immunodeficiency 15, 16, 19, 30, 31C, 38, 40, 8, due to defect in cd3-zeta, with hyper IgM type 1 and 2, and X-Linked, with magnesium defect, Epstein-Barr vims infection, and neoplasia; Atrial septal defects 2, 4, and 7 (with or without atrioventricular conduction defects); GTP cyclohydrolase I deficiency; Talipes equinovarus; Phosphoglycerate kinase 1 deficiency; Tuberous sclerosis 1 and 2; Autosomal recessive congenital ichthyosis 1, 2, 3, 4A, and 4B; and Familial hypertrophic cardiomyopathy 1, 2, 3, 4, 7, 10, 23 and 24.

### Indications by Tissue

Additional suitable diseases and disorders that can be treated by the systems and methods provided herein include, without limitation, diseases of the central nervous system (CNS) (see exemplary diseases and affected genes in Table 13), diseases of the eye (see exemplary diseases and affected genes in Table 14), diseases of the heart (see exemplary diseases and affected genes in Table 15), diseases of the hematopoietic stem cells (HSC) (see exemplary diseases and affected genes in Table 16), diseases of the kidney (see exemplary diseases and affected genes in Table 17), diseases of the liver (see exemplary diseases and affected genes in Table 18), diseases of the lung (see exemplary diseases and affected genes in Table 19), diseases of the skeletal muscle (see exemplary diseases and affected genes in Table 20), and diseases of the skin (see exemplary diseases and affected genes in Table 21). Table 22 provides exemplary protective mutations that reduce risks of the indicated diseases. In some embodiments, a gene modifying system described herein is used to treat an indication of any of Tables 13-21. In some embodiments, the GeneWriter system modifies a target site in genomic DNA in a cell, wherein the target site is in a gene of any of Tables 13-21, e.g., in a subject having the corresponding indication listed in any of Tables 13-21. In some embodiments, the gene modifying systems corrects a mutation in the gene. In some embodiments, the gene modifying system inserts a sequence that had been deleted from the gene (e.g., through a disease-causing mutation). In some embodiments, the gene modifying system deletes a sequence that had been duplicated in the gene (e.g., through a disease-causing mutation). In some embodiments, the gene modifying system replaces a mutation (e.g., a disease-causing mutation) with the corresponding wild-type sequence. In some embodiments, the mutation is a substitution, insertion, deletion, or inversion.

TABLE 13
CNS diseases and genes affected.

| | Gene |
|---|---|
| Disease | Affected |
| Alpha-mannosidosis | MAN2B1 |
| Ataxia-telangiectasia | ATM |
| CADASIL | NOTCH3 |
| Canavan disease | ASPA |
| Carbamoyl-phosphate synthetase 1 deficiency | CPS1 |
| CLN1 disease | PPT1 |
| CLN2 Disease | TPP1 |
| CLN3 Disease (Juvenile neuronal ceroid | CLN3 |
| lipofuscinosis, Batten Disease) | |
| Coffin-Lowry syndrome | RPS6KA3 |
| Congenital myasthenic syndrome 5 | COLQ |
| Cornelia de Lange syndrome (NIPBL) | NIPBL |
| Cornelia de Lange syndrome (SMC1A) | SMC1A |
| Dravet syndrome (SCN1A) | SCN1A |
| Glycine encephalopathy (GLDC) | GLDC |
| GM1 gangliosidosis | GLB1 |
| Huntington's Disease | HTT |
| Hydrocephalus with stenosis of the aqueduct of | L1CAM |
| Sylvius | |
| Leigh Syndrome | SURF1 |
| Metachromatic leukodystrophy (ARSA) | ARSA |
| MPS type 2 | IDS |
| MPS type 3 | Type 3a: |
| | SGSH |
| | Type 3b: |
| | NAGLU |
| Mucolipidosis IV | MCOLN1 |
| Neurofibromatosis Type 1 | NF1 |
| Neurofibromatosis type 2 | NF2 |
| Pantothenate kinase-associated neurodegeneration | PANK2 |
| Pyridoxine-dependent epilepsy | ALDH7A1 |
| Rett syndrome (MECP2) | MECP2 |
| Sandhoff disease | HEXB |
| Semantic dementia (Frontotemporal dementia) | MAPT |
| Spinocerebellar ataxia with axonal neuropathy | SETX |
| (Ataxia with Oculomotor Apraxia) | |
| Tay-Sachs disease | HEXA |
| X-linked Adrenoleukodystrophy | ABCD1 |

TABLE 14
Eye diseases and genes affected.

| Disease | Gene Affected |
|---|---|
| Achromatopsia | CNGB3 |
| Amaurosis Congenita (LCA1) | GUCY2D |
| Amaurosis Congenita (LCA10) | CEP290 |
| Amaurosis Congenita (LCA2) | RPE65 |
| Amaurosis Congenita (LCA8) | CRB1 |
| Choroideremia | CHM |
| Cone Rod Dystrophy (ABCA4) | ABCA4 |
| Cone Rod Dystrophy (CRX) | CRX |
| Cone Rod Dystrophy (GUCY2D) | GUCY2D |
| Cystinosis, Ocular Nonnephropathic | CTNS |
| Lattice corneal dystrophy type I | TGFBI |
| Macular Corneal Dystrophy (MCD) | CHST6 |
| Optic Atrophy | OPA1 |
| Retinitis Pigmentosa (AR) | USH2A |
| Retinitis Rigmentosa (AD) | RHO |
| Stargardt Disease | ABCA4 |
| Vitelliform Macular Dystrophy | BEST1; PRPH2 |

TABLE 15
Heart diseases and genes affected.

| | Gene |
|---|---|
| Disease | Affected |
| Arrhythmogenic right ventricular cardiomyopathy (ARVC) | PKP2 |
| Barth syndrome | TAZ |
| Becker muscular dystrophy | DMD |
| Brugada syndrome | SCN5A |
| Catecholaminergic polymorphic ventricular tachycardia | RYR2 |
| (RYR2) | |
| Dilated cardiomyopathy (LMNA) | LMNA |
| Dilated cardiomyopathy (TTN) | TTN |
| Duchenne muscular dystrophy | DMD |
| Emery-Dreifuss Muscular Dystrophy Type I | EMD |
| Familial hypertrophic cardiomyopathy | MYH7 |
| Familial hypertrophic cardiomyopathy | MYBPC3 |
| Jervell Lange-Nielsen syndrome | KCNQ1 |
| LCHAD deficiency | HADHA |
| Limb-girdle muscular dystrophy type 1B (Emery-Dreifuss | LMNA |
| EDMD2) | |
| Limb-girdle muscular dystrophy, type 2D | SGCA |
| Long QT syndrome 1 (Romano Ward) | KCNQ1 |

TABLE 16
HSC diseases and genes affected.

| Disease | Gene Affected |
|---|---|
| ADA-SCID | ADA |
| Adrenoleukodystrophy (CALD) | ABCD1 |
| Alpha-mannosidosis | MAN2B1 |
| Chronic granulomatous disease | CYBB; CYBA; NCF1; |
| | NCF2; NCF4 |
| Common variable immunodeficiency | TNFRSF13B |
| Fanconi anemia | FANCA; FANCC; FANCG |
| Gaucher disease | GBA |
| Globoid cell leukodystrophy (Krabbe | GALC |
| disease) | |
| Hemophagocytic lymphohistiocytosis | PRF1; STX11; STXBP2; |
| | UNC13D |
| IL-7R SCID | IL7R |
| JAK-3 SCID | JAK3 |
| Malignant infantile osteopetrosis- | TCIRG1; Many genes |
| autosomal recessive osteopetrosis | implicated |
| Metachromatic leukodystrophy | ARSA; PSAP |
| MPS 1S (Scheie syndrome) | IDUA |
| MPS2 | IDS |
| MPS7 | GUSB |
| Mucolipidosis II | GNPTAB |
| Niemann-Pick disease A and B | SMPD1 |
| Niemann-Pick disease C | NPC1 |
| Paroxysmal Nocturnal Hemoglobinuria | PIGA |
| Pompe disease | GAA |
| Pyruvate kinase deficiency (PKD) | PKLR |
| RAG 1/2 Deficiency (SCID with | RAG1/RAG2 |
| granulomas) | |
| Severe Congenital Neutropenia | ELANE; HAX1 |
| Sickle cell disease (SCD) | HBB |
| Tay Sachs | HEXA |
| Thalassemia | HBB |
| Wiskott-Aldrich Syndrome | WAS |
| X-linked agammaglobulinemia | BTK |
| X-linked SCID | IL2RG |

TABLE 17
Kidney diseases and genes affected.

| | Gene |
|---|---|
| Disease | Affected |
| Alport syndrome | COL4A5 |
| Autosomal dominant polycystic kidney disease (PKD1) | PKD1 |
| Autosomal dominant polycystic kidney disease (PKD2) | PDK2 |
| Autosomal dominant tubulointerstitial kidney disease | MUC1 |
| (MUC1) | |
| Autosomal dominant tubulointerstitial kidney disease | UMOD |
| (UMOD) | |
| Autosomal recessive polycystic kidney disease | PKHD1 |
| Congenital nephrotic syndrome | NPHS2 |
| Cystinosis | CTNS |

TABLE 18
Liver diseases and genes affected.

| Disease | Gene Affected |
|---|---|
| Acute intermittent porphyria | HMBS |
| Alagille syndrome | JAG1 |
| Alpha-1-antitrypsin deficiency | SERPINA1 |
| Carbamoyl phosphate synthetase I | CPS1 |
| deficiency | |
| Citrullinemia I | ASS1 |
| Crigler-Najjar | UGT1A1 |
| Fabry | LPL |
| Familial chylomicronemia syndrome | GLA |
| Gaucher | GBE1 |
| GSD IV | GBA |
| Heme A | F8 |
| Heme B | F9 |
| Hereditary amyloidosis (hTTR) | TTR |
| Hereditary angioedema | SERPING1 (KLKB1 |
| | for CRISPR) |
| HoFH | LDLRAP1 |
| Hypercholesterolemia | PCSK9 |
| Methylmalonic acidemia | MMUT |
| MPS II | IDS |
| MPS III | Type IIIa: SGSH |
| | Type IIIb: NAGLU |
| | Type IIIc: HGSNAT |
| | Type IIId: GNS |
| MPS IV | Type IVA: GALNS |
| | Type IVB: GLB1 |
| MPS VI | ARSB |
| MSUD | Type Ia: BCKDHA |
| | Type Ib: BCKDHB |
| | Type II: DBT |
| OTC Deficiency | OTC |
| Polycystic Liver Disease | PRKCSH |
| Pompe | GAA |
| Primary Hyperoxaluria 1 | AGXT (HAO1 or LDHA |
| | for CRISPR) |
| Progressive familial intrahepatic | ATP8B1 |
| cholestasis type 1 | |
| Progressive familial intrahepatic | ABCB11 |
| cholestasis type 2 | |
| Progressive familial intrahepatic | ABCB4 |
| cholestasis type 3 | |
| Propionic acidemia | PCCB; PCCA |
| Wilson's Disease | ATP7B |
| Glycogen storage disease, Type 1a | G6PC |
| Glycogen storage disease, Type IIIb | AGL |
| Isovaleric acidemia | IVD |
| Wolman disease | LIPA |

TABLE 19
Lung diseases and genes affected.

| Disease | Gene Affected |
|---|---|
| Alpha-1 antitrypsin deficiency | SERPINA1 |
| Cystic fibrosis | CFTR |
| Primary ciliary dyskinesia | DNAI1 |
| Primary ciliary dyskinesia | DNAH5 |
| Primary pulmonary hypertension I | BMPR2 |
| Surfactant Protein B (SP-B) Deficiency (pulmonary | SFTPB |
| surfactant metabolism dysfunction 1) | |

TABLE 20
Skeletal muscle diseases and genes affected.

| Disease | Gene Affected |
|---|---|
| Becker muscular dystrophy | DMD |
| Becker myotonia | CLCN1 |
| Bethlem myopathy | COL6A2 |
| Centronuclear myopathy, X-linked | MTM1 |
| (myotubular) | |
| Congenital myasthenic syndrome | CHRNE |
| Duchenne muscular dystrophy | DMD |
| Emery-Dreifuss muscular dystrophy, AD | LMNA |
| Facioscapulohumeral Muscular Dystrophy | DUX4 - D4Z4 |
| | chromosomal region |
| Hyperkalemic periodic paralysis | SCN4A |
| Hypokalemic periodic paralysis | CACNA1S |
| Limb-girdle muscular dystrophy 2A | CAPN3 |
| Limb-girdle muscular dystrophy 2B | DYSF |
| Limb-girdle muscular dystrophy, type 2D | SGCA |
| Miyoshi muscular dystrophy 1 | DYSF |
| Paramyotonia congenita | SCN4A |
| Thomsen myotonia | CLCN1 |
| VCP myopathy (IBMPFD) 1 | VCP |

TABLE 21
Skin diseases and genes affected.

| Disease | Gene Affected |
|---|---|
| Epidermolysis Bullosa Dystrophica Dominant | COL7A1 |
| Epidermolysis Bullosa Dystrophica Recessive | COL7A1 |
| (Hallopeau-Siemens) | |
| Epidermolysis Bullosa Junctional | LAMB3 |
| Epidermolysis Bullosa Simplex | KRT5; KRT14 |
| Epidermolytic Ichthyosis | KRT1; KRT10 |
| Hailey-Hailey Disease | ATP2C1 |
| Lamellar Ichthyosis/Nonbullous Congenital | TGM1 |
| Ichthyosiform Erythroderma (ARCI) | |
| Netherton Syndrome | SPINK5 |

TABLE 22
Exemplary protective mutations that reduce disease risk.

| Disease | Gene | Exemplary Protective Mutation |
|---|---|---|
| Alzheimer's | APP | A673T |
| Parkinson's | SGK1 | |
| Diabetes (Type II) | SLC30A8 | p.Arg138X; p.Lys34SerfsX50 |
| Cardiovascular | PCSK9 | R46L |
| Disease | | |
| Cardiovascular | ASGR1 | NM_001671.4, c.284 − 36_283 + 33delCTGGGGCTGGGG; |
| Disease | | (SEQ ID NO: 15433) NP_001662.1, p.W158X |
| Cardiovascular | NPC1L1 | p.Arg406X |
| Disease | | |
| Cardiovascular | APOC3 | R19X; IVS2 + 1G→A; A43T |
| Disease | | |
| Cardiovascular | LPA | |
| Disease | | |
| Cardiovascular | ANGPTL4 | E40K |
| Disease | | |
| Cardiovascular | ANGPTL3 | p.Ser17Ter; p.Asn121fs; p.Asn147fs; c.495 + 6T→C |
| Disease | | |
| HIV infection | CCR5 | CCR5-delta32 |

### Pathogenic Mutations

In some embodiments, the systems or methods provided herein can be used to correct a pathogenic mutation. The pathogenic mutation can be a genetic mutation that increases an individual's susceptibility or predisposition to a certain disease or disorder. In some embodiments, the pathogenic mutation is a disease-causing mutation in a gene associated with a disease or disorder. In some embodiments, the systems or methods provided herein can be used to revert the pathogenic mutation to its wild-type counterpart. In some embodiments, the systems or methods provided herein can be used to change the pathogenic mutation to a sequence not causing the disease or disorder.

Table 23 provides exemplary indications (column 1), underlying genes (column 2), and pathogenic mutations that can be corrected using the systems or methods described herein (column 3).

TABLE 23
Indications, genes, and causitive pathogenic mutations.

| Disease | Gene | Pathogenic Mutation^{#} |
|---|---|---|
| Achromatopsia | CNGB3 | 1148delC |
| Alpha-1 Antitrypsin Deficiency | SERPINAI | E342K |
| Alpha-1 Antitrypsin Deficiency | SERPINAI | E342K |
| Alpha-1 Antitrypsin Deficiency | SERPINAI | R48C (R79C) |
| Amaurosis Congenita (LCA10) | CEP290 | 2991 + 1655A > G |
| Andersen- Tawil syndrome | KCNJ2 | R218W |
| Arrhythmogenic right ventricular | PKP2 | c.235C > T |
| cardiomyopathy (ARVC) | | |
| associated with congenital factor XI deficiency | F11 | E117* |
| associated with congenital factor XI deficiency | F11 | F283L |
| ATTR amyloidosis | TTR | V50M/N30M |
| autosomal dominant deafness | COCH | G88E |
| autosomal dominant deafness | TECTA | Y1870C |
| autosomal dominant Parkinson's disease | SNCA | A53T |
| autosomal dominant Parkinson's disease | SNCA | A30P |
| Autosomal dominant rickets | FGF23 | R176Q |
| autosomal recessive deafness | CX30 | T5M |
| autosomal recessive deafness | DFNB59 | R183W |
| autosomal recessive deafness | TMC1 | Y182C |
| autosomal recessive hypercholesterolemia | ARH | Q136* |
| Blackfan-Diamond anemia | RPS19 | R62Q |
| blue-cone monochromatism | OPN1LW | C203R |
| Brugada syndrome | SCN5A | E1784K |
| CADASIL syndrome | NOTCH3 gene | R90C |
| CADASIL syndrome | NOTCH3 gene | R141C |
| Canavan disease | ASPA | E285A |
| Canavan disease | ASPA | Y231X |
| Canavan disease | ASPA | A305E |
| carnitine palmitoyltransferase II deficiency | CPT2 | S113L |
| choroideremia | CHM | R293* |
| choroideremia | CHM | R270* |
| choroideremia | CHM | A117A |
| Citrullinemia Type I | ASS | G390R |
| classic galactosemia | GALT | Q188R |
| classic homocysteinuria | CBS | T191M |
| classic homocysteinuria | CBS | G307S |
| CLN2 Disease | TPP1 | c.509 − 1 G > C |
| CLN2 Disease | TPP1 | c.622 C > T |
| CLN2 Disease | TPP1 | c.851 G > T |
| cone-rod dystrophy | GUCY2D | R838C |
| congenital factor V deficiency | F5 | R506Q |
| congenital factor V deficiency | F5 | R534Q |
| congenital factor VII deficiency | F7 | A294V |
| congenital factor VII deficiency | F7 | C310F |
| congenital factor VII deficiency | F7 | R304Q |
| congenital factor VII deficiency | F7 | QI00R |
| Creutzfeldt- Jakob disease (CJD) | PRNP | E200K |
| Creutzfeldt- Jakob disease (CJD) | PRNP | M129V |
| Creutzfeldt- Jakob disease (CJD) | PRNP | P102L |
| Creutzfeldt- Jakob disease (CJD) | PRNP | D178N |
| cystic fibrosis | CFTR | G551D |
| cystic fibrosis | CFTR | W1282* |
| cystic fibrosis | CFTR | R553* |
| cystic fibrosis | CFTR | R117H |
| cystic fibrosis | CFTR | delta F508 |
| cystinosis | CTNS | W138* |
| Darier disease | ATP2A2 | N767S |
| Darier disease | ATP2A2 | N767S |
| Darier disease | ATP2A2 | N767S |
| Epidermolysis Bullosa Junctional | LAMB3 | R42X |
| Epidermolysis Bullosa Junctional | LAMB3 | R635X |
| familial amyotrophic lateral sclerosis (ALS) | SOD1 | A4V |
| familial amyotrophic lateral sclerosis (ALS) | SOD1 | H46R |
| familial amyotrophic lateral sclerosis (ALS) | SOD1 | G37R |
| Gaucher disease | GBA | N370S |
| Gaucher disease | GBA | N370S |
| Gaucher disease | GBA | L444P |
| Gaucher disease | GBA | L444P |
| Gaucher disease | GBA | L483P |
| glutaryl-CoA dehydrogenase deficiency | GCDH | R138G |
| glutaryl-CoA dehydrogenase deficiency | GCDH | M263V |
| glutaryl-CoA dehydrogenase deficiency | GCDH | R402W |
| glycine encephalopathy | GLDC | A389V |
| glycine encephalopathy | GLDC | G771R |
| glycine encephalopathy | GLDC | T269M |
| hemophilia A | F8 | R2178C |
| hemophilia A | F8 | R550C |
| hemophilia A | F8 | R2169H |
| hemophilia A | F8 | R1985Q |
| hemophilia B | F9 | T342M |
| hemophilia B | F9 | R294Q |
| hemophilia B | F9 | R43Q |
| hemophilia B | F9 | R191H |
| hemophilia B | F9 | G106S |
| hemophilia B | F9 | A279T |
| hemophilia B | F9 | R75* |
| hemophilia B | F9 | R294* |
| hemophilia B | F9 | R379Q |
| Hereditary antithrombin deficiency type I | SERPINCI | R48C (R79C) |
| hereditary chronic pancreatitis | PRSS1 | R122H |
| Hunter syndrome | IDS | R88C |
| Hunter syndrome | IDS | G374G |
| Hurler syndrome (MPS1) | IDUA | Q70* |
| Hurler syndrome (MPS1) | IDUA | W402* |
| Hyperkalemic periodic paralysis | SCN4A | T704M |
| Hyperkalemic periodic paralysis | SCN4A | M1592V |
| Hyperkalemic periodic paralysis | CACNA1S | p.Arg528X |
| Hyperkalemic periodic paralysis | CACNA1S | p.Arg1239 |
| intermittent porphyria | HMBS | R173W |
| isolated agammaglobulinemia | E47 | E555K |
| Lattice corneal dystrophy type I | TGFBI | Arg124Cys |
| LCHAD deficiency | HADHA | Glu474Gln |
| Leber congenital amaurosis 2 | RPE65 | R44* |
| Leber congenital amaurosis 2 | RPE65 | IVS1 |
| Leber congenital amaurosis 2 | RPE65 | G − A, +5 |
| Lesch-Nyhan syndrome | HPRTI | R51* |
| Lesch-Nyhan syndrome | HPRTI | R170* |
| Limb-girdle muscular dystrophy, type 2D | SGCA | Arg77Cys |
| Marteauz- Lamy Syndrome (MSPVI) | ARSB | Y210C |
| Mediterranean G6PD deficiency | G6PD | S188D |
| medium-chain acyl-CoA dehydrogenase | ACADM | K329E |
| deficiency | | |
| medium-chain acyl-CoA dehydrogenase | ACADM | K329E |
| deficiency | | |
| medium-chain acyl-CoA dehydrogenase | ACADM | K329E |
| deficiency | | |
| Meesmann epithelial corneal dystrophy | KRT12 | L132P |
| metachromatic leukodystrophy | ARSA | P426L |
| metachromatic leukodystrophy | ARSA | c.459 + 1G > A |
| Morquio Syndrome (MPSIVA) | GALNS | R386C |
| Mucolipidosis IV | MCOLN1 | 406 − 2A > G |
| Mucolipidosis IV | MCOLN1 | 511 6943del |
| Neimann-Pick disease type A | SMPDI | L302P |
| Neuronal ceroid lipofuscinosis (NCL) | CLN2 | R208* |
| neuronal ceroid lipofuscinosis 1 | PPT1 | R151* |
| Parkinson's disease | LRRK2 | G2019S |
| Pendred syndrome | PDS | T461P |
| Pendred syndrome | PDS | L236P |
| Pendred syndrome | PDS | c.1001 + 1G > A |
| Pendred syndrome | PDS | IVS8, +1 G > A, |
| phenylketonuria | PAH | R408W |
| phenylketonuria | PAH | I65T |
| phenylketonuria | PAH | R261Q |
| phenylketonuria | PAH | IVS10 − 11G > A |
| phenylketonuria | PCDH15 | R245* |
| phenylketonuria | PCDH15 | R245* |
| Pompe disease | GAA | c.−32 − 13T > G |
| Primary ciliary dyskinesia | DNAI1 | IVS1 + 2_3insT |
| Primary ciliary dyskinesia | DNAH5 | 10815delT |
| primary hypoxaluria | AGXT | G170R |
| Progressive familial intrahepatic cholestasis type | ABCB11 | D482G (c.1445A > G) |
| 2 | | |
| Progressive familial intrahepatic cholestasis type | ABCB11 | E297G |
| 2 | | |
| Propionic acidemia | PCCB; PCCA | c.1218_1231del14ins12 |
| pseudoxanthoma elasticum | ABCC6 | R1141* |
| Pyruvate kinase deficiency (PKD) | PKLR | c.1456c −> T |
| retinitis pigmentos | USH2a | C759F |
| retinitis pigmentosa | IMPDHI | D226N |
| retinitis pigmentosa | PDE6A | V685M |
| retinitis pigmentosa | PDE6A | D670G |
| retinitis pigmentosa | PRPF3 | T494M |
| retinitis pigmentosa | PRPF8 | H2309R |
| retinitis pigmentosa | RHO | P23H |
| retinitis pigmentosa | RHO | P347L |
| retinitis pigmentosa | RHO | P347L |
| retinitis pigmentosa | RHO | D190N |
| retinitis pigmentosa | RPI | R667* |
| retinitis pigmentosa/Usher syndrome type 1C | USH1C | V72V |
| Rett syndrome | MECP2 | R106W |
| Rett syndrome | MECP2 | R133C |
| Rett syndrome | MECP2 | R306C |
| Rett syndrome | MECP2 | R168* |
| Rett syndrome | MECP2 | R255* |
| Sanfilippo syndrome A (MPSIIIA) | SGSH | R74C |
| Sanfilippo syndrome A (MPSIIIA) | SGSH | R245H |
| Sanfilippo syndrome B (MPSIIIB) | NAGLU | R297* |
| Sanfilippo syndrome B (MPSIIIB) | NAGLU | Y140C |
| severe combined immunodeficiency | ADA | G216R |
| severe combined immunodeficiency | ADA | G216R |
| severe combined immunodeficiency | ADA | Q3* |
| sickle cell disease | HBB | E6V |
| sickle cell disease | HBB | E6V |
| sickle cell disease | HBB | E6V |
| sickle cell disease | HBB | E26K |
| sickle cell disease | HBB | E26K |
| sickle cell disease | HBB | E7K |
| sickle cell disease | HBB | c.−138C > T |
| sickle cell disease | HBB | IVS2 |
| sickle cell disease | HBB | 654 C > T |
| Sly Syndrome (MPSVII) | GUSB | L175F |
| Stargardt disease | ABCA4 | A1038V |
| Stargardt disease | ABCA4 | A1038V |
| Stargardt disease | ABCA4 | L541P |
| Stargardt disease | ABCA4 | G1961E |
| Stargardt disease | ABCA4 | G1961E |
| Stargardt disease | ABCA4 | G1961E |
| Stargardt disease | ABCA4 | G1961E |
| Stargardt disease | ABCA4 | c.2588G > C |
| Stargardt disease | ABCA4 | c.5461 − 10 T > C |
| Stargardt disease | ABCA4 | c.5714 + 5G > A |
| Tay Sachs | HEXA | InsTATC1278 |
| tyrosinemia type 1 | FAH | P261L |
| Usher syndrome type 1F | PCDH15 | R245* |
| variegate porphyria | PPOX | R59W |
| VCP myopathy (IBMPFD) 1 | VCP | R1555X |
| von Gierke disease | G6PC | Q347* |
| von Gierke disease | G6PC | Q347* |
| von Gierke disease | G6PC | Q347* |
| von Gierke disease | G6PC | R83C |
| Wilson's Disease | ATP7B | E297G |
| X-linked myotubular myopathy | MTMI | c.1261 − 10A > G |
| X-linked retinoschisis | RS1 | R102W |
| X-linked retinoschisis | RS1 | R141C |

^{#}See J T den Dunnen and S E Antonarakis, Hum Mutat. 2000; 15(1): 7-12, herein incorporated by reference in its entirety, for details of the nomenclatures of gene mutations.
*means a stop codon.

### Compensatory Edits

In some embodiments, the systems or methods provided herein can be used to introduce a compensatory edit. In some embodiments, the compensatory edit is at a position of a gene associated with a disease or disorder, which is different from the position of a disease-causing mutation. In some embodiments, the compensatory mutation is not in the gene containing the causitive mutation. In some embodiments, the compensatory edit can negate or compensate for a disease-causing mutation. In some embodiments, the compensatory edit can be introduced by the systems or methods provided herein to suppress or reverse the mutant effect of a disease-causing mutation.

Table 24 provides exemplary indications (column 1), genes (column 2), and compensatory edits that can be introduced using the systems or methods described herein (column 3). In some embodiments, the compensatory edits provided in Table 24 can be introduced to suppress or reverse the mutant effect of a disease-causing mutation.

TABLE 24
Indications, genes, compensatory edits,
and exemplary design features.

| Disease | Gene | Nucleotide Change^{#} |
|---|---|---|
| Alpha-1 Antitrypsin Deficiency | SERPINAI | F51L |
| Alpha-1 Antitrypsin Deficiency | SERPINAI | M374I |
| Alpha-1 Antitrypsin Deficiency | SERPINAI | A348V/A347V |
| Alpha-1 Antitrypsin Deficiency | SERPINAI | K387R |
| Alpha-1 Antitrypsin Deficiency | SERPINAI | T59A |
| Alpha-1 Antitrypsin Deficiency | SERPINAI | T68A |
| ATTR amyloidosis | TTR | Al08V |
| ATTR amyloidosis | TTR | Rl04H |
| ATTR amyloidosis | TTR | T119M |
| Cystic fibrosis | CFTR | R555K |
| Cystic fibrosis | CFTR | F409L |
| Cystic fibrosis | CFTR | F433L |
| Cystic fibrosis | CFTR | H667R |
| Cystic fibrosis | CFTR | Rl070W |
| Cystic fibrosis | CFTR | R29K |
| Cystic fibrosis | CFTR | R553Q |
| Cystic fibrosis | CFTR | 1539T |
| Cystic fibrosis | CFTR | G550E |
| Cystic fibrosis | CFTR | F429S |
| Cystic fibrosis | CFTR | Q637R |
| Sickle cell disease | HBB | A70T |
| Sickle cell disease | HBB | A70V |
| Sickle cell disease | HBB | L88P |
| Sickle cell disease | HBB | F85L and/or F85P |
| Sickle cell disease | HBB | E22G |
| Sickle cell disease | HBB | G16D and/or G16N |

^{#}See J T den Dunnen and S E Antonarakis, Hum Mutat. 2000; 15(1): 7-12, herein incorporated by reference in its entirety, for details of the nomenclatures of gene mutations.

### Regulatory Edits

In some embodiments, the systems or methods provided herein can be used to introduce a regulatory edit. In some embodiments, the regulatory edit is introduced to a regulatory sequence of a gene, for example, a gene promoter, gene enhancer, gene repressor, or a sequence that regulates gene splicing. In some embodiments, the regulatory edit increases or decreases the expression level of a target gene. In some embodiments, the target gene is the same as the gene containing a disease-causing mutation. In some embodiment, the target gene is different from the gene containing a disease-causing mutation. For example, the systems or methods provided herein can be used to upregulate the expression of fetal hemoglobin by introducing a regulatory edit at the promoter of bcl11a, thereby treating sickle cell disease.

Table 25 provides exemplary indications (column 1), genes (column 2), and regulatory edits that can be introduced using the systems or methods described herein (column 3).

TABLE 25
Indications, genes, and compensatory regulatory edits.

| Disease | Gene | Nucleotide Change^{#} |
|---|---|---|
| homozygous familial | LDLR | c.81C > T |
| hypercholesterolaemia | | |
| Porphyrias | ALAS1 | c.3G > A |
| Porphyrias | ALAS1 | c.2T > C |
| Porphyrias | ALAS1 | c.46C > T |
| Porphyrias | ALAS1 | c.91C > T |
| Porphyrias | ALAS1 | c.91C > T |
| Porphyrias | ALAS1 | c.226C > T |
| Porphyrias | ALAS1 | c.226C > T |
| Porphyrias | ALAS1 | c.226C > T |
| Porphyrias | ALAS1 | c.229C > T |
| Porphyrias | ALAS1 | c.247C > T |
| Porphyrias | ALAS1 | c.247C > T |
| Porphyrias | ALAS1 | c.250C > T |
| Porphyrias | ALAS1 | c.250C > T |
| Porphyrias | ALAS1 | c.340C > T |
| Porphyrias | ALAS1 | c.340C > T |
| Porphyrias | ALAS1 | c.349C > T |
| Porphyrias | ALAS1 | c.391C > T |
| Porphyrias | ALAS1 | c.391C > T |
| Porphyrias | ALAS1 | c.403C > T |
| Porphyrias | ALAS1 | c.403C > T |
| Porphyrias | ALAS1 | c.199 + 1G > A |
| Porphyrias | ALAS1 | c.199 + 1G > A |
| Porphyrias | ALAS1 | c.199 + 1G > A |
| Porphyrias | ALAS1 | c.199 + 1G > A |
| Porphyrias | ALAS1 | c.199 + 2T > C |
| Porphyrias | ALAS1 | c.199 + 2T > C |
| Porphyrias | ALAS1 | c.199 + 2T > C |
| Porphyrias | ALAS1 | c.199 + 2T > C |
| Porphyrias | ALAS1 | c.200 − 2A > G |
| Porphyrias | ALAS1 | c.427 + 1G > A |
| Porphyrias | ALAS1 | c.427 + 2T > C |
| Porphyrias | ALAS1 | c.1165 + 1G > A |
| Porphyrias | ALAS1 | c.1 165 + 2T > C |
| Porphyrias | ALAS1 | c.1166 − 1A > G |
| Porphyrias | ALAS1 | c.133 1 − 2A > G |
| sickle cell disease | BCL11A | c.386 − 24278G > A |
| sickle cell disease | BCL11A | c.386 − 24983T > C |
| sickle cell disease | HBG1 | c.−167C > T |
| sickle cell disease | HBG1 | c.−170G > A |
| sickle cell disease | HBG1 | c.−249C > T |
| sickle cell disease | HBG2 | c.−211C > T |
| sickle cell disease | HBG2 | c.−228T > C |
| sickle cell disease | HBG1/2 | C. −198 T > C |
| sickle cell disease | HBG1/2 | C. −198 T > C |
| sickle cell disease | HBG1/2 | C. −198 T > C |
| sickle cell disease | HBG1/2 | C. −198 T > C |
| sickle cell disease | HBG1/2 | C. −198 T > C |
| sickle cell disease | HBG1/2 | C. −198 T > C |
| sickle cell disease | HBG1/2 | C. −198 T > C |
| sickle cell disease | HBG1/2 | C. −175 T > C |
| sickle cell disease | HBG1/2 | C. −175 T > C |
| sickle cell disease | HBG1/2 | C. −175 T > C |
| sickle cell disease | HBG1/2 | C. −175 T > C |
| sickle cell disease | HBG1/2 | C. −175 T > C |
| sickle cell disease | HBG1/2 | C. −114~−102 deletion |
| sickle cell disease | HBG1/2 | C. −114~−102 deletion |
| sickle cell disease | HBG1/2 | C. −114~−102 deletion |
| sickle cell disease | HBG1/2 | C. −114~−102 deletion |
| sickle cell disease | HBG1/2 | C. −114~−102 deletion |
| sickle cell disease | HBG1/2 | C. −114~−102 deletion |
| sickle cell disease | HBG1/2 | C. −114~−102 deletion |
| sickle cell disease | HBG1/2 | C. −114~−102 deletion |
| sickle cell disease | HBG1/2 | C. −114~−102 deletion |
| sickle cell disease | HBG1/2 | C. −114~−102 deletion |
| sickle cell disease | HBG1/2 | C. −114~−102 deletion |
| sickle cell disease | HBG1/2 | c. −90 BCLllA Binding |
| sickle cell disease | HBG1/2 | c. −90 BCLllA Binding |
| sickle cell disease | HBG1/2 | C. −202 C > T, −201 |
| | | C > T, −198 T > C, −197 |
| | | C > T, −196 C > T, −195 |
| | | C > G |
| sickle cell disease | HBG1/2 | C. −197 C > T, −196 |
| | | C > T, −195 C > G |

^{#}See J T den Dunnen and S E Antonarakis, Hum Mutat. 2000; 15(1): 7-12, herein incorporated by reference in its entirety, for details of the nomenclatures of gene mutations.

### Repeat Expansion Diseases

In some embodiments, the systems or methods provided herein can be used to a repeat expansion disease, for example, a repeat expansion disease provided in Table 26. Table 26 provides the indication (column 1), the gene (column 2), minimal repeat sequence of the repeat that is expanded in the condition (column 3), and the location of the repeat relative to the listed gene for each indication (column 4). In some embodiments, the systems, or methods provided herein, for example, those comprising gene modifying polypeptides, can be used to treat repeat expansion diseases by resetting the number of repeats at the locus according to a customized RNA template.

TABLE 26
Exemplary repeat expansion diseases, genes, causal repeats, and repeat locations.

| Disease | Gene | Causal repeat | Repeat location |
|---|---|---|---|
| myotonic dystrophy 1 | DMPK/DM1 | CTG | 3′ UTR |
| myotonic dystrophy 2 | ZNF9/CNBP | CCTG | Intron 1 |
| dentatorubral-pallidoluysian atrophy | ATN1 | CAG | Coding |
| fragile X mental retardation | FMR1 | CGG | 5′ UTR |
| syndrome | | | |
| fragile X E mental retardation | FMR2 | GCC | 5′ UTR |
| Friedreich′s ataxia | FXN | GAA | Intron |
| fragile X tremor ataxia syndrome | FMR1 | CGG | 5′ UTR |
| Huntington′s disease | HTT | CAG | Coding |
| Huntington′s disease-like 2 | JPH3 | CTG | 3′ UTR, coding |
| myoclonic epilepsy of Unverricht | CSTB | CCCCGCCCCGCG | Promoter |
| and Lundborg | | (SEQ ID NO: | |
| | | 15434) | |
| oculopharyngeal muscular dystrophy | PABPN1 | GCG | Coding |
| spinal and bulbar muscular atrophy | AR | CAG | Coding |
| spinocerebellar ataxia 1 | ATXN1 | CAG | Coding |
| spinocerebellar ataxia 2 | ATXN2 | CAG | Coding |
| spinocerebellar ataxia 3 | ATXN3 | CAG | Coding |
| spinocerebellar ataxia 6 | CACNA1A | CAG | Coding |
| spinocerebellar ataxia 7 | ATXN7 | CAG | Coding |
| spinocerebellar ataxia 8 | ATXN8 | CTG/CAG | CTG/CAG |
| | | | (ATXN8) |
| spinocerebellar ataxia 10 | ATXN10 | ATTCT | Intron |
| spinocerebellar ataxia 12 | PPP2R2B | CAG | Promoter, 5′ |
| | | | UTR? |
| spinocerebellar ataxia 17 | TBP | CAG | Coding |
| Syndromic/non-syndromic X-linked | ARX | GCG | Coding |
| mental retardation | | | |

### Exemplary Heterologous Object Sequences

In some embodiments, the systems or methods provided herein comprise a heterologous object sequence, wherein the heterologous object sequence or a reverse complementary sequence thereof, encodes a protein (e.g., an antibody) or peptide. In some embodiments, the therapy is one approved by a regulatory agency such as FDA.

In some embodiments, the protein or peptide is a protein or peptide from the THPdb database (Usmani et al. PLoS One 12(7):e0181748 (2017), herein incorporated by reference in its entirety. In some embodiments, the protein or peptide is a protein or peptide disclosed in Table 28. In some embodiments, the systems, or methods disclosed herein, for example, those comprising gene modifying polypeptides, may be used to integrate an expression cassette for a protein or peptide from Table 28 into a host cell to enable the expression of the protein or peptide in the host. In some embodiments, the sequences of the protein or peptide in the first column of Table 28 can be found in the patents or applications provided in the third column of Table 28, incorporated by reference in their entireties.

In some embodiments, the protein or peptide is an antibody disclosed in Table 1 of Lu et al. J Biomed Sci 27(1):1 (2020), herein incorporated by reference in its entirety. In some embodiments, the protein or peptide is an antibody disclosed in Table 29. In some embodiments, the systems, or methods disclosed herein, for example, those comprising gene modifying polypeptides, may be used to integrate an expression cassette for an antibody from Table 29 into a host cell to enable the expression of the antibody in the host. In some embodiments, a system or method described herein is used to express an agent that binds a target of column 2 of Table 29 (e.g., a monoclonal antibody of column 1 of Table 29) in a subject having an indication of column 3 of Table 29.

TABLE 28
Exemplary protein and peptide therapeutics.

| Therapeutic peptide | Category | Patent Number |
|---|---|---|
| Lepirudin | Antithrombins and Fibrinolytic | CA1339104 |
| | Agents | |
| Cetuximab | Antineoplastic Agents | CA1340417 |
| Dor se alpha | Enzymes | CA2184581 |
| Denileukin diftitox | Antineoplastic Agents | |
| Etanercept | Immunosuppressive Agents | CA2476934 |
| Bivalirudin | Antithrombins | U.S. Pat. No. 7,582,727 |
| Leuprolide | Antineoplastic Agents | |
| Peginterferon alpha-2a | Immunosuppressive Agents | CA2203480 |
| Alteplase | Thrombolytic Agents | |
| Interferon alpha-n1 | Antiviral Agents | |
| Darbepoetin alpha | Anti-anemic Agents | CA2165694 |
| Reteplase | Fibrinolytic Agents | CA2107476 |
| Epoetin alpha | Hematinics | CA1339047 |
| Salmon Calcitonin | Bone Density Conservation | U.S. Pat. No. 6,440,392 |
| | Agents | |
| Interferon alpha-n3 | Immunosuppressive Agents | |
| Pegfilgrastim | Immunosuppressive Agents | CA1341537 |
| Sargramostim | Immunosuppressive Agents | CA1341150 |
| Secretin | Diagnostic Agents | |
| Peginterferon alpha-2b | Immunosuppressive Agents | CA1341567 |
| Asparagi se | Antineoplastic Agents | |
| Thyrotropin alpha | Diagnostic Agents | U.S. Pat. No. 5,840,566 |
| Antihemophilic Factor | Coagulants and Thrombotic agents | CA2124690 |
| A kinra | Antirheumatic Agents | CA2141953 |
| Gramicidin D | Anti-Bacterial Agents | |
| Intravenous | Immunologic Factors | |
| Immunoglobulin | | |
| Anistreplase | Fibrinolytic Agents | |
| Insulin Regular | Antidiabetic Agents | |
| Tenecteplase | Fibrinolytic Agents | CA2129660 |
| Menotropins | Fertility Agents | |
| Interferon gamma-1b | Immunosuppressive Agents | U.S. Pat. No. 6,936,695 |
| Interferon alpha-2a, | | CA2172664 |
| Recombi nt | | |
| Coagulation factor VIIa | Coagulants | |
| Oprelvekin | Antineoplastic Agents | |
| Palifermin | Anti-Mucositis Agents | |
| Glucagon recombi nt | Hypoglycemic Agents | |
| Aldesleukin | Antineoplastic Agents | |
| Botulinum Toxin Type B | Antidystonic Agents | |
| Omalizumab | Anti-Allergic Agents | CA2113813 |
| Lutropin alpha | Fertility Agents | U.S. Pat. No. 5,767,251 |
| Insulin Lispro | Hypoglycemic Agents | U.S. Pat. No. 5,474,978 |
| Insulin Glargine | Hypoglycemic Agents | U.S. Pat. No. 7,476,652 |
| Collage se | | |
| Rasburicase | Gout Suppressants | CA2175971 |
| Adalimumab | Antirheumatic Agents | CA2243459 |
| Imiglucerase | Enzyme Replacement Agents | U.S. Pat. No. 5,549,892 |
| Abciximab | Anticoagulants | CA1341357 |
| Alpha-1-protei se inhibitor | Serine Protei se Inhibitors | |
| Pegaspargase | Antineoplastic Agents | |
| Interferon beta-1a | Antineoplastic Agents | CA1341604 |
| Pegademase bovine | Enzyme Replacement Agents | |
| Human Serum Albumin | Serum substitutes | U.S. Pat. No. 6,723,303 |
| Eptifibatide | Platelet Aggregation Inhibitors | U.S. Pat. No. 6,706,681 |
| Serum albumin iodo ted | Diagnostic Agents | |
| Infliximab | Antirheumatic Agents, Anti- | CA2106299 |
| | Inflammatory Agents, Non- | |
| | Steroidal, Dermatologic Agents, | |
| | Gastrointesti 1 Agents and | |
| | Immunosuppressive Agents | |
| Follitropin beta | Fertility Agents | U.S. Pat. No. 7,741,268 |
| Vasopressin | Antidiuretic Agents | |
| Interferon beta-1b | Adjuvants, Immunologic and | CA1340861 |
| | Immunosuppressive Agents | |
| Interferon alphacon-1 | Antiviral Agents and | CA1341567 |
| | Immunosuppressive Agents | |
| Hyaluronidase | Adjuvants, Anesthesia and | |
| | Permeabilizing Agents | |
| Insulin, porcine | Hypoglycemic Agents | |
| Trastuzumab | Antineoplastic Agents | CA2103059 |
| Rituximab | Antineoplastic Agents, | CA2149329 |
| | Immunologic Factors and | |
| | Antirheumatic Agents | |
| Basiliximab | Immunosuppressive Agents | CA2038279 |
| Muromo b | Immunologic Factors and | |
| | Immunosuppressive Agents | |
| Digoxin Immune Fab | Antidotes | |
| (Ovine) | | |
| Ibritumomab | | CA2149329 |
| Daptomycin | | U.S. Pat. No. 6,468,967 |
| Tositumomab | | |
| Pegvisomant | Hormone Replacement Agents | U.S. Pat. No. 5,849,535 |
| Botulinum Toxin Type A | Neuromuscular Blocking Agents, | CA2280565 |
| | Anti-Wrinkle Agents and | |
| | Antidystonic Agents | |
| Pancrelipase | Gastrointesti 1 Agents and Enzyme | |
| | Replacement Agents | |
| Streptoki se | Fibrinolytic Agents and | |
| | Thrombolytic Agents | |
| Alemtuzumab | | CA1339198 |
| Alglucerase | Enzyme Replacement Agents | |
| Capromab | Indicators, Reagents and | |
| | Diagnostic Agents | |
| Laronidase | Enzyme Replacement Agents | |
| Urofollitropin | Fertility Agents | U.S. Pat. No. 5,767,067 |
| Efalizumab | Immunosuppressive Agents | |
| Serum albumin | Serum substitutes | U.S. Pat. No. 6,723,303 |
| Choriogo dotropin alpha | Fertility Agents and Go dotropins | U.S. Pat. No. 6,706,681 |
| Antithymocyte globulin | Immunologic Factors and | |
| | Immunosuppressive Agents | |
| Filgrastim | Immunosuppressive Agents, | CA1341537 |
| | Antineutropenic Agents and | |
| | Hematopoietic Agents | |
| Coagulation factor ix | Coagulants and Thrombotic | |
| | Agents | |
| Becaplermin | Angiogenesis Inducing Agents | CA1340846 |
| Agalsidase beta | Enzyme Replacement Agents | CA2265464 |
| Interferon alpha-2b | Immunosuppressive Agents | CA1341567 |
| Oxytocin | Oxytocics, Anti-tocolytic Agents | |
| | and Labor Induction Agents | |
| Enfuvirtide | HIV Fusion Inhibitors | U.S. Pat. No. 6,475,491 |
| Palivizumab | Antiviral Agents | CA2197684 |
| Daclizumab | Immunosuppressive Agents | |
| Bevacizumab | Angiogenesis Inhibitors | CA2286330 |
| Arcitumomab | Diagnostic Agents | U.S. Pat. No. 8,420,081 |
| Arcitumomab | Diagnostic Agents | U.S. Pat. No. 7,790,142 |
| Eculizumab | | CA2189015 |
| Panitumumab | | |
| Ranibizumab | Ophthalmics | CA2286330 |
| Idursulfase | Enzyme Replacement Agents | |
| Alglucosidase alpha | Enzyme Replacement Agents | CA2416492 |
| Exe tide | Hypoglycemic Agents | U.S. Pat. No. 6,872,700 |
| Mecasermin | | U.S. Pat. No. 5,681,814 |
| Pramlintide | | U.S. Pat. No. 5,686,411 |
| Galsulfase | Enzyme Replacement Agents | |
| Abatacept | Antirheumatic Agents and | CA2110518 |
| | Immunosuppressive Agents | |
| Cosyntropin | Hormones and Diagnostic Agents | |
| Corticotropin | | |
| Insulin aspart | Hypoglycemic Agents and | U.S. Pat. No. 5,866,538 |
| | Antidiabetic Agents | |
| Insulin detemir | Antidiabetic Agents | U.S. Pat. No. 5,750,497 |
| Insulin glulisine | Antidiabetic Agents | U.S. Pat. No. 6,960,561 |
| Pegaptanib | Intended for the prevention of | |
| | respiratory distress syndrome | |
| | (RDS) in premature infants at high | |
| | risk for RDS. | |
| Nesiritide | | |
| Thymalphasin | | |
| Defibrotide | Antithrombins | |
| tural alpha interferon OR | | |
| multiferon | | |
| Glatiramer acetate | | |
| Preotact | | |
| Teicoplanin | Anti-Bacterial Agents | |
| Ca kinumab | Anti-Inflammatory Agents and | |
| | Monoclo 1 antibodies | |
| Ipilimumab | Antineoplastic Agents and | CA2381770 |
| | Monoclo 1 antibodies | |
| Sulodexide | Antithrombins and Fibrinolytic | |
| | Agents and Hypoglycemic Agents | |
| | and Anticoagulants and | |
| | Hypolipidemic Agents | |
| Tocilizumab | | CA2201781 |
| Teriparatide | Bone Density Conservation | U.S. Pat. No. 6,977,077 |
| | Agents | |
| Pertuzumab | Monoclo 1 antibodies | CA2376596 |
| Rilo cept | Immunosuppressive Agents | U.S. Pat. No. 5,844,099 |
| Denosumab | Bone Density Conservation | CA2257247 |
| | Agents and Monoclo 1 antibodies | |
| Liraglutide | | U.S. Pat. No. 6,268,343 |
| Golimumab | Antipsoriatic Agents and Monoclo | |
| | 1 antibodies and TNF inhibitor | |
| Belatacept | Antirheumatic Agents and | |
| | Immunosuppressive Agents | |
| Buserelin | | |
| Velaglucerase alpha | Enzymes | U.S. Pat. No. 7,138,262 |
| Tesamorelin | | U.S. Pat. No. 5,861,379 |
| Brentuximab vedotin | | |
| Taliglucerase alpha | Enzymes | |
| Belimumab | Monoclo 1 antibodies | |
| Aflibercept | Antineoplastic Agents and | U.S. Pat. No. 7,306,799 |
| | Ophthalmics | |
| Asparagi se erwinia | Enzymes | |
| chrysanthemi | | |
| Ocriplasmin | Ophthalmics | |
| Glucarpidase | Enzymes | |
| Teduglutide | | U.S. Pat. No. 5,789,379 |
| Raxibacumab | Anti-Infective Agents and | |
| | Monoclo 1 antibodies | |
| Certolizumab pegol | TNF inhibitor | CA2380298 |
| Insulin, isophane | Hypoglycemic Agents and | |
| | Antidiabetic Agents | |
| Epoetin zeta | | |
| Obinutuzumab | Antineoplastic Agents | |
| Fibrinolysin aka plasmin | | U.S. Pat. No. 3,234,106 |
| Follitropin alpha | | |
| Romiplostim | Colony-Stimulating Factors and | |
| | Thrombopoietic Agents | |
| Luci ctant | Pulmo ry surfactants | U.S. Pat. No. 5,407,914 |
| talizumab | Immunosuppressive agents | |
| Aliskiren | Renin inhibitor | |
| Ragweed Pollen Extract | | |
| Secukinumab | Inhibitor | US20130202610 |
| Somatotropin Recombi nt | Hormone Replacement Agents | CA1326439 |
| Drotrecogin alpha | Antisepsis | CA2036894 |
| Alefacept | Dermatologic and | |
| | Immunosupressive agents | |
| OspA lipoprotein | Vaccines | |
| Uroki se | | U.S. Pat. No. 4,258,030 |
| Abarelix | Anti-Testosterone Agents | U.S. Pat. No. 5,968,895 |
| Sermorelin | Hormone Replacement Agents | |
| Aprotinin | | U.S. Pat. No. 5,198,534 |
| Gemtuzumab ozogamicin | Antineoplastic agents and | U.S. Pat. No. 5,585,089 |
| | Immunotoxins | |
| Satumomab Pendetide | Diagnostic Agents | |
| Albiglutide | Drugs used in diabetes; alimentary | |
| | tract and metabolism; blood | |
| | glucose lowering drugs, excl. | |
| | insulins. | |
| Alirocumab | | |
| Ancestim | | |
| Antithrombin alpha | | |
| Antithrombin III human | | |
| Asfotase alpha | Enzymes Alimentary Tract and | |
| | Metabolism | |
| Atezolizumab | | |
| Autologous cultured | | |
| chondrocytes | | |
| Beractant | | |
| Bli tumomab | Antineoplastic Agents | US20120328618 |
| | Immunosuppressive Agents | |
| | Monoclo 1 antibodies | |
| | Antineoplastic and | |
| | Immunomodulating Agents | |
| C1 Esterase Inhibitor | | |
| (Human) | | |
| Coagulation Factor XIII A- | | |
| Subunit (Recombi nt) | | |
| Conestat alpha | | |
| Daratumumab | Antineoplastic Agents | |
| Desirudin | | |
| Dulaglutide | Hypoglycemic Agents; Drugs | |
| | Used in Diabetes; Alimentary | |
| | Tract and Metabolism; Blood | |
| | Glucose Lowering Drugs, Excl. | |
| | Insulins | |
| Elosulfase alpha | Enzymes; Alimentary Tract and | |
| | Metabolism | |
| Elotuzumab | | US2014055370 |
| Evolocumab | Lipid Modifying Agents, Plain; | |
| | Cardiovascular System | |
| Fibrinogen Concentrate | | |
| (Human) | | |
| Filgrastim-sndz | | |
| Gastric intrinsic factor | | |
| Hepatitis B immune | | |
| globulin | | |
| Human calcitonin | | |
| Human clostridium tetani | | |
| toxoid immune globulin | | |
| Human rabies virus | | |
| immune globulin | | |
| Human Rho(D) immune | | |
| globulin | | |
| Hyaluronidase (Human | | U.S. Pat. No. 7,767,429 |
| Recombi nt) | | |
| Idarucizumab | Anticoagulant | |
| Immune Globulin Human | Immunologic Factors; | |
| | Immunosuppressive Agents; Anti- | |
| | Infective Agents | |
| Vedolizumab | Immunosupressive agent, | US2012151248 |
| | Antineoplastic agent | |
| Ustekinumab | Deramtologic agent, | |
| | Immunosuppressive agent, | |
| | antineoplastic agent | |
| Turoctocog alpha | | |
| Tuberculin Purified Protein | | |
| Derivative | | |
| Simoctocog alpha | Antihaemorrhagics: blood | |
| | coagulation factor VIII | |
| Siltuximab | Antineoplastic and | U.S. Pat. No. 7,612,182 |
| | Immunomodulating Agents, | |
| | Immunosuppressive Agents | |
| Sebelipase alpha | Enzymes | |
| Sacrosidase | Enzymes | |
| Ramucirumab | Antineoplastic and | US2013067098 |
| | Immunomodulating Agents | |
| Prothrombin complex | | |
| concentrate | | |
| Poractant alpha | Pulmo ry Surfactants | |
| Pembrolizumab | Antineoplastic and | US2012135408 |
| | Immunomodulating Agents | |
| Peginterferon beta-1a | | |
| Ofatumumab | Antineoplastic and | U.S. Pat. No. 8,337,847 |
| | Immunomodulating Agents | |
| Obiltoxaximab | | |
| Nivolumab | Antineoplastic and | US2013173223 |
| | Immunomodulating Agents | |
| Necitumumab | | |
| Metreleptin | | US20070099836 |
| Methoxy polyethylene | | |
| glycol-epoetin beta | | |
| Mepolizumab | Antineoplastic and | US2008134721 |
| | Immunomodulating Agents, | |
| | Immunosuppressive Agents, | |
| | Interleukin Inhibitors | |
| Ixekizumab | | |
| Insulin Pork | Hypoglycemic Agents, | |
| | Antidiabetic Agents | |
| Insulin Degludec | | |
| Insulin Beef | | |
| Thyroglobulin | Hormone therapy | U.S. Pat. No. 5,099,001 |
| Anthrax immune globulin | Plasma derivative | |
| human | | |
| Anti-inhibitor coagulant | Blood Coagulation Factors, | |
| complex | Antihemophilic Agent | |
| Anti-thymocyte Globulin | Antibody | |
| (Equine) | | |
| Anti-thymocyte Globulin | Antibody | |
| (Rabbit) | | |
| Brodalumab | Antineoplastic and | |
| | Immunomodulating Agents | |
| C1 Esterase Inhibitor | Blood and Blood Forming Organs | |
| (Recombi nt) | | |
| Ca kinumab | Antineoplastic and | |
| | Immunomodulating Agents | |
| Chorionic Go dotropin | Hormones | U.S. Pat. No. 6,706,681 |
| (Human) | | |
| Chorionic Go dotropin | Hormones | U.S. Pat. No. 5,767,251 |
| (Recombi nt) | | |
| Coagulation factor X | Blood Coagulation Factors | |
| human | | |
| Dinutuximab | Antibody, Immunosuppresive | US20140170155 |
| | agent, Antineoplastic agent | |
| Efmoroctocog alpha | Antihemophilic Factor | |
| Factor IX Complex | Antihemophilic agent | |
| (Human) | | |
| Hepatitis A Vaccine | Vaccine | |
| Human Varicella-Zoster | Antibody | |
| Immune Globulin | | |
| Ibritumomab tiuxetan | Antibody, Immunosuppressive | CA2149329 |
| | Agents | |
| Lenograstim | Antineoplastic and | |
| | Immunomodulating Agents | |
| Pegloticase | Enzymes | |
| Protamine sulfate | Heparin Antagonists, Hematologic | |
| | Agents | |
| Protein S human | Anticoagulant plasma protein | |
| Sipuleucel-T | Antineoplastic and | U.S. Pat. No. 8,153,120 |
| | Immunomodulating Agents | |
| Somatropin recombi nt | Hormones, Hormone Substitutes, | CA1326439, CA2252535, |
| | and Hormone Antagonists | U.S. Pat. No. 5,288,703, |
| | | U.S. Pat. No. 5,849,700, |
| | | U.S. Pat. No. 5,849,704, |
| | | U.S. Pat. No. 5,898,030 |
| | | U.S. Pat. No. 6,004,297, |
| | | U.S. Pat. No. 6,152,897, |
| | | U.S. Pat. No. 6,235,004, |
| | | U.S. Pat. No. 6,899,699 |
| Susoctocog alpha | Blood coagulation factors, | |
| | Antihaemorrhagics | |
| Thrombomodulin alpha | Anticoagulant agent, Antiplatelet | |
| | agent | |

TABLE 29
Exemplary monoclonal antibody therapies.

| mAb | Target | Indication |
|---|---|---|
| Muromonab-CD3 | CD3 | Kidney transplant rejection |
| Abciximab | GPIIb/IIIa | Prevention of blood clots in angioplasty |
| Rituximab | CD20 | Non-Hodgkin lymphoma |
| Palivizumab | RSV | Prevention of respiratory syncytial virus |
| | | infection |
| Infliximab | TNFα | Crohn's disease |
| Trastuzumab | HER2 | Breast cancer |
| Alemtuzumab | CD52 | Chronic myeloid leukemia |
| Adalimumab | TNFα | Rheumatoid arthritis |
| Ibritumomab | CD20 | Non-Hodgkin lymphoma |
| tiuxetan | | |
| Omalizumab | IgE | Asthma |
| Cetuximab | EGFR | Colorectal cancer |
| Bevacizumab | VEGF-A | Colorectal cancer |
| Natalizumab | ITGA4 | Multiple sclerosis |
| Panitumumab | EGFR | Colorectal cancer |
| Ranibizumab | VEGF-A | Macular degeneration |
| Eculizumab | C5 | Paroxysmal nocturnal hemoglobinuria |
| Certolizumab | TNFα | Crohn's disease |
| pegol | | |
| Ustekinumab | IL-12/23 | Psoriasis |
| Canakinumab | IL-1β | Muckle-Wells syndrome |
| Golimumab | TNFα | Rheumatoid and psoriatic arthritis, ankylosing |
| | | spondylitis |
| Ofatumumab | CD20 | Chronic lymphocytic leukemia |
| Tocilizumab | IL-6R | Rheumatoid arthritis |
| Denosumab | RANKL | Bone loss |
| Belimumab | BLyS | Systemic lupus erythematosus |
| Ipilimumab | CTLA-4 | Metastatic melanoma |
| Brentuximab | CD30 | Hodgkin lymphoma, systemic anaplastic large |
| vedotin | | cell lymphoma |
| Pertuzumab | HER2 | Breast Cancer |
| Trastuzumab | HER2 | Breast cancer |
| emtansine | | |
| Raxibacumab | B. anthrasis PA | Anthrax infection |
| Obinutuzumab | CD20 | Chronic lymphocytic leukemia |
| Siltuximab | IL-6 | Castleman disease |
| Ramucirumab | VEGFR2 | Gastric cancer |
| Vedolizumab | α4β7 integrin | Ulcerative colitis, Crohn disease |
| Blinatumomab | CD19, CD3 | Acute lymphoblastic leukemia |
| Nivolumab | PD-1 | Melanoma, non-small cell lung cancer |
| Pembrolizumab | PD-1 | Melanoma |
| Idarucizumab | Dabigatran | Reversal of dabigatran-induced anticoagulation |
| Necitumumab | EGFR | Non-small cell lung cancer |
| Dinutuximab | GD2 | Neuroblastoma |
| Secukinumab | IL-17α | Psoriasis |
| Mepolizumab | IL-5 | Severe eosinophilic asthma |
| Alirocumab | PCSK9 | High cholesterol |
| Evolocumab | PCSK9 | High cholesterol |
| Daratumumab | CD38 | Multiple myeloma |
| Elotuzumab | SLAMF7 | Multiple myeloma |
| Ixekizumab | IL-17α | Psoriasis |
| Reslizumab | IL-5 | Asthma |
| Olaratumab | PDGFRα | Soft tissue sarcoma |
| Bezlotoxumab | Clostridium | Prevention of Clostridium difficile infection |
| | difficile enterotoxin B | recurrence |
| Atezolizumab | PD-L1 | Bladder cancer |
| Obiltoxaximab | B. anthrasis PA | Prevention of inhalational anthrax |
| Inotuzumab | CD22 | Acute lymphoblastic leukemia |
| ozogamicin | | |
| Brodalumab | IL-17R | Plaque psoriasis |
| Guselkumab | IL-23 p19 | Plaque psoriasis |
| Dupilumab | IL-4Rα | Atopic dermatitis |
| Sarilumab | IL-6R | Rheumatoid arthritis |
| Avelumab | PD-L1 | Merkel cell carcinoma |
| Ocrelizumab | CD20 | Multiple sclerosis |
| Emicizumab | Factor IXa, X | Hemophilia A |
| Benralizumab | IL-5Rα | Asthma |
| Gemtuzumab | CD33 | Acute myeloid leukemia |
| ozogamicin | | |
| Durvalumab | PD-L1 | Bladder cancer |
| Burosumab | FGF23 | X-linked hypophosphatemia |
| Lanadelumab | Plasma kallikrein | Hereditary angioedema attacks |
| Mogamulizumab | CCR4 | Mycosis fungoides or Sézary syndrome |
| Erenumab | CGRPR | Migraine prevention |
| Galcanezumab | CGRP | Migraine prevention |
| Tildrakizumab | IL-23 p19 | Plaque psoriasis |
| Cemiplimab | PD-1 | Cutaneous squamous cell carcinoma |
| Emapalumab | IFNγ | Primary hemophagocytic lymphohistiocytosis |
| Fremanezumab | CGRP | Migraine prevention |
| Ibalizumab | CD4 | HIV infection |
| Moxetumomab | CD22 | Hairy cell leukemia |
| pasudodox | | |
| Ravulizumab | C5 | Paroxysmal nocturnal hemoglobinuria |
| Caplacizumab | von Willebrand factor | Acquired thrombotic thrombocytopenia purpura |
| Romosozumab | Sclerostin | Osteoporosis in postmenopausal women at |
| | | increased risk of fracture |
| Risankizumab | IL-23 p19 | Plaque psoriasis |
| Polatuzumab | CD79β | Diffuse large B-cell lymphoma |
| vedotin | | |
| Brolucizumab | VEGF-A | Macular degeneration |
| Crizanlizumab | P-selectin | Sickle cell disease |

### Administration

The composition and systems described herein may be used in vitro or in vivo. In some embodiments the system or components of the system are delivered to cells (e.g., mammalian cells, e.g., human cells), e.g., in vitro or in vivo. In some embodiments, the cells are eukaryotic cells, e.g., cells of a multicellular organism, e.g., an animal, e.g., a mammal (e.g., human, swine, bovine) a bird (e.g., poultry, such as chicken, turkey, or duck), or a fish. In some embodiments, the cells are non-human animal cells (e.g., a laboratory animal, a livestock animal, or a companion animal). In some embodiments, the cell is a stem cell (e.g., a hematopoietic stem cell), a fibroblast, or a T cell. In some embodiments, the cell is a non-dividing cell, e.g., a non-dividing fibroblast or non-dividing T cell. In some embodiments, the cell is an HSC and p53 is not upregulated or is upregulated by less than 10%, 5%, 2%, or 1%, e.g., as determined according to the method described in Example 30 of PCT Application No. PCT/US2019/048607, incorporated herein by reference in its entirety. In some embodiments, the cell is a T cell, e.g., a primary T cell. In some embodiments, the cell is an induced pluripotent stem cell (iPSC). In some embodiments, the cell is a respiratory epithelial cell (e.g., a bronchial epithelial cell, e.g., a human bronchial epithelial (hBE) cell). The components of the gene modifying system may, in some instances, be delivered in the form of polypeptide, nucleic acid (e.g., DNA, RNA), and combinations thereof.

For instance, delivery can use any of the following combinations for delivering the retrotransposase (e.g., as DNA encoding the retrotransposase protein, as RNA encoding the retrotransposase protein, or as the protein itself) and the template RNA (e.g., as DNA encoding the RNA, or as RNA):
1. Retrotransposase DNA+template DNA
2. Retrotransposase RNA+template DNA
3. Retrotransposase DNA+template RNA
4. Retrotransposase RNA+template RNA
5. Retrotransposase protein+template DNA
6. Retrotransposase protein+template RNA
7. Retrotransposase virus+template virus
8. Retrotransposase virus+template DNA
9. Retrotransposase virus+template RNA
10. Retrotransposase DNA+template virus
11. Retrotransposase RNA+template virus
12. Retrotransposase protein+template virus

As indicated above, in some embodiments, the DNA or RNA that encodes the retrotransposase protein is delivered using a virus, and in some embodiments, the template RNA (or the DNA encoding the template RNA) is delivered using a virus.

In one embodiments, the system and/or components of the system are delivered as nucleic acid. For example, the gene modifying polypeptide may be delivered in the form of a DNA or RNA encoding the polypeptide and the template RNA may be delivered in the form of RNA or its complementary DNA to be transcribed into RNA. In some embodiments, the system or components of the system are delivered on 1, 2, 3, 4, or more distinct nucleic acid molecules. In some embodiments, the system or components of the system are delivered as a combination of DNA and RNA. In some embodiments, the system or components of the system are delivered as a combination of DNA and protein. In some embodiments, the system or components of the system are delivered as a combination of RNA and protein. In some embodiments, the gene modifying polypeptide is delivered as a protein.

In some embodiments, the system or components of the system are delivered to cells, e.g. mammalian cells or human cells, using a vector. The vector may be, e.g., a plasmid or a virus. In some embodiments, delivery is in vivo, in vitro, ex vivo, or in situ. In some embodiments, the virus is an adeno associated virus (AAV), a lentivirus, an adenovirus. In some embodiments, the system or components of the system are delivered to cells with a viral-like particle or a virosome. In some embodiments, the delivery uses more than one virus, viral-like particle, or virosome.

In some embodiments, nucleic acid (e.g., encoding a polypeptide, or a template DNA, or both) delivered to cells is covalently closed linear DNA, or so-called “doggybone” DNA. During its lifecycle, the bacteriophage N15 employs protelomerase to convert its genome from circular plasmid DNA to a linear plasmid DNA (Ravin et al. J Mol Biol 2001). This process has been adapted for the production of covalently closed linear DNA in vitro (see, for example, WO2010086626A1). In some embodiments, a protelomerase is contacted with a DNA containing one or more protelomerase recognition sites, wherein protelomerase results in a cut at the one or more sites and subsequent ligation of the complementary strands of DNA, resulting in the covalent linkage between the complementary strands. In some embodiments, nucleic acid (e.g., encoding a transposase, or a template DNA, or both) is first generated as circular plasmid DNA containing a single protelomerase recognition site that is then contacted with protelomerase to yield a covalently closed linear DNA. In some embodiments, nucleic acid (e.g., encoding a transposase, or a template DNA, or both) flanked by protelomerase recognition sites on plasmid or linear DNA is contacted with protelomerase to generate a covalently closed linear DNA containing only the DNA contained between the protelomerase recognition sites. In some embodiments, the approach of flanking the desired nucleic acid sequence by protelomerase recognition sites results in covalently closed circular DNA lacking plasmid elements used for bacterial cloning and maintenance. In some embodiments, the plasmid or linear DNA containing the nucleic acid and one or more protelomerase recognition sites is optionally amplified prior to the protelomerase reaction, e.g., by rolling circle amplification or PCR.

In one embodiment, the compositions and systems described herein can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral, or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Lipid nanoparticles are another example of a carrier that provides a biocompatible and biodegradable delivery system for the pharmaceutical compositions described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Exosomes can also be used as drug delivery vehicles for the compositions and systems described herein. For a review, see Ha et al. July 2016. Acta Pharmaceutica Sinica B. Volume 6, Issue 4, Pages 287-296; doi.org/10.1016/j.apsb.2016.02.001.

Fusosomes interact and fuse with target cells, and thus can be used as delivery vehicles for a variety of molecules. They generally consist of a bilayer of amphipathic lipids enclosing a lumen or cavity and a fusogen that interacts with the amphipathic lipid bilayer. The fusogen component has been shown to be engineerable in order to confer target cell specificity for the fusion and payload delivery, allowing the creation of delivery vehicles with programmable cell specificity (see, for example, the relating to fusosome design, preparation, and usage in PCT Publication No. WO/2020014209, incorporated herein by reference in its entirety).

A gene modifying system can be introduced into cells, tissues, and multicellular organisms. In some embodiments, the system or components of the system are delivered to the cells via mechanical means or physical means.

Formulation of protein therapeutics is described in Meyer (Ed.), Therapeutic Protein Drug Products: Practical Approaches to formulation in the Laboratory, Manufacturing, and the Clinic, Woodhead Publishing Series (2012).

### Tissue Specific Activity Administration

In some embodiments, a system, template RNA, or polypeptide described herein is administered to or is active in (e.g., is more active in) a target tissue, e.g., a first tissue. In some embodiments, the system, template RNA, or polypeptide is not administered to or is less active in (e.g., not active in) a non-target tissue. In some embodiments, a system, template RNA, or polypeptide described herein is useful for modifying DNA in a target tissue, e.g., a first tissue, (e.g., and not modifying DNA in a non-target tissue).

In some embodiments, a system comprises (a) a polypeptide described herein or a nucleic acid encoding the same, (b) a template nucleic acid (e.g., template RNA) described herein, and (c) one or more first tissue-specific expression-control sequences specific to the target tissue, wherein the one or more first tissue-specific expression-control sequences specific to the target tissue are in operative association with (a), (b), or (a) and (b), wherein, when associated with (a), (a) comprises a nucleic acid encoding the polypeptide.

In some embodiments, the nucleic acid in (b) comprises RNA.

In some embodiments, the nucleic acid in (b) comprises DNA.

In some embodiments, the nucleic acid in (b): (i) is single-stranded or comprises a single-stranded segment, e.g., is single-stranded DNA or comprises a single-stranded segment and one or more double stranded segments; (ii) has inverted terminal repeats; or (iii) both (i) and (ii).

In some embodiments, the nucleic acid in (b) is double-stranded or comprises a double-stranded segment.

In some embodiments, (a) comprises a nucleic acid encoding the polypeptide.

In some embodiments, the nucleic acid in (a) comprises RNA.

In some embodiments, the nucleic acid in (a) comprises DNA.

In some embodiments, the nucleic acid in (a): (i) is single-stranded or comprises a single-stranded segment, e.g., is single-stranded DNA or comprises a single-stranded segment and one or more double stranded segments; (ii) has inverted terminal repeats; or (iii) both (i) and (ii).

In some embodiments, the nucleic acid in (a) is double-stranded or comprises a double-stranded segment.

In some embodiments, the nucleic acid in (a), (b), or (a) and (b) is linear.

In some embodiments, the nucleic acid in (a), (b), or (a) and (b) is circular, e.g., a plasmid or minicircle.

In some embodiments, the heterologous object sequence is in operative association with a first promoter.

In some embodiments, the one or more first tissue-specific expression-control sequences comprises a tissue specific promoter.

In some embodiments, the tissue-specific promoter comprises a first promoter in operative association with: i. the heterologous object sequence, ii. a nucleic acid encoding the transposase, or iii. (i) and (ii).

In some embodiments, the one or more first tissue-specific expression-control sequences comprises a tissue-specific microRNA recognition sequence in operative association with: i. the heterologous object sequence, ii. a nucleic acid encoding the transposase, or iii. (i) and (ii).

In some embodiments, a system comprises a tissue-specific promoter, and the system further comprises one or more tissue-specific microRNA recognition sequences, wherein: i. the tissue specific promoter is in operative association with: I. the heterologous object sequence, II. a nucleic acid encoding the transposase, or III. (I) and (II); and/or ii. the one or more tissue-specific microRNA recognition sequences are in operative association with: I. the heterologous object sequence, II. a nucleic acid encoding the transposase, or III.(I) and (II).

In some embodiments, wherein (a) comprises a nucleic acid encoding the polypeptide, the nucleic acid comprises a promoter in operative association with the nucleic acid encoding the polypeptide.

In some embodiments, the nucleic acid encoding the polypeptide comprises one or more second tissue-specific expression-control sequences specific to the target tissue in operative association with the polypeptide coding sequence.

In some embodiments, the one or more second tissue-specific expression-control sequences comprises a tissue specific promoter.

In some embodiments, the tissue-specific promoter is the promoter in operative association with the nucleic acid encoding the polypeptide.

In some embodiments, the one or more second tissue-specific expression-control sequences comprises a tissue-specific microRNA recognition sequence.

In some embodiments, the promoter in operative association with the nucleic acid encoding the polypeptide is a tissue-specific promoter, the system further comprising one or more tissue-specific microRNA recognition sequences.

In some embodiments, a gene modifying system described herein is delivered to a tissue or cell from the cerebrum, cerebellum, adrenal gland, ovary, pancreas, parathyroid gland, hypophysis, testis, thyroid gland, breast, spleen, tonsil, thymus, lymph node, bone marrow, lung, cardiac muscle, esophagus, stomach, small intestine, colon, liver, salivary gland, kidney, prostate, blood, or other cell or tissue type. In some embodiments, a gene modifying system described herein is used to treat a disease, such as a cancer, inflammatory disease, infectious disease, genetic defect, or other disease. A cancer can be cancer of the cerebrum, cerebellum, adrenal gland, ovary, pancreas, parathyroid gland, hypophysis, testis, thyroid gland, breast, spleen, tonsil, thymus, lymph node, bone marrow, lung, cardiac muscle, esophagus, stomach, small intestine, colon, liver, salivary gland, kidney, prostate, blood, or other cell or tissue type, and can include multiple cancers.

In some embodiments, a gene modifying system described herein described herein is administered by enteral administration (e.g. oral, rectal, gastrointestinal, sublingual, sublabial, or buccal administration). In some embodiments, a gene modifying system described herein is administered by parenteral administration (e.g., intravenous, intramuscular, subcutaneous, intradermal, epidural, intracerebral, intracerebroventricular, epicutaneous, nasal, intra-arterial, intra-articular, intracavernous, intraocular, intraosseous infusion, intraperitoneal, intrathecal, intrauterine, intravaginal, intravesical, perivascular, or transmucosal administration). In some embodiments, a gene modifying system described herein is administered by topical administration (e.g., transdermal administration).

In some embodiments, a gene modifying system as described herein can be used to modify an animal cell, plant cell, or fungal cell. In some embodiments, a gene modifying system as described herein can be used to modify a mammalian cell (e.g., a human cell). In some embodiments, a gene modifying system as described herein can be used to modify a cell from a livestock animal (e.g., a cow, horse, sheep, goat, pig, llama, alpaca, camel, yak, chicken, duck, goose, or ostrich). In some embodiments, a gene modifying system as described herein can be used as a laboratory tool or a research tool, or used in a laboratory method or research method, e.g., to modify an animal cell, e.g., a mammalian cell (e.g., a human cell), a plant cell, or a fungal cell.

In some embodiments, a gene modifying system as described herein can be used to express a protein, template, or heterologous object sequence (e.g., in an animal cell, e.g., a mammalian cell (e.g., a human cell), a plant cell, or a fungal cell). In some embodiments, a gene modifying system as described herein can be used to express a protein, template, or heterologous object sequence under the control of an inducible promoter (e.g., a small molecule inducible promoter). In some embodiments, a gene modifying system or payload thereof is designed for tunable control, e.g., by the use of an inducible promoter. For example, a promoter, e.g., Tet, driving a gene of interest may be silent at integration, but may, in some instances, activated upon exposure to a small molecule inducer, e.g., doxycycline. In some embodiments, the tunable expression allows post-treatment control of a gene (e.g., a therapeutic gene), e.g., permitting a small molecule-dependent dosing effect. In embodiments, the small molecule-dependent dosing effect comprises altering levels of the gene product temporally and/or spatially, e.g., by local administration. In some embodiments, a promoter used in a system described herein may be inducible, e.g., responsive to an endogenous molecule of the host and/or an exogenous small molecule administered thereto.

In some embodiments, a gene modifying g system is used to make changes to non-coding and/or regulatory control regions, e.g., to tune the expression of endogenous genes. In some embodiments, a gene modifying system is used to induce upregulation or downregulation of gene expression. In some embodiments, a regulatory control region comprises one or more of a promoter, enhancer, UTR, CTCF site, and/or a gene expression control region.

In some embodiments, a gene modifying system may be used to treat or prevent a repeat expansion disease (e.g., a disease of Table 26), or to reduce the severity or a symptom thereof. In some embodiments, the repeat expansion disease comprises expansion of a trinucleotide repeat. In some embodiments, the subject has at least 10, 20, 30, 40, or 50 copies of the repeat. In embodiments, the repeat expansion disease is an inherited disease. Non-limiting examples of repeat expansion diseases include Huntington's disease (HD) and myotonic dystrophy. For example, healthy individuals may possess between 10 and 35 tandem copies of the CAG trinucleotide repeat, while Huntington's patients frequently possess >40 copies, which can result, e.g., in an elongated and dysfunctional Huntingtin protein. In some embodiments, a gene modifying system corrects a repeat expansion, e.g., by recognizing DNA at the terminus of the repeat region and nicking one strand. In some embodiments, the template RNA component of the gene modifying system comprises a region with a number of repeats characteristic of a healthy subject, e.g., about 20 repeats (e.g., between 5-10, 10-15, 15-20, 20-25, 25-30, 30-35, or 35-40 repeats). In some embodiments, a second strand nick and second strand synthesis then results in the integration of the newly copied DNA comprising a correct number of repeats (e.g., as described herein). In some embodiments, the system recognizes DNA at the terminus of the repeat region and the template carries the information for the new number of repeats. In embodiments, a gene modifying system can be used in this way regardless of the number of repeats present in an individual and/or in an individual cell. Owing to the presence of multiple repeats, an alternative non-gene modifying therapeutic (e.g., a CRISPR-based homologous recombination therapeutic) might, in some embodiments, result in unpredictable repair behavior. Further non-limiting examples of repeat expansion diseases and the causative repeats can be found, for example, in La Spada and Taylor Nat Rev Genet 11(4):247-258 (2010), which is incorporated herein by reference in its entirety.

In some embodiments, a gene modifying system may be used to treat a healthy individual, e.g., as a preventative therapy. Gene modifying systems can, in some embodiments, be targeted to generate mutations, e.g., that have been shown to be protective towards a disease of interest. An exemplary list of such diseases and protective mutation targets can be found in Table 22.

In some embodiments, a nucleic acid component of a system provided by the invention a sequence (e.g., encoding the polypeptide or comprising a heterologous object sequence) is flanked by untranslated regions (UTRs) that modify protein expression levels. Various 5′ and 3′ UTRs can affect protein expression. For example, in some embodiments, the coding sequence may be preceded by a 5′ UTR that modifies RNA stability or protein translation. In some embodiments, the sequence may be followed by a 3′ UTR that modifies RNA stability or translation. In some embodiments, the sequence may be preceded by a 5′ UTR and followed by a 3′ UTR that modify RNA stability or translation. In some embodiments, the 5′ and/or 3′ UTR may be selected from the 5′ and 3′ UTRs of complement factor 3 (C3) (cactcctccccatcctctccctctgtccctctgtccctctgaccctgcactgtcccagcacc (SEQ ID NO: 15435)) or orosomucoid 1 (ORM1) (caggacacagccttggatcaggacagagacttgggggccatcctgcccctccaacccgacatgtgtacctcagctttttccctcacttgcat caataaagcttctgtgtttggaacagctaa (SEQ ID NO: 15436)) (Asrani et al. RNA Biology 2018). In certain embodiments, the 5′ UTR is the 5′ UTR from C3 and the 3′ UTR is the 3′ UTR from ORM1.

In certain embodiments, a 5′ UTR and 3′ UTR for protein expression, e.g., mRNA (or DNA encoding the RNA) for a gene modifying polypeptide or heterologous object sequence, comprise optimized expression sequences. In some embodiments, the 5′ UTR comprises GGGAAAUAAGAGAGAAAAGAAGAGUAAGAAGAAAUAUAAGAGCCACC (SEQ ID NO: 15430) and/or the 3′ UTR comprising UGAUAAUAGGCUGGAGCCUCGGUGGCCAUGCUUCUUGCCCCUUGGGCCUCCCCCC AGCCCCUCCUCCCCUUCCUGCACCCGUACCCCCGUGGUCUUUGAAUAAAGUCUGA (SEQ ID NO: 15431), e.g., as described in Richner et al. Cell 168(6): P1114-1125 (2017), the sequences of which are incorporated herein by reference.

In some embodiments, a 5′ and/or 3′ UTR may be selected to enhance protein expression. In some embodiments, a 5′ and/or 3′ UTR may be selected to modify protein expression such that overproduction inhibition is minimized. In some embodiments, UTRs are around a coding sequence, e.g., outside the coding sequence and in other embodiments proximal to the coding sequence. In some embodiments, additional regulatory elements (e.g., miRNA binding sites, cis-regulatory sites) are included in the UTRs.

In some embodiments, an open reading frame (ORF) of a gene modifying system, e.g., an ORF of an mRNA (or DNA encoding an mRNA) encoding a gene modifying polypeptide or one or more ORFs of an mRNA (or DNA encoding an mRNA) of a heterologous object sequence, is flanked by a 5′ and/or 3′ untranslated region (UTR) that enhances the expression thereof. In some embodiments, the 5′ UTR of an mRNA component (or transcript produced from a DNA component) of the system comprises the sequence 5′-GGGAAAUAAGAGAGAAAAGAAGAGUAAGAAGAAAUAUAAGAGCCACC-3′ (SEQ ID NO: 15430). In some embodiments, the 3′ UTR of an mRNA component (or transcript produced from a DNA component) of the system comprises the sequence 5′-UGAUAAUAGGCUGGAGCCUCGGUGGCCAUGCUUCUUGCCCCUUGGGCCUCCCCCC AGCCCCUCCUCCCCUUCCUGCACCCGUACCCCCGUGGUCUUUGAAUAAAGUCUGA-3′ (SEQ ID NO: 15431). This combination of 5′ UTR and 3′ UTR has been shown to result in desirable expression of an operably linked ORF by Richner et al. Cell 168(6): P1114-1125 (2017), the teachings and sequences of which are incorporated herein by reference. In some embodiments, a system described herein comprises a DNA encoding a transcript, wherein the DNA comprises the corresponding 5′ UTR and 3′ UTR sequences, with T substituting for U in the above-listed sequence). In some embodiments, a DNA vector used to produce an RNA component of the system further comprises a promoter upstream of the 5′ UTR for initiating in vitro transcription, e.g, a T7, T3, or SP6 promoter. The 5′ UTR above begins with GGG, which is a suitable start for optimizing transcription using T7 RNA polymerase. For tuning transcription levels and altering the transcription start site nucleotides to fit alternative 5′ UTRs, the teachings of Davidson et al. Pac Symp Biocomput 433-443 (2010) describe T7 promoter variants, and the methods of discovery thereof, that fulfill both of these traits.

### Viral Vectors and Components Thereof

Viruses are a useful source of delivery vehicles for the systems described herein In some embodiments, the virus used as a gene modifying delivery system may be selected from a group as described by Baltimore Bacteriol Rev 35(3):235-241 (1971).

In some embodiments, the virus is selected from a Group I virus, e.g., is a DNA virus and packages dsDNA into virions. In some embodiments, the Group I virus is selected from, e.g., Adenoviruses, Herpesviruses, Poxviruses.

In some embodiments, the virus is selected from a Group II virus, e.g., is a DNA virus and packages ssDNA into virions. In some embodiments, the Group II virus is selected from, e.g., Parvoviruses. In some embodiments, the parvovirus is a dependoparvovirus, e.g., an adeno-associated virus (AAV).

In some embodiments, the virus is selected from a Group III virus, e.g., is an RNA virus and packages dsRNA into virions. In some embodiments, the Group III virus is selected from, e.g., Reoviruses. In some embodiments, one or both strands of the dsRNA contained in such virions is a coding molecule able to serve directly as mRNA upon transduction into a host cell, e.g., can be directly translated into protein upon transduction into a host cell without requiring any intervening nucleic acid replication or polymerization steps.

In some embodiments, the virus is selected from a Group IV virus, e.g., is an RNA virus and packages ssRNA(+) into virions. In some embodiments, the Group IV virus is selected from, e.g., Coronaviruses, Picornaviruses, Togaviruses. In some embodiments, the ssRNA(+) contained in such virions is a coding molecule able to serve directly as mRNA upon transduction into a host cell, e.g., can be directly translated into protein upon transduction into a host cell without requiring any intervening nucleic acid replication or polymerization steps.

In some embodiments, the virus is selected from a Group V virus, e.g., is an RNA virus and packages ssRNA(−) into virions. In some embodiments, the Group V virus is selected from, e.g., Orthomyxoviruses, Rhabdoviruses. In some embodiments, an RNA virus with an ssRNA(−) genome also carries an enzyme inside the virion that is transduced to host cells with the viral genome, e.g., an RNA-dependent RNA polymerase, capable of copying the ssRNA(−) into ssRNA(+) that can be translated directly by the host.

In some embodiments, the virus is selected from a Group VI virus, e.g., is a retrovirus and packages ssRNA(+) into virions. In some embodiments, the Group VI virus is selected from, e.g., Retroviruses. In some embodiments, the retrovirus is a lentivirus, e.g., HIV-1, HIV-2, SIV, BIV. In some embodiments, the retrovirus is a spumavirus, e.g., a foamy virus, e.g., HFV, SFV, BFV. In some embodiments, the ssRNA(+) contained in such virions is a coding molecule able to serve directly as mRNA upon transduction into a host cell, e.g., can be directly translated into protein upon transduction into a host cell without requiring any intervening nucleic acid replication or polymerization steps. In some embodiments, the ssRNA(+) is first reverse transcribed and copied to generate a dsDNA genome intermediate from which mRNA can be transcribed in the host cell. In some embodiments, an RNA virus with an ssRNA(+) genome also carries an enzyme inside the virion that is transduced to host cells with the viral genome, e.g., an RNA-dependent DNA polymerase, capable of copying the ssRNA(+) into dsDNA that can be transcribed into mRNA and translated by the host. In some embodiments, the reverse transcriptase from a Group VI retrovirus is incorporated as the reverse transcriptase domain of a gene modifying polypeptide.

In some embodiments, the virus is selected from a Group VII virus, e.g., is a retrovirus and packages dsRNA into virions. In some embodiments, the Group VII virus is selected from, e.g., Hepadnaviruses. In some embodiments, one or both strands of the dsRNA contained in such virions is a coding molecule able to serve directly as mRNA upon transduction into a host cell, e.g., can be directly translated into protein upon transduction into a host cell without requiring any intervening nucleic acid replication or polymerization steps. In some embodiments, one or both strands of the dsRNA contained in such virions is first reverse transcribed and copied to generate a dsDNA genome intermediate from which mRNA can be transcribed in the host cell. In some embodiments, an RNA virus with a dsRNA genome also carries an enzyme inside the virion that is transduced to host cells with the viral genome, e.g., an RNA-dependent DNA polymerase, capable of copying the dsRNA into dsDNA that can be transcribed into mRNA and translated by the host. In some embodiments, the nucleic acid in a virion may comprise DNA, e.g., linear ssDNA, linear dsDNA, circular ssDNA, circular dsDNA, minicircle DNA, dbDNA, ceDNA. In some embodiments, the nucleic acid in a virion may comprise RNA, e.g., linear ssRNA, linear dsRNA, circular ssRNA, circular dsRNA. In some embodiments, a viral genome may circularize upon transduction into a host cell, e.g., a linear ssRNA molecule may undergo a covalent linkage to form a circular ssRNA, a linear dsRNA molecule may undergo a covalent linkage to form a circular dsRNA or one or more circular ssRNA. In some embodiments, a viral genome may replicate by rolling circle replication in a host cell. In some embodiments, a viral genome may comprise a single nucleic acid molecule, e.g., comprise a non-segmented genome. In some embodiments, a viral genome may comprise two or more nucleic acid molecules, e.g., comprise a segmented genome. In some embodiments, a nucleic acid in a virion may be associated with one or proteins. In some embodiments, a virion used as a delivery vehicle may comprise a commensal human virus. In some embodiments, a virion used as a delivery vehicle may comprise an anellovirus, the use of which is described in WO2018232017A1, which is incorporated herein by reference in its entirety.

### Adeno-Associated Viruses

In some embodiments, the virus is an adeno-associated virus (AAV). In some embodiments, the AAV genome comprises two genes that encode four replication proteins and three capsid proteins, respectively. In some embodiments, the genes are flanked on either side by 145-bp inverted terminal repeats (ITRs). In some embodiments, the virion comprises up to three capsid proteins (Vp1, Vp2, and/or Vp3), e.g., produced in a 1:1:10 ratio. In some embodiments, the capsid proteins are produced from the same open reading frame and/or from differential splicing (Vp1) and alternative translational start sites (Vp2 and Vp3, respectively). Generally, Vp3 is the most abundant subunit in the virion and participates in receptor recognition at the cell surface defining the tropism of the virus. In some embodiments, Vp1 comprises a phospholipase domain, e.g., which functions in viral infectivity, in the N-terminus of Vp1.

In some embodiments, packaging capacity of the viral vectors limits the size of the base editor that can be packaged into the vector. For example, the packaging capacity of the AAVs can be about 4.5 kb (e.g., about 3.0, 3.5, 4.0, 4.5, 5.0, 5.5, or 6.0 kb), e.g., including one or two inverted terminal repeats (ITRs), e.g., 145 base ITRs.

In some embodiments, recombinant AAV (rAAV) comprises cis-acting 145-bp ITRs flanking vector transgene cassettes, e.g., providing up to 4.5 kb for packaging of foreign DNA. Subsequent to infection, rAAV can, in some instances, express an exogenous protein and persist without integration into the host genome by existing episomally in circular head-to-tail concatemers. rAAV can be used, for example, in vitro and in vivo. In some embodiments, AAV-mediated gene delivery requires that the length of the coding sequence of the gene is equal or greater in size than the wild-type AAV genome.

AAV delivery of genes that exceed this size and/or the use of large physiological regulatory elements can be accomplished, for example, by dividing the protein(s) to be delivered into two or more fragments. In some embodiments, the N-terminal fragment is fused to a split intein-N. In some embodiments, the C-terminal fragment is fused to a split intein-C. In embodiments, the fragments are packaged into two or more AAV vectors.

In some embodiments, dual AAV vectors are generated by splitting a large transgene expression cassette in two separate halves (5 and 3 ends, or head and tail), e.g., wherein each half of the cassette is packaged in a single AAV vector (of <5 kb). The re-assembly of the full-length transgene expression cassette can, in some embodiments, then be achieved upon co-infection of the same cell by both dual AAV vectors. In some embodiments, co-infection is followed by one or more of: (1) homologous recombination (HR) between 5 and 3 genomes (dual AAV overlapping vectors); (2) ITR-mediated tail-to-head concatemerization of 5 and 3 genomes (dual AAV trans-splicing vectors); and/or (3) a combination of these two mechanisms (dual AAV hybrid vectors). In some embodiments, the use of dual AAV vectors in vivo results in the expression of full-length proteins. In some embodiments, the use of the dual AAV vector platform represents an efficient and viable gene transfer strategy for transgenes of greater than about 4.0, 4.1, 4.2, 4.3, 4.4, 4.5, 4.6, 4.7, 4.8, 4.9, or 5.0 kb in size. In some embodiments, AAV vectors can also be used to transduce cells with target nucleic acids, e.g., in the in vitro production of nucleic acids and peptides. In some embodiments, AAV vectors can be used for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994); each of which is incorporated herein by reference in their entirety). The construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989) (incorporated by reference herein in their entirety).

In some embodiments, a gene modifying system described herein (e.g., with or without one or more guide nucleic acids) can be delivered using AAV, lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as described in U.S. Pat. No. 8,454,972 and as in clinical trials involving AAV. For Adenovirus, the route of administration, formulation and dose can be as described in U.S. Pat. No. 8,404,658 and as in clinical trials involving adenovirus. For plasmid delivery, the route of administration, formulation and dose can be as described in U.S. Pat. No. 5,846,946 and as in clinical studies involving plasmids. Doses can be based on or extrapolated to an average 70 kg individual (e.g. a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. In some embodiments, the viral vectors can be injected into the tissue of interest. For cell-type specific gene editing, the expression of the gene modifying polypeptide and optional guide nucleic acid can, in some embodiments, be driven by a cell-type specific promoter.

In some embodiments, AAV allows for low toxicity, for example, due to the purification method not requiring ultracentrifugation of cell particles that can activate the immune response. In some embodiments, AAV allows low probability of causing insertional mutagenesis, for example, because it does not substantially integrate into the host genome.

In some embodiments, AAV has a packaging limit of about 4.4, 4.5, 4.6, 4.7, or 4.75 kb. In some embodiments, a gene modifying polypeptide, promoter, and transcription terminator can fit into a single viral vector. SpCas9 (4.1 kb) may, in some instances, be difficult to package into AAV. Therefore, in some embodiments, a gene modifying polypeptide is used that is shorter in length than other gene modifying polypeptides or base editors. In some embodiments, the gene modifying polypeptides are less than about 4.5 kb, 4.4 kb, 4.3 kb, 4.2 kb, 4.1 kb, 4 kb, 3.9 kb, 3.8 kb, 3.7 kb, 3.6 kb, 3.5 kb, 3.4 kb, 3.3 kb, 3.2 kb, 3.1 kb, 3 kb, 2.9 kb, 2.8 kb, 2.7 kb, 2.6 kb, 2.5 kb, 2 kb, or 1.5 kb.

An AAV can be AAV1, AAV2, AAV5 or any combination thereof. In some embodiments, the type of AAV is selected with respect to the cells to be targeted; e.g., AAV serotypes 1, 2, 5 or a hybrid capsid AAV1, AAV2, AAV5 or any combination thereof can be selected for targeting brain or neuronal cells; or AAV4 can be selected for targeting cardiac tissue. In some embodiments, AAV8 is selected for delivery to the liver. Exemplary AAV serotypes as to these cells are described, for example, in Grimm, D. et al, J. Virol. 82: 5887-5911 (2008) (incorporated herein by reference in its entirety). In some embodiments, AAV refers all serotypes, subtypes, and naturally-occurring AAV as well as recombinant AAV. AAV may be used to refer to the virus itself or a derivative thereof. In some embodiments, AAV includes AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrhlO, AAVLK03, AV10, AAV11, AAV 12, rhAlO, and hybrids thereof, avian AAV, bovine AAV, canine AAV, equine AAV, primate AAV, nonprimate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. Additional exemplary AAV serotypes are listed in Table 36.

TABLE 36
Exemplary AAV serotypes.

| Target | | |
|---|---|---|
| Tissue | Vehicle | Reference |
| Liver | AAV (AAV8¹, AAVrh.8¹, | 1. Wang et al., Mol. Ther. 18, |
| | AAVhu.37¹, AAV2/8, | 118-25 (2010) |
| | AAV2/rh10², AAV9, AAV2, | 2. Ginn et al., JHEP Reports, |
| | NP40³, NP59^{2,3}, AAV3B⁵, | 100065 (2019) |
| | AAV-DJ⁴, AAV-LK01⁴, | 3. Paulk et al., Mol. Ther. 26, |
| | AAV-LK02⁴, AAV-LK03⁴, | 289-303 (2018). |
| | AAV-LK19⁴ | 4. L. Lisowski et al., Nature. |
| | Adenovirus (Ad5, HC-AdV⁶) | 506, 382-6 (2014). |
| | | 5. L. Wang et al., Mol. Ther. |
| | | 23, 1877-87 (2015). |
| | | 6. Hausl Mol Ther (2010) |
| Lung | AAV (AAV4, AAV5, | 1. Duncan et al., Mol Ther |
| | AAV6¹, AAV9, H22²) | Methods Clin Dev (2018) |
| | Adenovirus (Ad5, Ad3, | 2. Cooney et al., Am J Respir |
| | Ad21, Ad14)³ | Cell Mol Biol (2019) |
| | | 3. Li et al., Mol Ther Methods |
| | | Clin Dev (2019) |
| Skin | AAV (AAV6¹, AAV-LK19²) | 1. Petek et al., Mol. Ther. |
| | | (2010) |
| | | 2. L. Lisowski et al., Nature. |
| | | 506, 382-6 (2014). |
| HSCs | Adenovirus (HDAd5/35⁺⁺) | Wang et al. Blood Adv (2019) |

In some embodiments, a pharmaceutical composition (e.g., comprising an AA V as described herein) has less than 10% empty capsids, less than 8% empty capsids, less than 7% empty capsids, less than 5% empty capsids, less than 3% empty capsids, or less than 1% empty capsids. In some embodiments, the pharmaceutical composition has less than about 5% empty capsids. In some embodiments, the number of empty capsids is below the limit of detection. In some embodiments, it is advantageous for the pharmaceutical composition to have low amounts of empty capsids, e.g., because empty capsids may generate an adverse response (e.g., immune response, inflammatory response, liver response, and/or cardiac response), e.g., with little or no substantial therapeutic benefit.

In some embodiments, the residual host cell protein (rHCP) in the pharmaceutical composition is less than or equal to 100 ng/ml rHCP per 1×10¹³ vg/ml, e.g., less than or equal to 40 ng/ml rHCP per 1×10¹³ vg/ml or 1-50 ng/ml rHCP per 1×10¹³ vg/ml. In some embodiments, the pharmaceutical composition comprises less than 10 ng rHCP per 1.0×10¹³ vg, or less than 5 ng rHCP per 1.0×10¹³ vg, less than 4 ng rHCP per 1.0×10¹³ vg, or less than 3 ng rHCP per 1.0×10¹³ vg, or any concentration in between. In some embodiments, the residual host cell DNA (hcDNA) in the pharmaceutical composition is less than or equal to 5×10⁶ pg/ml hcDNA per 1×10¹³ vg/ml, less than or equal to 1.2×10⁶ pg/ml hcDNA per 1×10¹³ vg/ml, or 1×10⁵ μg/ml hcDNA per 1×10¹³ vg/ml. In some embodiments, the residual host cell DNA in said pharmaceutical composition is less than 5.0×10⁵ pg per 1×10¹³ vg, less than 2.0×10⁵ pg per 1.0×10¹³ vg, less than 1.1×10⁵ μg per 1.0×10¹³ vg, less than 1.0×10⁵ pg hcDNA per 1.0×10¹³ vg, less than 0.9×10⁵ pg hcDNA per 1.0×10¹³ vg, less than 0.8×10⁵ pg hcDNA per 1.0×10¹³ vg, or any concentration in between.

In some embodiments, the residual plasmid DNA in the pharmaceutical composition is less than or equal to 1.7×10⁵ pg/ml per 1.0×10¹³ vg/ml, or 1×10⁵ pg/ml per 1×1.0×10¹³ vg/ml, or 1.7×10⁶ pg/ml per 1.0×10¹³ vg/ml. In some embodiments, the residual DNA plasmid in the pharmaceutical composition is less than 10.0×10⁵ pg by 1.0×10¹³ vg, less than 8.0×10⁵ pg by 1.0×10¹³ vg or less than 6.8×10⁵ pg by 1.0×10¹³ vg. In embodiments, the pharmaceutical composition comprises less than 0.5 ng per 1.0×10¹³ vg, less than 0.3 ng per 1.0×10¹³ vg, less than 0.22 ng per 1.0×10¹³ vg or less than 0.2 ng per 1.0×10¹³ vg or any intermediate concentration of bovine serum albumin (BSA). In embodiments, the benzonase in the pharmaceutical composition is less than 0.2 ng by 1.0×10¹³ vg, less than 0.1 ng by 1.0×10¹³ vg, less than 0.09 ng by 1.0×10¹³ vg, less than 0.08 ng by 1.0×10¹³ vg or any intermediate concentration. In embodiments, Poloxamer 188 in the pharmaceutical composition is about 10 to 150 ppm, about 15 to 100 ppm or about 20 to 80 ppm. In embodiments, the cesium in the pharmaceutical composition is less than 50 pg/g (ppm), less than 30 pg/g (ppm) or less than 20 pg/g (ppm) or any intermediate concentration.

In embodiments, the pharmaceutical composition comprises total impurities, e.g., as determined by SDS-PAGE, of less than 10%, less than 8%, less than 7%, less than 6%, less than 5%, less than 4%, less than 3%, less than 2%, or any percentage in between. In embodiments, the total purity, e.g., as determined by SDS-PAGE, is greater than 90%, greater than 92%, greater than 93%, greater than 94%, greater than 95%, greater than 96%, greater than 97%, greater than 98%, or any percentage in between. In embodiments, no single unnamed related impurity, e.g., as measured by SDS-PAGE, is greater than 5%, greater than 4%, greater than 3% or greater than 2%, or any percentage in between. In embodiments, the pharmaceutical composition comprises a percentage of filled capsids relative to total capsids (e.g., peak 1+peak 2 as measured by analytical ultracentrifugation) of greater than 85%, greater than 86%, greater than 87%, greater than 88%, greater than 89%, greater than 90%, greater than 91%, greater than 91.9%, greater than 92%, greater than 93%, or any percentage in between. In embodiments of the pharmaceutical composition, the percentage of filled capsids measured in peak 1 by analytical ultracentrifugation is 20-80%, 25-75%, 30-75%, 35-75%, or 37.4-70.3%. In embodiments of the pharmaceutical composition, the percentage of filled capsids measured in peak 2 by analytical ultracentrifugation is 20-80%, 20-70%, 22-65%, 24-62%, or 24.9-60.1%.

In one embodiment, the pharmaceutical composition comprises a genomic titer of 1.0 to 5.0×10¹³ vg/mL, 1.2 to 3.0×10¹³ vg/mL or 1.7 to 2.3×10¹³ vg/ml. In one embodiment, the pharmaceutical composition exhibits a biological load of less than 5 CFU/mL, less than 4 CFU/mL, less than 3 CFU/mL, less than 2 CFU/mL or less than 1 CFU/mL or any intermediate contraction. In embodiments, the amount of endotoxin according to USP, for example, USP <85> (incorporated by reference in its entirety) is less than 1.0 EU/mL, less than 0.8 EU/mL or less than 0.75 EU/mL. In embodiments, the osmolarity of a pharmaceutical composition according to USP, for example, USP <785> (incorporated by reference in its entirety) is 350 to 450 mOsm/kg, 370 to 440 mOsm/kg or 390 to 430 mOsm/kg. In embodiments, the pharmaceutical composition contains less than 1200 particles that are greater than 25 m per container, less than 1000 particles that are greater than 25 μm per container, less than 500 particles that are greater than 25 μm per container or any intermediate value. In embodiments, the pharmaceutical composition contains less than 10,000 particles that are greater than 10 m per container, less than 8000 particles that are greater than 10 μm per container or less than 600 particles that are greater than 10 μm per container.

In one embodiment, the pharmaceutical composition has a genomic titer of 0.5 to 5.0×10¹³ vg/mL, 1.0 to 4.0×10¹³ vg/mL, 1.5 to 3.0×10¹³ vg/ml or 1.7 to 2.3×10¹³ vg/ml. In one embodiment, the pharmaceutical composition described herein comprises one or more of the following: less than about 0.09 ng benzonase per 1.0×10¹³ vg, less than about 30 pg/g (ppm) of cesium, about 20 to 80 ppm Poloxamer 188, less than about 0.22 ng BSA per 1.0×10¹³ vg, less than about 6.8×10 5 μg of residual DNA plasmid per 1.0×10¹³ vg, less than about 1.1×10⁵ μg of residual hcDNA per 1.0×10¹³ vg, less than about 4 ng of rHCP per 1.0×10¹³ vg, pH 7.7 to 8.3, about 390 to 430 mOsm/kg, less than about 600 particles that are >25 m in size per container, less than about 6000 particles that are >10 μm in size per container, about 1.7×10¹³-2.3×10¹³ vg/mL genomic titer, infectious titer of about 3.9×10⁸ to 8.4×10¹⁰ IU per 1.0×10¹³ vg, total protein of about 100-300 pg per 1.0×10¹³ vg, mean survival of >24 days in A7SMA mice with about 7.5×10¹³ vg/kg dose of viral vector, about 70 to 130% relative potency based on an in vitro cell based assay and/or less than about 5% empty capsid. In various embodiments, the pharmaceutical compositions described herein comprise any of the viral particles discussed here, retain a potency of between ±20%, between ±15%, between ±10% or within ±5% of a reference standard. In some embodiments, potency is measured using a suitable in vitro cell assay or in vivo animal model.

Additional methods of preparation, characterization, and dosing AAV particles are taught in WO2019094253, which is incorporated herein by reference in its entirety.

Additional rAAV constructs that can be employed consonant with the invention include those described in Wang et al 2019, available at: //doi.org/10.1038/s41573-019-0012-9, including Table 1 thereof, which is incorporated by reference in its entirety.

### AAV Administration

In some embodiments, an adeno-associated virus (AAV) is used in conjunction with the system, template nucleic acid, and/or polypeptide described herein. In some embodiments, an AAV is used to deliver, administer, or package the system, template nucleic acid, and/or polypeptide described herein. In some embodiments, the AAV is a recombinant AAV (rAAV).

In some embodiments, a system comprises (a) a polypeptide described herein or a nucleic acid encoding the same, (b) a template nucleic acid (e.g., template RNA) described herein, and (c) one or more first tissue-specific expression-control sequences specific to the target tissue, wherein the one or more first tissue-specific expression-control sequences specific to the target tissue are in operative association with (a), (b), or (a) and (b), wherein, when associated with (a), (a) comprises a nucleic acid encoding the polypeptide.

In some embodiments, a system described herein further comprises a first recombinant adeno-associated virus (rAAV) capsid protein; wherein the at least one of (a) or (b) is associated with the first rAAV capsid protein, wherein at least one of (a) or (b) is flanked by AAV inverted terminal repeats (ITRs).

In some embodiments, (a) and (b) are associated with the first rAAV capsid protein.

In some embodiments, (a) and (b) are on a single nucleic acid.

In some embodiments, the system further comprises a second rAAV capsid protein, wherein at least one of (a) or (b) is associated with the second rAAV capsid protein, and wherein the at least one of (a) or (b) associated with the second rAAV capsid protein is different from the at least one of (a) or (b) is associated with the first rAAV capsid protein.

In some embodiments, the at least one of (a) or (b) is associated with the first or second rAAV capsid protein is dispersed in the interior of the first or second rAAV capsid protein, which first or second rAAV capsid protein is in the form of an AAV capsid particle.

In some embodiments, the system further comprises a nanoparticle, wherein the nanoparticle is associated with at least one of (a) or (b).

In some embodiments, (a) and (b), respectively are associated with: a) a first rAAV capsid protein and a second rAAV capsid protein; b) a nanoparticle and a first rAAV capsid protein; c) a first rAAV capsid protein; d) a first adenovirus capsid protein; e) a first nanoparticle and a second nanoparticle; or f) a first nanoparticle.

Viral vectors are useful for delivering all or part of a system provided by the invention, e.g., for use in methods provided by the invention. Systems derived from different viruses have been employed for the delivery of polypeptides, nucleic acids, or transposons; for example: integrase-deficient lentivirus, adenovirus, adeno-associated virus (AAV), herpes simplex virus, and baculovirus (reviewed in Hodge et al. Hum Gene Ther 2017; Narayanavari et al. Crit Rev Biochem Mol Biol 2017; Boehme et al. Curr Gene Ther 2015).

Adenoviruses are common viruses that have been used as gene delivery vehicles given well-defined biology, genetic stability, high transduction efficiency, and ease of large-scale production (see, for example, review by Lee et al. Genes & Diseases 2017). They possess linear dsDNA genomes and come in a variety of serotypes that differ in tissue and cell tropisms. In order to prevent replication of infectious virus in recipient cells, adenovirus genomes used for packaging are deleted of some or all endogenous viral proteins, which are provided in trans in viral production cells. This renders the genomes helper-dependent, meaning they can only be replicated and packaged into viral particles in the presence of the missing components provided by so-called helper functions. A helper-dependent adenovirus system with all viral ORFs removed may be compatible with packaging foreign DNA of up to ˜37 kb (Parks et al. J Virol 1997). In some embodiments, an adenoviral vector is used to deliver DNA corresponding to the polypeptide or template component of the gene modifying system, or both are contained on separate or the same adenoviral vector. In some embodiments, the adenovirus is a helper-dependent adenovirus (HD-AdV) that is incapable of self-packaging. In some embodiments, the adenovirus is a high-capacity adenovirus (HC-AdV) that has had all or a substantial portion of endogenous viral ORFs deleted, while retaining the necessary sequence components for packaging into adenoviral particles. For this type of vector, the only adenoviral sequences required for genome packaging are noncoding sequences: the inverted terminal repeats (ITRs) at both ends and the packaging signal at the 5′-end (Jager et al. Nat Protoc 2009). In some embodiments, the adenoviral genome also comprises stuffer DNA to meet a minimal genome size for optimal production and stability (see, for example, Hausl et al. Mol Ther 2010). Adenoviruses have been used in the art for the delivery of transposons to various tissues. In some embodiments, an adenovirus is used to deliver a gene modifying system to the liver.

In some embodiments, an adenovirus is used to deliver a gene modifying system to HSCs, e.g., HDAd5/35++. HDAd5/35++ is an adenovirus with modified serotype 35 fibers that de-target the vector from the liver (Wang et al. Blood Adv 2019). In some embodiments, the adenovirus that delivers a gene modifying system to HSCs utilizes a receptor that is expressed specifically on primitive HSCs, e.g., CD46.

Adeno-associated viruses (AAV) belong to the parvoviridae family and more specifically constitute the dependoparvovirus genus. The AAV genome is composed of a linear single-stranded DNA molecule which contains approximately 4.7 kilobases (kb) and consists of two major open reading frames (ORFs) encoding the non-structural Rep (replication) and structural Cap (capsid) proteins. A second ORF within the cap gene was identified that encodes the assembly-activating protein (AAP). The DNAs flanking the AAV coding regions are two cis-acting inverted terminal repeat (ITR) sequences, approximately 145 nucleotides in length, with interrupted palindromic sequences that can be folded into energetically stable hairpin structures that function as primers of DNA replication. In addition to their role in DNA replication, the ITR sequences have been shown to be involved in viral DNA integration into the cellular genome, rescue from the host genome or plasmid, and encapsidation of viral nucleic acid into mature virions (Muzyczka, (1992) Curr. Top. Micro. Immunol. 158:97-129). In some embodiments, one or more gene modifying nucleic acid components is flanked by ITRs derived from AAV for viral packaging. See, e.g., WO2019113310.

In some embodiments, one or more components of the gene modifying system are carried via at least one AAV vector. In some embodiments, the at least one AAV vector is selected for tropism to a particular cell, tissue, organism. In some embodiments, the AAV vector is pseudotyped, e.g., AAV2/8, wherein AAV2 describes the design of the construct but the capsid protein is replaced by that from AAV8. It is understood that any of the described vectors could be pseudotype derivatives, wherein the capsid protein used to package the AAV genome is derived from that of a different AAV serotype. In some embodiments, an AAV to be employed for gene editing may be evolved for novel cell or tissue tropism as has been demonstrated in the literature (e.g., Davidsson et al. Proc Natl Acad Sci USA 2019).

In some embodiments, the AAV delivery vector is a vector which has two AAV inverted terminal repeats (ITRs) and a nucleotide sequence of interest (for example, a sequence coding for a gene modifying polypeptide or a DNA template, or both), each of said ITRs having an interrupted (or noncontiguous) palindromic sequence, i.e., a sequence composed of three segments: a first segment and a last segment that are identical when read 5′->3′ but hybridize when placed against each other, and a segment that is different that separates the identical segments. Such sequences, notably the ITRs, form hairpin structures. See, for example, WO2012123430.

Conventionally, AAV virions with capsids are produced by introducing a plasmid or plasmids encoding the rAAV or scAAV genome, Rep proteins, and Cap proteins (Grimm et al, 1998). Upon introduction of these helper plasmids in trans, the AAV genome is “rescued” (i.e., released and subsequently recovered) from the host genome, and is further encapsidated to produce infectious AAV. In some embodiments, one or more gene modifying nucleic acids are packaged into AAV particles by introducing the ITR-flanked nucleic acids into a packaging cell in conjunction with the helper functions.

In some embodiments, the AAV genome is a so called self-complementary genome (referred to as scAAV), such that the sequence located between the ITRs contains both the desired nucleic acid sequence (e.g., DNA encoding the gene modifying polypeptide or template, or both) in addition to the reverse complement of the desired nucleic acid sequence, such that these two components can fold over and self-hybridize. In some embodiments, the self-complementary modules are separated by an intervening sequence that permits the DNA to fold back on itself, e.g., forms a stem-loop. An scAAV has the advantage of being poised for transcription upon entering the nucleus, rather than being first dependent on ITR priming and second-strand synthesis to form dsDNA. In some embodiments, one or more gene modifying components is designed as an scAAV, wherein the sequence between the AAV ITRs contains two reverse complementing modules that can self-hybridize to create dsDNA.

In some embodiments, nucleic acid (e.g., encoding a polypeptide, or a template, or both) delivered to cells is closed-ended, linear duplex DNA (CELiD DNA or ceDNA). In some embodiments, ceDNA is derived from the replicative form of the AAV genome (Li et al. PLoS One 2013). In some embodiments, the nucleic acid (e.g., encoding a polypeptide, or a template DNA, or both) is flanked by ITRs, e.g., AAV ITRs, wherein at least one of the ITRs comprises a terminal resolution site and a replication protein binding site (sometimes referred to as a replicative protein binding site). In some embodiments, the ITRs are derived from an adeno-associated virus, e.g., AAV1, AAV2, AAV3, AAV4, AAV5, AAV6, AAV7, AAV8, AAV9, AAV10, AAV11, AAV12, or a combination thereof. In some embodiments, the ITRs are symmetric. In some embodiments, the ITRs are asymmetric. In some embodiments, at least one Rep protein is provided to enable replication of the construct. In some embodiments, the at least one Rep protein is derived from an adeno-associated virus, e.g., AAV1, AAV2, AAV3, AAV4, AAV5, AAV6, AAV7, AAV8, AAV9, AAV10, AAV11, AAV12, or a combination thereof. In some embodiments, ceDNA is generated by providing a production cell with (i) DNA flanked by ITRs, e.g., AAV ITRs, and (ii) components required for ITR-dependent replication, e.g., AAV proteins Rep78 and Rep52 (or nucleic acid encoding the proteins). In some embodiments, ceDNA is free of any capsid protein, e.g., is not packaged into an infectious AAV particle. In some embodiments, ceDNA is formulated into LNPs (see, for example, WO2019051289A1).

In some embodiments, the ceDNA vector consists of two self complementary sequences, e.g., asymmetrical or symmetrical or substantially symmetrical ITRs as defined herein, flanking said expression cassette, wherein the ceDNA vector is not associated with a capsid protein. In some embodiments, the ceDNA vector comprises two self-complementary sequences found in an AAV genome, where at least one ITR comprises an operative Rep-binding element (RBE) (also sometimes referred to herein as “RBS”) and a terminal resolution site (trs) of AAV or a functional variant of the RBE. See, for example, WO2019113310.

### Lipid Nanoparticles

The methods and systems provided by the invention may employ any suitable carrier or delivery modality, including, in certain embodiments, lipid nanoparticles (LNPs). Lipid nanoparticles, in some embodiments, comprise one or more ionic lipids, such as non-cationic lipids (e.g., neutral or anionic, or zwitterionic lipids); one or more conjugated lipids (such as PEG-conjugated lipids or lipids conjugated to polymers described in Table 5 of WO2019217941; incorporated herein by reference in its entirety); one or more sterols (e.g., cholesterol); and, optionally, one or more targeting molecules (e.g., conjugated receptors, receptor ligands, antibodies); or combinations of the foregoing.

Lipids that can be used in nanoparticle formations (e.g., lipid nanoparticles) include, for example those described in Table 4 of WO2019217941, which is incorporated by reference e.g., a lipid-containing nanoparticle can comprise one or more of the lipids in table 4 of WO2019217941. Lipid nanoparticles can include additional elements, such as polymers, such as the polymers described in table 5 of WO2019217941, incorporated by reference.

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (incorporated by reference), and combinations of the foregoing.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygeris et al (2020), dx.doi.org/10.1021/acs.nanolett.0c01386, incorporated herein by reference.

In some embodiments, the lipid particle comprises an ionizable lipid, a non-cationic lipid, a conjugated lipid that inhibits aggregation of particles, and a sterol. The amounts of these components can be varied independently and to achieve desired properties. For example, in some embodiments, the lipid nanoparticle comprises an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid (e.g., encoding the gene modifying polypeptide or template nucleic acid) can be varied as desired. For example, the total lipid to nucleic acid (mass or weight) ratio can be from about 10:1 to about 30:1.

In some embodiments, an ionizable lipid may be a cationic lipid, an ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s), which bear the positive charge. In some embodiments, the lipid particle comprises a cationic lipid in formulation with one or more of neutral lipids, ionizable amine-containing lipids, biodegradable alkyn lipids, steroids, phospholipids including polyunsaturated lipids, structural lipids (e.g., sterols), PEG, cholesterol and polymer conjugated lipids. In some embodiments, the cationic lipid may be an ionizable cationic lipid. An exemplary cationic lipid as disclosed herein may have an effective pKa over 6.0. In embodiments, a lipid nanoparticle may comprise a second cationic lipid having a different effective pKa (e.g., greater than the first effective pKa), than the first cationic lipid. A lipid nanoparticle may comprise between 40 and 60 mol percent of a cationic lipid, a neutral lipid, a steroid, a polymer conjugated lipid, and a therapeutic agent, e.g., a nucleic acid (e.g., RNA) described herein (e.g., a template nucleic acid or a nucleic acid encoding a gene modifying polypeptide), encapsulated within or associated with the lipid nanoparticle. In some embodiments, the nucleic acid is co-formulated with the cationic lipid. The nucleic acid may be adsorbed to the surface of an LNP, e.g., an LNP comprising a cationic lipid. In some embodiments, the nucleic acid may be encapsulated in an LNP, e.g., an LNP comprising a cationic lipid. In some embodiments, the lipid nanoparticle may comprise a targeting moiety, e.g., coated with a targeting agent. In embodiments, the LNP formulation is biodegradable. In some embodiments, a lipid nanoparticle comprising one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of an RNA molecule, e.g., template RNA and/or a mRNA encoding the gene modifying polypeptide.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Exemplary ionizable lipids that can be used in lipid nanoparticle formulations include, without limitation, those listed in Table 1 of WO2019051289, incorporated herein by reference. Additional exemplary lipids include, without limitation, one or more of the following formulae: X of US2016/0311759; I of US20150376115 or in US2016/0376224; I, 11 or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or III of US2015/0203446; I or III of US2015/0005363; I, IA, IB, IC, ID, II, IIA, IIB, IIC, IID, or III-XXIV of US2014/0308304; of US2013/0338210; I, II, III, or IV of WO2009/132131; A of US2012/01011478; I or XXXV of US2012/0027796; XIV or XVII of US2012/0058144; of US2013/0323269; I of US2011/0117125; I, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or III of US2013/0116307; I, II, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; III-3 of WO2018/081480; I-5 or I-8 of WO2020/081938; 18 or 25 of U.S. Pat. No. 9,867,888; A of US2019/0136231; II of WO2020/219876; 1 of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-O13 or 503-013 of Whitehead et al; TS-P4C2 of U.S. Pat. No. 9,708,628; I of WO2020/106946; I of WO2020/106946.

In some embodiments, the ionizable lipid is MC3 (6Z,9Z,28Z,31Z)-heptatriaconta-6,9,28,31-tetraen-19-yl-4-(dimethylamino) butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (incorporated by reference herein in its entirety). In some embodiments, the ionizable lipid is (13Z,16Z)-A,A-dimethyl-3-nonyldocosa-13, 16-dien-1-amine (Compound 32), e.g., as described in Example 11 of WO2019051289A9 (incorporated by reference herein in its entirety). In some embodiments, the ionizable lipid is Compound 6 or Compound 22, e.g., as described in Example 12 of WO2019051289A9 (incorporated by reference herein in its entirety). In some embodiments, the ionizable lipid is heptadecan-9-yl 8-((2-hydroxyethyl)(6-oxo-6-(undecyloxy)hexyl)amino)octanoate (SM-102); e.g., as described in Example 1 of U.S. Pat. No. 9,867,888 (incorporated by reference herein in its entirety). In some embodiments, the ionizable lipid is 9Z,12Z)-3-((4,4-bis(octyloxy)butanoyl)oxy)-2-((((3-(diethylamino)propoxy)carbonyl)oxy)methyl)propyl octadeca-9,12-dienoate (LP01) e.g., as synthesized in Example 13 of WO2015/095340 (incorporated by reference herein in its entirety). In some embodiments, the ionizable lipid is Di((Z)-non-2-en-1-yl) 9-((4-dimethylamino)butanoyl)oxy)heptadecanedioate (L319), e.g. as synthesized in Example 7, 8, or 9 of US2012/0027803 (incorporated by reference herein in its entirety). In some embodiments, the ionizable lipid is 1,1′-((2-(4-(2-((2-(Bis(2-hydroxydodecyl)amino)ethyl)(2-hydroxydodecyl) amino)ethyl)piperazin-1-yl)ethyl)azanediyl)bis(dodecan-2-ol) (C12-200), e.g., as synthesized in Examples 14 and 16 of WO2010/053572 (incorporated by reference herein in its entirety). In some embodiments, the ionizable lipid is; Imidazole cholesterol ester (ICE) lipid (3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate, e.g., Structure (I) from WO2020/106946 (incorporated by reference herein in its entirety).

Some non-limiting example of lipid compounds that may be used (e.g., in combination with other lipid components) to form lipid nanoparticles for the delivery of compositions described herein, e.g., nucleic acid (e.g., RNA) described herein (e.g., a template nucleic acid or a nucleic acid encoding a gene modifying polypeptide) includes,

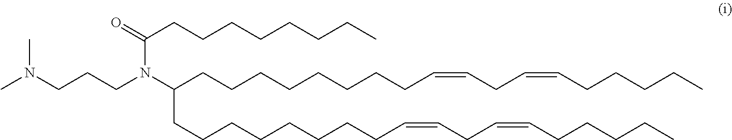 [SMILES: CCCCC/C=C\C/C=C\CCCCCCCCC(CCCCCCCC/C=C\C/C=C\CCCCC)N(CCCN(C)C)C(=O)CCCCCCCC]

In some embodiments, an LNP comprising Formula (i) is used to deliver a gene modifying composition described herein to the cells, e.g., liver and/or hepatocyte cells.

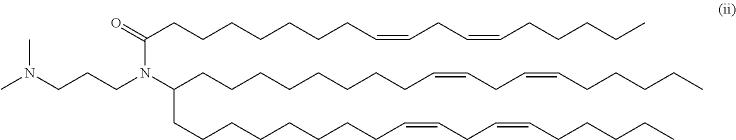 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC]

In some embodiments, an LNP comprising Formula (ii) is used to deliver a gene modifying composition described herein to the cells, e.g., liver and/or hepatocyte cells.

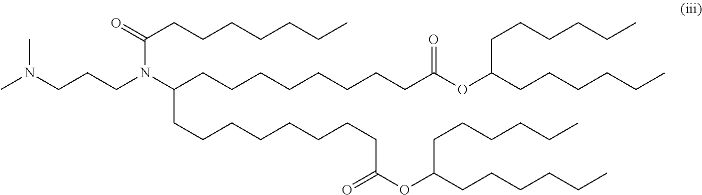 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

In some embodiments, an LNP comprising Formula (iii) is used to deliver a gene modifying composition described herein to the cells, e.g., liver and/or hepatocyte cells.

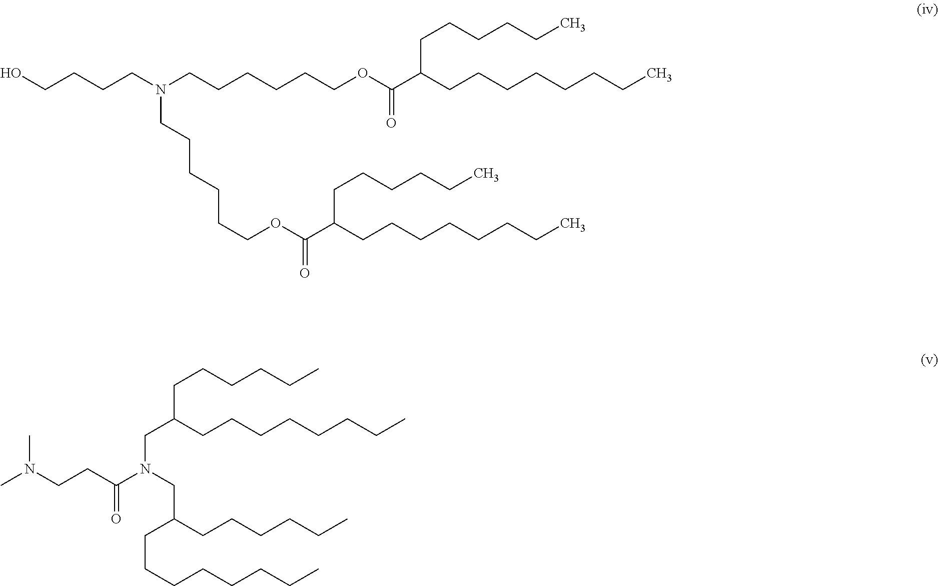

In some embodiments, an LNP comprising Formula (v) is used to deliver a gene modifying composition described herein to the cells, e.g., liver and/or hepatocyte cells.

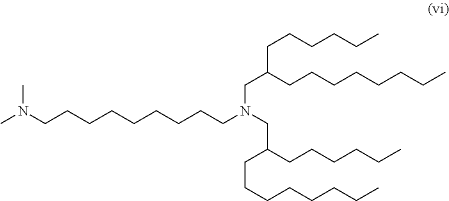 [SMILES: CCCCCCCCC(CCCCCC)CN(CCCCCCCCCN(C)C)CC(CCCCCC)CCCCCCCC]

In some embodiments, an LNP comprising Formula (vi) is used to deliver a gene modifying composition described herein to the cells, e.g., liver and/or hepatocyte cells.

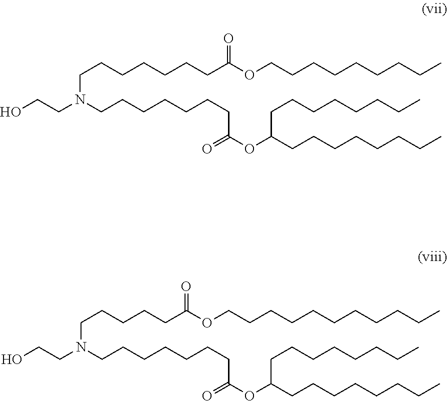 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC]

In some embodiments, an LNP comprising Formula (viii) is used to deliver a gene modifying composition described herein to the cells, e.g., liver and/or hepatocyte cells.

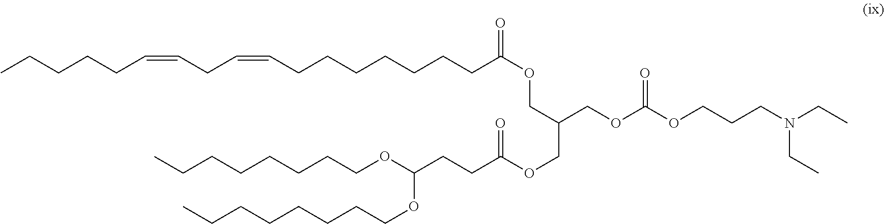 [SMILES: CCCCC/C=C\C/C=C\CCCCCCCC(=O)OCC(COC(=O)CCC(OCCCCCCCC)OCCCCCCCC)COC(=O)OCCCN(CC)CC]

In some embodiments, an LNP comprising Formula (ix) is used to deliver a gene modifying composition described herein to the cells, e.g., liver and/or hepatocyte cells.

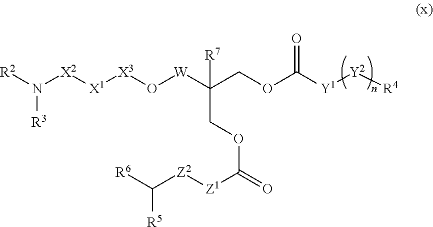

wherein:
X¹ is O, NR¹, or a direct bond, X² is C2-5 alkylene, X³ is C(═O) or a direct bond, R¹ is H or Me, R³ is Ci-3 alkyl, R² is Ci-3 alkyl, or R² taken together with the nitrogen atom to which it is attached and 1-3 carbon atoms of X² form a 4-, 5-, or 6-membered ring, or X¹ is NR¹, R¹ and R² taken together with the nitrogen atoms to which they are attached form a 5- or 6-membered ring, or R² taken together with R³ and the nitrogen atom to which they are attached form a 5-, 6-, or 7-membered ring, Y¹ is C2-12 alkylene, Y² is selected from

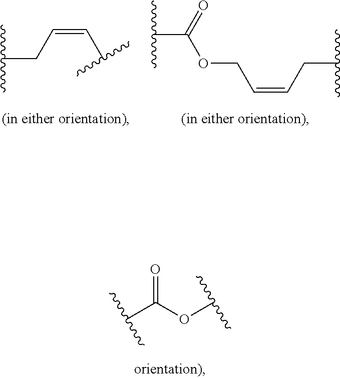 [SMILES: */C=C\C* ; *C/C=C\COC(*)=O ; *OC(*)=O]

(in either n is 0 to 3, R⁴ is Ci-15 alkyl, Z¹ is Ci-6 alkylene or a direct bond,
Z² is

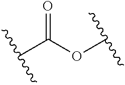 [SMILES: *OC(*)=O]

(in either orientation) or absent, provided that if Z¹ is a direct bond, Z² is absent;
R⁵ is C5-9 alkyl or C6-10 alkoxy, R⁶ is C5-9 alkyl or C6-10 alkoxy, W is methylene or a direct bond, and R⁷ is H or Me, or a salt thereof, provided that if R³ and R² are C2 alkyls, X¹ is O, X² is linear C3 alkylene, X³ is C(═O), Y¹ is linear Ce alkylene, (Y²)n-R⁴ is

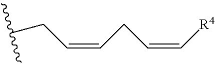

R⁴ is linear C5 alkyl, Z¹ is C2 alkylene, Z² is absent, W is methylene, and R⁷ is H, then R⁵ and R⁶ are not Cx alkoxy.

In some embodiments, an LNP comprising Formula (xii) is used to deliver a gene modifying composition described herein to the cells, e.g., liver and/or hepatocyte cells.

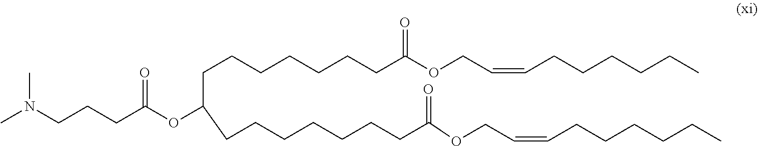 [SMILES: CCCCCC/C=C\COC(=O)CCCCCCCC(CCCCCCCC(=O)OC/C=C\CCCCCC)OC(=O)CCCN(C)C]

In some embodiments, an LNP comprising Formula (xi) is used to deliver a gene modifying composition described herein to the cells, e.g., liver and/or hepatocyte cells.

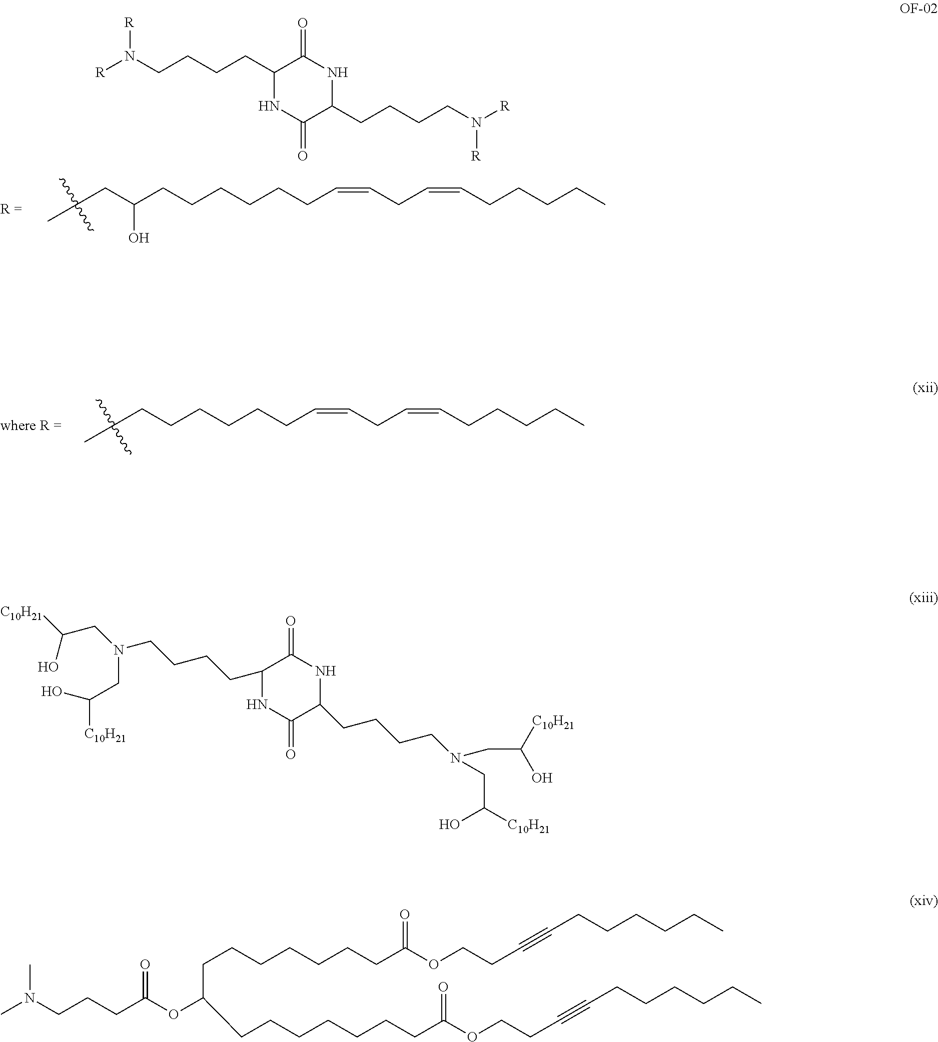

In some embodiments, an LNP comprises a compound of Formula (xiii) and a compound of Formula (xiv).

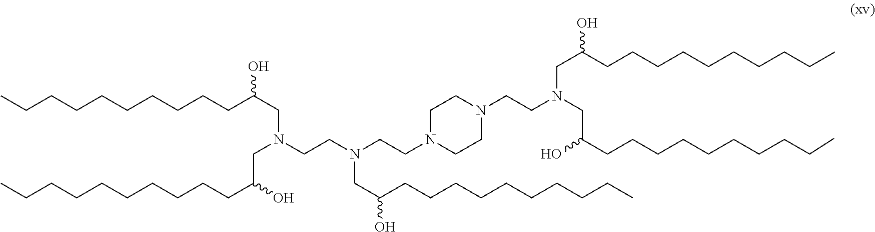 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments, an LNP comprising Formula (xv) is used to deliver a gene modifying composition described herein to the cells, e.g., liver and/or hepatocyte cells.

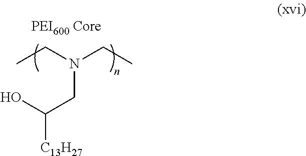

In some embodiments, an LNP comprising a formulation of Formula (xvi) is used to deliver a gene modifying composition described herein to the cells, e.g., lung endothelial cells.

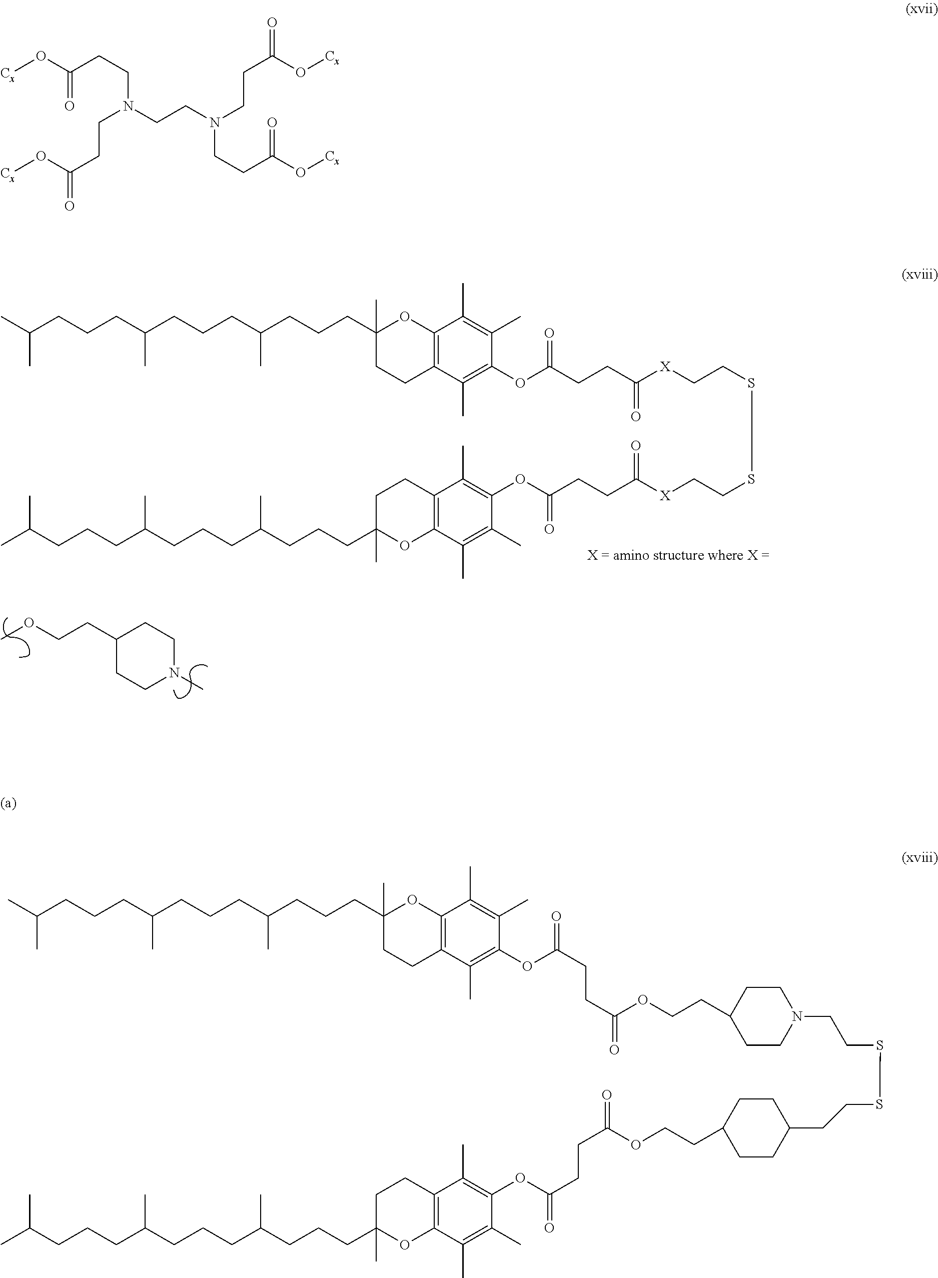

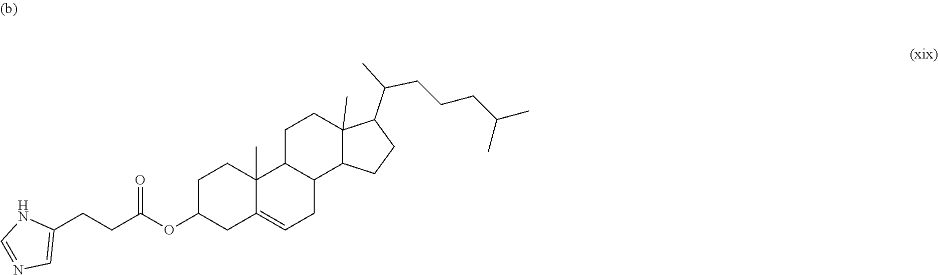 [SMILES: CC(C)CCCC(C)C1CCC2C3CC=C4CC(OC(=O)CCc5cnc[nH]5)CCC4(C)C3CCC12C]

In some embodiments, a lipid compound used to form lipid nanoparticles for the delivery of compositions described herein, e.g., nucleic acid (e.g., RNA) described herein (e.g., a template nucleic acid or a nucleic acid encoding agene modifying polypeptide) is made by one of the following reactions.

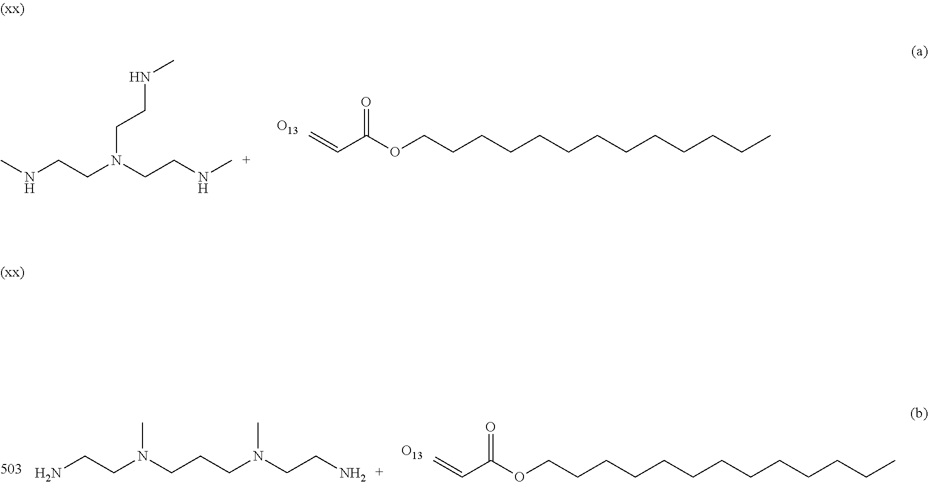

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), 1,2-dioleoyl-sn-glycero-3-phosphoethanolamine (DOPE), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, incorporated herein by reference. Such lipids include, in some embodiments, plant lipids found to improve liver transfection with mRNA (e.g., DGTS In some embodiments, the non-cationic lipid may have the following structure,

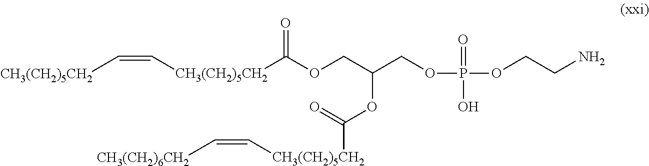

Other examples of non-cationic lipids suitable for use in the lipid nanoparticles include, without limitation, nonphosphorous lipids such as, e.g., stearylamine, dodeeylamine, hexadecylamine, acetyl palmitate, glycerol ricinoleate, hexadecyl stereate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the contents of which is incorporated herein by reference in their entirety.

In some embodiments, the non-cationic lipid is oleic acid or a compound of Formula I, II, or IV of US2018/0028664, incorporated herein by reference in its entirety. The non-cationic lipid can comprise, for example, 0-30% (mol) of the total lipid present in the lipid nanoparticle. In some embodiments, the non-cationic lipid content is 5-20% (mol) or 10-15% (mol) of the total lipid present in the lipid nanoparticle. In embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not comprise any phospholipids.

In some aspects, the lipid nanoparticle can further comprise a component, such as a sterol, to provide membrane integrity. One exemplary sterol that can be used in the lipid nanoparticle is cholesterol and derivatives thereof. Non-limiting examples of cholesterol derivatives include polar analogues such as 5a-choiestanol, 53-coprostanol, choiesteryl-(2′-hydroxy)-ethyl ether, choiesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., choiesteryl-(4′-hydroxy)-butyl ether. Exemplary cholesterol derivatives are described in PCT publication WO2009/127060 and US patent publication US2010/0130588, each of which is incorporated herein by reference in its entirety.

In some embodiments, the component providing membrane integrity, such as a sterol, can comprise 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), 1,2-dimyristoyl-sn-glycerol, methoxypoly ethylene glycol (DMG-PEG-2K), PEG succinate diacylglycerol (PEGS-DAG) (such as 4-0-(2′,3′-di(tetradecanoyloxy)propyl-l-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, and US/099823, the contents of all of which are incorporated herein by reference in their entirety. In some embodiments, a PEG-lipid is a compound of Formula III, III-a-I, III-a-2, III-b-1, III-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some embodiments, the PEG-lipid comprises PEG-DMG, 1,2-dimyristoyl-sn-glycero-3-phosphoethanolamine-N-[methoxy(polyethylene glycol)-2000]. In some embodiments, the PEG-lipid comprises a structure selected from:

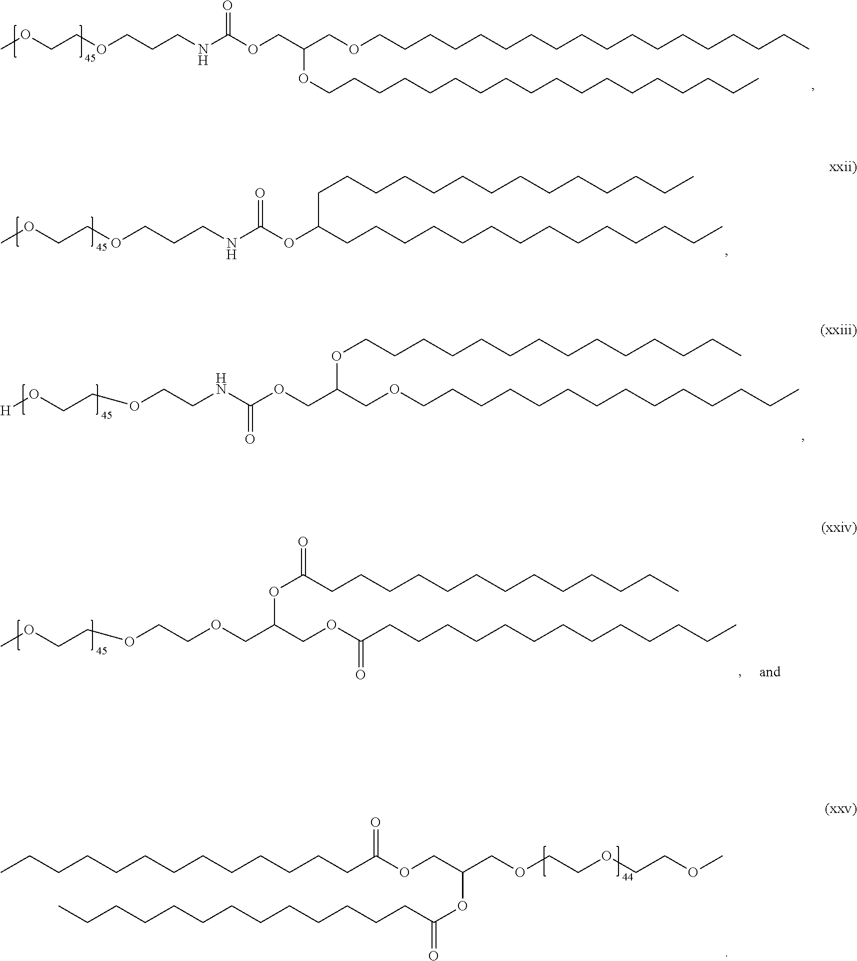

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT and LIS patent applications listed in Table 2 of WO2019051289A9 and in WO2020106946A1, the contents of all of which are incorporated herein by reference in their entirety.

In some embodiments, an LNP comprises a compound of Formula (xix), a compound of Formula (xxi) and a compound of Formula (xxv). In some embodiments a LNP comprising a formulation of Formula (xix), Formula (xxi) and Formula (xxv) is used to deliver a gene modifying composition described herein to the cells, e.g., lung or pulmonary cells.

In some embodiments, the PEG or the conjugated lipid can comprise 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can comprise 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition. Preferably, the composition comprises 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example comprising 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation comprises ionizable lipid, phospholipid, cholesterol and a PEG-ylated lipid in a molar ratio of 50:10:38.5:1.5. In some other embodiments, the lipid particle formulation comprises ionizable lipid, cholesterol and a PEG-ylated lipid in a molar ratio of 60:38.5:1.5.

In some embodiments, the lipid particle comprises ionizable lipid, non-cationic lipid (e.g. phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle comprises ionizable lipid/non-cationic-lipid/sterol/conjugated lipid at a molar ratio of 50:10:38.5:1.5.

In an aspect, the disclosure provides a lipid nanoparticle formulation comprising phospholipids, lecithin, phosphatidylcholine and phosphatidylethanolamine.

In some embodiments, one or more additional compounds can also be included. Those compounds can be administered separately or the additional compounds can be included in the lipid nanoparticles of the invention. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In some embodiments, a lipid nanoparticle (or a formulation comprising lipid nanoparticles) lacks reactive impurities (e.g., aldehydes or ketones), or comprises less than a preselected level of reactive impurities (e.g., aldehydes or ketones). While not wishing to be bound by theory, in some embodiments, a lipid reagent is used to make a lipid nanoparticle formulation, and the lipid reagent may comprise a contaminating reactive impurity (e.g., an aldehyde or ketone). A lipid regent may be selected for manufacturing based on having less than a preselected level of reactive impurities (e.g., aldehydes or ketones). Without wishing to be bound by theory, in some embodiments, aldehydes can cause modification and damage of RNA, e.g., cross-linking between bases and/or covalently conjugating lipid to RNA (e.g., forming lipid-RNA adducts). This may, in some instances, lead to failure of a reverse transcriptase reaction and/or incorporation of inappropriate bases, e.g., at the site(s) of lesion(s), e.g., a mutation in a newly synthesized target DNA.

In some embodiments, a lipid nanoparticle formulation is produced using a lipid reagent comprising less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content. In some embodiments, a lipid nanoparticle formulation is produced using a lipid reagent comprising less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species. In some embodiments, a lipid nanoparticle formulation is produced using a lipid reagent comprising: (i) less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content; and (ii) less than 5%, 4%, 3%, 2%, 1%, 0.9%, 0.8%, 0.7%, 0.6% 0.5%, 0.4%, 0.3%, 0.2%, or 0.1% of any single reactive impurity (e.g., aldehyde) species. In some embodiments, the lipid nanoparticle formulation is produced using a plurality of lipid reagents, and each lipid reagent of the plurality independently meets one or more criterion described in this paragraph. In some embodiments, each lipid reagent of the plurality meets the same criterion, e.g., a criterion of this paragraph.

In some embodiments, the lipid nanoparticle formulation comprises less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content. In some embodiments, the lipid nanoparticle formulation comprises less than 5%, 4%, 3%, 2%, 1%, 0.9%, 0.8%, 0.7%, 0.6%0, 0.5%, 0.4%, 0.3%, 0.2%, or 0.1% of any single reactive impurity (e.g., aldehyde) species. In some embodiments, the lipid nanoparticle formulation comprises: (i) less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content; and (ii) less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species.

In some embodiments, one or more, or optionally all, of the lipid reagents used for a lipid nanoparticle as described herein or a formulation thereof comprise less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content. In some embodiments, one or more, or optionally all, of the lipid reagents used for a lipid nanoparticle as described herein or a formulation thereof comprise less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species. In some embodiments, one or more, or optionally all, of the lipid reagents used for a lipid nanoparticle as described herein or a formulation thereof comprise: (i) less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content; and (ii) less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species.

In some embodiments, total aldehyde content and/or quantity of any single reactive impurity (e.g., aldehyde) species is determined by liquid chromatography (LC), e.g., coupled with tandem mass spectrometry (MS/MS), e.g., according to the method described in Example 34. In some embodiments, reactive impurity (e.g., aldehyde) content and/or quantity of reactive impurity (e.g., aldehyde) species is determined by detecting one or more chemical modifications of a nucleic acid molecule (e.g., an RNA molecule, e.g., as described herein) associated with the presence of reactive impurities (e.g., aldehydes), e.g., in the lipid reagents. In some embodiments, reactive impurity (e.g., aldehyde) content and/or quantity of reactive impurity (e.g., aldehyde) species is determined by detecting one or more chemical modifications of a nucleotide or nucleoside (e.g., a ribonucleotide or ribonucleoside, e.g., comprised in or isolated from a template nucleic acid, e.g., as described herein) associated with the presence of reactive impurities (e.g., aldehydes), e.g., in the lipid reagents. In embodiments, chemical modifications of a nucleic acid molecule, nucleotide, or nucleoside are detected by determining the presence of one or more modified nucleotides or nucleosides, e.g., using LC-MS/MS analysis.

In some embodiments, a nucleic acid (e.g., RNA) described herein (e.g., a template nucleic acid or a nucleic acid encoding a gene modifying polypeptide) does not comprise an aldehyde modification, or comprises less than a preselected amount of aldehyde modifications. In some embodiments, on average, a nucleic acid has less than 50, 20, 10, 5, 2, or 1 aldehyde modifications per 1000 nucleotides, e.g., wherein a single cross-linking of two nucleotides is a single aldehyde modification. In some embodiments, the aldehyde modification is an RNA adduct (e.g., a lipid-RNA adduct). In some embodiments, the aldehyde-modified nucleotide is cross-linking between bases. In some embodiments, a nucleic acid (e.g., RNA) described herein comprises less than 50, 20, 10, 5, 2, or 1 cross-links between nucleotides.

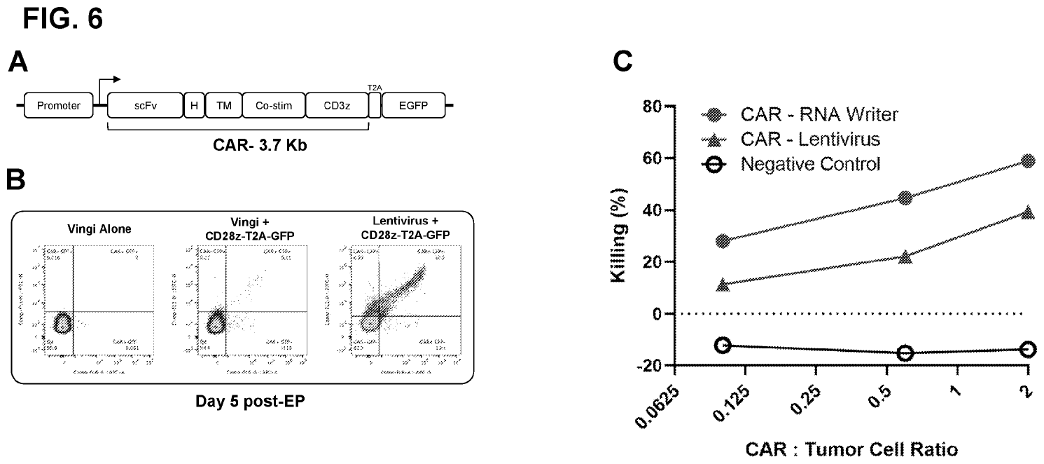

In some embodiments, LNPs are directed to specific tissues by the addition of targeting domains. For example, biological ligands may be displayed on the surface of LNPs to enhance interaction with cells displaying cognate receptors, thus driving association with and cargo delivery to tissues wherein cells express the receptor. In some embodiments, the biological ligand may be a ligand that drives delivery to the liver, e.g., LNPs that display GalNAc result in delivery of nucleic acid cargo to hepatocytes that display asialoglycoprotein receptor (ASGPR). The work of Akinc et al. Mol Ther 18(7):1357-1364 (2010) teaches the conjugation of a trivalent GalNAc ligand to a PEG-lipid (GalNAc-PEG-DSG) to yield LNPs dependent on ASGPR for observable LNP cargo effect (see, e.g., FIG. 6). Other ligand-displaying LNP formulations, e.g., incorporating folate, transferrin, or antibodies, are discussed in WO2017223135, which is incorporated herein by reference in its entirety, in addition to the references used therein, namely Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; and Peer and Lieberman, Gene Ther. 2011 18:1127-1133.

In some embodiments, LNPs are selected for tissue-specific activity by the addition of a Selective ORgan Targeting (SORT) molecule to a formulation comprising traditional components, such as ionizable cationic lipids, amphipathic phospholipids, cholesterol and poly(ethylene glycol) (PEG) lipids. The teachings of Cheng et al. Nat Nanotechnol 15(4):313-320 (2020) demonstrate that the addition of a supplemental “SORT” component precisely alters the in vivo RNA delivery profile and mediates tissue-specific (e.g., lungs, liver, spleen) gene delivery and editing as a function of the percentage and biophysical property of the SORT molecule.

In some embodiments, the LNPs comprise biodegradable, ionizable lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., lipids of WO2019/067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, multiple components of a gene modifying system may be prepared as a single LNP formulation, e.g., an LNP formulation comprises mRNA encoding for the gene modifying polypeptide and an RNA template. Ratios of nucleic acid components may be varied in order to maximize the properties of a therapeutic. In some embodiments, the ratio of RNA template to mRNA encoding a gene modifying polypeptide is about 1:1 to 100:1, e.g., about 1:1 to 20:1, about 20:1 to 40:1, about 40:1 to 60:1, about 60:1 to 80:1, or about 80:1 to 100:1, by molar ratio. In other embodiments, a system of multiple nucleic acids may be prepared by separate formulations, e.g., one LNP formulation comprising a template RNA and a second LNP formulation comprising an mRNA encoding a gene modifying polypeptide. In some embodiments, the system may comprise more than two nucleic acid components formulated into LNPs. In some embodiments, the system may comprise a protein, e.g., a gene modifying polypeptide, and a template RNA formulated into at least one LNP formulation.

In some embodiments, the average LNP diameter of the LNP formulation may be between 10s of nm and 100s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

A LNP may, in some instances, be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. In some embodiments, the zeta potential may describe the surface charge of a LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a protein and/or nucleic acid, e.g., gene modifying polypeptide or mRNA encoding the polypeptide, describes the amount of protein and/or nucleic acid that is encapsulated or otherwise associated with a LNP after preparation, relative to the initial amount provided. The encapsulation efficiency is desirably high (e.g., close to 100%). The encapsulation efficiency may be measured, for example, by comparing the amount of protein or nucleic acid in a solution containing the lipid nanoparticle before and after breaking up the lipid nanoparticle with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the lipid nanoparticles described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

A LNP may optionally comprise one or more coatings. In some embodiments, a LNP may be formulated in a capsule, film, or table having a coating. A capsule, film, or tablet including a composition described herein may have any useful size, tensile strength, hardness or density.

Additional exemplary lipids, formulations, methods, and characterization of LNPs are taught by WO2020061457, which is incorporated herein by reference in its entirety.

In some embodiments, in vitro or ex vivo cell lipofections are performed using Lipofectamine MessengerMax (Thermo Fisher) or TransIT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51(34):8529-8533 (2012), incorporated herein by reference in its entirety.

Additional specific LNP formulations useful for delivery of nucleic acids are described in U.S. Pat. Nos. 8,158,601 and 8,168,775, both incorporated by reference, which include formulations used in patisiran, sold under the name ONPATTRO.

Exemplary dosing of gene modifying composition LNP may include about 0.1, 0.25, 0.3, 0.5, 1, 2, 3, 4, 5, 6, 8, 10, or 100 mg/kg (RNA). Exemplary dosing of AAV comprising a nucleic acid encoding one or more components of the system may include an MOI of about 10¹¹, 10¹², 10¹³, and 10¹⁴ vg/kg.

Provided herein are, for example, LNP compositions comprising a gene modifying system, described herein. In some embodiments, the gene modifying polypeptide and template RNA are encapsulated within the same LNP. In some embodiments, the gene modifying polypeptide and template RNA are encapsulated in different LNPs.

### Plant-Modification Methods

Gene modifying systems described herein may be used to modify a plant or a plant part (e.g., leaves, roots, flowers, fruits, or seeds), e.g., to increase the fitness of a plant.

### A. Delivery to a Plant

Provided herein are methods of delivering a gene modifying system described herein to a plant. Included are methods for delivering a gene modifying system to a plant by contacting the plant, or part thereof, with a gene modifying system. The methods are useful for modifying the plant to, e.g., increase the fitness of a plant.

More specifically, in some embodiments, a nucleic acid described herein (e.g., a nucleic acid encoding a gene modifying polypeptide) may be encoded in a vector, e.g., inserted adjacent to a plant promoter, e.g., a maize ubiquitin promoter (ZmUBI) in a plant vector (e.g., pHUC411). In some embodiments, the nucleic acids described herein are introduced into a plant (e.g., japonica rice) or part of a plant (e.g., a callus of a plant) via agrobacteria. In some embodiments, the systems and methods described herein can be used in plants by replacing a plant gene (e.g., hygromycin phosphotransferase (HPT)) with a null allele (e.g., containing a base substitution at the start codon). Systems and methods for modifying a plant genome are described in Xu et. al. Development of plant prime-editing systems for precise genome editing, 2020, Plant Communications.

In one aspect, provided herein is a method of increasing the fitness of a plant, the method including delivering to the plant the gene modifying system described herein (e.g., in an effective amount and duration) to increase the fitness of the plant relative to an untreated plant (e.g., a plant that has not been delivered the gene modifying system).

An increase in the fitness of the plant as a consequence of delivery of a gene modifying system can manifest in a number of ways, e.g., thereby resulting in a better production of the plant, for example, an improved yield, improved vigor of the plant or quality of the harvested product from the plant, an improvement in pre- or post-harvest traits deemed desirable for agriculture or horticulture (e.g., taste, appearance, shelf life), or for an improvement of traits that otherwise benefit humans (e.g., decreased allergen production). An improved yield of a plant relates to an increase in the yield of a product (e.g., as measured by plant biomass, grain, seed or fruit yield, protein content, carbohydrate or oil content or leaf area) of the plant by a measurable amount over the yield of the same product of the plant produced under the same conditions, but without the application of the instant compositions or compared with application of conventional plant-modifying agents. For example, yield can be increased by at least about 0.5%, about 1%, about 2%, about 3%, about 4%, about 5%, about 10%, about 20%, about 30%, about 40%, about 50%, about 60%, about 70%, about 80%, about 90%, about 100%, or more than 100%. In some instances, the method is effective to increase yield by about 2x-fold, 5x-fold, 10x-fold, 25x-fold, 50x-fold, 75x-fold, 100x-fold, or more than 100x-fold relative to an untreated plant. Yield can be expressed in terms of an amount by weight or volume of the plant or a product of the plant on some basis. The basis can be expressed in terms of time, growing area, weight of plants produced, or amount of a raw material used. For example, such methods may increase the yield of plant tissues including, but not limited to: seeds, fruits, kernels, bolls, tubers, roots, and leaves.

An increase in the fitness of a plant as a consequence of delivery of a gene modifying system can also be measured by other means, such as an increase or improvement of the vigor rating, the stand (the number of plants per unit of area), plant height, stalk circumference, stalk length, leaf number, leaf size, plant canopy, visual appearance (such as greener leaf color), root rating, emergence, protein content, increased tillering, bigger leaves, more leaves, less dead basal leaves, stronger tillers, less fertilizer needed, less seeds needed, more productive tillers, earlier flowering, early grain or seed maturity, less plant verse (lodging), increased shoot growth, earlier germination, or any combination of these factors, by a measurable or noticeable amount over the same factor of the plant produced under the same conditions, but without the administration of the instant compositions or with application of conventional plant-modifying agents. Accordingly, provided herein is a method of modifying a plant, the method including delivering to the plant an effective amount of any of the gene modifying systems provided herein, wherein the method modifies the plant and thereby introduces or increases a beneficial trait in the plant (e.g., by about 1%, 2%, 5%, 10%, 20%, 30%, 40%, 50%, 60%, 70%, 80%, 90%, 100%, or more than 100%) relative to an untreated plant. In particular, the method may increase the fitness of the plant (e.g., by about 1%, 2%, 5%, 10%, 20%, 30%, 40%, 50%, 60%, 70%, 80%, 90%, 100%, or more than 100%) relative to an untreated plant.

In some instances, the increase in plant fitness is an increase (e.g., by about 1%, 2%, 5%, 10%, 20%, 30%, 40%, 50%, 60%, 70%, 80%, 90%, 100%, or more than 100%) in disease resistance, drought tolerance, heat tolerance, cold tolerance, salt tolerance, metal tolerance, herbicide tolerance, chemical tolerance, water use efficiency, nitrogen utilization, resistance to nitrogen stress, nitrogen fixation, pest resistance, herbivore resistance, pathogen resistance, yield, yield under water-limited conditions, vigor, growth, photosynthetic capability, nutrition, protein content, carbohydrate content, oil content, biomass, shoot length, root length, root architecture, seed weight, or amount of harvestable produce.

In some instances, the increase in fitness is an increase (e.g., by about 1%, 2%, 5%, 10%, 20%, 30%, 40%, 50%, 60%, 70%, 80%, 90%, 100%, or more than 100%) in development, growth, yield, resistance to abiotic stressors, or resistance to biotic stressors. An abiotic stress refers to an environmental stress condition that a plant or a plant part is subjected to that includes, e.g., drought stress, salt stress, heat stress, cold stress, and low nutrient stress. A biotic stress refers to an environmental stress condition that a plant or plant part is subjected to that includes, e.g. nematode stress, insect herbivory stress, fungal pathogen stress, bacterial pathogen stress, or viral pathogen stress. The stress may be temporary, e.g. several hours, several days, several months, or permanent, e.g. for the life of the plant.

In some instances, the increase in plant fitness is an increase (e.g., by about 1%, 2%, 5%, 10%, 20%, 30%, 40%, 50%, 60%, 70%, 80%, 90%, 100%, or more than 100%) in quality of products harvested from the plant. For example, the increase in plant fitness may be an improvement in commercially favorable features (e.g., taste or appearance) of a product harvested from the plant. In other instances, the increase in plant fitness is an increase in shelf-life of a product harvested from the plant (e.g., by about 1%, 2%, 5%, 10%, 20%, 30%, 40%, 50%, 60%, 70%, 80%, 90%, 100%, or more than 100%).

Alternatively, the increase in fitness may be an alteration of a trait that is beneficial to human or animal health, such as a reduction in allergen production. For example, the increase in fitness may be a decrease (e.g., by about 1%, 2%, 5%, 10%, 20%, 30%, 40%, 50%, 60%, 70%, 80%, 90%, 100%, or more than 100%) in production of an allergen (e.g., pollen) that stimulates an immune response in an animal (e.g., human).

The modification of the plant (e.g., increase in fitness) may arise from modification of one or more plant parts. For example, the plant can be modified by contacting leaf, seed, pollen, root, fruit, shoot, flower, cells, protoplasts, or tissue (e.g., meristematic tissue) of the plant. As such, in another aspect, provided herein is a method of increasing the fitness of a plant, the method including contacting pollen of the plant with an effective amount of any of the plant-modifying compositions herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In yet another aspect, provided herein is a method of increasing the fitness of a plant, the method including contacting a seed of the plant with an effective amount of any of the gene modifying systems disclosed herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In another aspect, provided herein is a method including contacting a protoplast of the plant with an effective amount of any of the gene modifying systems described herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In a further aspect, provided herein is a method of increasing the fitness of a plant, the method including contacting a plant cell of the plant with an effective amount of any of the gene modifying system described herein, wherein the method increases the fitness of the plant (e.g., by about 1%, 2%, 5%, 10%, 20% 30%, 40%, 50%, 60%, 70%, 80%, 90%, 100%, or more than 100%) relative to an untreated plant.

In another aspect, provided herein is a method of increasing the fitness of a plant, the method including contacting meristematic tissue of the plant with an effective amount of any of the plant-modifying compositions herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In another aspect, provided herein is a method of increasing the fitness of a plant, the method including contacting an embryo of the plant with an effective amount of any of the plant-modifying compositions herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

### B. Application Methods

A plant described herein can be exposed to any of the gene modifying system compositions described herein in any suitable manner that permits delivering or administering the composition to the plant. The gene modifying system may be delivered either alone or in combination with other active (e.g., fertilizing agents) or inactive substances and may be applied by, for example, spraying, injection (e.g,. microinjection), through plants, pouring, dipping, in the form of concentrated liquids, gels, solutions, suspensions, sprays, powders, pellets, briquettes, bricks and the like, formulated to deliver an effective concentration of the plant-modifying composition. Amounts and locations for application of the compositions described herein are generally determined by the habitat of the plant, the lifecycle stage at which the plant can be targeted by the plant-modifying composition, the site where the application is to be made, and the physical and functional characteristics of the plant-modifying composition.

In some instances, the composition is sprayed directly onto a plant, e.g., crops, by e.g., backpack spraying, aerial spraying, crop spraying/dusting etc. In instances where the gene modifying system is delivered to a plant, the plant receiving the gene modifying system may be at any stage of plant growth. For example, formulated plant-modifying compositions can be applied as a seed coating or root treatment in early stages of plant growth or as a total plant treatment at later stages of the crop cycle. In some instances, the plant-modifying composition may be applied as a topical agent to a plant.

Further, the gene modifying system may be applied (e.g., in the soil in which a plant grows, or in the water that is used to water the plant) as a systemic agent that is absorbed and distributed through the tissues of a plant. In some instances, plants or food organisms may be genetically transformed to express the gene modifying system.

Delayed or continuous release can also be accomplished by coating the gene modifying system or a composition with the plant-modifying composition(s) with a dissolvable or bioerodable coating layer, such as gelatin, which coating dissolves or erodes in the environment of use, to then make the plant-modifying gene modifying system position available, or by dispersing the agent in a dissolvable or erodable matrix. Such continuous release and/or dispensing means devices may be advantageously employed to consistently maintain an effective concentration of one or more of the plant-modifying compositions described herein.

In some instances, the gene modifying system is delivered to a part of the plant, e.g., a leaf, seed, pollen, root, fruit, shoot, or flower, or a tissue, cell, or protoplast thereof. In some instances, the gene modifying system is delivered to a cell of the plant. In some instances, the gene modifying system is delivered to a protoplast of the plant. In some instances, the gene modifying system is delivered to a tissue of the plant. For example, the composition may be delivered to meristematic tissue of the plant (e.g., apical meristem, lateral meristem, or intercalary meristem). In some instances, the composition is delivered to permanent tissue of the plant (e.g., simple tissues (e.g., parenchyma, collenchyma, or sclerenchyma) or complex permanent tissue (e.g., xylem or phloem)). In some instances, the gene modifying system is delivered to a plant embryo.

### C. Plants

A variety of plants can be delivered to or treated with a gene modifying system described herein. Plants that can be delivered a gene modifying system (i.e., “treated”) in accordance with the present methods include whole plants and parts thereof, including, but not limited to, shoot vegetative organs/structures (e.g., leaves, stems and tubers), roots, flowers and floral organs/structures (e.g., bracts, sepals, petals, stamens, carpels, anthers and ovules), seed (including embryo, endosperm, cotyledons, and seed coat) and fruit (the mature ovary), plant tissue (e.g., vascular tissue, ground tissue, and the like) and cells (e.g., guard cells, egg cells, and the like), and progeny of same. Plant parts can further refer parts of the plant such as the shoot, root, stem, seeds, stipules, leaves, petals, flowers, ovules, bracts, branches, petioles, internodes, bark, pubescence, tillers, rhizomes, fronds, blades, pollen, stamen, and the like. The class of plants that can be treated in a method disclosed herein includes the class of higher and lower plants, including angiosperms (monocotyledonous and dicotyledonous plants), gymnosperms, ferns, horsetails, psilophytes, lycophytes, bryophytes, and algae (e.g., multicellular or unicellular algae). Plants that can be treated in accordance with the present methods further include any vascular plant, for example monocotyledons or dicotyledons or gymnosperms, including, but not limited to alfalfa, apple, Arabidopsis, banana, barley, canola, castor bean, chrysanthemum, clover, cocoa, coffee, cotton, cottonseed, corn, crambe, cranberry, cucumber, dendrobium, dioscorea, eucalyptus, fescue, flax, gladiolus, liliacea, linseed, millet, muskmelon, mustard, oat, oil palm, oilseed rape, papaya, peanut, pineapple, ornamental plants, Phaseolus, potato, rapeseed, rice, rye, ryegrass, safflower, sesame, sorghum, soybean, sugarbeet, sugarcane, sunflower, strawberry, tobacco, tomato, turfgrass, wheat and vegetable crops such as lettuce, celery, broccoli, cauliflower, cucurbits; fruit and nut trees, such as apple, pear, peach, orange, grapefruit, lemon, lime, almond, pecan, walnut, hazel; vines, such as grapes (e.g., a vineyard), kiwi, hops; fruit shrubs and brambles, such as raspberry, blackberry, gooseberry; forest trees, such as ash, pine, fir, maple, oak, chestnut, popular; with alfalfa, canola, castor bean, corn, cotton, crambe, flax, linseed, mustard, oil palm, oilseed rape, peanut, potato, rice, safflower, sesame, soybean, sugarbeet, sunflower, tobacco, tomato, and wheat. Plants that can be treated in accordance with the methods of the present invention include any crop plant, for example, forage crop, oilseed crop, grain crop, fruit crop, vegetable crop, fiber crop, spice crop, nut crop, turf crop, sugar crop, beverage crop, and forest crop. In certain instances, the crop plant that is treated in the method is a soybean plant. In other certain instances, the crop plant is wheat. In certain instances, the crop plant is corn. In certain instances, the crop plant is cotton. In certain instances, the crop plant is alfalfa. In certain instances, the crop plant is sugarbeet. In certain instances, the crop plant is rice. In certain instances, the crop plant is potato. In certain instances, the crop plant is tomato.

In certain instances, the plant is a crop. Examples of such crop plants include, but are not limited to, monocotyledonous and dicotyledonous plants including, but not limited to, fodder or forage legumes, ornamental plants, food crops, trees, or shrubs selected from Acer spp., Allium spp., Amaranthus spp., Ananas comosus, Apium graveolens, Arachis spp, Asparagus officinalis, Beta vulgaris, Brassica spp. (e.g., Brassica napus, Brassica rapa ssp. (canola, oilseed rape, turnip rape), Camellia sinensis, Canna indica, Cannabis saliva, Capsicum spp., Castanea spp., Cichorium endivia, Citrullus lanatus, Citrus spp., Cocos spp., Coffea spp., Coriandrum sativum, Corylus spp., Crataegus spp., Cucurbita spp., Cucumis spp., Daucus carota, Fagus spp., Ficus carica, Fragaria spp., Ginkgo biloba, Glycine spp. (e.g., Glycine max, Soja hispida or Soja max), Gossypium hirsutum, Helianthus spp. (e.g., Helianthus annuus), Hibiscus spp., Hordeum spp. (e.g., Hordeum vulgare), Ipomoea batatas, Juglans spp., Lactuca sativa, Linum usitatissimum, Litchi chinensis, Lotus spp., Luffa acutangula, Lupinus spp., Lycopersicon spp. (e.g., Lycopersicon esculenturn, Lycopersicon lycopersicum, Lycopersicon pyriforme), Malus spp., Medicago sativa, Mentha spp., Miscanthus sinensis, Morus nigra, Musa spp., Nicotiana spp., Olea spp., Oryza spp. (e.g., Oryza sativa, Oryza latifolia), Panicum miliaceum, Panicum virgatum, Passiflora edulis, Petroselinum crispum, Phaseolus spp., Pinus spp., Pistacia vera, Pisum spp., Poa spp., Populus spp., Prunus spp., Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis spp., Solanum spp. (e.g., Solanum tuberosum, Solanum integrifolium or Solanum lycopersicum), Sorghum bicolor, Sorghum halepense, Spinacia spp., Tamarindus indica, Theobroma cacao, Trifolium spp., Triticosecale rimpaui, Triticum spp. (e.g., Triticum aestivum, Triticum durum, Triticum turgidum, Triticum hybernum, Triticum macha, Triticum sativum or Triticum vulgare), Vaccinium spp., Vicia spp., Vigna spp., Viola odorata, Vitis spp., and Zea mays. In certain embodiments, the crop plant is rice, oilseed rape, canola, soybean, corn (maize), cotton, sugarcane, alfalfa, sorghum, or wheat. The plant or plant part for use in the present invention include plants of any stage of plant development. In certain instances, the delivery can occur during the stages of germination, seedling growth, vegetative growth, and reproductive growth. In certain instances, delivery to the plant occurs during vegetative and reproductive growth stages. In some instances, the composition is delivered to pollen of the plant. In some instances, the composition is delivered to a seed of the plant. In some instances, the composition is delivered to a protoplast of the plant. In some instances, the composition is delivered to a tissue of the plant. For example, the composition may be delivered to meristematic tissue of the plant (e.g., apical meristem, lateral meristem, or intercalary meristem). In some instances, the composition is delivered to permanent tissue of the plant (e.g., simple tissues (e.g., parenchyma, collenchyma, or sclerenchyma) or complex permanent tissue (e.g., xylem or phloem)). In some instances, the composition is delivered to a plant embryo. In some instances, the composition is delivered to a plant cell. The stages of vegetative and reproductive growth are also referred to herein as “adult” or “mature” plants.

In instances where the gene modifying system is delivered to a plant part, the plant part may be modified by the plant-modifying agent. Alternatively, the gene modifying system may be distributed to other parts of the plant (e.g., by the plant's circulatory system) that are subsequently modified by the plant-modifying agent.

All publications, patent applications, patents, and other publications and references (e.g., sequence database reference numbers) cited herein are incorporated by reference in their entirety. For example, all GenBank, Unigene, and Entrez sequences referred to herein, e.g., in any Table herein, are incorporated by reference. Unless otherwise specified, the sequences specified herein (e.g., by gene name in RepBase or by accession number), including in any Table herein, refer to the database entries current as of Apr. 28, 2022. When one gene or protein references a plurality of sequence accession numbers, all of the sequence variants are encompassed.

### EXAMPLES

The invention is further illustrated by the following examples. The examples are provided for illustrative purposes only and are not to be construed as limiting the scope or content of the invention in any way.

### Example 1: Retrotransposon-Based Gene Modifying Systems Yield a Different Integration Profile than a Lentiviral System

This example characterizes the integration profile resulting from use of a gene modifying system described herein, and contrasts it to the integration profile resulting from use of a lentiviral system.

The first gene modifying system used in this Example comprises a nucleic acid encoding a Vingi-1_Acar gene modifying polypeptide (or driver) having a nucleic acid sequence of:

(SEQ ID NO: 900)
ATGGACGAGTACCAGCGCTCCTTGTCCCGCCCCCTGTTGACAATCATGAGTATTAAT
ATCGAAGGCTTGAGCCTCGCGAAAGAGGAACTGCTTGCCAAGATGTCCGAGGACAT
CAGCTGCGACATTCTGTGCATCCAAGAGACCCATCGCGACATCACCATGCGTCGCCC
CAAGATCCTCGGTATGCAGCTTGCGGTGGAGCGCCCTCACCGCCAGTACGGCTCCGC
GATTTTCGTGCGTTCCGGTGTGGCCATTTCTGCTACCAGCCTCACCGAGGTTAACAAT
ATTGAGATCCTGTCAGTGGAGCTCGATTCCTGTACCGTCTCCAGTCTGTACAAACCT
CCCGGTGCAGACTTCTACTTTACTCCCCCGACGTCTTGCCACAACCATGAGGCTCAT
TTTGTGGTTGGTGATTTCAATTCCCACTCCTGCGTGTGGGGCTATGACGAGGATGAC
CGCAACGGCGAAGCTGTTCTGACCTGGGCCGATAATTCCCGCATGTCCCTGCTCCAC
GATAGTAAGCTGCCTCCCTCCTTCAACAGTGGGAGATGGAAGCGCGGGTACAACCC
TGATCTTATCTTCGTCAAAGAATCCATCTCTCATCAGTGTACCAAGCGCGTCCTCAAC
CCGATTCCTAACACTCAGCACCGCCCCATTTGTTGCGTAGCATATGCTGCCGTGCGT
CCCAAGTCTGTCCCCTTCAGAAGGAGATACAACTTCAACAAGGCTAACTGGACCAA
GTTTACCGAAACCTTGGAAGCGGCAATCTCCGACATTGAGCCATCTATCGAGAATTA
TGATCTCTTCGTTGAGGCAGTCAAGCGGTCCTCTCGCCTGAGTATCCCCCGTGGATG
TAGGACCTCCTACCTGCCCGGCCTGAACGAAGAGTCACTCAATCAGCTGCAGGAAT
ACCTGCGCCTGTTCCAAGAGAATCCTTACTCCGATGGCACCATCGCTGCCGGACAAA
AACTGAGCACCGCTCTGGCGAATGCTAAGAAAGACCGCTGGATCGAGCTGCTCGAG
AACCTGGACATGTCCAAGTCTAGCCGGAAAGCTTGGCAGCTCCTGCGCCGGCTGGA
CAGCGACCCCCTCGTGAATCCTGGCCATGCCAATGTAACGCCGGACCAGATCGCCC
ATCAACTGATCCAGAACGGCAAGACCAATTGCAGTAGAATCAAGATGAAGATCAAC
AGAGTACCCGAGTTGGAAACACACCAGCTGTCTAGTCCTCTCAACCTCAAAGAGCTC
CGCGAAGCAATCAAAAGGTGTAAGACGGGCAAAGCTCCCGGTCTGGATGACCTGAT
GATGGAACAGATCAAACACTTGGGGGCCAAGGCTGAGAACTGGTTGCTGAAATTCT
ACAACCAGTGCCTGGCTCACAAGCAGATCCCCCGTGCGTGGCGCAAGACTAAGATT
ATCGCCATCTTGAAGCCGGGCAAGGATGCTAGTAACGCCCGCAACTACCGTCCTATT
TCCCTCCTGTGTCACCTTTACAAGGTGTACGAGCGCATGCTCCTGAACCGCTTGGGC
CCCGTCATCGAGCCTAAACTGATCGCCCAACAGGCTGGTTTCCGCCCAGGTAAGAAC
TGCACTGGACAGATCCTGCACCTTACGGAGCACATCGAGGAAGGTTACGAGAAGGG
CTGCATCACCGGAACCGTCTTCGTGGACCTGACTGCGGCCTACGACACGGTTCAGCA
CAGAAAAATGCTCCACAAGGTATATCATATTACCCGGGATTTCGACTTCACCAAAAC
AGTGCAGACGCTTCTGGAGAATCGCTCCTTTTACGTCGAGTTCCAGGGTCAGAAAAG
TCGGTGGAGGCGCCAGAAGAACGGCTTGCCCCAGGGTAGCGTGCTCGCCCCAACCC
TGTTTAATATCTTCACCAACGACCAGCCACAGCCCCCGCTGACAAAATCCTTTATCT
ATGCCGACGATCTTGGCCTGACCACGCAAGCCAAGGACTTTGAGACAGTCGAGAAA
CAGCTCACAAACGCACTCAAGGACCTGAGTTCCTATTACAAGGAGAACCATCTCAA
ACCTAACCCCGCTAAGACACAGGTCTGCGCTTTTCACCTGAGGAATCGGGAGGCCA
ACCGTAAACTGAAGGTGACATGGGAGGGACAGGAACTGGAGCACTGTTTCCACCCC
AAGTATTTGGGCGTGACTCTCGATCGCACCCTTACGTATCGGAAGCACTGCATGAAC
ACTAAGCATAAGGTGGCGGCCAGAAATAACATCCTGCGTAAACTCACAGGAAGCGC
CTGGGGCGCCGACCCACAGGTCATCCGTACCAGTGCTCTGGCCCTGTCTTTCTCCAC
TGCTGAATATGCGTGTCCGGTGTGGCACAAGTCCGCCCACGCGAAGCAGGTGGATA
TTGCCCTGAACGAGACATGCCGGATTATCACTGGTTGCCTCAAGCCTACCCCCGTCG
ACAAGCTCTATAAGTTGGCCGGCATTGCACCCCCTGATGTCCGTCGCGAAGTCGCAG
CCAATGGCGAGCGGAAAAAGGTAGAACACTGCGAATCACATCCGCTGCATGGATAT
CATCCCCCTCCCACCCGGCTTAAGAGCCGTAAGGGCTTCATGCGCACAACCACTCCG
CTGGATGTGCCTCCCGCTGCCGCGCGCGTGAGTCTGTGGGCCGCTAAGCCCGGTAAT
TCCAATTGGATGGCACCGCAAGAGGGCCTCCCGCCCGGAGCCAATCAAGAGTGGGC
CACATGGAAGTCCTTGAACAGGCTGCGCTCCGGTGTGGGACGCTCTAAGGACAACC
TGGCCCGTTGGCACTATCTGGAAGAGTCATCCACCCTGTGCGACTGTGGTGCTGAAC
AGACTACCCAGCACATGTACGCTTGCCCCCAATGCCCTGCCTCCTGCACGGAAGAGG
AACTCTTTAAGGCAACCGATAACGCAGTGGCAGTGGCGCGCTTTTGGAGTAAGACA
ATC

The Vingi driver encoded by the nucleic acid has an amino acid sequence of:

(SEQ ID NO: 405)
MDEYQRSLSRPLLTIMSINIEGLSLAKEELLAKMSEDISCDILCIQETHRDITMRRPKILGM
QLAVERPHRQYGSAIFVRSGVAISATSLTEVNNIEILSVELDSCTVSSLYKPPGADFYFTPP
TSCHNHEAHFVVGDFNSHSCVWGYDEDDRNGEAVLTWADNSRMSLLHDSKLPPSFNS
GRWKRGYNPDLIFVKESISHQCTKRVLNPIPNTQHRPICCVAYAAVRPKSVPFRRRYNFN
KANWTKFTETLEAAISDIEPSIENYDLFVEAVKRSSRLSIPRGCRTSYLPGLNEESLNQLQ
EYLRLFQENPYSDGTIAAGQKLSTALANAKKDRWIELLENLDMSKSSRKAWQLLRRLD
SDPLVNPGHANVTPDQIAHQLIQNGKTNCSRIKMKINRVPELETHQLSSPLNLKELREAIK
RCKTGKAPGLDDLMMEQIKHLGAKAENWLLKFYNQCLAHKQIPRAWRKTKIIAILKPG
KDASNARNYRPISLLCHLYKVYERMLLNRLGPVIEPKLIAQQAGFRPGKNCTGQILHLTE
HIEEGYEKGCITGTVFVDLTAAYDTVQHRKMLHKVYHITRDFDFTKTVQTLLENRSFYV
EFQGQKSRWRRQKNGLPQGSVLAPTLFNIFTNDQPQPPLTKSFIYADDLGLTTQAKDFET
VEKQLTNALKDLSSYYKENHLKPNPAKTQVCAFHLRNREANRKLKVTWEGQELEHCF
HPKYLGVTLDRTLTYRKHCMNTKHKVAARNNILRKLTGSAWGADPQVIRTSALALSFS
TAEYACPVWHKSAHAKQVDIALNETCRIITGCLKPTPVDKLYKLAGIAPPDVRREVAAN
GERKKVEHCESHPLHGYHPPPTRLKSRKGFMRTTTPLDVPPAAARVSLWAAKPGNSNW
MAPQEGLPPGANQEWATWKSLNRLRSGVGRSKDNLARWHYLEESSTLCDCGAEQTTQ
HMYACPQCPASCTEEELFKATDNAVAVARFWSKTI

The Vingi-1_Acar gene modifying system further comprises a template RNA having a sequence of:

(SEQ ID NO: 1000)
AGGGGGACACGGAAAGAGCCTCCCCGAAGATTGAGTGAATTCAGTCGGGCGTCCCC
TGGGCAACGTTTCTTGTAAGCGGCCGATCTTTCCACCCCAAAAGCATTGGATGAGTT
TACGGATCCGAATTCTCGACGGATCGATCCGAACAAACGACCCAACACCCGTGCGT
TTTATTCTGTCTTTTTATTGCCGATCCCCCGGCCGCTTTACTTGTACAGCTCGTCCATG
CCGAGAGTGATCCCGGCGGCGGTCACGAACTCCAGCAGGACCATGTGATCGCGCTT
CTCGTTGGGGTCTTTGCTCAGGGCGGACTGGGTGCTCAGGTAGTGGTTGTCGGGCAG
CAGCACGGGGCCGTCGCCGATGGGGGTGTTCTGCTGGTAGTGGTCGGCGAGCTGCA
CGCTGCCGTCCTCGATGTTGTGGCGGATCTTGAAGTTCACCTTGATGCCGTTCTTCTG
CTTGTCGGCCATGATATAGACGTTGTGGCTGTTGTAGTTGTACTCCAGCTTGTGCCCC
AGGATGTTGCCGTCCTCCTTGAAGTCGATGCCCTTCAGCTCGATGCGGTTCACCAGG
GTGTCGCCCTCGAACTTCACCTCGGCGCGGGTCTTGTAGTTGCCGTCGTCCTTGAAG
AAGATGGTGCGCTCCTGGACGTAGCCTTCGGGCATGGCGGACTTGAAGAAGTCGTG
CTGCTTCATGTGGTCGGGGTAGCGGCTGAAGCACTGCACGCCGTAGGTCAGGGTGGT
CACGAGGGTGGGCCAGGGCACGGGCAGCTTGCCGGTGGTGCAGATGAACTTCAGGG
TCAGCTTGCCGTAGGTGGCATCGCCCTCGCCCTCGCCGGACACGCTGAACTTGTGGC
CGTTTACGTCGCCGTCCAGCTCGACCAGGATGGGCACCACCCCGGTGAACAGCTCCT
CGCCCTTGCTCACCATGGTGGCTTTACCAACAGTACCGGAATGCCAAGCTTGGGTCC
TGTGTTCTGGCGGCAAACCCGTTGCGAAAAAGAACGTTCACGGCGACTACTGCACTT
ATATACGGTTCTCCCCCACCCTCGGGAAAAAGGCGGAGCCAGTACACGACATCACT
TTCCCAGTTTACCCCGCGCCACCTTCTCTAGGCACCGGATCAATTGCCGACCCCTCCC
CCCAACTTCTCGGGGACTGTGGGCGATGTGCGCTCTGCCCTAGTTGCTTGTGATTTCT
TTTCTTTTTTATTTTATTTCCATTATTTGAAATGTATTTGTTGTAGCAATGCTTTTGAC
ACGAAATAAATAAAAGAAAAAAAAAAAAAAAAAAAAAAAAAAAAAAAAAAAAAA
AAAAAAAAAAAAAAAAAAAAAAAAAAAAAAAAAAAAAAAAAA

Briefly, the template RNA for use with the Vingi-1_Acar gene modifying system comprises the following sequences from 5′ to 3′: 5′UTR, GFP, EF1alpha, 3′UTR and was co-delivered with the driver RNA.

The second gene modifying system used in this Example comprises a CR-1-1_PH driver encoded by a nucleic acid having a sequence of:

(SEQ ID NO: 905)
atgctgtataccatcttcctcgtttatgggatcctgtgttttttctttgtgtatttctgcatctacctgtacatcactgtttatctttgttttctgttc
tttctgtttctgcttctgtgcggtgacgtggaaagcaatcctggaccaggcagagcccgtggatgtcggttgctgtattgcaacatccgcggtctgcat
gctaatctggccgagctggactttgttagtcgcggggtggacgtggtctgctgttccgagactctggtggctgggcgtcgccacgatgccgag
ttggccctcgctggcttccagtccccctttcgtaggctctgtggctcaggccctggcttccgtggcatggcggtgtacgtcaggtctgggttct
gtgcctaccggcagagtgtgcacgaatgtgcctgccacgagattatcgtcgttcgcgtgtgtggacgtctgaacaattactatctgttctctctc
taccgctccccggccaccgatgactccctttacgattgccttctgactgccatggccagcatccagtccactgatccgaaggcagcettegtc
ttcgttggcgacgtaaacgcacatcaccgggactggctgggaagcgcctctccaacagattgtcacggcgtcgccgctctggacttttgcac
cctttccagctgcgtgcaactcgtccgtgggtccactcatatcgctggaaactgtctggatctggtgatgaccgacgtgcctgacctgatgac
cgtgaccgtcggatccccgattggaagtagcgaccactcccacctggtggtctcactggatctcaaccaggtcgtgcctgttgtggatacac
gcagggttgtgttcgtgAAAagccgcgccaactgggtcgccatcactcgcgctgtgaggactctgccttggcggcagattatccactcc
gaggaccctgtaagtgagctcaatgacctgaccgtgtctattctggaacgctttgttcccaagcgcaccatcctcgtgagaagccgcgataa
gccttggttcgatgaccagtgtagactggctttcgaggctaagcaggcggcttaccgtgcttggcgtaggagtcgggaccgcacgttgtgg
cagacctatgtggacaggcgggccgaggccaagcgcgtgtatgaagaggctcagaggcgccttcgccagaggagccgtgaaagcctg
ctcagtattgaccaccctcatcgctggtggtcagagctcaagggctccgtgtteggegccgaacccagcctgccccctctcgttggtcctgg
cggtggaatcattaccgatcccctcgccagggccgagctgctctccgcacattttgatggaaagcagtcacgegatgtcategccttgcccc
acggttgccacccggagccccgcctcacatctctggccttcaggtccggcgctatcaaggtcctcctggaaggactcgacccctacggtgg
cgttgaccccgtcggcatgtttcctctgttttacaagcagctggcggacgtcctggccccaaagttggccgttatctttcgtcggctgatccgc
ctcggcaactttccccgctgctggcgcactggtaatatcactcccatccccaagggccccgtgagtccttacgtggccaattaccgtcccatc
accttgacacctatcctgtctaaggttttcgaaaagctgatcgcaggcaagctgggtcgctttgctgaagtcactggcttgctccctgctggtc
agtttgcttatcgcatcggtctgggctgctgtgacgctctcctgtccgtgagccatcacctgcagtccgccctggactgccgctccgaagccc
gcctggtgcagctcgatttctccgcagcgtttgaccgcgttaatcacaggggtcttctgtataaattggagtccttcggcgtcgggggccggg
tgctgtcaattatccgtgatttcgtatccgagcgcacccagtctgtatccgtggacggtgttctgtccgccagcgtcggcgtggttagcggagt
cccccagggtagcgtgctgggacctctgctctttgtattgtatacgtccgacatgttctctagcctggagaataccctcattaactatgcagatg
actccactttgatggcagttattccggccccacgcctgcgtgacgccgtagcgcagtccctgaatagggacctgtcacgcatcagtgcctgg
tgttcagcttggtccatgaagctgaacgcgtccaagaccaaaagcatgattatctcaaggtccaggactttggtgcctcaacacccccagctg
gagatcgacgatactttgctgcaggagtcctcatccctggaaatcctgggcgtcgtgttcgatgagaagctcaccttcgagcctcacatcag
gcggctggtgagccgcgcgtctaccaagatcgggctgctccgcaaggtgaactccgtgttcggcgattcccaggtcgcgaggcgttgcttt
tacgctttcctgctccctgtgctggagtactgctctccggtatgggcgagtgccgcggatacccatcttcgtcttctggaccgccttgtctccag
cgccagccgtttgtgtgctgacaacgatctggtgaacctgagtcaccgtcggcgcgtggctgagctgtgtatgttctacaaggtgtacaataa
cgagaggcattggctgtactcatccctgccagccctgaaggtcttcgggcgcgaaacgcgtgccgcgtgcggtgctcactcttttacgttgg
aggcggttcgctgtcgcaccaaccagttctgccgctgctttgtgccttggtctgcgaaggtgtggaacctgctccctgcctccgctttcggga
gaatcggtctgcaggcatttaaaagcgctgtgaacggtttcctgttggactttctg

The CR-1 driver encoded by the nucleic acid has an amino acid sequence of:

(SEQ ID NO: 406)
MLYTIFLVYGILCFFFVYFCIYLYITVYLCFLFFLFLLLCGDVESNPGPGRARGCRLLYCNI
RGLHANLAELDFVSRGVDVVCCSETLVAGRRHDAELALAGFQSPFRRLCGSGPGFRGM
AVYVRSGFCAYRQSVHECACHEIIVVRVCGRLNNYYLFSLYRSPATDDSLYDCLLTAM
ASIQSTDPKAAFVFVGDVNAHHRDWLGSASPTDCHGVAALDFCTLSSCVQLVRGSTHIA
GNCLDLVMTDVPDLMTVTVGSPIGSSDHSHLVVSLDLNQVVPVVDTRRVVFVKSRAN
WVAITRAVRTLPWRQIIHSEDPVSELNDLTVSILERFVPKRTILVRSRDKPWFDDQCRLA
FEAKQAAYRAWRRSRDRTLWQTYVDRRAEAKRVYEEAQRRLRQRSRESLLSIDHPHR
WWSELKGSVFGAEPSLPPLVGPGGGIITDPLARAELLSAHFDGKQSRDVIALPHGCHPEP
RLTSLAFRSGAIKVLLEGLDPYGGVDPVGMFPLFYKQLADVLAPKLAVIFRRLIRLGNFP
RCWRTGNITPIPKGPVSPYVANYRPITLTPILSKVFEKLIAGKLGRFAEVTGLLPAGQFAY
RIGLGCCDALLSVSHHLQSALDCRSEARLVQLDFSAAFDRVNHRGLLYKLESFGVGGRV
LSIIRDFVSERTQSVSVDGVLSASVGVVSGVPQGSVLGPLLFVLYTSDMFSSLENTLINYA
DDSTLMAVIPAPRLRDAVAQSLNRDLSRISAWCSAWSMKLNASKTKSMIISRSRTLVPQ
HPQLEIDDTLLQESSSLEILGVVFDEKLTFEPHIRRLVSRASTKIGLLRKVNSVFGDSQVA
RRCFYAFLLPVLEYCSPVWASAADTHLRLLDRLVSSASRLCADNDLVNLSHRRRVAELC
MFYKVYNNERHWLYSSLPALKVFGRETRAACGAHSFTLEAVRCRTNQFCRCFVPWSA
KVWNLLPASAFGRIGLQAFKSAVNGFLLDFL

The CR-1 gene modifying system further comprises a plasmid that produces a template RNA having a sequence of:

(SEQ ID NO: 1005)
TCTATGGCTTCTGAGGCGGAAAGAACCAGCTGGGGCTCTAGGGGGTATCCCCCTGTG
GAATGTGTGTCAGTTAGGGTGTGGAAAGTCCCCAGGCTCCCCAGCAGGCAGAAGTA
TGCAAAGCATGCATCTCAATTAGTCAGCAACCAGGTGTGGAAAGTCCCCAGGCTCC
CCAGCAGGCAGAAGTATGCAAAGCATGCATCTCAATTAGTCAGCAACCATAGTCCC
GCCCCTAACTCCGCCCATCCCGCCCCTAACTCCGCCCAGTTCCGCCCATTCTCCGCCC
CATGGCTGACTAATTTTTTTTATTTATGCAGAGGCCGAGGCCGCCTCTGCCTCTGAGC
TATTCCAGAAGTAGTGAGGAGGCTTTTTTGGAGGCCTAGGCTTTTGCAAAAAGCTCC
CGGGAGCTTGTATATCCATTTTCGGATCTGATCAAGAGACAGGATGAGGATCGTTTC
GCATGACCGAGTACAAGCCCACGGTGCGCCTCGCCACCCGCGACGACGTCCCCAGG
GCCGTACGCACCCTCGCCGCCGCGTTCGCCGACTACCCCGCCACGCGCCACACCGTC
GATCCGGACCGCCACATCGAGCGGGTCACCGAGCTGCAAGAACTCTTCCTCACGCG
CGTCGGGCTCGACATCGGCAAGGTGTGGGTCGCGGACGACGGCGCCGCGGTGGCGG
TCTGGACCACGCCGGAGAGCGTCGAAGCGGGGGCGGTGTTCGCCGAGATCGGCCCG
CGCATGGCCGAGTTGAGCGGTTCCCGGCTGGCCGCGCAGCAACAGATGGAGGGCCT
CCTGGCGCCGCACCGGCCCAAGGAGCCCGCGTGGTTCCTGGCCACCGTCGGCGTCTC
GCCCGACCACCAGGGCAAGGGTCTGGGCAGCGCCGTCGTGCTCCCCGGAGTGGAGG
CCGCCGAGCGCGCCGGGGTGCCCGCCTTCCTGGAGACCTCCGCGCCCCGCAACCTCC
CCTTCTACGAGCGGCTCGGCTTCACCGTCACCGCCGACGTCGAGGTGCCCGAAGGAC
CGCGCACCTGGTGCATGACCCGCAAGCCCGGTGCCTAGGCGGGACTCTGGGGTTCG
AAATGACCGACCAAGCGACGCCCAACCTGCCATCACGAGATTTCGATTCCACCGCC
GCCTTCTATGAAAGGTTGGGCTTCGGAATCGTTTTCCGGGACGCCGGCTGGATGATC
CTCCAGCGCGGGGATCTCATGCTGGAGTTCTTCGCCCACCCCAACTTGTTTATTGCA
GCTTATAATGGTTACAAATAAAGCAATAGCATCACAAATTTCACAAATAAAGCATTT
TTTTCACTGCATTCTAGTTGTGGTTTGTCCAAACTCATCAATGTATCTTATCATGTCT
GTATACCGTCGACCTCTAGCTAGAGCTTGGCGTAATCATGGTCATAGCTGTTTCCTGT
GTGAAATTGTTATCCGCTCACAATTCCACACAACATACGAGCCGGAAGCATAAAGT
GTAAAGCCTGGGGTGCCTAATGAGTGAGCTAACTCACATTAATTGCGTTGCGCTCAC
TGCCCGCTTTCCAGTCGGGAAACCTGTCGTGCCAGCTGCATTAATGAATCGGCCAAC
GCGCGGGGAGAGGCGGTTTGCGTATTGGGCGCTCTTCCGCTTCCTCGCTCACTGACT
CGCTGCGCTCGGTCGTTCGGCTGCGGCGAGCGGTATCAGCTCACTCAAAGGCGGTA
ATACGGTTATCCACAGAATCAGGGGATAACGCAGGAAAGAACATGTGAGCAAAAG
GCCAGCAAAAGGCCAGGAACCGTAAAAAGGCCGCGTTGCTGGCGTTTTTCCATAGG
CTCCGCCCCCCTGACGAGCATCACAAAAATCGACGCTCAAGTCAGAGGTGGCGAAA
CCCGACAGGACTATAAAGATACCAGGCGTTTCCCCCTGGAAGCTCCCTCGTGCGCTC
TCCTGTTCCGACCCTGCCGCTTACCGGATACCTGTCCGCCTTTCTCCCTTCGGGAAGC
GTGGCGCTTTCTCATAGCTCACGCTGTAGGTATCTCAGTTCGGTGTAGGTCGTTCGCT
CCAAGCTGGGCTGTGTGCACGAACCCCCCGTTCAGCCCGACCGCTGCGCCTTATCCG
GTAACTATCGTCTTGAGTCCAACCCGGTAAGACACGACTTATCGCCACTGGCAGCAG
CCACTGGTAACAGGATTAGCAGAGCGAGGTATGTAGGCGGTGCTACAGAGTTCTTG
AAGTGGTGGCCTAACTACGGCTACACTAGAAGAACAGTATTTGGTATCTGCGCTCTG
CTGAAGCCAGTTACCTTCGGAAAAAGAGTTGGTAGCTCTTGATCCGGCAAACAAAC
CACCGCTGGTAGCGGTTTTTTTGTTTGCAAGCAGCAGATTACGCGCAGAAAAAAAGG
ATCTCAAGAAGATCCTTTGATCTTTTCTACGGGGTCTGACGCTCAGTGGAACGAAAA
CTCACGTTAAGGGATTTTGGTCATGAGATTATCAAAAAGGATCTTCACCTAGATCCT
TTTAAATTAAAAATGAAGTTTTAAATCAATCTAAAGTATATATGAGTAAACTTGGTC
TGACAGTTACCAATGCTTAATCAGTGAGGCACCTATCTCAGCGATCTGTCTATTTCGT
TCATCCATAGTTGCCTGACTCCCCGTCGTGTAGATAACTACGATACGGGAGGGCTTA
CCATCTGGCCCCAGTGCTGCAATGATACCGCGAGACCCACGCTCACCGGCTCCAGAT
TTATCAGCAATAAACCAGCCAGCCGGAAGGGCCGAGCGCAGAAGTGGTCCTGCAAC
TTTATCCGCCTCCATCCAGTCTATTAATTGTTGCCGGGAAGCTAGAGTAAGTAGTTC
GCCAGTTAATAGTTTGCGCAACGTTGTTGCCATTGCTACAGGCATCGTGGTGTCACG
CTCGTCGTTTGGTATGGCTTCATTCAGCTCCGGTTCCCAACGATCAAGGCGAGTTAC
ATGATCCCCCATGTTGTGCAAAAAAGCGGTTAGCTCCTTCGGTCCTCCGATCGTTGT
CAGAAGTAAGTTGGCCGCAGTGTTATCACTCATGGTTATGGCAGCACTGCATAATTC
TCTTACTGTCATGCCATCCGTAAGATGCTTTTCTGTGACTGGTGAGTACTCAACCAAG
TCATTCTGAGAATAGTGTATGCGGCGACCGAGTTGCTCTTGCCCGGCGTCAATACGG
GATAATACCGCGCCACATAGCAGAACTTTAAAAGTGCTCATCATTGGAAAACGTTCT
TCGGGGCGAAAACTCTCAAGGATCTTACCGCTGTTGAGATCCAGTTCGATGTAACCC
ACTCGTGCACCCAACTGATCTTCAGCATCTTTTACTTTCACCAGCGTTTCTGGGTGAG
CAAAAACAGGAAGGCAAAATGCCGCAAAAAAGGGAATAAGGGCGACACGGAAATG
TTGAATACTCATACTCTTCCTTTTTCAATATTATTGAAGCATTTATCAGGGTTATTGT
CTCATGAGCGGATACATATTTGAATGTATTTAGAAAAATAAACAAATAGGGGTTCCG
CGCACATTTCCCCGAAAAGTGCCACCTGACGTCGACGGATCGGGAGATCTCCCGATC
CCCTATGGTGCACTCTCAGTACAATCTGCTCTGATGCCGCATAGTTAAGCCAGTATC
TGCTCCCTGCTTGTGTGTTGGAGGTCGCTGAGTAGTGCGCGAGCAAAATTTAAGCTA
CAACAAGGCAAGGCTTGACCGACAATTGCATGAAGAATCTGCTTAGGGTTAGGCGT
TTTGCGCTGCTTCGCGATGTACGGGCCAGATATACGCgttgacattgattattgactagttattaatagtaat
caattacggggtcattagttcatagcccatatatggagttccgcgttacataacttacggtaaatggcccgcctggctgaccgcccaacgacc
cccgcccattgacgtcaataatgacgtatgttcccatagtaacgccaatagggactttccattgacgtcaatgggtggagtatttacggtaaac
tgcccacttggcagtacatcaagtgtatcatatgccaagtacgccccctattgacgtcaatgacggtaaatggcccgcctggcattatgccca
gtacatgaccttatgggactttcctacttggcagtacatctacgtattagtcatcgctattaccatggtgatgcggttttggcagtacatcaatgg
gcgtggatagcggtttgactcacggggatttccaagtctccaccccattgacgtcaatgggagtttgttttggcaccaaaatcaacgggacttt
ccaaaatgtcgtaacaactccgccccattgacgcaaatgggcggtaggcgtgtacggtgggaggtctatataagcagagctctctggctaa
ctagagaacccactgcttactggcttatcgaaattaatacgactcactatagggagacccaagctggcgtggcctgcgcgttatcaggccgc
ccccattgtccggccaccggacgcctctttgtttgtatgagctggctttgggtttggggctctgggtagcccctgagttggaggaaatgccact
ctaaaggcggatgagacctcttttggtccaacggcatccctgagaggagatgaacaggctggtgccatagctgggtactcagtcttccgca
gtctgctcctctctctcttccatcaggtagtaccacctgatgctcttttcctttcctttctcttcttcttcttttcggccaaagcctgtactggtgatggc
agggtctggggcgcgtagatagctggccgctttcgtagctatcgttactgtcgatttgtttttgctttattGTTTACGGATCCGAAT
TCTCGACGGATCGATCCGAACAAACGACCCAACACCCGTGCGTTTTATTCTGTCTTTT
TATTGCCGATCCCCCGGCCGCTTTACTTGTACAGCTCGTCCATGCCGAGAGTGATCC
CGGCGGCGGTCACGAACTCCAGCAGGACCATGTGATCGCGCTTCTCGTTGGGGTCTT
TGCTCAGGGCGGACTGGGTGCTCAGGTAGTGGTTGTCGGGCAGCAGCACGGGGCCG
TCGCCGATGGGGGTGTTCTGCTGGTAGTGGTCGGCGAGCTGCACGCTGCCGTCCTCG
ATGTTGTGGCGGATCTTGAAGTTCACCTTGATGCCGTTCTTCTGCTTGTCGGCCATGA
TATAGACGTTGTGGCTGTTGTAGTTGTACTCCAGCTTGTGCCCCAGGATGTTGCCGTC
CTCCTTGAAGTCGATGCCCTTCAGCTCGATGCGGTTCACCAGGGTGTCGCCCTCGAA
CTTCACCTCGGCGCGGGTCTTGTAGTTGCCGTCGTCCTTGAAGAAGATGGTGCGCTC
CTGGACGTAGCCTTCGGGCATGGCGGACTTGAAGAAGTCGTGCTGCTTCATGTGGTC
GGGGTAGCGGCTGAAGCACTGCACGCCGTAGGTCAGGGTGGTCACGAGGGTGGGCC
AGGGCACGGGCAGCTTGCCGGTGGTGCAGATGAACTTCAGGGTCAGCTTGCCGTAG
GTGGCATCGCCCTCGCCCTCGCCGGACACGCTGAACTTGTGGCCGTTTACGTCGCCG
TCCAGCTCGACCAGGATGGGCACCACCCCGGTGAACAGCTCCTCGCCCTTGCTCACC
ATGGTGGCTTTACCAACAGTACCGGAATGCCAAGCTTGGGTCctgtgttctggeggcaaacccgtt
gcgaaaaagaacgttcacggcgactactgcacttatatacggttctcccccaccctcgggaaaaaggcggagccagtacacgacatcactt
tcccagtttacccegcgccaccttctctaggcaceggatcaattgccgacccctccccccaacttcteggggactgtgggcgatgtgcgctct
gccctagcagcgttttgggcttcccggtgttgcaagatgcttcatttgggttgtattacctgtccgtgggagctcaagcgtgaagttttaataata
ataataaCCGCTGATCAGCCTCGACTGTGCCTTCTAGTTGCCAGCCATCTGTTGTTTGCCC
CTCCCCCGTGCCTTCCTTGACCCTGGAAGGTGCCACTCCCACTGTCCTTTCCTAATAA
AATGAGGAAATTGCATCGCATTGTCTGAGTAGGTGTCATTCTATTCTGGGGGGTGGG
GTGGGGCAGGACAGCAAGGGGGAGGATTGGGAAGACAATAGCAGGCATGCTGGGG
ATGCGGTGGGC

The gene modifying system was introduced as RNA into iPSC using Neon transfection system.

As a comparison, integration profile data for lentiviral gene delivery system was obtained from the following publication, Sherrill-Mix et al. 2013, Retrovirology.

Integration profile was assessed using S-EPTS/LM-PCR (shearing extension primer tag selection ligation-mediated PCR) (Schmidt et al., 2001) and analyzed using the GENE-IS tool suite (Afzal, Wilkening, et al., 2017). All insertion sites were located on the human genome assembly GRCh38 and were categorized in relation to gene annotations found in Gencode (gencodegenes.org) version 39. Site were classified as exonic and intronic if located within region boundaries, upstream of TSS if located within 2 kb upstream of annotated transcription start site, and intergenic if found in any region not annotated as a protein coding gene. See FIGS. 2A-2B.

The analysis showed the following proportions of insertion sites from the Vingi-1_Acar, CR-1-1_PH, and lentivirus systems:

| Insertion site | Vingi-1_Acar | CR-1-1_PH | Lentirvirus |
|---|---|---|---|
| Exonic | 5% | 4% | 6% |
| Intronic | 47% | 46% | 72% |
| Upstream of TSS | 6% | 6% | 11% |
| Intergenic | 43% | 45% | 17% |

Overall, this analysis showed that the gene modifying systems tested demonstrated a higher level of insertions outside of genes compared to a lentiviral system. Without wishing to be bound by theory, the integration pattern of the gene modifying systems is advantageous because, in some embodiments, it is desired to reduce disrupting expression of endogenous genes in the host cell.

### Example 2: Retrotransposon-Based Gene Modifying Systems do not Activate DNA Damage Response Pathways in Primary Cells

This example demonstrates that editing of a genome using a gene modifying system described herein does not activate the DNA damage response in primary cells.

The gene modifying systems used in this Example comprise a nucleic acid encoding a Vingi-1_Acar, CR1-1_PH, RTE-1_MD, and RTE-3_BF drivers as described herein. For Vingi-1_Acar, the gene modifying system comprises a nucleic acid sequence of:

(SEQ ID NO: 900)
ATGGACGAGTACCAGCGCTCCTTGTCCCGCCCCCTGTTGACAATCATGAGTATTAAT
ATCGAAGGCTTGAGCCTCGCGAAAGAGGAACTGCTTGCCAAGATGTCCGAGGACAT
CAGCTGCGACATTCTGTGCATCCAAGAGACCCATCGCGACATCACCATGCGTCGCCC
CAAGATCCTCGGTATGCAGCTTGCGGTGGAGCGCCCTCACCGCCAGTACGGCTCCGC
GATTTTCGTGCGTTCCGGTGTGGCCATTTCTGCTACCAGCCTCACCGAGGTTAACAAT
ATTGAGATCCTGTCAGTGGAGCTCGATTCCTGTACCGTCTCCAGTCTGTACAAACCT
CCCGGTGCAGACTTCTACTTTACTCCCCCGACGTCTTGCCACAACCATGAGGCTCAT
TTTGTGGTTGGTGATTTCAATTCCCACTCCTGCGTGTGGGGCTATGACGAGGATGAC
CGCAACGGCGAAGCTGTTCTGACCTGGGCCGATAATTCCCGCATGTCCCTGCTCCAC
GATAGTAAGCTGCCTCCCTCCTTCAACAGTGGGAGATGGAAGCGCGGGTACAACCC
TGATCTTATCTTCGTCAAAGAATCCATCTCTCATCAGTGTACCAAGCGCGTCCTCAAC
CCGATTCCTAACACTCAGCACCGCCCCATTTGTTGCGTAGCATATGCTGCCGTGCGT
CCCAAGTCTGTCCCCTTCAGAAGGAGATACAACTTCAACAAGGCTAACTGGACCAA
GTTTACCGAAACCTTGGAAGCGGCAATCTCCGACATTGAGCCATCTATCGAGAATTA
TGATCTCTTCGTTGAGGCAGTCAAGCGGTCCTCTCGCCTGAGTATCCCCCGTGGATG
TAGGACCTCCTACCTGCCCGGCCTGAACGAAGAGTCACTCAATCAGCTGCAGGAAT
ACCTGCGCCTGTTCCAAGAGAATCCTTACTCCGATGGCACCATCGCTGCCGGACAAA
AACTGAGCACCGCTCTGGCGAATGCTAAGAAAGACCGCTGGATCGAGCTGCTCGAG
AACCTGGACATGTCCAAGTCTAGCCGGAAAGCTTGGCAGCTCCTGCGCCGGCTGGA
CAGCGACCCCCTCGTGAATCCTGGCCATGCCAATGTAACGCCGGACCAGATCGCCC
ATCAACTGATCCAGAACGGCAAGACCAATTGCAGTAGAATCAAGATGAAGATCAAC
AGAGTACCCGAGTTGGAAACACACCAGCTGTCTAGTCCTCTCAACCTCAAAGAGCTC
CGCGAAGCAATCAAAAGGTGTAAGACGGGCAAAGCTCCCGGTCTGGATGACCTGAT
GATGGAACAGATCAAACACTTGGGGGCCAAGGCTGAGAACTGGTTGCTGAAATTCT
ACAACCAGTGCCTGGCTCACAAGCAGATCCCCCGTGCGTGGCGCAAGACTAAGATT
ATCGCCATCTTGAAGCCGGGCAAGGATGCTAGTAACGCCCGCAACTACCGTCCTATT
TCCCTCCTGTGTCACCTTTACAAGGTGTACGAGCGCATGCTCCTGAACCGCTTGGGC
CCCGTCATCGAGCCTAAACTGATCGCCCAACAGGCTGGTTTCCGCCCAGGTAAGAAC
TGCACTGGACAGATCCTGCACCTTACGGAGCACATCGAGGAAGGTTACGAGAAGGG
CTGCATCACCGGAACCGTCTTCGTGGACCTGACTGCGGCCTACGACACGGTTCAGCA
CAGAAAAATGCTCCACAAGGTATATCATATTACCCGGGATTTCGACTTCACCAAAAC
AGTGCAGACGCTTCTGGAGAATCGCTCCTTTTACGTCGAGTTCCAGGGTCAGAAAAG
TCGGTGGAGGCGCCAGAAGAACGGCTTGCCCCAGGGTAGCGTGCTCGCCCCAACCC
TGTTTAATATCTTCACCAACGACCAGCCACAGCCCCCGCTGACAAAATCCTTTATCT
ATGCCGACGATCTTGGCCTGACCACGCAAGCCAAGGACTTTGAGACAGTCGAGAAA
CAGCTCACAAACGCACTCAAGGACCTGAGTTCCTATTACAAGGAGAACCATCTCAA
ACCTAACCCCGCTAAGACACAGGTCTGCGCTTTTCACCTGAGGAATCGGGAGGCCA
ACCGTAAACTGAAGGTGACATGGGAGGGACAGGAACTGGAGCACTGTTTCCACCCC
AAGTATTTGGGCGTGACTCTCGATCGCACCCTTACGTATCGGAAGCACTGCATGAAC
ACTAAGCATAAGGTGGCGGCCAGAAATAACATCCTGCGTAAACTCACAGGAAGCGC
CTGGGGCGCCGACCCACAGGTCATCCGTACCAGTGCTCTGGCCCTGTCTTTCTCCAC
TGCTGAATATGCGTGTCCGGTGTGGCACAAGTCCGCCCACGCGAAGCAGGTGGATA
TTGCCCTGAACGAGACATGCCGGATTATCACTGGTTGCCTCAAGCCTACCCCCGTCG
ACAAGCTCTATAAGTTGGCCGGCATTGCACCCCCTGATGTCCGTCGCGAAGTCGCAG
CCAATGGCGAGCGGAAAAAGGTAGAACACTGCGAATCACATCCGCTGCATGGATAT
CATCCCCCTCCCACCCGGCTTAAGAGCCGTAAGGGCTTCATGCGCACAACCACTCCG
CTGGATGTGCCTCCCGCTGCCGCGCGCGTGAGTCTGTGGGCCGCTAAGCCCGGTAAT
TCCAATTGGATGGCACCGCAAGAGGGCCTCCCGCCCGGAGCCAATCAAGAGTGGGC
CACATGGAAGTCCTTGAACAGGCTGCGCTCCGGTGTGGGACGCTCTAAGGACAACC
TGGCCCGTTGGCACTATCTGGAAGAGTCATCCACCCTGTGCGACTGTGGTGCTGAAC
AGACTACCCAGCACATGTACGCTTGCCCCCAATGCCCTGCCTCCTGCACGGAAGAGG
AACTCTTTAAGGCAACCGATAACGCAGTGGCAGTGGCGCGCTTTTGGAGTAAGACA
ATC

The Vingi-1_Acar driver encoded by the nucleic acid has an amino acid sequence of:

(SEQ ID NO: 405)
MDEYQRSLSRPLLTIMSINIEGLSLAKEELLAKMSEDISCDILCIQETHRDITMRRPKILGM
QLAVERPHRQYGSAIFVRSGVAISATSLTEVNNIEILSVELDSCTVSSLYKPPGADFYFTPP
TSCHNHEAHFVVGDFNSHSCVWGYDEDDRNGEAVLTWADNSRMSLLHDSKLPPSFNS
GRWKRGYNPDLIFVKESISHQCTKRVLNPIPNTQHRPICCVAYAAVRPKSVPFRRRYNFN
KANWTKFTETLEAAISDIEPSIENYDLFVEAVKRSSRLSIPRGCRTSYLPGLNEESLNQLQ
EYLRLFQENPYSDGTIAAGQKLSTALANAKKDRWIELLENLDMSKSSRKAWQLLRRLD
SDPLVNPGHANVTPDQIAHQLIQNGKTNCSRIKMKINRVPELETHQLSSPLNLKELREAIK
RCKTGKAPGLDDLMMEQIKHLGAKAENWLLKFYNQCLAHKQIPRAWRKTKIIAILKPG
KDASNARNYRPISLLCHLYKVYERMLLNRLGPVIEPKLIAQQAGFRPGKNCTGQILHLTE
HIEEGYEKGCITGTVFVDLTAAYDTVQHRKMLHKVYHITRDFDFTKTVQTLLENRSFYV
EFQGQKSRWRRQKNGLPQGSVLAPTLFNIFTNDQPQPPLTKSFIYADDLGLTTQAKDFET
VEKQLTNALKDLSSYYKENHLKPNPAKTQVCAFHLRNREANRKLKVTWEGQELEHCF
HPKYLGVTLDRTLTYRKHCMNTKHKVAARNNILRKLTGSAWGADPQVIRTSALALSFS
TAEYACPVWHKSAHAKQVDIALNETCRIITGCLKPTPVDKLYKLAGIAPPDVRREVAAN
GERKKVEHCESHPLHGYHPPPTRLKSRKGFMRTTTPLDVPPAAARVSLWAAKPGNSNW
MAPQEGLPPGANQEWATWKSLNRLRSGVGRSKDNLARWHYLEESSTLCDCGAEQTTQ
HMYACPQCPASCTEEELFKATDNAVAVARFWSKTI

The Vingi-1_Acar gene modifying system further comprises a template RNA having a sequence of:

(SEQ ID NO: 1000)
AGGGGGACACGGAAAGAGCCTCCCCGAAGATTGAGTGAATTCAGTCGGGCGTCCCC
TGGGCAACGTTTCTTGTAAGCGGCCGATCTTTCCACCCCAAAAGCATTGGATGAGTT
TACGGATCCGAATTCTCGACGGATCGATCCGAACAAACGACCCAACACCCGTGCGT
TTTATTCTGTCTTTTTATTGCCGATCCCCCGGCCGCTTTACTTGTACAGCTCGTCCATG
CCGAGAGTGATCCCGGCGGCGGTCACGAACTCCAGCAGGACCATGTGATCGCGCTT
CTCGTTGGGGTCTTTGCTCAGGGCGGACTGGGTGCTCAGGTAGTGGTTGTCGGGCAG
CAGCACGGGGCCGTCGCCGATGGGGGTGTTCTGCTGGTAGTGGTCGGCGAGCTGCA
CGCTGCCGTCCTCGATGTTGTGGCGGATCTTGAAGTTCACCTTGATGCCGTTCTTCTG
CTTGTCGGCCATGATATAGACGTTGTGGCTGTTGTAGTTGTACTCCAGCTTGTGCCCC
AGGATGTTGCCGTCCTCCTTGAAGTCGATGCCCTTCAGCTCGATGCGGTTCACCAGG
GTGTCGCCCTCGAACTTCACCTCGGCGCGGGTCTTGTAGTTGCCGTCGTCCTTGAAG
AAGATGGTGCGCTCCTGGACGTAGCCTTCGGGCATGGCGGACTTGAAGAAGTCGTG
CTGCTTCATGTGGTCGGGGTAGCGGCTGAAGCACTGCACGCCGTAGGTCAGGGTGGT
CACGAGGGTGGGCCAGGGCACGGGCAGCTTGCCGGTGGTGCAGATGAACTTCAGGG
TCAGCTTGCCGTAGGTGGCATCGCCCTCGCCCTCGCCGGACACGCTGAACTTGTGGC
CGTTTACGTCGCCGTCCAGCTCGACCAGGATGGGCACCACCCCGGTGAACAGCTCCT
CGCCCTTGCTCACCATGGTGGCTTTACCAACAGTACCGGAATGCCAAGCTTGGGTCC
TGTGTTCTGGCGGCAAACCCGTTGCGAAAAAGAACGTTCACGGCGACTACTGCACTT
ATATACGGTTCTCCCCCACCCTCGGGAAAAAGGCGGAGCCAGTACACGACATCACT
TTCCCAGTTTACCCCGCGCCACCTTCTCTAGGCACCGGATCAATTGCCGACCCCTCCC
CCCAACTTCTCGGGGACTGTGGGCGATGTGCGCTCTGCCCTAGTTGCTTGTGATTTCT
TTTCTTTTTTATTTTATTTCCATTATTTGAAATGTATTTGTTGTAGCAATGCTTTTGAC
ACGAAATAAATAAAAGAAAAAAAAAAAAAAAAAAAAAAAAAAAAAAAAAAAAAA
AAAAAAAAAAAAAAAAAAAAAAAAAAAAAAAAAAAAAAAAAA

Briefly, the template RNA for use with the Vingi-1_Acar, CR1-1_PH, RTE-1_MD, and RTE-3_BF gene modifying systems comprises the following sequences from 5′ to 3′: 5′UTR, GFP, EF1alpha, 3′UTR and was co-delivered with the driver RNA. An inactive template RNA without UTR sequences was co-delivered with the driver RNA as a negative control. Vingi-1_Acar template RNA alone or driver RNA alone were also used as negative controls. The topoisomerase II inhibitor etoposide was added to the cells to promote DNA damage and was used as a positive control.

The gene modifying systems and any control constructs were introduced as RNAs into either iPSC or activated T cells using Neon transfection system or Amaxa 4D-Nucleofector system, respectively.

Induction of the DNA damage response was assessed using the following methodology. Briefly, antibodies directed to phosphorylated p53, p21, and Vinculin were used for western blot on cell lysates from iPSC or T cells transfected 24 hours earlier with the RNAs of the gene modifying system or appropriate controls. Phosphorylated p53 and p21 are known markers of DNA damage while vinculin is a commonly used control for western blot analysis.

As shown in FIG. 3A, Vingi-1_Acar, CR1-1_PH, RTE-1_MD, and RTE-3_BF gene modifying systems drive integration and expression of a template in iPSC or T cells when delivered as all-RNA compositions.

As shown in FIG. 3B, introduction and editing using a gene modifying system did not activate the DNA damage response in T cells and iPSCs. In contrast, introduction of etoposide activated DNA damage response, as shown by the increased western blot signal using anti-phosphorylated p53 and anti-p21 antibodies.

### Example 3: Retrotransposon-Based Gene Modifying Systems do not Activate Interferon Response in Primary Cells

This example demonstrates that cell editing using a gene modifying system described herein does not activate interferon response in primary cells.

The gene modifying system used in this Example comprises a nucleic acid encoding a Vingi-1_Acar driver having a nucleic acid sequence of:

(SEQ ID NO: 900)
ATGGACGAGTACCAGCGCTCCTTGTCCCGCCCCCTGTTGACAATCATGAGTATTAAT
ATCGAAGGCTTGAGCCTCGCGAAAGAGGAACTGCTTGCCAAGATGTCCGAGGACAT
CAGCTGCGACATTCTGTGCATCCAAGAGACCCATCGCGACATCACCATGCGTCGCCC
CAAGATCCTCGGTATGCAGCTTGCGGTGGAGCGCCCTCACCGCCAGTACGGCTCCGC
GATTTTCGTGCGTTCCGGTGTGGCCATTTCTGCTACCAGCCTCACCGAGGTTAACAAT
ATTGAGATCCTGTCAGTGGAGCTCGATTCCTGTACCGTCTCCAGTCTGTACAAACCT
CCCGGTGCAGACTTCTACTTTACTCCCCCGACGTCTTGCCACAACCATGAGGCTCAT
TTTGTGGTTGGTGATTTCAATTCCCACTCCTGCGTGTGGGGCTATGACGAGGATGAC
CGCAACGGCGAAGCTGTTCTGACCTGGGCCGATAATTCCCGCATGTCCCTGCTCCAC
GATAGTAAGCTGCCTCCCTCCTTCAACAGTGGGAGATGGAAGCGCGGGTACAACCC
TGATCTTATCTTCGTCAAAGAATCCATCTCTCATCAGTGTACCAAGCGCGTCCTCAAC
CCGATTCCTAACACTCAGCACCGCCCCATTTGTTGCGTAGCATATGCTGCCGTGCGT
CCCAAGTCTGTCCCCTTCAGAAGGAGATACAACTTCAACAAGGCTAACTGGACCAA
GTTTACCGAAACCTTGGAAGCGGCAATCTCCGACATTGAGCCATCTATCGAGAATTA
TGATCTCTTCGTTGAGGCAGTCAAGCGGTCCTCTCGCCTGAGTATCCCCCGTGGATG
TAGGACCTCCTACCTGCCCGGCCTGAACGAAGAGTCACTCAATCAGCTGCAGGAAT
ACCTGCGCCTGTTCCAAGAGAATCCTTACTCCGATGGCACCATCGCTGCCGGACAAA
AACTGAGCACCGCTCTGGCGAATGCTAAGAAAGACCGCTGGATCGAGCTGCTCGAG
AACCTGGACATGTCCAAGTCTAGCCGGAAAGCTTGGCAGCTCCTGCGCCGGCTGGA
CAGCGACCCCCTCGTGAATCCTGGCCATGCCAATGTAACGCCGGACCAGATCGCCC
ATCAACTGATCCAGAACGGCAAGACCAATTGCAGTAGAATCAAGATGAAGATCAAC
AGAGTACCCGAGTTGGAAACACACCAGCTGTCTAGTCCTCTCAACCTCAAAGAGCTC
CGCGAAGCAATCAAAAGGTGTAAGACGGGCAAAGCTCCCGGTCTGGATGACCTGAT
GATGGAACAGATCAAACACTTGGGGGCCAAGGCTGAGAACTGGTTGCTGAAATTCT
ACAACCAGTGCCTGGCTCACAAGCAGATCCCCCGTGCGTGGCGCAAGACTAAGATT
ATCGCCATCTTGAAGCCGGGCAAGGATGCTAGTAACGCCCGCAACTACCGTCCTATT
TCCCTCCTGTGTCACCTTTACAAGGTGTACGAGCGCATGCTCCTGAACCGCTTGGGC
CCCGTCATCGAGCCTAAACTGATCGCCCAACAGGCTGGTTTCCGCCCAGGTAAGAAC
TGCACTGGACAGATCCTGCACCTTACGGAGCACATCGAGGAAGGTTACGAGAAGGG
CTGCATCACCGGAACCGTCTTCGTGGACCTGACTGCGGCCTACGACACGGTTCAGCA
CAGAAAAATGCTCCACAAGGTATATCATATTACCCGGGATTTCGACTTCACCAAAAC
AGTGCAGACGCTTCTGGAGAATCGCTCCTTTTACGTCGAGTTCCAGGGTCAGAAAAG
TCGGTGGAGGCGCCAGAAGAACGGCTTGCCCCAGGGTAGCGTGCTCGCCCCAACCC
TGTTTAATATCTTCACCAACGACCAGCCACAGCCCCCGCTGACAAAATCCTTTATCT
ATGCCGACGATCTTGGCCTGACCACGCAAGCCAAGGACTTTGAGACAGTCGAGAAA
CAGCTCACAAACGCACTCAAGGACCTGAGTTCCTATTACAAGGAGAACCATCTCAA
ACCTAACCCCGCTAAGACACAGGTCTGCGCTTTTCACCTGAGGAATCGGGAGGCCA
ACCGTAAACTGAAGGTGACATGGGAGGGACAGGAACTGGAGCACTGTTTCCACCCC
AAGTATTTGGGCGTGACTCTCGATCGCACCCTTACGTATCGGAAGCACTGCATGAAC
ACTAAGCATAAGGTGGCGGCCAGAAATAACATCCTGCGTAAACTCACAGGAAGCGC
CTGGGGCGCCGACCCACAGGTCATCCGTACCAGTGCTCTGGCCCTGTCTTTCTCCAC
TGCTGAATATGCGTGTCCGGTGTGGCACAAGTCCGCCCACGCGAAGCAGGTGGATA
TTGCCCTGAACGAGACATGCCGGATTATCACTGGTTGCCTCAAGCCTACCCCCGTCG
ACAAGCTCTATAAGTTGGCCGGCATTGCACCCCCTGATGTCCGTCGCGAAGTCGCAG
CCAATGGCGAGCGGAAAAAGGTAGAACACTGCGAATCACATCCGCTGCATGGATAT
CATCCCCCTCCCACCCGGCTTAAGAGCCGTAAGGGCTTCATGCGCACAACCACTCCG
CTGGATGTGCCTCCCGCTGCCGCGCGCGTGAGTCTGTGGGCCGCTAAGCCCGGTAAT
TCCAATTGGATGGCACCGCAAGAGGGCCTCCCGCCCGGAGCCAATCAAGAGTGGGC
CACATGGAAGTCCTTGAACAGGCTGCGCTCCGGTGTGGGACGCTCTAAGGACAACC
TGGCCCGTTGGCACTATCTGGAAGAGTCATCCACCCTGTGCGACTGTGGTGCTGAAC
AGACTACCCAGCACATGTACGCTTGCCCCCAATGCCCTGCCTCCTGCACGGAAGAGG
AACTCTTTAAGGCAACCGATAACGCAGTGGCAGTGGCGCGCTTTTGGAGTAAGACA
ATC

The Vingi-1_Acar driver encoded by the nucleic acid has an amino acid sequence of:

(SEQ ID NO: 405)
MDEYQRSLSRPLLTIMSINIEGLSLAKEELLAKMSEDISCDILCIQETHRDITMRRPKILGM
QLAVERPHRQYGSAIFVRSGVAISATSLTEVNNIEILSVELDSCTVSSLYKPPGADFYFTPP
TSCHNHEAHFVVGDFNSHSCVWGYDEDDRNGEAVLTWADNSRMSLLHDSKLPPSFNS
GRWKRGYNPDLIFVKESISHQCTKRVLNPIPNTQHRPICCVAYAAVRPKSVPFRRRYNFN
KANWTKFTETLEAAISDIEPSIENYDLFVEAVKRSSRLSIPRGCRTSYLPGLNEESLNQLQ
EYLRLFQENPYSDGTIAAGQKLSTALANAKKDRWIELLENLDMSKSSRKAWQLLRRLD
SDPLVNPGHANVTPDQIAHQLIQNGKTNCSRIKMKINRVPELETHQLSSPLNLKELREAIK
RCKTGKAPGLDDLMMEQIKHLGAKAENWLLKFYNQCLAHKQIPRAWRKTKIIAILKPG
KDASNARNYRPISLLCHLYKVYERMLLNRLGPVIEPKLIAQQAGFRPGKNCTGQILHLTE
HIEEGYEKGCITGTVFVDLTAAYDTVQHRKMLHKVYHITRDFDFTKTVQTLLENRSFYV
EFQGQKSRWRRQKNGLPQGSVLAPTLFNIFTNDQPQPPLTKSFIYADDLGLTTQAKDFET
VEKQLTNALKDLSSYYKENHLKPNPAKTQVCAFHLRNREANRKLKVTWEGQELEHCF
HPKYLGVTLDRTLTYRKHCMNTKHKVAARNNILRKLTGSAWGADPQVIRTSALALSFS
TAEYACPVWHKSAHAKQVDIALNETCRIITGCLKPTPVDKLYKLAGIAPPDVRREVAAN
GERKKVEHCESHPLHGYHPPPTRLKSRKGFMRTTTPLDVPPAAARVSLWAAKPGNSNW
MAPQEGLPPGANQEWATWKSLNRLRSGVGRSKDNLARWHYLEESSTLCDCGAEQTTQ
HMYACPQCPASCTEEELFKATDNAVAVARFWSKTI

The Vingi-1_Acar gene modifying system further comprises a template RNA having a sequence of:

(SEQ ID NO: 1000)
AGGGGGACACGGAAAGAGCCTCCCCGAAGATTGAGTGAATTCAGTCGGGCGTCCCC
TGGGCAACGTTTCTTGTAAGCGGCCGATCTTTCCACCCCAAAAGCATTGGATGAGTT
TACGGATCCGAATTCTCGACGGATCGATCCGAACAAACGACCCAACACCCGTGCGT
TTTATTCTGTCTTTTTATTGCCGATCCCCCGGCCGCTTTACTTGTACAGCTCGTCCATG
CCGAGAGTGATCCCGGCGGCGGTCACGAACTCCAGCAGGACCATGTGATCGCGCTT
CTCGTTGGGGTCTTTGCTCAGGGCGGACTGGGTGCTCAGGTAGTGGTTGTCGGGCAG
CAGCACGGGGCCGTCGCCGATGGGGGTGTTCTGCTGGTAGTGGTCGGCGAGCTGCA
CGCTGCCGTCCTCGATGTTGTGGCGGATCTTGAAGTTCACCTTGATGCCGTTCTTCTG
CTTGTCGGCCATGATATAGACGTTGTGGCTGTTGTAGTTGTACTCCAGCTTGTGCCCC
AGGATGTTGCCGTCCTCCTTGAAGTCGATGCCCTTCAGCTCGATGCGGTTCACCAGG
GTGTCGCCCTCGAACTTCACCTCGGCGCGGGTCTTGTAGTTGCCGTCGTCCTTGAAG
AAGATGGTGCGCTCCTGGACGTAGCCTTCGGGCATGGCGGACTTGAAGAAGTCGTG
CTGCTTCATGTGGTCGGGGTAGCGGCTGAAGCACTGCACGCCGTAGGTCAGGGTGGT
CACGAGGGTGGGCCAGGGCACGGGCAGCTTGCCGGTGGTGCAGATGAACTTCAGGG
TCAGCTTGCCGTAGGTGGCATCGCCCTCGCCCTCGCCGGACACGCTGAACTTGTGGC
CGTTTACGTCGCCGTCCAGCTCGACCAGGATGGGCACCACCCCGGTGAACAGCTCCT
CGCCCTTGCTCACCATGGTGGCTTTACCAACAGTACCGGAATGCCAAGCTTGGGTCC
TGTGTTCTGGCGGCAAACCCGTTGCGAAAAAGAACGTTCACGGCGACTACTGCACTT
ATATACGGTTCTCCCCCACCCTCGGGAAAAAGGCGGAGCCAGTACACGACATCACT
TTCCCAGTTTACCCCGCGCCACCTTCTCTAGGCACCGGATCAATTGCCGACCCCTCCC
CCCAACTTCTCGGGGACTGTGGGCGATGTGCGCTCTGCCCTAGTTGCTTGTGATTTCT
TTTCTTTTTTATTTTATTTCCATTATTTGAAATGTATTTGTTGTAGCAATGCTTTTGAC
ACGAAATAAATAAAAGAAAAAAAAAAAAAAAAAAAAAAAAAAAAAAAAAAAAAA
AAAAAAAAAAAAAAAAAAAAAAAAAAAAAAAAAAAAAAAAAA

Briefly, the template RNA for use with the Vingi-1_Acar gene modifying system comprises the following sequences from 5′ to 3′: 5′UTR, GFP, EF1alpha, 3′UTR and was co-delivered with the driver RNA. Vingi-1 Acar template RNA alone was used as negative controls. An RNA sample with high levels of double stranded RNA contaminants was used as a positive control.

The gene modifying systems and any control constructs were introduced as RNAs into activated T cells using Amaxa 4D-Nucleofector system.

Induction of interferon response was assessed using the following methodology. Briefly, cellular RNA was collected from activated T cells 24 hours after transfection with the RNAs of the gene modifying system or appropriate controls. Cellular IFNB1, CXCL10, and RPS18 RNA were quantified by quantitative RT-PCR. IFNB1 and CXCL10 are known markers of interferon response while RPS18 is a housekeeping gene commonly used as control for quantitative RT-PCR analysis.

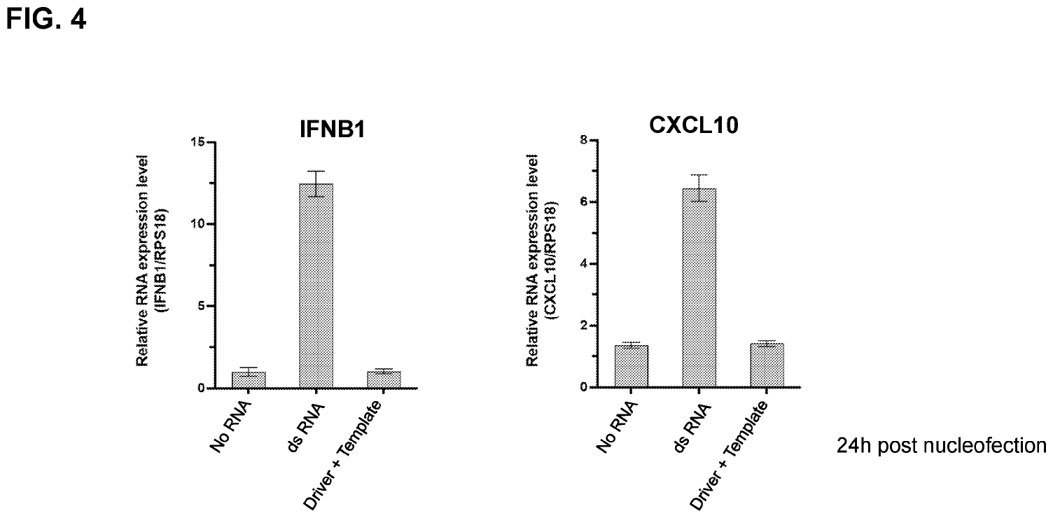

As shown in FIG. 4, introduction and editing using a gene modifying system did not activate interferon response in T cells. In contrast, introduction of high levels of dsRNA contaminants activated interferon response, as shown by the increased expression of IFNB1 and CXCL10 genes. The integration of a template sequence using gene modifying systems resulted in expression of up to 35% in T-cells (FIG. 3A) without activation of p53 or interferon responses.

### Example 4: Endonuclease Activity of Retrotransposon-Based Gene Modifying Systems

Residues potentially involved in endonuclease activity in a Vingi-1_Acar or CR1-1_PH driver were identified. Mutation D191A was introduced into the endonuclease domain of a wild type Vingi-1_Acar, and likewise D250A was introduced into the wild type CR1-1_PH endonuclease domain. The mutant polypeptides were tested for endonuclease activity. The wild type and mutant drivers were delivered to cells with a template including a transgene via plasmid nucleofection in order to compare the ability of the wild type and mutant protein to drive retrotransposition.

The wild type Vingi-1_Acar gene modifying system used in this Example comprises a nucleic acid encoding a Vingi-1_Acar driver having a nucleic acid sequence of:

(SEQ ID NO: 900)
atggacgagtaccagcgctccttgtcccgccccctgttgacaatcatgagtattaatatcgaaggcttgagcctcgcgaaagaggaactgctt
gccaagatgtccgaggacatcagctgcgacattctgtgcatccaagagacccatcgcgacatcaccatgcgtcgccccaagatcctcggta
tgcagcttgcggtggagcgccctcaccgccagtacggctccgcgattttcgtgcgttccggtgtggccatttctgctaccagcctcaccgag
gttaacaatattgagatcctgtcagtggagctcgattcctgtaccgtctccagtctgtacaaacctcccggtgcagacttctactttactcccc
cgacgtcttgccacaaccatgaggctcattttgtggttggtgatttcaattcccactcctgcgtgtggggctatgacgaggatgaccgcaacgg
cgaagctgttctgacctgggccgataattcccgcatgtccctgctccacgatagtaagctgcctccctccttcaacagtgggagatggaagcg
cgggtacaaccctgatcttatcttcgtcaaagaatccatctctcatcagtgtaccaagcgcgtcctcaacccgattcctaacactcagcaccgc
cccatttgttgcgtagcatatgctgccgtgcgtcccaagtctgtccccttcagaaggagatacaacttcaacaaggctaactggaccaagttta
ccgaaaccttggaagcggcaatctccgacattgagccatctatcgagaattatgatctcttcgttgaggcagtcaagcggtcctctcgcctga
gtatcccccgtggatgtaggacctcctacctgcccggcctgaacgaagagtcactcaatcagctgcaggaatacctgcgcctgttccaaga
gaatccttactccgatggcaccatcgctgccggacaaaaactgagcaccgctctggcgaatgctaagaaagaccgctggatcgagctgct
cgagaacctggacatgtccaagtctagccggaaagcttggcagctcctgcgccggctggacagcgaccccctcgtgaatcctggccatgc
caatgtaacgccggaccagatcgcccatcaactgatccagaacggcaagaccaattgcagtagaatcaagatgaagatcaacagagtacc
cgagttggaaacacaccagctgtctagtcctctcaacctcaaagagctccgcgaagcaatcaaaaggtgtaagacgggcaaagctcccgg
tctggatgacctgatgatggaacagatcaaacacttgggggccaaggctgagaactggttgctgaaattctacaaccagtgcctggctcaca
agcagatcccccgtgcgtggcgcaagactaagattatcgccatcttgaagccgggcaaggatgctagtaacgcccgcaactaccgtcctat
ttccctcctgtgtcacctttacaaggtgtacgagcgcatgctcctgaaccgcttgggccccgtcatcgagcctaaactgatcgcccaacaggc
tggtttccgcccaggtaagaactgcactggacagatcctgcaccttacggagcacatcgaggaaggttacgagaagggctgcatcaccgg
aaccgtcttcgtggacctgactgcggcctacgacacggttcagcacagaaaaatgctccacaaggtatatcatattacccgggatttcgactt
caccaaaacagtgcagacgcttctggagaatcgctccttttacgtcgagttccagggtcagaaaagtcggtggaggcgccagaagaacgg
cttgccccagggtagcgtgctcgccccaaccctgtttaatatcttcaccaacgaccagccacagcccccgctgacaaaatcctttatctatgc
cgacgatcttggcctgaccacgcaagccaaggactttgagacagtcgagaaacagctcacaaacgcactcaaggacctgagttcctattac
aaggagaaccatctcaaacctaaccccgctaagacacaggtctgcgcttttcacctgaggaatcgggaggccaaccgtaaactgaaggtg
acatgggagggacaggaactggagcactgtttccaccccaagtatttgggcgtgactctcgatcgcacccttacgtatcggaagcactgcat
gaacactaagcataaggtggcggccagaaataacatcctgcgtaaactcacaggaagcgcctggggcgccgacccacaggtcatccgta
ccagtgctctggccctgtctttctccactgctgaatatgcgtgtccggtgtggcacaagtccgcccacgcgaagcaggtggatattgccctga
acgagacatgccggattatcactggttgcctcaagcctacccccgtcgacaagctctataagttggccggcattgcaccccctgatgtccgtc
gcgaagtcgcagccaatggcgagcggaaaaaggtagaacactgcgaatcacatccgctgcatggatatcatccccctcccacccggctta
agagccgtaagggcttcatgcgcacaaccactccgctggatgtgcctcccgctgccgcgcgcgtgagtctgtgggccgctaagcccggta
attccaattggatggcaccgcaagagggcctcccgcccggagccaatcaagagtgggccacatggaagtccttgaacaggctgcgctcc
ggtgtgggacgctctaaggacaacctggcccgttggcactatctggaagagtcatccaccctgtgcgactgtggtgctgaacagactaccc
agcacatgtacgcttgcccccaatgccctgcctcctgcacggaagaggaactctttaaggcaaccgataacgcagtggcagtggcgcgctt
ttggagtaagacaatc

The wild type CR1-1PH gene modifying system used in this Example comprises a nucleic acid encoding a CR1-1_PH driver having a nucleic acid sequence of:

(SEQ ID NO: 901)
atgctgtataccatcttcctcgtttatgggatcctgtgttttttctttgtgtatttctgcatctacctgtacatcactgtttatctttgtttt
ctgttctttctgtttctgcttctgtgcggtgacgtggaaagcaatcctggaccaggcagagcccgtggatgtcggttgctgtattgcaacatc
cgcggtctgcatgctaatctggccgagctggactttgttagtcgcggggtggacgtggtctgctgttccgagactctggtggctgggcgtcgc
cacgatgccgagttggccctcgctggcttccagtccccctttcgtaggctctgtggctcaggccctggcttccgtggcatggcggtgtacgtc
aggtctgggttctgtgcctaccggcagagtgtgcacgaatgtgcctgccacgagattatcgtcgttcgcgtgtgtggacgtctgaacaatta
ctatctgttctctctctaccgctccccggccaccgatgactccctttacgattgccttctgactgccatggccagcatccagtccactgatcc
gaaggcagccttcgtcttcgttggcgacgtaaacgcacatcaccgggactggctgggaagcgcctctccaacagattgtcacggcgtcgccgc
tctggacttttgcaccctttccagctgcgtgcaactcgtccgtgggtccactcatatcgctggaaactgtctggatctggtgatgaccgacgt
gcctgacctgatgaccgtgaccgtcggatccccgattggaagtagcgaccactcccacctggtggtctcactggatctcaaccaggtcgtgcc
tgttgtggatacacgcagggttgtgttcgtgaagagccgcgccaactgggtcgccatcactcgcgctgtgaggactctgccttggcggcagat
tatccactccgaggaccctgtaagtgagctcaatgacctgaccgtgtctattctggaacgctttgttcccaagcgcaccatcctcgtgagaag
ccgcgataagccttggttcgatgaccagtgtagactggctttcgaggctaagcaggggcttaccgtgcttggcgtaggagtcgggaccgcacg
ttgtggcagacctatgtggacaggcgggccgaggccaagcgcgtgtatgaagaggctcagaggcgccttcgccagaggagccgtgaaagcctg
ctcagtattgaccaccctcatcgctggtggtcagagctcaagggctccgtgttcggcgccgaacccagcctgccccctctcgttggtcctggc
ggtggaatcattaccgatcccctcgccagggccgagctgctctccgcacattttgatggaaagcagtcacgcgatgtcatcgccttgccccac
ggttgccacccggagccccgcctcacatctctggccttcaggtccggcgctatcaaggtcctcctggaaggactcgacccctacggtggc
gttgaccccgtcggcatgtttcctctgttttacaagcagctggcggacgtcctggccccaaagttggccgttatctttcgtcggctgatccgc
ctcggcaactttccccgctgctggcgcactggtaatatcactcccatccccaagggccccgtgagtccttacgtggccaattaccgtcccatc
accttgacacctatcctgtctaaggttttcgaaaagctgatcgcaggcaagctgggtcgctttgctgaagtcactggcttgctccctgctggt
cagtttgcttatcgcatcggtctgggctgctgtgacgctctcctgtccgtgagccatcacctgcagtccgccctggactgccgctccgaagcc
cgcctggtgcagctcgatttctccgcagcgtttgaccgcgttaatcacaggggtcttctgtataaattggagtccttcggcgtcgggggccgg
gtgctgtcaattatccgtgatttcgtatccgagcgcacccagtctgtatccgtggacggtgttctgtccgccagcgtcggcgtggttagcgga
gtcccccagggtagcgtgctgggacctctgctctttgtattgtatacgtccgacatgttctctagcctggagaataccctcattaactatgca
gatgactccactttgatggcagttattccggccccacgcctgcgtgacgccgtagcgcagtccctgaatagggacctgtcacgcatcagtgcc
tggtgttcagcttggtccatgaagctgaacgcgtccaagaccaaaagcatgattatctcaaggtccaggactttggtgcctcaacacccccag
ctggagatcgacgatactttgctgcaggagtcctcatccctggaaatcctgggcgtcgtgttcgatgagaagctcaccttcgagcctcacatc
aggcggctggtgagccgcgcgtctaccaagatcgggctgctccgcaaggtgaactccgtgttcggcgattcccaggtcgcgaggcgttgcttt
tacgctttcctgctccctgtgctggagtactgctctccggtatgggcgagtgccgcggatacccatcttcgtcttctggaccgccttgtctcc
agcgccagccgtttgtgtgctgacaacgatctggtgaacctgagtcaccgtcggcgcgtggctgagctgtgtatgttctacaaggtgtacaat
aacgagaggcattggctgtactcatccctgccagccctgaaggtcttcgggcgcgaaacgcgtgccgcgtgcggtgctcactcttttacgttgg
aggcggttcgctgtcgcaccaaccagttctgccgctgctttgtgccttggtctgcgaaggtgtggaacctgctccctgcctccgctttcggga
gaatcggtctgcaggcatttaaaagcgctgtgaacggtttcctgttggactttctg

The wild-type Vingi-1_Acar driver encoded by the nucleic acid has an amino acid sequence of:

Vingi-1_Acar WT
(SEQ ID NO: 405)
MDEYQRSLSRPLLTIMSINIEGLSLAKEELLAKMSEDISCDILCIQETHRDITMRRPKILGM
QLAVERPHRQYGSAIFVRSGVAISATSLTEVNNIEILSVELDSCTVSSLYKPPGADFYFTPP
TSCHNHEAHFVVGDFNSHSCVWGYDEDDRNGEAVLTWADNSRMSLLHDSKLPPSFNS
GRWKRGYNPDLIFVKESISHQCTKRVLNPIPNTQHRPICCVAYAAVRPKSVPFRRRYNFN
KANWTKFTETLEAAISDIEPSIENYDLFVEAVKRSSRLSIPRGCRTSYLPGLNEESLNQLQ
EYLRLFQENPYSDGTIAAGQKLSTALANAKKDRWIELLENLDMSKSSRKAWQLLRRLD
SDPLVNPGHANVTPDQIAHQLIQNGKTNCSRIKMKINRVPELETHQLSSPLNLKELREAIK
RCKTGKAPGLDDLMMEQIKHLGAKAENWLLKFYNQCLAHKQIPRAWRKTKIIAILKPG
KDASNARNYRPISLLCHLYKVYERMLLNRLGPVIEPKLIAQQAGFRPGKNCTGQILHLTE
HIEEGYEKGCITGTVFVDLTAAYDTVQHRKMLHKVYHITRDFDFTKTVQTLLENRSFYV
EFQGQKSRWRRQKNGLPQGSVLAPTLFNIFTNDQPQPPLTKSFIYADDLGLTTQAKDFET
VEKQLTNALKDLSSYYKENHLKPNPAKTQVCAFHLRNREANRKLKVTWEGQELEHCF
HPKYLGVTLDRTLTYRKHCMNTKHKVAARNNILRKLTGSAWGADPQVIRTSALALSFS
TAEYACPVWHKSAHAKQVDIALNETCRIITGCLKPTPVDKLYKLAGIAPPDVRREVAAN
GERKKVEHCESHPLHGYHPPPTRLKSRKGFMRTTTPLDVPPAAARVSLWAAKPGNSNW
MAPQEGLPPGANQEWATWKSLNRLRSGVGRSKDNLARWHYLEESSTLCDCGAEQTTQ
HMYACPQCPASCTEEELFKATDNAVAVARFWSKTI

The wild-type CR1-1_PH driver encoded by the nucleic acid has an amino acid sequence of:

CR1-1_PH WT
(SEQ ID NO: 406)
MLYTIFLVYGILCFFFVYFCIYLYITVYLCFLFFLFLLLCGDVESNPGPGRARGCRLLYCNI
RGLHANLAELDFVSRGVDVVCCSETLVAGRRHDAELALAGFQSPFRRLCGSGPGFRGM
AVYVRSGFCAYRQSVHECACHEIIVVRVCGRLNNYYLFSLYRSPATDDSLYDCLLTAM
ASIQSTDPKAAFVFVGDVNAHHRDWLGSASPTDCHGVAALDFCTLSSCVQLVRGSTHIA
GNCLDLVMTDVPDLMTVTVGSPIGSSDHSHLVVSLDLNQVVPVVDTRRVVFVKSRAN
WVAITRAVRTLPWRQIIHSEDPVSELNDLTVSILERFVPKRTILVRSRDKPWFDDQCRLA
FEAKQAAYRAWRRSRDRTLWQTYVDRRAEAKRVYEEAQRRLRQRSRESLLSIDHPHR
WWSELKGSVFGAEPSLPPLVGPGGGIITDPLARAELLSAHFDGKQSRDVIALPHGCHPEP
RLTSLAFRSGAIKVLLEGLDPYGGVDPVGMFPLFYKQLADVLAPKLAVIFRRLIRLGNFP
RCWRTGNITPIPKGPVSPYVANYRPITLTPILSKVFEKLIAGKLGRFAEVTGLLPAGQFAY
RIGLGCCDALLSVSHHLQSALDCRSEARLVQLDFSAAFDRVNHRGLLYKLESFGVGGRV
LSIIRDFVSERTQSVSVDGVLSASVGVVSGVPQGSVLGPLLFVLYTSDMFSSLENTLINYA
DDSTLMAVIPAPRLRDAVAQSLNRDLSRISAWCSAWSMKLNASKTKSMIISRSRTL VPQ
HPQLEIDDTLLQESSSLEILGVVFDEKLTFEPHIRRLVSRASTKIGLLRKVNSVFGDSQVA
RRCFYAFLLPVLEYCSPVWASAADTHLRLLDRLVSSASRLCADNDLVNLSHRRRVAELC
MFYKVYNNERHWLYSSLPALKVFGRETRAACGAHSFTLEAVRCRTNQFCRCFVPWSA
KVWNLLPASAFGRIGLQAFKSAVNGFLLDFL

The mutated Vingi-1_Acar gene modifying system used in this Example comprises a nucleic acid encoding a mutated Vingi-1_Acar driver having a nucleic acid sequence of:

(SEQ ID NO: 902)
atggacgagtaccagcgctccttgtcccgccccctgttgacaatcatgagtattaatatcgaaggcttgagcctcgcgaaagaggaactgctt
gccaagatgtccgaggacatcagctgcgacattctgtgcatccaagagacccatcgcgacatcaccatgcgtcgccccaagatcctcggta
tgcagcttgcggtggagcgccctcaccgccagtacggctccgcgattttcgtgcgttccggtgtggccatttctgctaccagcctcaccgag
gttaacaatattgagatcctgtcagtggagctcgattcctgtaccgtctccagtctgtacaaacctcccggtgcagacttctactttactcccc
cgacgtcttgccacaaccatgaggctcattttgtggttggtgatttcaattcccactcctgcgtgtggggctatgacgaggatgaccgcaacgg
cgaagctgttctgacctgggccgataattcccgcatgtccctgctccacgatagtaagctgcctccctccttcaacagtgggagatggaagcg
cgggtacaaccctgcccttatcttcgtcaaagaatccatctctcatcagtgtaccaagcgcgtcctcaacccgattcctaacactcagcaccg
ccccatttgttgcgtagcatatgctgccgtgcgtcccaagtctgtccccttcagaaggagatacaacttcaacaaggctaactggaccaagttt
accgaaaccttggaagcggcaatctccgacattgagccatctatcgagaattatgatctcttcgttgaggcagtcaagcggtcctctcgcctg
agtatcccccgtggatgtaggacctcctacctgcccggcctgaacgaagagtcactcaatcagctgcaggaatacctgcgcctgttccaag
agaatccttactccgatggcaccatcgctgccggacaaaaactgagcaccgctctggcgaatgctaagaaagaccgctggatcgagctgc
tcgagaacctggacatgtccaagtctagccggaaagcttggcagctcctgcgccggctggacagcgaccccctcgtgaatcctggccatg
ccaatgtaacgccggaccagatcgcccatcaactgatccagaacggcaagaccaattgcagtagaatcaagatgaagatcaacagagtac
ccgagttggaaacacaccagctgtctagtcctctcaacctcaaagagctccgcgaagcaatcaaaaggtgtaagacgggcaaagctcccg
gtctggatgacctgatgatggaacagatcaaacacttgggggccaaggctgagaactggttgctgaaattctacaaccagtgcctggctcac
aagcagatcccccgtgcgtggcgcaagactaagattatcgccatcttgaagccgggcaaggatgctagtaacgcccgcaactaccgtcct
atttccctcctgtgtcacctttacaaggtgtacgagcgcatgctcctgaaccgcttgggccccgtcatgagcctaaactgatcgcccaacag
gctggtttccgcccaggtaagaactgcactggacagatcctgcaccttacggagcacatcgaggaaggttacgagaagggctgcatcacc
ggaaccgtcttcgtggacctgactgcggcctacgacacggttcagcacagaaaaatgctccacaaggtatatcatattacccgggatttcga
cttcaccaaaacagtgcagacgcttctggagaatcgctccttttacgtcgagttccagggtcagaaaagtcggtggaggcgccagaagaac
ggcttgccccagggtagcgtgctcgccccaaccctgtttaatatcttcaccaacgaccagccacagcccccgctgacaaaatcctttatctat
gccgacgatcttggcctgaccacgcaagccaaggactttgagacagtcgagaaacagctcacaaacgcactcaaggacctgagttcctatt
acaaggagaaccatctcaaacctaaccccgctaagacacaggtctgcgcttttcacctgaggaatcgggaggccaaccgtaaactgaagg
tgacatgggagggacaggaactggagcactgtttccaccccaagtatttgggcgtgactctcgatcgcacccttacgtatcggaagcactgc
atgaacactaagcataaggtggcggccagaaataacatcctgcgtaaactcacaggaagcgcctggggcgccgacccacaggtcatccg
taccagtgctctggccctgtctttctccactgctgaatatgcgtgtccggtgtggcacaagtccgcccacgcgaagcaggtggatattgccct
gaacgagacatgccggattatcactggttgcctcaagcctacccccgtcgacaagctctataagttggccggcattgcaccccctgatgtcc
gtcgcgaagtcgcagccaatggcgagcggaaaaaggtagaacactgcgaatcacatccgctgcatggatatcatccccctcccacccgg
cttaagagccgtaagggcttcatgcgcacaaccactccgctggatgtgcctcccgctgccgcgcgcgtgagtctgtgggccgctaagccc
ggtaattccaattggatggcaccgcaagagggcctcccgcccggagccaatcaagagtgggccacatggaagtccttgaacaggctgcg
ctccggtgtgggacgctctaaggacaacctggcccgttggcactatctggaagagtcatccaccctgtgcgactgtggtgctgaacagact
acccagcacatgtacgcttgcccccaatgccctgcctcctgcacggaagaggaactctttaaggcaaccgataacgcagtggcagtggcg
cgcttttggagtaagacaatc

The mutated CR1-1_PH gene modifying system used in this Example comprises a nucleic acid encoding a mutated CR1-1_PH driver having a nucleic acid sequence of:

(SEQ ID NO: 903)
atgctgtataccatcttcctcgtttatgggatcctgtgttttttctttgtgtatttctgcatctacctgtacatcactgtttatctttgtttt
ctgttctttctgtttctgcttctgtgcggtgacgtggaaagcaatcctggaccaggcagagcccgtggatgtcggttgctgtattgcaacatc
cgcggtctgcatgctaatctggccgagctggactttgttagtcgcggggtggacgtggtctgctgttccgagactctggtggctgggcgtcgc
cacgatgccgagttggccctcgctggcttccagtccccctttcgtaggctctgtggctcaggccctggcttccgtggcatggcggtgtacgtc
aggtctgggttctgtgcctaccggcagagtgtgcacgaatgtgcctgccacgagattatcgtcgttcgcgtgtgtggacgtctgaacaattac
tatctgttctctctctaccgctccccggccaccgatgactccctttacgattgccttctgactgccatggccagcatccagtccactgatccg
aaggcagccttcgtcttcgttggcgacgtaaacgcacatcaccgggactggctgggaagcgcctctccaacagattgtcacggcgtcgccgc
tctggacttttgcaccctttccagctgcgtgcaactcgtccgtgggtccactcatatcgctggaaactgtctggatctggtgatgaccgacgt
gcctgccctgatgaccgtgaccgtcggatccccgattggaagtagcgaccactcccacctggtggtctcactggatctcaaccaggtcgtgcc
tgttgtggatacacgcagggttgtgttcgtgaagagccgcgccaactgggtcgccatcactcgcgctgtgaggactctgccttggggcagatt
atccactccgaggaccctgtaagtgagctcaatgacctgaccgtgtctattctggaacgctttgttcccaagcgcaccatcctcgtgagaagc
cgcgataagccttggttcgatgaccagtgtagactggctttcgaggctaagcaggcggcttaccgtgcttggcgtaggagtcgggaccgcacg
ttgtggcagacctatgtggacaggcgggccgaggccaagcgcgtgtatgaagaggctcagaggcgccttcgccagaggagccgtgaaagcctg
ctcagtattgaccaccctcatcgctggtggtcagagctcaagggctccgtgttcggcgccgaacccagcctgccccctctcgttggtcctggc
ggtggaatcattaccgatcccctcgccagggccgagctgctctccgcacattttgatggaaagcagtcacgcgatgtcatcgccttgccccac
ggttgccacccggagccccgcctcacatctctggccttcaggtccggcgctatcaaggtcctcctggaaggactcgacccctacggtggc
gttgaccccgtcggcatgtttcctctgttttacaagcagctggcggacgtcctggccccaaagttggccgttatctttcgtcggctgatccgc
ctcggcaactttccccgctgctggcgcactggtaatatcactcccatccccaagggccccgtgagtccttacgtggccaattaccgtcccatc
accttgacacctatcctgtctaaggttttcgaaaagctgatcgcaggcaagctgggtcgctttgctgaagtcactggcttgctccctgctggt
cagtttgcttatcgcatcggtctgggctgctgtgacgctctcctgtccgtgagccatcacctgcagtccgccctggactgccgctccgaagcc
cgcctggtgcagctcgatttctccgcagcgtttgaccgcgttaatcacaggggtcttctgtataaattggagtccttcggcgtcgggggccgg
gtgctgtcaattatccgtgatttcgtatccgagcgcacccagtctgtatccgtggacggtgttctgtccgccagcgtcggcgtggttagcgga
gtcccccagggtagcgtgctgggacctctgctctttgtattgtatacgtccgacatgttctctagcctggagaataccctcattaactatgca
gatgactccactttgatggcagttattccggccccacgcctgcgtgacgccgtagcgcagtccctgaatagggacctgtcacgcatcagtgcc
tggtgttcagcttggtccatgaagctgaacgcgtccaagaccaaaagcatgattatctcaaggtccaggactttggtgcctcaacacccccag
ctggagatcgacgatactttgctgcaggagtcctcatccctggaaatcctgggcgtcgtgttcgatgagaagctcaccttcgagcctcacatc
aggcggctggtgagccgcgcgtctaccaagatcgggctgctccgcaaggtgaactccgtgttcggcgattcccaggtcgcgaggcgttgcttt
tacgctttcctgctccctgtgctggagtactgctctccggtatgggcgagtgccgcggatacccatcttcgtcttctggaccgccttgtctcc
agcgccagccgtttgtgtgctgacaacgatctggtgaacctgagtcaccgtcggcgcgtggctgagctgtgtatgttctacaaggtgtacaat
aacgagaggcattggctgtactcatccctgccagccctgaaggtcttcgggcgcgaaacgcgtgccgcgtgcggtgctcactcttttacgttg
gaggcggttcgctgtcgcaccaaccagttctgccgctgctttgtgccttggtctgcgaaggtgtggaacctgctccctgcctccgctttcggg
agaatcggtctgcaggcatttaaaagcgctgtgaacggtttcctgttggactttctg

The mutated Vingi-1_Acar driver encoded by the nucleic acid sequence has an amino acid sequence of:

Vingi-1_Acar EN (D191A)
(SEQ ID NO: 420)
MDEYQRSLSRPLLTIMSINIEGLSLAKEELLAKMSEDISCDILCIQETHRDITMRRPKILGM
QLAVERPHRQYGSAIFVRSGVAISATSLTEVNNIEILSVELDSCTVSSLYKPPGADFYFTPP
TSCHNHEAHFVVGDFNSHSCVWGYDEDDRNGEAVLTWADNSRMSLLHDSKLPPSFNS
GRWKRGYNPALIFVKESISHQCTKRVLNPIPNTQHRPICCVAYAAVRPKSVPFRRRYNFN
KANWTKFTETLEAAISDIEPSIENYDLFVEAVKRSSRLSIPRGCRTSYLPGLNEESLNQLQ
EYLRLFQENPYSDGTIAAGQKLSTALANAKKDRWIELLENLDMSKSSRKAWQLLRRLD
SDPLVNPGHANVTPDQIAHQLIQNGKTNCSRIKMKINRVPELETHQLSSPLNLKELREAIK
RCKTGKAPGLDDLMMEQIKHLGAKAENWLLKFYNQCLAHKQIPRAWRKTKIIAILKPG
KDASNARNYRPISLLCHLYKVYERMLLNRLGPVIEPKLIAQQAGFRPGKNCTGQILHLTE
HIEEGYEKGCITGTVFVDLTAAYDTVQHRKMLHKVYHITRDFDFTKTVQTLLENRSFYV
EFQGQKSRWRRQKNGLPQGSVLAPTLFNIFTNDQPQPPLTKSFIYADDLGLTTQAKDFET
VEKQLTNALKDLSSYYKENHLKPNPAKTQVCAFHLRNREANRKLKVTWEGQELEHCF
HPKYLGVTLDRTLTYRKHCMNTKHKVAARNNILRKLTGSAWGADPQVIRTSALALSFS
TAEYACPVWHKSAHAKQVDIALNETCRIITGCLKPTPVDKLYKLAGIAPPDVRREVAAN
GERKKVEHCESHPLHGYHPPPTRLKSRKGFMRTTTPLDVPPAAARVSLWAAKPGNSNW
MAPQEGLPPGANQEWATWKSLNRLRSGVGRSKDNLARWHYLEESSTLCDCGAEQTTQ
HMYACPQCPASCTEEELFKATDNAVAVARFWSKTI

The mutated CR1-1_PH driver encoded by the nucleic acid sequence has an amino acid sequence of:

CR1-1_PH EN (D250A):
(SEQ ID NO: 421)
MLYTIFLVYGILCFFFVYFCIYLYITVYLCFLFFLFLLLCGDVESNPGPGRARGCRLLYCNI
RGLHANLAELDFVSRGVDVVCCSETLVAGRRHDAELALAGFQSPFRRLCGSGPGFRGM
AVYVRSGFCAYRQSVHECACHEIIVVRVCGRLNNYYLFSLYRSPATDDSLYDCLLTAM
ASIQSTDPKAAFVFVGDVNAHHRDWLGSASPTDCHGVAALDFCTLSSCVQLVRGSTHIA
GNCLDLVMTDVPALMTVTVGSPIGSSDHSHLVVSLDLNQVVPVVDTRRVVFVKSRAN
WVAITRAVRTLPWRQIIHSEDPVSELNDLTVSILERFVPKRTILVRSRDKPWFDDQCRLA
FEAKQAAYRAWRRSRDRTLWQTYVDRRAEAKRVYEEAQRRLRQRSRESLLSIDHPHR
WWSELKGSVFGAEPSLPPLVGPGGGIITDPLARAELLSAHFDGKQSRDVIALPHGCHPEP
RLTSLAFRSGAIKVLLEGLDPYGGVDPVGMFPLFYKQLADVLAPKLAVIFRRLIRLGNFP
RCWRTGNITPIPKGPVSPYVANYRPITLTPILSKVFEKLIAGKLGRFAEVTGLLPAGQFAY
RIGLGCCDALLSVSHHLQSALDCRSEARLVQLDFSAAFDRVNHRGLLYKLESFGVGGRV
LSIIRDFVSERTQSVSVDGVLSASVGVVSGVPQGSVLGPLLFVLYTSDMFSSLENTLINYA
DDSTLMAVIPAPRLRDAVAQSLNRDLSRISAWCSAWSMKLNASKTKSMIISRSRTLVPQ
HPQLEIDDTLLQESSSLEILGVVFDEKLTFEPHIRRLVSRASTKIGLLRKVNSVFGDSQVA
RRCFYAFLLPVLEYCSPVWASAADTHLRLLDRLVSSASRLCADNDLVNLSHRRRVAELC
MFYKVYNNERHWLYSSLPALKVFGRETRAACGAHSFTLEAVRCRTNQFCRCFVPWSA
KVWNLLPASAFGRIGLQAFKSAVNGFLLDFL

Both the wild type and the mutated Vingi-1_Acar gene modifying system further comprises a template plasmid containing a transgene with a sequence of:

(SEQ ID NO: 1001)
gggggacacggaaagagcctccccgaagattgagtgaattcagtcgggcgtcccctgggcaacgtttcttgtaagcggccgatctttccac
cccaaaagcattggatgagtttacggatccgaattctcgacggatcgatccgaacaaacgacccaacacccgtgcgttttattctgtcttttta
ttgccgatcccccggccgctttacttgtacagctcgtccatgccgagagtgatcccggcggcggtcacgaactccagcaggaccatgtgatc
gcgcttctcgttggggtctttgctcagggcggactgggtgctcaggtaagtatcaaggttacaagacaggtttaaggagaccaatagaaact
gggcttgtcgagacagagaagactcttgcgtttctgataggcacctattggtcttactgacatccactttgcctttctctccacaggtagtggt
tgtcgggcagcagcacggggccgtcgccgatgggggtgttctgctggtagtggtcggcgagctgcacgctgccgtcctcgatgttgtggcgg
atcttgaagttcaccttgatgccgttcttctgcttgtcggccatgatatagacgttgtggctgttgtagttgtactccagcttgtgccccagga
tgttgccgtcctccttgaagtcgatgcccttcagctcgatgcggttcaccagggtgtcgccctcgaacttcacctcggcgcgggtcttgtagtt
gccgtcgtccttgaagaagatggtgcgctcctggacgtagccttcgggcatggcggacttgaagaagtcgtgctgcttcatgtggtcggggtag
cggctgaagcactgcacgccgtaggtcagggtggtcacgagggtgggccagggcacgggcagcttgccggtggtgcagatgaacttcag
ggtcagcttgccgtaggtggcatcgccctcgccctcgccggacacgctgaacttgtggccgtttacgtcgccgtccagctcgaccaggatg
ggcaccaccccggtgaacagctcctcgcccttgctcaccatggtggctttaccaacagtaccggaatgccaagcttgggtcctgtgttctgg
cggcaaacccgttgcgaaaaagaacgttcacggcgactactgcacttatatacggttctcccccaccctcgggaaaaaggcggagccagt
acacgacatcactttcccagtttaccccgcgccaccttctctaggcaccggatcaattgccgacccctccccccaacttctcggggactgtgg
gcgatgtgcgctctgccctagttgcttgtgatttcttttcttttttattttatttccattatttgaaatgtatttgttgtagcaatgctttt
gacacgaaataaataaa

In SEQ ID NO: 1001, the underlined sequences indicate, from 5′ to 3′, the 5′ UTR, GFPai, EF1 alpha (each with single underline), and 3′ UTR (with double underline).

Both the wild-type and the mutated CR1-1_PH gene modifying system further comprises a template plasmid containing a transgene with a sequence of:

(SEQ ID NO: 1002)
gcgtggcctgcgcgttatcaggccgcccccattgtccggccaccggacgcctctttgtttgtatgagctggctttgggtttggggctctgggt
agcccctgagttggaggaaatgccactctaaaggcggatgagacctcttttggtccaacggcatccctgagaggagatgaacaggctggt
gccatagctgggtactcagtcttccgcagtctgctcctctctctcttccatcaggtagtaccacctgatgctcttttcctttcctttctcttct
tcttcttttcggccaaagcctgtactggtgatggcagggtctggggcgcgtagatagctggccgctttcgtagctatcgttactgtcgatttgt
ttttgctttattgtttacggatccgaattctcgacggatcgatccgaacaaacgacccaacacccgtgcgttttattctgtctttttattgccg
atcccccggccgctttacttgtacagctcgtccatgccgagagtgatcccggcggcggtcacgaactccagcaggaccatgtgatcgcgcttct
cgttggggtctttgctcagggcggactggctgctcaggtaagtatcaaggttacaagacaggtttaaggagaccaatagaaactgggcttgtcg
agacagagaagactcttgcgtttctgataggcacctattggtcttactgacatccactttgcctttctctccacaggtagtggttgtcgggcag
cagcacggggccgtcgccgatgggggtgttctgctggtagtggtcggcgagctgcacgctgccgtcctcgatgttgtggcggatcttgaagttc
accttgatgccgttcttctgcttgtcggccatgatatagacgttgtggctgttgtagttgtactccagcttgtgccccaggatgttgccgtcct
ccttgaagtcgatgcccttcagctcgatgcggttcaccagggtgtcgccctcgaacttcacctcggcgcgggtcttgtagttgccgtcgtcctt
gaagaagatggtgcgctcctggacgtagccttcgggcatggcggacttgaagaagtcgtgctgcttcatgtggtcggggtagcggctgaagcac
tgcacgccgtaggtcagggtggtcacgagggtgggccagggcacgggcagcttgccggtggtgcagatgaacttcagggtcagcttgccgta
ggtggcatcgccctcgccctcgccggacacgctgaacttgtggccgtttacgtcgccgtccagctcgaccaggatgggcaccaccccggt
gaacagctcctcgcccttgctcaccatggtggctttaccaacagtaccggaatgccaagcttgggtcctgtgttctggcggcaaacccgttgc
gaaaaagaacgttcacggcgactactgcacttatatacggttctcccccaccctcgggaaaaaggcggagccagtacacgacatcactttc
ccagtttaccccgcgccaccttctctaggcaccggatcaattgccgacccctccccccaacttctcggggactgtgggcgatgtgcgctctg
ccctagcagcgttttgggcttcccggtgttccaagatgcttcatttgggttgtattacctgtccgtgggagctcaagcgtgaagtttta
ataataataataa

In SEQ ID NO: 1002, the underlined sequences indicate, from 5′ to 3′, the 5′ UTR, GFPai, EF1 alpha (each with single underline), and 3′ UTR (with double underline).

Briefly, the template RNA encoded by the plasmid comprises the following sequences from 5′ to 3′: 5′UTR, GFPai reporter driven by EF1-alpha promoter, and 3′ UTR.

U2OS cells were transfected with both a driver and a transgene plasmid using a Lonza nucleofection system. 250 ng of DNA/400,000 cells in a ratio of 2:1 transgene to driver plasmid was used. Cells were plated in a 24 well plate and then incubated at 37C, 5% CO2 for 3 days. Two sets of three bio-replicates for each combination of driver:transgene plasmids and controls were prepared.

The first set of replicates was subjected to ddPCR analysis to detect integration events. Integration was assessed using the following methodology on day 3 following nucleofection: genomic DNA extraction was performed followed by ddPCR analysis to identify copies of DNA spanning the reverse transcribed and integrated GFPai splice site junction. The second set of replicates was processed for flow cytometry analysis to measure GFP activity.

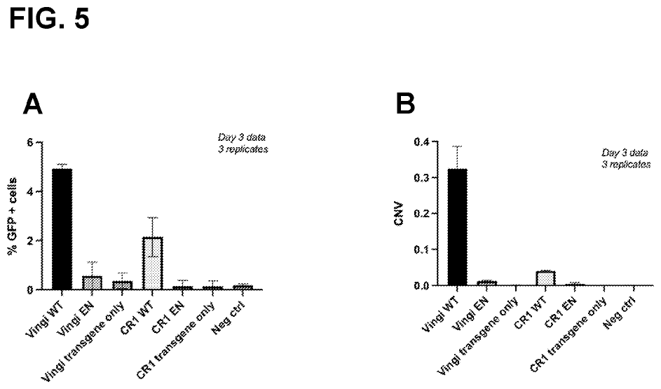

As shown in FIG. 5, integration was observed in cells that were treated with gene modifying systems comprising a wild type Vingi-1_Acar or CR1-1_PH driver, while those treated with a mutated Vingi-1_Acar or CR1-1_PH driver demonstrated background levels of integration as measured via flow cytometry as an indicator of transgene expression or quantifying integrated DNA by ddPCR. These results demonstrate that endonuclease activity contributes to integration of a heterologous object sequence using gene modifying systems. Furthermore, these data show that a genome editing system including the D191A or D250A mutant driver shows near background levels of integration activity.

### Example 5: Gene Editing of Primary Human T Cells with a BCMA CAR by a Retrotransposon-Based Gene Modifying System Results in CART Cells that can Kill Target Tumor Cells

This example demonstrates that gene modifying systems may be used to introduce a chimeric antigen receptor (CAR) molecule into the genome into primary human T cells. The resultant CART cell can kill target tumor cells at least as well as a control CART cell derived from a Lentivirus.

The gene modifying system used in this Example comprises a nucleic acid encoding Vingi-1_Acar driver having a nucleic acid sequence of:

(SEQ ID NO: 904)
atggacgagtaccagcgctccttgtcccgccccctgttgacaatcatgagtattaatatcgaaggcttgagcctcgcgaaagaggaactgctt
gccaagatgtccgaggacatcagctgcgacattctgtgcatccaagagacccatcgcgacatcaccatgcgtcgccccaagatcctcggta
tgcagcttgcggtggagcgccctcaccgccagtacggctccgcgattttcgtgcgttccggtgtggccatttctgctaccagcctcaccgag
gttaacaatattgagatcctgtcagtggagctcgattcctgtaccgtctccagtctgtacaaacctcccggtgcagacttctactttactcccc
cgacgtcttgccacaaccatgaggctcattttgtggttggtgatttcaattcccactcctgcgtgtggggctatgacgaggatgaccgcaacgg
cgaagctgttctgacctgggccgataattcccgcatgtccctgctccacgatagtaagctgcctccctccttcaacagtgggagatggaagcg
cgggtacaaccctgatcttatcttcgtcaaagaatccatctctcatcagtgtaccaagcgcgtcctcaacccgattcctaacactcagcaccgc
cccatttgttgcgtagcatatgctgccgtgcgtcccaagtctgtccccttcagaaggagatacaacttcaacaaggctaactggaccaagttta
ccgaaaccttggaagcggcaatctccgacattgagccatctatcgagaattatgatctcttcgttgaggcagtcaagcggtcctctcgcctga
gtatcccccgtggatgtaggacctcctacctgcccggcctgaacgaagagtcactcaatcagctgcaggaatacctgcgcctgttccaaga
gaatccttactccgatggcaccatcgctgccggacaaaaactgagcaccgctctggcgaatgctaagaaagaccgctggatcgagctgct
cgagaacctggacatgtccaagtctagccggaaagcttggcagctcctgcgccggctggacagcgaccccctcgtgaatcctggccatgc
caatgtaacgccggaccagatcgcccatcaactgatccagaacggcaagaccaattgcagtagaatcaagatgaagatcaacagagtacc
cgagttggaaacacaccagctgtctagtcctctcaacctcaaagagctccgcgaagcaatcaaaaggtgtaagacgggcaaagctcccgg
tctggatgacctgatgatggaacagatcaaacacttgggggccaaggctgagaactggttgctgaaattctacaaccagtgcctggctcaca
agcagatcccccgtgcgtggcgcaagactaagattatcgccatcttgaagccgggcaaggatgctagtaacgcccgcaactaccgtcctat
ttccctcctgtgtcacctttacaaggtgtacgagcgcatgctcctgaaccgcttgggccccgtcatcgagcctaaactgatcgcccaacaggc
tggtttccgcccaggtaagaactgcactggacagatcctgcaccttacggagcacatcgaggaaggttacgagaagggctgcatcaccgg
aaccgtcttcgtggacctgactgcggcctacgacacggttcagcacagaaaaatgctccacaaggtatatcatattacccgggatttcgactt
caccaaaacagtgcagacgcttctggagaatcgctccttttacgtcgagttccagggtcagaaaagtcggtggaggcgccagaagaacgg
cttgccccagggtagcgtgctcgccccaaccctgtttaatatcttcaccaacgaccagccacagcccccgctgacaaaatcctttatctatgc
cgacgatcttggcctgaccacgcaagccaaggactttgagacagtcgagaaacagctcacaaacgcactcaaggacctgagttcctattac
aaggagaaccatctcaaacctaaccccgctaagacacaggtctgcgcttttcacctgaggaatcgggaggccaaccgtaaactgaaggtg
acatgggagggacaggaactggagcactgtttccaccccaagtatttgggcgtgactctcgatcgcacccttacgtatcggaagcactgcat
gaacactaagcataaggtggcggccagaaataacatcctgcgtaaactcacaggaagcgcctggggcgccgacccacaggtcatccgta
ccagtgctctggccctgtctttctccactgctgaatatgcgtgtccggtgtggcacaagtccgcccacgcgaagcaggtggatattgccctga
acgagacatgccggattatcactggttgcctcaagcctacccccgtcgacaagctctataagttggccggcattgcaccccctgatgtccgtc
gcgaagtcgcagccaatggcgagcggaaaaaggtagaacactgcgaatcacatccgctgcatggatatcatccccctcccacccggctta
agagccgtaagggcttcatgcgcacaaccactccgctggatgtgcctcccgctgccgcgcgcgtgagtctgtgggccgctaagcccggta
attccaattggatggcaccgcaagagggcctcccgcccggagccaatcaagagtgggccacatggaagtccttgaacaggctgcgctcc
ggtgtgggacgctctaaggacaacctggcccgttggcactatctggaagagtcatccaccctgtgcgactgtggtgctgaacagactaccc
agcacatgtacgcttgcccccaatgccctgcctcctgcacggaagaggaactctttaaggcaaccgataacgcagtggcagtggcgcgctt
ttggagtaagacaatcggcggcggctcccccaagaagaagcggaaggtgagcggcagcgagactcccgggacctcagagtccgccac
acccgaaagtgtgagcggctggcggctgttcaagaagattagc

The gene modifying system used in this Example comprises a nucleic acid encoding 3GS Linker having a nucleic acid sequence of:

(SEQ ID NO: 15437)
ggcggcggctcc

The gene modifying system used in this Example comprises a nucleic acid encoding an SV40 NLS having a nucleic acid sequence of:

(SEQ ID NO: 15438)
cccaagaagaagcggaaggtg

The gene modifying system used in this Example comprises a nucleic acid encoding an XTEN linker having a nucleic acid sequence of:

(SEQ ID NO: 15439)
agcggcagcgagactcccgggacctcagagtccgccacacccgaaagt

The gene modifying system used in this Example comprises a nucleic acid encoding an HiBit tag having a nucleic acid sequence of:

(SEQ ID NO: 15440)
gtgagcggctggcggctgttcaagaagattagc

The Vingi-1_Acar driver encoded by the nucleic acid has an amino acid sequence of:

(SEQ ID NO: 405)
MDEYQRSLSRPLLTIMSINIEGLSLAKEELLAKMSEDISCDILCIQETHRDITMRRPKILGM
QLAVERPHRQYGSAIFVRSGVAISATSLTEVNNIEILSVELDSCTVSSLYKPPGADFYFTPP
TSCHNHEAHFVVGDFNSHSCVWGYDEDDRNGEAVLTWADNSRMSLLHDSKLPPSFNS
GRWKRGYNPDLIFVKESISHQCTKRVLNPIPNTQHRPICCVAYAAVRPKSVPFRRRYNFN
KANWTKFTETLEAAISDIEPSIENYDLFVEAVKRSSRLSIPRGCRTSYLPGLNEESLNQLQ
EYLRLFQENPYSDGTIAAGQKLSTALANAKKDRWIELLENLDMSKSSRKAWQLLRRLD
SDPLVNPGHANVTPDQIAHQLIQNGKTNCSRIKMKINRVPELETHQLSSPLNLKELREAIK
RCKTGKAPGLDDLMMEQIKHLGAKAENWLLKFYNQCLAHKQIPRAWRKTKIIAILKPG
KDASNARNYRPISLLCHLYKVYERMLLNRLGPVIEPKLIAQQAGFRPGKNCTGQILHLTE
HIEEGYEKGCITGTVFVDLTAAYDTVQHRKMLHKVYHITRDFDFTKTVQTLLENRSFYV
EFQGQKSRWRRQKNGLPQGSVLAPTLFNIFTNDQPQPPLTKSFIYADDLGLTTQAKDFET
VEKQLTNALKDLSSYYKENHLKPNPAKTQVCAFHLRNREANRKLKVTWEGQELEHCF
HPKYLGVTLDRTLTYRKHCMNTKHKVAARNNILRKLTGSAWGADPQVIRTSALALSFS
TAEYACPVWHKSAHAKQVDIALNETCRIITGCLKPTPVDKLYKLAGIAPPDVRREVAAN
GERKKVEHCESHPLHGYHPPPTRLKSRKGFMRTTTPLDVPPAAARVSLWAAKPGNSNW
MAPQEGLPPGANQEWATWKSLNRLRSGVGRSKDNLARWHYLEESSTLCDCGAEQTTQ
HMYACPQCPASCTEEELFKATDNAVAVARFWSKTI

The gene modifying system used in this Example comprises a nucleic acid encoding 3GS (SEQ ID NO: 15441) Linker has an amino acid sequence of:

(SEQ ID NO: 15442)
GGGS

The gene modifying system used in this Example comprises a nucleic acid encoding an SV40 NLS has an amino acid sequence of:

(SEQ ID NO: 345)
PKKKRKV

The gene modifying system used in this Example comprises a nucleic acid encoding an XTEN linker has an amino acid sequence of:

(SEQ ID NO: 220)
SGSETPGTSESATPES

The gene modifying system used in this Example comprises a nucleic acid encoding an HiBit tag has an amino acid sequence of:

(SEQ ID NO: 15443)
VSGWRLFKKIS

The Vingi-1_Acar gene modifying system further comprises an RNA that serves as a template for encoding a BCMA-CAR-T2A-GFP comprising a nucleic acid sequence of:

(SEQ ID NO: 1003)
atggctctgccggtgaccgccctgcttctgcctcttgccctgctcttgcatgccgctcgcccgcaggtgcagcttgttcagtccggcgcgga
ggtaaaaaagcctggcgccagcgtgaaagtgagctgtaaggcttccggctacagcttcccagattactatatcaattgggtgcggcaggct
cccgggcagggactcgagtggatgggttggatctattttgcgagcggaaactctgagtacaaccagaagttcaccggcagggttaccatga
cccgcgacacgtcaagttctacggcatacatggagctgtctagtctcaggtccgaagataccgccgtgtatttctgcgcctccctgtatgatta
tgactggtacttcgacgtctggggccagggcaccatggtgacagtgtcttccggtggcggtggctccggcggaggtggctctggagggg
gtggcagcgatatcgtgatgacccagactccactgtctctgtctgtgacccctggtgagccggcgagcatctcctgcaagagttcccagagt
ctggtccactctaacggcaacacctacctgcactggtatctgcagaagccgggccaaagtccacagctcctgatctacaaggtttccaaccg
tttcagcggggtcccggatcgcttttccggctcaggtagcggtgccgatttcaccctgaagattagtcgggtcgaggctgaggacgtcggc
gtctactattgtgcagagaccagtcacgtcccctggactttcggtcaggggaccaaactggagatcaagaccacaacacctgctccaaggc
cccccacacccgctccaactatagccagccaaccattgagcctcagacctgaagcttgcaggcccgcagcaggaggcgccgtccatacg
cgaggcctggacttcgcgtgtgatatttatatttgggcccctttggccggaacatgtggggtgttgcttctctcccttgtgatcactctgtatt
gtaggagtaagaggagcaggctcctgcacagtgactacatgaacatgactccccgccgccccgggcccacccgcaagcattaccagccctat
gccccaccacgcgacttgcagcctatcgctccagggtgaaattttctagaagcgccgatgctcccgcatatcagcagggtcagaatcagc
tctacaatgaattgaatctcggcaggcgagaagagtacgatgttctggacaagagacggggcagggatcccgagatggggggaaagccc
cggagaaaaaatcctcaggaggggttgtacaatgagctgcagaaggacaagatggctgaagcctatagcgagatcggaatgaaaggcg
aaagacgcagaggcaaggggcatgacggtctgtaccagggtctctctacagccaccaaggacacttatgatgcgttgcatatgcaagcctt
gccaccccgcggaagtggtgagggcagaggaagtcttctaacatgcggtgacgtggaggagaatcccggccctatgcttctcctggtgac
aagccttctgctctgtgagttaccacacccagcattcctcctgatcccacgcaaagtgtgtaacggaataggtattggtgaatttaaagactca
ctctccataaatgctacgaatattaaacacttcaaaaactgcacctccatcagtggcgatctccacatcctgccggtggcatttaggggtgact
ccttcacacatactcctcctctggatccacaggaactggatattctgaaaaccgtaaaggaaatcacagggtttttgctgattcaggcttggcct
gaaaacaggacggacctccatgcctttgagaacctagaaatcatacgcggcaggaccaagcaacatggtcagttttctcttgcagtcgtcag
cctgaacataacatccttgggattacgctccctcaaggagataagtgatggagatgtgataatttcaggaaacaaaaatttgtgctatgcaaat
acaataaactggaaaaaactgtttgggacctccggtcagaaaaccaaaattataagcaacagaggtgaaaacagctgcaaggccacaggc
caggtctgccatgccttgtgctcccccgagggctgctggggcccggagcccagggactgcgtctcttgccggaatgtcagccgaggcag
ggaatgcgtggacaagtgcaaccttctggagggtgagccaagggagtttgtggagaactctgagtgcatacagtgccacccagagtgcct
gcctcaggccatgaacatcacctgcacaggacggggaccagacaactgtatccagtgtgcccactacattgacggcccccactgcgtcaa
gacctgcccggcaggagtcatgggagaaaacaacaccctggtctggaagtacgcagacgccggccatgtgtgccacctgtgccatccaa
actgcacctacggatgcactgggccaggtcttgaaggctgtccaacgaatgggcctaagatcccgtccatcgccactgggatggtggggg
ccctcctcttgctgctggtggtggccctggggatcggcctcttcatg

The gene modifying system used in this Example comprises a nucleic acid encoding a CD8 hinge domain having a nucleic acid sequence of:

(SEQ ID NO: 15444)
accacaacacctgctccaaggccccccacacccgctccaactatagcca
gccaaccattgagcctcagacctgaagcttgcaggcccgcagcaggagg
cgccgtccatacgcgaggcctggacttcgcgtgtgat

The gene modifying system used in this Example comprises a nucleic acid encoding a CD8 transmembrane domain having a nucleic acid sequence of:

(SEQ ID NO: 15445)
atttatatttgggcccctttggccggaacatgtggggtgttgcttctct
cccttgtgatcactctgtattgt

The gene modifying system used in this Example comprises a nucleic acid encoding a CD28 co-stimulatory domain having a nucleic acid sequence of:

(SEQ ID NO: 15446)
aggagtaagaggagcaggctcctgcacagtgactacatgaacatgactc
cccgccgccccgggcccacccgcaagcattaccagccctatgccccacc
acgcgacttcgcagcctatcgctcc

The gene modifying system used in this Example comprises a nucleic acid encoding a CD3z signaling domain having a nucleic acid sequence of:

(SEQ ID NO: 15447)
agggtgaaattttctagaagcgccgatgctcccgcatatcagcagggtc
agaatcagctctacaatgaattgaatctcggcaggcgagaagagtacga
tgttctggacaagagacggggcagggatcccgagatggggggaaagccc
cggagaaaaaatcctcaggaggggttgtacaatgagctgcagaaggaca
agatggctgaagcctatagcgagatcggaatgaaaggcgaaagacgcag
aggcaaggggcatgacggtctgtaccagggtctctctacagccaccaag
gacacttatgatgcgttgcatatgcaagccttgccaccccgc

The gene modifying system used in this Example comprises a nucleic acid encoding a GSG Linker having a nucleic acid sequence of:

ggaagtggt

The gene modifying system used in this Example comprises a nucleic acid encoding a T2A peptide having a nucleic acid sequence of:

(SEQ ID NO: 15448)
gagggcagaggaagtcttctaacatgcggtgacgtggaggagaatcccg
gccct

The gene modifying system used in this Example comprises a nucleic acid encoding a GFP protein having a nucleic acid sequence of:

(SEQ ID NO: 1004)
Atggtgagcaagggcgaggagctgttcaccggggtggtgcccatcctggtcgagctggacggcgacgtaaacggccacaagttcagcg
tgtccggcgagggcgagggcgatgccacctacggcaagctgaccctgaagttcatctgcaccaccggcaagctgcccgtgccctggccc
accctcgtgaccaccctgacctacggcgtgcagtgcttcagccgctaccccgaccacatgaagcagcacgacttcttcaagtccgccatgc
ccgaaggctacgtccaggagcgcaccatcttcttcaaggacgacggcaactacaagacccgcgccgaggtgaagttcgagggcgacac
cctggtgaaccgcatcgagctgaagggcatcgacttcaaggaggacggcaacatcctggggcacaagctggagtacaactacaacagcc
acaacgtctatatcatggccgacaagcagaagaacggcatcaaggtgaacttcaagatccgccacaacatcgaggacggcagcgtgcag
ctcgccgaccactaccagcagaacacccccatcggcgacggccccgtgctgctgcccgacaaccactacctgagcacccagtccgccct
gagcaaagaccccaacgagaagcgcgatcacatggtcctgctggagttcgtgaccgccgccgggatcactctcggcatggacgagctgt
acaag

The Vingi-1_Acar gene modifying system further comprises an RNA that serves as a template for encoding a BCMA-CAR-T2A-GFP comprising a protein sequence of:

(SEQ ID NO: 1100)
MALPVTALLLPLALLLHAARPQVQLVQSGAEVKKPGASVKVSCKASGYS
FPDYYINWVRQAPGQGLEWMGWIYFASGNSEYNQKFTGRVTMTRDTSSS
TAYMELSSLRSEDTAVYFCASLYDYDWYFDVWGQGTMVTVSSGGGGSGG
GGSGGGGSDIVMTQTPLSLSVTPGEPASISCKSSQSLVHSNGNTYLHWY
LQKPGQSPQLLIYKVSNRFSGVPDRFSGSGSGADFTLKISRVEAEDVGV
YYCAETSHVPWTFGQGTKLEIKTTTPAPRPPTPAPTIASQPLSLRPEAC
RPAAGGAVHTRGLDFACDIYIWAPLAGTCGVLLLSLVITLYCRSKRSRL
LHSDYMNMTPRRPGPTRKHYQPYAPPRDFAAYRSRVKFSRSADAPAYQQ
GQNQLYNELNLGRREEYDVLDKRRGRDPEMGGKPRRKNPQEGLYNELQK
DKMAEAYSEIGMKGERRRGKGHDGLYQGLSTATKDTYDALHMQALPPRG
SGEGRGSLLTCGDVEENPGPMLLLVTSLLLCELPHPAFLLIPRKVCNGI
GIGEFKDSLSINATNIKHFKNCTSISGDLHILPVAFRGDSFTHTPPLDP
QELDILKTVKEITGFLLIQAWPENRTDLHAFENLEIIRGRTKQHGQFSL
AVVSLNITSLGLRSLKEISDGDVIISGNKNLCYANTINWKKLFGTSGQK
TKIISNRGENSCKATGQVCHALCSPEGCWGPEPRDCVSCRNVSRGRECV
DKCNLLEGEPREFVENSECIQCHPECLPQAMNITCTGRGPDNCIQCAHY
IDGPHCVKTCPAGVMGENNTLVWKYADAGHVCHLCHPNCTYGCTGPGLE
GCPTNGPKIPSIATGMVGALLLLLVVALGIGLFM

The gene modifying system used in this Example comprises a nucleic acid encoding a CD8 hinge domain having a protein sequence of:

(SEQ ID NO: 1101)
TTTPAPRPPTPAPTIASQPLSLRPEACRPAAGGAVHTRGLDFACD

The gene modifying system used in this Example comprises a nucleic acid encoding a CD8 transmembrane domain having a protein sequence of:

(SEQ ID NO: 1102)
IYIWAPLAGTCGVLLLSLVITLYC

The gene modifying system used in this Example comprises a nucleic acid encoding a CD28 co-stimulatory domain having a protein sequence of:

(SEQ ID NO: 1103)
RSKRSRLLHSDYMNMTPRRPGPTRKHYQPYAPPRDFAAYRS

The gene modifying system used in this Example comprises a nucleic acid encoding a CD3z signaling domain having a protein sequence of:

(SEQ ID NO: 1104)
RVKFSRSADAPAYQQGQNQLYNELNLGRREEYDVLDKRRGRDPEMGGK
PRRKNPQEGLYNELQKDKMAEAYSEIGMKGERRRGKGHDGLYQGLSTA
TKDTYDALHMQALPPR

The gene modifying system used in this Example comprises a nucleic acid encoding a GSG Linker having a protein sequence of:

GSG

The gene modifying system used in this Example comprises a nucleic acid encoding a T2A peptide having a protein sequence of:

(SEQ ID NO: 1106)
EGRGSLLTCGDVEENPGP

The gene modifying system used in this Example comprises a nucleic acid encoding a GFP protein having a protein sequence of:

(SEQ ID NO: 1107)
MVSKGEELFTGVVPILVELDGDVNGHKFSVSGEGEGDATYGKLTLKFIC
TTGKLPVPWPTLVTTLTYGVQCFSRYPDHMKQHDFFKSAMPEGYVQERT
IFFKDDGNYKTRAEVKFEGDTLVNRIELKGIDFKEDGNILGHKLEYNYN
SHNVYIMADKQKNGIKVNFKIRHNIEDGSVQLADHYQQNTPIGDGPVLL
PDNHYLSTQSALSKDPNEKRDHMVLLEFVTAAGITLGMDELYK

Briefly, the template RNA for use with the Vingi-1_Acar gene modifying system comprises the following sequences from 5′ to 3′: 5′ UTR, bGHpA, WPRE, BCMA-CAR-T2A-GFP, Kozak sequence, MND promoter, 3′ UTR.

The Vingi-1_Acar gene modifying system and BCMA-CAR-T2A-GFP template RNA was introduced into primary human T cells. Briefly, a vial of human T cells stored at liquid nitrogen from one donor was thawed and placed into culture media containing TexMACS supplemented with 5% human serum albumin. Twenty-four hours after thaw, T cells were activated by supplementing the culture media with TransACT (according to manufacturer's protocol; Miltenyi) IL2 (20 IU/mL; Miltenyi), IL7 (10 ng/mL; Miltenyi), and IL15 (5 ng/mL; Miltenyi) for two days. Cells were counted daily to ensure viability above 80%.

On the second day of T cell activation, T cells were either electroporated with Vingi-1_Acar and CAR RNA or transduced with a Lentivirus encoding the same CAR. As a control, just the CAR template was electroporated into T cells. For the electroporation, a total of 50 million T cells were electroporated with 500 μg/mL of RNA (mass ratio of 2 template:1 driver) in a volume of 400 uLs of OptiMEM. Electroporations were quenched in 400 uLs of OptiMEM for ten minutes at 37C followed by addition expansion media (TexMACS, 10 ng/mL of IL7, 5 ng/mL of IL15, 5% human serum albumin). Electroporated T cells were expanded and analyzed by flow cytometry CAR and GFP expression for 5, 7, and 9 days.

As a comparison, the same BCMA-CAR-T2A-GFP molecule was introduced into donor matched T cells using a lentiviral gene delivery system as follows. After two days of T cell activation, a Lentiviral vector encoding BCMA-CAR-T2A-GFP was diluted ten-fold and added to T cells without serum for 24 hours and then replaced with expansion culture media (TexMACS, 10 ng/mL of IL7, 5 ng/mL of IL15, 5% human serum albumin).

On day 9 of the experiment 100 million GFP+/CAR+ cells were sorted and enriched from Vingi-1_Acar-edited cells. Expression of GFP+ cells in this population was 0.5% and sorting enriched the GFP fraction to 50% of the total T cell population. Lentiviral transduction yielded 10% GFP+/CAR+ cells and to equilibrate cell numbers for the cytotoxicity assay, Vingi-1 Acar-edited CART cells were back-diluted with donor matched non-electroporated control cells.

BCMA-CART cells were then co-cultured for 24 hours with BCMA-positive tumor cells (H929) at various T cell to tumor ratios. Both tumor cells and CART cells were then counted by flow cytometry and killing percentages assessed by this equation: % Killing=1−(number of target cells/number of target cells in control)*100.

As shown in FIGS. 6A-6C, successful expression of the CAR molecule into the T cells was achieved using the gene modifying system. More specifically, at the lowest ratio of CAR:tumor cells, the gene modifying system resulted in 30% killing compared to 10% cell killing by lentivirus-produced cells. At an intermediate ratio of CAR:tumor cells, the gene modifying system resulted in 45% killing compared to 20% cell killing by lentivirus-produced cells. At the highest ratio of CAR:tumor cells, the gene modifying system resulted in 60% killing compared to 35% cell killing by lentivirus-produced cells. This data indicates that BCMA-CART cells derived from the Vingi-1_Acar gene modifying system are at least as effective as Lentiviral derived CART cells in the functional capacity to kill tumor cells.

### Example 6: Gene Editing of Primary Human T Cells with a BCMA CAR by a Retrotransposon-Based Gene Modifying System Results in CART Cells that can Kill Target Tumor Cells

This example demonstrates that exemplary gene modifying systems may be used to introduce a chimeric antigen receptor (CAR) molecule into the genome into primary human T cells. The resultant CART cell can kill target tumor cells.

The gene modifying system used in this Example comprised a nucleic acid encoding an exemplary RTE1_MD gene modifying polypeptide comprising an amino acid sequence of:

(SEQ ID NO: 402)
MDSTAHPNQGRGLEKVSQTLPALQTPGQHTAAGGSSPLSGRNQRKNTKK
LLLGAWNIRTLLDRENTPRPERRTALIGKELARYNIDIAALSETRLPEE
GSLSEPTTGYTFFWKGRASNEDRIHGVGLAIKTSLLKQLPDLPVGISER
LMKIRLPLSKDRYATIISAYAPTLTSTEETIEQFYSDLSAVLHSVPTND
KLILLGDFNARVGQDHERWKGVLGKHGVGKMNNNGLLLLSKCSEFELTI
TNTVFRMANKYKTTWMHPRSKQWHLIDYIIVRRRDIQDVKITRAMRGAE
CWTDHRLVRATLQMRIAPRHPKRAQTVRAFYNVSRLRDPSYLQTFQSCL
DDKLSAKGPLTGSSTEKWNQFRDAVKETSKAVLGPKQRNHQDWFDENNT
AIEDLLSKKNKAFMEWQNNPNSAPKKDRFKSLQATAQREIRKMQDRWWE
KKAEEIQRFADMKNYKQFFSALKTVYGPLKPTTTPLLSSDGDTLIKDKK
GISNRWKEHFSQLLNRPSSVDQSALDQIPQNRTIEQLDVPPSIEEVQKA
IKQMSAGKAPGKDGIPTEVYKALNGKALQAFHIVLTSIWEEEDMPPELR
DASIVALYKNKGSRAACDNYRGISLLSTAGKILARVILNRLLSSVSEQN
LPESQCGFRPDRSTIDMVFTVRQMQEKCLEQNLSLYIVFIDLTKAFDTV
NRDALWVILSKLGCPAKFVKLIQLFHVDMTGEVLSGGETSDRFNISNGV
KQGCVLAPVLFNLFFTQVLRHAVMDLDLGVYIKYRLDGSLFDLRRLTAK
TKTTERLILEALFADDCALMAHQENHLQTIVDRFSTATKLFGLTISLSK
TEVLFQPAPGRPTNQPCITIDGTQLSNVNTFKYLGSTIANDGSLDHEIN
ARIQKASQALGRLRCKVLQHRGVSTATKLKVYNAVVLSSLLYGCETWTL
YRKHMKQLEQFHQRSLRSIMRIRWQDRITNQEVLDRANSTSIEVMVLKT
QLRWSGHVIRMDPQRIPRQVFYGELSAGLRKQGRPKKRFKDQLKSNLKW
AGITPKQLELAASDRSSWRTHINHAATTFEDERRRRLAAARERRHQATT
APPVTTGVPCPMCHKLCASAFGLQSHMRVHRR

The gene modifying polypeptide comprised a 3GS (SEQ ID NO: 15441) Linker having an amino acid sequence of:

(SEQ ID NO: 15442)
GGGS

The gene modifying polypeptide comprised an SV40 NLS having an amino acid sequence of:

(SEQ ID NO: 345)
PKKKRKV

The gene modifying polypeptide comprised an XTEN linker having an amino acid sequence of:

(SEQ ID NO: 220)
SGSETPGTSESATPES

The gene modifying polypeptide used in this Example comprised an HiBit tag having an amino acid sequence of:

(SEQ ID NO: 15443)
VSGWRLFKKIS

The gene modifying system further comprised a template RNA that encoded a BCMA-CAR fusion polypeptide, the template RNA comprising a nucleic acid sequence of:

(SEQ ID NO: 15449)
ccggcgcggagguaaaaaagccuggcgccagcgugaaagugagcuguaaggcuuccggcuacagcuucccagauuacuauau
caauugggugcggcaggcucccgggcagggacucgaguggauggguuggaucuauuuuggagcggaaacucugaguacaa
ccagaaguucaccggcaggguuaccaugacccgcgacacgucaaguucuacggcauacauggagcugucuagucucaggucc
gaagauaccgccguguauuucugcgccucccuguaugauuaugacugguacuucgacgucuggggccagggcaccaugguga
cagugucuuccggugggguggcuccggggagguggcucuggaggggguggcagcgauaucgugaugacccagacuccac
ugucucugucugugaccccuggugagccggcgagcaucuccugcaagaguucccagagucugguccacucuaacggcaacac
cuaccugcacugguaucugcagaagccgggccaaaguccacagcuccugaucuacaagguuuccaaccguuucagcggggucc
cggaucgcuuuuccggcucagguagcggugccgauuucacccugaagauuagucgggucgaggcugaggacgucggcgucu
acuauugugcagagaccagucacguccccuggacuuucggucaggggaccaaacuggagaucaagaccacaacaccugcucca
aggccccccacacccgcuccaacuauagccagccaaccauugagccucagaccugaagcuugcaggcccgcagcaggaggcgcc
ucccuugugaucacucuguauuguaggaguaagaggagcaggcuccugcacagugacuacaugaacaugacuccccgccgcc
ccgggcccacccgcaagcauuaccagcccuaugccccaccacgcgacuucgcagccuaucgcuccagggugaaauuuucuaga
agcgccgaugcucccgcauaucagcagggucagaaucagcucuacaaugaauugaaucucggcaggcgagaagaguacgaug
uucuggacaagagacggggcagggaucccgagauggggggaaagccccggagaaaaaauccucaggagggguuguacaauga
gcugcagaaggacaagauggcugaagccuauagcgagaucggaaugaaaggcgaaagacgcagaggcaaggggcaugacggu
cuguaccagggucucucuacagccaccaaggacacuuaugaugcguugcauaugcaagccuugccaccccgc

The BCMA-CAR fusion polypeptide comprised an amino acid sequence of:

(SEQ ID NO: 15450)
MALPVTALLLPLALLLHAARPQVQLVQSGAEVKKPGASVKVSCKASGY
SFPDYYINWVRQAPGQGLEWMGWIYFASGNSEYNQKFTGRVTMTRDTS
SSTAYMELSSLRSEDTAVYFCASLYDYDWYFDVWGQGTMVTVSSGGGG
SGGGGSGGGGSDIVMTQTPLSLSVTPGEPASISCKSSQSLVHSNGNTY
LHWYLQKPGQSPQLLIYKVSNRFSGVPDRFSGSGSGADFTLKISRVEA
EDVGVYYCAETSHVPWTFGQGTKLEIKTTTPAPRPPTPAPTIASQPLS
LRPEACRPAAGGAVHTRGLDFACDIYIWAPLAGTCGVLLLSLVITLYC
RSKRSRLLHSDYMNMTPRRPGPTRKHYQPYAPPRDFAAYRSRVKFSRS
ADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDA
LHMQALPPR

The BCMA-CAR fusion polypeptide comprised a CD8 hinge domain having an amino acid sequence of:

(SEQ ID NO: 1101)
TTTPAPRPPTPAPTIASQPLSLRPEACRPAAGGAVHTRGLDFACD

The BCMA-CAR fusion polypeptide comprised a CD8 transmembrane domain having an amino acid sequence of:

(SEQ ID NO: 1102)
IYIWAPLAGTCGVLLLSLVITLYC

The BCMA-CAR fusion polypeptide comprised a CD28 co-stimulatory domain having an amino acid sequence of:

(SEQ ID NO: 1103)
RSKRSRLLHSDYMNMTPRRPGPTRKHYQPYAPPRDFAAYRS

The BCMA-CAR fusion polypeptide comprised a CD3z signaling domain having an amino acid sequence of:

(SEQ ID NO: 1104)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

The BCMA-CAR fusion polypeptide comprised a GSG Linker having an amino acid sequence of:

GSG

The BCMA-CAR fusion polypeptide comprised a T2A peptide having an amino acid sequence of:

(SEQ ID NO: 1106)
EGRGSLLTCGDVEENPGP

Briefly, the template RNA for use with the RTE1_MD gene modifying system had the following sequences from 5′ to 3′:

5′ UTRᵣₑₜᵣₒ, bGH polyA, WPRE, BCMA-CAR, Kozak sequence, MND promoter, 3′ UTRᵣₑₜᵣₒ.

The RTE1_MD gene modifying system comprising a BCMA-CAR template RNA was introduced into primary human CD3+ T cells. Briefly, human CD3+ T cells stored in liquid nitrogen from one donor were thawed in TexMACS culture media (Miltenyi) supplemented with 5% Human AB serum, IL2 (20 IU/mL; Miltenyi), IL7 (10 ng/mL; Miltenyi), and IL15 (5 ng/mL; Miltenyi). Cells were activated immediately post thaw by supplementing the culture media with TransACT (according to manufacturer's protocol) for two days.

Activated T cells were electroporated with mRNA encoding RTE1_MD gene modifying polypeptide and BCMA CAR template RNA. As a control, T cells that underwent no genetic modification were cultured along with cells that underwent the electroporated conditions. For the electroporation, a total of 500-700 million T cells were electroporated with 500 μg/mL of RNA (mass ratio of 200 template:1 driver) in a volume of 400-450 uLs of OptiMEM to maintain a cell concentration of 4×10⁸ cells/mL Electroporations were quenched in expansion media (TexMACS, 100 IU/mL IL2, 10 ng/mL of IL7, 5 ng/mL of IL15, 5% Human AB serum). Electroporated T cells were expanded until day 10 post thaw and analyzed by flow cytometry for CAR expression on days 7 and 10 post-thaw. 100 IU/mL IL2 was also added to the cell culture on these days to promote cell expansion.

On day 10, CART cells were harvested and function was assessed via a BCMA target cell killing assay. Briefly, BCMA-CART cells were co-cultured for 24 hours with BCMA-positive tumor cell lines (H929) at serially decreasing T cell (effector) to tumor cell ratios. Cells from co-culture were stained with antibodies to uniquely identify surface markers for both target cells and effector cells. Stained co-cultures were acquired on a flow cytometer with dead cell exclusion via a vital dye. Targets were positively identified during analysis and absolute counts of target cells for each condition were generated by volumetric readout by the flow cytometer. All conditions were normalized to a target only well to control for tumor growth via the following equation: % Killing=1−(number of target cells in test condition/number of target cells in normalization control)*100. After 24 hours culture supernatants were assessed for IFNγ and Granzyme B concentration via ELISA.

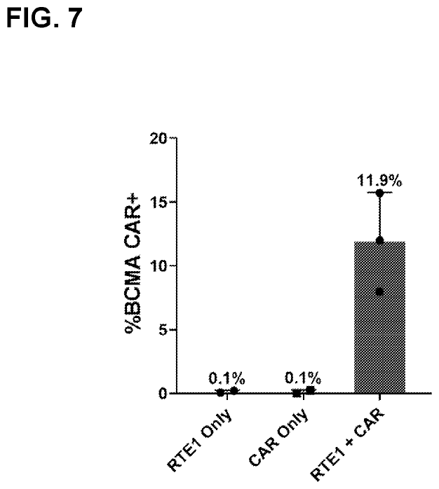

As shown in FIG. 7, BCMA-CAR, introduced by the RTE1-MD gene modifying system, expressed in human primary T cells obtained from three different donors. Activated T cells were co-electroporated with both RTE1_MD gene modifying polypeptide and the BCMA CAR template and assessed for CAR expression 3 days post electroporation. The average CAR expression observed was 12% and the highest expression of BCMA CAR from one donor observed was 15%. In comparison, cells treated with either mRNA encoding RTE1_MD gene modifying polypeptide alone or BCMA CAR template alone did not show any BCMA CAR expression. These results demonstrate that RTE1-MD gene modifying systems can be used to insert and express a therapeutic transgene (a BCMA CAR) into human primary T cells.

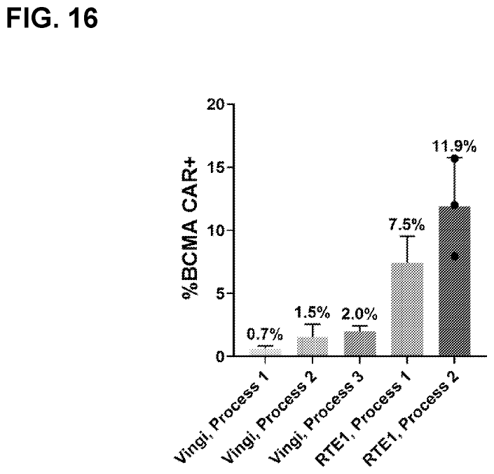

As shown in FIG. 8A, primary human T cells from two donors that express BCMA-CAR (as delivered by RTE1_MD gene modifying system) killed BCMA positive tumor cells in a dose dependent manner. Over 50% killing was seen at a 0.625 ratio of effector T cells to tumor cells for one donor, and approximately 73-85% killing was observed for both donors with a 2.5 or 5 ratio of effector T cells to tumor cells. As shown in FIG. 8B, increased IFNγ production and Granzyme B release were seen with increasing concentrations of CART cells to tumor bearing targets. Both are biomarkers for cytotoxicity of CART cells against tumor cells. These data, taken together, indicate that the BCMA CAR T cells produced using RTE1_MD gene modifying systems represent a functional and potent CART product that controlled tumor growth in vitro. BCMA-CAR was also introduced into human primary T cells using the Vingi-1_Acar gene modifying system described herein or the RTE1-MD gene modifying system described herein using different processes (FIG. 16). For Vingi-1_Acar process two, the gene modifying system used an mRNA comprising N1-Methylpseudouridine as an alternative chemical modification. This change resulted in an increase average expression of 0.7% to 1.5% BCMA CAR. Vingi-1_Acar process three incubated the primary T cells at 30° C. 24 hours post electroporation of the Vingi-1_Acar gene modifying system. This resulted in a modest increase in BCMA CAR editing and expression. Use of RTE1-MD gene modifying system improved BCMA CAR expression to 7.5% (process 1). RTE1-MD gene modifying system process two electroporated mRNA encoding RTE1-MD gene modifying polypeptide and BCMA CAR template at a mass ratio of 1 to 200 while process one was 1:25. The process two ratio dramatically increased expression of BCMA CAR relative to process one (and further described above).

### Example 7: Gene Editing of Primary Human T Cells with a BCMA CAR by a Retrotransposon-Based Gene Modifying System Results in CART Cells that can Kill Target Tumor Cells

This example demonstrates that exemplary gene modifying systems may be used to introduce a chimeric antigen receptor (CAR) molecule into the genome of primary human T cells. The resultant CART cell can kill target tumor cells.

The gene modifying system used in this Example comprises a nucleic acid encoding an exemplary Vingi-1_Acar gene modifying polypeptide comprising an amino acid sequence of:

(SEQ ID NO: 405)
MDEYQRSLSRPLLTIMSINIEGLSLAKEELLAKMSEDISCDILCIQETH
RDITMRRPKILGMQLAVERPHRQYGSAIFVRSGVAISATSLTEVNNIEI
LSVELDSCTVSSLYKPPGADFYFTPPTSCHNHEAHFVVGDFNSHSCVWG
YDEDDRNGEAVLTWADNSRMSLLHDSKLPPSFNSGRWKRGYNPDLIFVK
ESISHQCTKRVLNPIPNTQHRPICCVAYAAVRPKSVPFRRRYNFNKANW
TKFTETLEAAISDIEPSIENYDLFVEAVKRSSRLSIPRGCRTSYLPGLN
EESLNQLQEYLRLFQENPYSDGTIAAGQKLSTALANAKKDRWIELLENL
DMSKSSRKAWQLLRRLDSDPLVNPGHANVTPDQIAHQLIQNGKTNCSRI
KMKINRVPELETHQLSSPLNLKELREAIKRCKTGKAPGLDDLMMEQIKH
LGAKAENWLLKFYNQCLAHKQIPRAWRKTKIIAILKPGKDASNARNYRP
ISLLCHLYKVYERMLLNRLGPVIEPKLIAQQAGFRPGKNCTGQILHILT
EHIEEGYEKGCITGTVFVDLTAAYDTVQHRKMLHKVYHITRDFDFTKTV
QTLLENRSFYVEFQGQKSRWRRQKNGLPQGSVLAPTLFNIFTNDQPQPP
LTKSFIYADDLGLTTQAKDFETVEKQLTNALKDLSSYYKENHLKPNPAK
TQVCAFHLRNREANRKLKVTWEGQELEHCFHPKYLGVTLDRTLTYRKHC
MNTKHKVAARNNILRKLTGSAWGADPQVIRTSALALSFSTAEYACPVWH
KSAHAKQVDIALNETCRIITGCLKPTPVDKLYKLAGIAPPDVRREVAAN
GERKKVEHCESHPLHGYHPPPTRLKSRKGFMRTTTPLDVPPAAARVSLW
AAKPGNSNWMAPQEGLPPGANQEWATWKSLNRLRSGVGRSKDNLARWHY
LEESSTLCDCGAEQTTQHMYACPQCPASCTEEELFKATDNAVAVARFWS
KTI

The gene modifying polypeptide comprised a 3GS (SEQ ID NO: 15441) Linker having an amino acid sequence of:

(SEQ ID NO: 15442)
GGGS

The gene modifying polypeptide comprised an SV40 NLS having an amino acid sequence of:

(SEQ ID NO: 345)
PKKKRKV

The gene modifying polypeptide comprised an XTEN linker having an amino acid sequence of:

(SEQ ID NO: 220)
SGSETPGTSESATPES

The gene modifying polypeptide used in this Example comprised an HiBit tag having an amino acid sequence of:

(SEQ ID NO: 15443)
VSGWRLFKKIS

The gene modifying system further comprises a template RNA that encoded a BCMA-CAR-T2A-RQR8 fusion polypeptide, the template RNA comprising a nucleic acid sequence of:

(SEQ ID NO: 15451)
ccggcgcggagguaaaaaagccuggcgccagcgugaaagugagcuguaaggcuuccggcuacagcuucccagauuacuauau
caauugggugcggcaggcucccgggcagggacucgaguggauggguuggaucuauuuugcgagcggaaacucugaguacaa
ccagaaguucaccggcaggguuaccaugacccgegacacgucaaguucuacggcauacauggagcugucuagucucaggucc
gaagauaccgccguguauuucugcgccucccuguaugauuaugacugguacuucgagucuggggccagggcaccaugguga
cagugucuuccgguggcgguggcuccggggagguggcucuggaggggguggcagegauaucgugaugacccagacuccac
ugucucugucugugaccccuggugagccggegagcaucuccugcaagaguucccagagucugguccacucuaacggcaacac
cuaccugcacugguaucugcagaagccgggccaaaguccacagcuccugaucuacaagguuuccaaccguuucagcggggucc
cggaucgcuuuuccggcucagguagcggugccgauuucacccugaagauuagugggucgaggcugaggacguggcgucu
acuauugugcagagaccagucacguccccuggacuuucggucaggggaccaaacuggagaucaagaccacaacaccugcucca
aggccccccacacccgcuccaacuauagccagccaaccauugagccucagaccugaagcuugcaggcccgcagcaggaggcgcc
ucccuugugaucacucuguauuguaggaguaagaggagcaggcuccugcacagugacuacaugaacaugacuccccgccgcc
ccgggcccaccegcaagcauuaccagcccuaugccccaccacgcgacuucgcagccuaucgcuccagggugaaauuuucuaga
agcgccgaugcucccgcauaucagcagggucagaaucagcucuacaaugaauugaaucucggcaggcgagaagaguacgaug
uucuggacaagagacggggcagggaucccgagauggggggaaagccccggagaaaaaauccucaggagggguuguacaauga
gcugcagaaggacaagauggcugaagccuauagcgagaucggaaugaaaggcgaaagacgcagaggcaaggggcaugacggu
cuguaccagggucucucuacagccaccaaggacacuuaugaugcguugcauaugcaagccuugccaccccgcggaaguggug
agggcagaggaagucuucuaacaugcggugacguggaggagaaucccggcccuauggggacgucacuccuguguuggaugg
ccuugugccuccugggagcugaccacgcugacgcaugccccuacagcaacccuagcuugugcagcggugggguggguccga
acugccuacccaggggacuuuuucuaacguauccaccaacgugucccccgccaagcccacuacaaccgccugcccauacuccaa
ucucugcgcccggaagcgugucggcccgcggcuggggggccgugcacaccagaggccuggacuucgccugcgacaucuaca
ucugggccccauuggcagguaccugcggcguacugcuccugagucugguuauuacucuguacugcaaccaccggaacaggag
acgcgucuguaagugcccuagaccggugguguaa

The BCMA-CAR-T2A-RQR8 fusion polypeptide comprised an amino acid sequence of:

(SEQ ID NO: 15452)
MALPVTALLLPLALLLHAARPQVQLVQSGAEVKKPGASVKVSCKASGYS
FPDYYINWVRQAPGQGLEWMGWIYFASGNSEYNQKFTGRVTMTRDTSSS
TAYMELSSLRSEDTAVYFCASLYDYDWYFDVWGQGTMVTVSSGGGGSGG
GGSGGGGSDIVMTQTPLSLSVTPGEPASISCKSSQSLVHSNGNTYLHWY
LQKPGQSPQLLIYKVSNRFSGVPDRFSGSGSGADFTLKISRVEAEDVGV
YYCAETSHVPWTFGQGTKLEIKTTTPAPRPPTPAPTIASQPLSLRPEAC
RPAAGGAVHTRGLDFACDIYIWAPLAGTCGVLLLSLVITLYCRSKRSRL
LHSDYMNMTPRRPGPTRKHYQPYAPPRDFAAYRSRVKFSRSADAPAYQQ
GQNQLYNELNLGRREEYDVLDKRRGRDPEMGGKPRRKNPQEGLYNELQK
DKMAEAYSEIGMKGERRRGKGHDGLYQGLSTATKDTYDALHMQALPPRG
SGEGRGSLLTCGDVEENPGPMGTSLLCWMALCLLGADHADACPYSNPSL
CSGGGGSELPTQGTFSNVSTNVSPAKPTTTACPYSNPSLCSGGGGSPAP
RPPTPAPTIASQPLSLRPEACRPAAGGAVHTRGLDFACDIYIWAPLAGT
CGVLLLSLVITLYCNHRNRRRVCKCPRPVV*

The BCMA-CAR-T2A-RQR8 fusion polypeptide comprised a CD8 hinge domain having an amino acid sequence of:

(SEQ ID NO: 1101)
TTTPAPRPPTPAPTIASQPLSLRPEACRPAAGGAVHTRGLDFACD

The BCMA-CAR-T2A-RQR8 fusion polypeptide comprised a CD8 transmembrane domain having an amino acid sequence of:

(SEQ ID NO: 1102)
IYIWAPLAGTCGVLLLSLVITLYC

The BCMA-CAR-T2A-RQR8 fusion polypeptide comprised a CD28 co-stimulatory domain having an amino acid sequence of:

(SEQ ID NO: 1103)
RSKRSRLLHSDYMNMTPRRPGPTRKHYQPYAPPRDFAAYRS

The BCMA-CAR-T2A-RQR8 fusion polypeptide comprised a CD3z signaling domain having an amino acid sequence of:

(SEQ ID NO: 1104)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

The BCMA-CAR-T2A-RQR8 fusion polypeptide comprised a GSG Linker having an amino acid sequence of:

GSG

The BCMA-CAR-T2A-RQR8 fusion polypeptide comprised a T2A peptide having an amino acid sequence of:

(SEQ ID NO: 1106)
EGRGSLLTCGDVEENPGP

The BCMA-CAR-T2A-RQR8 fusion polypeptide comprised a RQR8 protein having an amino acid sequence of:

(SEQ ID NO: 15453)
MGTSLLCWMALCLLGADHADACPYSNPSLCSGGGGSELPTQGTFSNVSTN
VSPAKPTTTACPYSNPSLCSGGGGSPAPRPPTPAPTIASQPLSLRPEACR
PAAGGAVHTRGLDFACDIYIWAPLAGTCGVLLLSLVITLYCNHRNRRRVC
KCPRPVV*

Briefly, the template RNA for use with the Vingi-1_Acar gene modifying system comprises the following sequences from 5′ to 3′: 5′ UTRᵣₑₜᵣₒ, bGH polyA, WPRE, BCMA-CAR-T2A-RQR8, Kozak sequence, MND promoter, 3′ UTRᵣₑₜᵣₒ.

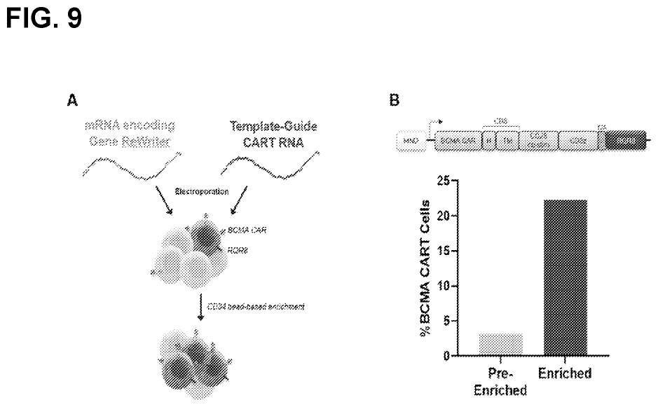

The Vingi-1_Acar gene modifying system and BCMA-CAR-T2A-RQR8 template RNA was introduced into primary human CD3+ T cells (FIG. 9A). Briefly, human CD3+ T cells stored in liquid nitrogen from one donor were thawed in TexMACS culture media (Miltenyi) supplemented with 5% Human AB serum, IL2 (20 IU/mL; Miltenyi), IL7 (10 ng/mL; Miltenyi), and IL15 (5 ng/mL; Miltenyi). Cells were activated immediately post thaw by supplementing the culture media with TransACT (according to manufacturer's protocol) for two days.

Activated T cells were electroporated with mRNA encoding Vingi-1_Acar gene modifying polypeptide and BCMA CAR template RNA. As a control, T cells that underwent no genetic modification were cultured along with cells that underwent the electroporated conditions. For the electroporation, a total of 500-700 million T cells were electroporated with 500 μg/mL of RNA (mass ratio of 2 template:1 driver) in a volume of 400-450 uLs of OptiMEM to maintain a cell concentration of 4e8 cells/mL. Electroporations were quenched in expansion media (TexMACS, 100 IU/mL IL2, 10 ng/mL of IL7, 5 ng/mL of IL15, 5% Human AB serum). Electroporated T cells were expanded until day 6 and Enrichment of the gene edited T cells was performed using the surface expressed RQR8 tag using the MultiMACS magnetic separation system and anti-CD34 magnetic beads (Miltenyi). Post-enrichment, the cells were expanded using expansion media (TexMACS, 100 IU/mL IL2, 10 ng/mL of IL7, 5 ng/mL of IL15, 5% Human AB serum) and analyzed by flow cytometry for CAR expression after 16 days. 100 IU/mL IL2 was also added to the cell culture on these days to promote cell expansion. FIG. 9B (top) shows a schematic of the delivered BCMA CAR and (bottom) shows a graph of the % BCMA CAR positive cells pre-enrichment and post-enrichment. The results demonstrate that cells begin with approximately 2.5% BCMA-CAR and can be enriched up to approximately 22% using the CD34 bead-based methods described herein. Such increases in enrichment can enable use of the cells for further downstream studies or for therapeutic purposes.

On day 9 of the experiment, cells were harvested and function was assessed via BCMA target cell killing assay. Briefly, BCMA-CART cells were co-cultured for 24 hours with BCMA-positive tumor cell lines (H929, BCMA Cell line 1 and RPMI-8226, BCMA Cell line 2) at serially decreasing T cell to tumor ratios. The co-cultures were harvested after 24 hours, and culture supernatants were assessed for IFNγ concentration via ELISA. Cells from co-culture were stained with antibodies to uniquely identify surface markers for both target cells and effector cells. Stained co-cultures were acquired on a flow cytometer with dead cell exclusion via a vital dye. Targets were positively identified during analysis and absolute counts of target cells for each condition were generated by volumetric readout by the flow cytometer. All conditions were normalized to a target only well to control for tumor growth via the following equation: % Killing=1−(number of target cells in test condition/number of target cells in normalization control)*100.

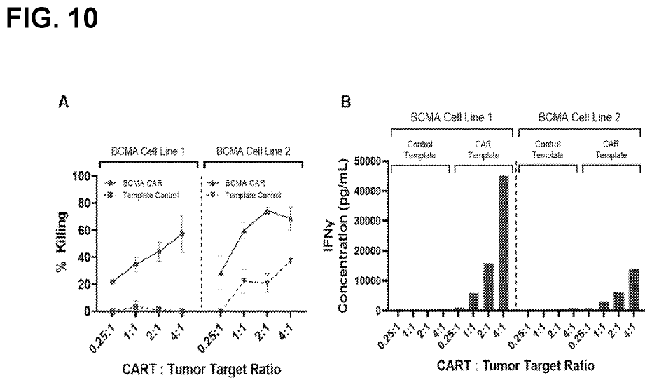

As shown in FIGS. 10A-10B, BCMA-CART cells generated using the Vingi-1_Acar gene modifying system were shown to be functional via a dose dependent reduction in the number of targets observed in each effector/target condition as compared to the normalization control for both target cell lines (FIG. 10A). IFNγ levels from corresponding culture supernatants also show a correlative dose response to observed killing from BCMA CART cells tested (FIG. 10B). These data, taken together, indicate a functional and potent CART product derived from the Vingi-1_Acar gene modifying system that is able to control tumor growth in vitro.

### Example 8: Evaluating Tumor Cell Killing in Cancer-Bearing Mice by BCMA CART Cells Generated Using an Exemplary Retrotransposon-Based Gene Modifying System

This Example demonstrates that exemplary CART cells expressing a BCMA-targeted CAR and generated using a Vingi-1_Acar gene modifying system described herein are capable of killing exemplary human cancer cells (multiple myeloma tumor cells) in mouse models.

BCMA CART cells were generated as described in Example 7 using the Vingi-1_Acar gene modifying system described in Example 7.

NSG mice (NOD.Cg-Prkdcscid Il2rgtm1Wjl/SzJ), 8-10 weeks of age (The Jackson Laboratory) were implanted via subcutaneous injection in the right flank with 1×10⁶ RPMI-8226 human multiple myeloma tumor cells (ATCC). Tumors were measured twice weekly with digital calipers and the tumor volume was calculated using the formula Volume (mm3)=(L×W²)/2 where L=length and W=Width. When tumors reached an average size of ˜30 mm³ (Day 24), mice (n=5/group) were treated with 5×10⁶ anti-BCMA CAR+ T cells (as described previously) or un-transduced (UTD) T cells via intravenous injection. As the percentage of CAR+ T cells was ˜20% of the total T cell count, each mouse received 2.5×10⁷ total T cells, whereas mice in the control groups (n=5/group) received either 2.5×10⁷ un-transduced T cells or PBS vehicle control.

FIG. 11A-11C compares individual animal RPMI-8266 tumor growth kinetics in each of the treatment groups (vehicle treated FIG. 11A, untransduced T cells treated FIG. 11B, and BCMA CART treated FIG. 11C). By Day 39 (15 days post-treatment), 4/5 mice treated with anti-BCMA CAR T cells had cleared their tumors and the tumor that was not yet cleared at that timepoint was substantially reduced in size. In contrast, animals treated with vehicle control experienced continued tumor growth. Likewise, animals treated with UTD T cells experienced continued tumor growth until Day 36 when a presumed anti-tumor allograft rejection response began to impact the experiment, slowing tumors growth in this group. Without wishing to be bound by theory, the allograft rejection response is thought to be an artifact of the model system; the timing of allograft rejection onset varies from one human T cell donor to another. Regardless, the results showed a reduction in tumor volume when mice were treated with BCMA CART cells derived from the gene modifying system described herein, and increases or increases and plateauing of tumor volume in the control treated mice.

These data demonstrate that anti-BCMA CAR T cells generated via retrotransposon derived gene modifying systems, (e.g., a Vingi-1_Acar gene modifying system) are effective at clearing multiple myeloma tumor xenografts in NSG mice.

### Example 9: Evaluating Combined Activity of Exemplary TRAC Human Template RNAs with Gene Modifying Systems to Install CARs

This example describes the use of a first exemplary system containing a first heterologous gene modifying polypeptide and a template RNA to produce an insertion of 5 nucleotides (TAGTG) in the first exon of human TRAC gene in primary human T cells, and an exemplary gene modifying system containing a gene modifying polypeptide and a template RNA to insert expression cassettes for a chimeric antigen receptor (CAR) or a GFP molecule into the genome of the primary human T cells, as well as quantification of said activities. The insertion generated a frameshift that resulted in a non-functional gene (also referred to as a knock-out).

In this example, the template RNA of the heterologous gene modifying system contained:
(1) a gRNA spacer;
(2) a gRNA scaffold;
(3) a heterologous object sequence; and
(4) a primer binding site (PBS) sequence.

The exemplary template RNA generated and used in the heterologous gene modifying system is given in Table E1. Nucleotide modifications are noted as follows: phosphorothioate linkages denoted by an asterisk, 2′-O-methyl groups denoted by an ‘m’ preceding a nucleotide.

TABLE E1
Exemplary Template RNAs and Sequences

| | | | SEQ |
|---|---|---|---|
| | | | ID |
| Name | Descriptor | Sequence | NO |
| RNACS6062 | TRAC5_ | mG*mC*mU*rGrGrUrArCrArCrGrGrCrArGrGrGrU | 15,001 |
| | P9R22_ | rCrArGrUrUrUrUrArGrAmGmCmUmAmGmAmAmAmUm | |
| | ins5-nick | AmGmCrArArGrUrUrArArArArUrArArGrGrCrUrA | |
| | | rGrUrCrCrGrUrUrArUrCrAmAmCmUmUmGmAmAmAm | |
| | | AmAmGmUmGmGmCmAmCmCmGmAmGmUmCmGmGmUmGmC | |
| | | rGrArUrArUrCrCrArGrArArCrCrCrUrGrArUrAr | |
| | | GrUrGrCrCrCrUrGrC*mC*mG*mU | |

Table E1 shows the sequences of E1 without chemical modifications. In some embodiments, the sequences of Table E1A may be used without chemical modifications, or with one or more chemical modifications.

TABLE E1A
Table E1 Sequences without Chemical Modifications

| | | | SEQ |
|---|---|---|---|
| | | | ID |
| RNACS | Name | Sequence | NO |
| RNACS6062 | TRAC5_ | GCUGGUACACGGCAGGGUCAGUUUUAGAGCUAGAAAUAGCA | 15,000 |
| | P9R22_ | AGUUAAAAUAAGGCUAGUCCGUUAUCAACUUGAAAAAGUGG | |
| | ins5- | CACCGAGUCGGUGCGAUAUCCAGAACCCUGAUAGUGCCCUG | |
| | nick | CCGU | |

In this example, exemplary heterologous gene modifying polypeptides of the first gene modifying system contained:
(1) an endonuclease and/or DNA binding domain;
(2) a peptide linker; and
(3) a reverse transcriptase (RT) domain,
   where (1), (2), and (3) are heterologous to each other.
   The exemplary heterologous modifying polypeptide of the heterologous gene modifying system was RNAIVT593, and comprised the amino acid sequence of indicated in Table E2. In some embodiments, heterologous gene modifying polypeptides contain one or more nuclear localization sequences (NLSs). The gene modifying polypeptide of RNAIVT593 comprises an N-terminal NLS and a C-terminal NLS.

TABLE E2
Exemplary heterologous gene modifying polypeptide and Sequence

| | | SEQ |
|---|---|---|
| Name | AA sequence | ID NO |
| RNAIVT593 | MPAAKRVKLDGGDKKYSIGLDIGTNSVGWAVITDEYKVPSK | 15008 |
| | KFKVLGNTDRHSIKKNLIGALLFDSGETAEATRLKRTARRRY | |
| | TRRKNRICYLQEIFSNEMAKVDDSFFHRLEESFLVEEDKKHER | |
| | HPIFGNIVDEVAYHEKYPTIYHLRKKLVDSTDKADLRLIYLAL | |
| | AHMIKFRGHFLIEGDLNPDNSDVDKLFIQLVQTYNQLFEENPI | |
| | NASGVDAKAILSARLSKSRRLENLIAQLPGEKKNGLFGNLIAL | |
| | SLGLTPNFKSNFDLAEDAKLQLSKDTYDDDLDNLLAQIGDQY | |
| | ADLFLAAKNLSDAILLSDILRVNTEITKAPLSASMIKRYDEHH | |
| | QDLTLLKALVRQQLPEKYKEIFFDQSKNGYAGYIDGGASQEE | |
| | FYKFIKPILEKMDGTEELLVKLNREDLLRKQRTFDNGSIPHQI | |
| | HLGELHAILRRQEDFYPFLKDNREKIEKILTFRIPYYVGPLARG | |
| | NSRFAWMTRKSEETITPWNFEEVVDKGASAQSFIERMTNFDK | |
| | NLPNEKVLPKHSLLYEYFTVYNELTKVKYVTEGMRKPAFLSG | |
| | EQKKAIVDLLFKTNRKVTVKQLKEDYFKKIECFDSVEISGVED | |
| | RFNASLGTYHDLLKIIKDKDFLDNEENEDILEDIVLTLTLFEDR | |
| | EMIEERLKTYAHLFDDKVMKQLKRRRYTGWGRLSRKLINGI | |
| | RDKQSGKTILDFLKSDGFANRNFMQLIHDDSLTFKEDIQKAQ | |
| | VSGQGDSLHEHIANLAGSPAIKKGILQTVKVVDELVKVMGRH | |
| | KPENIVIEMARENQTTQKGQKNSRERMKRIEEGIKELGSQILK | |
| | EHPVENTQLQNEKLYLYYLQNGRDMYVDQELDINRLSDYDV | |
| | DHIVPQSFLKDDSIDNKVLTRSDKARGKSDNVPSEEVVKKMK | |
| | NYWRQLLNAKLITQRKFDNLTKAERGGLSELDKAGFIKRQLV | |
| | ETRQITKHVAQILDSRMNTKYDENDKLIREVKVITLKSKLVSD | |
| | FRKDFQFYKVREINNYHHAHDAYLNAVVGTALIKKYPKLESE | |
| | FVYGDYKVYDVRKMIAKSEQEIGKATAKYFFYSNIMNFFKTE | |
| | ITLANGEIRKRPLIETNGETGEIVWDKGRDFATVRKVLSMPQV | |
| | NIVKKTEVQTGGFSKESILPKRNSDKLIARKKDWDPKKYGGF | |
| | DSPTVAYSVLVVAKVEKGKSKKLKSVKELLGITIMERSSFEK | |
| | NPIDFLEAKGYKEVKKDLIIKLPKYSLFELENGRKRMLASAGE | |
| | LQKGNELALPSKYVNFLYLASHYEKLKGSPEDNEQKQLFVEQ | |
| | HKHYLDEIIEQISEFSKRVILADANLDKVLSAYNKHRDKPIRE | |
| | QAENIIHLFTLTNLGAPAAFKYFDTTIDRKRYTSTKEVLDATLI | |
| | HQSITGLYETRIDLSQLGGDSGGSSGGSSGSETPGTSESATPES | |
| | SGGSSGGSSTLNIEDEYRLHETSKEPDVSLGSTWLSDFPQAWA | |
| | ETGGMGLAVRQAPLIIPLKATSTPVSIKQYPMSQEARLGIKPHI | |
| | QRLLDQGILVPCQSPWNTPLLPVKKPGTNDYRPVQDLREVNK | |
| | RVEDIHPTVPNPYNLLSGLPPSHQWYTVLDLKDAFFCLRLHPT | |
| | SQPLFAFEWRDPEMGISGQLTWTRLPQGFKNSPTLFNEALHR | |
| | DLADFRIQHPDLILLQYVDDLLLAATSELDCQQGTRALLQTL | |
| | GNLGYRASAKKAQICQKQVKYLGYLLKEGQRWLTEARKET | |
| | VMGQPTPKTPRQLREFLGKAGFCRLFIPGFAEMAAPLYPLTKP | |
| | GTLFNWGPDQQKAYQEIKQALLTAPALGLPDLTKPFELFVDE | |
| | KQGYAKGVLTQKLGPWRRPVAYLSKKLDPVAAGWPPCLRM | |
| | VAAIAVLTKDAGKLTMGQPLVILAPHAVEALVKQPPDRWLS | |
| | NARMTHYQALLLDTDRVQFGPVVALNPATLLPLPEEGLQHN | |
| | CLDILAEAHGTRPDLTDQPLPDADHTWYTDGSSLLQEGQRKA | |
| | GAAVTTETEVIWAKALPAGTSAQRAELIALTQALKMAEGKK | |
| | LNVYTDSRYAFATAHIHGEIYRRRGWLTSEGKEIKNKDEILAL | |
| | LKALFLPKRLSIIHCPGHQKGHSAEARGNRMADQAARKAAIT | |
| | ETPDTSTLLIENSSPSGGSKRTADGSEFEKRTADGSEFESPKKK | |
| | AKVE | |

In this example, exemplary gene modifying polypeptides of the gene modifying system contained:
(1) an endonuclease and/or DNA binding domain; and
(2) a reverse transcriptase (RT) domain,
   where (1) and (2) are both derived from a retrotransposon (and in this Example, from the same retrotransposons). Accordingly, the gene modifying polypeptides in this Example were examples of retrotransposon gene modifying polypeptides.

The gene modifying system used in this Example comprised either of two exemplary retrotransposon gene modifying polypeptides: RNAIVT696 (SEQ ID NO: 15,002) and RNAIVT1123 (SEQ ID NO:15,003), and amino acid sequences are given in Table E4. In some embodiments, gene modifying polypeptides contain one or more NLSs and/or one or more tag sequences (e.g., to facilitate purification, detection, or quantification). The gene modifying polypeptide of RNAIVT696 comprises a C-terminal NLS and Hibit tag. The gene modifying polypeptide of RNAIVT1123 comprises a C-terminal NLS and Hibit tag.

TABLE E4
Exemplary gene modifying polypeptide and Sequences

| | | SEQ |
|---|---|---|
| Name | AA sequence | ID NO |
| RNAIVT696 | MDEYQRSLSRPLLTIMSINIEGLSLAKEELLAKMSEDISCDILCI | 15,002 |
| | QETHRDITMRRPKILGMQLAVERPHRQYGSAIFVRSGVAISAT | |
| | SLTEVNNIEILSVELDSCTVSSLYKPPGADFYFTPPTSCHNHEA | |
| | HFVVGDFNSHSCVWGYDEDDRNGEAVLTWADNSRMSLLHD | |
| | SKLPPSFNSGRWKRGYNPDLIFVKESISHQCTKRVLNPIPNTQ | |
| | HRPICCVAYAAVRPKSVPFRRRYNFNKANWTKFTETLEAAIS | |
| | DIEPSIENYDLFVEAVKRSSRLSIPRGCRTSYLPGLNEESLNQL | |
| | QEYLRLFQENPYSDGTIAAGQKLSTALANAKKDRWIELLENL | |
| | DMSKSSRKAWQLLRRLDSDPLVNPGHANVTPDQIAHQLIQN | |
| | GKTNCSRIKMKINRVPELETHQLSSPLNLKELREAIKRCKTGK | |
| | APGLDDLMMEQIKHLGAKAENWLLKFYNQCLAHKQIPRAW | |
| | RKTKIIAILKPGKDASNARNYRPISLLCHLYKVYERMLLNRLG | |
| | PVIEPKLIAQQAGFRPGKNCTGQILHLTEHIEEGYEKGCITGTV | |
| | FVDLTAAYDTVQHRKMLHKVYHITRDFDFTKTVQTLLENRS | |
| | FYVEFQGQKSRWRRQKNGLPQGSVLAPTLFNIFTNDQPQPPL | |
| | TKSFIYADDLGLTTQAKDFETVEKQLTNALKDLSSYYKENHL | |
| | KPNPAKTQVCAFHLRNREANRKLKVTWEGQELEHCFHPKYL | |
| | GVTLDRTLTYRKHCMNTKHKVAARNNILRKLTGSAWGADP | |
| | QVIRTSALALSFSTAEYACPVWHKSAHAKQVDIALNETCRIIT | |
| | GCLKPTPVDKLYKLAGIAPPDVRREVAANGERKKVEHCESHP | |
| | LHGYHPPPTRLKSRKGFMRTTTPLDVPPAAARVSLWAAKPG | |
| | NSNWMAPQEGLPPGANQEWATWKSLNRLRSGVGRSKDNLA | |
| | RWHYLEESSTLCDCGAEQTTQHMYACPQCPASCTEEELFKAT | |
| | DNAVAVARFWSKTIGGGSPKKKRKVSGSETPGTSESATPESV | |
| | SGWRLFKKIS | |
| RNAIVT1123 | MDSTAHPNQGRGLEKVSQTLPALQTPGQHTAAGGSSPLSGR | 15,003 |
| | NQRKNTKKLLLGAWNIRTLLDRENTPRPERRTALIGKELARY | |
| | NIDIAALSETRLPEEGSLSEPTTGYTFFWKGRASNEDRIHGVG | |
| | LAIKTSLLKQLPDLPVGISERLMKIRLPLSKDRYATIISAYAPTL | |
| | TSTEETIEQFYSDLSAVLHSVPTNDKLILLGDFNARVGQDHER | |
| | WKGVLGKHGVGKMNNNGLLLLSKCSEFELTITNTVFRMANK | |
| | YKTTWMHPRSKQWHLIDYIIVRRRDIQDVKITRAMRGAECW | |
| | TDHRLVRATLQMRIAPRHPKRAQTVRAFYNVSRLRDPSYLQT | |
| | FQSCLDDKLSAKGPLTGSSTEKWNQFRDAVKETSKAVLGPK | |
| | QRNHQDWFDENNTAIEDLLSKKNKAFMEWQNNPNSAPKKD | |
| | RFKSLQATAQREIRKMQDRWWEKKAEEIQRFADMKNYKQFF | |
| | SALKTVYGPLKPTTTPLLSSDGDTLIKDKKGISNRWKEHFSQL | |
| | LNRPSSVDQSALDQIPQNRTIEQLDVPPSIEEVQKAIKQMSAG | |
| | KAPGKDGIPTEVYKALNGKALQAFHIVLTSIWEEEDMPPELR | |
| | DASIVALYKNKGSRAACDNYRGISLLSTAGKILARVILNRLLS | |
| | SVSEQNLPESQCGFRPDRSTIDMVFTVRQMQEKCLEQNLSLYI | |
| | VFIDLTKAFDTVNRDALWVILSKLGCPAKFVKLIQLFHVDMT | |
| | GEVLSGGETSDRFNISNGVKQGCVLAPVLFNLFFTQVLRHAV | |
| | MDLDLGVYIKYRLDGSLFDLRRLTAKTKTTERLILEALFADD | |
| | CALMAHQENHLQTIVDRFSTATKLFGLTISLSKTEVLFQPAPG | |
| | RPTNQPCITIDGTQLSNVNTFKYLGSTIANDGSLDHEINARIQK | |
| | ASQALGRLRCKVLQHRGVSTATKLKVYNAVVLSSLLYGCET | |
| | WTLYRKHMKQLEQFHQRSLRSIMRIRWQDRITNQEVLDRAN | |
| | STSIEVMVLKTQLRWSGHVIRMDPQRIPRQVFYGELSAGLRK | |
| | QGRPKKRFKDQLKSNLKWAGITPKQLELAASDRSSWRTHINH | |
| | AATTFEDERRRRLAAARERRHQATTAPPVTTGVPCPMCHKL | |
| | CASAFGLQSHMRVHRRSGSETPGTSESATPESVSGWRLFKKIS | |

Exemplary templates RNAs comprising sequences capable of binding the exemplary retrotransposon gene modifying polypeptides included RNAIVT354 and RNAIVT381 for RNAIVT696 gene modifying polypeptide and RNAIVT610 and RNAIVT1200 for RNAIVT1123 gene modifying polypeptide. Sequences are given in Table E5.

TABLE E5
Exemplary gene modifying RNA template and Sequences

| | | SEQ ID |
|---|---|---|
| Name | RNA sequence (5′ to 3′) | NO |
| RNAIVT354 | GGGGGACACGGAAAGAGCCUCCCCGAAGAUUGAGUGAAUUCAGUCGGGCGUCCCCUGGGCAACGUU | 15454 |
| | UCUUGUAAGCGGCCGAUCUUUCCACCCCAAAAGCAUUGGAUGAGGAUCCGUUCUUUCCGCCUCAGA | |
| | AGCCAUAGAGCCCACCGCAUCCCCAGCAUGCCUGCUAUUGUCUUCCCAAUCCUCCCCCUUGCUGUCC | |
| | UGCCCCACCCCACCCCCCAGAAUAGAAUGACACCUACUCAGACAAUGCGAUGCAAUUUCCUCAUUUU | |
| | AUUAGGAAAGGACAGUGGGAGUGGCACCUUCCAGGGUCAAGGAAGGCACGGGGGAGGGGCAAACAA | |
| | CAGAUGGCUGGCAACUAGAAGGCACAGUCGAGGCUGAUCAGCGAGUCGACGGUUUACUUGUACAGC | |
| | UCGUCCAUGCCGAGAGUGAUCCCGGCGGCGGUCACGAACUCCAGCAGGACCAUGUGAUCGCGCUUC | |
| | UCGUUGGGGUCUUUGCUCAGGGCGGACUGGGUGCUCAGGUAGUGGUUGUCGGGCAGCAGCACGGGG | |
| | CCGUCGCCGAUGGGGGUGUUCUGCUGGUAGUGGUCGGCGAGCUGCACGCUGCCGUCCUCGAUGUUG | |
| | UGGCGGAUCUUGAAGUUCACCUUGAUGCCGUUCUUCUGCUUGUCGGCCAUGAUAUAGACGUUGUGG | |
| | CUGUUGUAGUUGUACUCCAGCUUGUGCCCCAGGAUGUUGCCGUCCUCCUUGAAGUCGAUGCCCUUC | |
| | AGCUCGAUGCGGUUCACCAGGGUGUCGCCCUCGAACUUCACCUCGGCGCGGGUCUUGUAGUUGCCG | |
| | UCGUCCUUGAAGAAGAUGGUGCGCUCCUGGACGUAGCCUUCGGGCAUGGCGGACUUGAAGAAGUCG | |
| | UGCUGCUUCAUGUGGUCGGGGUAGCGGCUGAAGCACUGCACGCCGUAGGUCAGGGUGGUCACGAGG | |
| | GUGGGCCAGGGCACGGGCAGCUUGCCGGUGGUGCAGAUGAACUUCAGGGUCAGCUUGCCGUAGGUG | |
| | GCAUCGCCCUCGCCCUCGCCGGACACGCUGAACUUGUGGCCGUUUACGUCGCCGUCCAGCUCGACCA | |
| | GGAUGGGCACCACCCCGGUGAACAGCUCCUCGCCCUUGCUCACCAUGGUGGCUUUACCAACAGUACC | |
| | GGAAUGCCAAGCUUGGGUCcUGGGGAGAGAGGUCGGUGAUUCGGUCAACGAGGGAGCCGACUGCCG | |
| | ACGUGCGCUCCGGAGGCUUGCAGAAUGCGGAACACCGCGCGGGCAGGAACAGGGCCCACACUACCG | |
| | CCCCACACCCCGCCUCCCGCACCGCCCCUUCCCGGCCGCUGCUCUCGGCGCGCCCUGCUGAGCAGCCG | |
| | CUAUUGGCCACAGCCCAUCGCGGUCGGCGCGCUGCCAUUGCUCCCUGGCGCUGUCCGUCUGCGAGGG | |
| | UACUAGUGAGACGUGCGGCUUCCGUUUGUCACGUCCGGCACGCCGCGAACCGCAAGGAACCUUCCC | |
| | GACUUAGGGGCGGAGCAGGAAGCGUCGCCGGGGGGCCCACAAGGGUAGCGGCGAAGAUCCGGGUGA | |
| | CGCUGCGAACGGACGUGAAGAAUGUGCGAGACCCAGGGUCGGCGCCGCUGCGUUUCCCGGAACCAC | |
| | GCCCAGAGCAGCCGCGUCCCUGCGCAAACCCAGGGCUGCCUUGGAAAAGGCGCAACCCCAACCCCGC | |
| | GGCCGCUAGUUGCUUGUGAUUUCUUUUCUUUUUUAUUUUAUUUCCAUUAUUUGAAAUGUAUUUGU | |
| | UGUAGCAAUGCUUUUGACACGAAAUAAAUAAA | |
| RNAIVT381 | GGGGGACACGGAAAGAGCCUCCCCGAAGAUUGAGUGAAUUCAGUCGGGCGUCCCCUGGGCAACGUU | 15009 |
| | UCUUGUAAGCGGCCGAUCUUUCCACCCCAAAAGCAUUGGAUGAGUUUACGGAUCCGAAUUCUCGAC | |
| | GGAUCGAUCCGAACAAACGACCCAACACCCGUGCGUUUUAUUCUGUCUUUUUAUUGCCGAUCCCCC | |
| | GGCCGCUUUACUUGUACAGCUCGUCCAUGCCGAGAGUGAUCCCGGCGGCGGUCACGAACUCCAGCA | |
| | GGACCAUGUGAUCGCGCUUCUCGUUGGGGUCUUUGCUCAGGGCGGACUGGGUGCUCAGGUAGUGGU | |
| | UGUCGGGCAGCAGCACGGGGCCGUCGCCGAUGGGGGUGUUCUGCUGGUAGUGGUCGGCGAGCUGCA | |
| | CGCUGCCGUCCUCGAUGUUGUGGCGGAUCUUGAAGUUCACCUUGAUGCCGUUCUUCUGCUUGUCGG | |
| | CCAUGAUAUAGACGUUGUGGCUGUUGUAGUUGUACUCCAGCUUGUGCCCCAGGAUGUUGCCGUCCU | |
| | CCUUGAAGUCGAUGCCCUUCAGCUCGAUGCGGUUCACCAGGGUGUCGCCCUCGAACUUCACCUCGGC | |
| | GCGGGUCUUGUAGUUGCCGUCGUCCUUGAAGAAGAUGGUGCGCUCCUGGACGUAGCCUUCGGGCAU | |
| | GGCGGACUUGAAGAAGUCGUGCUGCUUCAUGUGGUCGGGGUAGCGGCUGAAGCACUGCACGCCGUA | |
| | GGUCAGGGUGGUCACGAGGGUGGGCCAGGGCACGGGCAGCUUGCCGGUGGUGCAGAUGAACUUCAG | |
| | GGUCAGCUUGCCGUAGGUGGCAUCGCCCUCGCCCUCGCCGGACACGCUGAACUUGUGGCCGUUUAC | |
| | GUCGCCGUCCAGCUCGACCAGGAUGGGCACCACCCCGGUGAACAGCUCCUCGCCCUUGCUCACCAUG | |
| | GUGGCUUUACCAACAGUACCGGAAUGCCAAGCUUGGGUCCUGUGUUCUGGCGGCAAACCCGUUGCG | |
| | AAAAAGAACGUUCACGGCGACUACUGCACUUAUAUACGGUUCUCCCCCACCCUCGGGAAAAAGGCG | |
| | GAGCCAGUACACGACAUCACUUUCCCAGUUUACCCCGCGCCACCUUCUCUAGGCACCGGAUCAAUUG | |
| | CCGACCCCUCCCCCCAACUUCUCGGGGACUGUGGGCGAUGUGCGCUCUGCCCUAGUUGCUUGUGAUU | |
| | UCUUUUCUUUUUUAUUUUAUUUCCAUUAUUUGAAAUGUAUUUGUUGUAGCAAUGCUUUUGACACG | |
| | AAAUAAAUAAA | |
| RNAIVT610 | GGGUGUAUGGGGUGCUCAGGGAGGGGUAGUAUCUCUGGUAUGGAGGGCUUGUCGUGCCCUCCUAGG | 15010 |
| | GCAGCUCUCCAGCCUCUGACCCCCACCUGACACCCAGCUCUCACUUGUGGCUCCCAGUAGCUGCUAG | |
| | CAUGUGGCAGCGGCCACACCCCGGGCAACGGCUUCGACAGGCCGGCUAAACCUUGUGAGGGUAGCC | |
| | AUCGGGUCAUCGACCCCUGGUGAACCAGGGCUUUGCUCACCCAGCAUGUGAAGACUGCUUCGGCUG | |
| | AACAGACGGAAGAAACCAAUAAGAAGGUUCAACGGCUGAGAGGGCGACGCAGCAAAGCACUGUGGA | |
| | GUGCUUAGGGCGUGUUGGAGCACAAAGGACAACACGGCCAUCCAAUGCAGCUGAGGAAGUCUCCAG | |
| | AUGUAACAAUUUUUCGUGCCACUGGACCCAGGCUUCCAACGCCGAGAGAGUGGGACUGUCUCUGUG | |
| | CAUCGGCUUUUCCACUUAAAUCUCUUUCACGCACAAGUAUCUUUGUGCACACUCAUCUAUCCUAAC | |
| | CCCGUCCACCCUCUUCAAGACCUGCGGCGAUGGGGGAGUGGCGACGCAACAGGUGGAGGUGACCAC | |
| | UGGCAGUUGUAGUCACGAUCCUGCACGUAGGCGGCCCACGGACCAGUGGUCGCUCGGCCCUGUGGG | |
| | CAGCAGGGACGUUCGGCAGCAUCCUGGGCGACUGAGCAGCCCUCUCUAGGUUUACGGAUCCGAAUU | |
| | CUCGACGGAUCGAUCCGAACAAACGACCCAACACCCGUGCGUUUUAUUCUGUCUUUUUAUUGCCGA | |
| | UCCCCCGGCCGCUUUACUUGUACAGCUCGUCCAUGCCGAGAGUGAUCCCGGCGGCGGUCACGAACUC | |
| | CAGCAGGACCAUGUGAUCGCGCUUCUCGUUGGGGUCUUUGCUCAGGGCGGACUGGGUGCUCAGGUA | |
| | GUGGUUGUCGGGCAGCAGCACGGGGCCGUCGCCGAUGGGGGUGUUCUGCUGGUAGUGGUCGGCGAG | |
| | CUGCACGCUGCCGUCCUCGAUGUUGUGGCGGAUCUUGAAGUUCACCUUGAUGCCGUUCUUCUGCUU | |
| | GUCGGCCAUGAUAUAGACGUUGUGGCUGUUGUAGUUGUACUCCAGCUUGUGCCCCAGGAUGUUGCC | |
| | GUCCUCCUUGAAGUCGAUGCCCUUCAGCUCGAUGCGGUUCACCAGGGUGUCGCCCUCGAACUUCACC | |
| | UCGGCGCGGGUCUUGUAGUUGCCGUCGUCCUUGAAGAAGAUGGUGCGCUCCUGGACGUAGCCUUCG | |
| | GGCAUGGCGGACUUGAAGAAGUCGUGCUGCUUCAUGUGGUCGGGGUAGCGGCUGAAGCACUGCACG | |
| | CCGUAGGUCAGGGUGGUCACGAGGGUGGGCCAGGGCACGGGCAGCUUGCCGGUGGUGCAGAUGAAC | |
| | UUCAGGGUCAGCUUGCCGUAGGUGGCAUCGCCCUCGCCCUCGCCGGACACGCUGAACUUGUGGCCG | |
| | UUUACGUCGCCGUCCAGCUCGACCAGGAUGGGCACCACCCCGGUGAACAGCUCCUCGCCCUUGCUCA | |
| | CCAUGGUGGCUUUACCAACAGUACCGGAAUGCCAAGCUUGGGUCCUGUGUUCUGGCGGCAAACCCG | |
| | UUGCGAAAAAGAACGUUCACGGCGACUACUGCACUUAUAUACGGUUCUCCCCCACCCUCGGGAAAA | |
| | AGGCGGAGCCAGUACACGACAUCACUUUCCCAGUUUACCCCGCGCCACCUUCUCUAGGCACCGGAUC | |
| | AAUUGCCGACCCCUCCCCCCAACUUCUCGGGGACUGUGGGCGAUGUGCGCUCUGCCCUGAAACUGCA | |
| | CAAAGACAAUAGUCAUUCUCGAUCACCGAGAGACUACCACU | |
| RNAIVT1200 | GGGUGUAUGGGGUGCUCAGGGAGGGGUAGUAUCUCUGGUAUGGAGGGCUUGUCGUGCCCUCCUAGG | 15011 |
| | GCAGCUCUCCAGCCUCUGACCCCCACCUGACACCCAGCUCUCACUUGUGGCUCCCAGUAGCUGCUAG | |
| | CAUGUGGCAGCGGCCACACCCCGGGCAACGGCUUCGACAGGCCGGCUAAACCUUGUGAGGGUAGCC | |
| | AUCGGGUCAUCGACCCCUGGUGAACCAGGGCUUUGCUCACCCAGCAUGUGAAGACUGCUUCGGCUG | |
| | AACAGACGGAAGAAACCAAUAAGAAGGUUCAACGGCUGAGAGGGCGACGCAGCAAAGCACUGUGGA | |
| | GUGCUUAGGGCGUGUUGGAGCACAAAGGACAACACGGCCAUCCAAUGCAGCUGAGGAAGUCUCCAG | |
| | AUGUAACAAUUUUUCGUGCCACUGGACCCAGGCUUCCAACGCCGAGAGAGUGGGACUGUCUCUGUG | |
| | CAUCGGCUUUUCCACUUAAAUCUCUUUCACGCACAAGUAUCUUUGUGCACACUCAUCUAUCCUAAC | |
| | CCCGUCCACCCUCUUCAAGACCUGCGGCGAUGGGGGAGUGGCGACGCAACAGGUGGAGGUGACCAC | |
| | UGGCAGUUGUAGUCACGAUCCUGCACGUAGGCGGCCCACGGACCAGUGGUCGCUCGGCCCUGUGGG | |
| | CAGCAGGGACGUUCGGCAGCAUCCUGGGCGACUGAGCAGCCCUCUCUAGGUUUACGGAUCCGAAUU | |
| | CUCGACGGAUCGAUCCGAACAAACGACCCAACACCCGUGCGUUUUAUUCUGUCUUUUUAUUGCCGA | |
| | UCCCCCGGCCGCUUUAGCGGGGUGGCAAGGCUUGCAUAUGCAACGCAUCAUAAGUGUCCUUGGUGG | |
| | CUGUAGAGAGACCCUGGUACAGACCGUCAUGCCCCUUGCCUCUGCGUCUUUCGCCUUUCAUUCCGA | |
| | UCUCGCUAUAGGCUUCAGCCAUCUUGUCCUUCUGCAGCUCAUUGUACAACCCCUCCUGAGGAUUUU | |
| | UUCUCCGGGGCUUUCCCCCCAUCUCGGGAUCCCUGCCCCGUCUCUUGUCCAGAACAUCGUACUCUUC | |
| | UCGCCUGCCGAGAUUCAAUUCAUUGUAGAGCUGAUUCUGACCCUGCUGAUAUGCGGGAGCAUCGGC | |
| | GCUUCUAGAAAAUUUCACCCUGGAGCGAUAGGCUGCGAAGUCGCGUGGUGGGGCAUAGGGCUGGUA | |
| | AUGCUUGCGGGUGGGCCCGGGGCGGCGGGGAGUCAUGUUCAUGUAGUCACUGUGCAGGAGCCUGCU | |
| | CCUCUUACUCCUACAAUACAGAGUGAUCACAAGGGAGAGAAGCAACACCCCACAUGUUCCGGCCAA | |
| | AGGGGCCCAAAUAUAAAUAUCACACGCGAAGUCCAGGCCUCGCGUAUGGACGGCGCCUCCUGCUGC | |
| | GGGCCUGCAAGCUUCAGGUCUGAGGCUCAAUGGUUGGCUGGCUAUAGUUGGAGCGGGUGUGGGGGG | |
| | CCUUGGAGCAGGUGUUGUGGUCUUGAUCUCCAGUUUGGUCCCCUGACCGAAAGUCCAGGGGACGUG | |
| | ACUGGUCUCUGCACAAUAGUAGACGCCGACGUCCUCAGCCUCGACCCGACUAAUCUUCAGGGUGAA | |
| | AUCGGCACCGCUACCUGAGCCGGAAAAGCGAUCCGGGACCCCGCUGAAACGGUUGGAAACCUUGUA | |
| | GAUCAGGAGCUGUGGACUUUGGCCCGGCUUCUGCAGAUACCAGUGCAGGUAGGUGUUGCCGUUAGA | |
| | GUGGACCAGACUCUGGGAACUCUUGCAGGAGAUGCUCGCCGGCUCACCAGGGGUCACAGACAGAGA | |
| | CAGUGGAGUCUGGGUCAUCACGAUAUCGCUGCCACCCCCUCCAGAGCCACCUCCGCCGGAGCCACCG | |
| | CCACCGGAAGACACUGUCACCAUGGUGCCCUGGCCCCAGACGUCGAAGUACCAGUCAUAAUCAUAC | |
| | AGGGAGGCGCAGAAAUACACGGCGGUAUCUUCGGACCUGAGACUAGACAGCUCCAUGUAUGCCGUA | |
| | GAACUUGACGUGUCGCGGGUCAUGGUAACCCUGCCGGUGAACUUCUGGUUGUACUCAGAGUUUCCG | |
| | CUCGCAAAAUAGAUCCAACCCAUCCACUCGAGUCCCUGCCCGGGAGCCUGCCGCACCCAAUUGAUAU | |
| | AGUAAUCUGGGAAGCUGUAGCCGGAAGCCUUACAGCUCACUUUCACGCUGGCGCCAGGCUUUUUUA | |
| | CCUCCGCGCCGGACUGAACAAGCUGCACCUGCGGGCGAGCGGCAUGCAAGAGCAGGGCAAGAGGCA | |
| | GAAGCAGGGCGGUCACCGGCAGAGCCAUGGUGGCUUUACCAACAGUACCGGAAUGCCAAGCUUGGG | |
| | UCUGGGCUCUUUUAUUGAGCUCGGGGAGCAGAAGCGCGCGAACAGAAGCGAGAAGCGAACUGAUUG | |
| | GUUAGUUCAAAUAAGGCACAGGGUCAUUUCAGGUCCUUGGGGCACCCUGGAAACAUCUGAUGGUUC | |
| | UCUAGAAACUGCUGAGGGCGGGACCGCAUCUGGGGACCAUCUGUUCUUGGCCCUGAGCCGGGGCAG | |
| | GAACUGCUUACCACAGAUAUCCUGUUUGGCCCAUAUUCUGCUGUUCCAACUGUUCUUGGCCCUGAG | |
| | CCGGGGCAGGAACUGCUUACCACAGAUAUCCUGUUUGGCCCAUAUUCUGCUGUCUCUCUGUUCCUA | |
| | ACCUUGAUCCUAGCUUGCCAAACCUACAGGUGGGGUCUUUCAUUCCCCCCUUUUUCUGGAGACUAA | |
| | AUAAAUGAAACUGCACAAAGACAAUAGUCAUUCUCGAUCACCGAGAGACUACCACU | |

TABLE E6
Exemplary UTRᵣₑₜᵣₒ Sequences Used in the Template Sequences of Table E5

| Template | 5′UTRᵣₑₜᵣₒ | 3′UTRᵣₑₜᵣₒ |
|---|---|---|
| RNAIVT354 | GGGGGACACGGAAAGAGCCTCCCCGAAGATTGAGTGAATTCAGTCGGG | TAGTTGCTTGTGATTTCTTTTCTTTTTTAT |
| | CGTCCCCTGGGCAACGTTTCTTGTAAGCGGCCGATCTTTCCACCCCAAA | TTTATTTCCATTATTTGAAATGTATTTGTT |
| | AGCATTGGATGA (SEQ ID NO: 704) | GTAGCAATGCTTTTGACACGAAATAAAT |
| | | AAA (SEQ ID NO: 15455) |
| NAIVT3 | GGGGGACACGGAAAGAGCCTCCCCGAAGATTGAGTGAATTCAGTCGGG | TAGTTGCTTGTGATTTCTTTTCTTTTTTAT |
| | CGTCCCCTGGGCAACGTTTCTTGTAAGCGGCCGATCTTTCCACCCCAAA | TTTATTTCCATTATTTGAAATGTATTTGTT |
| | AGCATTGGATGA (SEQ ID NO: 704) | GTAGCAATGCTTTTGACACGAAATAAAT |
| | | AAA (SEQ ID NO: 15455) |
| RNAIVT610 | GGGTGTATGGGGTGCTCAGGGAGGGGTAGTATCTCTGGTATGGAGGGC | TGAAACTGCACAAAGACAATAGTCATTC |
| | TTGTCGTGCCCTCCTAGGGCAGCTCTCCAGCCTCTGACCCCCACCTGAC | TCGATCACCGAGAGACTACCACT (SEQ ID |
| | ACCCAGCTCTCACTTGTGGCTCCCAGTAGCTGCTAGCATGTGGCAGCGG | NO: 802) |
| | CCACACCCCGGGCAACGGCTTCGACAGGCCGGCTAAACCTTGTGAGGG | |
| | TAGCCATCGGGTCATCGACCCCTGGTGAACCAGGGCTTTGCTCACCCAG | |
| | CATGTGAAGACTGCTTCGGCTGAACAGACGGAAGAAACCAATAAGAAG | |
| | GTTCAACGGCTGAGAGGGCGACGCAGCAAAGCACTGTGGAGTGCTTAG | |
| | GGCGTGTTGGAGCACAAAGGACAACACGGCCATCCAATGCAGCTGAGG | |
| | AAGTCTCCAGATGTAACAATTTTTCGTGCCACTGGACCCAGGCTTCCAA | |
| | CGCCGAGAGAGTGGGACTGTCTCTGTGCATCGGCTTTTCCACTTAAATC | |
| | TCTTTCACGCACAAGTATCTTTGTGCACACTCATCTATCCTAACCCCGTC | |
| | CACCCTCTTCAAGACCTGCGGCGATGGGGGAGTGGCGACGCAACAGGT | |
| | GGAGGTGACCACTGGCAGTTGTAGTCACGATCCTGCACGTAGGCGGCC | |
| | CACGGACCAGTGGTCGCTCGGCCCTGTGGGCAGCAGGGACGTTCGGCA | |
| | GCATCCTGGGCGACTGAGCAGCCCTCTCTAG (SEQ ID NO: 702) | |
| RNAIVT1200 | GGGTGTATGGGGTGCTCAGGGAGGGGTAGTATCTCTGGTATGGAGGGC | TGAAACTGCACAAAGACAATAGTCATTC |
| | TTGTCGTGCCCTCCTAGGGCAGCTCTCCAGCCTCTGACCCCCACCTGAC | TCGATCACCGAGAGACTACCACT (SEQ ID |
| | ACCCAGCTCTCACTTGTGGCTCCCAGTAGCTGCTAGCATGTGGCAGCGG | NO: 802) |
| | CCACACCCCGGGCAACGGCTTCGACAGGCCGGCTAAACCTTGTGAGGG | |
| | TAGCCATCGGGTCATCGACCCCTGGTGAACCAGGGCTTTGCTCACCCAG | |
| | CATGTGAAGACTGCTTCGGCTGAACAGACGGAAGAAACCAATAAGAAG | |
| | GTTCAACGGCTGAGAGGGCGACGCAGCAAAGCACTGTGGAGTGCTTAG | |
| | GGCGTGTTGGAGCACAAAGGACAACACGGCCATCCAATGCAGCTGAGG | |
| | AAGTCTCCAGATGTAACAATTTTTCGTGCCACTGGACCCAGGCTTCCAA | |
| | CGCCGAGAGAGTGGGACTGTCTCTGTGCATCGGCTTTTCCACTTAAATC | |
| | TCTTTCACGCACAAGTATCTTTGTGCACACTCATCTATCCTAACCCCGTC | |
| | CACCCTCTTCAAGACCTGCGGCGATGGGGGAGTGGCGACGCAACAGGT | |
| | GGAGGTGACCACTGGCAGTTGTAGTCACGATCCTGCACGTAGGCGGCC | |
| | CACGGACCAGTGGTCGCTCGGCCCTGTGGGCAGCAGGGACGTTCGGCA | |
| | GCATCCTGGGCGACTGAGCAGCCCTCTCTAG (SEQ ID NO: 702) | |

Briefly, the RNAIVT354 and RNAIVT381 template RNAs for use with the RNAIVT696 gene modifying polypeptide comprised the following sequences from 5′ to 3′: 5′ UTRᵣₑₜᵣₒ, bGHpA, GFP, Kozak sequence, PGK promoter (RNAIVT354) or EF1a short promoter (RNAIVT381), 3′ UTRᵣₑₜᵣₒ. The RNAIVT610 and RNAIVT1200 template RNAs for use with the RNAIVT1123 gene modifying polypeptide comprised the following sequences from 5′ to 3′: 5′ UTRᵣₑₜᵣₒ, TKpA, GFP (RNAIVT610) or BCMA_CAR (RNAIVT1200), Kozak sequence, EF1a short promoter (RNAIVT610) or MND promoter (RNAIVT1200), 3′ UTRᵣₑₜᵣₒ.

The heterologous gene modifying system comprising the heterologous gene modifying polypeptide and the template RNA designed to produce an insertion in TRAC described above was transfected either alone or in combination with the retrotransposon gene modifying system comprising the retrotransposon gene modifying polypeptide and a template RNA encoding either GFP or a CAR in activated primary human T cells by nucleofection in RNA format. Prior to nucleofection, T cells were cultured with TexMACS supplemented with 5% human AB Serum, IL2 at 20 IU/ml, IL7 at 10 ng/ml, IL15 at 5 ng/ml in each well at 37° C., 5% CO2 for 2 days and stimulated using T Cell TransAct. For the nucleofection of the heterologous gene modifying system, 2000 ng of heterologous gene modifying polypeptide-encoding mRNA were combined with 1000 ng template RNA in RNA format. For the nucleofection of the gene modifying system, either 800 ng (RNAIVT696 gene modifying polypeptide) or 6.25 ng (RNAIVT1123 gene modifying polypeptide) of mRNA encoding the gene modifying polypeptide were combined with 1000 ng of template RNA in RNA format.

The RNA mixture (comprising either the heterologous gene modifying system or both the heterologous gene modifying system and the retrotransposon gene modifying systems) was added to 500,000 primary human HSCs in a total of 20 μL of Lonza P3 buffer and T cells were nucleofected in 16-well nucleofection cassettes using program E0-115. After nucleofection, cells incubated at room temperature for 10 minutes and were transferred to 24-well plates containing 500 μL of TexMACS supplemented with 5% human AB Serum, IL2 at 20 IU/ml, IL7 at 10 ng/ml, IL15 at 5 ng/ml in each well and cultured at 37° C., 5% CO2 for 4 days prior to flow cytometry analysis.

To determine whether insertion by the gene modifying system was successful, flow cytometry was used to determine the fraction of cells expressing GFP or BCMA CAR (as detected by staining cells with fluorescently labeled Recombinant Human BCMA Protein). To analyze cell surface markers representative of different T cells subpopulations, cells were stained with fluorescently labeled anti human CD3, CD8, CD62L, CD45RA antibodies. To determine whether editing of the TRAC locus by the heterologous gene modifying system was successful, CD3 levels were measured as a surrogate readout. Functional loss of TRAC is known to be associated with loss of surface expression of the CD3 complex.

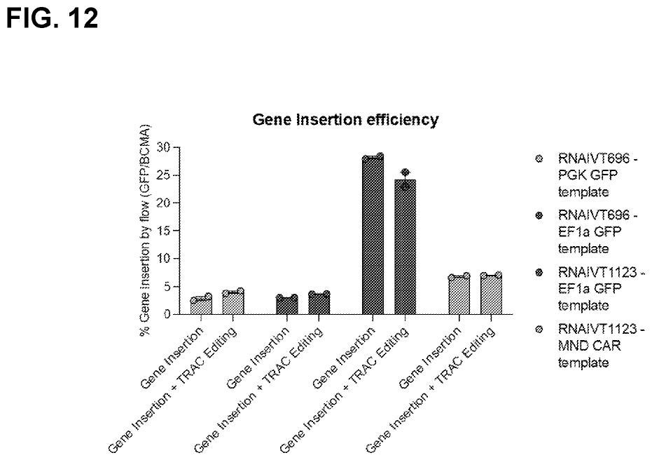

FIG. 12 shows the levels of editing by the gene modifying system in the bulk T cell population when the cells were transfected with the gene modifying system, either alone or in combination with the heterologous gene modifying system. The results show an average of 2.9% T cells expressing GFP when transfected with RNAIVT696 gene modifying polypeptide and PGK GFP template RNA, an average of 3% T cells expressing GFP when transfected with RNAIVT696 gene modifying polypeptide and EF1a GFP template RNA, an average of 28.1% T cells expressing GFP when transfected with RNAIVT1123 gene modifying polypeptide and EF1a GFP template RNA, and an average of 6.8% T cells expressing BCMA CAR when transfected with RNAIVT1123 gene modifying polypeptide and MND CAR template RNA. When these four configurations of the gene modifying system were delivered in combination with the heterologous gene modifying system, average levels of T cells expressing GFP were 4%, 3.7%, and 24.2% (respectively), and average levels of T cells expressing a CAR were 7%. The results showed that gene modifying systems using either exemplary retrotransposon gene modifying polypeptide achieved insertion of a gene into T cells, and achieved insertion of either of two exemplary genes. The results showed that editing efficiency when inserting whole genes using the gene modifying system is not affected by the co-delivery of a heterologous gene modifying system containing a heterologous gene modifying polypeptide (e.g., directing a short insertion).

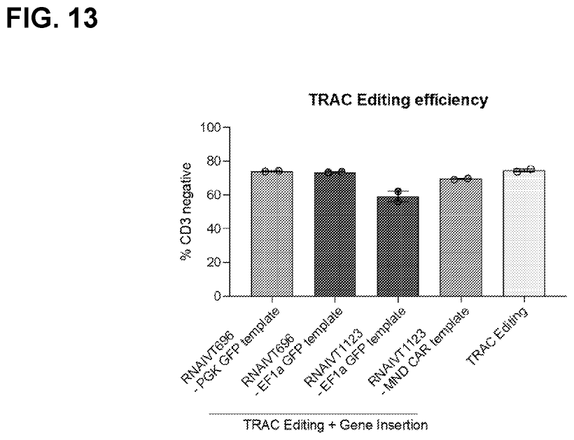

FIG. 13 shows the levels of editing by the heterologous gene modifying system in the bulk T cell population when the cells were transfected with the heterologous gene modifying system, either alone or in combination with the gene modifying system. The results showed an average of 75.3% of T cells lacking expression of CD3, used here as a surrogate marker of TRAC editing, when treated with the heterologous gene modifying system. When the heterologous gene modifying system is delivered in combination with the gene modifying system configured as indicated, levels of CD3 negative T cells were measured in the range of 59.1 to 74.1%. The results showed that heterologous gene modifying systems using an exemplary heterologous gene modifying polypeptide successfully disrupted the TRAC gene in T cells. The results show that editing efficiency when making short edits (e.g., a 5 nucleotide insertion) is not affected by the co-delivery of an exemplary gene modifying system containing a retrotransposon gene modifying polypeptide (e.g., directing a insertion of a gene).

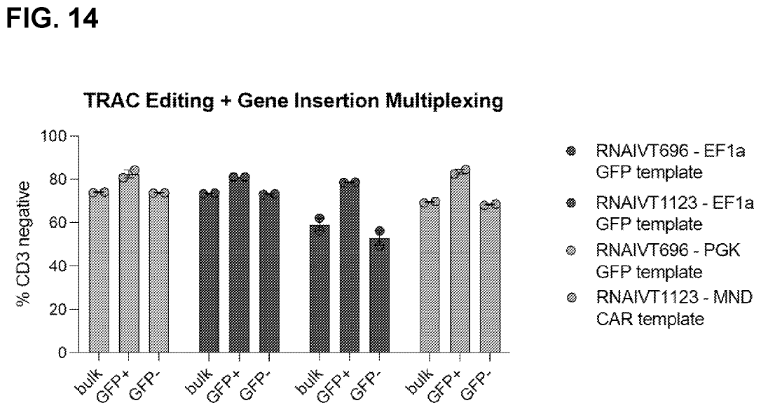

Following from the experiments of FIG. 13 demonstrating that the levels of editing in the bulk T cell population were unaffected by co-delivery of the systems, FIG. 14 shows the levels of editing by the heterologous gene modifying system (measured here as loss of expression of CD3) when the cells were co-transfected with the heterologous gene modifying system specifically breaking out TRAC editing levels in sub-populations of cells that were edited or not edited by the gene modifying system (that were GFP+ or BCMA CAR+ T cells, or GFP− or BCMA CAR−). The results demonstrated that treating T cells with both the systems results in high level of co-occurrence of the two editing events in the same cell. The similarity in editing level by the heterologous gene modifying system in cells edited or not edited by the gene modifying system further demonstrates that efficiency of editing by the heterologous gene modifying system is not affected by co-delivery of a gene modifying system.

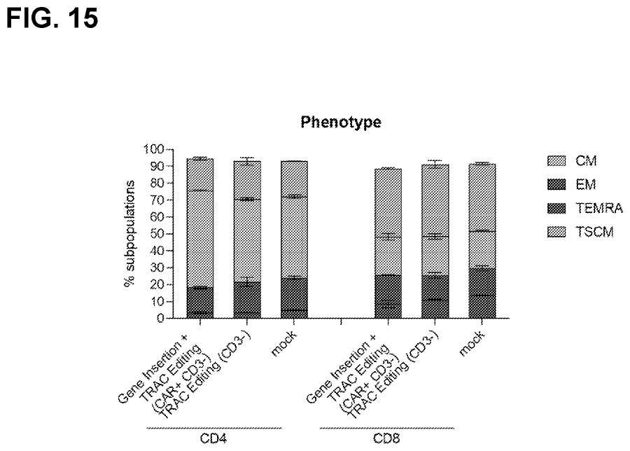

FIG. 15 shows a phenotypic characterization by flow cytometry of T cells treated with the heterologous gene modifying system, both the heterologous gene modifying system and the gene modifying system, or a mock treatment lacking both systems. Without wishing to be bound by theory, following stimulation, T cells progressively differentiate into memory and terminally differentiated effectors, acquiring effector functions and losing the ability for self-renewal and survival, and concomitant cell surface marker expression changes can be used to monitor said transitions. Stem memory T cells (TSCM) are defined as CD62L+CD45RA+, Central memory (CM) are defined as CD62L+CD45RA−, Effector memory (EM) are defined as CD62L− CD45RA, Terminal effectors (TEMRA) are defined as CD62L− CD45RA+. The distribution of the indicated T cells subpopulations within CD8 and CD4 compartments, comparing mock treated cells, CD3− cells edited with the heterologous gene modifying system and CAR+CD3− cells edited by both the heterologous gene modifying system and the gene modifying system, were graphed in FIG. 15. The results showed no skewing in the distribution of the subpopulations when comparing mock treated cells, cells edited by just the heterologous gene modifying system, and cells edited by both the systems. These results indicated that the two editing events do not impact the differentiation phenotypes of primary human T cells.

### Example 10: Evaluating Combined Gene Editing Activity of Exemplary TRAC Human Template RNAs with Gene Modifying Systems

This example describes the use of a first exemplary heterologous gene modifying system containing a heterologous gene modifying polypeptide and 2 template RNAs to produce an insertion of 5 nucleotides (TAGTG) in the first exon of the human TRAC gene and an insertion of 5 nucleotides (TAGTG) in the first exon of the Beta 2 Microglobulin (B2M) gene in primary human T cells, and a second exemplary retrotransposon gene modifying system containing a retrotransposon gene modifying polypeptide and a template RNA to insert expression cassettes for a chimeric antigen receptor (CAR) or GFP into the genome of the primary human T cells, as well as quantification of said gene modifying activities. The insertions into TRAC and B2M generated a frameshift that resulted in a non-functional gene (also referred to as a knock-out).

In this example, the template RNA of the first heterologous gene modifying system contained:
(1) a gRNA spacer;
(2) a gRNA scaffold;
(3) a heterologous object sequence; and
(4) a primer binding site (PBS) sequence.

The exemplary template RNAs generated and used in the first heterologous gene modifying system are given in Table E7. Nucleotide modifications are noted as follows: phosphorothioate linkages denoted by an asterisk, 2′-O-methyl groups denoted by an ‘m’ preceding a nucleotide.

TABLE E7
Exemplary Template RNAs and Sequences

| | | | SEQ |
|---|---|---|---|
| Name | Descriptor | Sequence | ID NO |
| RNACS5630 | TRAC4_ | mG*mA*mG*rArArUrCrArArArArUrCrGrGrUrGr | 15,004 |
| | Ins 5 | ArArUrGrUrUrUrUrArGrAmGmCmUmAmGmAmAmAm | |
| | Nick | UmAmGmCrArArGrUrUrArArArArUrArArGrGrCr | |
| | P9R18 | UrArGrUrCrCrGrUrUrArUrCrAmAmCmUmUmGmAm | |
| | | AmAmAmAmGmUmGmGmCmAmCmCmGmAmGmUmCmGmGm | |
| | | UmGmCrCrUrGrUrCrUrGrCrCrUrArUrUrUrArGr | |
| | | UrGrCrArCrCrGrA*mU*mU*mU | |
| RNACS7755 | B2m1_ | mA*mC*mU*rCrArCrGrCrUrGrGrArUrArGrCrCr | 15,005 |
| | P11R18_ | UrCrCrGrUrUrUrUrArGrAmGmCmUmAmGmAmAmAm | |
| | ins5_ | UmAmGmCrArArGrUrUrArArArArUrArArGrGrCr | |
| | nick_v1 | UrArGrUrCrCrGrUrUrArUrCrAmAmCmUmUmGmAm | |
| | | AmAmAmAmGmUmGmGmCmAmCmCmGmAmGmUmCmGmGm | |
| | | UmGmCrUrUrUrCrUrGrGrCrCrUrGrGrArUrGrAr | |
| | | UrGrGrGrCrUrArUrCrC*mA*mG*mC | |

Table E7A shows the sequences of Table E7 without chemical modifications. In some embodiments, the sequences of Table E7A may be used without chemical modifications, or with one or more chemical modifications.

TABLE E7A
Table E7 Sequences without Chemical Modifications

| | | | SEQ |
|---|---|---|---|
| RNACS | Name | Sequence | ID NO |
| RNACS5630 | TRAC4_ | GAGAAUCAAAAUCGGUGAAUGUUUUAGAGCUAGAAAUAGC | 15,006 |
| | Ins5 | AAGUUAAAAUAAGGCUAGUCCGUUAUCAACUUGAAAAAGU | |
| | Nick | GGCACCGAGUCGGUGCCUGUCUGCCUAUUUAGUGCACCGA | |
| | P9R18 | UUU | |
| RNACS7755 | B2m1_ | ACUCACGCUGGAUAGCCUCCGUUUUAGAGCUAGAAAUAGC | 15,007 |
| | P11R18_ | AAGUUAAAAUAAGGCUAGUCCGUUAUCAACUUGAAAAAGU | |
| | ins5_ | GGCACCGAGUCGGUGCUUUCUGGCCUGGAUGAUGGGCUAU | |
| | nick_v1 | CCAGC | |

In this example, exemplary heterologous gene modifying polypeptides of the first heterologous gene modifying system contained:
(1) an endonuclease and/or DNA binding domain;
(2) a peptide linker; and
(3) a reverse transcriptase (RT) domain,
   where (1), (2), and (3) are heterologous to each other. Accordingly, the first heterologous gene modifying polypeptides in this Example were examples of heterologous gene modifying polypeptides.

The exemplary gene heterologous modifying polypeptide of the first gene modifying system was RNAIVT1564, and comprised the amino acid sequence indicated in Table E8.

TABLE E8
Exemplary heterologous gene modifying polypeptide and Sequence

| Name | AA sequence | SEQ ID NO |
|---|---|---|
| RNAIVT1564 | MPAAKRVKLDGGDKKYSIGLDIGTNSVGWAVITDEYKVPSK | 15,008 |
| | KFKVLGNTDRHSIKKNLIGALLFDSGETAEATRLKRTARRRY | |
| | TRRKNRICYLQEIFSNEMAKVDDSFFHRLEESFLVEEDKKHER | |
| | HPIFGNIVDEVAYHEKYPTIYHLRKKLVDSTDKADLRLIYLAL | |
| | AHMIKFRGHFLIEGDLNPDNSDVDKLFIQLVQTYNQLFEENPI | |
| | NASGVDAKAILSARLSKSRRLENLIAQLPGEKKNGLFGNLIAL | |
| | SLGLTPNFKSNFDLAEDAKLQLSKDTYDDDLDNLLAQIGDQY | |
| | ADLFLAAKNLSDAILLSDILRVNTEITKAPLSASMIKRYDEHH | |
| | QDLTLLKALVRQQLPEKYKEIFFDQSKNGYAGYIDGGASQEE | |
| | FYKFIKPILEKMDGTEELLVKLNREDLLRKQRTFDNGSIPHQI | |
| | HLGELHAILRRQEDFYPFLKDNREKIEKILTFRIPYYVGPLARG | |
| | NSRFAWMTRKSEETITPWNFEEVVDKGASAQSFIERMTNFDK | |
| | NLPNEKVLPKHSLLYEYFTVYNELTKVKYVTEGMRKPAFLSG | |
| | EQKKAIVDLLFKTNRKVTVKQLKEDYFKKIECFDSVEISGVED | |
| | RFNASLGTYHDLLKIIKDKDFLDNEENEDILEDIVLTLTLFEDR | |
| | EMIEERLKTYAHLFDDKVMKQLKRRRYTGWGRLSRKLINGI | |
| | RDKQSGKTILDFLKSDGFANRNFMQLIHDDSLTFKEDIQKAQ | |
| | VSGQGDSLHEHIANLAGSPAIKKGILQTVKVVDELVKVMGRH | |
| | KPENIVIEMARENQTTQKGQKNSRERMKRIEEGIKELGSQILK | |
| | EHPVENTQLQNEKLYLYYLQNGRDMYVDQELDINRLSDYDV | |
| | DHIVPQSFLKDDSIDNKVLTRSDKARGKSDNVPSEEVVKKMK | |
| | NYWRQLLNAKLITQRKFDNLTKAERGGLSELDKAGFIKRQLV | |
| | ETRQITKHVAQILDSRMNTKYDENDKLIREVKVITLKSKLVSD | |
| | FRKDFQFYKVREINNYHHAHDAYLNAVVGTALIKKYPKLESE | |
| | FVYGDYKVYDVRKMIAKSEQEIGKATAKYFFYSNIMNFFKTE | |
| | ITLANGEIRKRPLIETNGETGEIVWDKGRDFATVRKVLSMPQV | |
| | NIVKKTEVQTGGFSKESILPKRNSDKLIARKKDWDPKKYGGF | |
| | DSPTVAYSVLVVAKVEKGKSKKLKSVKELLGITIMERSSFEK | |
| | NPIDFLEAKGYKEVKKDLIIKLPKYSLFELENGRKRMLASAGE | |
| | LQKGNELALPSKYVNFLYLASHYEKLKGSPEDNEQKQLFVEQ | |
| | HKHYLDEIIEQISEFSKRVILADANLDKVLSAYNKHRDKPIRE | |
| | QAENIIHLFTLTNLGAPAAFKYFDTTIDRKRYTSTKEVLDATLI | |
| | HQSITGLYETRIDLSQLGGDSGGSSGGSSGSETPGTSESATPES | |
| | SGGSSGGSSTLNIEDEYRLHETSKEPDVSLGSTWLSDFPQAWA | |
| | ETGGMGLAVRQAPLIIPLKATSTPVSIKQYPMSQEARLGIKPHI | |
| | QRLLDQGILVPCQSPWNTPLLPVKKPGTNDYRPVQDLREVNK | |
| | RVEDIHPTVPNPYNLLSGLPPSHQWYTVLDLKDAFFCLRLHPT | |
| | SQPLFAFEWRDPEMGISGQLTWTRLPQGFKNSPTLFNEALHR | |
| | DLADFRIQHPDLILLQYVDDLLLAATSELDCQQGTRALLQTL | |
| | GNLGYRASAKKAQICQKQVKYLGYLLKEGQRWLTEARKET | |
| | VMGQPTPKTPRQLREFLGKAGFCRLFIPGFAEMAAPLYPLTKP | |
| | GTLFNWGPDQQKAYQEIKQALLTAPALGLPDLTKPFELFVDE | |
| | KQGYAKGVLTQKLGPWRRPVAYLSKKLDPVAAGWPPCLRM | |
| | VAAIAVLTKDAGKLTMGQPLVILAPHAVEALVKQPPDRWLS | |
| | NARMTHYQALLLDTDRVQFGPVVALNPATLLPLPEEGLQHN | |
| | CLDILAEAHGTRPDLTDQPLPDADHTWYTDGSSLLQEGQRKA | |
| | GAAVTTETEVIWAKALPAGTSAQRAELIALTQALKMAEGKK | |
| | LNVYTDSRYAFATAHIHGEIYRRRGWLTSEGKEIKNKDEILAL | |
| | LKALFLPKRLSIIHCPGHQKGHSAEARGNRMADQAARKAAIT | |
| | ETPDTSTLLIENSSPSGGSKRTADGSEFEKRTADGSEFESPKKK | |
| | AKVE | |

In this example, exemplary gene modifying polypeptides of the second retrotransposon gene modifying system contained:
(3) an endonuclease and/or DNA binding domain; and
(4) a reverse transcriptase (RT) domain,
   where (1) and (2) are both derived from a retrotransposon (and in this Example, from the same retrotransposons). Accordingly, the second gene modifying polypeptides in this Example were examples of retrotransposon gene modifying polypeptides.

The second retrotransposon gene modifying system used in this Example comprised either of two exemplary retrotransposon gene modifying polypeptides: RNAIVT696 (SEQ ID NO: 15,002) and RNAIVT1123 (SEQ ID NO: 15,003), and amino acid sequences are given in Table E9.

TABLE E9
Exemplary gene modifying polypeptide and Sequences

| Name | | SEQ ID |
|---|---|---|
| RNAI | AA sequence | NO |
| VT696 | MDEYQRSLSRPLLTIMSINIEGLSLAKEELLAKMSEDISCDILCIQET | 15,002 |
| | HRDITMRRPKILGMQLAVERPHRQYGSAIFVRSGVAISATSLTEVN | |
| | NIEILSVELDSCTVSSLYKPPGADFYFTPPTSCHNHEAHFVVGDENS | |
| | HSCVWGYDEDDRNGEAVLTWADNSRMSLLHDSKLPPSFNSGRW | |
| | KRGYNPDLIFVKESISHQCTKRVLNPIPNTQHRPICCVAYAAVRPK | |
| | SVPFRRRYNFNKANWTKFTETLEAAISDIEPSIENYDLFVEAVKRSS | |
| | RLSIPRGCRTSYLPGLNEESLNQLQEYLRLFQENPYSDGTIAAGQK | |
| | LSTALANAKKDRWIELLENLDMSKSSRKAWQLLRRLDSDPLVNP | |
| | GHANVTPDQIAHQLIQNGKTNCSRIKMKINRVPELETHQLSSPLNL | |
| | KELREAIKRCKTGKAPGLDDLMMEQIKHLGAKAENWLLKFYNQC | |
| | LAHKQIPRAWRKTKIIAILKPGKDASNARNYRPISLLCHLYKVYER | |
| | MLLNRLGPVIEPKLIAQQAGFRPGKNCTGQILHLTEHIEEGYEKGCI | |
| | TGTVFVDLTAAYDTVQHRKMLHKVYHITRDFDFTKTVQTLLENR | |
| | SFYVEFQGQKSRWRRQKNGLPQGSVLAPTLFNIFTNDQPQPPLTKS | |
| | FIYADDLGLTTQAKDFETVEKQLTNALKDLSSYYKENHLKPNPAK | |
| | TQVCAFHLRNREANRKLKVTWEGQELEHCFHPKYLGVTLDRTLT | |
| | YRKHCMNTKHKVAARNNILRKLTGSAWGADPQVIRTSALALSFS | |
| | TAEYACPVWHKSAHAKQVDIALNETCRIITGCLKPTPVDKLYKLA | |
| | GIAPPDVRREVAANGERKKVEHCESHPLHGYHPPPTRLKSRKGFM | |
| | RTTTPLDVPPAAARVSLWAAKPGNSNWMAPQEGLPPGANQEWA | |
| | TWKSLNRLRSGVGRSKDNLARWHYLEESSTLCDCGAEQTTQHM | |
| | YACPQCPASCTEEELFKATDNAVAVARFWSKTIGGGSPKKKRKVS | |
| | GSETPGTSESATPESVSGWRLFKKIS | |
| RNAIVT1123 | MDSTAHPNQGRGLEKVSQTLPALQTPGQHTAAGGSSPLSGRNQR | 15,003 |
| | KNTKKLLLGAWNIRTLLDRENTPRPERRTALIGKELARYNIDIAAL | |
| | SETRLPEEGSLSEPTTGYTFFWKGRASNEDRIHGVGLAIKTSLLKQ | |
| | LPDLPVGISERLMKIRLPLSKDRYATIISAYAPTLTSTEETIEQFYSD | |
| | LSAVLHSVPTNDKLILLGDFNARVGQDHERWKGVLGKHGVGKM | |
| | NNNGLLLLSKCSEFELTITNTVFRMANKYKTTWMHPRSKQWHLI | |
| | DYIIVRRRDIQDVKITRAMRGAECWTDHRLVRATLQMRIAPRHPK | |
| | RAQTVRAFYNVSRLRDPSYLQTFQSCLDDKLSAKGPLTGSSTEKW | |
| | NQFRDAVKETSKAVLGPKQRNHQDWFDENNTAIEDLLSKKNKAF | |
| | MEWQNNPNSAPKKDRFKSLQATAQREIRKMQDRWWEKKAEEIQ | |
| | RFADMKNYKQFFSALKTVYGPLKPTTTPLLSSDGDTLIKDKKGISN | |
| | RWKEHFSQLLNRPSSVDQSALDQIPQNRTIEQLDVPPSIEEVQKAIK | |
| | QMSAGKAPGKDGIPTEVYKALNGKALQAFHIVLTSIWEEEDMPPE | |
| | LRDASIVALYKNKGSRAACDNYRGISLLSTAGKILARVILNRLLSS | |
| | VSEQNLPESQCGFRPDRSTIDMVFTVRQMQEKCLEQNLSLYIVFID | |
| | LTKAFDTVNRDALWVILSKLGCPAKFVKLIQLFHVDMTGEVLSGG | |
| | ETSDRFNISNGVKQGCVLAPVLFNLFFTQVLRHAVMDLDLGVYIK | |
| | YRLDGSLFDLRRLTAKTKTTERLILEALFADDCALMAHQENHLQT | |
| | IVDRFSTATKLFGLTISLSKTEVLFQPAPGRPTNQPCITIDGTQLSNV | |
| | NTFKYLGSTIANDGSLDHEINARIQKASQALGRLRCKVLQHRGVS | |
| | TATKLKVYNAVVLSSLLYGCETWTLYRKHMKQLEQFHQRSLRSI | |
| | MRIRWQDRITNQEVLDRANSTSIEVMVLKTQLRWSGHVIRMDPQ | |
| | RIPRQVFYGELSAGLRKQGRPKKRFKDQLKSNLKWAGITPKQLEL | |
| | AASDRSSWRTHINHAATTFEDERRRRLAAARERRHQATTAPPVTT | |
| | GVPCPMCHKLCASAFGLQSHMRVHRRSGSETPGTSESATPESVSG | |
| | WRLFKKIS | |

Exemplary templates RNAs comprising sequences capable of binding the exemplary retrotransposon gene modifying polypeptides included RNAIVT381 for RNAIVT696 gene modifying polypeptide and RNAIVT610 and RNAIVT1200 for RNAIVT1123 gene modifying polypeptide. Sequences are given in Table E10.

TABLE E10
Exemplary retrotransposon gene modifying RNA template and Sequences

| | | SEQ ID |
|---|---|---|
| Name | RNA sequence (5′ to 3′) | NO |
| RNAIVT381 | gggggacacggaaagagccuccccgaagauugagugaauucagucgggcguccccuggg | 15,009 |
| | caacguuucuuguaageggccgaucuuuccaccccaaaagcauuggaugaGUUUAC | |
| | GGAUCCGAAUUCUCGACGGAUCGAUCCGAACAAACGACC | |
| | CAACACCCGUGCGUUUUAUUCUGUCUUUUUAUUGCCGAU | |
| | CCCCCGGCCGCUUUACUUGUACAGCUCGUCCAUGCCGAGA | |
| | GUGAUCCCGGCGGCGGUCACGAACUCCAGCAGGACCAUG | |
| | UGAUCGCGCUUCUCGUUGGGGUCUUUGCUCAGGGCGGAC | |
| | UGGGUGCUCAGGUAGUGGUUGUCGGGCAGCAGCACGGGG | |
| | CCGUCGCCGAUGGGGGUGUUCUGCUGGUAGUGGUCGGCG | |
| | AGCUGCACGCUGCCGUCCUCGAUGUUGUGGCGGAUCUUG | |
| | AAGUUCACCUUGAUGCCGUUCUUCUGCUUGUCGGCCAUG | |
| | AUAUAGACGUUGUGGCUGUUGUAGUUGUACUCCAGCUUG | |
| | UGCCCCAGGAUGUUGCCGUCCUCCUUGAAGUCGAUGCCC | |
| | UUCAGCUCGAUGCGGUUCACCAGGGUGUCGCCCUCGAAC | |
| | UUCACCUCGGCGCGGGUCUUGUAGUUGCCGUCGUCCUUG | |
| | AAGAAGAUGGUGCGCUCCUGGACGUAGCCUUCGGGCAUG | |
| | GCGGACUUGAAGAAGUCGUGCUGCUUCAUGUGGUCGGGG | |
| | UAGCGGCUGAAGCACUGCACGCCGUAGGUCAGGGUGGUC | |
| | ACGAGGGUGGGCCAGGGCACGGGCAGCUUGCCGGUGGUG | |
| | CAGAUGAACUUCAGGGUCAGCUUGCCGUAGGUGGCAUCG | |
| | CCCUCGCCCUCGCCGGACACGCUGAACUUGUGGCCGUUUA | |
| | CGUCGCCGUCCAGCUCGACCAGGAUGGGCACCACCCCGGU | |
| RNAIVT610 | GAACAGCUCCUCGCCCUUGCUCACCAUGGUGGCUUUACC | 15,010 |
| | AACAGUACCGGAAUGCCAAGCUUGGGUCcuguguucuggcggcaa | |
| | acccguugcgaaaaagaacguucacggcgacuacugcacuuauauacgguucucccccac | |
| | ccucgggaaaaaggcggagccaguacacgacaucacuuucccaguuuaccccgcgccacc | |
| | uucucuaggcaccggaucaauugccgaccccuccccccaacuucucggggacugugggc | |
| | uauuugaaauguauuuguuguagcaaugcuuuugacacgaaauaaauaaa | |
| | ggguguauggggugcucagggagggguaguaucucugguauggagggcuugucgugc | |
| | ccuccuagggcagcucuccagccucugacccccaccugacacccagcucucacuuguggc | |
| | ucccaguagcugcuagcauguggcagcggccacaccccgggcaacggcuucgacaggcc | |
| | ggcuaaaccuugugaggguagccaucgggucaucgaccccuggugaaccagggcuuugc | |
| | ucacccagcaugugaagacugcuucggcugaacagacggaagaaaccaauaagaagguuc | |
| | aacggcugagagggcgacgcagcaaagcacuguggagugcuuagggcguguuggagcac | |
| | aaaggacaacacggccauccaaugcagcugaggaagucuccagauguaacaauuuuucg | |
| | ugccacuggacccaggcuuccaacgccgagagagugggacugucucugugcaucggcuu | |
| | uuccacuuaaaucucuuucacgcacaaguaucuuugugcacacucaucuauccuaacccc | |
| | guccacccucuucaagaccugcggcgaugggggaguggcgacgcaacagguggagguga | |
| | ccacuggcaguuguagucacgauccugcacguaggggcccacggaccaguggucgcuc | |
| | ggcccugugggcagcagggacguucggcagcauccugggcgacugagcagcccucucua | |
| | gguuuacggauccgaauucucgacggaucgauccgaacaaacgacccaacacccgugcgu | |
| | gccgagagugaucccggcggcggucacgaacuccagcaggaccaugugaucgcgcuucu | |
| | cguuggggucuuugcucagggggacugggugcucagguagugguugucgggcagca | |
| | gcacggggccgucgccgauggggguguucugcugguaguggucggcgagcugcacgcu | |
| | ucggccaugauauagacguuguggcuguuguaguuguacuccagcuugugccccagga | |
| | uguugccguccuccuugaagucgaugcccuucagcucgaugcgguucaccaggguguc | |
| | gcccucgaacuucaccucggggggucuuguaguugccgucguccuugaagaagaugg | |
| | ugcgcuccuggacguagccuucgggcauggcggacuugaagaagucgugcugcuucau | |
| | guggucgggguagcggcugaagcacugcacgccguaggucaggguggucacgagggug | |
| | ggccagggcacgggcagcuugccgguggugcagaugaacuucagggucagcuugccgu | |
| | agguggcaucgcccucgcccucgccggacacgcugaacuuguggccguuuacgucgccg | |
| | uccagcucgaccaggaugggcaccaccccggugaacagcuccucgcccuugcucaccaug | |
| | guggcuuuaccaacaguaccggaaugccaagcuuggguccuguguucuggcggcaaacc | |
| | cguugcgaaaaagaacguucacggcgacuacugcacuuauauacgguucucccccacccu | |
| | cgggaaaaaggcggagccaguacacgacaucacuuucccaguuuaccccgcgccaccuuc | |
| | ucuaggcaccggaucaauugccgaccccuccccccaacuucucggggacugugggcgau | |
| | gugcgcucugcccugaaacugcacaaagacaauagucauucucgaucaccgagagacuac | |
| | cacu | |
| RNAIVT1200 | ggguguauggggugcucagggagggguaguaucucugguauggagggcuugucgugc | 15,011 |
| | ccuccuagggcagcucuccagccucugacccccaccugacacccagcucucacuuguggc | |
| | ucccaguagcugcuagcauguggcagcggccacaccccgggcaacggcuucgacaggcc | |
| | ggcuaaaccuugugaggguagccaucgggucaucgaccccuggugaaccagggcuuugc | |
| | ucacccagcaugugaagacugcuucggcugaacagacggaagaaaccaauaagaagguuc | |
| | aacggcugagagggcgacgcagcaaagcacuguggagugcuuagggcguguuggagcac | |
| | ugccacuggacccaggcuuccaacgccgagagagugggacugucucugugcaucggcuu | |
| | uuccacuuaaaucucuuucacgcacaaguaucuuugugcacacucaucuauccuaacccc | |
| | guccacccucuucaagaccugcggcgaugggggaguggcgacgcaacagguggagguga | |
| | ccacuggcaguuguagucacgauccugcacguaggggcccacggaccaguggucgcuc | |
| | ggcccugugggcagcagggacguucggcagcauccugggcgacugagcagcccucucua | |
| | gguuuacggauccgaauucucgacggaucgauccgaacaaacgacccaacacccgugcgu | |
| | cauaugcaacgcaucauaaguguccuugguggcuguagagagacccugguacagaccgu | |
| | uccccccaucucgggaucccugccccgucucuuguccagaacaucguacucuucucgcc | |
| | ugccgagauucaauucauuguagagcugauucugacccugcugauaugcgggagcaucg | |
| | gcgcuucuagaaaauuucacccuggagcgauaggcugcgaagucgcgugguggggcaua | |
| | gggcugguaaugcuugcggggggcccggggggggggagucauguucauguaguca | |
| | cugugcaggagccugcuccucuuacuccuacaauacagagugaucacaagggagagaag | |
| | caacaccccacauguuccggccaaaggggcccaaauauaaauaucacacgcgaaguccag | |
| | gccucgcguauggacggcgccuccugcugcgggccugcaagcuucaggucugaggcuca | |
| | augguuggcuggcuauaguuggagggguguggggggccuuggagcagguguugugg | |
| | ucuugaucuccaguuugguccccugaccgaaaguccaggggacgugacuggucucugca | |
| | caauaguagacgccgacguccucagccucgacccgacuaaucuucagggugaaaucggca | |
| | ccgcuaccugagccggaaaagcgauccgggaccccgcugaaacgguuggaaaccuugua | |
| | gaucaggagcuguggacuuuggcccggcuucugcagauaccagugcagguagguguug | |
| | ccguuagaguggaccagacucugggaacucuugcaggagaugcucgccggcucaccagg | |
| | ggucacagacagagacaguggagucugggucaucacgauaucgcugccacccccuccag | |
| | agccaccuccgccggagccaccgccaccggaagacacugucaccauggugcccuggcccc | |
| | agacgucgaaguaccagucauaaucauacagggaggcgcagaaauacacggcgguaucu | |
| | ucggaccugagacuagacagcuccauguaugccguagaacuugacgugucgcgggucau | |
| | gguaacccugccggugaacuucugguuguacucagaguuuccgcucgcaaaauagaucc | |
| | aacccauccacucgagucccugcccgggagccugccgcacccaauugauauaguaaucug | |
| | uccgcgccggacugaacaagcugcaccugcgggcgagcggcaugcaagagcagggcaag | |
| | aggcagaagcagggcggucaccggcagagccaugguggcuuuaccaacaguaccggaau | |
| | gccaagcuugggucugggcucuuuuuugagcucggggagcagaagcgcgcgaacagaa | |
| | gcgagaagcgaacugauugguuaguucaaauaaggcacagggucauuucagguccuugg | |
| | ggcacccuggaaacaucugaugguucucuagaaacugcugaggggggaccgcaucugg | |
| | ggaccaucuguucuuggcccugagccggggcaggaacugcuuaccacagauauccuguu | |
| | gcuugccaaaccuacagguggggucuuucauuccccccuuuuucuggagacuaaaaaa | |
| | ugaaacugcacaaagacaauagucauucucgaucaccgagagacuaccacu | |

Briefly, the RNAIVT381 template RNAs for use with the RNAIVT696 retrotransposon gene modifying polypeptide comprised the following sequences from 5′ to 3′. 5′ UTR, bGHpA, GFP, Kozak sequence, EF1a short promoter, 3′ UTR. The RNAIVT610 and RNAIVT1200 template RNAs for use with the RNAIVT1123 gene modifying polypeptide comprised the following sequences from 5′ to 3′: 5′ UTR, TKpA, GFP (RNAIVT610) or BCMA_CAR (RNAIVT1200), Kozak sequence, EF1a short promoter (RNAIVT610) or MND promoter (RNAIVT1200), 3′ UTR.

Activated primary human T cells were nucleofected with 1) the second retrotransposon gene modifying system containing a template RNA encoding either GFP or a CAR, 2) the second retrotransposon gene modifying system containing a template RNA encoding either GFP or a CAR and the first heterologous gene modifying system containing a template RNA designed to produce an insertion in TRAC or B2M, or 3) the second retrotransposon gene modifying system containing a template RNA encoding either GFP or a CAR and the first heterologous gene modifying system containing a template RNA designed to produce an insertion in TRAC and a template RNA designed to produce an insertion in B2M. The gene modifying systems were delivered in RNA format. Prior to nucleofection, T cells were cultured with TexMACS supplemented with 5% human AB Serum, IL2 at 20 IU/ml, IL7 at 10 ng/ml, IL15 at 5 ng/ml in each well at 37° C., 5% C02 for 2 days and stimulated using T Cell TransAct. For the nucleofection of the first heterologous gene modifying system, 1000 ng of heterologous gene modifying polypeptide encoding mRNA were combined with each of the 1000 ng template RNAs in RNA format. For the nucleofection of the second retrotransposon gene modifying system, either 800 ng (RNAIVT696 retrotransposon gene modifying polypeptide) or 6.25 ng (RNAIVT1123 retrotransposon gene modifying polypeptide) of mRNA encoding the retrotransposon gene modifying polypeptide were combined with 1000 ng of template RNA in RNA format.

The RNA mixture (comprising either the first heterologous gene modifying system or both the first heterologous and second retrotransposon gene modifying systems) was added to 500,000 primary human T cells in a total of 20 μL of Lonza P3 buffer and T cells were nucleofected in 16-well nucleofection cassettes using program DS-130. After nucleofection, cells incubated at room temperature for 10 minutes and were transferred to 24-well plates containing 500 μL of TexMACS supplemented with 5% human AB Serum, IL2 at 20 IU/ml, IL7 at 10 ng/ml, IL15 at 5 ng/ml in each well and cultured at 37° C., 5% C02 for 4 days prior to flow cytometry analysis.

To determine whether insertion by the second retrotransposon gene modifying system was successful, flow cytometry was used to determine the fraction of cells expressing GFP or BCMA CAR as detected by staining cells with fluorescently labeled Recombinant Human BCMA Protein. To analyze cell surface markers representative of different T cells subpopulations, cells were stained with fluorescently labeled anti human CD3, CD8, CD62L, CD45RA antibodies. To determine whether editing of the TRAC locus by the first heterologous gene modifying system was successful, CD3 levels were measured by flow cytometry as a surrogate readout. Functional loss of TRAC is known to be associated with loss of surface expression of the CD3 complex. To determine whether editing of the B2M locus by the first heterologous gene modifying system was successful, expression of B2M was measured by flow cytometry.

To measure T cell effector functions, expression of TNFa, IL2 and IFNy cytokines was measured upon stimulation. Specifically, edited T cells were stimulated for 6 hours with PMA (50 ng/ml) and Ionomycin (1 μg/ml) in presence of 1 μl/ml of Golgi Transport Inhibitor. Cells were then stained for surface antigens, fixed and permeabilized using Cytofix/Cytoperm Kit (BD) according to the manufacturer's instruction. After fixation and permeabilization, cells were stained using antibodies recognizing human TNFa, IL2, and IFNy cytokines.

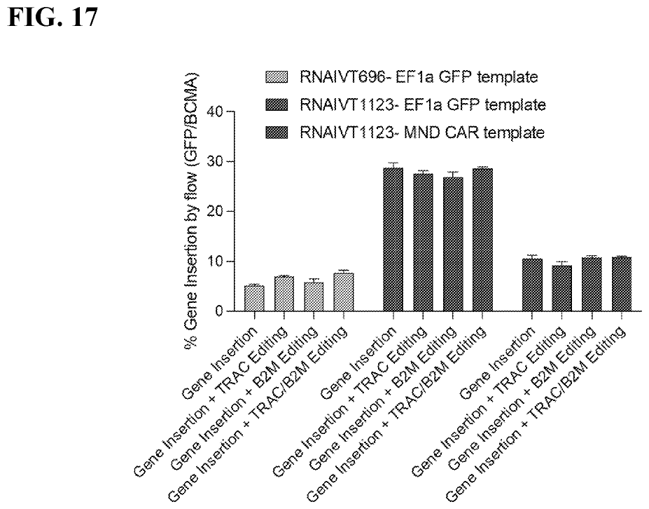

FIG. 17 shows the levels of editing by the second retrotransposon gene modifying system in the bulk T cell population when the cells were transfected with the second retrotransposon gene modifying system, either alone or in combination with the first heterologous gene modifying system. The results show an average of 5.1% T cells expressing GFP when transfected with RNAIVT696 retrotransposon gene modifying polypeptide and EF1A GFP template RNA, an average of 28.8% T cells expressing GFP when transfected with RNAIVT1123 retrotransposon gene modifying polypeptide and EF1a GFP template RNA, and an average of 10.5% T cells expressing BCMA CAR when transfected with RNAIVT1123 retrotransposon gene modifying polypeptide and MND CAR template RNA. When these 3 configurations of the second retrotransposon gene modifying system were delivered in combination with the first heterologous gene modifying system (targeting TRAC, B2M, or both loci), average levels of T cells expressing GFP or CAR were not significantly changed. For example, in the condition where both TRAC and B2M are edited, the results show an average of 7.6% T cells expressing GFP for RNAIVT696+EF1A GFP template RNA, an average of ˜28% T cells expressing GFP for RNAIVT1123+EF1a GFP template RNA, and an average of 10.9% T cells expressing BCMA CAR for RNAIVT1123+MND CAR template RNA. The results showed that gene modifying systems inserting whole genes can function in combination with gene modifying systems directing other edits (e.g., short insertions). The results further showed that editing efficiency when inserting whole genes using the second retrotransposon gene modifying system is not affected by the co-delivery of another exemplary gene modifying system containing a heterologous gene modifying polypeptide which directs one or multiple edits (e.g. short insertions).

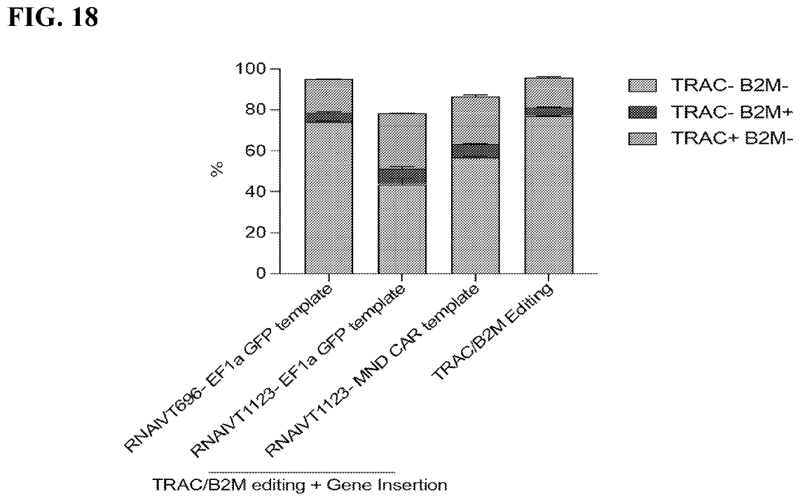

FIG. 18 shows the levels of editing by the first heterologous gene modifying system (containing a template RNA designed to produce an insertion in TRAC and a template RNA designed to produce an insertion in B2M) in the bulk T cell population when the cells were transfected with the first heterologous gene modifying system either alone or in combination with the second retrotransposon gene modifying system. The results showed an average of 95.8% of cells were edited at one or both of the TRAC or B2M loci when treated with the first heterologous gene modifying system as measured by flow cytometry. Specifically, 77% of cells showed editing in both TRAC (measured by lack of expression of CD3) and B2M, 4.3% of cells showed editing only in TRAC and 14.5% of cells showed editing only in B2M. When the first heterologous gene modifying system was delivered in combination with the second retrotransposon gene modifying system, levels of CD3 negative T cells were measured in the range of 94.9 to 78.21% and the levels of editing of both TRAC and B2M in the range of 74.2 to 46.3%. The results showed that gene modifying systems using an exemplary heterologous gene modifying polypeptide successfully disrupted both the TRAC and B2M genes in T cells. These results further show that heterologous gene modifying systems produce edits sufficient to knock out gene function when used in combination with an additional retrotransposon gene modifying systems designed to produce whole gene insertions. The results show that editing efficiency when making multiple short edits (e.g., multiple 5 nucleotide insertions) is not substantially affected by the co-delivery of another exemplary gene modifying system containing a retrotransposon gene modifying polypeptide (e.g., directing a insertion of a gene).

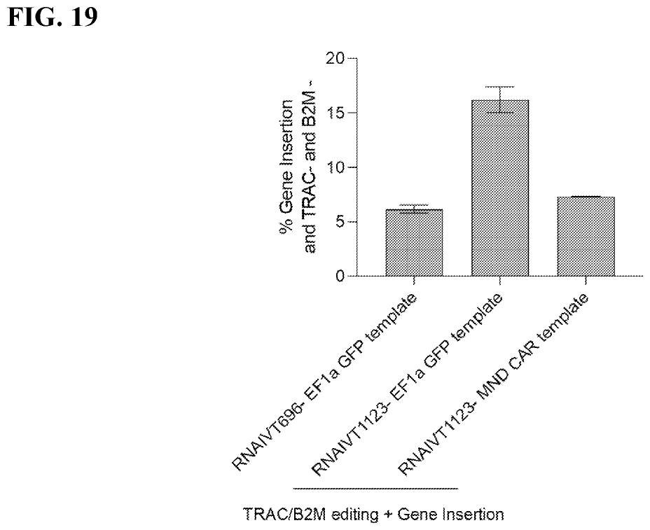

FIG. 19 shows the levels of cells edited by the first heterologous gene modifying system (measured here as combined loss of expression of CD3 and B2M by flow cytometry) and also edited by the second retrotransposon gene editing system (that were GFP+ or BCMA CAR+ T cells). The results demonstrated that treating T cells with both the first and second gene modifying systems results in substantial levels of co-occurrence of the three editing events in the same cells. For example, the results showed an average of ˜6% T cells lacking TRAC and B2M and also expressing GFP for RNAIVT696+EF1A GFP template RNA, an average of ˜16% T cells lacking TRAC and B2M and also expressing GFP for RNAIVT1123+EF1a GFP template RNA, and an average of ˜7% T cells lacking TRAC and B2M and also expressing BCMA CAR for RNAIVT1123+MND CAR template RNA.

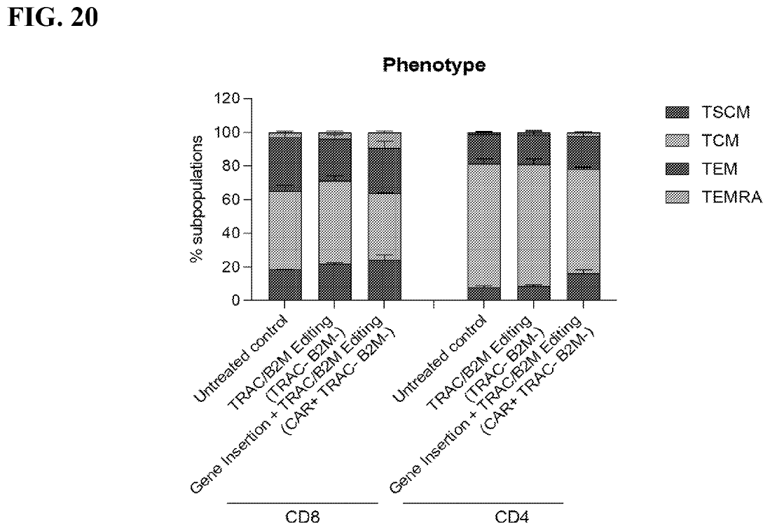

FIG. 20 shows a phenotypic characterization by flow cytometry of T cells treated with the first heterologous gene modifying system (containing a template RNA designed to produce an insertion in TRAC and a template RNA designed to produce an insertion in B2M), both the first heterologous and second retrotransposon gene modifying systems, or a mock treatment lacking both gene modifying systems. Without wishing to be bound by theory, following stimulation, T cells progressively differentiate into memory and terminally differentiated effectors, acquiring effector functions and losing the ability for self-renewal and survival, and concomitant cell surface marker expression changes can be used to monitor said transitions. Stem memory T cells (TSCM) are defined as CD62L+CD45RA+, Central memory (CM) are defined as CD62L+CD45RA−, Effector memory (TEM) are defined as CD62L− CD45RA−, Terminal effectors (TEMRA) are defined as CD62L− CD45RA+. The distribution of the indicated T cells subpopulations within CD8 and CD4 compartments, comparing mock treated cells, CD3− B2M− cells edited with the first heterologous gene modifying system and CAR+CD3− B2M− cells edited by both the first heterologous and the second retrotransposon gene modifying systems, were graphed in FIG. 20. The results showed no substantial skewing in the distribution of the TSCM, CM, TEM, and TEMRA subpopulations when comparing mock treated cells, cells edited by just the first heterologous gene modifying system comprising both TRAC and B2M edits, and cells edited by both the first heterologous and second retrotransposon gene modifying systems. These results demonstrated that co-occurrence of three editing events (e.g., two by the first heterologous gene modifying system and one by the second retrotransposon gene modifying system) does not impact the differentiation phenotypes of primary human T cells.

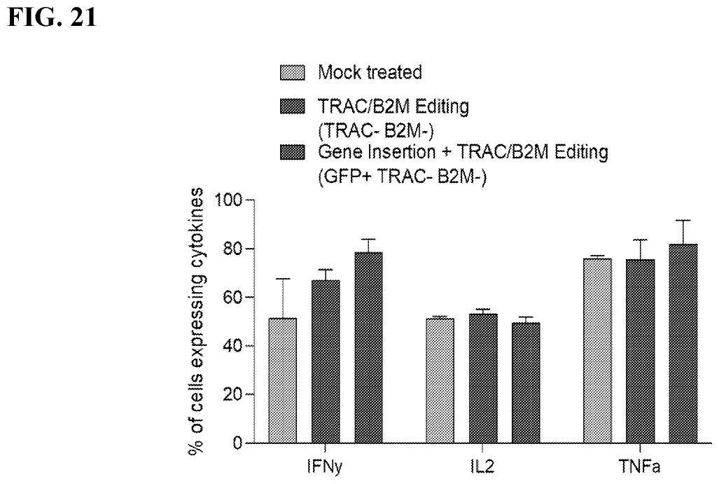

FIG. 21 shows a functional characterization by flow cytometry of T cells treated with the first heterologous gene modifying system (containing a template RNA designed to produce an insertion in TRAC and a template RNA designed to produce an insertion in B2M), both the first heterologous and second retrotransposon gene modifying systems, or a mock treatment lacking both gene modifying systems. Expression of cytokines (such as TNFa, IL2, IFNy) is a prerequisite for T cells to perform effector functions. The results showed no substantial alteration in the fraction of T cells capable of secreting the indicated cytokines when comparing mock treated cells, cells edited by just the first heterologous gene modifying system comprising both edits, and cells edited by both the first heterologous and second retrotransposon gene modifying systems. These results demonstrated that co-occurrence of three editing events (e.g., two by the first heterologous gene modifying system and one by the second retrotransposon gene modifying system) does not impact the basal effector functions of primary human T cells.

### Example 11: Evaluating Translocation Post Multiplexed Gene Editing

This example compares the use exemplary heterologous gene modifying systems containing (i) a heterologous gene modifying polypeptide comprising either (a) a wild type (WT) Cas9-RT fusion protein or (b) an exemplary gene modifying polypeptide; and (ii) and 2 template RNAs to produce an insertion of 5 nucleotides in the human TRAC gene and an insertion of 5 nucleotides in the Beta 2 Microglobulin (B2M) gene in activated T cells, as well as evaluation of said gene modifying activities for translocation events.

Table E11 shows the reagents and components used in two gene modifying systems and control (mock) groups. Sequences of the reagents listed in this table are provided in Tables E12 and E13.

TABLE E11
Experimental conditions

| Gene Modifying | Reagents | |
|---|---|---|
| system | used | Description |
| Gene Modifying | RNAIVT1573, | WTCas9-RT + TRAC sgRNA + |
| System 1 | RNACS6527, | B2M sgRNA |
| | RNACS6674 | |
| Gene Modifying | RNAIVT 338, | Exemplary Gene Modifying |
| System 2 | RNACS5630, | Polypeptide + TRAC tgRNA + |
| | RNACS7755 | B2M tgRNA |
| Mock | Water only | |

In this example, the template RNA of the heterologous gene modifying systems contained:
(1) a gRNA spacer;
(2) a gRNA scaffold;
(3) a heterologous object sequence; and
(4) a primer binding site (PBS) sequence.
   The exemplary template RNAs generated and used in the gene modifying systems and the corresponding dosages used in the experiments are given in Table E12.

TABLE E12
Template and single guide RNA: Names, sequences and doses

| Description | Name | Sequence | Dosage |
|---|---|---|---|
| TRAC4_P9R18_ | RNACS5630 | GAGAAUCAAAAUCGGUGAAUGUUUUAGAGCU | 1.5 μM |
| ins5-nick | | AGAAAUAGCAAGUUAAAAUAAGGCUAGUCCG | |
| | | UUAUCAACUUGAAAAAGUGGCACCGAGUCGGU | |
| | | GCCUGUCUGCCUAUUUAGUGCACCGAUUU | |
| | | (SEQ ID NO: 15006) | |
| B2m1_P1IR18_ | RNACS7755 | ACUCACGCUGGAUAGCCUCCGUUUUAGAGCUA | 1.5 μM |
| ins5_nick_ | | GAAAUAGCAAGUUAAAAUAAGGCUAGUCCGU | |
| v1 | | UAUCAACUUGAAAAAGUGGCACCGAGUCGGUG | |
| | | CUUUCUGGCCUGGAUGAUGGGCUAUCCAGC | |
| | | (SEQ ID NO: 15007) | |
| TRAC4_sgRNA | RNACS6527 | GAGAAUCAAAAUCGGUGAAUGUUUUAGAGCU | 1.5 μM |
| | | AGAAAUAGCAAGUUAAAAUAAGGCUAGUCCG | |
| | | UUAUCAACUUGAAAAAGUGGCACCGAGUCGGU | |
| | | GCUUUU (SEQ ID NO: 15456) | |
| B2M1_sgRNA | RNACS6674 | ACUCACGCUGGAUAGCCUCCGUUUUAGAGCUA | 1.5 μM |
| | | GAAAUAGCAAGUUAAAAUAAGGCUAGUCCGU | |
| | | UAUCAACUUGAAAAAGUGGCACCGAGUCGGUG | |
| | | CUUUU (SEQ ID NO: 15457) | |

In this example, the exemplary heterologous gene modifying polypeptide of the heterologous gene modifying system contained:
(1) an endonuclease and/or DNA binding domain;
(2) a peptide linker; and
(3) a reverse transcriptase (RT) domain,
   where (1), (2), and (3) are heterologous to each other. Accordingly, the heterologous gene modifying polypeptide in this Example was an example of a heterologous gene modifying polypeptide. A fusion polypeptide comprising a wildtype Cas9 domain, a linker, and an exemplary reverse transcriptase (RT) domain was used as a double strand break-inducing comparator to the exemplary heterologous gene modifying polypeptide.

The exemplary gene heterologous modifying polypeptide and fusion polypeptide of the gene modifying systems comprised the amino acid sequence indicated in Table E13.

TABLE E13
Exemplary Fusion and Gene Modifying Polypeptide
Information: Names, sequence and doses

| Polypeptide | | |
|---|---|---|
| Name/ | | |
| Description | Sequence | Dosage |
| RNAIVT1573/ | MPAAKRVKLDGGDKKYSIGLDIGTNSVGWAVIT | 2 μg |
| WT Cas9-RT | DEYKVPSKKFKVLGNTDRHSIKKNLIGALLFDSGE | |
| | TAEATRLKRTARRRYTRRKNRICYLQEIFSNEMA | |
| | KVDDSFFHRLEESFLVEEDKKHERHPIFGNIVDEV | |
| | AYHEKYPTIYHLRKKLVDSTDKADLRLIYLALAH | |
| | MIKFRGHFLIEGDLNPDNSDVDKLFIQLVQTYNQL | |
| | FEENPINASGVDAKAILSARLSKSRRLENLIAQLPG | |
| | EKKNGLFGNLIALSLGLTPNFKSNFDLAEDAKLQL | |
| | SKDTYDDDLDNLLAQIGDQYADLFLAAKNLSDAI | |
| | LLSDILRVNTEITKAPLSASMIKRYDEHHQDLTLL | |
| | KALVRQQLPEKYKEIFFDQSKNGYAGYIDGGASQ | |
| | EEFYKFIKPILEKMDGTEELLVKLNREDLLRKORT | |
| | FDNGSIPHQIHLGELHAILRRQEDFYPFLKDNREKI | |
| | EKILTFRIPYYVGPLARGNSRFAWMTRKSEETITP | |
| | WNFEEVVDKGASAQSFIERMTNFDKNLPNEKVLP | |
| | KHSLLYEYFTVYNELTKVKYVTEGMRKPAFLSGE | |
| | QKKAIVDLLFKTNRKVTVKQLKEDYFKKIECFDS | |
| | VEISGVEDRFNASLGTYHDLLKIIKDKDFLDNEEN | |
| | EDILEDIVLTLTLFEDREMIEERLKTYAHLFDDKV | |
| | MKQLKRRRYTGWGRLSRKLINGIRDKQSGKTILD | |
| | FLKSDGFANRNFMQLIHDDSLTFKEDIQKAQVSG | |
| | QGDSLHEHIANLAGSPAIKKGILQTVKVVDELVK | |
| | VMGRHKPENIVIEMARENQTTQKGQKNSRERMK | |
| | RIEEGIKELGSQILKEHPVENTQLQNEKLYLYYLQ | |
| | NGRDMYVDQELDINRLSDYDVDHIVPQSFLKDDS | |
| | IDNKVLTRSDKNRGKSDNVPSEEVVKKMKNYWR | |
| | QLLNAKLITQRKFDNLTKAERGGLSELDKAGFIKR | |
| | QLVETRQITKHVAQILDSRMNTKYDENDKLIREV | |
| | KVITLKSKLVSDFRKDFQFYKVREINNYHHAHDA | |
| | YLNAVVGTALIKKYPKLESEFVYGDYKVYDVRK | |
| | MIAKSEQEIGKATAKYFFYSNIMNFFKTEITLANG | |
| | EIRKRPLIETNGETGEIVWDKGRDFATVRKVLSMP | |
| | QVNIVKKTEVQTGGFSKESILPKRNSDKLIARKKD | |
| | WDPKKYGGFDSPTVAYSVLVVAKVEKGKSKKLK | |
| | SVKELLGITIMERSSFEKNPIDFLEAKGYKEVKKD | |
| | LIIKLPKYSLFELENGRKRMLASAGELQKGNELAL | |
| | PSKYVNFLYLASHYEKLKGSPEDNEQKQLFVEQH | |
| | KHYLDEIIEQISEFSKRVILADANLDKVLSAYNKH | |
| | RDKPIREQAENIIHLFTLTNLGAPAAFKYFDTTIDR | |
| | KRYTSTKEVLDATLIHQSITGLYETRIDLSQLGGD | |
| | GGAEAAAKEAAAKEAAAKEAAAKALEAEAAAK | |
| | EAAAKEAAAKEAAAKAGGTAPLEEEYRLFLEAPI | |
| | QNVTLLEQWKREIPKVWAEINPPGLASTQAPIHV | |
| | QLLSTALPVRVRQYPITLEAKRSLRETIRKFRAAGI | |
| | LRPVHSPWNTPLLPVRKSGTSEYRMVQDLREVNK | |
| | RVETIHPTVPNPYTLLSLLPPDRIWYSVLDLKDAFF | |
| | CIPLAPESQLIFAFEWADAEEGESGQLTWTRLPQG | |
| | FKNSPTLFNEALNRDLQGFRLDHPSVSLLQYVDD | |
| | LLIAADTQAACLSATRDLLMTLAELGYRVSGKKA | |
| | QLCQEEVTYLGFKIHKGSRSLSNSRTQAILQIPVPK | |
| | TKRQVREFLGKIGYCRLFIPGFAELAQPLYAATRP | |
| | GNDPLVWGEKEEEAFQSLKLALTQPPALALPSLD | |
| | KPFQLFVEETSGAAKGVLTQALGPWKRPVAYLSK | |
| | RLDPVAAGWPRCLRAIAAAALLTREASKLTFGQD | |
| | IEITSSHNLESLLRSPPDKWLTNARITQYQVLLLDP | |
| | PRVRFKQTAALNPATLLPETDDTLPIHHCLDTLDS | |
| | LTSTRPDLTDQPLAQAEATLFTDGSSYIRDGKRYA | |
| | GAAVVTLDSVIWAEPLPIGTSAQKAELIALTKALE | |
| | WSKDKSVNIYTDSRYAFATLHVHGMIYRERGWL | |
| | TAGGKAIKNAPEILALLTAVWLPKRVAVMHCKG | |
| | HQKDDAPTSTGNRRADEVAREVAIRPLSTQATISA | |
| | GKRTADGSEFEKRTADGSEFESPKKKAKVE (SEQ | |
| | ID NO: 15458) | |
| RNAIVT 338/ | MPAAKRVKLDGGDKKYSIGLDIGTNSVGWAVIT | 2 μg |
| Exemplary | DEYKVPSKKFKVLGNTDRHSIKKNLIGALLFDSGE | |
| gene | TAEATRLKRTARRRYTRRKNRICYLQEIFSNEMA | |
| modifying | KVDDSFFHRLEESFLVEEDKKHERHPIFGNIVDEV | |
| polypeptide | AYHEKYPTIYHLRKKLVDSTDKADLRLIYLALAH | |
| | MIKFRGHFLIEGDLNPDNSDVDKLFIQLVQTYNQL | |
| | FEENPINASGVDAKAILSARLSKSRRLENLIAQLPG | |
| | EKKNGLFGNLIALSLGLTPNFKSNFDLAEDAKLQL | |
| | SKDTYDDDLDNLLAQIGDQYADLFLAAKNLSDAI | |
| | LLSDILRVNTEITKAPLSASMIKRYDEHHQDLTLL | |
| | KALVRQQLPEKYKEIFFDQSKNGYAGYIDGGASQ | |
| | EEFYKFIKPILEKMDGTEELLVKLNREDLLRKQRT | |
| | FDNGSIPHQIHLGELHAILRRQEDFYPFLKDNREKI | |
| | EKILTFRIPYYVGPLARGNSRFAWMTRKSEETITP | |
| | WNFEEVVDKGASAQSFIERMTNFDKNLPNEKVLP | |
| | KHSLLYEYFTVYNELTKVKYVTEGMRKPAFLSGE | |
| | QKKAIVDLLFKTNRKVTVKQLKEDYFKKIECFDS | |
| | VEISGVEDRFNASLGTYHDLLKIIKDKDFLDNEEN | |
| | EDILEDIVLTLTLFEDREMIEERLKTYAHLFDDKV | |
| | MKQLKRRRYTGWGRLSRKLINGIRDKQSGKTILD | |
| | FLKSDGFANRNFMQLIHDDSLTFKEDIQKAQVSG | |
| | QGDSLHEHIANLAGSPAIKKGILQTVKVVDELVK | |
| | VMGRHKPENIVIEMARENQTTQKGQKNSRERMK | |
| | RIEEGIKELGSQILKEHPVENTQLQNEKLYLYYLQ | |
| | NGRDMYVDQELDINRLSDYDVDHIVPQSFLKDDS | |
| | IDNKVLTRSDKARGKSDNVPSEEVVKKMKNYWR | |
| | QLLNAKLITQRKFDNLTKAERGGLSELDKAGFIKR | |
| | QLVETRQITKHVAQILDSRMNTKYDENDKLIREV | |
| | KVITLKSKLVSDFRKDFQFYKVREINNYHHAHDA | |
| | YLNAVVGTALIKKYPKLESEFVYGDYKVYDVRK | |
| | MIAKSEQEIGKATAKYFFYSNIMNFFKTEITLANG | |
| | EIRKRPLIETNGETGEIVWDKGRDFATVRKVLSMP | |
| | QVNIVKKTEVQTGGFSKESILPKRNSDKLIARKKD | |
| | WDPKKYGGFDSPTVAYSVLVVAKVEKGKSKKLK | |
| | SVKELLGITIMERSSFEKNPIDFLEAKGYKEVKKD | |
| | LIIKLPKYSLFELENGRKRMLASAGELQKGNELAL | |
| | PSKYVNFLYLASHYEKLKGSPEDNEQKQLFVEQH | |
| | KHYLDEIIEQISEFSKRVILADANLDKVLSAYNKH | |
| | RDKPIREQAENIIHLFTLTNLGAPAAFKYFDTTIDR | |
| | KRYTSTKEVLDATLIHQSITGLYETRIDLSQLGGD | |
| | GGAEAAAKEAAAKEAAAKEAAAKALEAEAAAK | |
| | EAAAKEAAAKEAAAKAGGTAPLEEEYRLFLEAPI | |
| | QNVTLLEQWKREIPKVWAEINPPGLASTQAPIHV | |
| | QLLSTALPVRVRQYPITLEAKRSLRETIRKFRAAGI | |
| | LRPVHSPWNTPLLPVRKSGTSEYRMVQDLREVNK | |
| | RVETIHPTVPNPYTLLSLLPPDRIWYSVLDLKDAFF | |
| | CIPLAPESQLIFAFEWADAEEGESGQLTWTRLPQG | |
| | FKNSPTLFNEALNRDLQGFRLDHPSVSLLQYVDD | |
| | LLIAADTQAACLSATRDLLMTLAELGYRVSGKKA | |
| | QLCQEEVTYLGFKIHKGSRSLSNSRTQAILQIPVPK | |
| | TKRQVREFLGKIGYCRLFIPGFAELAQPLYAATRP | |
| | GNDPLVWGEKEEEAFQSLKLALTQPPALALPSLD | |
| | KPFQLFVEETSGAAKGVLTQALGPWKRPVAYLSK | |
| | RLDPVAAGWPRCLRAIAAAALLTREASKLTFGQD | |
| | IEITSSHNLESLLRSPPDKWLTNARITQYQVLLLDP | |
| | PRVRFKQTAALNPATLLPETDDTLPIHHCLDTLDS | |
| | LTSTRPDLTDQPLAQAEATLFTDGSSYIRDGKRYA | |
| | GAAVVTLDSVIWAEPLPIGTSAQKAELIALTKALE | |
| | WSKDKSVNIYTDSRYAFATLHVHGMIYRERGWL | |
| | TAGGKAIKNAPEILALLTAVWLPKRVAVMHCKG | |
| | HQKDDAPTSTGNRRADEVAREVAIRPLSTQATISA | |
| | GKRTADGSEFEKRTADGSEFESPKKKAKVE (SEQ | |
| | ID NO: 15459) | |

### T-Cell Culturing, Activation and Nucleofection:

Activated T cells were used for this experiment. Prior to nucleofection, T cells were cultured with TexMACS supplemented with 5% human AB Serum, IL2 at 20 IU/ml, IL7 at 10 ng/ml, IL15 at 5 ng/ml in each well at 37° C., 5% C02 for 2 days and stimulated using T Cell TransAct. For the nucleofection of the first heterologous gene modifying system, 2000 ng of the first heterologous gene modifying polypeptide encoding mRNA was combined with TRAC and B2M single-guide RNAs (each sgRNA at a final conc. 1.5 μM) in RNA format. For the nucleofection of the second heterologous gene modifying system, 2000 ng of the second heterologous gene modifying polypeptide encoding mRNA was combined with TRAC and B2M template guide RNAs (each tgRNA at a final conc. 1.5 μM) in RNA format.

The RNA mixture (comprising the first or the second heterologous gene modifying system) was added to 250,000 activated T cells/condition in a total of 20 μL of Lonza P3 buffer. The T cells were then nucleofected in 96-well nucleofection cassettes using the program called Human T cell, stimulated (Program code: EO 115). After nucleofection, the cells were incubated at room temperature for 10 minutes and then transferred to 96-well plates containing 180 μL of TexMACS supplemented with 5% human AB Serum, IL2 at 20 IU/ml, IL7 at 10 ng/ml in each well and cultured at 37° C., 5% C02 for either 3 days prior to genomic extraction, or, for 4 days prior to flow cytometry analysis.

### Flow Cytometry:

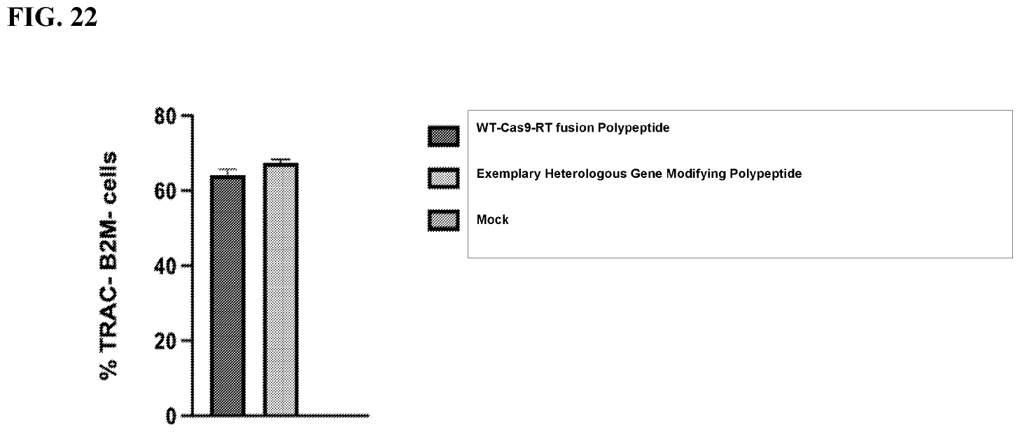

To determine whether editing of the TRAC and B2M loci by the first and second heterologous gene modifying systems was successful, CD3 and B2M levels were measured. Functional loss of TRAC is known to be associated with loss of surface expression of the CD3 complex. Similarly, functional loss of B2M is associated with the loss of surface expression of beta2-microglobulin and can be detected with B2M antibody staining. As shown in FIG. 22, editing at the TRAC and B2M loci by the first and second heterologous gene modifying systems was successful with similar editing efficiencies using gene modifying systems comprising either a WT Cas9-RT fusion polypeptide or an exemplary gene modifying polypeptide, and editing was not observed in mock treated cells.

Translocation Measurements Using Digital PCR (dPCR):

To examine translocations by dPCR, cells were collected on the third day post nucleofection. Genomic DNA was extracted using a DNAdvance kit from Beckman Coulter by lysing the cells overnight in the Lysis LBH buffer with Proteinase K, and 1M DTT. The lysed cells were extracted as per manufacturer instructions using the Biomek i5 liquid handler. Post extraction, the concentrations of extracted genomic DNA were measured using the Quant-it 1×dsDNA assay kit, High Sensitivity from Thermofisher.

Translocation events were quantitated using the QIAcuity Eight digital PCR system from Qiagen. The reaction mix was prepared with the QIAcuity Probe PCR Kit according to manufacturer instructions and loaded onto 96-well 8.5 k nanochamber plates. Reaction partitioning was performed by the instrument's default protocol. Thermocycling and imaging were performed as described below. CNV (translocation occurrence) was calculated as such: [target amplicon]/[reference gene amplicon]*ploidy. Ploidy was assumed to be 2.

| Temperature | Time | | Cycles |
|---|---|---|---|
| 95° C. hot start | 2 | min | 1 |
| 94° C. denature | 30 | sec | < |
| 58° C. anneal/ext | 1 | min | 40X> |

| | | F primer | R primer |
|---|---|---|---|
| Assay | Probe (400 nM) | (800 nM) | (800 nM) |
| Balanced | /56- | GAACTCTCTTA | CCTTTCTTGCA |
| 1 | FAM/TGGTCTTAG/ZEN/GAACTTTG | AGACTACCTTT | GTTGAGTTGTG |
| | CTGCCACA/3IABKFQ/ | CTCC (SEQ ID | (SEQ ID NO: |
| | (SEQ ID NO: 15460) | NO: 15461) | 15462) |
| Balanced | /56- | GAATCTGAAC | ATCTTAAGCTG |
| 2 | FAM/ACGACCCAT/ZEN/ACACCCA | TCTCTTCAACC | CTGGGTATTGT |
| | AAGGATTGAG/3IABKFQ/ | TTTG (SEQ ID | (SEQ ID NO: |
| | (SEQ ID NO: 15463) | NO: 15464) | 15465) |
| Dicentric | /56- | GAGGTGTCCG | AGAATCTGAAC |
| | FAM/TGGTCTTAG/ZEN/GAACTTTG | TGTTCCTAAA | TCTCTTCAACC |
| | CTGCCACA/3IABKFQ/ | (SEQ ID NO: | T (SEQ ID NO: |
| | (SEQ ID NO: 15460) | 15466) | 15467) |
| Acentric | /56- | CCTTTCTTGCA | GACCTCAATCC |
| | FAM/ACTGTAAAC/ZEN/CTACACA | GTTGAGTTGT | TTTGGGTGTAT |
| | GGTGAGTTGGC/3IABKFQ/ | G (SEQ ID NO: | (SEQ ID NO: |
| | (SEQ ID NO: 15468) | 15462) | 15469) |
| RPP30 | /5HEX/TCCCTAGGT/ZEN/GGCCTG | TCCAGGAGGG | ATGGTCCGTCT |
| | AGCAG/3IABKFQ/ | AGAATTGATG | CAGGAAA (SEQ |
| | (SEQ ID NO: 15470) or /56- | (SEQ ID NO: | ID NO: 15473) |
| | ROXN/AGAGAACTGAGTGGAGTCA | 15472) | |
| | AGGCTGA/3IAbRQSp/ | | |
| | (SEQ ID NO: 15471) | | |

| | | Exposure | |
|---|---|---|---|
| Dye | Color | time (ms) | Gain |
| FAM | Green | 600 | 6 |
| HEX | Yellow | 500 | 6 |
| ROX | Red | 300 | 4 |

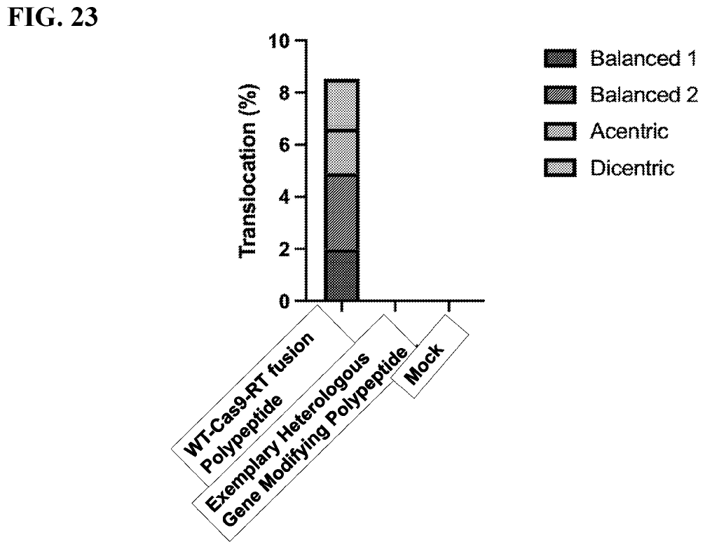

Total translocation % was calculated as a sum of all the 4 different translocation measurements (Balanced 1, Balanced 2, Acentric and Dicentric). FIG. 23 shows a graph of translocation % for cells transfected with gene modifying systems comprising WT Cas9-RT fusion polypeptide or an exemplary gene modifying polypeptide, or cells given a mock treatment. The results show that multiplexed editing with WTCas9-RT fusion polypeptide resulted in close to 8.53% translocation (FIG. 23) while editing with the exemplary heterologous gene modifying system showed background levels of translocation.

Overall, these results demonstrate that multiplexed knock-out of TRAC and B2M using an exemplary heterologous gene modifying peptide does not produce translocations in T cells, whereas a WT-Cas9-RT fusion polypeptide, which induces double strand using similarly targeted sgRNAs does produce translocations.

### Example 12: Retrotransposon-Based Gene Modifying Systems Drive the Integration and Expression of a Template in Primary Cells when Delivered as All-RNA Compositions

This example demonstrates that editing of a genome using a gene modifying system described herein drives the integration and expression of a template in primary cells when delivered as all-RNA compositions.

The gene modifying systems used in this Example comprise a nucleic acid encoding an RTE-1_MD, Vingi-1_Acar, CR1-1_PH, RTE-3_BF, or RTE-25_LMi driver as described herein.

Briefly, the template RNA encoding GFP for use with the RTE-1_MD, Vingi-1_Acar, CR1-1_PH, RTE-3_BF, and RTE-25_LMi gene modifying systems comprises the following sequences from 5′ to 3′. 5′UTR, GFP, EF1alpha, 3′UTR and was co-delivered with the driver RNA. Sequences are given in Table E14. Template RNA alone was also used as a negative control.

The gene modifying systems and any control constructs were introduced as RNAs into activated T cells, primary human bronchial epithelial (hBE) cells, or iPSC using Neon transfection system or Amaxa 4D-Nucleofector system.

Integration and expression was assessed by measuring GFP levels at 3 days post-transfection.

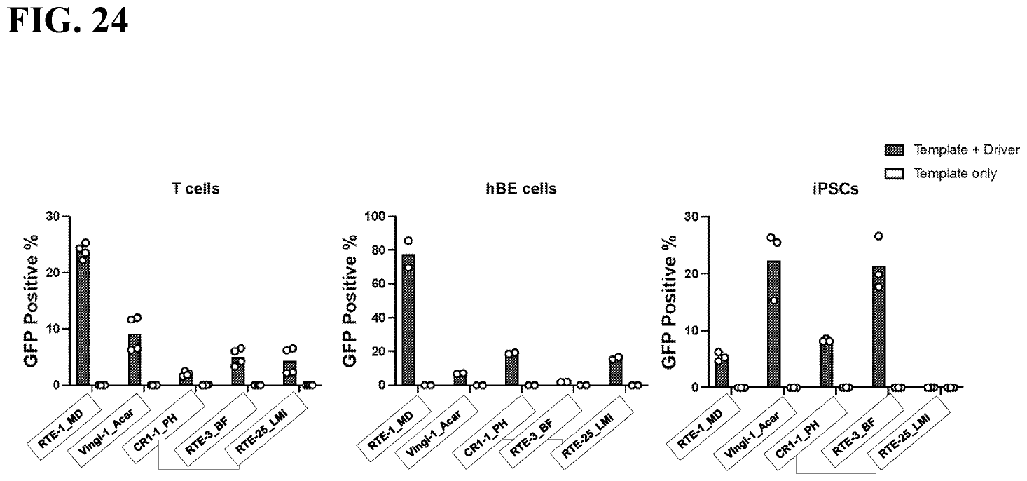

As shown in FIG. 24, RTE-1_MD, Vingi-1_Acar, CR1-1_PH, RTE-3_BF, and RTE-25_LMi gene modifying systems drive integration and expression of a template in T cells, hBE cells, iPSCs when delivered as all-RNA compositions. Successful integration of the transgene into all three cell types using several gene modifying systems is shown in FIG. 24. The use of certain drivers resulted in higher levels of expression and integration in particular cell types. For example, use of the RTE-1_MD driver resulted in the highest GFP expression levels in T and hBE cells, while use of Vingi-1_Acar and RTE-3 BF drivers resulted in the highest GFP expression levels in iPSCs

TABLE E14
Nucleic Acid and Amino Acid Sequences of Gene Modifying Polypeptides

| | | SEQ | | SEQ |
|---|---|---|---|---|
| Name | Nucleic Acid Sequence | ID NO | Amino Acid Sequence | ID NO |
| RTE- | AGGAAAUAAGAGAGAAAAGAAGAGUAAGAAGAAAUAUAAGAGCCACCAUGCCA | 15,012 | MPKKKRKVGGSDSTAHPNQGRGLEKVSQTL | 15,013 |
| 1_MD | AAGAAGAAAAGAAAAGUAGGAGGUUCCGACUCCACAGCUCAUCCCAACCAGGG | | PALQTPGQHTAAGGSSPLSGRNQRKNTKKLL | |
| RNAIVT | ACGUGGCCUGGAGAAGGUGUCCCAGACUCUGCCUGCGCUGCAGACCCCAGGGC | | LGAWNIRTLLDRENTPRPERRTALIGKELARY | |
| 1901 | AGCACACUGCCGCUGGGGGAUCCUCUCCCCUGUCCGGCAGGAAUCAGCGGAAG | | NIDIAALSETRLPEEGSLSEPTTGYTFFWKGRA | |
| | AACACCAAAAAGCUGUUGCUGGGCGCCUGGAACAUCCGCACCCUGCUCGACCGC | | SNEDRIHGVGLAIKTSLLKQLPDLPVGISERL | |
| | GAAAAUACUCCCCGUCCUGAAAGACGCACGGCACUGAUUGGGAAAGAGCUCGC | | MKIRLPLSKDRYATIISAYAPTLTSTEETIEQF | |
| | CAGAUAUAACAUCGACAUCGCUGCCCUGUCCGAAACCCGCCUGCCAGAGGAAG | | YSDLSAVLHSVPTNDKLILLGDFNARVGQDH | |
| | GAUCUCUGAGCGAGCCUACCACUGGCUACACGUUCUUUUGGAAGGGCAGAGCA | | ERWKGVLGKHGVGKMNNNGLLLLSKCSEFE | |
| | AGCAACGAGGACCGUAUCCAUGGUGUAGGCCUGGCUAUCAAGACCUCUCUCCU | | LTITNTVFRMANKYKTTWMHPRSKQWHLID | |
| | GAAACAACUCCCAGAUCUGCCAGUCGGCAUCUCAGAGAGACUGAUGAAGAUCA | | YIIVRRRDIQDVKITRAMRGAECWTDHRLVR | |
| | GGCUUCCUCUCUCAAAAGACCGCUACGCAACCAUCAUUUCCGCAUACGCGCCGA | | ATLQMRIAPRHPKRAQTVRAFYNVSRLRDPS | |
| | CCCUGACUAGCACCGAGGAAACCAUUGAGCAGUUCUACUCUGAUCUUUCAGCU | | YLQTFQSCLDDKLSAKGPLTGSSTEKWNQFR | |
| | GUUUUGCACUCCGUGCCUACCAACGACAAGCUGAUCCUCCUGGGCGACUUCAAC | | DAVKETSKAVLGPKQRNHQDWFDENNTAIE | |
| | GCUCGCGUGGGUCAGGACCAUGAGCGCUGGAAAGGCGUGCUGGGUAAGCAUGG | | DLLSKKNKAFMEWQNNPNSAPKKDRFKSLQ | |
| | CGUGGGUAAGAUGAACAAUAACGGCCUCCUGCUCCUGUCUAAGUGUAGCGAAU | | ATAQREIRKMQDRWWEKKAEEIQRFADMKN | |
| | UCGAGCUGACUAUUACCAACACUGUUUUUCGUAUGGCUAACAAGUACAAAACU | | YKQFFSALKTVYGPLKPTTTPLLSSDGDTLIK | |
| | ACAUGGAUGCACCCCAGGAGCAAGCAGUGGCACCUGAUUGACUACAUUAUCGU | | DKKGISNRWKEHFSQLLNRPSSVDQSALDQIP | |
| | GCGUAGGCGGGAUAUCCAGGACGUGAAGAUUACCCGUGCAAUGCGCGGCGCUG | | QNRTIEQLDVPPSIEEVQKAIKQMSAGKAPGK | |
| | AGUGCUGGACAGACCAUCGCCUGGUGAGAGCUACUCUCCAGAUGAGGAUCGCA | | DGIPTEVYKALNGKALQAFHIVLTSIWEEED | |
| | CCGCGUCACCCUAAGCGCGCCCAGACCGUGCGGGCCUUUUACAACGUAUCACGG | | MPPELRDASIVALYKNKGSRAACDNYRGISL | |
| | CUUCGGGAUCCUUCCUACCUUCAGACAUUUCAAUCUUGCCUCGAUGACAAGCU | | LSTAGKILARVILNRLLSSVSEQNLPESQCGFR | |
| | CUCUGCUAAGGGUCCCCUCACCGGAUCUUCAACAGAGAAGUGGAACCAAUUCC | | PDRSTIDMVFTVRQMQEKCLEQNLSLYIVFID | |
| | GCGAUGCGGUGAAGGAAACCAGCAAAGCAGUCCUGGGACCGAAGCAGCGCAAC | | LTKAFDTVNRDALWVILSKLGCPAKFVKLIQ | |
| | CACCAAGACUGGUUUGACGAAAAUAACACUGCCAUCGAAGACCUCCUGUCUAA | | LFHVDMTGEVLSGGETSDRFNISNGVKQGCV | |
| | AAAGAACAAAGCAUUUAUGGAGUGGCAGAAUAACCCAAACAGUGCACCCAAGA | | LAPVLFNLFFTQVLRHAVMDLDLGVYIKYRL | |
| | AAGACCGUUUCAAAAGCCUGCAGGCCACCGCGCAGCGCGAGAUCCGGAAGAUG | | DGSLFDLRRLTAKTKTTERLILEALFADDCAL | |
| | CAGGACCGCUGGUGGGAAAAAAAGGCCGAAGAGAUCCAGAGGUUCGCGGAUAU | | MAHQENHLQTIVDRFSTATKLFGLTISLSKTE | |
| | GAAAAACUACAAGCAGUUCUUUAGCGCACUGAAGACCGUGUACGGACCUCUCA | | VLFQPAPGRPTNQPCITIDGTQLSNVNTFKYL | |
| | AACCCACCACGACUCCCCUGCUCUCUUCCGAUGGUGACACUCUCAUCAAGGACA | | GSTIANDGSLDHEINARIQKASQALGRLRCKV | |
| | AAAAGGGAAUCAGCAACCGCUGGAAAGAACACUUUUCUCAACUUCUCAACCGC | | LQIIRGVSTATKLKVYNAVVLSSLLYGCETW | |
| | CCAAGCUCCGUGGACCAGAGCGCUCUGGACCAGAUCCCGCAGAACCGUACCAUC | | TLYRKHMKQLEQFHQRSLRSIMRIRWQDRIT | |
| | GAGCAGCUGGACGUGCCUCCCUCCAUCGAAGAGGUCCAGAAGGCUAUCAAGCA | | NQEVLDRANSTSIEVMVLKTQLRWSGHVIRM | |
| | GAUGUCUGCUGGGAAGGCUCCUGGCAAGGAUGGGAUCCCAACAGAGGUCUAUA | | DPQRIPRQVFYGELSAGLRKQGRPKKRFKDQ | |
| | AAGCUCUCAACGGAAAGGCGCUGCAGGCCUUUCACAUUGUCCUGACCUCCAUC | | LKSNLKWAGITPKQLELAASDRSSWRTHINH | |
| | UGGGAGGAAGAGGACAUGCCACCUGAACUUAGGGACGCCUCUAUUGUGGCCCU | | AATTFEDERRRRLAAARERRHQATTAPPVTT | |
| | GUACAAGAAUAAGGGCAGCCGCGCAGCUUGUGAUAACUAUCGUGGCAUCUCAC | | GVPCPMCHKLCASAFGLQSHMRVHRRSGSET | |
| | UCCUGAGUACAGCUGGCAAGAUUCUGGCUCGUGUGAUCCUGAACCGGUUGCUG | | PGTSESATPESVSGWRLFKKIS | |
| | AGUUCCGUUUCCGAGCAGAACCUGCCCGAAUCACAGUGCGGCUUCCGCCCCGAU | | | |
| | CGGUCCACCAUCGAUAUGGUGUUCACCGUGCGUCAGAUGCAGGAGAAGUGCCU | | | |
| | GGAACAGAAUCUGAGCCUGUACAUCGUUUUCAUCGAUCUGACUAAGGCCUUCG | | | |
| | ACACGGUGAACCGCGAUGCCCUGUGGGUGAUCCUGAGCAAGCUGGGGUGCCCC | | | |
| | GCUAAGUUCGUAAAGCUCAUCCAGUUGUUCCACGUUGAUAUGACGGGAGAGGU | | | |
| | CCUGUCCGGUGGAGAAACCUCCGACCGGUUUAACAUCUCCAAUGGCGUGAAAC | | | |
| | AGGGCUGCGUCCUGGCCCCAGUGCUGUUCAACCUGUUUUUCACACAGGUGCUG | | | |
| | AGACAUGCGGUUAUGGAUCUUGACCUGGGUGUGUAUAUCAAGUAUCGCUUGGA | | | |
| | CGGCAGUCUGUUCGAUCUCAGACGCCUGACAGCUAAGACUAAGACUACCGAGC | | | |
| | GCCUCAUUCUCGAAGCCCUGUUUGCCGACGAUUGUGCCCUUAUGGCCCACCAGG | | | |
| | AGAACCACCUGCAGACGAUUGUCGACCGCUUCUCUACAGCGACCAAGCUGUUC | | | |
| | GGCCUCACCAUCUCCCUCUCAAAGACCGAAGUCCUGUUUCAGCCCGCACCCGGG | | | |
| | AGACCCACGAAUCAGCCGUGCAUUACCAUUGACGGGACCCAACUUUCCAACGUC | | | |
| | AACACCUUCAAGUAUCUGGGAUCUACCAUCGCCAAUGAUGGCAGCCUGGAUCA | | | |
| | UGAAAUCAACGCAAGGAUCCAAAAGGCUAGCCAGGCACUGGGCCGCCUCCGGU | | | |
| | GCAAGGUCCUGCAGCACCGGGGCGUUUCCACUGCCACUAAACUGAAGGUGUAC | | | |
| | AACGCUGUCGUGCUCUCUUCCCUGCUUUAUGGCUGCGAAACAUGGACUCUGUA | | | |
| | CCGGAAGCAUAUGAAACAGCUUGAGCAGUUCCACCAGAGGAGCUUGCGUAGCA | | | |
| | UCAUGAGGAUCCGCUGGCAGGACCGCAUCACCAACCAGGAGGUGCUGGACAGG | | | |
| | GCUAAUUCCACCAGCAUCGAAGUUAUGGUGCUGAAGACCCAGCUCCGUUGGUC | | | |
| | UGGCCAUGUCAUUCGGAUGGACCCUCAGCGUAUCCCCCGCCAGGUAUUCUACG | | | |
| | GGGAACUGUCUGCGGGGCUCAGAAAGCAGGGCCGCCCAAAGAAACGCUUUAAG | | | |
| | GAUCAGCUGAAAAGCAAUCUGAAGUGGGCCGGCAUCACCCCCAAGCAGCUGGA | | | |
| | GCUGGCGGCAUCUGACCGCUCCAGCUGGCGCACCCACAUUAACCACGCAGCUAC | | | |
| | UACCUUUGAGGACGAACGCCGGCGCAGACUGGCCGCUGCACGCGAGAGGCGCC | | | |
| | ACCAAGCCACUACCGCCCCUCCCGUGACCACUGGGGUCCCUUGCCCUAUGUGCC | | | |
| | ACAAGCUGUGUGCGUCUGCCUUCGGUUUGCAGUCCCACAUGCGUGUGCAUCGC | | | |
| | CGCAGCGGCAGCGAGACUCCCGGGACCUCAGAGUCCGCCACACCCGAAAGUGUG | | | |
| | AGCGGCUGGCGGCUGUUCAAGAAGAUUAGCUGAGCUGGAGCCUCGGUGGCCAU | | | |
| | GCUUCUUGCCCCUUGGGCCUCCCCCCAGCCCCUCCUCCCCUUCCUGCACCCGUA | | | |
| | CCCCCGUGGUCUUUGAAUAAAGUCUGAAAAAAAAAAAAAAAAAAAAAAAAAAA | | | |
| | AAAAAAAAAAAAAAAAAAAAAAAAAAAAAAAAAAAAAAAAAAAAAAAAAAAA | | | |
| | AA | | | |
| Vingi- | AGGAAAUAAGAGAGAAAAGAAGAGUAAGAAGAAAUAUAAGAGCCACCAUGGAC | 15,014 | MDEYQRSLSRPLLTIMSINIEGLSLAKEELLAK | 15,002 |
| 1_Acar | GAGUACCAGCGCUCCUUGUCCCGCCCCCUGUUGACAAUCAUGAGUAUUAAUAU | | MSEDISCDILCIQETHRDITMRRPKILGMQLA | |
| RNAIVT | CGAAGGCUUGAGCCUCGCGAAAGAGGAACUGCUUGCCAAGAUGUCCGAGGACA | | VERPHRQYGSAIFVRSGVAISATSLTEVNNIEI | |
| 696 | UCAGCUGCGACAUUCUGUGCAUCCAAGAGACCCAUCGCGACAUCACCAUGCGUC | | LSVELDSCTVSSLYKPPGADFYFTPPTSCIINII | |
| | GCCCCAAGAUCCUCGGUAUGCAGCUUGCGGUGGAGCGCCCUCACCGCCAGUACG | | EAHFVVGDFNSHSCVWGYDEDDRNGEAVLT | |
| | GCUCCGCGAUUUUCGUGCGUUCCGGUGUGGCCAUUUCUGCUACCAGCCUCACCG | | WADNSRMSLLHDSKLPPSFNSGRWKRGYNP | |
| | AGGUUAACAAUAUUGAGAUCCUGUCAGUGGAGCUCGAUUCCUGUACCGUCUCC | | DLIFVKESISHQCTKRVLNPIPNTQHRPICCVA | |
| | AGUCUGUACAAACCUCCCGGUGCAGACUUCUACUUUACUCCCCCGACGUCUUGC | | YAAVRPKSVPFRRRYNFNKANWTKFTETLEA | |
| | CACAACCAUGAGGCUCAUUUUGUGGUUGGUGAUUUCAAUUCCCACUCCUGCGU | | AISDIEPSIENYDLFVEAVKRSSRLSIPRGCRTS | |
| | GUGGGGCUAUGACGAGGAUGACCGCAACGGCGAAGCUGUUCUGACCUGGGCCG | | YLPGLNEESLNQLQEYLRLFQENPYSDGTIAA | |
| | AUAAUUCCCGCAUGUCCCUGCUCCACGAUAGUAAGCUGCCUCCCUCCUUCAACA | | GQKLSTALANAKKDRWIELLENLDMSKSSRK | |
| | GUGGGAGAUGGAAGCGCGGGUACAACCCUGAUCUUAUCUUCGUCAAAGAAUCC | | AWQLLRRLDSDPLVNPGHANVTPDQIAHQLI | |
| | AUCUCUCAUCAGUGUACCAAGCGCGUCCUCAACCCGAUUCCUAACACUCAGCAC | | QNGKTNCSRIKMKINRVPELETHQLSSPLNLK | |
| | CGCCCCAUUUGUUGCGUAGCAUAUGCUGCCGUGCGUCCCAAGUCUGUCCCCUUC | | ELREAIKRCKTGKAPGLDDLMMEQIKHLGAK | |
| | AGAAGGAGAUACAACUUCAACAAGGCUAACUGGACCAAGUUUACCGAAACCUU | | AENWLLKFYNQCLAHKQIPRAWRKTKIIAIL | |
| | GGAAGCGGCAAUCUCCGACAUUGAGCCAUCUAUCGAGAAUUAUGAUCUCUUCG | | KPGKDASNARNYRPISLLCHLYKVYERMLLN | |
| | UUGAGGCAGUCAAGCGGUCCUCUCGCCUGAGUAUCCCCCGUGGAUGUAGGACC | | RLGPVIEPKLIAQQAGFRPGKNCTGQILHLTE | |
| | UCCUACCUGCCCGGCCUGAACGAAGAGUCACUCAAUCAGCUGCAGGAAUACCU | | HIEEGYEKGCITGTVFVDLTAAYDTVQHRKM | |
| | GCGCCUGUUCCAAGAGAAUCCUUACUCCGAUGGCACCAUCGCUGCCGGACAAA | | LHKVYHITRDFDFTKTVQTLLENRSFYVEFQG | |
| | AACUGAGCACCGCUCUGGCGAAUGCUAAGAAAGACCGCUGGAUCGAGCUGCUC | | QKSRWRRQKNGLPQGSVLAPTLFNIFTNDQP | |
| | GAGAACCUGGACAUGUCCAAGUCUAGCCGGAAAGCUUGGCAGCUCCUGCGCCG | | QPPLTKSFIYADDLGLTTQAKDFETVEKQLTN | |
| | GCUGGACAGCGACCCCCUCGUGAAUCCUGGCCAUGCCAAUGUAACGCCGGACCA | | ALKDLSSYYKENHLKPNPAKTQVCAFHLRNR | |
| | GAUCGCCCAUCAACUGAUCCAGAACGGCAAGACCAAUUGCAGUAGAAUCAAGA | | EANRKLKVTWEGQELEHCFHPKYLGVTLDR | |
| | UGAAGAUCAACAGAGUACCCGAGUUGGAAACACACCAGCUGUCUAGUCCUCUC | | TLTYRKHCMNTKHKVAARNNILRKLTGSAW | |
| | AACCUCAAAGAGCUCCGCGAAGCAAUCAAAAGGUGUAAGACGGGCAAAGCUCC | | GADPQVIRTSALALSFSTAEYACPVWHKSAH | |
| | CGGUCUGGAUGACCUGAUGAUGGAACAGAUCAAACACUUGGGGGCCAAGGCUG | | AKQVDIALNETCRIITGCLKPTPVDKLYKLAG | |
| | AGAACUGGUUGCUGAAAUUCUACAACCAGUGCCUGGCUCACAAGCAGAUCCCC | | IAPPDVRREVAANGERKKVEHCESHPLHGYH | |
| | CGUGCGUGGCGCAAGACUAAGAUUAUCGCCAUCUUGAAGCCGGGCAAGGAUGC | | PPPTRLKSRKGFMRTTTPLDVPPAAARVSLW | |
| | UAGUAACGCCCGCAACUACCGUCCUAUUUCCCUCCUGUGUCACCUUUACAAGGU | | AAKPGNSNWMAPQEGLPPGANQEWATWKS | |
| | GUACGAGCGCAUGCUCCUGAACCGCUUGGGCCCCGUCAUCGAGCCUAAACUGA | | LNRLRSGVGRSKDNLARWHYLEESSTLCDCG | |
| | UCGCCCAACAGGCUGGUUUCCGCCCAGGUAAGAACUGCACUGGACAGAUCCUG | | AEQTTQHMYACPQCPASCTEEELFKATDNAV | |
| | CACCUUACGGAGCACAUCGAGGAAGGUUACGAGAAGGGCUGCAUCACCGGAAC | | AVARFWSKTIGGGSPKKKRKVSGSETPGTSES | |
| | CGUCUUCGUGGACCUGACUGCGGCCUACGACACGGUUCAGCACAGAAAAAUGC | | ATPESVSGWRLFKKIS | |
| | UCCACAAGGUAUAUCAUAUUACCCGGGAUUUCGACUUCACCAAAACAGUGCAG | | | |
| | ACGCUUCUGGAGAAUCGCUCCUUUUACGUCGAGUUCCAGGGUCAGAAAAGUCG | | | |
| | GUGGAGGCGCCAGAAGAACGGCUUGCCCCAGGGUAGCGUGCUCGCCCCAACCCU | | | |
| | GUUUAAUAUCUUCACCAACGACCAGCCACAGCCCCCGCUGACAAAAUCCUUUAU | | | |
| | CUAUGCCGACGAUCUUGGCCUGACCACGCAAGCCAAGGACUUUGAGACAGUCG | | | |
| | AGAAACAGCUCACAAACGCACUCAAGGACCUGAGUUCCUAUUACAAGGAGAAC | | | |
| | CAUCUCAAACCUAACCCCGCUAAGACACAGGUCUGCGCUUUUCACCUGAGGAA | | | |
| | UCGGGAGGCCAACCGUAAACUGAAGGUGACAUGGGAGGGACAGGAACUGGAGC | | | |
| | ACUGUUUCCACCCCAAGUAUUUGGGCGUGACUCUCGAUCGCACCCUUACGUAU | | | |
| | CGGAAGCACUGCAUGAACACUAAGCAUAAGGUGGCGGCCAGAAAUAACAUCCU | | | |
| | GCGUAAACUCACAGGAAGCGCCUGGGGCGCCGACCCACAGGUCAUCCGUACCAG | | | |
| | UGCUCUGGCCCUGUCUUUCUCCACUGCUGAAUAUGCGUGUCCGGUGUGGCACA | | | |
| | AGUCCGCCCACGCGAAGCAGGUGGAUAUUGCCCUGAACGAGACAUGCCGGAUU | | | |
| | AUCACUGGUUGCCUCAAGCCUACCCCCGUCGACAAGCUCUAUAAGUUGGCCGGC | | | |
| | AUUGCACCCCCUGAUGUCCGUCGCGAAGUCGCAGCCAAUGGCGAGCGGAAAAA | | | |
| | GGUAGAACACUGCGAAUCACAUCCGCUGCAUGGAUAUCAUCCCCCUCCCACCCG | | | |
| | GCUUAAGAGCCGUAAGGGCUUCAUGCGCACAACCACUCCGCUGGAUGUGCCUC | | | |
| | CCGCUGCCGCGCGCGUGAGUCUGUGGGCCGCUAAGCCCGGUAAUUCCAAUUGG | | | |
| | AUGGCACCGCAAGAGGGCCUCCCGCCCGGAGCCAAUCAAGAGUGGGCCACAUG | | | |
| | GAAGUCCUUGAACAGGCUGCGCUCCGGUGUGGGACGCUCUAAGGACAACCUGG | | | |
| | CCCGUUGGCACUAUCUGGAAGAGUCAUCCACCCUGUGCGACUGUGGUGCUGAA | | | |
| | AGACUCCCGGGACCUCAGAGUCCGCCACACCCGAAAGUGUGAGCGGCUGGCGGC | | | |
| | UGUUCAAGAAGAUUAGCUGAUUAAUUAAGCUGGAGCCUCGGUGGCCAUGCUUC | | | |
| | UUGCCCCUUGGGCCUCCCCCCAGCCCCUCCUCCCCUUCCUGCACCCGUACCCCCG | | | |
| | UGGUCUUUGAAUAAAGUCUGAUCUAGAAAAAAAAAAAAAAAAAAAAAAAAAA | | | |
| | AAAAAAAAAAAAAAAAAAAAAAAAAAAAAAAAAAAAAAAAAAAAAAAAAAAA | | | |
| | AA | | | |
| CR1- | AGGAAAUAAGAGAGAAAAGAAGAGUAAGAAGAAAUAUAAGAGCCACCAUGCUG | 15,015 | MLYTIFLVYGILCFFFVYFCIYLYITVYLCFLFF | 15517 |
| 1_PH | UAUACCAUCUUCCUCGUUUAUGGGAUCCUGUGUUUUUUCUUUGUGUAUUUCUG | | LFLLLCGDVESNPGPGRARGCRLLYCNIRGLH | |
| RNAIVT | CAUCUACCUGUACAUCACUGUUUAUCUUUGUUUUCUGUUCUUUCUGUUUCUGC | | ANLAELDFVSRGVDVVCCSETLVAGRRHDA | |
| 636 | UUCUGUGCGGUGACGUGGAAAGCAAUCCUGGACCAGGCAGAGCCCGUGGAUGU | | ELALAGFQSPFRRLCGSGPGFRGMAVYVRSG | |
| | CGGUUGCUGUAUUGCAACAUCCGCGGUCUGCAUGCUAAUCUGGCCGAGCUGGA | | FCAYRQSVHECACHEIIVVRVCGRLNNYYLF | |
| | CUUUGUUAGUCGCGGGGUGGACGUGGUCUGCUGUUCCGAGACUCUGGUGGCUG | | SLYRSPATDDSLYDCLLTAMASIQSTDPKAAF | |
| | GGCGUCGCCACGAUGCCGAGUUGGCCCUCGCUGGCUUCCAGUCCCCCUUUCGUA | | VFVGDVNAHHRDWLGSASPTDCHGVAALDF | |
| | GGCUCUGUGGCUCAGGCCCUGGCUUCCGUGGCAUGGCGGUGUACGUCAGGUCU | | CTLSSCVQLVRGSTHIAGNCLDLVMTDVPDL | |
| | GGGUUCUGUGCCUACCGGCAGAGUGUGCACGAAUGUGCCUGCCACGAGAUUAU | | MTVTVGSPIGSSDHSHLVVSLDLNQVVPVVD | |
| | CGUCGUUCGCGUGUGUGGACGUCUGAACAAUUACUAUCUGUUCUCUCUCUACC | | TRRVVFVKSRANWVAITRAVRTLPWRQIIHS | |
| | GCUCCCCGGCCACCGAUGACUCCCUUUACGAUUGCCUUCUGACUGCCAUGGCCA | | EDPVSELNDLTVSILERFVPKRTILVRSRDKP | |
| | GCAUCCAGUCCACUGAUCCGAAGGCAGCCUUCGUCUUCGUUGGCGACGUAAAC | | WFDDQCRLAFEAKQAAYRAWRRSRDRTLW | |
| | GCACAUCACCGGGACUGGCUGGGAAGCGCCUCUCCAACAGAUUGUCACGGCGU | | QTYVDRRAEAKRVYEEAQRRLRQRSRESLLS | |
| | CGCCGCUCUGGACUUUUGCACCCUUUCCAGCUGCGUGCAACUCGUCCGUGGGUC | | IDHPHRWWSELKGSVFGAEPSLPPLVGPGGGI | |
| | CACUCAUAUCGCUGGAAACUGUCUGGAUCUGGUGAUGACCGACGUGCCUGACC | | ITDPLARAELLSAHFDGKQSRDVIALPHGCHP | |
| | UGAUGACCGUGACCGUCGGAUCCCCGAUUGGAAGUAGCGACCACUCCCACCUG | | EPRLTSLAFRSGAIKVLLEGLDPYGGVDPVG | |
| | GUGGUCUCACUGGAUCUCAACCAGGUCGUGCCUGUUGUGGAUACACGCAGGGU | | MFPLFYKQLADVLAPKLAVIFRRLIRLGNFPR | |
| | UGUGUUCGUGAAAAGCCGCGCCAACUGGGUCGCCAUCACUCGCGCUGUGAGGA | | CWRTGNITPIPKGPVSPYVANYRPITLTPILSK | |
| | CUCUGCCUUGGCGGCAGAUUAUCCACUCCGAGGACCCUGUAAGUGAGCUCAAU | | VFEKLIAGKLGRFAEVTGLLPAGQFAYRIGLG | |
| | GACCUGACCGUGUCUAUUCUGGAACGCUUUGUUCCCAAGCGCACCAUCCUCGU | | CCDALLSVSHHLQSALDCRSEARLVQLDFSA | |
| | GAGAAGCCGCGAUAAGCCUUGGUUCGAUGACCAGUGUAGACUGGCUUUCGAGG | | AFDRVNHRGLLYKLESFGVGGRVLSIIRDFVS | |
| | CUAAGCAGGCGGCUUACCGUGCUUGGCGUAGGAGUCGGGACCGCACGUUGUGG | | ERTQSVSVDGVLSASVGVVSGVPQGSVLGPL | |
| | CAGACCUAUGUGGACAGGCGGGCCGAGGCCAAGCGCGUGUAUGAAGAGGCUCA | | LFVLYTSDMFSSLENTLINYADDSTLMAVIPA | |
| | GAGGCGCCUUCGCCAGAGGAGCCGUGAAAGCCUGCUCAGUAUUGACCACCCUC | | PRLRDAVAQSLNRDLSRISAWCSAWSMKLN | |
| | AUCGCUGGUGGUCAGAGCUCAAGGGCUCCGUGUUCGGCGCCGAACCCAGCCUG | | ASKTKSMIISRSRTLVPQHPQLEIDDTLLQESS | |
| | CCCCCUCUCGUUGGUCCUGGCGGUGGAAUCAUUACCGAUCCCCUCGCCAGGGCC | | SLEILGVVFDEKLTFEPHIRRLVSRASTKIGLL | |
| | GAGCUGCUCUCCGCACAUUUUGAUGGAAAGCAGUCACGCGAUGUCAUCGCCUU | | RKVNSVFGDSQVARRCFYAFLLPVLEYCSPV | |
| | GCCCCACGGUUGCCACCCGGAGCCCCGCCUCACAUCUCUGGCCUUCAGGUCCGG | | WASAADTHLRLLDRLVSSASRLCADNDLVN | |
| | CGCUAUCAAGGUCCUCCUGGAAGGACUCGACCCCUACGGUGGCGUUGACCCCGU | | LSIIRRRVAELCMFYKVYNNERIIWLYSSLPA | |
| | CGGCAUGUUUCCUCUGUUUUACAAGCAGCUGGCGGACGUCCUGGCCCCAAAGU | | LKVFGRETRAACGAHSFTLEAVRCRTNQFCR | |
| | UGGCCGUUAUCUUUCGUCGGCUGAUCCGCCUCGGCAACUUUCCCCGCUGCUGGC | | CFVPWSAKVWNLLPASAFGRIGLQAFKSAVN | |
| | GCACUGGUAAUAUCACUCCCAUCCCCAAGGGCCCCGUGAGUCCUUACGUGGCCA | | GFLLDFLGGGSPKKKRKVSGSETPGTSESATP | |
| | AUUACCGUCCCAUCACCUUGACACCUAUCCUGUCUAAGGUUUUCGAAAAGCUG | | ESVSGWRLFKKIS | |
| | AUCGCAGGCAAGCUGGGUCGCUUUGCUGAAGUCACUGGCUUGCUCCCUGCUGG | | | |
| | UCAGUUUGCUUAUCGCAUCGGUCUGGGCUGCUGUGACGCUCUCCUGUCCGUGA | | | |
| | GCCAUCACCUGCAGUCCGCCCUGGACUGCCGCUCCGAAGCCCGCCUGGUGCAGC | | | |
| | UCGAUUUCUCCGCAGCGUUUGACCGCGUUAAUCACAGGGGUCUUCUGUAUAAA | | | |
| | UUGGAGUCCUUCGGCGUCGGGGGCCGGGUGCUGUCAACUAUCCGUGAUUUCGU | | | |
| | AUCCGAGCGCACCCAGUCUGUAUCCGUGGACGGUGUUCUGUCCGCCAGCGUCG | | | |
| | GCGUGGUUAGCGGAGUCCCCCAGGGUAGCGUGCUGGGACCUCUGCUCUUUGUA | | | |
| | UUGUAUACGUCCGACAUGUUCUCUAGCCUGGAGAAUACCCUCAUUAACUAUGC | | | |
| | AGAUGACUCCACUUUGAUGGCAGUUAUUCCGGCCCCACGCCUGCGUGACGCCG | | | |
| | UAGCGCAGUCCCUGAAUAGGGACCUGUCACGCAUCAGUGCCUGGUGUUCAGCU | | | |
| | UGGUCCAUGAAGCUGAACGCGUCCAAGACCAAAAGCAUGAUUAUCUCAAGGUC | | | |
| | CAGGACUUUGGUGCCUCAACACCCCCAGCUGGAGAUCGACGAUACUUUGCUGC | | | |
| | AGGAGUCCUCAUCCCUGGAAAUCCUGGGCGUCGUGUUCGAUGAGAAGCUCACC | | | |
| | UUCGAGCCUCACAUCAGGCGGCUGGUGAGCCGCGCGUCUACCAAGAUCGGGCU | | | |
| | GCUCCGCAAGGUGAACUCCGUGUUCGGCGAUUCCCAGGUCGCGAGGCGUUGCU | | | |
| | UUUACGCUUUCCUGCUCCCUGUGCUGGAGUACUGCUCUCCGGUAUGGGCGAGU | | | |
| | GCCGCGGAUACCCAUCUUCGUCUUCUGGACCGCCUUGUCUCCAGCGCCAGCCGU | | | |
| | UUGUGUGCUGACAACGAUCUGGUGAACCUGAGUCACCGUCGGCGCGUGGCUGA | | | |
| | GCUGUGUAUGUUCUACAAGGUGUACAAUAACGAGAGGCAUUGGCUGUACUCAU | | | |
| | CCCUGCCAGCCCUGAAGGUCUUCGGGCGCGAAACGCGUGCCGCGUGCGGUGCUC | | | |
| | ACUCUUUUACGUUGGAGGCGGUUCGCUGUCGCACCAACCAGUUCUGCCGCUGC | | | |
| | UUUGUGCCUUGGUCUGCGAAGGUGUGGAACCUGCUCCCUGCCUCCGCUUUCGG | | | |
| | GAGAAUCGGUCUGCAGGCAUUUAAAAGCGCUGUGAACGGUUUCCUGUUGGACU | | | |
| | UUCUGGGCGGCGGCUCCCCCAAGAAGAAGCGGAAGGUGAGCGGCAGCGAGACU | | | |
| | CCCGGGACCUCAGAGUCCGCCACACCCGAAAGUGUGAGCGGCUGGCGGCUGUUC | | | |
| | AAGAAGAUUAGCUGAUUAAUUAAGCUGCCUUCUGCGGGGCUUGCCUUCUGGCC | | | |
| | AUGCCCUUCUUCUCUCCCUUGCACCUGUACCUCUUGGUCUUUGAAUAAAGCCU | | | |
| | GAGUAGGAAGUCUAGAAAAAAAAAAAAAAAAAAAAAAAAAAAAAAAAAAAAA | | | |
| | AAAAAAAAAAAAAAAAAAAAAAAAAAAAAAAAAAAAAAAAAAA | | | |
| RTE- | AGGAAAUAAGAGAGAAAAGAAGAGUAAGAAGAAAUAUAAGAGCCACCAUGCCA | 15,016 | MPKKKRKVGGSSGTPRVAFDSGKDLRNPIGQ | 15,017 |
| 3_BF | AAGAAGAAAAGAAAAGUAGGAGGUUCCAGCGGCACACCAAGAGUGGCAUUUGA | | SPPALSRAAPGQLGTDPSRSACFIGCLELRVLL | |
| RNAIVT | CAGCGGAAAGGACCUCAGAAAUCCCAUAGGGCAGUCCCCACCUGCCCUCAGCCG | | DKWIVCRAPDKQESKEKRRQKTQPIRIGSWN | |
| 1770 | GGCUGCCCCCGGACAGUUGGGGACAGACCCCUCACGUUCCGCAUGUUUUAUAG | | VRTMRTGLSDDLTVIEDIRKTAAIDRELYRLN | |
| | GGUGCCUGGAGCUUCGAGUCCUGUUGGACAAAUGGAUCGUGUGCCGAGCUCCU | | IDIVALQETRLPDSGSLKEDSYTFFWQGKGME | |
| | GAUAAGCAGGAAUCCAAGGAGAAACGCCGCCAGAAAACUCAGCCUAUACGGAU | | ETREHGVGFAVRNTLLHMIEPPTGGTERIITLR | |
| | UGGAAGCUGGAACGUCAGAACAAUGCGGACAGGCCUGUCUGAUGACUUGACAG | | LSTHEGPVNLLCVYAPTLQATSEVKDQFYGQ | |
| | UUAUCGAGGACAUCCGUAAAACAGCCGCAAUUGACCGCGAACUUUAUCGCCUG | | LDSAIKKIPVSEHIFILGDFNARVGTDQESWQT | |
| | AAUAUUGACAUUGUUGCGCUGCAAGAAACGCGUUUGCCCGACAGCGGUUCUCU | | VLGHHGIGKMNENGQRLLELCCYHNLCVTN | |
| | CAAGGAAGACAGCUAUACUUUCUUCUGGCAGGGUAAGGGUAUGGAGGAAACAA | | TFFQNKAIHKASWRHPRSQRWHQLDLVITRR | |
| | GGGAGCACGGCGUGGGGUUUGCCGUGAGAAACACUUUGCUCCACAUGAUUGAG | | TSLNSVCNTRAYIISADCDTDIISLIAARIKLRP | |
| | CCUCCCACGGGUGGUACCGAGCGCAUCAUUACACUUCGUUUAAGUACCCAUGA | | KKLHHMKKKGQPKIDVSKTMLPDRNQKFLE | |
| | GGGCCCUGUGAAUCUCCUCUGUGUUUACGCUCCCACCCUGCAGGCGACAUCAGA | | CLEGTLNNIQPQDAEHRWETLSKTIYSAAAQS | |
| | GGUCAAAGAUCAAUUUUACGGGCAGUUAGACUCCGCUAUCAAGAAGAUACCCG | | YGKKERKNTDWFEAYISELEPVMDTKRKAL | |
| | UCAGCGAGCACAUUUUUAUCUUGGGAGACUUUAACGCCCGGGUCGGGACGGAC | | VSYKQNPSSQNLQALKAARQEAQRASRRCA | |
| | CAGGAGUCAUGGCAGACCGUGCUCGGACAUCACGGGAUUGGGAAAAUGAACGA | | NNYWLLLSERIQLASATGDIRRMYEGIKQAT | |
| | AAACGGGCAGCGGCUGCUCGAACUAUGUUGCUAUCACAACCUGUGCGUCACCA | | GKPIKKSAPLKAKSGEIITDKDKQMARWVEH | |
| | AUACCUUCUUCCAGAAUAAAGCCAUUCAUAAAGCUAGUUGGAGACAUCCCAGA | | YLDIYSTENSVSQDALDNIEDFSVLAELDADP | |
| | AGUCAACGAUGGCACCAGCUAGACUUGGUAAUCACCAGGCGGACAAGCCUCAA | | TIEELSKAIDSMSNGKAPGEDNIPAELIKSGKS | |
| | UUCCGUUUGCAAUACCAGGGCCUAUCAUUCCGCGGACUGUGAUACUGAUCACA | | VLLEPLHELLRLCWKEGKVPQSMRNSKIVTL | |
| | GCCUGAUCGCUGCGAGAAUCAAGCUGAGGCCUAAGAAACUGCACCAUAUGAAG | | YKNKGDRTDCNSYRGISLLSIVGKVFAKVVL | |
| | AAGAAAGGCCAGCCAAAGAUCGAUGUGUCUAAAACUAUGCUCCCCGACAGGAA | | TRLQVLADRVYPESQCGFRAERSTTDMIFSVR | |
| | CCAGAAGUUCCUUGAAUGCCUGGAAGGCACACUCAACAACAUCCAACCGCAGG | | QLQEKCREQQRPLYIAFIDLTKAFDLVSRRGL | |
| | AUGCAGAGCACAGGUGGGAGACCCUGUCCAAGACCAUCUACAGCGCCGCCGCAC | | FQLLRKIGCPPQLLDIIISFHEDMKGVVSEDGE | |
| | AGUCCUAUGGCAAGAAGGAGCGGAAGAACACAGACUGGUUCGAGGCUUAUAUC | | TSEPFAIRSGVKQGCVLAPTLFGIFFSLLLKSA | |
| | UCUGAGCUUGAGCCUGUGAUGGAUACGAAGAGGAAAGCCCUGGUGAGUUAUAA | | FGHSTQGVHLHTRSDGKLFNLARLRAKTKVR | |
| | GCAGAAUCCCAGCUCACAAAAUCUGCAGGCCCUGAAGGCUGCCCGCCAGGAAGC | | SVLIRDMLFADDAALVAHVEDELQQLLNQFA | |
| | UCAGAGAGCCUCACGACGAUGCGCCAACAACUACUGGUUGCUACUGUCUGAGA | | HACSEFALTISIKKTVVMGQDVPQPPVVTIGS | |
| | GAAUUCAACUAGCGUCCGCGACCGGCGAUAUUCGCCGCAUGUAUGAGGGCAUC | | EVLEVTDHFTYLGSTVTSNLSLDKEIDRRIAR | |
| | AAGCAAGCCACCGGUAAACCUAUCAAAAAAUCUGCCCCCCUUAAGGCUAAAUC | | AAGVMTKLGTRVWNNSHLTLNTKLEVYRSC | |
| | CGGCGAGAUUAUAACCGAUAAGGACAAGCAAAUGGCUCGCUGGGUCGAACACU | | VLSTLLYGSETWTTYAKQENRLESFHLRCLR | |
| | ACCUGGAUAUUUAUUCUACAGAGAACUCUGUCUCUCAGGAUGCCCUGGAUAAC | | RILGISWRDRVPNTTVLERSCSLSIHLLLCQRR | |
| | AUCGAGGAUUUUUCUGUGCUGGCCGAACUCGAUGCUGAUCCAACCAUAGAAGA | | LRWLGHVSRMKDGRIPKDILFGELATGKRPV | |
| | GUUAUCUAAGGCUAUAGACUCAAUGUCAAACGGCAAAGCACCAGGCGAAGACA | | GRPALRFRDVCKRDLKLTDIDPASWEQIAAD | |
| | AUAUUCCAGCCGAAAUUAUAAAAAGUGGUAAAUCUGUGCUCCUUGAACCGCUG | | RNRWRHTVKDGLAKGQERRTEHLESRRRKR | |
| | CAUGAAUUACUGAGACUGUGUUGGAAAGAGGGGAAAGUCCCGCAGUCAAUGCG | | KEKPQQGNPSAFICPNCGRDCHARIGLQSHSR | |
| | AAACUCGAAAAUCGUAACUCUGUACAAGAAUAAAGGAGAUAGGACUGACUGCA | | RCQPPSGSETPGTSESATPESVSGWRLFKKIS | |
| | AUUCCUACCGCGGCAUAAGCUUGUUGUCUAUCGUGGGCAAAGUAUUCGCUAAA | | | |
| | GUGGUGCUAACCAGGCUGCAGGUGCUGGCUGAUCGCGUGUACCCAGAGAGCCA | | | |
| | GUGUGGCUUCCGAGCCGAGAGGAGUACCACUGACAUGAUCUUUUCAGUGCGGC | | | |
| | AGCUUCAGGAGAAGUGCCGCGAGCAACAACGGCCUCUGUACAUCGCCUUUAUU | | | |
| | GACUUGACUAAGGCAUUCGACUUAGUGAGUAGGAGGGGACUUUUCCAGCUCCU | | | |
| | GAGGAAGAUUGGUUGUCCUCCACAACUGCUGGAUAUAAUUAUCUCGUUCCAUG | | | |
| | AGGAUAUGAAGGGAGUCGUUAGUUUCGAUGGGGAAACAAGCGAACCAUUCGCA | | | |
| | AUUAGGUCUGGCGUCAAGCAAGGCUGCGUUCUGGCACCUACGUUGUUUGGCAU | | | |
| | CUUUUUCUCCCUCCUCCUGAAGUCCGCAUUUGGGCACAGCACUCAGGGUGUGC | | | |
| | ACCUGCAUACCCGGUCUGAUGGGAAGCUUUUUAACCUCGCCCGCCUCAGGGCCA | | | |
| | AGACUAAGGUACGCUCUGUAUUAAUCAGAGAUAUGCUGUUCGCAGACGACGCA | | | |
| | GCGCUCGUUGCUCAUGUUGAGGACGAGCUUCAACAGUUAUUGAAUCAGUUCGC | | | |
| | ACAUGCAUGCUCAGAAUUCGCCCUCACAAUUAGUAUCAAAAAAACUGUCGUGA | | | |
| | UGGGUCAGGAUGUGCCACAGCCCCCAGUGGUGACGAUCGGAAGUGAGGUCCUG | | | |
| | GAGGUUACCGACCACUUUACGUACCUUGGAAGCACUGUGACCUCCAACCUGUC | | | |
| | CCUAGACAAAGAAAUCGACAGGCGCAUUGCCCGGGCAGCCGGAGUCAUGACAA | | | |
| | AACUGGGAACCCGAGUGUGGAAUAAUAGUCACCUCACGCUGAAUACUAAGCUU | | | |
| | GAAGUGUAUAGAUCGUGUGUUCUGAGCACACUGCUUUACGGGUCCGAAACCUG | | | |
| | GACCACCUACGCUAAGCAGGAGAAUAGACUGGAGAGCUUUCACCUUAGGUGUC | | | |
| | UUCGUCGAAUCCUAGGCAUUUCCUGGAGAGAUCGGGUUCCCAACACAACCGUG | | | |
| | UUGGAGAGAAGCUGCUCACUGAGUAUUCACCUCUUACUGUGCCAGCGGCGGCU | | | |
| | GCGGUGGCUGGGCCACGUAAGCAGGAUGAAGGACGGACGUAUCCCGAAAGAUA | | | |
| | UUCUCUUCGGGGAACUGGCUACUGGAAAGCGUCCCGUCGGCCGCCCAGCUCUCC | | | |
| | GCUUCAGGGACGUAUGUAAAAGAGAUUUGAAACUAACUGAUAUCGAUCCGGCC | | | |
| | UCCUGGGAACAGAUCGCAGCCGACCGAAACCGUUGGCGCCACACUGUGAAGGA | | | |
| | CGGACUGGCAAAAGGGCAAGAAAGACGGACAGAGCACUUAGAAUCACGGCGGC | | | |
| | GAAAGAGAAAGGAGAAGCCUCAGCAGGGUAACCCAAGUGCCUUUAUCUGUCCU | | | |
| | AAUUGCGGCAGGGACUGCCAUGCGCGGAUAGGACUGCAAUCGCAUUCAAGAAG | | | |
| | AUGUCAGCCCCCUAGCGGCAGCGAGACUCCCGGGACCUCAGAGUCCGCCACACC | | | |
| | CGAAAGUGUGAGCGGCUGGCGGCUGUUCAAGAAGAUUAGCUGAGCUGGAGCCU | | | |
| | CGGUGGCCAUGCUUCUUGCCCCUUGGGCCUCCCCCCAGCCCCUCCUCCCCUUCC | | | |
| | UGCACCCGUACCCCCGUGGUCUUUGAAUAAAGUCUGAAAAAAAAAAAAAAAAA | | | |
| | AAAAAAAAAAAAAAAAAAAAAAAAAAAAAAAAAAAAAAAAAAAAAAAAAAAA | | | |
| | AAAAAAAAAAAA | | | |
| RTE- | AGGAAAUAAGAGAGAAAAGAAGAGUAAGAAGAAAUAUAAGAGCCACCAUGCCC | 15,018 | MPRKNWNCGRRTGDEKRKMMFGCWNVQGI | 15519 |
| 25_LMi | CGCAAAAAUUGGAACUGCGGGCGACGAACCGGGGAUGAAAAACGUAAGAUGAU | | STKIDLLPAELDMFNIDVVVLSETKRKGKGEE | |
| RNAIVT | GUUUGGAUGCUGGAAUGUGCAGGGAAUCUCGACAAAGAUAGAUCUGCUGCCAG | | ELDNYVHIWSGVSKAVRAKAGVSIMIQKKW | |
| 1772 | CAGAACUGGACAUGUUCAACAUUGAUGUCGUGGUGCUCUCCGAGACAAAGAGG | | KKRITNWTFINERIITVEMTLFAREVVIIGVYA | |
| | AAGGGCAAGGGUGAAGAGGAGUUGGAUAAUUACGUUCACAUCUGGUCAGGGGU | | PTNDTKDKEKDAFWDTLRETIEKIPRRKELII | |
| | CUCUAAGGCUGUCAGAGCCAAGGCUGGUGUCUCCAUCAUGAUACAGAAAAAGU | | MGDMNGRVGIRESCKIVGKHGEAEYNDNGE | |
| | GGAAGAAACGUAUCACUAACUGGACGUUCAUAAAUGAGCGCAUCAUAACCGUU | | RLIDICAQFDLKITNTFFKHKDIHKYTWQQNT | |
| | GAAAUGACACUGUUUGCGCGGGAAGUUGUUAUCAUCGGCGUAUACGCCCCCAC | | KELRSIIDYIIIRQTSSFKAADVRSYRGAQCGS | |
| | CAACGACACUAAGGAUAAAGAGAAAGACGCGUUUUGGGACACUCUCAGAGAAA | | DHYLVKMKSFWPWKNATNDTSNINKMNCSE | |
| | CCAUCGAAAAAAUCCCCAGAAGGAAGGAACUUAUCAUAAUGGGUGAUAUGAAU | | KVQNVHFNIDSLQDESIRTFFKARMERTLDES | |
| | GGCCGGGUGGGGAUUAGAGAAUCCUGCAAGAUAGUGGGCAAGCAUGGAGAAGC | | FEGSTEEIYEYIKTKVKNVASEVLGIKENNPK | |
| | AGAGUAUAACGACAAUGGAGAGCGUCUUAUUGAUAUUUGCGCCCAGUUCGACU | | RAAEWWSEEIETSVKEKRNAFVQWLNDKSE | |
| | UGAAGAUCACUAACACGUUUUUCAAGCACAAGGACAUCCACAAGUAUACAUGG | | GTRSKYKEKKNEVEKKIRLAKNEAWERTCA | |
| | CAGCAAAACACGAAAGAGCUGAGAUCGAUCAUCGAUUAUAUUAUUAUAAGGCA | | NVNSKLGFGRAKEAWSVLKALRQDTKGKSN | |
| | GACCUCUAGCUUCAAAGCUGCUGACGUCAGAUCAUACCGUGGAGCUCAGUGCG | | LQLVTQKEWEEYFKKLLNEDRDEYLEEGTVE | |
| | GUAGCGACCACUAUCUCGUAAAAAUGAAAAGCUUUUGGCCGUGGAAAAACGCC | | ENEHHDDEILISESEVLQVLRTGKNGKSPGPG | |
| | ACCAAUGAUACAAGCAACAUCAAUAAGAUGAACUGUUCCGAGAAGGUGCAAAA | | NINMEFLKYGGDKIVKLILQLFNKMLHGDSV | |
| | CGUGCAUUUCAAUAUCGAUUCCCUGCAGGACGAGAGCAUUCGGACAUUUUUCA | | PKEMKLGYISTIFKKGDRKICSNYRGICVTNT | |
| | AGGCCCGUAUGGAGAGGACCUUGGACGAGAGUUUCGAGGGGUCCACAGAAGAG | | LMRIFGKIIKNKLEKNFRTQQEQCGFTAGRSC | |
| | AUCUACGAGUAUAUAAAGACAAAAGUCAAAAACGUUGCCUCUGAGGUGCUGGG | | VDHIFTLRQILEKHREKSKNVGLIFIDLEKAYD | |
| | CAUCAAGGAAAAUAAUCCUAAGCGAGCCGCAGAGUGGUGGUCUGAAGAGAUUG | | TVPRKLLWRALHRANINTSLIKIIEQMYKDNI | |
| | AGACAUCUGUGAAAGAGAAGCGCAAUGCAUUCGUGCAGUGGCUGAACGAUAAG | | CQVKIGNTLSQKFRTSKGLLQGCPMSPTLFKI | |
| | UCAGAGGGUACUCGAAGUAAGUACAAGGAAAAAAAGAAUGAAGUGGAAAAAA | | YIDICLRTWSQKCNSMGLEIRDGVYLHHLLF | |
| | AGAUCAGACUAGCUAAAAAUGAAGCAUGGGAACGGACCUGUGCCAAUGUGAAC | | ADDQVVIAQDGEDANYMCNQLAIAYKNWG | |
| | AGCAAGCUGGGAUUCGGACGCGCCAAGGAAGCGUGGUCUGUCCUCAAAGCUUU | | LKINYQKTEYLTNDPHELRIEGKKIKKVNTFC | |
| | ACGCCAGGACACCAAGGGUAAAAGUAACCUGCAACUUGUUACUCAGAAGGAGU | | YLGSILETEGKSDSEINKRISSGRKVIGMLNSV | |
| | GGGAAGAGUAUUUUAAGAAACUUCUGAAUGAAGAUCGCGACGAGUACCUCGAG | | LWSRNVMNRTKKIIYKSIFESAVLYGAETWTI | |
| | GAGGGCACAGUGGAAGAGAAUGAACAUCACGAUGAUGAGAUUCUGAUUAGCGA | | NQKIITKKLQALEMDFWRRSARISRKEKRRN | |
| | AAGCGAAGUACUCCAGGUCCUGAGGACGGGGAAAAACGGCAAGUCCCCUGGAC | | TEVIKRMEIIERIDEVMDRKKLRWYGHVRRM | |
| | CUGGGAAUAUUAACAUGGAGUUUCUCAAAUACGGAGGGGACAAGAUCGUAAAG | | EDTRIPKLVLEWQPEGRRRRGRPVTTWIKNV | |
| | CUCAUUCUUCAGCUGUUCAACAAGAUGCUACAUGGCGACUCUGUGCCAAAAGA | | QLTMNRLGAEEEDTQDRHTWRNIVNNGGGS | |
| | GAUGAAGUUGGGGUAUAUCUCCACAAUUUUUAAGAAAGGCGACCGAAAGAUUU | | PKKKRKVSGSETPGTSESATPESVSGWRLFKK | |
| | GUUCAAAUUACAGGGGUAUUUGUGUAACUAACACCCUAAUGCGGAUCUUCGGA | | IS | |
| | AAAAUUAUCAAGAAUAAACUGGAAAAGAACUUUCGGACGCAACAGGAACAGUG | | | |
| | CGGUUUUACAGCGGGGCGCUCUUGCGUGGAUCACAUCUUCACCCUGAGACAGA | | | |
| | UAUUGGAGAAGCACAGAGAAAAAAGUAAAAACGUGGGACUUAUUUUCAUAGAC | | | |
| | UUAGAGAAAGCAUACGACACUGUGCCCCGGAAAUUGUUAUGGCGGGCCCUACA | | | |
| | CAGGGCAAAUAUCAACACAUCACUGAUUAAAAUUAUCGAGCAGAUGUACAAAG | | | |
| | ACAACAUUUGUCAGGUGAAGAUCGGCAACACCCUGAGCCAGAAAUUUCGAACU | | | |
| | AGUAAGGGGCUGUUGCAGGGAUGCCCUAUGUCCCCAACUCUCUUUAAGAUCUA | | | |
| | UAUCGAUAUUUGUCUGCGGACCUGGUCACAAAAAUGUAAUUCCAUGGGUCUUG | | | |
| | AGAUCCGGGAUGGCGUAUAUCUCCAUCAUUUACUAUUCGCCGAUGAUCAAGUG | | | |
| | GUUAUCGCCCAGGACGGCGAGGACGCCAAUUACAUGUGUAACCAAUUAGCCAU | | | |
| | AGCUUACAAAAACUGGGGCCUGAAGAUCAAUUACCAAAAAACUGAGUAUCUGA | | | |
| | CGAACGACCCGCACGAACUGAGAAUCGAGGGGAAGAAGAUUAAAAAGGUGAAC | | | |
| | ACCUUCUGCUACUUGGGCAGUAUCCUUGAAACCGAAGGGAAGUCAGACAGUGA | | | |
| | GAUUAAUAAAAGGAUUAGCUCCGGCCGUAAGGUCAUCGGCAUGCUGAAUUCAG | | | |
| | UUCUGUGGAGCCGGAACGUCAUGAAUAGAACAAAGAAAAUUAUUUAUAAGUCU | | | |
| | AUAUUCGAGAGCGCUGUCCUCUAUGGCGCUGAGACUUGGACAAUCAACCAGAA | | | |
| | ACAUACCAAAAAGCUGCAGGCACUCGAGAUGGACUUUUGGCGACGCAGUGCAC | | | |
| | GCAUCUCUCGGAAGGAAAAAAGACGGAACACCGAAGUGAUUAAAAGGAUGGAG | | | |
| | AUCAUAGAGAGAAUUGAUGAGGUCAUGGACAGGAAGAAGCUUAGGUGGUACGG | | | |
| | ACACGUCAGGAGGAUGGAGGAUACCAGGAUCCCCAAGUUGGUGCUGGAGUGGC | | | |
| | AGCCUGAGGGACGCCGCCGAAGAGGGCGCCCAGUGACUACAUGGAUAAAAAAC | | | |
| | GUUCAACUGACCAUGAACAGACUCGGCGCCGAAGAAGAGGACACCCAGGAUAG | | | |
| | GCAUACUUGGCGGAAUAUUGUGAAUAAUGGCGGCGGCUCCCCCAAGAAGAAGC | | | |
| | GGAAGGUGAGCGGCAGCGAGACUCCCGGGACCUCAGAGUCCGCCACACCCGAA | | | |
| | AGUGUGAGCGGCUGGCGGCUGUUCAAGAAGAUUAGCUGAGCUGGAGCCUCGGU | | | |
| | GGCCAUGCUUCUUGCCCCUUGGGCCUCCCCCCAGCCCCUCCUCCCCUUCCUGCA | | | |
| | CCCGUACCCCCGUGGUCUUUGAAUAAAGUCUGAAAAAAAAAAAAAAAAAAAAA | | | |
| | AAAAAAAAAAAAAAAAAAAAAAAAAAAAAAAAAAAAAAAAAAAAAAAAAAAA | | | |
| | AAAAAAAA | | | |
| RTE- | AGGAAAUAAGAGAGAAAAGAAGAGUAAGAAGAAAUAUAAGAGCCACCAUGGAC | 15,019 | MDSTAHPNQGRGLEKVSQTLPALQTPGQHTA | 15,003 |
| 1_MD | UCCACAGCUCAUCCCAACCAGGGACGUGGCCUGGAGAAGGUGUCCCAGACUCU | | AGGSSPLSGRNQRKNTKKLLLGAWNIRTLLD | |
| RNAIVT | GCCUGCGCUGCAGACCCCAGGGCAGCACACUGCCGCUGGGGGAUCCUCUCCCCU | | RENTPRPERRTALIGKELARYNIDIAALSETRL | |
| 1123 | GUCCGGCAGGAAUCAGCGGAAGAACACCAAAAAGCUGUUGCUGGGCGCCUGGA | | PEEGSLSEPTTGYTFFWKGRASNEDRIHGVGL | |
| | ACAUCCGCACCCUGCUCGACCGCGAAAAUACUCCCCGUCCUGAAAGACGCACGG | | AIKTSLLKQLPDLPVGISERLMKIRLPLSKDRY | |
| | CACUGAUUGGGAAAGAGCUCGCCAGAUAUAACAUCGACAUCGCUGCCCUGUCC | | ATIISAYAPTLTSTEETIEQFYSDLSAVLHSVPT | |
| | GAAACCCGCCUGCCAGAGGAAGGAUCUCUGAGCGAGCCUACCACUGGCUACAC | | NDKLILLGDFNARVGQDHERWKGVLGKHGV | |
| | GUUCUUUUGGAAGGGCAGAGCAAGCAACGAGGACCGUAUCCAUGGUGUAGGCC | | GKMNNNGLLLLSKCSEFELTITNTVFRMANK | |
| | UGGCUAUCAAGACCUCUCUCCUGAAACAACUCCCAGAUCUGCCAGUCGGCAUCU | | YKTTWMHPRSKQWHLIDYIIVRRRDIQDVKIT | |
| | CAGAGAGACUGAUGAAGAUCAGGCUUCCUCUCUCAAAAGACCGCUACGCAACC | | RAMRGAECWTDIIRLVRATLQMRIAPRIIPKR | |
| | AUCAUUUCCGCAUACGCGCCGACCCUGACUAGCACCGAGGAAACCAUUGAGCA | | AQTVRAFYNVSRLRDPSYLQTFQSCLDDKLS | |
| | GUUCUACUCUGAUCUUUCAGCUGUUUUGCACUCCGUGCCUACCAACGACAAGC | | AKGPLTGSSTEKWNQFRDAVKETSKAVLGPK | |
| | UGAUCCUCCUGGGCGACUUCAACGCUCGCGUGGGUCAGGACCAUGAGCGCUGG | | QRNHQDWFDENNTAIEDLLSKKNKAFMEWQ | |
| | AAAGGCGUGCUGGGUAAGCAUGGCGUGGGUAAGAUGAACAAUAACGGCCUCCU | | NNPNSAPKKDRFKSLQATAQREIRKMQDRW | |
| | GCUCCUGUCUAAGUGUAGCGAAUUCGAGCUGACUAUUACCAACACUGUUUUUC | | WEKKAEEIQRFADMKNYKQFFSALKTVYGP | |
| | GUAUGGCUAACAAGUACAAAACUACAUGGAUGCACCCCAGGAGCAAGCAGUGG | | LKPTTTPLLSSDGDTLIKDKKGISNRWKEHFS | |
| | CACCUGAUUGACUACAUUAUCGUGCGUAGGCGGGAUAUCCAGGACGUGAAGAU | | QLLNRPSSVDQSALDQIPQNRTIEQLDVPPSIE | |
| | UACCCGUGCAAUGCGCGGCGCUGAGUGCUGGACAGACCAUCGCCUGGUGAGAG | | EVQKAIKQMSAGKAPGKDGIPTEVYKALNG | |
| | CUACUCUCCAGAUGAGGAUCGCACCGCGUCACCCUAAGCGCGCCCAGACCGUGC | | KALQAFHIVLTSIWEEEDMPPELRDASIVALY | |
| | GGGCCUUUUACAACGUAUCACGGCUUCGGGAUCCUUCCUACCUUCAGACAUUU | | KNKGSRAACDNYRGISLLSTAGKILARVILNR | |
| | CAAUCUUGCCUCGAUGACAAGCUCUCUGCUAAGGGUCCCCUCACCGGAUCUUCA | | LLSSVSEQNLPESQCGFRPDRSTIDMVFTVRQ | |
| | ACAGAGAAGUGGAACCAAUUCCGCGAUGCGGUGAAGGAAACCAGCAAAGCAGU | | MQEKCLEQNLSLYIVFIDLTKAFDTVNRDAL | |
| | CCUGGGACCGAAGCAGCGCAACCACCAAGACUGGUUUGACGAAAAUAACACUG | | WVILSKLGCPAKFVKLIQLFHVDMTGEVLSG | |
| | CCAUCGAAGACCUCCUGUCUAAAAAGAACAAAGCAUUUAUGGAGUGGCAGAAU | | GETSDRFNISNGVKQGCVLAPVLFNLFFTQVL | |
| | AACCCAAACAGUGCACCCAAGAAAGACCGUUUCAAAAGCCUGCAGGCCACCGCG | | RHAVMDLDLGVYIKYRLDGSLFDLRRLTAKT | |
| | CAGCGCGAGAUCCGGAAGAUGCAGGACCGCUGGUGGGAAAAAAAGGCCGAAGA | | KTTERLILEALFADDCALMAHQENHLQTIVD | |
| | GAUCCAGAGGUUCGCGGAUAUGAAAAACUACAAGCAGUUCUUUAGCGCACUGA | | RFSTATKLFGLTISLSKTEVLFQPAPGRPTNQP | |
| | AGACCGUGUACGGACCUCUCAAACCCACCACGACUCCCCUGCUCUCUUCCGAUG | | CITIDGTQLSNVNTFKYLGSTIANDGSLDHEIN | |
| | GUGACACUCUCAUCAAGGACAAAAAGGGAAUCAGCAACCGCUGGAAAGAACAC | | ARIQKASQALGRLRCKVLQHRGVSTATKLKV | |
| | UUUUCUCAACUUCUCAACCGCCCAAGCUCCGUGGACCAGAGCGCUCUGGACCAG | | YNAVVLSSLLYGCETWTLYRKHMKQLEQFH | |
| | AUCCCGCAGAACCGUACCAUCGAGCAGCUGGACGUGCCUCCCUCCAUCGAAGAG | | QRSLRSIMRIRWQDRITNQEVLDRANSTSIEV | |
| | GUCCAGAAGGCUAUCAAGCAGAUGUCUGCUGGGAAGGCUCCUGGCAAGGAUGG | | MVLKTQLRWSGHVIRMDPQRIPRQVFYGELS | |
| | GAUCCCAACAGAGGUCUAUAAAGCUCUCAACGGAAAGGCGCUGCAGGCCUUUC | | AGLRKQGRPKKRFKDQLKSNLKWAGITPKQ | |
| | ACAUUGUCCUGACCUCCAUCUGGGAGGAAGAGGACAUGCCACCUGAACUUAGG | | LELAASDRSSWRTHINHAATTFEDERRRRLA | |
| | GACGCCUCUAUUGUGGCCCUGUACAAGAAUAAGGGCAGCCGCGCAGCUUGUGA | | AARERRHQATTAPPVTTGVPCPMCHKLCASA | |
| | UAACUAUCGUGGCAUCUCACUCCUGAGUACAGCUGGCAAGAUUCUGGCUCGUG | | FGLQSHMRVHRRSGSETPGTSESATPESVSG | |
| | UGAUCCUGAACCGGUUGCUGAGUUCCGUUUCCGAGCAGAACCUGCCCGAAUCA | | WRLFKKIS | |
| | CAGUGCGGCUUCCGCCCCGAUCGGUCCACCAUCGAUAUGGUGUUCACCGUGCGU | | | |
| | CAGAUGCAGGAGAAGUGCCUGGAACAGAAUCUGAGCCUGUACAUCGUUUUCAU | | | |
| | CGAUCUGACUAAGGCCUUCGACACGGUGAACCGCGAUGCCCUGUGGGUGAUCC | | | |
| | UGAGCAAGCUGGGGUGCCCCGCUAAGUUCGUAAAGCUCAUCCAGUUGUUCCAC | | | |
| | GUUGAUAUGACGGGAGAGGUCCUGUCCGGUGGAGAAACCUCCGACCGGUUUAA | | | |
| | CAUCUCCAAUGGCGUGAAACAGGGCUGCGUCCUGGCCCCAGUGCUGUUCAACC | | | |
| | UGUUUUUCACACAGGUGCUGAGACAUGCGGUUAUGGAUCUUGACCUGGGUGUG | | | |
| | UAUAUCAAGUAUCGCUUGGACGGCAGUCUGUUCGAUCUCAGACGCCUGACAGC | | | |
| | UAAGACUAAGACUACCGAGCGCCUCAUUCUCGAAGCCCUGUUUGCCGACGAUU | | | |
| | GUGCCCUUAUGGCCCACCAGGAGAACCACCUGCAGACGAUUGUCGACCGCUUCU | | | |
| | CUACAGCGACCAAGCUGUUCGGCCUCACCAUCUCCCUCUCAAAGACCGAAGUCC | | | |
| | UGUUUCAGCCCGCACCCGGGAGACCCACGAAUCAGCCGUGCAUUACCAUUGACG | | | |
| | GGACCCAACUUUCCAACGUCAACACCUUCAAGUAUCUGGGAUCUACCAUCGCCA | | | |
| | AUGAUGGCAGCCUGGAUCAUGAAAUCAACGCAAGGAUCCAAAAGGCUAGCCAG | | | |
| | GCACUGGGCCGCCUCCGGUGCAAGGUCCUGCAGCACCGGGGCGUCUCCACUGCC | | | |
| | ACUAAACUGAAGGUGUACAACGCUGUCGUGCUCUCUUCCCUGCUUUAUGGCUG | | | |
| | CGAAACAUGGACUCUGUACCGGAAGCAUAUGAAACAGCUUGAGCAGUUCCACC | | | |
| | AGAGGAGCUUGCGUAGCAUCAUGAGGAUCCGCUGGCAGGACCGCAUCACCAAC | | | |
| | CAGGAGGUGCUGGACAGGGCUAAUUCCACCAGCAUCGAAGUUAUGGUGCUGAA | | | |
| | GACCCAGCUCCGUUGGUCUGGCCAUGUCAUUCGGAUGGACCCUCAGCGUAUCCC | | | |
| | CCGCCAGGUAUUCUACGGGGAACUGUCUGCGGGGCUCAGAAAGCAGGGCCGCC | | | |
| | CAAAGAAACGCUUUAAGGAUCAGCUGAAAAGCAAUCUGAAGUGGGCCGGCAUC | | | |
| | ACCCCCAAGCAGCUGGAGCUGGCGGCAUCUGACCGCUCCAGCUGGCGCACCCAC | | | |
| | AUUAACCACGCAGCUACUACCUUUGAGGACGAACGCCGGCGCAGACUGGCCGC | | | |
| | UGCACGCGAGAGGCGCCACCAAGCCACUACCGCCCCUCCCGUGACCACUGGGGU | | | |
| | CCCUUGCCCUAUGUGCCACAAGCUGUGUGCGUCUGCCUUCGGUUUGCAGUCCCA | | | |
| | CAUGCGUGUGCAUCGCCGCAGCGGCAGCGAGACUCCCGGAACCUCAGAGUCCGC | | | |
| | CACACCCGAAAGUGUGAGCGGCUGGCGGCUGUUCAAGAAGAUUAGCUGAAUGG | | | |
| | UUUAUAUUGCGGCCGCUUAAUUAAGCUGCCUUCUGCGGGGCUUGCCUUCUGGC | | | |
| | CAUGCCCUUCUUCUCUCCCUUGCACCUGUACCUCUUGGUCUUUGAAUAAAGCCU | | | |
| | GAGUAGGAAGUCUAGAAAAAAAAAAAAAAAAAAAAAAAAAAAAAAAAAAAAA | | | |
| | AAAAAAAAAAAAAAAAAAAAAAAAAAAAAAAAAAAAAAAAAAA | | | |
| Vingi- | AGGAAAUAAGAGAGAAAAGAAGAGUAAGAAGAAAUAUAAGAGCCACCAUGGAC | 15,020 | MDEYQRSLSRPLLTIMSINIEGLSLAKEELLAK | 15,002 |
| 1_Acar | GAGUACCAGCGCUCCUUGUCCCGCCCCCUGUUGACAAUCAUGAGUAUUAAUAU | | MSEDISCDILCIQETHRDITMRRPKILGMQLA | |
| RNAIVT | CGAAGGCUUGAGCCUCGCGAAAGAGGAACUGCUUGCCAAGAUGUCCGAGGACA | | VERPHRQYGSAIFVRSGVAISATSLTEVNNIEI | |
| 696 | UCAGCUGCGACAUUCUGUGCAUCCAAGAGACCCAUCGCGACAUCACCAUGCGUC | | LSVELDSCTVSSLYKPPGADFYFTPPTSCHNH | |
| | GCCCCAAGAUCCUCGGUAUGCAGCUUGCGGUGGAGCGCCCUCACCGCCAGUACG | | EAHFVVGDFNSHSCVWGYDEDDRNGEAVLT | |
| | GCUCCGCGAUUUUCGUGCGUUCCGGUGUGGCCAUUUCUGCUACCAGCCUCACCG | | WADNSRMSLLHDSKLPPSFNSGRWKRGYNP | |
| | AGGUUAACAAUAUUGAGAUCCUGUCAGUGGAGCUCGAUUCCUGUACCGUCUCC | | DLIFVKESISHQCTKRVLNPIPNTQHRPICCVA | |
| | AGUCUGUACAAACCUCCCGGUGCAGACUUCUACUUUACUCCCCCGACGUCUUGC | | YAAVRPKSVPFRRRYNFNKANWTKFTETLEA | |
| | CACAACCAUGAGGCUCAUUUUGUGGUUGGUGAUUUCAAUUCCCACUCCUGCGU | | AISDIEPSIENYDLFVEAVKRSSRLSIPRGCRTS | |
| | GUGGGGCUAUGACGAGGAUGACCGCAACGGCGAAGCUGUUCUGACCUGGGCCG | | YLPGLNEESLNQLQEYLRLFQENPYSDGTIAA | |
| | AUAACUCCCGCAUGUCCCUGCUCCACGAUAGUAAGCUGCCUCCCUCCUUCAACA | | GQKLSTALANAKKDRWIELLENLDMSKSSRK | |
| | GUGGGAGAUGGAAGCGCGGGUACAACCCUGAUCUUAUCUUCGUCAAAGAAUCC | | AWQLLRRLDSDPLVNPGHANVTPDQIAHQLI | |
| | AUCUCUCAUCAGUGUACCAAGCGCGUCCUCAACCCGAUUCCUAACACUCAGCAC | | QNGKTNCSRIKMKINRVPELETHQLSSPLNLK | |
| | CGCCCCAUUUGUUGCGUAGCAUAUGCUGCCGUGCGUCCCAAGUCUGUCCCCUUC | | ELREAIKRCKTGKAPGLDDLMMEQIKHLGAK | |
| | AGAAGGAGAUACAACUUCAACAAGGCUAACUGGACCAAGUUUACCGAAACCUU | | AENWLLKFYNQCLAHKQIPRAWRKTKIIAIL | |
| | GGAAGCGGCAAUCUCCGACAUUGAGCCAUCUAUCGAGAAUUAUGAUCUCUUCG | | KPGKDASNARNYRPISLLCHLYKVYERMLLN | |
| | UUGAGGCAGUCAAGCGGUCCUCUCGCCUGAGUAUCCCCCGUGGAUGUAGGACC | | RLGPVIEPKLIAQQAGFRPGKNCTGQILHLTE | |
| | UCCUACCUGCCCGGCCUGAACGAAGAGUCACUCAAUCAGCUGCAGGAAUACCU | | HIEEGYEKGCITGTVFVDLTAAYDTVQHRKM | |
| | GCGCCUGUUCCAAGAGAAUCCUUACUCCGAUGGCACCAUCGCUGCCGGACAAA | | LHKVYHITRDFDFTKTVQTLLENRSFYVEFQG | |
| | AACUGAGCACCGCUCUGGCGAAUGCUAAGAAAGACCGCUGGAUCGAGCUGCUC | | QKSRWRRQKNGLPQGSVLAPTLFNIFTNDQP | |
| | GAGAACCUGGACAUGUCCAAGUCUAGCCGGAAAGCUUGGCAGCUCCUGCGCCG | | QPPLTKSFIYADDLGLTTQAKDFETVEKQLTN | |
| | GCUGGACAGCGACCCCCUCGUGAAUCCUGGCCAUGCCAAUGUAACGCCGGACCA | | ALKDLSSYYKENHLKPNPAKTQVCAFHLRNR | |
| | GAUCGCCCAUCAACUGAUCCAGAACGGCAAGACCAAUUGCAGUAGAAUCAAGA | | EANRKLKVTWEGQELEHCFHPKYLGVTLDR | |
| | UGAAGAUCAACAGAGUACCCGAGUUGGAAACACACCAGCUGUCUAGUCCUCUC | | TLTYRKHCMNTKHKVAARNNILRKLTGSAW | |
| | AACCUCAAAGAGCUCCGCGAAGCAAUCAAAAGGUGUAAGACGGGCAAAGCUCC | | GADPQVIRTSALALSFSTAEYACPVWHKSAH | |
| | CGGUCUGGAUGACCUGAUGAUGGAACAGAUCAAACACUUGGGGGCCAAGGCUG | | AKQVDIALNETCRIITGCLKPTPVDKLYKLAG | |
| | AGAACUGGUUGCUGAAAUUCUACAACCAGUGCCUGGCUCACAAGCAGAUCCCC | | IAPPDVRREVAANGERKKVEHCESHPLHGYH | |
| | CGUGCGUGGCGCAAGACUAAGAUUAUCGCCAUCUUGAAGCCGGGCAAGGAUGC | | PPPTRLKSRKGFMRTTTPLDVPPAAARVSLW | |
| | UAGUAACGCCCGCAACUACCGUCCUAUUUCCCUCCUGUGUCACCUUUACAAGGU | | AAKPGNSNWMAPQEGLPPGANQEWATWKS | |
| | GUACGAGCGCAUGCUCCUGAACCGCUUGGGCCCCGUCAUCGAGCCUAAACUGA | | LNRLRSGVGRSKDNLARWHYLEESSTLCDCG | |
| | UCGCCCAACAGGCUGGUUUCCGCCCAGGUAAGAACUGCACUGGACAGAUCCUG | | AEQTTQHMYACPQCPASCTEEELFKATDNAV | |
| | CACCUUACGGAGCACAUCGAGGAAGGUUACGAGAAGGGCUGCAUCACCGGAAC | | AVARFWSKTIGGGSPKKKRKVSGSETPGTSES | |
| | CGUCUUCGUGGACCUGACUGCGGCCUACGACACGGUUCAGCACAGAAAAAUGC | | ATPESVSGWRLFKKIS | |
| | UCCACAAGGUAUAUCAUAUUACCCGGGAUUUCGACUUCACCAAAACAGUGCAG | | | |
| | ACGCUUCUGGAGAAUCGCUCCUUUUACGUCGAGUUCCAGGGUCAGAAAAGUCG | | | |
| | GUGGAGGCGCCAGAAGAACGGCUUGCCCCAGGGUAGCGUGCUCGCCCCAACCCU | | | |
| | GUUUAAUAUCUUCACCAACGACCAGCCACAGCCCCCGCUGACAAAAUCCUUUAU | | | |
| | CUAUGCCGACGAUCUUGGCCUGACCACGCAAGCCAAGGACUUUGAGACAGUCG | | | |
| | AGAAACAGCUCACAAACGCACUCAAGGACCUGAGUUCCUAUUACAAGGAGAAC | | | |
| | CAUCUCAAACCUAACCCCGCUAAGACACAGGUCUGCGCUUUUCACCUGAGGAA | | | |
| | UCGGGAGGCCAACCGUAAACUGAAGGUGACAUGGGAGGGACAGGAACUGGAGC | | | |
| | ACUGUUUCCACCCCAAGUAUUUGGGCGUGACUCUCGAUCGCACCCUUACGUAU | | | |
| | CGGAAGCACUGCAUGAACACUAAGCAUAAGGUGGCGGCCAGAAAUAACAUCCU | | | |
| | GCGUAAACUCACAGGAAGCGCCUGGGGCGCCGACCCACAGGUCAUCCGUACCAG | | | |
| | UGCUCUGGCCCUGUCUUUCUCCACUGCUGAAUAUGCGUGUCCGGUGUGGCACA | | | |
| | AGUCCGCCCACGCGAAGCAGGUGGAUAUUGCCCUGAACGAGACAUGCCGGAUU | | | |
| | AUCACUGGUUGCCUCAAGCCUACCCCCGUCGACAAGCUCUAUAAGUUGGCCGGC | | | |
| | AUUGCACCCCCUGAUGUCCGUCGCGAAGUCGCAGCCAAUGGCGAGCGGAAAAA | | | |
| | GGUAGAACACUGCGAAUCACAUCCGCUGCAUGGAUAUCAUCCCCCUCCCACCCG | | | |
| | GCUUAAGAGCCGUAAGGGCUUCAUGCGCACAACCACUCCGCUGGAUGUGCCUC | | | |
| | CCGCUGCCGCGCGCGUGAGUCUGUGGGCCGCUAAGCCCGGUAAUUCCAAUUGG | | | |
| | AUGGCACCGCAAGAGGGCCUCCCGCCCGGAGCCAAUCAAGAGUGGGCCACAUG | | | |
| | GAAGUCCUUGAACAGGCUGCGCUCCGGUGUGGGACGCUCUAAGGACAACCUGG | | | |
| | CCCGUUGGCACUAUCUGGAAGAGUCAUCCACCCUGUGCGACUGUGGUGCUGAA | | | |
| | CAGACUACCCAGCACAUGUACGCUUGCCCCCAAUGCCCUGCCUCCUGCACGGAA | | | |
| | GAGGAACUCUUUAAGGCAACCGAUAACGCAGUGGCAGUGGCGCGCUUUUGGAG | | | |
| | UAAGACAAUCGGCGGCGGCUCCCCCAAGAAGAAGCGGAAGGUGAGCGGCAGCG | | | |
| | AGACUCCCGGGACCUCAGAGUCCGCCACACCCGAAAGUGUGAGCGGCUGGCGGC | | | |
| | UGUUCAAGAAGAUUAGCUGAUUAAUUAAGCUGGAGCCUCGGUGGCCAUGCUUC | | | |
| | UUGCCCCUUGGGCCUCCCCCCAGCCCCUCCUCCCCUUCCUGCACCCGUACCCCCG | | | |
| | UGGUCUUUGAAUAAAGUCUGAUCUAGAAAAAAAAAAAAAAAAAAAAAAAAAA | | | |
| | AAAAAAAAAAAAAAAAAAAAAAAAAAAAAAAAAAAAAAAAAAAAAAAAAAAA | | | |
| | AA | | | |
| CR1- | AGGAAAUAAGAGAGAAAAGAAGAGUAAGAAGAAAUAUAAGAGCCACCAUGCUG | 15,021 | MLYTIFLVYGILCFFFVYFCIYLYITVYLCFLFF | 15517 |
| 1_PH | UAUACCAUCUUCCUCGUUUAUGGGAUCCUGUGUUUUUUCUUUGUGUAUUUCUG | | LFLLLCGDVESNPGPGRARGCRLLYCNIRGLH | |
| RNAIVT | CAUCUACCUGUACAUCACUGUUUAUCUUUGUUUUCUGUUCUUUCUGUUUCUGC | | ANLAELDFVSRGVDVVCCSETLVAGRRHDA | |
| 636 | UUCUGUGCGGUGACGUGGAAAGCAAUCCUGGACCAGGCAGAGCCCGUGGAUGU | | ELALAGFQSPFRRLCGSGPGFRGMAVYVRSG | |
| | CGGUUGCUGUAUUGCAACAUCCGCGGUCUGCAUGCUAAUCUGGCCGAGCUGGA | | FCAYRQSVHECACHEIIVVRVCGRLNNYYLF | |
| | CUUUGUUAGUCGCGGGGUGGACGUGGUCUGCUGUUCCGAGACUCUGGUGGCUG | | SLYRSPATDDSLYDCLLTAMASIQSTDPKAAF | |
| | GGCGUCGCCACGAUGCCGAGUUGGCCCUCGCUGGCUUCCAGUCCCCCUUUCGUA | | VFVGDVNAHHRDWLGSASPTDCHGVAALDF | |
| | GGCUCUGUGGCUCAGGCCCUGGCUUCCGUGGCAUGGCGGUGUACGUCAGGUCU | | CTLSSCVQLVRGSTHIAGNCLDLVMTDVPDL | |
| | GGGUUCUGUGCCUACCGGCAGAGUGUGCACGAAUGUGCCUGCCACGAGAUUAU | | MTVTVGSPIGSSDHSHLVVSLDLNQVVPVVD | |
| | CGUCGUUCGCGUGUGUGGACGUCUGAACAAUUACUAUCUGUUCUCUCUCUACC | | TRRVVFVKSRANWVAITRAVRTLPWRQIIIIS | |
| | GCUCCCCGGCCACCGAUGACUCCCUUUACGAUUGCCUUCUGACUGCCAUGGCCA | | EDPVSELNDLTVSILERFVPKRTILVRSRDKP | |
| | GCAUCCAGUCCACUGAUCCGAAGGCAGCCUUCGUCUUCGUUGGCGACGUAAAC | | WFDDQCRLAFEAKQAAYRAWRRSRDRTLW | |
| | GCACAUCACCGGGACUGGCUGGGAAGCGCCUCUCCAACAGAUUGUCACGGCGU | | QTYVDRRAEAKRVYEEAQRRLRQRSRESLLS | |
| | CGCCGCUCUGGACUUUUGCACCCUUUCCAGCUGCGUGCAACUCGUCCGUGGGUC | | IDHPHRWWSELKGSVFGAEPSLPPLVGPGGGI | |
| | CACUCAUAUCGCUGGAAACUGUCUGGAUCUGGUGAUGACCGACGUGCCUGACC | | ITDPLARAELLSAHFDGKQSRDVIALPHGCHP | |
| | UGAUGACCGUGACCGUCGGAUCCCCGAUUGGAAGUAGCGACCACUCCCACCUG | | EPRLTSLAFRSGAIKVLLEGLDPYGGVDPVG | |
| | GUGGUCUCACUGGAUCUCAACCAGGUCGUGCCUGUUGUGGAUACACGCAGGGU | | MFPLFYKQLADVLAPKLAVIFRRLIRLGNFPR | |
| | UGUGUUCGUGAAAAGCCGCGCCAACUGGGUCGCCAUCACUCGCGCUGUGAGGA | | CWRTGNITPIPKGPVSPYVANYRPITLTPILSK | |
| | CUCUGCCUUGGCGGCAGAUUAUCCACUCCGAGGACCCUGUAAGUGAGCUCAAU | | VFEKLIAGKLGRFAEVTGLLPAGQFAYRIGLG | |
| | GACCUGACCGUGUCUAUUCUGGAACGCUUUGUUCCCAAGCGCACCAUCCUCGU | | CCDALLSVSHHLQSALDCRSEARLVQLDFSA | |
| | GAGAAGCCGCGAUAAGCCUUGGUUCGAUGACCAGUGUAGACUGGCUUUCGAGG | | AFDRVNHRGLLYKLESFGVGGRVLSIIRDFVS | |
| | CUAAGCAGGCGGCUUACCGUGCUUGGCGUAGGAGUCGGGACCGCACGUUGUGG | | ERTQSVSVDGVLSASVGVVSGVPQGSVLGPL | |
| | CAGACCUAUGUGGACAGGCGGGCCGAGGCCAAGCGCGUGUAUGAAGAGGCUCA | | LFVLYTSDMFSSLENTLINYADDSTLMAVIPA | |
| | GAGGCGCCUUCGCCAGAGGAGCCGUGAAAGCCUGCUCAGUAUUGACCACCCUC | | PRLRDAVAQSLNRDLSRISAWCSAWSMKLN | |
| | AUCGCUGGUGGUCAGAGCUCAAGGGCUCCGUGUUCGGCGCCGAACCCAGCCUG | | ASKTKSMIISRSRTLVPQHPQLEIDDTLLQESS | |
| | CCCCCUCUCGUUGGUCCUGGCGGUGGAAUCAUUACCGAUCCCCUCGCCAGGGCC | | SLEILGVVFDEKLTFEPHIRRLVSRASTKIGLL | |
| | GAGCUGCUCUCCGCACAUUUUGAUGGAAAGCAGUCACGCGAUGUCAUCGCCUU | | RKVNSVFGDSQVARRCFYAFLLPVLEYCSPV | |
| | GCCCCACGGUUGCCACCCGGAGCCCCGCCUCACAUCUCUGGCCUUCAGGUCCGG | | WASAADTHLRLLDRLVSSASRLCADNDLVN | |
| | CGCUAUCAAGGUCCUCCUGGAAGGACUCGACCCCUACGGUGGCGUUGACCCCGU | | LSHRRRVAELCMFYKVYNNERHWLYSSLPA | |
| | CGGCAUGUUUCCUCUGUUUUACAAGCAGCUGGCGGACGUCCUGGCCCCAAAGU | | LKVFGRETRAACGAHSFTLEAVRCRTNQFCR | |
| | UGGCCGUUAUCUUUCGUCGGCUGAUCCGCCUCGGCAACUUUCCCCGCUGCUGGC | | CFVPWSAKVWNLLPASAFGRIGLQAFKSAVN | |
| | GCACUGGUAAUAUCACUCCCAUCCCCAAGGGCCCCGUGAGUCCUUACGUGGCCA | | GFLLDFLGGGSPKKKRKVSGSETPGTSESATP | |
| | AUUACCGUCCCAUCACCUUGACACCUAUCCUGUCUAAGGUUUUCGAAAAGCUG | | ESVSGWRLFKKIS | |
| | AUCGCAGGCAAGCUGGGUCGCUUUGCUGAAGUCACUGGCUUGCUCCCUGCUGG | | | |
| | UCAGUUUGCUUAUCGCAUCGGUCUGGGCUGCUGUGACGCUCUCCUGUCCGUGA | | | |
| | GCCAUCACCUGCAGUCCGCCCUGGACUGCCGCUCCGAAGCCCGCCUGGUGCAGC | | | |
| | UCGAUUUCUCCGCAGCGUUUGACCGCGUUAAUCACAGGGGUCUUCUGUAUAAA | | | |
| | UUGGAGUCCUUCGGCGUCGGGGGCCGGGUGCUGUCAAUUAUCCGUGAUUUCGU | | | |
| | AUCCGAGCGCACCCAGUCUGUAUCCGUGGACGGUGUUCUGUCCGCCAGCGUCG | | | |
| | GCGUGGUUAGCGGAGUCCCCCAGGGUAGCGUGCUGGGACCUCUGCUCUUUGUA | | | |
| | UUGUAUACGUCCGACAUGUUCUCUAGCCUGGAGAAUACCCUCAUUAACUAUGC | | | |
| | AGAUGACUCCACUUUGAUGGCAGUUAUUCCGGCCCCACGCCUGCGUGACGCCG | | | |
| | UAGCGCAGUCCCUGAAUAGGGACCUGUCACGCAUCAGUGCCUGGUGUUCAGCU | | | |
| | UGGUCCAUGAAGCUGAACGCGUCCAAGACCAAAAGCAUGAUUAUCUCAAGGUC | | | |
| | CAGGACUUUGGUGCCUCAACACCCCCAGCUGGAGAUCGACGAUACUUUGCUGC | | | |
| | AGGAGUCCUCAUCCCUGGAAAUCCUGGGCGUCGUGUUCGAUGAGAAGCUCACC | | | |
| | UUCGAGCCUCACAUCAGGCGGCUGGUGAGCCGCGCGUCUACCAAGAUCGGGCU | | | |
| | GCUCCGCAAGGUGAACUCCGUGUUCGGCGAUUCCCAGGUCGCGAGGCGUUGCU | | | |
| | UUUACGCUUUCCUGCUCCCUGUGCUGGAGUACUGCUCUCCGGUAUGGGCGAGU | | | |
| | GCCGCGGAUACCCAUCUUCGUCUUCUGGACCGCCUUGUCUCCAGCGCCAGCCGU | | | |
| | UUGUGUGCUGACAACGAUCUGGUGAACCUGAGUCACCGUCGGCGCGUGGCUGA | | | |
| | GCUGUGUAUGUUCUACAAGGUGUACAAUAACGAGAGGCAUUGGCUGUACUCAU | | | |
| | CCCUGCCAGCCCUGAAGGUCUUCGGGCGCGAAACGCGUGCCGCGUGCGGUGCUC | | | |
| | ACUCUUUUACGUUGGAGGCGGUUCGCUGUCGCACCAACCAGUUCUGCCGCUGC | | | |
| | UUUGUGCCUUGGUCUGCGAAGGUGUGGAACCUGCUCCCUGCCUCCGCUUUCGG | | | |
| | GAGAAUCGGUCUGCAGGCAUUUAAAAGCGCUGUGAACGGUUUCCUGUUGGACU | | | |
| | UUCUGGGCGGCGGCUCCCCCAAGAAGAAGCGGAAGGUGAGCGGCAGCGAGACU | | | |
| | CCCGGGACCUCAGAGUCCGCCACACCCGAAAGUGUGAGCGGCUGGCGGCUGUUC | | | |
| | AAGAAGAUUAGCUGAUUAAUUAAGCUGCCUUCUGCGGGGCUUGCCUUCUGGCC | | | |
| | AUGCCCUUCUUCUCUCCCUUGCACCUGUACCUCUUGGUCUUUGAAUAAAGCCU | | | |
| | GAGUAGGAAGUCUAGAAAAAAAAAAAAAAAAAAAAAAAAAAAAAAAAAAAAA | | | |
| | AAAAAAAAAAAAAAAAAAAAAAAAAAAAAAAAAAAAAAAAAAA | | | |
| RTE- | AGGAAAUAAGAGAGAAAAGAAGAGUAAGAAGAAAUAUAAGAGCCACCAUGAGC | 15,022 | MSGTPRVAFDSGKDLRNPIGQSPPALSRAAPG | 15518 |
| 3_BF | GGCACACCAAGAGUGGCAUUUGACAGCGGAAAGGACCUCAGAAAUCCCAUAGG | | QLGTDPSRSACFIGCLELRVLLDKWIVCRAPD | |
| RNAIVT | GCAGUCCCCACCUGCCCUCAGCCGGGCUGCCCCCGGACAGUUGGGGACAGACCC | | KQESKEKRRQKTQPIRIGSWNVRTMRTGLSD | |
| 635 | CUCACGUUCCGCAUGUUUUAUAGGGUGCCUGGAGCUUCGAGUCCUGUUGGACA | | DLTVIEDIRKTAAIDRELYRLNIDIVALQETRL | |
| | AAUGGAUCGUGUGCCGAGCUCCUGAUAAGCAGGAAUCCAAGGAGAAACGCCGC | | PDSGSLKEDSYTFFWQGKGMEETREHGVGFA | |
| | CAGAAAACUCAGCCUAUACGGAUUGGAAGCUGGAACGUCAGAACAAUGCGGAC | | VRNTLLHMIEPPTGGTERIITLRLSTHEGPVNL | |
| | AGGCCUGUCUGAUGACUUGACAGUUAUCGAGGACAUCCGUAAAACAGCCGCAA | | LCVYAPTLQATSEVKDQFYGQLDSAIKKIPVS | |
| | UUGACCGCGAACUUUAUCGCCUGAAUAUUGACAUUGUUGCGCUGCAAGAAACG | | EHIFILGDFNARVGTDQESWQTVLGHHGIGK | |
| | CGUUUGCCCGACAGCGGUUCUCUCAAGGAAGACAGCUAUACUUUCUUCUGGCA | | MNENGQRLLELCCYHNLCVTNTFFQNKAIHK | |
| | GGGUAAGGGUAUGGAGGAAACAAGGGAGCACGGCGUGGGGUUUGCCGUGAGAA | | ASWRHPRSQRWHQLDLVITRRTSLNSVCNTR | |
| | ACACUUUGCUCCACAUGAUUGAGCCUCCCACGGGUGGUACCGAGCGCAUCAUU | | AYHSADCDTDHSLIAARIKLRPKKLHHMKKK | |
| | ACACUUCGUUUAAGUACCCAUGAGGGCCCUGUGAAUCUCCUCUGUGUUUACGC | | GQPKIDVSKTMLPDRNQKFLECLEGTLNNIQP | |
| | UCCCACCCUGCAGGCGACAUCAGAGGUCAAAGAUCAAUUUUACGGGCAGUUAG | | QDAEHRWETLSKTIYSAAAQSYGKKERKNT | |
| | ACUCCGCUAUCAAGAAGAUACCCGUCAGCGAGCACAUUUUUAUCUUGGGAGAC | | DWFEAYISELEPVMDTKRKALVSYKQNPSSQ | |
| | UUUAACGCCCGGGUCGGGACGGACCAGGAGUCAUGGCAGACCGUGCUCGGACA | | NLQALKAARQEAQRASRRCANNYWLLLSERI | |
| | UCACGGGAUUGGGAAAAUGAACGAAAACGGGCAGCGGCUGCUCGAACUAUGUU | | QLASATGDIRRMYEGIKQATGKPIKKSAPLKA | |
| | GCUAUCACAACCUGUGCGUCACCAAUACCUUCUUCCAGAAUAAAGCCAUUCAU | | KSGEIITDKDKQMARWVEHYLDIYSTENSVS | |
| | AAAGCUAGUUGGAGACAUCCCAGAAGUCAACGAUGGCACCAGCUAGACUUGGU | | QDALDNIEDFSVLAELDADPTIEELSKAIDSM | |
| | AAUCACCAGGCGGACAAGCCUCAAUUCCGUUUGCAAUACCAGGGCCUAUCAUU | | SNGKAPGEDNIPAEIIKSGKSVLLEPLIIELLRL | |
| | CCGCGGACUGUGAUACUGAUCACAGCCUGAUCGCUGCGAGAAUCAAGCUGAGG | | CWKEGKVPQSMRNSKIVTLYKNKGDRTDCN | |
| | CCUAAGAAACUGCACCAUAUGAAGAAGAAAGGCCAGCCAAAGAUCGAUGUGUC | | SYRGISLLSIVGKVFAKVVLTRLQVLADRVYP | |
| | UAAAACUAUGCUCCCCGACAGGAACCAGAAGUUCCUUGAAUGCCUGGAAGGCA | | ESQCGFRAERSTTDMIFSVRQLQEKCREQQRP | |
| | CACUCAACAACAUCCAACCGCAGGAUGCAGAGCACAGGUGGGAGACCCUGUCC | | LYIAFIDLTKAFDLVSRRGLFQLLRKIGCPPQL | |
| | AAGACCAUCUACAGCGCCGCCGCACAGUCCUAUGGCAAGAAGGAGCGGAAGAA | | LDIIISFHEDMKGVVSFDGETSEPFAIRSGVKQ | |
| | CACAGACUGGUUCGAGGCUUAUAUCUCUGAGCUUGAGCCUGUGAUGGAUACGA | | GCVLAPTLFGIFFSLLLKSAFGHSTQGVHLHT | |
| | AGAGGAAAGCCCUGGUGAGUUAUAAGCAGAAUCCCAGCUCACAAAAUCUGCAG | | RSDGKLFNLARLRAKTKVRSVLIRDMLFADD | |
| | GCCCUGAAGGCUGCCCGCCAGGAAGCUCAGAGAGCCUCACGACGAUGCGCCAAC | | AALVAHVEDELQQLLNQFAHACSEFALTISIK | |
| | AACUACUGGUUGCUACUGUCUGAGAGAAUUCAACUAGCGUCCGCGACCGGCGA | | KTVVMGQDVPQPPVVTIGSEVLEVTDHFTYL | |
| | UAUUCGCCGCAUGUAUGAGGGCAUCAAGCAAGCCACCGGUAAACCUAUCAAAA | | GSTVTSNLSLDKEIDRRIARAAGVMTKLGTR | |
| | AAUCUGCCCCCCUUAAGGCUAAAUCCGGCGAGAUUAUAACCGAUAAGGACAAG | | VWNNSIILTLNTKLEVYRSCVLSTLLYGSETW | |
| | CAAAUGGCUCGCUGGGUCGAACACUACCUGGAUAUUUAUUCUACAGAGAACUC | | TTYAKQENRLESFHLRCLRRILGISWRDRVPN | |
| | UGUCUCUCAGGAUGCCCUGGAUAACAUCGAGGAUUUUUCUGUGCUGGCCGAAC | | TTVLERSCSLSIHLLLCQRRLRWLGHVSRMK | |
| | UCGAUGCUGAUCCAACCAUAGAAGAGUUAUCUAAGGCUAUAGACUCAAUGUCA | | DGRIPKDILFGELATGKRPVGRPALRFRDVCK | |
| | AACGGCAAAGCACCAGGCGAAGACAAUAUUCCAGCCGAAAUUAUAAAAAGUGG | | RDLKLTDIDPASWEQIAADRNRWRHTVKDG | |
| | UAAAUCUGUGCUCCUUGAACCGCUGCAUGAAUUACUGAGACUGUGUUGGAAAG | | LAKGQERRTEHLESRRRKRKEKPQQGNPSAFI | |
| | AGGGGAAAGUCCCGCAGUCAAUGCGAAACUCGAAAAUCGUAACUCUGUACAAG | | CPNCGRDCHARIGLQSHSRRCQPPSGSETPGT | |
| | AAUAAAGGAGAUAGGACUGACUGCAAUUCCUACCGCGGCAUAAGCUUGUUGUC | | SESATPESVSGWRLFKKIS | |
| | UAUCGUGGGCAAAGUAUUCGCUAAAGUGGUGCUAACCAGGCUGCAGGUGCUGG | | | |
| | CUGAUCGCGUGUACCCAGAGAGCCAGUGUGGCUUCCGAGCCGAGAGGAGUACC | | | |
| | ACUGACAUGAUCUUUUCAGUGCGGCAGCUUCAGGAGAAGUGCCGCGAGCAACA | | | |
| | ACGGCCUCUGUACAUCGCCUUUAUUGACUUGACUAAGGCAUUCGACUUAGUGA | | | |
| | GUAGGAGGGGACUUUUCCAGCUCCUGAGGAAGAUUGGUUGUCCUCCACAACUG | | | |
| | CUGGAUAUAAUUAUCUCGUUCCAUGAGGAUAUGAAGGGAGUCGUUAGUUUCGA | | | |
| | UGGGGAAACAAGCGAACCAUUCGCAAUUAGGUCUGGCGUCAAGCAAGGCUGCG | | | |
| | UUCUGGCACCUACGUUGUUUGGCAUCUUUUUCUCCCUCCUCCUGAAGUCCGCA | | | |
| | UUUGGGCACAGCACUCAGGGUGUGCACCUGCAUACCCGGUCUGAUGGGAAGCU | | | |
| | UUUUAACCUCGCCCGCCUCAGGGCCAAGACUAAGGUACGCUCUGUAUUAAUCA | | | |
| | GAGAUAUGCUGUUCGCAGACGACGCAGCGCUCGUUGCUCAUGUUGAGGACGAG | | | |
| | CUUCAACAGUUAUUGAAUCAGUUCGCACAUGCAUGCUCAGAAUUCGCCCUCAC | | | |
| | AAUUAGUAUCAAAAAAACUGUCGUGAUGGGUCAGGAUGUGCCACAGCCCCCAG | | | |
| | UGGUGACGAUCGGAAGUGAGGUCCUGGAGGUUACCGACCACUUUACGUACCUU | | | |
| | GGAAGCACUGUGACCUCCAACCUGUCCCUAGACAAAGAAAUCGACAGGCGCAU | | | |
| | UGCCCGGGCAGCCGGAGUCAUGACAAAACUGGGAACCCGAGUGUGGAAUAAUA | | | |
| | GUCACCUCACGCUGAAUACUAAGCUUGAAGUGUAUAGAUCGUGUGUUCUGAGC | | | |
| | ACACUGCUUUACGGGUCCGAAACCUGGACCACCUACGCUAAGCAGGAGAAUAG | | | |
| | ACUGGAGAGCUUUCACCUUAGGUGUCUUCGUCGAAUCCUAGGCAUUUCCUGGA | | | |
| | GAGAUCGGGUUCCCAACACAACCGUGUUGGAGAGAAGCUGCUCACUGAGUAUU | | | |
| | CACCUCUUACUGUGCCAGCGGCGGCUGCGGUGGCUGGGCCACGUAAGCAGGAU | | | |
| | GAAGGACGGACGUAUCCCGAAAGAUAUUCUCUUCGGGGAACUGGCUACUGGAA | | | |
| | AGCGUCCCGUCGGCCGCCCAGCUCUCCGCUUCAGGGACGUAUGUAAAAGAGAU | | | |
| | UUGAAACUAACUGAUAUCGAUCCGGCCUCCUGGGAACAGAUCGCAGCCGACCG | | | |
| | AAACCGUUGGCGCCACACUGUGAAGGACGGACUGGCAAAAGGGCAAGAAAGAC | | | |
| | GGACAGAGCACUUAGAAUCACGGCGGCGAAAGAGAAAGGAGAAGCCUCAGCAG | | | |
| | GGUAACCCAAGUGCCUUUAUCUGUCCUAAUUGCGGCAGGGACUGCCAUGCGCG | | | |
| | GAUAGGACUGCAAUCGCAUUCAAGAAGAUGUCAGCCCCCUAGCGGCAGCGAGA | | | |
| | CUCCCGGAACCUCAGAGUCCGCCACACCCGAAAGUGUGAGCGGCUGGCGGCUGU | | | |
| | UCAAGAAGAUUAGCUGAGCUGGAGCCUCGGUGGCCAUGCUUCUUGCCCCUUGG | | | |
| | GCCUCCCCCCAGCCCCUCCUCCCCUUCCUGCACCCGUACCCCCGUGGUCUUUGA | | | |
| | AUAAAGUCUGAAAAAAAAAAAAAAAAAAAAAAAAAAAAAAAAAAAAAAAAAA | | | |
| | AAAAAAAAAAAAAAAAAAAAAAAAAAAAAAAAAAAAAAA | | | |
| RTE- | AGGAAAUAAGAGAGAAAAGAAGAGUAAGAAGAAAUAUAAGAGCCACCAUGCCC | 15,023 | MPRKNWNCGRRTGDEKRKMMFGCWNVQGI | 15,024 |
| 25_LMi | CGCAAAAAUUGGAACUGCGGGCGACGAACCGGGGAUGAAAAACGUAAGAUGAU | | STKIDLLPAELDMFNIDVVVLSETKRKGKGEE | |
| RNAIVT | GUUUGGAUGCUGGAAUGUGCAGGGAAUCUCGACAAAGAUAGAUCUGCUGCCAG | | ELDNYVHIWSGVSKAVRAKAGVSIMIQKKW | |
| 927 | CAGAACUGGACAUGUUCAACAUUGAUGUCGUGGUGCUCUCCGAGACAAAGAGG | | KKRITNWTFINERIITVEMTLFAREVVIIGVYA | |
| | AAGGGCAAGGGUGAAGAGGAGUUGGAUAAUUACGUUCACAUCUGGUCAGGGGU | | PTNDTKDKEKDAFWDTLRETIEKIPRRKELII | |
| | CUCUAAGGCUGUCAGAGCCAAGGCUGGUGUCUCCAUCAUGAUACAGAAAAAGU | | MGDMNGRVGIRESCKIVGKHGEAEYNDNGE | |
| | GGAAGAAACGUAUCACUAACUGGACGUUCAUAAAUGAGCGCAUCAUAACCGUU | | RLIDICAQFDLKITNTFFKHKDIHKYTWQQNT | |
| | GAAAUGACACUGUUUGCGCGGGAAGUUGUUAUCAUCGGCGUAUACGCCCCCAC | | KELRSIIDYIIIRQTSSFKAADVRSYRGAQCGS | |
| | CAACGACACUAAGGAUAAAGAGAAAGACGCGUUUUGGGACACUCUCAGAGAAA | | DIIYLVKMKSFWPWKNATNDTSNINKMNCSE | |
| | CCAUCGAAAAAAUCCCCAGAAGGAAGGAACUUAUCAUAAUGGGUGAUAUGAAU | | KVQNVHFNIDSLQDESIRTFFKARMERTLDES | |
| | GGCCGGGUGGGGAUUAGAGAAUCCUGCAAGAUAGUGGGCAAGCAUGGAGAAGC | | FEGSTEEIYEYIKTKVKNVASEVLGIKENNPK | |
| | AGAGUAUAACGACAAUGGAGAGCGUCUUAUUGAUAUUUGCGCCCAGUUCGACU | | RAAEWWSEEIETSVKEKRNAFVQWLNDKSE | |
| | UGAAGAUCACUAACACGUUUUUCAAGCACAAGGACAUCCACAAGUAUACAUGG | | GTRSKYKEKKNEVEKKIRLAKNEAWERTCA | |
| | CAGCAAAACACGAAAGAGCUGAGAUCGAUCAUCGAUUAUAUUAUUAUAAGGCA | | NVNSKLGFGRAKEAWSVLKALRQDTKGKSN | |
| | GACCUCUAGCUUCAAAGCUGCUGACGUCAGAUCAUACCGUGGAGCUCAGUGCG | | LQLVTQKEWEEYFKKLLNEDRDEYLEEGTVE | |
| | GUAGCGACCACUAUCUCGUAAAAAUGAAAAGCUUUUGGCCGUGGAAAAACGCC | | ENEHHDDEILISESEVLQVLRTGKNGKSPGPG | |
| | ACCAAUGAUACAAGCAACAUCAAUAAGAUGAACUGUUCCGAGAAGGUGCAAAA | | NINMEFLKYGGDKIVKLILQLFNKMLHGDSV | |
| | CGUGCAUUUCAAUAUCGAUUCCCUGCAGGACGAGAGCAUUCGGACAUUUUUCA | | PKEMKLGYISTIFKKGDRKICSNYRGICVTNT | |
| | AGGCCCGUAUGGAGAGGACCUUGGACGAGAGUUUCGAGGGGUCCACAGAAGAG | | LMRIFGKIIKNKLEKNFRTQQEQCGFTAGRSC | |
| | AUCUACGAGUAUAUAAAGACAAAAGUCAAAAACGUUGCCUCUGAGGUGCUGGG | | VDHIFTLRQILEKHREKSKNVGLIFIDLEKAYD | |
| | CAUCAAGGAAAAUAAUCCUAAGCGAGCCGCAGAGUGGUGGUCUGAAGAGAUUG | | TVPRKLLWRALHRANINTSLIKIIEQMYKDNI | |
| | AGACAUCUGUGAAAGAGAAGCGCAAUGCAUUCGUGCAGUGGCUGAACGAUAAG | | CQVKIGNTLSQKFRTSKGLLQGCPMSPTLFKI | |
| | UCAGAGGGUACUCGAAGUAAGUACAAGGAAAAAAAGAAUGAAGUGGAAAAAA | | YIDICLRTWSQKCNSMGLEIRDGVYLHHLLF | |
| | AGAUCAGACUAGCUAAAAAUGAAGCAUGGGAACGGACCUGUGCCAAUGUGAAC | | ADDQVVIAQDGEDANYMCNQLAIAYKNWG | |
| | AGCAAGCUGGGAUUCGGACGCGCCAAGGAAGCGUGGUCUGUCCUCAAAGCUUU | | LKINYQKTEYLTNDPHELRIEGKKIKKVNTFC | |
| | ACGCCAGGACACCAAGGGUAAAAGUAACCUGCAACUUGUUACUCAGAAGGAGU | | YLGSILETEGKSDSEINKRISSGRKVIGMLNSV | |
| | GGGAAGAGUAUUUUAAGAAACUUCUGAAUGAAGAUCGCGACGAGUACCUCGAG | | LWSRNVMNRTKKIIYKSIFESAVLYGAETWTI | |
| | GAGGGCACAGUGGAAGAGAAUGAACAUCACGAUGAUGAGAUUCUGAUUAGCGA | | NQKHTKKLQALEMDFWRRSARISRKEKRRN | |
| | AAGCGAAGUACUCCAGGUCCUGAGGACGGGGAAAAACGGCAAGUCCCCUGGAC | | TEVIKRMEIIERIDEVMDRKKLRWYGHVRRM | |
| | CUGGGAAUAUUAACAUGGAGUUUCUCAAAUACGGAGGGGACAAGAUCGUAAAG | | EDTRIPKLVLEWQPEGRRRRGRPVTTWIKNV | |
| | CUCAUUCUUCAGCUGUUCAACAAGAUGCUACAUGGCGACUCUGUGCCAAAAGA | | QLTMNRLGAEEEDTQDRHTWRNIVNNSGSET | |
| | GAUGAAGUUGGGGUAUAUCUCCACAAUUUUUAAGAAAGGCGACCGAAAGAUUU | | PGTSESATPESVSGWRLFKKIS | |
| | GUUCAAAUUACAGGGGUAUUUGUGUAACUAACACCCUAAUGCGGAUCUUCGGA | | | |
| | AAAAUUAUCAAGAAUAAACUGGAAAAGAACUUUCGGACGCAACAGGAACAGUG | | | |
| | CGGUUUUACAGCGGGGCGCUCUUGCGUGGAUCACAUCUUCACCCUGAGACAGA | | | |
| | UAUUGGAGAAGCACAGAGAAAAAAGUAAAAACGUGGGACUUAUUUUCAUAGAC | | | |
| | UUAGAGAAAGCAUACGACACUGUGCCCCGGAAAUUGUUAUGGCGGGCCCUACA | | | |
| | CAGGGCAAAUAUCAACACAUCACUGAUUAAAAUUAUCGAGCAGAUGUACAAAG | | | |
| | ACAACAUUUGUCAGGUGAAGAUCGGCAACACCCUGAGCCAGAAAUUUCGAACU | | | |
| | AGUAAGGGGCUGUUGCAGGGAUGCCCUAUGUCCCCAACUCUCUUUAAGAUCUA | | | |
| | UAUCGAUAUUUGUCUGCGGACCUGGUCACAAAAAUGUAAUUCCAUGGGUCUUG | | | |
| | AGAUCCGGGAUGGCGUAUAUCUCCAUCAUUUACUAUUCGCCGAUGAUCAAGUG | | | |
| | GUUAUCGCCCAGGACGGCGAGGACGCCAAUUACAUGUGUAACCAAUUAGCCAU | | | |
| | AGCUUACAAAAACUGGGGCCUGAAGAUCAAUUACCAAAAAACUGAGUAUCUGA | | | |
| | CGAACGACCCGCACGAACUGAGAAUCGAGGGGAAGAAGAUUAAAAAGGUGAAC | | | |
| | ACCUUCUGCUACUUGGGCAGUAUCCUUGAAACCGAAGGGAAGUCAGACAGUGA | | | |
| | GAUUAAUAAAAGGAUUAGCUCCGGCCGUAAGGUCAUCGGCAUGCUGAAUUCAG | | | |
| | UUCUGUGGAGCCGGAACGUCAUGAAUAGAACAAAGAAAAUUAUUUAUAAGUCU | | | |
| | AUAUUCGAGAGCGCUGUCCUCUAUGGCGCUGAGACUUGGACAAUCAACCAGAA | | | |
| | ACAUACCAAAAAGCUGCAGGCACUCGAGAUGGACUUUUGGCGACGCAGUGCAC | | | |
| | GCAUCUCUCGGAAGGAAAAAAGACGGAACACCGAAGUGAUUAAAAGGAUGGAG | | | |
| | AUCAUAGAGAGAAUUGAUGAGGUCAUGGACAGGAAGAAGCUUAGGUGGUACGG | | | |
| | ACACGUCAGGAGGAUGGAGGAUACCAGGAUCCCCAAGUUGGUGCUGGAGUGGC | | | |
| | AGCCUGAGGGACGCCGCCGAAGAGGGCGCCCAGUGACUACAUGGAUAAAAAAC | | | |
| | GUUCAACUGACCAUGAACAGACUCGGCGCCGAAGAAGAGGACACCCAGGAUAG | | | |
| | GCAUACUUGGCGGAAUAUUGUGAAUAAUAGCGGCAGCGAGACUCCCGGAACCU | | | |
| | CAGAGUCCGCCACACCCGAAAGUGUGAGCGGCUGGCGGCUGUUCAAGAAGAUU | | | |
| | AGCUGAGCUGGAGCCUCGGUGGCCAUGCUUCUUGCCCCUUGGGCCUCCCCCCAG | | | |
| | CCCCUCCUCCCCUUCCUGCACCCGUACCCCCGUGGUCUUUGAAUAAAGUCUGAA | | | |

### Example 13: Evaluation of Gene Editing of Primary Human T Cells with a BCMA CAR by a Retrotransposon-Based Gene Modifying System

The RTE1_MD gene modifying system comprising a BCMA-CAR template RNA was introduced into primary human CD3+ T cells. The BCMA CAR template RNA for use with the gene modifying polypeptide comprised the following sequences from 5′ to 3′: 5′ UTR, tkPa, BCMA CAR, Kozak, MND promoter, 3′ UTR. The BCMA CAR sequence in the template RNA comprised SEQ ID NO: 15491, encoding the amino acid of SEQ ID NO: 15490. Briefly, human CD3+ T cells stored in liquid nitrogen from donors were thawed in TexMACS culture media (Miltenyi) supplemented with 5% Human AB serum, IL2 (20 IU/mL; Miltenyi), IL7 (10 ng/mL; Miltenyi), and IL15 (5 ng/mL; Miltenyi). Cells were activated immediately post thaw by supplementing the culture media with TransACT (according to manufacturer's protocol) for two days.

Activated T cells were electroporated with mRNA encoding RTE1 MD gene modifying polypeptide (with SV40 NLS at the N-terminus of the polypeptide, SV40 NLS at the C-terminus of the polypeptide, or No NLS) and BCMA CAR template RNA 48 hours post activation. As controls, T cells were electroporated only with either the RTE1_MD driver mRNA or the BCMA CAR template alone under the same conditions. For the electroporation, 20-100 million T cells per condition were electroporated with 500 μg/mL of RNA (mass ratio of 200 template:1 driver) in OptiMEM to maintain a cell concentration of 4×10⁸ cells/mL. Post Electroporation, conditions were quenched in expansion media (TexMACS, 100 IU/mL IL2, 10 ng/mL of IL7, 5 ng/mL of IL15, 5% Human AB serum) and expanded until day 9 post initial thaw. Test and control condition cells were analyzed by flow cytometry for CAR expression on days 5, 7, and 9 post-thaw.

On day 9, CART cells were harvested, and function was assessed via a BCMA target cell killing assay. Briefly, BCMA-CART cells were co-cultured for 24 hours with BCMA-positive tumor cell lines (H929) at serially decreasing T cell (effector) to tumor cell ratios. Cells from co-culture were stained with antibodies to uniquely identify surface markers for both target cells and effector cells. Stained co-cultures were acquired on a flow cytometer with dead cell exclusion via a vital dye. Targets were positively identified during analysis and absolute counts of target cells for each condition were generated by volumetric readout by the flow cytometer. All conditions were normalized to a target only well to control for tumor growth via the following equation: % Killing=1−(number of target cells in test condition/number of target cells in normalization control)*100.

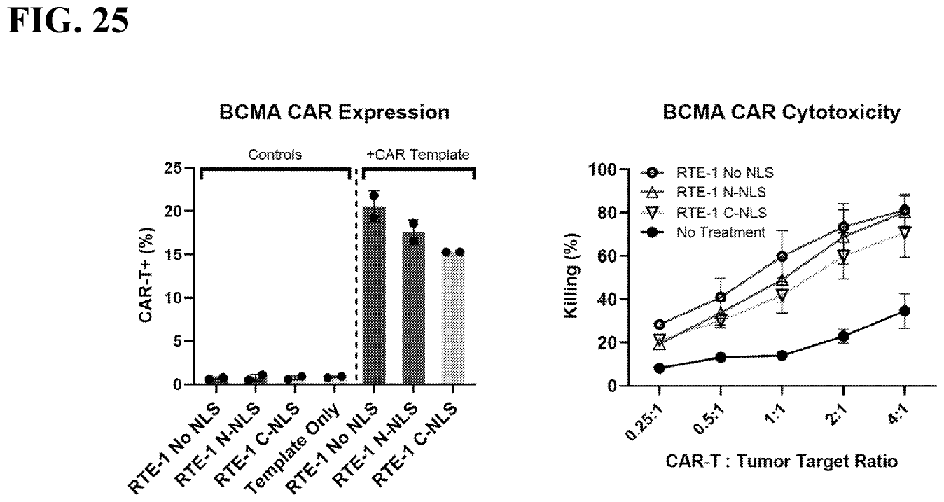

As shown in FIG. 25, the RTE-1_MD gene modifying system introduced BCMA CAR in human primary T cells obtained from two different donors. On average, ˜20% of activated T cells co-electroporated with both mRNA encoding the RTE1_MD gene modifying polypeptide and the BCMA CAR template RNA expressed the BCMA CAR at 5 days post electroporation. In comparison, cells treated with either mRNA encoding RTE1_MD gene modifying polypeptide alone or BCMA CAR template alone did not show any BCMA CAR expression. These results demonstrate that RTE1-MD gene modifying systems can be used to insert and express a therapeutic transgene (a BCMA CAR) into human primary T cells.

As shown in FIG. 25, the primary human T cells from two donors that express BCMA-CAR (as delivered by the RTE1_MD gene modifying system) killed BCMA positive tumor cells in a dose dependent manner. Overall, over 50% killing was observed for both donors with a 2 or 4 ratio of effector T cells to tumor cells. These data, taken together, indicate that the BCMA CAR T cells produced using RTE1_MD gene modifying systems represent a functional and potent CART product that controlled tumor growth in vitro.

### Example 14: Evaluation of Gene Editing of Primary Human T Cells with a CD20 CAR by a Retrotransposon-Based Gene Modifying System

The RTE1_MD gene modifying system comprising a CD20 CAR template RNA was introduced into primary human CD3+ T cells. Briefly, human CD3+ T cells stored in liquid nitrogen from one donor were thawed in TexMACS culture media (Miltenyi) supplemented with 5% Human AB serum, IL2 (20 IU/mL; Miltenyi), IL7 (10 ng/mL; Miltenyi), and IL15 (5 ng/mL; Miltenyi). Cells were activated immediately post thaw by supplementing the culture media with TransACT (according to manufacturer's protocol) for two days.

Activated T cells were electroporated with mRNA encoding RTE-1_MD gene modifying polypeptide (described in Example 6) and CD20 CAR template RNA 48 hours post activation. The CD20 CAR template RNA for use with the gene modifying polypeptide comprised the following sequences from 5′ to 3′. 5′UTR, TkPA, CD20 CAR, Kozak, MND promoter, 3′UTR. Sequences of the CD20 CAR are shown below. As a control, T cells were electroporated without RNA. For the electroporation, 0.5 million T cells per condition were electroporated with 250 ug/mL of RNA (mass ratio of 10 template:1 driver) in P3 solution with Supplement 1 to maintain a cell concentration of 25×10⁶ cells/mL. Post Electroporation, conditions were quenched in expansion media (TexMACS, 100 IU/mL IL2, 10 ng/mL of IL7, 5 ng/mL of IL15, 5% Human AB serum) and expanded until day 9 post initial thaw and activation. Test and control condition cells were analyzed by flow cytometry for CAR expression on days 5, 7 and 9 post-thaw.

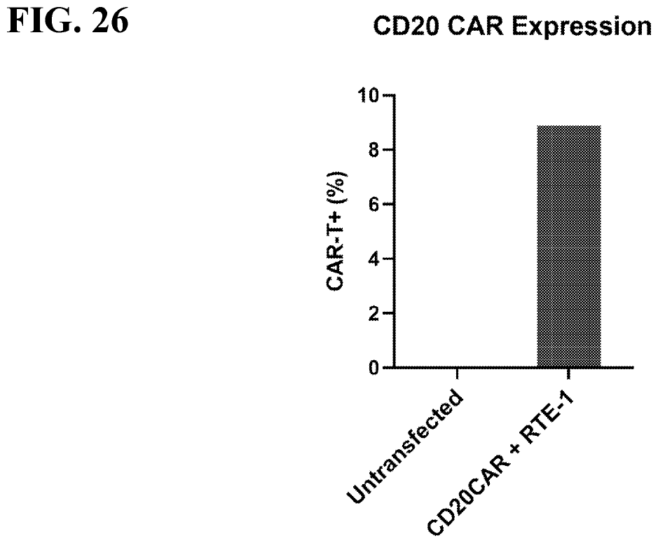

As shown in FIG. 26, the RTE-1_MD gene modifying system introduced the CD20 CAR into human primary T cells obtained from one healthy human donor. About 9% of the electroporated T cells expressed CAR at 5 days post electroporation.

CD20CAR full sequence (nucleotide):
(SEQ ID NO: 15549)
ATGGAGACAGACACACTCCTGCTATGGGTGCTGCTGCTCTGGGTTCCAGGTTCCACA
GGTGACATTGTGCTGACCCAATCTCCAGCTATCCTGTCTGCATCTCCAGGGGAGAAG
GTCACAATGACTTGCAGGGCCAGCTCAAGTGTAAATTACATGGACTGGTACCAGAA
GAAGCCAGGATCCTCCCCCAAACCCTGGATTTATGCCACATCCAACCTGGCTTCTGG
AGTCCCTGCTCGCTTCAGTGGCAGTGGGTCTGGGACCTCTTACTCTCTCACAATCAG
CAGAGTGGAGGCTGAAGATGCTGCCACTTATTACTGCCAGCAGTGGAGTTTTAATCC
ACCCACGTTCGGAGGGGGGACCAAGCTGGAAATAAAAGGCAGTACTAGCGGTGGTG
GCTCCGGGGGCGGTTCCGGTGGGGGCGGCAGCAGCGAGGTGCAGCTGCAGCAGTCT
GGGGCTGAGCTGGTGAAGCCTGGGGCCTCAGTGAAGATGTCCTGCAAGGCTTCTGG
CTACACATTTACCAGTTACAATATGCACTGGGTAAAGCAGACACCTGGACAGGGCCT
GGAATGGATTGGAGCTATTTATCCAGGAAATGGTGATACTTCCTACAATCAGAAGTT
CAAAGGCAAGGCCACATTGACTGCAGACAAATCCTCCAGCACAGCCTACATGCAGC
TCAGCAGCCTGACATCTGAGGACTCTGCGGACTATTACTGTGCAAGATCTAATTATT
ACGGTAGTAGCTACTGGTTCTTCGATGTCTGGGGCGCAGGGACCACGGTCACCGTCT
CCTCAGAGAGCAAGTACGGACCGCCCTGCCCCCCTTGCCCTATGTTCTGGGTGCTGG
TGGTGGTCGGAGGCGTGCTGGCCTGCTACAGCCTGCTGGTCACCGTGGCCTTCATCA
TCTTTTGGGTGaggagtaagaggagcaggctcctgcacagtgactacatgaacatga
ctccccgccgccccgggcccacccgcaagcattaccagccctatgccccaccacgcg
acttcgcagcctatcgctccTTCAGCAGAAGCGCCGACGCCCCTGCCTACCAGCAGG
GCCAGAATCAGCTGTACAACGAGCTGAACCTGGGCAGAAGGGAAGAGTACGACGTCC
TGGATAAGCGGAGAGGCCGGGACCCTGAGATGGGCGGCAAGCCTCGGCGGAAGAACC
CCCAGGAAGGCCTGTATAACGAACTGCAGAAAGACAAGATGGCCGAGGCCTACAGCG
AGATCGGCATGAAGGGCGAGCGGAGGCGGGGCAAGGGCCACGACGGCCTGTATCAGG
GCCTGTCCACCGCCACCAAGGATACCTACGACGCCCTGCACATGCAGGCCCTGCCCCCAAGG
CD20CAR full sequence (protein):
(SEQ ID NO: 15548)
METDTLLLWVLLLWVPGSTGDIVLTQSPAILSASPGEKVTMTCRASSSVNYMDWYQKK
PGSSPKPWIYATSNLASGVPARFSGSGSGTSYSLTISRVEAEDAATYYCQQWSFNPPTFG
GGTKLEIKGSTSGGGSGGGSGGGGSSEVQLQQSGAELVKPGASVKMSCKASGYTFTSY
NMHWVKQTPGQGLEWIGAIYPGNGDTSYNQKFKGKATLTADKSSSTAYMQLSSLTSED
SADYYCARSNYYGSSYWFFDVWGAGTTVTVSSESKYGPPCPPCPMFWVLVVVGGVLA
CYSLLVTVAFIIFWVRSKRSRLLHSDYMNMTPRRPGPTRKHYQPYAPPRDFAAYRSFSRS
ADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR

### Example 15: Evaluation of Gene Editing of Primary Human T Cells with GFP and a BCMA CAR by a Retrotransposon-Based Gene Modifying System

The Vingi-1_Acar gene modifying system comprising a BCMA-CAR template RNA and a GFP template RNA was introduced into primary human CD3+ T cells. The BCMA CAR template RNA for use with the gene modifying polypeptide comprised the following sequences from 5′ to 3′: Vingi 5′UTR, tkPA, BCMA CAR, EF1a short promoter, Vingi 3′ UTR. The GFP template RNA for use with the gene modifying polypeptide comprised the following sequences from 5′ to 3′: Vingi 5′UTR, tkPA, GFP, EF1a short promoter, Vingi 3′ UTR. The sequence of the EF1a short promoter is included below.

EF1a short promoter:
(SEQ ID NO: 15545)
gggcagagcgcacatcgcccacagtccccgagaagttggggggaggggtc
ggcaattgatccggtgcctagagaaggtggcgcggggtaaactgggaaag
tgatgtcgtgtactggctccgcctttttcccgaggggggggagaaccgta
tataagtgcagtagtcgccgtgaacgttctttttcgcaacgggtttgccg
ccagaacacag

Briefly, human CD3+ T cells stored in liquid nitrogen from 3 donors were thawed in TexMACS culture media (Mitenyi) supplemented with 5% Human AB serum, IL2 (20 IU/mL; Miltenyi), IL7 (10 ng/mL; Miltenyi), and IL15 (5 ng/mL; Miltenyi). Cells were activated immediately post thaw by supplementing the culture media with TransACT (according to manufacturer's protocol) for two days.

Activated T cells were electroporated with mRNA encoding Vingi-1_Acar gene modifying polypeptide (as described in Example 5), BCMA-CAR template RNA, and GFP template RNA at 48 hours post activation. As a control, T cells were electroporated with only Vingi-1_Acar driver under the same conditions. For the electroporation, 5 million T cells per condition were electroporated with 500 μg/mL of RNA (mass ratio of 1 template:1 template:1 driver) in OptiMEM to maintain a cell concentration of 4×10⁸ cells/mL. Post electroporation, conditions were quenched in expansion media (TexMACS, 20 IU/mL IL2, 10 ng/mL of IL7, 5 ng/mL of IL15, 5% Human AB serum) and expanded until day 10 post initial thaw. Test and control conditions were analyzed by flow cytometry for CAR and GFP expression on days 5, 7, and 10 post thaw.

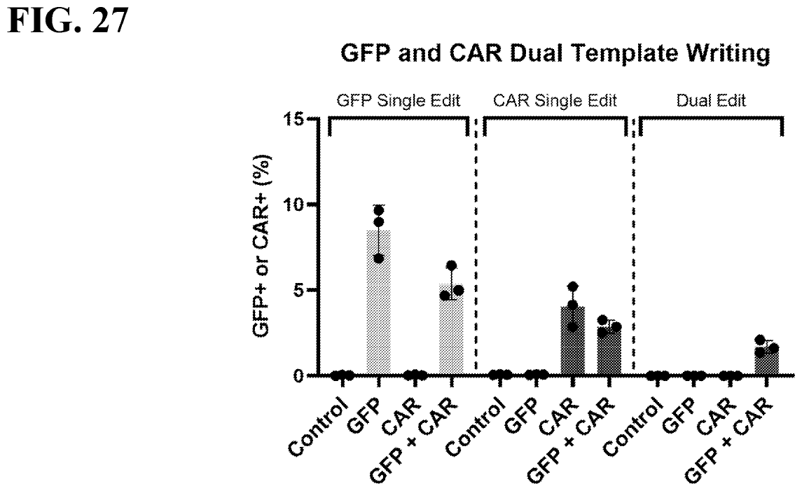

As shown in FIG. 27, the Vingi-1_Acar gene modifying system introduced both the BCMA CAR and GFP in primary human T cells obtained from 3 different donors. On average, around 2% of activated T cells co-electroporated with the mRNA encoding the Vingi-1_Acar gene modifying polypeptide, the BCMA_CAR template RNA, and the GFP template RNA expressed both the CAR and GFP at 5 days post electroporation. In comparison, cells treated with mRNA encoding Vingi-1_Acar gene modifying polypeptide alone, mRNA encoding the Vingi-1_Acar gene modifying polypeptide plus the BCMA_CAR template RNA or mRNA encoding the Vingi-1_Acar gene modifying polypeptide plus the GFP template RNA did not show any CAR-GFP double positive expression. These results demonstrate that Vingi-1_Acar gene modifying systems can be utilized to insert and express multiple transgenes into primary human T cells.

What is claimed is:

1. A system for modifying DNA comprising:
(a) a gene modifying polypeptide or a nucleic acid (e.g., DNA or mRNA) encoding the gene modifying polypeptide, and
(b) a template RNA (or DNA encoding the template RNA) comprising (i) a sequence that binds the polypeptide and (ii) a heterologous object sequence encoding a chimeric antigen receptor (CAR), wherein the CAR comprises an antigen-binding domain, a transmembrane domain, a first intracellular signaling domain, and a second intracellular signaling domain.

2. A system for modifying DNA comprising:
(a) a gene modifying polypeptide or a nucleic acid (e.g., DNA or mRNA) encoding the gene modifying polypeptide, and
(b) a template RNA (or DNA encoding the template RNA) comprising (i) a sequence that binds the polypeptide and (ii) a heterologous object sequence encoding a chimeric antigen receptor (CAR), wherein one or more of:
(i) the CAR comprises an antigen binding domain that binds one or more antigens of a blood cancer (e.g., a leukemia or lymphoma), wherein optionally the antigen is a B cell antigen;
(ii) the CAR comprises an antigen binding domain that binds one or more antigens of a solid tumor;
(iii) the CAR comprises an antigen binding domain of any one of Tables C1-C5 or C9;
(iv) the CAR comprises a linker domain of Table L1 (e.g., a linker of SEQ ID NO 15520);
(v) the CAR comprises a transmembrane domain of Table C6 or C6A;
(vi) the CAR comprises a hinge domain (e.g., a hinge domain of Table C8);
(vii) the CAR comprises an intracellular signaling domain of Table C7 or C7A;
(viii) the CAR comprises a costimulatory domain of Table C7 or C7A;
(ix) the CAR comprises an antigen binding domain which comprises an scFv, a Fab, a diabody, a D domain binder, a centryin, or one or more single domain antibodies (e.g., VHH domains); or
(x) the CAR comprises an amino acid sequence of Table C9 or an amino acid sequence according to any one of SEQ ID NOs: 1100, 15490, 15492, 15498, 15500, 15502, 15503, 15505, 15507, 15509 and 15510, 15555, 15557 and 15558, 15559, 15560, 15561, 15515, 15526, 15531, 15536, 15541, or 15548;
(xi) wherein the CAR comprises a first intracellular signaling domain and a second intracellular signaling domain.

3. A population of cells comprising immune effector cells and/or regulatory immune cells, the population comprising:
a plurality of copies of a gene encoding a CAR (“a CAR gene”), wherein less than or equal to 70%, 65%, 60%, or 55% of copies of the CAR gene in the population are situated within a gene endogenous to a cell of the population.

4. A population of cells comprising immune effector cells and/or regulatory immune cells, the population comprising:
a plurality of copies of a gene encoding a CAR (“a CAR gene”), wherein
at least 20%, 25%, 30%, 35%, or 40% of copies of the CAR gene in the population are situated within an intergenic region endogenous to a cell of the population.

5. A method of modifying the genome of a mammalian cell, comprising contacting the cell with a system of claim 1 or 2, thereby modifying the genome of the mammalian cell.

6. A reaction mixture comprising:
a system of claim 1 or 2, and
a mammalian cell.

7. A cell or population of cells produced by the method of claim 5.

8. A method of treating a cancer in a subject in need thereof, the method comprising administering to the subject a cell or population of cells of any of claims 3, 4, or 7.

9. A cell or population of cells of any of claims 3, 4, or 7, or the system of claim 1 or 2, for use in treating a cancer.

10. Use of a cell or population of cells of any of claims 3, 4, or 7, or the system of claim 1 or 2, in the manufacture of a medicament for treating a cancer.

11. A method of treating a cancer in a subject in need thereof, the method comprising contacting an immune effector cell and/or a regulatory immune cell of the subject with a system of claim 1 or 2.

12. A gene modifying polypeptide comprising an amino acid sequence of SEQ ID NO: 420, or a sequence having at least 70%, 75%, 80%, 85%, 90%, 95%, 96%, 97%, 98%, or 99% identity thereto, wherein amino acid position 191 is other than D, e.g., is A, or a fragment thereof having reverse transcriptase activity.

13. A gene modifying polypeptide comprising an amino acid sequence of SEQ ID NO: 421, or a sequence having at least 70%, 75%, 80%, 85%, 90%, 95%, 96%, 97%, 98%, or 99% identity thereto, wherein amino acid position 250 is other than D, e.g., is A, or a fragment thereof having reverse transcriptase activity.

14. A nucleic acid encoding a gene modifying polypeptide of claim 12 or 13.

15. A method of modifying the genome of a mammalian induced pluripotent stem cell (iPSC), the method comprising contacting the cell with:
(a) a gene modifying polypeptide, or a nucleic acid (e.g., DNA or mRNA) encoding the gene modifying polypeptide, and
(b) a template RNA (or DNA encoding the template RNA) comprising (i) a sequence that binds the polypeptide and (ii) a heterologous object sequence.

16. The method of any of claims 5, 8, 11, or 15, wherein the DNA damage response (DDR) pathway in the cell (e.g., an iPSC) is not activated, or is activated less than in an otherwise similar cell treated with Cas9, e.g., in an assay according to Example 2.

17. The method of any of claims 5, 8, 11, 15, or 16, wherein the interferon response is not activated, or is activated less than in an otherwise similar cell treated with a gene modifying system comprising elements from a LINE-1 retrotransposase, e.g., in an assay according to Example 3.

18. A method of modifying the genome of a mammalian respiratory epithelial cell (e.g., a bronchial epithelial cell, e.g., a human bronchial epithelial (hBE) cell), the method comprising contacting the cell with:
(a) a gene modifying polypeptide, or a nucleic acid (e.g., DNA or mRNA) encoding the gene modifying polypeptide, and
(b) a template RNA (or DNA encoding the template RNA) comprising (i) a sequence that binds the polypeptide and (ii) a heterologous object sequence.

19. A lipid nanoparticle (LNP) composition comprising the system of claim 1 or 2.

20. A system for modifying DNA comprising:
(a) a first gene modifying system comprising: (i) a retrotransposon gene modifying polypeptide or a nucleic acid (e.g., DNA or mRNA) encoding the retrotransposon gene modifying polypeptide, and (ii) a first template RNA (or DNA encoding the template RNA) comprising (1) a sequence that binds the polypeptide and (2) a first heterologous object sequence; and
(b) a second system comprising: (i) a heterologous gene modifying polypeptide or a nucleic acid (e.g., DNA or mRNA) encoding the heterologous gene modifying polypeptide, and (ii) a second template RNA (or DNA encoding the template RNA) comprising (1) a gRNA spacer, (2) a gRNA scaffold, (3) a heterologous object sequence, and (4) a primer binding site (PBS) sequence.

21. The system of claim 20, wherein the second system further comprises a third template RNA (or DNA encoding the template RNA) comprising (1) a gRNA spacer, (2) a gRNA scaffold, (3) a heterologous object sequence, and (4) a primer binding site (PBS) sequence.

22. The system of claim 20 or 21, wherein the retrotransposon gene modifying polypeptide comprises an amino acid sequence of Table R1 or a sequence having no more than 10, 20, 30, 40, 50, 60, 70, 80, 90, or 100 amino acid differences thereto, or a nucleic acid (e.g., DNA or mRNA) encoding the retrotransposon gene modifying polypeptide.

23. The system of any one of claims 20-22, wherein the retrotransposon gene modifying polypeptide comprises an amino acid sequence listed in any of Examples 6-10 or a sequence having no more than 10, 20, 30, 40, 50, 60, 70, 80, 90, or 100 amino acid differences thereto, or a nucleic acid (e.g., DNA or mRNA) encoding the retrotransposon gene modifying polypeptide.

24. The system of any of claims 20-23, wherein the sequence that binds the polypeptide comprises a 5′UTRᵣₑₜᵣₒ or a 3′ UTRᵣₑₜᵣₒ.

25. The system of claim 24, wherein the first template RNA comprises both of a 5′UTRᵣₑₜᵣₒ and a 3′ UTRᵣₑₜᵣₒ.

26. The system of claim 24, wherein the 5′UTRᵣₑₜᵣₒ and the 3′ UTRᵣₑₜᵣₒ comprise 5′ or 3′ sequences of Table R1 or any of Examples 6-10.

27. The system of any of claims 20-26, wherein the first heterologous object sequence encodes a chimeric antigen receptor (CAR), wherein the CAR comprises an antigen-binding domain, a transmembrane domain, a first intracellular signaling domain, and a second intracellular signaling domain.

28. The gene modifying system of any of claims 20-27, wherein the heterologous gene modifying polypeptide comprises:
a reverse transcriptase (RT) domain (e.g., an RT domain from a retrovirus, or a polypeptide domain having at least 75%, 80%, 85%, 90%, 95%, 96%, 97%, 98%, or 99% amino acids sequence identity thereto); and
a Cas domain that binds to the target DNA molecule and is heterologous to the RT domain (e.g., a Cas9 domain); and optionally, a linker disposed between the RT domain and the Cas domain.

29. The system of claim 28, wherein the RT domain comprises an amino acid sequence of Table 6, or a sequence having at least 70%, 80%, 85%, 90%, 95%, 97%, 98%, or 99% identity thereto.

30. The gene modifying system of claim 28 or 29, wherein the Cas domain comprises a Cas domain of Table 7 or Table 8A, or a sequence having at least 70%, 80%, 85%, 90%, 95%, 97%, 98%, or 99% identity thereto.

31. A method of modifying the genome of a mammalian cell, comprising contacting a population of mammalian cells with a system of any one of claims 20-30, thereby modifying the genome of a cell of the population.

32. The method of claim 31, wherein the first gene modifying system produces a first sequence alteration (e.g., an insertion) and the second system produces a second sequence alteration in the genome of the mammalian cell.

33. The method of claim 32, wherein at least 5%, 10%, or 20% of cells in the population comprise the first sequence alteration.

34. The method of any 32 or 33, wherein at least 10%, 20%, 30%, 40%, 50%, or 60% of cells in the population comprise the second sequence alteration.

35. The method of any one of claims 32-34, wherein at least 20%, 40%, 60%, 80% of cells that comprise the first sequence alteration also comprise the second sequence alteration.

36. The method of any one of claims 32-35, wherein the modifying does not result in a translocation event.

37. A template RNA comprising from 5′ to 3′:
(i) a gRNA spacer that is complementary to a first portion of the human TRAC gene;
(ii) a gRNA scaffold that binds a heterologous gene modifying polypeptide (e.g., binds the Cas domain of the heterologous gene modifying polypeptide),
(iii) a heterologous object sequence comprising a mutation region to introduce a mutation into (e.g., to correct a mutation in) a second portion of the human TRAC gene (wherein optionally the heterologous object sequence comprises, from 5′ to 3′, a post-edit homology region, a mutation region, and a pre-edit homology region), and
(iv) a primer binding site (PBS) sequence comprising at least 5, 6, 7, or 8 bases with 100% identity to a third portion of the human TRAC gene,
wherein the template RNA comprises a nucleotide sequence according to SEQ ID NO: 15,000 or 15,001.
